(19) United States
(12) Patent Application Publication
(10) Pub. No.: US 2026/0356971 A1
(43) Pub. Date: Nov. 12, 2026

(54) NOVEL ANTI-MESOTHELIN CHIMERIC ANTIGEN RECEPTORS AND MODIFIED IMMUNE CELLS

(71) Applicant: Carisma Therapeutics Inc., Philadelphia, PA (US)

(72) Inventors: Nicholas Roy Anderson, Merion Station, PA (US); Thomas Claude Henri Condamine, Wynnewood, PA (US); Michael Klichinsky, New Hope, PA (US); Daniel Blumenthal, Merion Station, PA (US); Nicholas G. Minutolo, Philadelphia, PA (US); David Christopher Sloas, Philadelphia, PA (US); Yumi Ohtani, Wallingford, PA (US)

(21) Appl. No.: 19/117,932

(22) PCT Filed: Oct. 2, 2023
(86) PCT No.: PCT/US2023/075737

§ 371 Date: Apr. 2, 2025

Related U.S. Application Data
(60) Provisional application No. 63/412,622, filed on Oct. 3, 2022.
(60) Provisional application No. 63/459,903, filed on Apr. 17, 2023.

(51) Int. Cl. C07K 16/30; A61K 40/17; A61K 40/31; A61K 40/42; C07K 14/705; C12N 5/0786; C12N 13/00; C12N 15/113; C12N 15/86

(52) U.S. Cl. CPC C07K 16/30; A61K 40/17; A61K 40/31; A61K 40/4255; C07K 14/7051; C07K 14/70517; C07K 14/70521; C07K 14/70535; C07K 14/70578; C12N 5/0645; C12N 13/00; C12N 15/113; C12N 15/86; C07K 2317/622; C07K 2319/03; C12N 2310/531; C12N 2510/00; C12N 2710/10043

(57) ABSTRACT

The present disclosure pertains to modified immune cells comprising anti-mesothelin chimeric antigen receptors and methods of using and making immune cells comprising anti-mesothelin chimeric antigen receptors.

### CROSS REFERENCE TO RELATED APPLICATIONS

This application claims priority to and the benefit of U.S. Provisional Patent Application Nos. 63/412,622, filed Oct. 3, 2023 and 63/459,903, filed Apr. 17, 2023, the contents of which are incorporated herein by reference in their entirety.

### BACKGROUND

Mesothelin is a tumor antigen that is highly expressed in many human cancers, including malignant mesothelioma and pancreatic, ovarian, and lung adenocarcinomas. It is a target of interest for cancer immunotherapy because its normal expression in humans is limited to mesothelial cells. Therefore, a need exists for the development of new therapeutic modalities optimized to target mesothelin.

### SUMMARY OF THE INVENTION

The present disclosure encompasses, among other things, compositions comprising modified immune cells (e.g., stem cells, macrophages, monocytes, and/or dendritic cells) comprising anti-mesothelin chimeric antigen receptors (CARs) and methods of producing and using the same.

In one aspect, the present disclosure provides modified immune cells comprising a chimeric antigen receptor (CAR), wherein the CAR comprises: (a) an extracellular domain; (b) a transmembrane domain; and (c) one or more intracellular domains; wherein the extracellular domain is or comprises an anti-mesothelin antigen binding domain comprising an amino acid sequence that is at least 80% identical to a sequence selected from Table 3; and wherein the modified immune cell is or comprises a macrophage, monocyte, dendritic cell, or stem cell.

In some embodiments, an extracellular domain is or comprises an scFv, VHH antibody, centyrin, darpin, or nanobody. In some embodiments, a transmembrane domain is or comprises a CD8, CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain. In some embodiments, one or more intracellular domains comprise: a CD3ζ, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, IL1R, IL2R, IL3R, IL4R, IL5R, IL6R, IL7R, IL8R, IL9R, IL10R, IL11R, IL12R, IL13R, IL14R, IL15R, IL17R, IFNaR, IFNgR, TNFR, CSF1R, CSF2R, Dap10, CD36, Dectin-1, ICOSL, or Syk intracellular domain, a portion of any of the foregoing, or combinations thereof. In some embodiments, one or more intracellular domains comprise a CD3ζ intracellular domain or an FcRγ intracellular domain.

In some embodiments, a CAR further comprises an extracellular leader domain. In some embodiments, an extracellular leader domain comprises a CD8a extracellular leader domain. In some embodiments, a CAR further comprises an extracellular hinge domain. In some embodiments, an extracellular hinge domain comprises: a CD8 extracellular hinge domain, a CD8a extracellular hinge domain, a CD28 extracellular hinge domain, a DNGR-1 extracellular hinge domain, a Dectin-1 extracellular hinge domain, or an IgG4 extracellular hinge domain.

In some embodiments, a CAR comprises, from N-terminus to C-terminus: a CD8a leader domain, an anti-mesothelin antigen binding domain, a CD8 extracellular hinge domain, a CD8 transmembrane domain, and a CD3ζ intracellular domain; a CD8a leader domain, an anti-mesothelin antigen binding domain, a CD28 extracellular hinge domain, a CD28 transmembrane domain, and a CD3ζ intracellular domain; a CD8a leader domain, an anti-mesothelin antigen binding domain, a CD28 extracellular hinge domain, a CD28 transmembrane domain, and an FcRγ intracellular domain; a CD8a leader domain, an anti-mesothelin antigen binding domain, a CD8 extracellular hinge domain, a CD8 transmembrane domain, a CD3ζ intracellular domain, a P2A cleavage peptide, and a CD40 ligand (CD40L); a CD8a leader domain, an anti-mesothelin antigen binding domain, a CD28 extracellular hinge domain, a CD28 transmembrane domain, a CD3ζ intracellular domain, a P2A cleavage peptide, and a CD40 ligand (CD40L); a CD8a leader domain, an anti-mesothelin antigen binding domain, a CD28 extracellular hinge domain, a CD28 transmembrane domain, a MyD88 intracellular domain, and a CD3ζ intracellular domain; a CD8a leader domain, an anti-mesothelin antigen binding domain, a CD28 extracellular hinge domain, a CD28 transmembrane domain, a MyD88 intracellular domain, a CD40 intracellular domain, and a CD3ζ intracellular domain; a CD8a leader domain, an anti-mesothelin antigen binding domain, a CD28 extracellular hinge domain, a CD28 transmembrane domain, a truncated MyD88 intracellular domain, a CD40 intracellular domain, and a CD3ζ intracellular domain; a CD8a leader domain, an anti-mesothelin antigen binding domain, a CD28 extracellular hinge domain, a CD28 transmembrane domain, an FcRγ intracellular domain, a P2A cleavage peptide, and a CD40 ligand (CD40L); a CD8a leader domain, an anti-mesothelin antigen binding domain, a CD28 extracellular hinge domain, a CD28 transmembrane domain, a MyD88 intracellular domain, and an FcRγ intracellular domain; a CD8a leader domain, an anti-mesothelin antigen binding domain, a CD28 extracellular hinge domain, a CD28 transmembrane domain, a MyD88 intracellular domain, a CD40 intracellular domain, and an FcRγ intracellular domain; or a CD8a leader domain, an anti-mesothelin antigen binding domain, a CD28 extracellular hinge domain, a CD28 transmembrane domain, a truncated MyD88 intracellular domain, a CD40 intracellular domain, and an FcRγ intracellular domain.

In some embodiments, a CAR has or comprises: (a) an amino acid sequence selected from Table 2; (b) an amino acid sequence that differs from a sequence selected from Table 2 by no more than five substitutions, additions, or deletions; or (c) an amino acid sequence that is at least 80% identical to a sequence selected from Table 2.

In another aspect, the present disclosure provides a pharmaceutical composition comprises a modified immune cell as described herein. In some embodiments, a pharmaceutical composition comprises a pharmaceutically acceptable carrier.

In another aspect, the present disclosure provides a nucleic acid construct comprising one or more nucleic acid sequences encoding: (a) an extracellular binding domain; (b) a transmembrane domain; and (c) one or more intracellular domains; wherein the extracellular domain is or comprises an anti-mesothelin antigen binding domain comprising an amino acid sequence that is at least 80% identical to a sequence selected from Table 5; and wherein the nucleic acid construct encodes a chimeric antigen receptor (CAR) comprising (a) through (c). In some embodiments, a nucleic acid construct further comprises one or more nucleic acid sequences encoding: (d) one or more extracellular leader domains, (e) one or more extracellular hinge domains, (f) one or more cleavage peptides, or combinations thereof. In some embodiments, a cleavage peptide is or comprises a P2A, F2A, E2A or T2A peptide.

In some embodiments, a nucleic acid construct encodes, from N-terminus to C-terminus: a CD8a leader domain, an anti-mesothelin antigen binding domain, a CD8 extracellular hinge domain, a CD8 transmembrane domain, and a CD3ζ intracellular domain; a CD8a leader domain, an anti-mesothelin antigen binding domain, a CD28 extracellular hinge domain, a CD28 transmembrane domain, and a CD3ζ intracellular domain; a CD8a leader domain, an anti-mesothelin antigen binding domain, a CD28 extracellular hinge domain, a CD28 transmembrane domain, and an FcRγ intracellular domain; a CD8a leader domain, an anti-mesothelin antigen binding domain, a CD8 extracellular hinge domain, a CD8 transmembrane domain, a CD3ζ intracellular domain, a P2A cleavage peptide, and a CD40 ligand (CD40L); a CD8a leader domain, an anti-mesothelin antigen binding domain, a CD28 extracellular hinge domain, a CD28 transmembrane domain, a CD3ζ intracellular domain, a P2A cleavage peptide, and a CD40 ligand (CD40L); a CD8a leader domain, an anti-mesothelin antigen binding domain, a CD28 extracellular hinge domain, a CD28 transmembrane domain, a MyD88 intracellular domain, and a CD3ζ intracellular domain; a CD8a leader domain, an anti-mesothelin antigen binding domain, a CD28 extracellular hinge domain, a CD28 transmembrane domain, a MyD88 intracellular domain, a CD40 intracellular domain, and a CD3ζ intracellular domain; a CD8a leader domain, an anti-mesothelin antigen binding domain, a CD28 extracellular hinge domain, a CD28 transmembrane domain, a truncated MyD88 intracellular domain, a CD40 intracellular domain, and a CD3ζ intracellular domain; a CD8a leader domain, an anti-mesothelin antigen binding domain, a CD28 extracellular hinge domain, a CD28 transmembrane domain, an FcRγ intracellular domain, a P2A cleavage peptide, and a CD40 ligand (CD40L); a CD8a leader domain, an anti-mesothelin antigen binding domain, a CD28 extracellular hinge domain, a CD28 transmembrane domain, a MyD88 intracellular domain, and an FcRγ intracellular domain; a CD8a leader domain, an anti-mesothelin antigen binding domain, a CD28 extracellular hinge domain, a CD28 transmembrane domain, a MyD88 intracellular domain, a CD40 intracellular domain, and an FcRγ intracellular domain; or a CD8a leader domain, an anti-mesothelin antigen binding domain, a CD28 extracellular hinge domain, a CD28 transmembrane domain, a truncated MyD88 intracellular domain, a CD40 intracellular domain, and an FcRγ intracellular domain.

In some embodiments, a nucleic acid construct has or comprises: (a) a nucleotide sequence selected from Table 4; (b) a nucleotide sequence that differs from a sequence selected from Table 4 by no more than five substitutions, additions, or deletions; or (c) a nucleotide acid sequence that is at least 80% identical to a sequence selected from Table 4.

In some embodiments, a nucleic acid construct of the present disclosure further comprises one or more introns, wherein the one or more introns comprise one or more inhibitory nucleic acids, and wherein the one or more inhibitory nucleic acids encode one or more inhibitory RNAs. In some embodiments, one or more inhibitory RNAs are or comprise one or more shRNA. In some embodiments, one or more shRNA comprise a guide strand. In some embodiments, a guide strand comprises a nucleic acid sequence that is reverse complementary to a target gene transcript comprising a target nucleic acid sequence. In some embodiments, a target gene transcript encodes human ATG7, C/EBP-alpha, C/EBP-beta, CD32b, CD36, CLEC1A, FATS, GOLM1, HAVCR2, ITGAD, KLF4, KLF6, LILRB1, LILRB2, LILRB4, MAF, MafB, PD1, PD-LI, PIK3CG, PIK3CG, PPARα, PPARγ, PTGS2, SIGLEC10, SIRPα, SLAMF3, SLAMF4, SLC15A3, STAT3, STAT6, TNFRSF1B, TOX, TREM2, YTHDF2, or ZFP36. In some embodiments, a target gene transcript encodes a human anti-phagocytic receptor selected from the group consisting of: SIRPα, LILRB1, SIGLEC10, PD1, SLAMF3, SLAMF4, CLEC1A, and CD32b. In some embodiments, a target gene transcript encodes human SIRPα.

In another aspect, the present disclosure provides a pharmaceutical composition comprises a nucleic acid construct as described herein. In some embodiments, a pharmaceutical composition comprises a pharmaceutically acceptable carrier.

In another aspect, the present disclosure provides methods of treating a disease or disorder in a subject, the methods comprising: administering to the subject a therapeutically effective amount of a pharmaceutical composition as described herein, wherein at least one sign or symptom of the disease or disorder is improved in the subject after administration. In some embodiments, a step of administering is or comprises transarterial, subcutaneous, intravenous, intradermal, intratumoral, intranodal, intramedullary, intramuscular, or intraperitoneal delivery.

In another aspect, the present disclosure provides methods of modifying an immune cell, the methods comprising: delivering to the immune cell a nucleic acid construct as described herein, thereby producing a modified immune cell, wherein the modified immune cell is or comprises a macrophage, monocyte, dendritic cell, or stem cell.

In some embodiments, a nucleic acid construct comprises DNA or messenger RNA (mRNA). In some embodiments, a nucleic acid construct comprises a modification selected from: a modified nucleotide, an alteration to the 5′ untranslated region (UTR), an alteration to the 3′ UTR, a cap structure, a poly(A) tail, or combinations thereof. In some embodiments, a cap structure comprises AGCap1, m6AGCap1, or Anti-Reverse Cap Analog (ARCA). In some embodiments, a modified nucleotide comprises pseudouridine (PsU), 5-methoxyuridine (5moU), 5-methylcytidine/pseudouridine (5meC PsU), N1-methyl-pseudouridine (NimPsU), or combinations thereof.

In some embodiments, a nucleic acid construct is a purified nucleic acid construct. In some embodiments, a purified nucleic acid construct is produced by a method comprising silica membrane purification, high performance liquid chromatography (HPLC), Dynabeads, LiCl precipitation, phenol-chloroform extraction, resin based purification, polyA isolation, RNeasy, or combinations thereof. In some embodiments, a nucleic acid construct is codon-optimized. In some embodiments, a nucleic acid construct is codon-optimized for expression in a stem cell, monocyte, macrophage, or dendritic cell.

In some embodiments, delivering comprises electroporation or transfection with the nucleic acid construct. In some embodiments, a nucleic acid construct is encapsulated within a delivery vehicle. In some embodiments, a delivery vehicle is or comprises a liposome, a lipid nanoparticle, a polymer, an adeno-associated viral (AAV) vector, an adenoviral vector, a retroviral vector or combinations thereof. In some embodiments, a liposome or lipid nanoparticle comprises one or more cationic lipids, one or more non-cationic lipids, one or more cholesterol-based lipids, one or more PEG-modified lipids, or combinations thereof. In some embodiments, a retroviral vector comprises a lentiviral vector or a gammaretroviral vector. In some embodiments, a lentiviral vector is packaged with a Vpx protein. In some embodiments, an adenoviral vector comprises an Ad2 vector or an Ad5 vector. In some embodiments, an Ad5 vector comprises an Ad5f35 adenoviral vector.

In some embodiments, a method of the present disclosure further comprises delivering to the immune cell an additional payload. In some embodiments, an additional payload is or comprises a pathogen recognition receptor agonist, polyinosinic:polycytidylic acid (poly I:C), a TLR7/8 agonist, a CpG oligodeoxynucleotide, a NOD-like receptor (NLR) agonist, a RIG-1-like receptor (RLR) agonist, a C-type lectins receptor (CLR) agonist, a cytosolic DNA sensing, the cyclic GMP-AMP synthase-stimulator of interferon genes (cGAS-STING) agonist, an interferon-inducible protein 16 (IFI16) agonist, a DEAD-box helicase 41 (DDX41) agonist, an LRR binding FLII interacting protein 1 (LRRFIP1) agonist, an absent in melanoma 2 (AIM2) agonist, an aryl hydrocarbon receptor (AhR) ligand, or combinations thereof. In some embodiments, a nucleic acid construct and an additional payload are encapsulated within a delivery vehicle.

### BRIEF DESCRIPTION OF THE DRAWINGS

The drawings are for illustration purposes only, not for limitation.

FIG. 1A, FIG. 1B, and FIG. 1C show graphs of exemplary macrophage viability (FIG. 1A) and CAR expression (FIG. 1B and FIG. 1C) after electroporation with mRNA encoding 20 different anti-mesothelin binders (M1-M20) that were used in a CD8 framework chimeric antigen receptor (CAR).

FIG. 2 shows a graph of exemplary target cell killing mediated by macrophages after electroporation with mRNA encoding 4 different anti-mesothelin binders (M11, M14, M15, and M17) that were used in a CD8 framework CAR.

FIG. 3 shows a graph of exemplary TNFα cytokine release mediated by macrophages after electroporation with mRNA encoding 4 different anti-mesothelin binders (M11, M14, M15, and M17) that were used in a CD8 framework CAR.

FIG. 4 shows a graph of exemplary anti-mesothelin mediated phagocytosis by macrophages after electroporation with mRNA encoding 4 different anti-mesothelin binders (M11, M14, M15, and M17) that were used in a CD8 framework CAR.

FIG. 5 shows a graph of exemplary macrophage viability after transduction with varying exemplary MOIs of Ad5f35 vector comprising CTX_269 (an anti-mesothelin CAR).

FIG. 6 shows graphs of exemplary anti-mesothelin CAR expression after transduction of macrophages with varying exemplary MOIs of Ad5f35 vector comprising CTX_269 (an anti-mesothelin CAR).

FIG. 7A, FIG. 7B, and FIG. 7C shows graphs of exemplary expression of exemplary M1-associated markers (CD80, CD86, and HLA-DR) after transduction of macrophages with varying exemplary MOIs of Ad5f35 vector comprising CTX_269 (an anti-mesothelin CAR).

FIG. 8 shows graphs of exemplary macrophage expression of exemplary M2-associated markers (CD163 and CD206) after transduction of macrophages with varying exemplary MOIs of Ad5f35 vector comprising CTX_269 (an anti-mesothelin CAR).

FIG. 9 shows graphs of exemplary M2-associated marker (CD163) expression after transduction of macrophages with Ad5f35 vector comprising CTX_269 (an anti-mesothelin CAR).

FIG. 10 shows graphs of exemplary cytokine M1-associated marker (CD86) expression after transduction of macrophages with Ad5f35 vector comprising CTX_269 (an anti-mesothelin CAR).

FIG. 11 shows graphs of exemplary M2-associated markers (CD163 and CD206) expression after transduction of macrophages with Ad5f35 vector comprising CTX_269 (an anti-mesothelin CAR) and exposure of the macrophages to mesothelin.

FIG. 12 shows a graph of exemplary anti-mesothelin mediated phagocytosis of A549 lung adenocarcinoma cells by macrophages after transduction of macrophages with Ad5f35 vector comprising CTX_269 (an anti-mesothelin CAR).

FIG. 13 shows a graph of exemplary anti-mesothelin mediated phagocytosis of MES-OV ovarian cystadenocarcinoma cells by macrophages after transduction of macrophages with Ad5f35 vector comprising CTX_269 (an anti-mesothelin CAR).

FIG. 14 shows graphs of exemplary anti-mesothelin mediated killing of mesothelin-expressing A549 lung adenocarcinoma cells by macrophages after transduction of the macrophages with Ad5f35 vector comprising CTX_269 (an anti-mesothelin CAR).

FIG. 15 shows graphs of exemplary anti-mesothelin mediated killing of mesothelin-expressing ovarian cystadenocarcinoma cells by macrophages after transduction of the macrophages with Ad5f35 vector comprising CTX_269 (an anti-mesothelin CAR).

FIG. 16 shows graphs of exemplary cytokine (TNFα and IL-1β) release after transduction of macrophages with Ad5f35 vector comprising CTX_269 (an anti-mesothelin CAR) and exposure of the macrophages to mesothelin.

FIG. 17 shows graphs of exemplary cytokine (TNFα) release after transduction of macrophages with Ad5f35 vector comprising CTX_269 (an anti-mesothelin CAR) and exposure of the macrophages to target cells (A549 lung adenocarcinoma cells or MES-OV ovarian cystadenocarcinoma cells) expressing mesothelin.

FIG. 18 shows an exemplary experimental timeline for treatment of an in vivo mouse tumor model with macrophages transduced with Ad5f35 vector comprising CTX_269 (an anti-mesothelin CAR).

FIG. 19 shows exemplary graphs of tumor burden in mice treated with macrophages transduced with Ad5f35 vector comprising CTX_269 (an anti-mesothelin CAR).

FIG. 20 shows graphs of exemplary anti-mesothelin CAR expression after transduction of macrophages with lentivirus vector comprising a CD28-based anti-mesothelin CAR or with lentivirus vector comprising a CD8-based anti-mesothelin CAR.

FIG. 21 shows a graph of exemplary target cell killing mediated by macrophages after transduction of macrophages with lentivirus vector comprising a CD28-based anti-mesothelin CAR or with lentivirus vector comprising a CD8-based anti-mesothelin CAR.

FIG. 22 shows graphs of exemplary cytokine (TNFα) release after transduction of macrophages with lentivirus vector comprising a CD28-based anti-mesothelin CAR or with lentivirus vector comprising a CD8-based anti-mesothelin CAR and exposure of the transduced macrophages to mesothelin.

FIG. 23 shows graphs of exemplary M2-associated markers (CD163 and CD206) expression after transduction of macrophages with Ad5f35 vector comprising either CTX_269 (an anti-mesothelin CAR comprising a CD8-framework) or CTX_293 (an anti-mesothelin CAR comprising a CD28-framework) and exposure of the transduced macrophages to IL-10.

FIG. 24 shows graphs of exemplary M1-associated markers (CD80 and CD86) expression after transduction of macrophages with Ad5f35 vector comprising either CTX_269 (an anti-mesothelin CAR comprising a CD8-framework) or CTX_293 (an anti-mesothelin CAR comprising a CD28-framework) and exposure of the transduced macrophages to IL-10.

FIG. 25 shows a graph of exemplary anti-mesothelin mediated killing of mesothelin-expressing A549 lung adenocarcinoma cells by macrophages after transduction of the macrophages with Ad5f35 vector comprising either CTX_269 (an anti-mesothelin CAR comprising a CD8-framework) or CTX_293 (an anti-mesothelin CAR comprising a CD28-framework).

FIG. 26A, FIG. 26B, FIG. 26C, FIG. 26D, FIG. 26E, and FIG. 26F show graphs of exemplary phenotypic markers for monocytes transduced with either CTX_269 (a CD8-based anti-mesothelin CAR) or CTX_001 (an anti-HER2 CAR), monocytes transduced with CTX_269 or CTX_001 and then differentiated into macrophages, and macrophages transduced with CTX_269 or CTX_001 after differentiation from monocytes.

FIG. 27 shows graphs of exemplary anti-mesothelin mediated killing of mesothelin-expressing ovarian cystadenocarcinoma cells (left graph) or mesothelin-expressing A549 lung adenocarcinoma cells (right graph) by monocytes transduced with either CTX_269 (a CD8-based anti-mesothelin CAR) or CTX_001 (an anti-HER2 CAR), monocytes transduced with CTX_269 or CTX_001 and then differentiated into macrophages, and macrophages transduced with CTX_269 or CTX_001 after differentiation from monocytes.

FIG. 28 shows schematics of exemplary anti-mesothelin CAR constructs comprising an M15 scFv.

FIG. 29 shows schematics of exemplary anti-mesothelin CAR constructs comprising an M17 scFv.

FIG. 30 shows graphs of exemplary anti-mesothelin CAR expression on days 2 and 14 after transduction of macrophages with Ad5f35 vector comprising CTX_269 (an anti-mesothelin CAR).

FIG. 31 shows graphs of exemplary anti-mesothelin mediated phagocytosis of K562 cells by macrophages after transduction of macrophages with Ad5f35 vector comprising CTX_269 (an anti-mesothelin CAR).

FIG. 32 shows graphs of exemplary cytokine (TNFα) release after transduction of macrophages with Ad5f35 vector comprising CTX_269 (an anti-mesothelin CAR) and exposure of the macrophages to target cells (K562 cells) expressing mesothelin.

FIG. 33 shows representative tissue sections of murine lungs immunohistochemistry (IHC) stained for human mesothelin in mice treated with macrophages transduced with Ad5f35 vector comprising CTX_269 (an anti-mesothelin CAR). Scale bar 1 mm.

FIG. 34 shows exemplary tumor nodule quantifications from murine lungs treated with macrophages transduced with Ad5f35 vector comprising CTX_269 (an anti-mesothelin CAR).

FIG. 35A and FIG. 35B show graphs of exemplary anti-mesothelin CAR expression and cell viability after transduction of monocytes with Ad5f35 virus comprising CTX_964 (an anti-mesothelin CAR) or CTX_1461 (an anti-mesothelin CAR+intronic shRNA against SIRPα).

FIG. 36A and FIG. 36B show graphs of exemplary SIRPa expression after transduction of monocytes with Ad5f35 virus comprising CTX_964 (an anti-mesothelin CAR) or CTX_1461 (an anti-mesothelin CAR+intronic shRNA against SIRPα).

FIG. 37 shows graphs of exemplary cytokine (TNFα) release after transduction of monocytes with Ad5f35 virus comprising CTX_964 (an anti-mesothelin CAR) or CTX_1461 (an anti-mesothelin CAR+intronic shRNA against SIRPα) and exposure of the cells to either recombinant human mesothelin or recombinant mesothelin+recombinant human CD47.

FIG. 38A and FIG. 38B show graphs of exemplary anti-mesothelin mediated killing of mesothelin-expressing target cells by monocytes and macropahges after transduction of the cells with Ad5f35 vector comprising either CTX_964 (an anti-mesothelin CAR) or CTX_1461 (an anti-mesothelin CAR+intronic shRNA against SIRPα).

FIG. 39 shows a graph of exemplary suppression of tumor growth by CAR-monocyte-derived CAR macrophages after transduction of the cells with Ad5f35 vector comprising either CTX_964 (an anti-mesothelin CAR) or CTX_1461 (an anti-mesothelin CAR+intronic shRNA against SIRPα).

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Activation: As used herein, the term “activation” refers to the state of a cell, for example a monocyte, macrophage, or dendritic cell that has been sufficiently stimulated to induce detectable cellular proliferation or has been stimulated to exert its effector function. Activation can also be associated with induced cytokine production, phagocytosis, cell signaling, target cell killing, and/or antigen processing and presentation.

Activated monocytes/macrophages dendritic cells: As used herein, the term “activated monocytes/macrophages/dendritic cells” refers to, among other things, monocyte/macrophage/dendritic cells that are undergoing cell division or exerting effector function. The term “activated monocytes/macrophages/dendritic cells” refers to, among others thing, cells that are performing an effector function or exerting any activity not seen in the resting state, including phagocytosis, cytokine secretion, proliferation, gene expression changes, metabolic changes, and other functions.

Agent: As used herein, the term “agent” (or “biological agent” or “therapeutic agent”), refers to a molecule that may be expressed, released, secreted or delivered to a target by a modified cell described herein. An agent includes, but is not limited to, a nucleic acid, an antibiotic, an anti-inflammatory agent, an antibody or fragments thereof, an antibody agent or fragments thereof, a growth factor, a cytokine, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a chimeric switch receptor or a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is not and/or does not comprise a polypeptide whose amino acid sequence includes structural elements recognized by those skilled in the art as an immunoglobulin variable domain. In some embodiments, an antibody agent may be or comprise a molecule or composition which does not include immunoglobulin structural elements (e.g., a receptor or other naturally occurring molecule which includes at least one antigen binding domain).

Antibody fragment: As used herein, the term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments and human and humanized versions thereof.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Anti-tumor effect: As used herein, the term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of a tumor in the first place.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Xenogenic: As used herein, the term “xenogeneic” refers to any material (e.g., a population of cells) derived from an animal of a different species.

Cancer: As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand. As used herein, the term “co-stimulatory ligand” refers to a molecule on an antigen presenting cell (e.g., an APC, dendritic cell, B cell, and the like) that specifically binds a cognate co-stimulatory molecule on a monocyte/macrophage/dendritic cell, thereby providing a signal which mediates a monocyte/macrophage/dendritic cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a monocyte/macrophage/dendritic cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of macrophages.

Effective amount: As used herein, “effective amount” and “therapeutically effective amount” are interchangeable, and refer to an amount of a compound, formulation, material, or composition, as described herein effective to achieve a particular biological result or provides a manufacturing, therapeutic or prophylactic benefit. Such results may include, but are not limited to, anti-tumor activity as determined by any means suitable in the art.

Effector function: As used herein, “effector function” or “effector activity” refers to a specific activity carried out by an immune cell in response to stimulation of the immune cell. For example, an effector function of macrophages to engulf and digest cellular debris, foreign substances, microbes, cancer cells and other unhealthy cells by phagocytosis.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, monocytes, macrophages, and/or dendritic cells. In one embodiment, monocytes, macrophages, or dendritic cells that are expanded ex vivo increase in number relative to the number originally present in a culture. In another embodiment, monocytes, macrophages, or dendritic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Immune cell: As used herein, the term “immune cell,” refers to a cell that is involved in an immune response, e.g., promotion of an immune response. Examples of immune cells include, but are not limited to, macrophages, monocytes, dendritic cells, neutrophils, eosinophils, mast cells, platelets, large granular lymphocytes, Langerhans' cells, natural killer (NK) cells, T-lymphocytes, or B-lymphocytes. A source of immune cells (e.g., macrophages, monocytes, or dendritic cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Lentivirus: As used herein, the term “lentivirus” refers to a genus of the Retroviridae family. Lentiviruses are unique among the retroviruses in being able to infect non-dividing cells; they can deliver a significant amount of genetic information into the DNA of a host cell, so they are one of the most efficient methods of a gene delivery vector. HIV, SIV, and FIV are all examples of lentiviruses. Vectors derived from lentiviruses offer the means to achieve significant levels of gene transfer in vivo.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Overexpressed tumor antigen: As used herein, the term “overexpressed” tumor antigen or “overexpression” of a tumor antigen refers to an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

Specifically binds: As used herein, the term “specifically binds,” with respect to an antigen binding domain, such as an antibody agent, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

Stimulation: As used herein, the term “stimulation,” refers to a primary response induced by binding of a stimulatory molecule (e.g., an FcR complex, a TLR complex, or a TCR/CD3 complex), for example, with its cognate ligand thereby mediating a signal transduction event, such as, but not limited to, signal transduction via Fc receptor machinery, via a chimeric switch receptor, or via a synthetic CAR. Stimulation can mediate altered expression of certain molecules, such as downregulation of TGF-beta, and/or reorganization of cytoskeletal structures, and the like. As used herein, the term “stimulatory molecule,” refers to a molecule of a monocyte, macrophage, or dendritic cell that specifically binds with a cognate stimulatory ligand present on an antigen presenting cell. In some embodiments, a stimulatory molecule comprises an FcR extracellular domain comprising a CD64 (FcγRI), CD32a (FcγRIIa), CD32b (FcγRIIb), CD32c, CD16a (FcγRIIIa), CD16b (FcγRIIIb), FcεRI, FcεRII, FcαRI (CD89) or CD40 domain. In some embodiments, a stimulatory molecule comprises a TLR extracellular domain comprising a TLR1, TLR2, TLR3, TLR4, TLR5, TLR6, TLR7, TLR8, or TLR9 domain. As used herein, the term “stimulatory ligand,” refers to a ligand that when present on an antigen presenting cell (e.g., an aAPC, a macrophage, a dendritic cell, a B-cell, and the like) or tumor cell can specifically bind with a cognate binding partner (referred to herein as a “stimulatory molecule”) on a monocyte, macrophage, or dendritic cell thereby mediating a response by the immune cell, including, but not limited to, activation, initiation of an immune response, proliferation, and the like. Stimulatory ligands are well-known in the art and encompass, inter alia, Toll-like receptor (TLR) ligand, an anti-toll-like receptor antibody, an agonist, and an antibody for a monocyte/macrophage receptor. In addition, cytokines, such as interferon-gamma, are potent stimulants of macrophages.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially purified: As used herein, the term “substantially purified”, for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, an antibody (or fragment thereof), a chimeric switch receptor, or a CAR.

Target site: As used herein, the term “target site” or “target sequence” refers to a genomic nucleic acid sequence that defines a portion of a nucleic acid to which a binding molecule may specifically bind under conditions sufficient for binding to occur.

T cell receptor: As used herein, the term “T cell receptor” or “TCR” refers to a complex of membrane proteins that participate in the activation of T cells in response to the presentation of antigen. A TCR is responsible for recognizing antigens bound to major histocompatibility complex molecules. A TCR comprises a heterodimer of an alpha (a) and beta (β) chain, although in some cells the TCR comprises gamma and delta (γ/δ) chains. TCRs may exist in alpha/beta and gamma/delta forms, which are structurally similar but have distinct anatomical locations and functions. Each chain comprises two extracellular domains, a variable and constant domain. In some embodiments, a TCR may be modified on any cell comprising a TCR, including, for example, a helper T cell, a cytotoxic T cell, a memory T cell, regulatory T cell, natural killer T cell, and gamma delta T cell.

Therapeutic: As used herein, the term “therapeutic” refers to a treatment and/or prophylaxis. A therapeutic effect is obtained by suppression, remission, or eradication of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to an immune cell (e.g., a monocyte, macrophage, or dendritic cell) or contacting an immune cell with a modulator of a pathway activated by in vitro transcribed mRNA.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

The present disclosure encompasses, among other things, compositions comprising modified immune cells (e.g., stem cells, macrophages, monocytes, and/or dendritic cells) comprising novel chimeric antigen receptors (CARs) described herein and methods of using and producing the same. The present disclosure also encompasses, among other things, compositions comprising modified immune cells (e.g., stem cells, macrophages, monocytes, and/or dendritic cells) comprising novel nucleic acid constructs encoding CARs described herein and methods of using and producing the same. In some embodiments, CARs of the present disclosure comprise an anti-mesothelin antigen binding domain as described herein. In some embodiments, CARs of the present disclosure comprise one or both of (i) a CD8 or CD28 extracellular hinge domain, and (ii) a CD8 or CD28 transmembrane domain.

In some embodiments, modified immune cells described herein comprising or expressing CARs described herein exhibit increased tumor killing, e.g., relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, modified immune cells described herein comprising or expressing CARs described herein do not exhibit killing of tumor cells that do not express a target antigen (e.g., mesothelin) e.g., relative to modified immune cells of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, tumor killing comprises or is one or more of phagocytosis, lysis, apoptosis, or production of tumor killing cytokines (e.g., TNFα). In some embodiments, modified immune cells described herein comprising or expressing CARs described herein exhibit increased tumor killing for a specific length of time. In some embodiments, modified immune cells described herein comprising or expressing CARs described herein exhibit increased tumor killing for at least one week. In some embodiments, modified immune cells described herein comprising or expressing CARs described herein exhibit increased tumor killing for at least two weeks.

In some embodiments, modified immune cells described herein comprising or expressing CARs described herein exhibit increased viability, e.g., relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, modified immune cells described herein comprising or expressing CARs described herein exhibit increased expression of a CAR, e.g., relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, modified immune cells described herein comprising or expressing CARs described herein exhibit increased expression of M1 markers (e.g., one or both of CD80 or CD86), e.g., relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, modified immune cells described herein comprising or expressing CARs described herein exhibit decreased expression of M2 markers (e.g., one or both of CD163 or CD206), e.g., relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a CAR described herein comprises: (a) an extracellular domain comprising an anti-mesothelin binding domain as described herein; (b) a transmembrane domain (e.g., a CD28 transmembrane domain or CD8 transmembrane domain); and (c) one or more intracellular domains. In some embodiments, one or more intracellular domains comprise a CD3zeta (CD3ζ) intracellular domain. In some embodiments, one or more intracellular domains comprise an FcRγ intracellular domain. In some embodiments, a CAR further comprises one or more extracellular hinge domains. In some embodiments, one or more extracellular hinge domains comprise a CD28 extracellular hinge domain or a CD8a extracellular hinge domain.

In some embodiments, a CAR described herein comprises, from N-terminus to C-terminus: a CD8a leader domain, an anti-mesothelin antigen binding domain, a CD8 extracellular hinge domain, a CD8 transmembrane domain, and a CD3ζ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, and a CD3ζ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, and an FcRγ intracellular domain. In some embodiments, a CAR described herein comprises, from N-terminus to C-terminus: a CD8a leader domain, an anti-mesothelin antigen binding domain, a CD8 extracellular hinge domain, a CD8 transmembrane domain, a CD3ζ intracellular domain, a P2A cleavage peptide, and a CD40 ligand (CD40L). In some embodiments, a CAR described herein comprises, from N-terminus to C-terminus: a CD8a leader domain, an anti-mesothelin antigen binding domain, a CD28 extracellular hinge domain, a CD28 transmembrane domain, a CD3ζ intracellular domain, a P2A cleavage peptide, and a CD40 ligand (CD40L). In some embodiments, a CAR described herein comprises, from N-terminus to C-terminus: a CD8a leader domain, an anti-mesothelin antigen binding domain, a CD28 extracellular hinge domain, a CD28 transmembrane domain, a MyD88 intracellular domain, and a CD3ζ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, a MyD88 intracellular domain, a CD40 intracellular domain, and a CD3ζ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, a truncated MyD88 intracellular domain, a CD40 intracellular domain, and a CD3ζ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, an FcRγ intracellular domain, a P2A cleavage peptide, and a CD40 ligand (CD40L). In some embodiments, a CAR described herein comprises, from N-terminus to C-terminus: a CD8a leader domain, an anti-mesothelin antigen binding domain, a CD28 extracellular hinge domain, a CD28 transmembrane domain, a MyD88 intracellular domain, and an FcRγ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, a MyD88 intracellular domain, a CD40 intracellular domain, and an FcRγ intracellular domain. In some embodiments, a CAR described herein comprises, from N-terminus to C-terminus: a CD8a leader domain, an anti-mesothelin antigen binding domain, a CD28 extracellular hinge domain, a CD28 transmembrane domain, a truncated MyD88 intracellular domain, a CD40 intracellular domain, and an FcRγ intracellular domain.

### Immune Cells

The present disclosure provides, among other things, modified immune cells (e.g., stem cells, macrophages, monocytes, or dendritic cells) comprising at least one chimeric antigen receptor (CAR) described herein. In some embodiments, a population of immune cells described herein comprises stem cells, monocytes, macrophages, dendritic cells, and/or precursors thereof. In some embodiments, a population of immune cells comprises a substantially purified population of stem cells, monocytes, macrophages, or dendritic cells, or a cell line.

In some embodiments, an immune cell is activated, e.g., an immune cell exhibits increased cytokine production, chemokine production, phagocytosis, cell signaling, target cell killing, and/or antigen presentation, e.g., relative to an inactive cell. In some embodiments, an activated immune cell exhibits changes in gene expression, e.g., an induction of pro-inflammatory gene expression, e.g., relative to an inactive cell. In some embodiments, an activated immune cell exhibits changes in gene expression, e.g., an induction of anti-inflammatory gene expression, e.g., relative to an inactive cell. In certain embodiments, activated immune cells are undergoing cell division. In some embodiments, targeted effector activity of an immune cell is enhanced by inhibition of CD47 and/or SIRPa activity. CD47 and/or SIRPa activity may be inhibited by treating an immune cell with an anti-CD47 or anti-SIRPa antibody or by any method known to those skilled in the art.

In some embodiments, immune cells (e.g., stem cells, macrophages, monocytes, or dendritic cells) are obtained (e.g., isolated) from a subject. Cells can be obtained from a number of sources including peripheral blood mononuclear cells, bone marrow, lymph node tissue, spleen tissue, umbilical cord, tumors, and/or induced pluripotent stem cells, such as embryonic stem cells (ESCs). In certain embodiments, cells can be obtained from a unit of blood collected from a subject using any number of separation techniques known to a skilled artisan, such as Ficoll separation. In some embodiments, cells from circulating blood of a subject are obtained by apheresis or leukapheresis. Cells collected by apheresis may be washed to remove a plasma fraction and resuspended in a variety of buffers (e.g., phosphate buffered saline (PBS)) or culture media). In some embodiments, enrichment of immune cells (e.g. monocytes) comprises plastic adherence. In some embodiments, following enrichment, differentiation of immune cells (e.g. monocytes) comprises stimulation with GM-CSF. In some embodiments, a composition comprising blood cells (e.g., monocytes, lymphocytes, platelets, plasma, and/or red blood cells), such as a leukapheresis composition (e.g., a leukopak) is used for enrichment. In some embodiments, a leukapheresis composition (e.g., a leukopak) comprises a sample from a healthy human donor. In certain embodiments, apheresis of immune cells (e.g. monocytes) is followed by mobilization with GM-CSF. In certain embodiments, selection of immune cells (e.g., monocytes) comprises CD14 positive selection using microbeads (e.g., MACS® MicroBeads on a CliniMACS Prodigy device). In some embodiments, an immune cell precursor (e.g., precursors to macrophages, monocytes, or dendritic cells including, but not limited to induced pluripotent stem cells, or iPSCs) is used in compositions and methods described herein. Immune cell precursors may be differentiated in vivo or ex vivo into immune cells. Non-limiting examples of precursor immune cells include hematopoietic stem cells, common myeloid progenitors, myeloblasts, monoblasts, promonocytes, or intermediates thereof. For example, induced pluripotent stem cells may be used to generate monocytes, macrophages, and/or dendritic cells. Induced pluripotent stem cells (iPSCs) may be derived from normal human tissue, such as peripheral blood, fibroblasts, skin, keratinocytes, or renal epithelial cells. Autologous, allogeneic, or universal donor iPSCs could be differentiated toward a myeloid lineage (e.g., a monocyte, macrophage, dendritic cell, or precursor thereof).

Immune cells (e.g., stem cells, macrophages, monocytes, or dendritic cells) described herein can be isolated from peripheral blood, for example, by lysing red blood cells and depleting lymphocytes and red blood cells, such as by centrifugation through a PERCOLL™ gradient. Alternatively, immune cells can be isolated from umbilical cord tissue. A specific subpopulation of immune cells can be further isolated by positive or negative selection techniques. In some embodiments, immune cells can be depleted of cells expressing certain antigens, including, but not limited to, CD34, CD3, CD4, CD8, CD56, CD66b, CD19, or CD20. In some embodiments, enrichment of an immune cell population, for example, by negative selection can be accomplished using a combination of antibodies directed to surface markers unique to the negatively selected cells. By way of non-limiting example, cell selection can also comprise negative magnetic immunoadherence or flow cytometry that uses a cocktail of monoclonal antibodies directed to cell surface markers present on negatively selected cells.

During isolation of a desired population of immune cells (e.g., stem cells, macrophages, monocytes, or dendritic cells) described herein by positive or negative selection, immune cell concentration and surface (e.g., particles, such as beads) can be varied. It may be desirable to significantly decrease volume in which beads and cells are mixed together to ensure maximum contact area of cells and beads.

In some embodiments, prior to administration, modified immune cells (e.g., stem cells, macrophages, monocytes, or dendritic cells) described herein (e.g., comprising at least one CAR described herein) are treated with a pro-inflammatory agent. In some embodiments, treatment with a pro-inflammatory agent increases anti-tumor activity of modified immune cells described herein. In some embodiments, treatment with at least one pro-inflammatory agent promotes M1 phenotype (e.g., a switch from M2 to M1 phenotype) in modified immune cells described herein. In some embodiments, at least one pro-inflammatory agent comprises or is a CD40 agonist (e.g., CD40L). In some embodiments, at least one pro-inflammatory agent comprises or is a 41BB-ligand agonist (e.g., 4-1BB). In some embodiments, at least one pro-inflammatory agent comprises or is a CD40 agonist (e.g., CD40L) and a 41BB-ligand agonist (e.g., 4-1BB).

In some embodiments, modified immune cells (e.g., stem cells, macrophages, monocytes, or dendritic cells) described herein (e.g., comprising at least one CAR described herein) have been treated with one or more pro-inflammatory agents. In some embodiments, a modified immune cell described herein exhibits increased anti-tumor activity relative to an unmodified cell of the same type. In some embodiments, one or more pro-inflammatory agents comprises or is a CD40 agonist (e.g., CD40L). In some embodiments, one or more pro-inflammatory agents comprises or is a 41BB-ligand agonist (e.g., 4-1BB). In some embodiments, one or more pro-inflammatory agents comprises or is a CD40 agonist (e.g., CD40L) and a 41BB-ligand agonist (e.g., 4-1BB). The disclosure provides methods of treating a disease or disorder in a subject, comprising: delivering to the subject a therapeutically effective amount of a pharmaceutical composition comprising a modified macrophage, monocyte, or dendritic cell described herein.

The disclosure also provides methods of modifying immune cells (e.g., stem cells, macrophages, monocytes, or dendritic cells) described herein comprising a CAR described herein, wherein the method comprises treating an immune cell described herein with one or more pro-inflammatory agents, thereby producing a modified immune cell described herein that exhibits increased anti-tumor activity relative to an immune cell of the same type comprising the CAR or a similar CAR that has not been treated with one or more pro-inflammatory agents. In some embodiments, one or more pro-inflammatory agents comprises or is a CD40 agonist (e.g., CD40L). In some embodiments, one or more pro-inflammatory agents comprises or is a 41BB-ligand agonist (e.g., 4-1BB). In some embodiments, one or more pro-inflammatory agents comprises or is a CD40 agonist (e.g., CD40L) and a 41BB-ligand agonist (e.g., 4-1BB). The disclosure provides methods of treating a disease or disorder in a subject, comprising: delivering to the subject a therapeutically effective amount of a pharmaceutical composition comprising immune cells described herein modified by methods described herein.

In some embodiments, modified immune cells (e.g., stem cells, macrophages, monocytes, or dendritic cells) described herein (e.g., comprising a CAR described herein) are administered to a subject in combination with a pro-inflammatory agent. In some embodiments, modified immune cells (e.g., stem cells, macrophages, monocytes, or dendritic cells) described herein (e.g., comprising a CAR described herein) are administered to a subject substantially simultaneously, before, or after a pro-inflammatory agent. In some embodiments, a pro-inflammatory agent is administered as a nucleic acid (e.g., in a construct packaged with a CAR and a cleavage peptide such as a P2A, F2A, E2A and/or T2A peptide). In some embodiments, administration with a pro-inflammatory agent increases anti-tumor activity of modified immune cells described herein. In some embodiments, administration with a pro-inflammatory agent promotes M1 phenotype (e.g., a switch from M2 to M1 phenotype) in immune cells described herein. In some embodiments, a pro-inflammatory agent comprises or is a CD40 agonist (e.g., CD40L). In some embodiments, a pro-inflammatory agent comprises or is a 41BB-ligand agonists (e.g., 4-1BB).

### Macrophages

Macrophages are immune cells specialized for detection, phagocytosis, and destruction of target cells, such as pathogens or tumor cells. Macrophages are potent effectors of the innate immune system and are capable of at least three distinct anti-tumor functions: 1) phagocytosis of dead and dying cells, microorganisms, cancer cells, cellular debris, or other foreign substances; 2) cytotoxicity against tumor cells; and 3) presentation of tumor antigens to orchestrate an adaptive anti-tumor immune response.

Macrophages are abundant in the tumor microenvironment of numerous cancers and can adopt a number of phenotypes that are collectively referred to as tumor-associated macrophages (TAMs). The immunosuppressive nature of the tumor microenvironment typically results in more M2-like TAMs, which further contribute to the general suppression of anti-tumor immune responses. Recent studies, however, have identified that TAMs are able to be “reprogrammed” via pro-inflammatory signals, and that the switch from a M2 phenotype to a more M1 phenotype is associated with productive anti-tumor immune responses. Inducing endogenous TAMs to switch to M1-type cells and engineering macrophages that cannot be subverted into M2 would greatly enhance anti-tumor immunotherapy and represent a significant advance in the field.

In some embodiments, a macrophage comprises or is an undifferentiated or M0 macrophage. In certain embodiments, a macrophage comprises or expresses one, two, three, four, five, or six of CD14, CD16, CD64, CD68, CD71, or CCR5. Exposure to various stimuli can induce M0 macrophages to polarize into several distinct populations, which may be identified by macrophage phenotype markers, cytokine production, and/or chemokine secretion.

In some embodiments, a macrophage comprises or is a polarized macrophage. Under classical conditions of activation, M0 macrophages can be exposed to pro-inflammatory signals, such as LPS, IFNγ, and GM-CSF, and polarize into pro-inflammatory (i.e., M1) macrophages. Generally, pro-inflammatory (M1) macrophages are associated with pro-inflammatory immune responses, such as Th1 and Th17 T cell responses. Exposure to other stimuli can polarize macrophages into a diverse group of “alternatively activated” or anti-inflammatory (i.e., M2) macrophages.

In some embodiments, a macrophage comprises or is a pro-inflammatory (M1) macrophage. In some embodiments, a macrophage expresses one or more markers of pro-inflammatory (M1) macrophages (e.g., 1, 2, 3, 4, 5, 6, 7, 8, 9, 10, 11, 12, 13, 14, 15, 16, 17, or 18 of CD86, CD80, MHC II, IL-1R, TLR2, TLR4, iNOS, SOCS3, CD83, PD-L1, CD69, MHC I, CD64, CD32, CD16, IL1R, a IFIT family member, or an ISG family member).

In some embodiments, a macrophage comprising or expressing at least one CAR described herein secretes relatively high levels of one or more inflammatory cytokines (e.g., 1, 2, 3, 4, 5, 6, 7, 8, 9, 10, 11, or 12 of IL-1, TNF, IL-12, IL-18, IL-23, IFNα, IFNβ, IFNγ, IL-2, IL-6, IL-8, or IL33) or chemokines (e.g., one or both of CC or CXC chemokines) (e.g., 1, 2, 3, 4, 5, 6, 7, 8, 9, 10, 11, 12, 13, 14, 15, or 16 of the CXC chemokines; e.g., 1, 2, 3, 4, 5, 6, 7, 8, 9, 10, 11, 12, 13, 14, 15, 16, 17, 18, 19, 20, 21, 22, 23, 24, 25, 26, 27, or 28 of the CC chemokines; eg., one of the CX3C chemokines, e.g., one or both of the C chemokines), e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein stimulates an immune response and/or inflammation, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a macrophage comprises or is an anti-inflammatory (M2) macrophage (e.g., an M2a, M2b, M2c, and M2d macrophage). An M2a macrophage can be induced by IL-4, IL-13, and/or fungal infection. An M2b macrophage can be induced by IL-1R ligands, an immune complex, and/or LPS. An M2c macrophage can be induced by IL-10 and/or TGFβ. An M2d macrophage can be induced by IL-6 and/or adenosine. In some embodiments, a macrophage comprising or expressing at least one CAR described herein decreases an immune response in a subject, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage expresses one or more markers of anti-inflammatory (M2) macrophages (e.g., one, two, or three of CD206, CD163, or CD209). In some embodiments, a macrophage comprising or expressing at least one CAR described herein exhibits increased secretion of one or more anti-inflammatory cytokines (e.g., one or both of IL-10 or TGFβ), e.g., relative to a macrophage comprising a similar CAR (e.g., a CAR described herein).

In some embodiments, a macrophage comprises at least one upregulated pro-inflammatory (M1) marker and/or at least one downregulated anti-inflammatory (M2) marker as compared to a control macrophage that does not comprise at least one CAR as provided herein and/or the same macrophage before delivery of at least one CAR described herein. In some embodiments, at least one pro-inflammatory (M1) marker (e.g., HLA DR, CD86, CD80, PD-L1, CD83, CD69, MHC I, CD64, CD32, CD16, IL1R, an IFIT family member, and/or an ISG family member) is upregulated in a macrophage. In some embodiments, at least one anti-inflammatory (M2) marker (e.g., CD206, CD163, and/or CD209) is downregulated in a macrophage.

In some embodiments, a macrophage comprising or expressing at least one CAR described herein exhibits increased phagocytosis, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits increased cytotoxicity against a tumor cell, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits increased tumor antigen presentation (e.g., post-phagocytosis presentation) and/or increased antigen processing, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR exhibits increased tumor killing (e.g., by phagocytosis, lysis, apoptosis, or production of tumor killing cytokines (e.g., TNFα)), e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a macrophage comprising or expressing at least one CAR described herein exhibits one or both of increased expression of one or more genes typically associated with increased effector function (e.g., phagocytosis, targeted cellular cytotoxicity, antigen presentation, or cytokine secretion) (e.g., CD80, CD86, MHC-I, MHC-II, CD40, 41BBL, TNF, IFN-α, IFN—P, IFN-γ, IL2, IL12, IL6, IL8, IL1b, and/or CXCL12) or decreased expression of one or more genes typically associated with decreased effector function (e.g., phagocytosis, targeted cellular cytotoxicity, antigen presentation, or cytokine secretion) (e.g., CD163, CD206, TGFβ, IL-10, and/or IL4), e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits increased production of ROS, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits metabolic reprogramming (e.g., of an interferon signaling pathway, TH1 pathway, PTEN signaling, PI3K signaling, MTOR signaling, TLR signaling, CD40 signaling, 41BB signaling, 41BBL signaling, macrophage maturation signaling, dendritic cell maturation signaling, CD3-zeta signaling, FcR γ signaling, CD64 signaling, CD32a signaling, CD32c signaling, CD16a signaling, TLR1 signaling, TLR2 signaling, TLR3 signaling, TLR4 signaling, TLR5 signaling, TLR6 signaling, TLR7 signaling, TLR8 signaling, TLR9 signaling, ALK signaling, AXL signaling, DDR2 signaling, EGFR signaling, EphA1 signaling, INSR signaling, cMET signaling, MUSK signaling, PDGFR signaling, PTK7 signaling, RET signaling, ROR1 signaling, ROS1 signaling, RYK signaling, TIE2 signaling, TRK signaling, VEGFR signaling, CD40 signaling, CD19 signaling, CD20 signaling, 41BB signaling, CD28 signaling, OX40 signaling, GITR signaling, TREM-1 signaling, TREM-2 signaling, DAP12 signaling, MR signaling, ICOS signaling, MyD88 signaling, V/I/LxYxxL/V signaling, SIRPa signaling, CD45 signaling, Siglec-10 signaling, PD1 signaling, SHP-1 signaling, SHP-2 signaling, KIR-2DL signaling, KIR-3DL signaling, NKG2A signaling, CD170 signaling, CD33 signaling, BTLA signaling, CD32b signaling, SIRPβ signaling, CD22 signaling, PIR-B signaling, and/or LILRB1 signaling), e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits induction of cell survival mechanisms, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits induction of cell death mechanisms, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits one, two, three, four, or five of increased resistance to phagocytic checkpoints, increased expression of chemokine receptors to aid in trafficking, increased expression of chemokines to recruit other immune cells, increased expression of ECM degrading enzymes (e.g., MMPs to degrade tumor ECM and/or exhibit anti fibrotic activity), and/or increased proliferation, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a macrophage comprising or expressing at least one CAR described herein exhibits one, two, three, or four of improved duration of CAR expression, improved stability of the CAR on the cell surface, increased level of CAR expression, and/or decreased background activity of the CAR, e.g., relative to a macrophage comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

### Monocytes

Monocytes are multipotent cells that circulate in the blood, bone marrow, and spleen, and generally do not proliferate when in a steady state. Monocytes can vary in size significantly in the range of about 10-30 μm in diameter. A ratio of nucleus to cytoplasm for a monocyte can range from about 2:1 to about 1:1. Typically, monocytes comprise chemokine receptors and pathogen recognition receptors that mediate migration from blood to tissues, such as during an infection. Monocytes can produce inflammatory cytokines, take up cells and/or toxic molecules, and differentiate into dendritic cells or macrophages.

In some embodiments, a monocyte comprises or expresses one or more phenotypic markers. Exemplarily phenotypic markers for human monocyte cells include, but are not limited to, CD9, CD11b, CD11c, CDw12, CD13, CD15, CDw17, CD31, CD32, CD33, CD35, CD36, CD38, CD43, CD49b, CD49e, CD49f, CD63, CD64, CD65s, CD68, CD84, CD85, CD86, CD87, CD89, CD91, CDw92, CD93, CD98, CD101, CD102, CD111, CD112, CD115, CD116, CD119, CDwl2lb, CDw123, CD127, CDw128, CDw131, CD147, CD155, CD156a, CD157, CD162 CD163, CD164, CD168, CD171, CD172a, CD180, CD206, CD131a1, CD213 2, CDw210, CD226, CD281, CD282, CD284, and CD286. Exemplarily phenotypic markers for mouse monocyte cells include, but are not limited to, CD11a, CD11b, CD16, CD18, CD29, CD31, CD32, CD44, CD45, CD49d, CD115, CD116, Cdw131, CD281, CD282, CD284, CD286, F4/80, and CD49b. In certain embodiments, monocytes comprise one, two, or three of CD11b, CD14, or CD16. In certain embodiments, monocytes comprise CD14+CD16− monocytes, CD14+CD16+ monocytes, or CD14− CD16+ monocytes.

In some embodiments, a monocyte differentiates into a macrophage. In some embodiments, a monocyte differentiates into a dendritic cell (DC). Monocytes can be differentiated into macrophages or DCs by any technique known in the art. For example, differentiation of monocytes into macrophages can be induced by macrophage colony stimulating factor (M-CSF). Differentiation of monocytes into DCs can be induced by granulocyte-macrophage colony stimulating factor (GM-CSF) in combination with IL-4.

In some embodiments, a monocyte comprising or expressing at least one CAR described herein exhibits increased secretion of one or more cytokines (e.g., one, two, three, four, five, six, or seven of TNF, IL-12, IFN, GM-CSF, G-CSF, M-CSF, or IL-1), e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits increased phagocytosis, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits enhanced survival, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits enhanced differentiation into macrophages (e.g., M1 or M2 macrophages), e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits enhanced differentiation into DCs (e.g., resident or migrating DCs and/or in lymphoid and non-lymphoid tissue), e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits increased cytotoxicity against a tumor cell, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits increased tumor antigen presentation (e.g., post-phagocytosis presentation) and/or increased antigen processing, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits increased tumor killing (e.g., by phagocytosis, lysis, apoptosis, or production of tumor killing cytokines (e.g., TNFα), e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a monocyte comprising or expressing at least one CAR described herein exhibits one or both of increased expression of one or more genes typically associated with increased effector function (e.g., phagocytosis, targeted cellular cytotoxicity, antigen presentation, or cytokine secretion) or decreased expression of one or more genes typically associated with decreased effector function (e.g., phagocytosis, targeted cellular cytotoxicity, antigen presentation, or cytokine secretion), e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits increased production of ROS, e.g., relative to a monocyte without a CAR described herein. In some embodiments, a monocyte comprising or expressing at least one CAR described herein exhibits metabolic reprogramming, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits induction of cell survival mechanisms, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits induction of cell death mechanisms, e.g., relative to a monocyte comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a monocyte comprising or expressing at least one CAR described herein exhibits one, two, three, four, or five of: increased resistance to phagocytic checkpoints, increased expression of chemokine receptors to aid in trafficking, increased expression of chemokines to recruit other immune cells, increased expression of ECM degrading enzymes (e.g., MMPs to degrade tumor ECM and/or exhibit anti fibrotic activity), and/or increased proliferation, e.g., relative to a monocyte without a CAR described herein. In some embodiments, a monocyte comprising or expressing at least one CAR described herein exhibits one, two, three, or four of: improved duration of CAR expression, improved stability of the CAR on the cell surface, increased level of CAR expression, and/or decreased background activity of the CAR, e.g., relative to a monocyte without a CAR described herein.

### Dendritic Cells

Dendritic cells (DCs) are bone marrow-derived, specialized antigen presenting cells that are involved in initiating immune responses and maintaining tolerance of the immune system to self-antigens. Dendritic cells may be found in both lymphoid and non-lymphoid organs and are generally thought to arise from lymphoid or myeloid lineages.

In some embodiments, a DC comprises or expresses one or more phenotypic markers. Exemplarily phenotypic markers for DCs include, but are not limited to, CD11c, CD83, CD1a, CD1c, CD141, CD207, CLEC9a, CD123, CD85, CD180, CD187, CD205, CD281, CD282, CD284, CD286 and partially CD206, CD207, CD208 and CD209.

Immature DCs can be characterized by a high capacity for antigen capture, but relatively low T cell stimulatory capability. Inflammatory mediators promote DC maturation. Once DCs reach the mature stage, there is a dramatic change in properties relative to immature DCs, such as a decrease in antigen capture ability and/or an increased ability to stimulate T cells. In some embodiments, a DC comprises or is an immature DC. In other embodiments, a DC comprises or is a mature DC.

Without wishing to be bound by theory, it is believed that modification of a DC cell to comprise or express at least one CAR described herein can allow mature DCs to simultaneously exhibit increased antigen capture ability and T cell stimulation, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein mediates tumor antigen presentation, e.g., increased tumor antigen presentation relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein mediates tumor T cell stimulation, e.g., increased T cell stimulation relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a DC comprising or expressing at least one CAR described herein exhibits increased secretion of one or more cytokines (e.g., one, two, three, four, five, six, or seven of TNF, IL-12, IFN, GM-CSF, G-CSF, M-CSF, or IL-1), e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein exhibits increased phagocytosis, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein exhibits increased tumor antigen presentation (e.g., post-phagocytosis presentation), increased antigen processing, increased antigen cross presentation, increased T cell priming, and/or stimulation of T cells, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a DC comprising or expressing at least one CAR described herein exhibits one or both of increased expression of favorable genes or decreased expression of unfavorable genes, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein exhibits increased production of ROS, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein exhibits metabolic reprogramming, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein exhibits induction of cell survival mechanisms, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a DC comprising or expressing at least one CAR described herein exhibits induction of cell death mechanisms, e.g., relative to a DC comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a DC comprising or expressing at least one CAR described herein exhibits one, two, three, four, or five of: increased resistance to phagocytic checkpoints, increased expression of chemokine receptors to aid in trafficking, increased expression of chemokines to recruit other immune cells, increased expression of ECM degrading enzymes (e.g., MMPs to degrade tumor ECM and/or exhibit anti fibrotic activity), and/or increased proliferation, e.g., relative to a DC without a CAR described herein. In some embodiments, a DC comprising or expressing at least one CAR described herein exhibits one, two, three, or four of: improved duration of CAR expression, improved stability of the CAR on the cell surface, increased level of CAR expression, and/or decreased background activity of the CAR, e.g., relative to a DC without a CAR described herein.

### Chimeric Antigen Receptors (CAR)

The term “chimeric antigen receptor” or “CAR,” as used herein, refers to an artificial cell surface receptor that is engineered to be expressed on an immune effector cell and specifically targets a cell and/or binds an antigen. CARs may be used, for example, as a therapy with adoptive cell transfer. For example, in some embodiments, immune cells (e.g., stem cells, macrophages, monocytes, and/or dendritic cells) are removed from a patient (e.g., from blood, tumor or ascites fluid) and modified so that they express a receptor specific to a particular form of antigen. In some embodiments, such modified immune cells are then reintroduced to the same or a different subject as a therapeutics. In some embodiments, CARs have been expressed with specificity to an antigen, for example, a tumor associated antigen. In some embodiments, a CAR comprises an extracellular domain, a transmembrane domain and an intracellular domain.

In some embodiments, a modified immune cell, for example, a modified stem cell, macrophage, monocyte, or dendritic cell, is generated by expressing a CAR therein. In some embodiments, an immune cell comprises a CAR comprising an extracellular domain, a transmembrane domain, and an intracellular domain, wherein the immune cell comprises a stem cell, macrophage, monocyte, or dendritic cell.

In some embodiments, a CAR may further comprise one or more of: one or more extracellular leader domains, one or more extracellular hinge domains and one or more intracellular co-stimulatory domains.

In some embodiments, a CAR comprises a spacer domain or hinge between an extracellular domain and a transmembrane domain (i.e., an extracellular hinge domain). In some embodiments, a CAR comprises a spacer domain or hinge between an intracellular domain and a transmembrane domain (i.e., an intracellular hinge domain). As used herein, the term “spacer domain” or “hinge” refers to any oligo- or polypeptide that functions to link a transmembrane domain to either an extracellular domain or to an intracellular domain in a polypeptide chain. In some embodiments, a spacer domain or hinge may comprise up to 300 amino acids, preferably 10 to 100 amino acids and most preferably 25 to 50 amino acids. In some embodiments, a short oligo- or polypeptide linker, preferably between 2 and 10 amino acids in length, may form a linkage between a transmembrane domain and an intracellular domain of a CAR. An example of a linker includes a glycine-serine doublet.

In some embodiments, an immune cell (e.g., a stem cell, macrophage, monocyte, or dendritic cell) comprising a CAR may comprise one or more control systems including, but not limited to: a safety switch (e.g., an on switch, and off switch, a suicide switch), and/or a logic gate (for example an AND gate (e.g., two or more CARs, each of which lacks one or more signaling domains such that activation of both/all CARs is required for full immune cell (e.g., stem cell, macrophage, monocyte, or dendritic cell) activation or function), an OR gate (e.g., two or more CARs, each with an intracellular domain such as CD3C and a co-stimulatory domain), and/or a NOT gate (e.g., two or more CARs, one of which includes an inhibitory domain that antagonizes the function of the other CAR[s])).

The present disclosure also provides immune cells (e.g., stem cells, macrophages, monocytes, or dendritic cells) comprising a nucleic acid (e.g., an isolated nucleic acid) encoding a CAR, wherein the nucleic acid comprises a nucleic acid sequence encoding an extracellular domain, a nucleic acid sequence encoding a transmembrane domain and a nucleic acid sequence encoding an intracellular domain, wherein the cell is a stem cell, macrophage, monocyte or dendritic cell that expresses the CAR.

In some embodiments, a CAR comprises an extracellular domain that is operably linked to another domain of the CAR, such as a transmembrane domain or an intracellular domain, for expression in an immune cell. In some embodiments, a nucleic acid encoding an extracellular domain is operably linked to a nucleic acid encoding a transmembrane domain and the nucleic acid encoding the transmembrane domain is operably linked to a nucleic acid encoding an intracellular domain.

In some embodiments, an effector activity of an immune cell comprising a CAR is directed against a target cell comprising an antigen that specifically binds an antigen binding domain of the CAR. In some embodiments, a targeted effector activity directed against a target cell is or comprises phagocytosis, targeted cellular cytotoxicity, antigen presentation, or cytokine secretion.

In some embodiments, a CAR described herein comprises at least one domain (e.g., an extracellular domain, a transmembrane domain, and/or an intracellular domain) that inhibits anti-phagocytic signaling in an immune cell described herein (e.g., a stem cell, macrophage, monocyte, or dendritic cell). In some embodiments, a CAR described herein improves effector activity of an immune cell described herein (e.g., a stem cell, macrophage, monocyte, or dendritic cell), e.g., by enhancing inhibition of CD47 and/or SIRPa activity. In some embodiments, a CAR described herein binds CD47, e.g., and serves as a dominant negative receptor, inhibiting SIRPa activity (e.g., a CD47 sink). In some embodiments, a CAR described herein that binds SIRPα, e.g., comprises an activating receptor (e.g., comprises a CD3z intracellular domain). In some embodiments, a CAR described herein inhibits at least one interaction of CD47 and SIRPα. In some embodiments, a CAR is or comprises a phagocytic logic gate.

In some embodiments, an immune cell described herein (e.g., comprising at least one CAR described herein) comprises or expresses at least one variant or fragment of: SIRPα (e.g., a dominant negative SIRPa or a high-affinity engineered variant of SIRPa (e.g., CV1)), 5F9 scFv, B6H12 scFv (e.g., a humanized B6H12 scFv), PD1 (e.g., a dominant negative PD1 or HAC-I), anti-PD1 scFv (e.g., E27 or durvalumab), Siglec-10, Siglec-9, Siglec-11, and/or SHP-1. In some embodiments, a variant or fragment comprises a mutated intracellular domain. In some embodiments, a variant or fragment does not comprise or express at least one intracellular domain (e.g., an immune cell comprises or expresses an anti-CD47 scFv, CD8 hinge domain, and CD8 transmembrane). In some embodiments, an immune cell described herein (e.g., comprising or expressing at least one CAR described herein) comprises a dominant negative receptor, e.g., blocking an inhibitory checkpoint.

In some embodiments, a payload comprising a CAR described herein further comprises a cleavage peptide (e.g., a P2A, F2A, E2A and/or T2A peptide) and/or at least one second CAR comprising at least one inhibitory domain of anti-phagocytic signaling. In some embodiments, at least one second CAR comprises a SIRPa (e.g., a high-affinity engineered variant of SIRPa (e.g., CV1)), 5F9 scFv, B6H12 scFv (e.g., a humanized B6H12 scFv), or a CD47 binding extracellular domain or a fragment thereof. In some embodiments, at least one second CAR comprises a SIRPa transmembrane domain or a fragment thereof. In certain embodiments, a second CAR further comprises a hinge domain (e.g., a CD8 hinge domain). In certain embodiments, at least one second CAR comprises: (i) a leader sequence (e.g., a CD8 leader); ii) an extracellular domain (e.g., a SIRPα, CV1, 5F9 scFv, or B6H12 scFv (e.g., a humanized B6H12 scFv) extracellular domain); and ii) a transmembrane domain (e.g., a SIRPα transmembrane domain). In some embodiments, a payload comprising a CAR described herein further comprises a cleavage peptide (e.g., a P2A peptide) and at least one marker protein (e.g., CD20 or a fragment thereof, CD19 or a fragment thereof, NGFR or a fragment thereof, a synthetic peptide, and/or a fluorescent protein).

In some embodiments, an immune cell described herein (e.g., comprising or expressing at least one CAR described herein) comprises or expresses one or more phosphatase dead domains (e.g. a phosphatase dead Shp1, phosphatase dead 72-5ptase (INPP5E), phosphatase dead Shp2, and/or phosphatase dead SHIP-1 domain) and/or a constitutively active kinase domain (e.g., a constitutively active LYN domain). In some embodiments, a payload comprising a CAR described herein further comprises a cleavage peptide (e.g., a P2A, F2A, E2A and/or T2A peptide) and one or more phosphatase dead domains (e.g. a phosphatase dead Shp1, phosphatase dead 72-5ptase (INPPSE), phosphatase dead Shp2, and/or phosphatase dead SHIP-1 domain) and/or a constitutively active kinase domain (e.g., a constitutively active LYN domain).

In some embodiments, a CAR of the present disclosure binds to mesothelin and comprises an amino acid sequence that is at least 80% identical to a sequence selected from Table 2. In some embodiments, a CAR of the present disclosure binds to mesothelin and comprises an amino acid sequence that is at least 85% identical to a sequence selected from Table 2. In some embodiments, a CAR of the present disclosure binds to mesothelin and comprises an amino acid sequence that is at least 90% identical to a sequence selected from Table 2. In some embodiments, a CAR of the present disclosure binds to mesothelin and comprises an amino acid sequence that is at least 95% identical to a sequence selected from Table 2. In some embodiments, a CAR of the present disclosure binds to mesothelin and comprises an amino acid sequence that is at least 96% identical to a sequence selected from Table 2. In some embodiments, a CAR of the present disclosure binds to mesothelin and comprises an amino acid sequence that is at least 97% identical to a sequence selected from Table 2. In some embodiments, a CAR of the present disclosure binds to mesothelin and comprises an amino acid sequence that is at least 98% identical to a sequence selected from Table 2. In some embodiments, a CAR of the present disclosure binds to mesothelin and comprises an amino acid sequence that is at least 99% identical to a sequence selected from Table 2. In some embodiments, a CAR of the present disclosure binds to mesothelin and comprises an amino acid sequence that is identical to a sequence selected from Table 2.

In some embodiments, a CAR of the present disclosure binds to mesothelin and comprises an amino acid sequence that differs from a sequence selected from Table 2 by no more than 50 substitutions, additions, or deletions. In some embodiments, a CAR of the present disclosure binds to mesothelin and comprises an amino acid sequence that differs from a sequence selected from Table 2 by no more than 40 substitutions, additions, or deletions. In some embodiments, a CAR of the present disclosure binds to mesothelin and comprises an amino acid sequence that differs from a sequence selected from Table 2 by no more than 30 substitutions, additions, or deletions. In some embodiments, a CAR of the present disclosure binds to mesothelin and comprises an amino acid sequence that differs from a sequence selected from Table 2 by no more than 20 substitutions, additions, or deletions. In some embodiments, a CAR of the present disclosure binds to mesothelin and comprises an amino acid sequence that differs from a sequence selected from Table 2 by no more than 10 substitutions, additions, or deletions. In some embodiments, a CAR of the present disclosure binds to mesothelin and comprises an amino acid sequence that differs from a sequence selected from Table 2 by no more than 5 substitutions, additions, or deletions. In some embodiments, a CAR of the present disclosure binds to mesothelin and comprises an amino acid sequence that differs from a sequence selected from Table 2 by no more than 4 substitutions, additions, or deletions. In some embodiments, a CAR of the present disclosure binds to mesothelin and comprises an amino acid sequence that differs from a sequence selected from Table 2 by no more than 3 substitutions, additions, or deletions. In some embodiments, a CAR of the present disclosure binds to mesothelin and comprises an amino acid sequence that differs from a sequence selected from Table 2 by no more than 2 substitutions, additions, or deletions. In some embodiments, a CAR of the present disclosure binds to mesothelin and comprises an amino acid sequence that differs from a sequence selected from Table 2 by no more than 1 substitution, addition, or deletion. In some embodiments, a CAR of the present disclosure binds to mesothelin and comprises an amino acid sequence that does not have any substitutions, additions, or deletions relative to a sequence selected from Table 2.

### CAR Extracellular Domains

The present disclosure provides chimeric antigen receptors (CAR) comprising one or more extracellular domains. In some embodiments, a CAR extracellular domain comprises an Fc receptor (FcR) extracellular domain. In some embodiments, a CAR extracellular domain comprises a toll-like receptor (TLR) extracellular domain. In some embodiments, a CAR extracellular domain comprises a leader domain. In some embodiments, a CAR extracellular domain comprises an antigen binding domain. In some embodiments, a CAR extracellular domain comprises a hinge domain. In some embodiments, a CAR extracellular domain comprises one or more of an FcR extracellular domain, a TLR extracellular domain, a leader domain, an antigen binding domain and a hinge domain. In some embodiments, a CAR extracellular domain may be a domain that is endogenous to a particular immune cell type (e.g., a modified immune cell as provided herein). In some embodiments, a CAR extracellular domain may be a domain that is not endogenous to a particular immune cell type (e.g., a modified immune cell as provided herein).

### FcR Extracellular Domains

In some embodiments, an FcR extracellular domain comprises a full-length FcR extracellular domain. In some embodiments, an FcR extracellular domain comprises a portion of a full-length FcR extracellular domain. In some embodiments, an FcR extracellular domain (or portion thereof) is or comprises a human FcR extracellular domain. In some embodiments, an FcR extracellular domain may be a domain that is endogenous to a particular immune cell type (e.g., a modified immune cell as provided herein). In some embodiments, an FcR extracellular domain may be a domain that is not endogenous to a particular immune cell type (e.g., a modified immune cell as provided herein). In some embodiments, an FcR extracellular domain comprises an FcRγ, CD64 (FcγRI), CD32a (FcγRIIa), CD32b (FcγRIIb), CD32c, CD16a (FcγRIIIa), CD16b (FcγRIIIb), FcεRI, FcεRII, or FcαRI (CD89) domain.

### TLR Extracellular Domains

In some embodiments, a TLR extracellular domain comprises a full-length TLR extracellular domain. In some embodiments, a TLR extracellular domain comprises a portion of a full-length TLR extracellular domain. In some embodiments, a TLR extracellular domain (or portion thereof) is or comprises a human TLR extracellular domain. In some embodiments, a TLR extracellular domain may be a domain that is endogenous to a particular immune cell type (e.g., a modified immune cell as provided herein). In some embodiments, a TLR extracellular domain may be a domain that is not endogenous to a particular immune cell type (e.g., a modified immune cell as provided herein). In some embodiments, a TLR extracellular domain comprises a TLR1, TLR2, TLR3, TLR4, TLR5, TLR6, TLR7, TLR8, or TLR9 domain.

### Leader Domains

In some embodiments, a CAR comprises one or more extracellular leader domains. In some embodiments, a nucleic acid encoding a CAR comprises a nucleic acid sequence encoding an extracellular leader domain, but the extracellular leader domain is cleaved from the CAR before the CAR is expressed in an immune cell. In some embodiments, an extracellular leader domain is or comprises a human extracellular leader domain. In some embodiments, an extracellular leader domain may be a domain that is endogenous to a particular immune cell type (e.g., a modified immune cell as provided herein). In some embodiments, an extracellular leader domain may be a domain that is not endogenous to a particular immune cell type (e.g., a modified immune cell as provided herein). In some embodiments, an extracellular leader domain comprises a CD8 extracellular leader domain. In some embodiments, an extracellular leader domain comprises a leader domain from a stimulatory or co-stimulatory domain (e.g., a TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88 domain).

### Antigen Binding Domains

In some embodiments, a CAR comprises an antigen binding domain that binds to an antigen, for example, on a target cell. In some embodiments, a CAR comprises an antigen binding domain that binds to an antigen associated with cancer cells. In some embodiments, a CAR antigen binding domain recognizes an antigen that acts as a cell surface marker on a target cell associated with a particular disease state.

In some embodiments, a CAR antigen binding domain binds to a tumor antigen, such as an antigen that is specific for a tumor or cancer of interest. In some embodiments a tumor antigen comprises one or more antigenic cancer epitopes. In some embodiments, a tumor antigen comprises mesothelin.

In some embodiments, a CAR antigen binding domain binds to mesothelin and comprises an amino acid sequence that is at least 80% identical to a sequence selected from Table 3. In some embodiments, a CAR antigen binding domain binds to mesothelin and comprises an amino acid sequence that is at least 85% identical to a sequence selected from Table 3. In some embodiments, a CAR antigen binding domain binds to mesothelin and comprises an amino acid sequence that is at least 90% identical to a sequence selected from Table 3. In some embodiments, a CAR antigen binding domain binds to mesothelin and comprises an amino acid sequence that is at least 95% identical to a sequence selected from Table 3. In some embodiments, a CAR antigen binding domain binds to mesothelin and comprises an amino acid sequence that is at least 96% identical to a sequence selected from Table 3. In some embodiments, a CAR antigen binding domain binds to mesothelin and comprises an amino acid sequence that is at least 97% identical to a sequence selected from Table 3. In some embodiments, a CAR antigen binding domain binds to mesothelin and comprises an amino acid sequence that is at least 98% identical to a sequence selected from Table 3. In some embodiments, a CAR antigen binding domain binds to mesothelin and comprises an amino acid sequence that is at least 99% identical to a sequence selected from Table 3. In some embodiments, a CAR antigen binding domain binds to mesothelin and comprises an amino acid sequence identical to a sequence selected from Table 3.

In some embodiments, a CAR antigen binding domain binds to mesothelin and comprises an amino acid sequence that differs from a sequence selected from Table 3 by no more than 10 substitutions, additions, or deletions. In some embodiments, a CAR antigen binding domain binds to mesothelin and comprises an amino acid sequence that differs from a sequence selected from Table 3 by no more than 9 substitutions, additions, or deletions. In some embodiments, a CAR antigen binding domain binds to mesothelin and comprises an amino acid sequence that differs from a sequence selected from Table 3 by no more than 8 substitutions, additions, or deletions. In some embodiments, a CAR antigen binding domain binds to mesothelin and comprises an amino acid sequence that differs from a sequence selected from Table 3 by no more than 7 substitutions, additions, or deletions. In some embodiments, a CAR antigen binding domain binds to mesothelin and comprises an amino acid sequence that differs from a sequence selected from Table 3 by no more than 6 substitutions, additions, or deletions. In some embodiments, a CAR antigen binding domain binds to mesothelin and comprises an amino acid sequence that differs from a sequence selected from Table 3 by no more than 5 substitutions, additions, or deletions. In some embodiments, a CAR antigen binding domain binds to mesothelin and comprises an amino acid sequence that differs from a sequence selected from Table 3 by no more than 4 substitutions, additions, or deletions. In some embodiments, a CAR antigen binding domain binds to mesothelin and comprises an amino acid sequence that differs from a sequence selected from Table 3 by no more than 3 substitutions, additions, or deletions. In some embodiments, a CAR antigen binding domain binds to mesothelin and comprises an amino acid sequence that differs from a sequence selected from Table 3 by no more than 2 substitutions, additions, or deletions. In some embodiments, a CAR antigen binding domain binds to mesothelin and comprises an amino acid sequence that differs from a sequence selected from Table 3 by no more than 1 substitution, addition, or deletion. In some embodiments, a CAR antigen binding domain binds to mesothelin and comprises an amino acid sequence that does not have any substitutions, additions, or deletions relative to a sequence selected from Table 3.

In some embodiments, a CAR antigen binding domain comprises any domain that binds to an antigen. In some embodiments, a CAR antigen binding domain is or comprises a monoclonal antibody, a polyclonal antibody, a synthetic antibody, a human antibody, a humanized antibody, a non-human antibody, or any fragment thereof, for example an scFv. In some embodiments, a CAR antigen binding domain is or comprises an aptamer, a darpin, a centyrin, a naturally occurring or synthetic receptor, an affibody, a nanobody, or other engineered protein recognition molecule. In some embodiments, a CAR antigen binding domain is or comprises a mammalian antibody or a fragment thereof. In some embodiments, a CAR antigen binding domain is derived, in whole or in part, from the same species in which the CAR will ultimately be used. For example, for use in humans, an antigen binding domain of a CAR comprises a human antibody, a humanized antibody, or a fragment thereof (e.g. a scFv). In some embodiments, a CAR antigen binding domain may be a domain that is endogenous to a particular immune cell type (e.g., a modified immune cell as provided herein). In some embodiments, a CAR antigen binding domain may be a domain that is not endogenous to a particular immune cell type (e.g., a modified immune cell as provided herein).

In some embodiments, a CAR comprises one or more antigen binding domains. In some embodiments, a CAR comprises two or more antigen binding domains. In some embodiments, a CAR is a bispecific CAR. In some embodiments, an immune cell comprises two or more different CARs comprising one or more antigen binding domains. In some embodiments, an immune cell comprising a bispecific CAR and/or comprising two or more different CARs comprising one or more antigen binding domains can reduce off-target and/or on-target off-tissue effects by requiring that two antigens are present. In some embodiments, an immune cell comprises a bispecific CAR and/or comprises two or more different CARs comprising one or more antigen binding domains, wherein the CARs provide distinct signals that in isolation are insufficient to mediate activation of the modified cell, but are synergistic together, stimulating activation of the modified cell. In some embodiments, such a construct may be referred to as an ‘AND’ logic gate.

In some embodiments, an immune cell comprising a bispecific CAR and/or comprising two or more different CARs comprising one or more antigen binding domains can reduce off-target and/or on-target off-tissue effects by requiring that one antigen is present and a second, normal protein antigen is absent before the cell's activity is stimulated. In some embodiments, such a construct may be referred to as a ‘NOT’ logic gate. In contrast to AND gates, NOT gated CAR-modified cells are activated by binding to a single antigen. However, the binding of a second receptor to the second antigen functions to override the activating signal being perpetuated through the CAR. Typically, such an inhibitory receptor would be targeted against an antigen that is abundantly expressed in a normal tissue but is absent in tumor tissue.

### Hinge Domains

In some embodiments, a CAR comprises one or more extracellular hinge domains. In some embodiments, a CAR extracellular hinge domain is or comprises a human extracellular hinge domain. In some embodiments, a CAR extracellular hinge domain may be a domain that is endogenous to a particular immune cell type (e.g., a modified immune cell as provided herein). In some embodiments, a CAR extracellular hinge domain may be a domain that is not endogenous to a particular immune cell type (e.g., a modified immune cell as provided herein). In some embodiments, one or more CAR extracellular hinge domains comprise a CD8a extracellular hinge domain, a DNGR-1 extracellular hinge domain, a Dectin-1 extracellular hinge domain, or an IgG4 or a CD28 extracellular hinge domain. In some embodiments, a CAR extracellular hinge domain optimizes the physicochemical parameters of a CAR, e.g., optimal size relative to tumor antigen (e.g., allowing for exclusion of inhibitory molecules), optimal flexibility, optimal protein folding, optimal protein stability, optimal binding, optimal homodimerization, and/or lack of homodimerization.

### CAR Transmembrane Domains

In some embodiments, a CAR comprises a transmembrane domain, for example, that connects an extracellular domain to an intracellular domain. In some embodiments, a CAR transmembrane domain is naturally associated with one or more other domain(s) of a CAR. In some embodiments, a CAR transmembrane domain can be modified to avoid binding to transmembrane domains of other surface membrane proteins, in order to minimize interactions with other members of a receptor complex. In some embodiments, a CAR transmembrane domain may be derived either from a naturally-occurring or from a synthetic source. In some embodiments a CAR transmembrane domain is derived from a naturally-occurring membrane-bound or transmembrane protein. In some embodiments, a CAR transmembrane domain is or comprises a human transmembrane domain. In some embodiments, a CAR transmembrane domain may be a domain that is endogenous to a particular immune cell type (e.g., a modified immune cell as provided herein). In some embodiments, a CAR transmembrane domain may be a domain that is not endogenous to a particular immune cell type (e.g., a modified immune cell as provided herein). In some embodiments, a CAR transmembrane domain comprises a CD8, CD8a, CD64, CD32a, CD32c, CD16a, TRL1, TLR2, TLR3, TRL4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88, CD3-zeta, Dectin-1, DNGR1, SLAMF7, FcR γ, V/I/LxYxxL/V, SIRPα, CD45, Siglec-10, PD1, SHP-1, SHP-2, KIR-2DL, KIR-3DL, NKG2A, CD170, CD33, BTLA, CD32b, SIRPβ, CD22, PIR-B, LILRB1, CD36, or Syk transmembrane domain.

### FcR Transmembrane Domains

In some embodiments, an FcR transmembrane domain comprises a full-length FcR transmembrane domain. In some embodiments, an FcR transmembrane domain comprises a portion of a full-length FcR transmembrane domain. In some embodiments, an FcR transmembrane domain is or comprises a human FcR transmembrane domain, or portion thereof. In some embodiments, an FcR transmembrane domain may be a domain that is endogenous to a particular immune cell type (e.g., a modified immune cell as provided herein). In some embodiments, an FcR transmembrane domain may be a domain that is not endogenous to a particular immune cell type (e.g., a modified immune cell as provided herein). In some embodiments, an FcR transmembrane domain comprises an FcRγ, CD64 (FcγRI), CD32a (FcγRIIa), CD32b (FcγRIIb), CD32c, CD16a (FcγRIIIa), CD16b (FcγRIIIb), FcεRI, FcεRII, or FcαRI (CD89) domain.

### TLR Transmembrane Domains

In some embodiments, a TLR transmembrane domain comprises a full-length TLR transmembrane domain. In some embodiments, a TLR transmembrane domain comprises a portion of a full-length TLR transmembrane domain. In some embodiments, a TLR transmembrane domain is or comprises a human TLR transmembrane domain, or portion thereof. In some embodiments, a TLR transmembrane domain may be a domain that is endogenous to a particular immune cell type (e.g., a modified immune cell as provided herein). In some embodiments, a TLR transmembrane domain may be a domain that is not endogenous to a particular immune cell type (e.g., a modified immune cell as provided herein). In some embodiments, a TLR transmembrane domain comprises a TLR1, TLR2, TLR3, TLR4, TLR5, TLR6, TLR7, TLR8, or TLR9 domain.

### CAR Intracellular Domains

In some embodiments, a CAR comprises one or more intracellular domains. In some embodiments, a CAR intracellular domain is or comprises a human intracellular domain, or portion thereof. In some embodiments, a CAR intracellular domain may be a domain that is endogenous to a particular immune cell type (e.g., a modified immune cell as provided herein). In some embodiments, a CAR intracellular domain may be a domain that is not endogenous to a particular immune cell type (e.g., a modified immune cell as provided herein). In some embodiments, a CAR intracellular domain and/or other cytoplasmic domain of a CAR is responsible for activation of the cell in which the CAR is expressed (e.g., a modified immune cell as provided herein). In some embodiments, a CAR intracellular domain of a CAR is responsible for signal activation and/or transduction in an immune cell comprising said CAR.

In some embodiments, a CAR intracellular domain of a CAR includes at least one domain responsible for signal activation and/or transduction. In some embodiments, a CAR intracellular domain is or comprises at least one of a co-stimulatory molecule and a signaling domain. In some embodiments, a CAR intracellular domain of a CAR comprises dual signaling domains. In some embodiments, a CAR intracellular domain of a CAR comprises more than two signaling domains.

In some embodiments, a CAR intracellular domain comprises a cytoplasmic portion of a surface receptor. In some embodiments, a CAR intracellular domain comprises a co-stimulatory molecule. In some embodiments, a CAR intracellular domain comprises a molecule that acts to initiate signal transduction in an immune cell.

In some embodiments, an intracellular domain of a CAR includes any portion of one or more co-stimulatory molecules, such as at least one signaling domain from CD3ζ, Fc epsilon RI gamma chain, any derivative or variant thereof, any synthetic sequence thereof that has the same functional capability, and any combination thereof.

### FcR Intracellular Domains

In some embodiments, an FcR intracellular domain comprises a full-length FcR intracellular domain. In some embodiments, an FcR intracellular domain comprises a portion of a full-length FcR intracellular domain. In some embodiments, an FcR intracellular domain is or comprises a human FcR intracellular domain, or portion thereof. In some embodiments, an FcR intracellular domain may be a domain that is endogenous to a particular immune cell type (e.g., a modified immune cell as provided herein). In some embodiments, an FcR intracellular domain may be a domain that is not endogenous to a particular immune cell type (e.g., a modified immune cell as provided herein). In some embodiments, an FcR intracellular domain comprises an FcRγ, CD64 (FcγRI), CD32a (FcγRIIa), CD32b (FcγRIIb), CD32c, CD16a (FcγRIIIa), CD16b (FcγRIIIb), FcεRI, FcεRII, or FcαRI (CD89) domain.

### TLR Intracellular Domains

In some embodiments, a TLR intracellular domain comprises a full-length TLR intracellular domain. In some embodiments, a TLR intracellular domain comprises a portion of a full-length TLR intracellular domain. In some embodiments, a TLR intracellular domain is or comprises a human TLR intracellular domain, or portion thereof. In some embodiments, a TLR intracellular domain may be a domain that is endogenous to a particular immune cell type (e.g., a modified immune cell as provided herein). In some embodiments, a TLR intracellular domain may be a domain that is not endogenous to a particular immune cell type (e.g., a modified immune cell as provided herein). In some embodiments, a TLR intracellular domain comprises a TLR1, TLR2, TLR3, TLR4, TLR5, TLR6, TLR7, TLR8, or TLR9 domain.

### Signaling Domains

In some embodiments, a CAR comprises one or more intracellular signaling domains. In some embodiments, a CAR intracellular signaling domain is or comprises a human intracellular signaling domain, or portion thereof. In some embodiments, a CAR signaling domain may be a domain that is endogenous to a particular immune cell type (e.g., a modified immune cell as provided herein). In some embodiments, a CAR signaling domain may be a domain that is not endogenous to a particular immune cell type (e.g., a modified immune cell as provided herein).

In some embodiments, one or more CAR intracellular signaling domains comprise a CD3zeta (CD3Q), FcRγ, CD64, CD32a, CD32c, CD16a, CD40, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88, V/I/LxYxxL/V, SIRPα, CD45, Siglec-10, PD1, SHP-1, SHP-2, KIR-2DL, KIR-3DL, NKG2A, CD170, CD33, BTLA, CD32b, SIRPβ, CD22, PIR-B, LILRB1, Syk, 41BB ligand (41BBL; TNFSF9), CD27, OX40L, CD32b, CD11b, ITGAM, SLAMF7, CD206, CD163, CD209, GCSFR (CD114), RAGE, CD30, CD160, DR3, Fn14, HVEM, CD160, NGFR, RANK, TNFR2, TROY, XEDAR, TRIF, Dectin-2, or one or more cytokine receptor signaling domains (e.g., an IL1R, an IL2R, an IL3R, an IL4R, an IL5R, an IL6R, an IL7R, an IL8R, an IL9R, an IL10R, an IL11R, an IL12R, an IL13R, an IL14R, an IL15R, an IL17R, an IFNaR, an IFNgR, an TNFR, an CSF1R, an CSF2R, Dap10, CD36, Dectin-1, or ICOSL intracellular signaling domain).

In some embodiments, an intracellular domain of a CAR comprises dual signaling domains, such as 41BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination.

### Co-Stimulatory Domains

As used herein, a “co-stimulatory molecule” or “co-stimulatory domain” refers to a molecule in an immune cell that is used to heighten or dampen an initial stimulus. For example, pathogen-associated pattern recognition receptors, such as TLR or the CD47/SIRPa axis, are molecules on immune cells that, respectively, heighten or dampen an initial stimulus. In some embodiments, a CAR co-stimulatory domain comprises TCR, CD3 zeta (CD3ζ), CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combinations thereof.

In some embodiments, a CAR co-stimulatory domain may be a domain that is endogenous to a particular immune cell type (e.g., a modified immune cell as provided herein). In some embodiments, a CAR co-stimulatory domain may be a domain that is not endogenous to a particular immune cell type (e.g., a modified immune cell as provided herein).

As used herein, a “co-stimulatory signal” refers to a signal, which in combination with a primary signal, such as activation of a CAR on an immune cell, leads to activation of the immune cell.

### Cleavage Peptides

As used herein, a cleavage peptide refers to a peptide that can induce the cleaving of a recombinant protein in a cell. In some embodiments, a cleavage peptide is a 2A peptide. In some embodiments, a cleavage peptide is or comprises a P2A, F2A, E2A or T2A peptide. In some embodiments, a nucleic acid described herein comprises one or more nucleic acid sequences encoding one or more cleavage peptides. In some embodiments, a nucleic acid comprising a nucleic acid sequence encoding a cleavage peptide also comprises one or more nucleic acid sequences encoding one or more intracellular domains and one or more nucleic acid sequences comprising one or more peptide agents, wherein translation of the nucleic acid results in a protein comprising one or more intracellular domains separated from one or more peptide agents by a cleavage peptide. In some embodiments, a first promoter is operably linked to one or more nucleic acids encoding a CAR and a second promoter is operably linked to one or more nucleic acids encoding a peptide agent. In some embodiments, a nucleic acid sequence comprising a CAR, and optionally one or more peptide agents, further comprises an internal ribosome entry site (IRES) sequence. An IRES sequence may be any viral, chromosomal or artificially designed sequence that initiates cap-independent ribosome binding to mRNA facilitates the initiation of translation.

### CAR Peptide Agents

As used herein, a CAR peptide agent refers to a peptide co-expressed with a CAR in an immune cell. In some embodiments, a CAR peptide agent is co-expressed with a CAR to ensure stoichiometric balance and optimal signaling of a CAR. In some embodiments, a CAR peptide agent forms a homodimer with an identical peptide agent. In some embodiments, a CAR peptide agent forms a heterodimer with a different peptide agent. In some embodiments, a nucleic acid described herein comprises one or more nucleic acid sequences encoding one or more CAR peptide agents. In some embodiments, a CAR peptide agent is or comprises an FcR gamma chain.

In some embodiments, a CAR peptide agent comprises any peptide, protein, receptor, secreted antibody or a fragment thereof (e.g., an scFv, Fab, Fab′, F(ab′)2, Fc, or nanobody). In some embodiments, a CAR peptide agent comprises one or more cytokines (e.g., one or more of IL-1, IL-2, IL-6, IL-8, TNF-α, IFNα, IFNb, IFN-γ, GMCSF, or MCSF), CD40-L, dominant negative SIRPα, dominant negative PD1, dominant negative CD45, dominant negative SIGLEC 10, or dominant negative LILRB.

### Fc Receptors (FcR)

In some embodiments, a CAR comprises one or more antigen binding domains and an FcR extracellular domain, and/or the transmembrane domain of the CAR comprises an FcR transmembrane domain, and/or the intracellular domain of the CAR comprises an FcR intracellular domain. In some embodiments, a CAR comprises, from N-terminus to C-terminus, one or more extracellular binding domains, an FcR extracellular domain, an FcR transmembrane domain, and an FcR intracellular domain. In some embodiments, one or more of the FcR extracellular domain, the FcR transmembrane domain and the FcR intracellular domain is or comprises a human FcR domain. In some embodiments, an FcR extracellular domain, an FcR transmembrane domain and an FcR intracellular domain together comprise a full-length FcR. In some embodiments, an FcR extracellular domain, an FcR transmembrane domain and an FcR intracellular domain together comprise a portion of a full-length FcR. In some embodiments, an FcR extracellular domain comprises a portion of a full-length FcR extracellular domain. In some embodiments, an FcR transmembrane domain comprises a portion of a full-length FcR transmembrane domain. In some embodiments, an FcR intracellular domain comprises a portion of a full-length FcR intracellular domain.

### Toll-Like Antigen Receptors (TLR)

In some embodiments, a CAR comprises one or more antigen binding domains and a toll-like receptor (TLR) extracellular domain and/or the transmembrane domain of the CAR comprises a TLR transmembrane domain and/or the intracellular domain of the CAR comprises a TLR intracellular domain. In some embodiments, a CAR comprises, from N-terminus to C-terminus, one or more extracellular binding domains, a TLR extracellular domain, a TLR transmembrane domain, and a TLR intracellular domain. In some embodiments, one or more of the TLR extracellular domain, the TLR transmembrane domain and the TLR intracellular domain is or comprises a human TLR domain. In some embodiments, a TLR extracellular domain, a TLR transmembrane domain and a TLR intracellular domain together comprise a full-length TLR. In some embodiments, a TLR extracellular domain, a TLR transmembrane domain and a TLR intracellular domain together comprise portion of a full-length TLR. In some embodiments, a TLR extracellular domain comprises a portion of a full-length TLR extracellular domain. In some embodiments, a TLR transmembrane domain comprises a portion of a full-length TLR transmembrane domain. In some embodiments, a TLR intracellular domain comprises a portion of a full-length TLR intracellular domain.

### Methods of Immune Cell Modification

The present disclosure provides, among other things, methods for modifying an immune cell (e.g., a stem cell, monocyte, macrophage, or dendritic cell) comprising delivering to the immune cell a nucleic acid construct comprising one or more nucleic acids encoding one or more CARs described herein. Methods can comprise delivering to an immune cell (e.g., a stem cell, monocyte, macrophage, or dendritic cell), a nucleic acid construct comprising one or more nucleic acids encoding: at least one extracellular domain described herein, at least one transmembrane domain described herein, and at least one intracellular domain described herein.

In some embodiments, the present disclosure provides methods of producing a modified immune cell (e.g., a stem cell, monocyte, macrophage, or dendritic cell) in a subject comprising administering to a subject a composition as described herein comprising: (a) one or more nucleic acid molecules, wherein at least a portion of one or more nucleic acid molecules encodes a CAR and/or CAR peptide agent, and (b) a delivery vehicle. Accordingly, in some embodiments, following administration of the composition one or more nucleic acid molecules are translated in an immune cell (e.g., stem cell, macrophage, monocyte, or dendritic cell) to produce a modified immune cell comprising the CAR and/or CAR peptide agent. In some embodiments, the modified immune cell comprising the CAR and/or CAR peptide agent possesses targeted effector activity.

### Delivery Methods

A nucleic acid construct comprising one or more nucleic acid sequences encoding at least one CAR described herein can be introduced into an immune cell (e.g., a stem cell, macrophage, monocyte, or dendritic cell) by physical, chemical, or biological methods. In some embodiments, the present disclosure provides methods for modifying an immune cell comprising producing a modified immune cell (e.g., a stem cell, monocyte, macrophage, or dendritic cell) ex vivo. In some embodiments, the present disclosure provides methods for modifying an immune cell comprising producing a modified immune cell (e.g., a stem cell, monocyte, macrophage, or dendritic cell) in a subject (i.e., in vivo).

Physical methods for introducing a nucleic acid construct described herein into an immune cell (e.g., a stem cell, macrophage, monocyte, or dendritic cell) can comprise electroporation, calcium phosphate precipitation, lipofection, particle bombardment, microinjection, or a combination thereof. A nucleic acid construct can be introduced into immune cells using commercially available methods, including electroporation (Amaxa Nucleofector-II® (Amaxa Biosystems, Cologne, Germany), ECM 830 BTX (Harvard Instruments, Boston, Mass.) Gene Pulser II® (BioRad, Denver, Colo.), or Multiporator® (Eppendort, Hamburg Germany)). A nucleic acid construct can also be introduced into immune cells using mRNA transfection, e.g., cationic liposome-mediated transfection, lipofection, polymer encapsulation, peptide-mediated transfection, or biolistic particle delivery systems, such as “gene guns” (See, e.g., Nishikawa, et al. Hum Gene Ther., 12(8):861-70 (2001), which is hereby incorporated by reference in its entirety).

Biological methods for introducing a nucleic acid construct described herein into an immune cell (e.g., a stem cell, macrophage, monocyte, or dendritic cell) include use of DNA and RNA vectors. In one embodiment, a vector comprises a plasmid vector, a viral vector, a transposon, a retrotransposon (e.g., piggyback, sleeping beauty), a site directed insertion vector (e.g., CRISPR, Zn finger nucleases, TALEN), suicide expression vector, or another vector known in the art. Viral vectors, and especially retroviral vectors, have become widely used for inserting genes into mammalian cells (e.g., human cells). Viral vectors can also be derived from lentivirus, poxviruses, herpes simplex virus I, adenoviruses (e.g. Ad5f35), or adeno-associated viruses (See, e.g., U.S. Pat. Nos. 5,350,674 and 5,585,362, which are hereby incorporated by reference in their entirety). Retroviral vectors, such as lentivirus, are suitable tools to achieve long-term gene transfer that allow for long-term, stable integration of a transgene and its propagation in daughter cells. In some embodiments, a lentiviral vector is packaged with a VPX protein (e.g., as described in International Publication No. WO 2017/044487, which is hereby incorporated by reference in its entirety). In some embodiments, VPX comprises a virion-associated protein (e.g., an accessory protein for viral replication). In some embodiments, a VPX protein is encoded by human immunodeficiency virus type 2 (HIV-2). In some embodiments, a VPX protein is encoded by simian immunodeficiency virus (SIV). In some embodiments, an immune cell described herein (e.g., a stem cell, macrophage, monocyte, or dendritic cell) is transfected with a lentiviral vector packaged with a VPX protein. In some embodiments, VPX inhibits at least one antiviral factor of an immune cell described herein (e.g., a stem cell, macrophage, monocyte, or dendritic cell). In some embodiments, a lentiviral vector packaged with a VPX protein exhibits increased transfection efficiency of an immune cell described herein (e.g., a stem cell, macrophage, monocyte, or dendritic cell), e.g., relative to a lentiviral vector not packaged with a VPX protein. In some embodiments, an immune cell described herein (e.g., a stem cell, macrophage, monocyte, or dendritic cell) is one or both of electroporated or transfected with at least one VPX mRNA prior to transfection with a viral vector (e.g., an adenoviral vector, e.g., an Ad2 vector or an Ad5 vector (e.g., Ad5f35 adenoviral vector, e.g., a helper-dependent Ad5F35 adenoviral vector)).

Chemical means for introducing a nucleic acid construct described herein into an immune cell (e.g., a stem cell, macrophage, monocyte, or dendritic cell) include colloidal dispersion systems, macromolecule complexes, nanocapsules, microspheres, beads, and lipid-based systems (e.g., oil-in-water emulsions, micelles, mixed micelles, nanoparticles, liposomes, and lipofectamine-nucleic acid complexes).

An exemplary system for delivery of a nucleic acid construct described herein is a lipid-based system. A nucleic acid construct described herein may be encapsulated in an aqueous interior of a liposome, interspersed within a lipid bilayer, attached to a liposome via a linking molecule, attached to a lipid nanoparticle (LNP) via a linking molecule, entrapped in a liposome, entrapped in an LNP, complexed with a liposome, complexed with an LNP, dispersed in a solution or suspension comprising a lipid, mixed with a lipid, complexed with a micelle, or otherwise associated with a lipid. Lipids for use in methods described herein may be naturally occurring or synthetic lipids. Lipids can also be obtained from commercial sources. For example, dimyristyl phosphatidylcholine can be obtained from Sigma (St. Louis, MO); dicetyl phosphate can be obtained from K & K Laboratories (Plainview, NY); cholesterol can be obtained from Calbiochem-Behring; and dimyristyl phosphatidylglycerol can be obtained from Avanti Polar Lipids, Inc. (Birmingham, AL.). Stock solutions of lipids in chloroform or chloroform/methanol can be stored at about −20 °C. In some embodiments, a lipid-based system may comprise one or more lipids that facilitate targeting of the composition to a desired cell type or cell types (e.g., stem cells, monocytes, macrophages, or dendritic cells). In some embodiments, a delivery vehicle allows a composition to be preferentially taken up (e.g. endocytosed, phagocytosed) by an immune cell (e.g., stem cell, monocyte, macrophage, or dendritic cell) relative to a composition that does not comprise the delivery vehicle.

### Targeting Moieties

In some embodiments, a delivery vehicle may comprise one or more targeting moieties. In some embodiments, a targeting moiety may facilitate passive targeting of a composition to a desired target. In some embodiments, a targeting moiety may facilitate active targeting of a composition to a desired target.

In some embodiments, a targeting moiety may be or comprise one of more of an antibody (e.g., a monoclonal antibody, a polyclonal antibody, a synthetic antibody, a human antibody, a humanized antibody, a non-human antibody) or any fragment thereof, for example an scFv, an aptamer, a darpin, a centyrin, a naturally occurring or synthetic receptor, an affibody, or other engineered protein recognition molecule, for example, to bind to one or more of CD14, CD11b, CD163, CD206, CD33, CD209. In some embodiments, a targeting moiety may be or comprise a small molecule.

In some embodiments, a targeting moiety may be or comprise a particular lipid or combination of hydrophobic entities, for example, present in or forming an exterior surface of a liposome or lipid nanoparticle (e.g., for targeting to a particular cell type or cell types).

### Nucleic Acid Molecules

In some embodiments of the present disclosure, one or more nucleic acid molecules are or comprise DNA. In some embodiments of the present disclosure, one or more nucleic acid molecules are or comprise messenger RNA (mRNA). In some embodiments, mRNA according to the present disclosure may be synthesized as unmodified or modified mRNA. Typically, mRNAs are modified to enhance stability. Modifications of mRNA can include, for example, modifications of the nucleotides of the RNA. A modified mRNA according to the present disclosure can thus include, for example, backbone modifications, sugar modifications or base modifications. In some embodiments, a step of modifying an mRNA comprises causing the mRNA to include a modified nucleotide, an alteration to the 5′ or 3′ untranslated region (UTR), a cap structure, and/or a poly(A) tail.

In some embodiments, mRNAs of the present disclosure (e.g., mRNAs encoding one or more CARs described herein) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which comprises replacing the phosphodiester linkage by other anionic, cationic or neutral groups.

In some embodiments, mRNAs of the present disclosure (e.g., mRNAs encoding one or more CARs described herein) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, mRNAs of the present disclosure (e.g., mRNAs encoding one or more CARs described herein) comprise modified nucleotide comprising pseudouridine (PsU), 5-methoxyuridine (5moU), 5-methylcytidine/pseudouridine (5meC PsU), N1-methyl-pseudouridine (N1mPsU), or combinations thereof.

In some embodiments, mRNAs of the present disclosure (e.g., mRNAs encoding one or more CARs described herein) may contain modifications of the bases of the nucleotides (base modifications). A modified nucleotide which contains a base modification is also called a base-modified nucleotide.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs of the present disclosure (e.g., mRNAs encoding one or more CARs described herein) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′ triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. In some embodiments, a cap comprises a Cap0 structure. A cap0 structures lack a 2′-O-methyl residue of the ribose attached to bases 1 and 2. In some embodiments, a cap comprises an AGCap1 structure. An AGCap1 structures has a 2′-O-methyl residue at base 2. In some embodiments, a cap comprises a Cap2 structure. Cap2 structures have a 2′-O-methyl residue attached to both bases 2 and 3. In some embodiments, a cap structure comprises AGCap1, m6AGCap1, or Anti-Reverse Cap Analog (ARCA). In some embodiments, a modified mRNA of the present disclosure comprises an m6AGCap1 and modified nucleotides comprising pseudouridine (PsU).

In some embodiments, mRNAs of the present disclosure (e.g., mRNAs encoding one or more CARs described herein) include a 3′ poly(A) tail structure. A poly(A) tail on the 3′ terminus of mRNA typically includes about 10 to 400 adenosine nucleotides (SEQ ID NO: 73) (e.g., about 100 to 400 adenosine nucleotides, about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly(C) tail on the 3′ terminus of mRNA typically include about 10 to 200 cytosine nucleotides (SEQ ID NO: 74) (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). A poly(C) tail may be added to a poly(A) tail or may be a substitute for the poly(A) tail.

In some embodiments, mRNAs of the present disclosure (e.g., mRNAs encoding one or more CARs described herein) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

### Administration of Additional Payloads

In some embodiments, methods of the present disclosure comprise one or more steps of treating an immune cell (e.g., a stem cell, macrophage, monocyte, or dendritic cell) during the process of modifying the immune cell. In some embodiments, methods of the present disclosure comprise one or more steps of administering to a subject an additional payload for modulating an immune cell (e.g., a stem cell, macrophage, monocyte, or dendritic cell) during the process of modifying the immune cell (e.g., with a payload comprising a CAR). In some embodiments, a composition may comprise one or more additional payloads. In some embodiments, a composition may comprise one or more additional payloads in the same delivery vehicle as one or more nucleic acid molecules. In some embodiments, a composition may comprise one or more additional payloads in a different delivery vehicle than the one used with one or more nucleic acid molecules.

In some embodiments, methods of the present disclosure comprise a step of treating an immune cell (e.g., a stem cell, macrophage, monocyte, or dendritic cell) with a modulator of a pathway activated by in vitro transcribed mRNA. In some embodiments, an additional payload may be or comprise a modulator of a pathway activated by in vitro transcribed mRNA. In vitro transcribed (IVT) mRNA is recognized by various endosomal innate immune receptors (Toll-like receptor 3 (TLR3), TLR7 and TLR8) and cytoplasmic innate immune receptors (protein kinase RNA-activated (PKR), retinoic acid-inducible gene I protein (RIG-I), melanoma differentiation-associated protein 5 (MDAS) and 2′-5′-oligoadenylate synthase (OAS)). Signaling through these different pathways results in inflammation associated with type 1 interferon (IFN), tumor necrosis factor (TNF), interleukin-6 (IL-6), IL-12 and the activation of cascades of transcriptional programs. Overall, these create a pro-inflammatory microenvironment poised for inducing specific immune responses. Moreover, downstream effects such as slow-down of translation by eukaryotic translation initiation factor 2α (eIF2α) phosphorylation, enhanced RNA degradation by ribonuclease L (RNaseL), and overexpression and inhibition of replication of self-amplifying mRNA are of relevance for the pharmacokinetics and pharmacodynamics of IVT mRNA.

In some embodiments, a modulator of a pathway activated by in vitro transcribed mRNA comprises an RNase inhibitor. In some embodiments, a modulator of a pathway activated by in vitro transcribed mRNA comprises an RNaseL, RNase T2 or RNase1 inhibitor. In some embodiments, a modulator of a pathway activated by in vitro transcribed mRNA comprises an RNaseL inhibitor. In some embodiments, an RNaseL inhibitor comprises sunitinib. In some embodiments, an RNaseL inhibitor comprises ABCE1.

In some embodiments, treating an immune cell (e.g., a stem cell, macrophage, monocyte, or dendritic cell) with an RNaseL inhibitor increases mRNA stability in a modified immune cell relative to mRNA stability in a modified immune cell of the same type that was not treated with an RNaseL inhibitor. In some embodiments, treating an immune cell (e.g., a stem cell, macrophage, monocyte, or dendritic cell) with an RNaseL inhibitor increases CAR expression in a modified immune cell relative to CAR expression in a modified immune cell of the same type that was not treated with an RNaseL inhibitor. In some embodiments, treating an immune cell (e.g., a stem cell, macrophage, monocyte, or dendritic cell) with an RNaseL inhibitor increases effector activity in a modified immune cell relative to effector activity in a modified immune cell of the same type that was not treated with an RNaseL inhibitor.

In some embodiments, administering to a subject an RNaseL inhibitor increases mRNA stability in a modified immune cell (e.g., a stem cell, macrophage, monocyte, or dendritic cell) relative to mRNA stability in a modified immune cell of the same type in a subject that that was not administered an RNaseL inhibitor. In some embodiments, administering to a subject an RNaseL inhibitor increases CAR expression in a modified immune cell (e.g., a stem cell, macrophage, monocyte, or dendritic cell) relative to CAR expression in a modified immune cell of the same type in a subject that was not administered an RNaseL inhibitor. In some embodiments, administering to a subject an RNaseL inhibitor increases effector activity in a modified immune cell (e.g., a stem cell, macrophage, monocyte, or dendritic cell) relative to effector activity in a modified immune cell of the same type in a subject that was not administered an RNaseL inhibitor.

In some embodiments of the present disclosure, a step of treating an immune cell (e.g., a stem cell, macrophage, monocyte, or dendritic cell) occurs before a step of delivering an mRNA to the immune cell. In some embodiments of the present disclosure, a step of administering an additional payload to a subject occurs before a step of administering a composition comprising an mRNA to the subject.

In some embodiments, methods of the present disclosure comprise a step of culturing an immune cell (e.g., a stem cell, macrophage, monocyte, or dendritic cell) with a cytokine or immune stimulating recombinant protein. In some embodiments, methods of the present disclosure comprise a step of administering to a subject a cytokine or immune stimulating recombinant protein. In some embodiments, a cytokine comprises IFN-α, IFN—P, IFN-γ, TNFα, IL-6, STNGL, LPS, a CD40 agonist, a 4-1BB ligand, recombinant 4-1BB, a CD19 agonist, a TLR agonist (e.g., TLR-1, TLR-2, TLR-3, TLR-4, TLR-5, TLR-6, TLR-7, TLR-8 or TLR-9), TGF-β (e.g., TGF-β1, TGF-β2, or TGF-β3), a glucocorticoid, an immune complex, interleukin-1 alpha (IL-1α), IL-1β, IL-2, IL-3, IL-4, IL-5, IL-6, IL-7, IL-9, IL-10, IL-12, IL-13, IL-14, IL-15, IL-16, IL-17, IL-18, IL-20, granulocyte-macrophage colony-stimulating factor (GM-CSF), granulocyte colony-stimulating factor (G-CSF), Leukemia inhibitory factor (LIF), oncostatin M (OSM), TNF-β, CD154, lymphotoxin beta (LT-β), an A proliferation-inducing ligand (APRIL), CD70, CD153, glucocorticoid-induced TNF receptor ligand (GITRL), tumor necrosis factor superfamily member 14 (TNFSF14), OX40L (CD252), TALL-1 (Tumor necrosis factor ligand superfamily member 13B—TNFSF13B), TNF-related apoptosis-inducing ligand (TRAIL), TNF-related weak inducer of apoptosis (TWEAK), TNF-related activation-induced cytokine (TRANCE), erythropoietin (Epo), thyroid peroxidase precursor (Tpo), FMS-related tyrosine kinase 3 ligand (FLT-3L), stem cell factor (SCF), macrophage colony-stimulating factor (M-CSF), merozoite surface protein (MSP), a Nucleotide-binding oligomerization domain-containing protein (NOD) ligand (e.g., NOD1, NOD2, or NOD1/2 agonists), a RIG-I-like receptor (RLR) ligand (e.g., 5′ppp-dsRNA, 3p-hpRNA, Poly(I:C), or Poly(dA:dT)), a C-type lectin receptor (CLR) ligand (e.g., curdlan, β-glucan, HKCA, laminarin, pustulan, scleroglucan, WGP dispersible, WGP soluble, zymosan, zymosan depleted, furfurman, b-GlcCer, GlcC14C18, HKMT, TDB, TDB-HS15, or TDM), a cyclic dinucleotide sensor ligand (e.g., C-Gas agonist or stimulator of interferon gene (STING) ligand), an inflammasome inducer (e.g., alum, ATP, CPPD crystals, hemozoin, MSU crystals, Nano-SiO2, Nigericin, or TDB), an aryl hydrocarbon (AhR) ligand (e.g., FICZ, indirubin, ITE, or L-kynurenine), an alpha-protein kinase 1 (ALPK1) ligand, a multi-PRR ligand, an NFKB/NFAT activator (e.g., concavalin A, ionomycin, PHA-P, or PMA) or combinations thereof. In some embodiments, a cytokine comprises IFN-β.

In some embodiments of the present disclosure, a step of culturing an immune cell (e.g., a stem cell, macrophage, monocyte, or dendritic cell) occurs after a step of delivering an mRNA to the immune cell. In some embodiments of the present disclosure, a step of administering to a subject a cytokine or immune stimulating recombinant protein occurs after a step of administering a composition comprising an mRNA to the subject.

In some embodiments, culturing a modified immune cell (e.g., a stem cell, macrophage, monocyte, or dendritic cell) with a cytokine or immune stimulating recombinant protein increases the viability of the modified immune cell relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases protein (e.g., at least one CAR described herein) expression in the modified immune cell relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases longevity of protein (e.g., at least one CAR described herein) expression relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases effector activity of the modified immune cell relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases pro-inflammatory (M1) polarization of the modified immune cell relative to a modified immune cell of the same type that was not cultured with the cytokine or immune stimulating recombinant protein.

In some embodiments, administering to a subject a cytokine or immune stimulating recombinant protein increases the viability of a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases protein (e.g., at least one CAR described herein) expression of a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases longevity of protein (e.g., at least one CAR described herein) expression in a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases effector activity of a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases pro-inflammatory (M1) polarization of a modified immune cell (e.g., a stem cell, macrophage, monocyte, or dendritic cell) in the subject relative to a modified immune cell of the same type in a subject that was not administered the cytokine or immune stimulating recombinant protein.

### Methods of Altering the Inflammatory Phenotype of a Population of Cells

In some embodiments, methods of the present disclosure comprise altering the inflammatory phenotype of a population of cells. In some embodiments, methods of altering the inflammatory phenotype of a population of cells comprises contacting the population of cells with a modified immune cell (e.g., a stem cell, macrophage, monocyte, or dendritic cell) as described herein. In some embodiments, a population of cells comprises macrophages, monocytes, dendritic cells, T cells, NK cells, or combinations thereof. In some embodiments, a modified immune cell (e.g., a stem cell, macrophage, monocyte, or dendritic cell) as described herein secretes one or more inhibitory RNAs, which alters the inflammatory phenotype of a population of cells (e.g., bystander cells). In some embodiments, one or more inhibitory RNAs are packaged within an extracellular vehicle. In some embodiments, one or more inhibitory RNAs are packaged within an exosome.

In some embodiments, the inflammatory phenotype of the population of cells is altered from anti-inflammatory to non-activated. In some embodiments, the inflammatory phenotype of the population of cells is altered from pro-inflammatory to non-activated. In some embodiments, the inflammatory phenotype of the population of cells is altered from anti-inflammatory to pro-inflammatory. In some embodiments, the inflammatory phenotype of the population of cells is altered from pro-inflammatory to anti-inflammatory.

### Modified Immune Cells

In some embodiments, a modified immune cell (e.g., a stem cell, macrophage, monocyte, or dendritic cell) is made by methods of the present disclosure. In some embodiments, a modified immune cell comprises at least one CAR described herein. In some embodiments a modified immune cell comprises one or more nucleic acids encoding at least one CAR described herein. In some embodiments, at least one CAR described herein comprises at least one extracellular domain, at least one transmembrane domain and at least one intracellular domain. In some embodiments, a modified immune cell of the present disclosure comprises one or more nucleic acids constructs comprising a promoter, a gene of interest, a 3′ untranslated region (UTR), and one or more introns, wherein the one or more introns comprise one or more inhibitory nucleic acids, wherein the one or more inhibitory nucleic acids encode one or more inhibitory RNAs, and, wherein the gene of interest encodes a chimeric antigen receptor (CAR). In some embodiments, a modified immune cell of the present disclosure comprises a CAR and one or more inhibitory RNAs.

In some embodiments, a modified immune cell (e.g., a stem cell, macrophage, monocyte, or dendritic cell) of the present disclosure comprises at least one CAR described herein comprising an extracellular domain described herein that binds a tumor antigen, such as an antigen that is specific for a tumor or cancer of interest. In some embodiments, a tumor antigen comprises one or more antigenic cancer epitopes. In some embodiments, a tumor antigen comprises mesothelin.

In some embodiments, a modified immune cell (e.g., a stem cell, macrophage, monocyte, or dendritic cell) comprising a modified mRNA encoding at least one CAR provided herein exhibits increased viability relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a modified immune cell (e.g., a stem cell, macrophage, monocyte, or dendritic cell) comprising a modified mRNA encoding at least one CAR described herein exhibits increased expression of an mRNA encoding at least one CAR described herein relative to a modified immune cell of the same type comprising unmodified mRNA encoding a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a modified immune cell (e.g., a stem cell, macrophage, monocyte, or dendritic cell) comprising at least one CAR as provided herein exhibits increased CAR expression relative to a modified immune cell of the same type comprising unmodified mRNA encoding a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a modified immune cell (e.g., a stem cell, macrophage, monocyte, or dendritic cell) comprising a modified mRNA encoding at least one CAR as provided herein exhibits increased longevity of a mRNA encoding at least one CAR relative to a modified immune cell of the same type comprising unmodified mRNA encoding a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a modified immune cell (e.g., a stem cell, macrophage, monocyte, or dendritic cell) comprising a modified mRNA encoding a CAR as provided herein exhibits increased longevity of the CAR relative to a modified immune cell of the same type comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a modified immune cell (e.g., a stem cell, macrophage, monocyte, or dendritic cell) comprising a CAR as provided herein exhibits increased effector activity relative to a modified immune cell of the same type comprising unmodified mRNA encoding a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR). In some embodiments, a modified immune cell (e.g., a stem cell, macrophage, monocyte, or dendritic cell) comprising a modified mRNA encoding a CAR as provided herein exhibits increased pro-inflammatory (M1) polarization relative to a modified immune cell of the same type comprising unmodified mRNA encoding a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a modified immune cell (e.g., a stem cell, macrophage, monocyte, or dendritic cell) comprising a CAR as provided herein maintains a pro-inflammatory phenotype over time. In some embodiments, a modified immune cell (e.g., a stem cell, macrophage, monocyte, or dendritic cell) comprising a CAR as provided herein maintains a pro-inflammatory phenotype at least 4 hours, 2 days, 4 days, 7 days, 14 days, 28 days, and/or 40 days after an immune cell is modified with a nucleic acid encoding the CAR.

In some embodiments, a modified immune cell (e.g., a stem cell, macrophage, monocyte, or dendritic cell) comprising a CAR as provided herein maintains an anti-inflammatory phenotype over time. In some embodiments, a modified immune cell (e.g., a stem cell, macrophage, monocyte, or dendritic cell) comprising a CAR as provided herein maintains an anti-inflammatory phenotype at least 4 hours, 2 days, 4 days, 7 days, 14 days, 28 days and/or 40 days after an immune cell is modified with a nucleic acid encoding the CAR.

In some embodiments, a modified immune cell (e.g., a stem cell, macrophage, monocyte, or dendritic cell) comprising a CAR as provided herein maintains a pro-inflammatory phenotype and/or otherwise resists subversion when challenged by anti-inflammatory cytokines. In some embodiments, the sensitivity of a modified immune cell to environmental cytokines is measured by generating a dose-response curve of pro-inflammatory markers by treating modified immune cells comprising a CAR as provided herein. In some embodiments, the sensitivity of a modified immune cell to environmental cytokines is measured by generating a dose-response curve of pro-inflammatory markers by treating modified immune cells comprising a CAR as provided herein.

In some embodiments, a modified immune cell (e.g., a stem cell, macrophage, monocyte, or dendritic cell) comprising a CAR as provided herein maintains an anti-inflammatory phenotype and/or otherwise resists subversion when challenged by pro-inflammatory cytokines. In some embodiments, the sensitivity of a modified immune cell to environmental cytokines is measured by generating a dose-response curve of anti-inflammatory markers by treating modified immune cells comprising a CAR as provided herein with increasing concentrations of pro-inflammatory cytokines. In some embodiments, the sensitivity of a modified immune cell to environmental cytokines is measured by generating a dose-response curve of anti-inflammatory markers by treating modified immune cells comprising a CAR as provided herein with increasing concentrations of anti-inflammatory cytokines.

In some embodiments, a modified immune cell (e.g., a stem cell, macrophage, monocyte, or dendritic cell) comprising a CAR as provided herein has minimal effects on neighboring cells. In some embodiments, a modified immune cell (e.g., a stem cell, macrophage, monocyte, or dendritic cell) comprising a CAR as provided herein has significant effects on neighboring cells. In some embodiments, the effect of a modified immune cell (e.g., a stem cell, macrophage, monocyte, or dendritic cell) comprising a CAR as provided herein can be tested by co-culturing modified immune cells with unmodified immune cells and using flow cytometry to analyze the expression of pro-inflammatory and anti-inflammatory markers in the unmodified cells. In some embodiments, modified immune cells and unmodified immune cells can be co-cultured in a culture dish where the modified immune cells and unmodified immune cells contact each other. In some embodiments, modified immune cells and unmodified immune cells can be co-cultured in a culture dish where the modified immune cells and unmodified immune cells are separated by a transwell assay membrane.

In some embodiments, a modified immune cell (e.g., a stem cell, macrophage, monocyte, or dendritic cell) comprising a CAR as provided herein has minimal cytotoxic effects on neighboring cells. In some embodiments, a modified immune cell (e.g., a stem cell, macrophage, monocyte, or dendritic cell) comprising a CAR as provided herein has significant cytotoxic effects on neighboring cells (e.g., cancer cells). In some embodiments, modifying an immune cell to comprise a CAR as provided herein is not cytotoxic to the modified immune cell. In some embodiments, RNAseq data from modified immune cells are examined to determine if upregulation of genes indicative of cytotoxic effects is present.

In some embodiments, expression of a CAR provided herein in a modified immune cell (e.g., a stem cell, macrophage, monocyte, or dendritic cell) increases at least one targeted effector function (e.g., phagocytosis, targeted cellular cytotoxicity, antigen presentation, or cytokine secretion) of the modified immune cell relative to an unmodified immune cell or a modified cell comprising a similar CAR (e.g., a CAR comprising a different anti-mesothelin antigen binding domain and/or without one or both of (i) a CD8 or CD28 extracellular hinge domain, and (ii) a CD8 or CD28 transmembrane domain, but with the other components of the comparator CAR).

In some embodiments, a modified immune cell (e.g., a stem cell, macrophage, monocyte, or dendritic cell) comprising a CAR as provided herein comprises one or more control systems including, but not limited to: a safety switch (e.g., an on switch, an off switch, a suicide switch), transcriptional control (e.g. cell-specific promoters, cell-state specific promoters, promoters downstream of CAR activation, promoters downstream of endogenous signaling pathways, or drug-inducible transcription), post-transcriptional control of CAR mRNA (e.g. RNA-based inhibition with endogenous or recombinant miRNA), or post-translational control of CAR structure or stability (e.g. a CAR whose intracellular domain conditionally associates with the full structure by drug/light-inducible association (to allow signaling) or dissociation (to inhibit signaling), or whose stability is drug-regulated for inducible stabilization (to allow signaling) or degradation (to inhibit signaling)). These control systems can be combined to create logic gates, for example an AND gate (e.g. a CAR with a CAR-inducible promoter and cytosolic domain that associates in a drug-dependent manner, thus requiring CAR activation and the presence of a small molecule), an OR gate (e.g. a CAR under control of a promoter that is transcriptionally active following CAR activation or small molecule addition), and/or a NOT gate (e.g. a CAR whose mRNA is degraded by endogenous miRNA expressed in natural immune cell signaling states (such as miRNA upregulated by a particular cytokine signaling pathway, thus only expressing a CAR in the absence of this cytokine)).

In some embodiments, a modified immune cell (e.g., a stem cell, macrophage, monocyte, or dendritic cell) of the present disclosure comprises one or more inhibitory RNAs selected from the group consisting of antisense RNA (asRNA), cis-natural antisense transcript (cis-NAT), CRISPR RNA (crRNA), guide RNA (gRNA), long noncoding RNA (lncRNA), microRNA (miRNA), piwi-interacting RNA (piRNA), small interfering RNA (siRNA), short hairpin RNA (shRNA), trans-acting siRNA (tasiRNA), and repeat associated siRNA (rasiRNA).

In some embodiments, a modified immune cell (e.g., a stem cell, macrophage, monocyte, or dendritic cell) of the present disclosure comprises one or more shRNA. In some embodiments, a modified immune cell (e.g., a stem cell, macrophage, monocyte, or dendritic cell) of the present disclosure comprises one or more shRNA comprising an miRNA scaffold. In some embodiments, an miRNA scaffold comprises an miRNA-155 5′ scaffold, an miRNA-155 3′ scaffold, an miRNA-30 5′ scaffold, an miRNA-30 3′ scaffold, an miRNA-16 5′ scaffold, an miRNA-16 3′ scaffold, an miRNA-125 5′ scaffold, an miRNA-125 3′ scaffold, an miRNA-223 5′ scaffold, or an miRNA-223 3′ scaffold.

In some embodiments, a modified immune cell (e.g., a stem cell, macrophage, monocyte, or dendritic cell) of the present disclosure comprises one or more shRNA comprising a guide strand. In some embodiments, a guide strand is about 19-24 bases in length. In some embodiments, a guide strand has a G/C content of about 36%-50%. In some embodiments, a guide strand comprises a nucleic acid sequence that is reverse complementary to a target gene transcript comprising a target nucleic acid sequence.

In some embodiments, a modified immune cell (e.g., a stem cell, macrophage, monocyte, or dendritic cell) of the present disclosure comprises one or more shRNA comprising a passenger strand. In some embodiments, a passenger strand is 1-2 bases shorter than a corresponding guide strand. In some embodiments, a passenger strand is not fully complementary to the guide strand.

In some embodiments, a modified immune cell (e.g., a stem cell, macrophage, monocyte, or dendritic cell) of the present disclosure comprises one or more shRNA comprising a loop.

In some embodiments, a modified immune cell (e.g., a stem cell, macrophage, monocyte, or dendritic cell) of the present disclosure comprises a target gene transcript encoding human ATG7, C/EBP-alpha, C/EBP-beta, CD36, CLEC1A, FATS, GOLM1, HAVCR2, ITGAD, KLF4, KLF6, LILRB2, LILRB4, MAF, MafB, PD-LI, PIK3CG, PIK3CG, PPARα, PPARγ, PTGS2, SIGLEC10, SIRPα, SLC15A3, STAT3, STAT6, TNFRSF1B, TOX, TREM2, YTHDF2, or ZFP36. In some embodiments, a modified immune cell (e.g., a stem cell, macrophage, monocyte, or dendritic cell) of the present disclosure comprises a target gene transcript encoding human SIRPα. In some embodiments, a modified immune cell (e.g., a stem cell, macrophage, monocyte, or dendritic cell) of the present disclosure comprises a target gene transcript encoding human PD-L1.

In some embodiments, a modified immune cell (e.g., a stem cell, macrophage, monocyte, or dendritic cell) comprising a CAR as described herein maintains a pro-inflammatory phenotype over time. In some embodiments, a modified immune cell (e.g., a stem cell, macrophage, monocyte, or dendritic cell) comprising a CAR as described herein maintains a pro-inflammatory phenotype at least 4 hours, 2 days, 4 days, 7 days, 14 days, and/or 28 days after an immune cell is modified with a nucleic acid encoding the CAR.

In some embodiments, a modified immune cell (e.g., a stem cell, macrophage, monocyte, or dendritic cell) comprising a CAR as described herein maintains an anti-inflammatory phenotype over time. In some embodiments, a modified immune cell (e.g., a stem cell, macrophage, monocyte, or dendritic cell) comprising a CAR as described herein maintains an anti-inflammatory phenotype at least 4 hours, 2 days, 4 days, 7 days, 14 days, and/or 28 days after an immune cell is modified with a nucleic acid encoding the CAR.

In some embodiments, a modified immune cell (e.g., a stem cell, macrophage, monocyte, or dendritic cell) comprising a CAR as described herein maintains a pro-inflammatory phenotype and/or otherwise resists subversion when challenged by anti-inflammatory cytokines. In some embodiments, the sensitivity of a modified immune cell to environmental cytokines is measured by generating a dose-response curve of pro-inflammatory markers by treating modified immune cells comprising a CAR as described herein with increasing concentrations of anti-inflammatory cytokines. In some embodiments, the sensitivity of a modified immune cell to environmental cytokines is measured by generating a dose-response curve of pro-inflammatory markers by treating modified immune cells comprising a CAR as described herein with increasing concentrations of pro-inflammatory cytokines.

In some embodiments, a modified immune cell (e.g., a stem cell, macrophage, monocyte, or dendritic cell) comprising a CAR as described herein maintains an anti-inflammatory phenotype and/or otherwise resists subversion when challenged by pro-inflammatory cytokines. In some embodiments, the sensitivity of a modified immune cell to environmental cytokines is measured by generating a dose-response curve of anti-inflammatory markers by treating modified immune cells comprising a CAR as described herein with increasing concentrations of pro-inflammatory cytokines. In some embodiments, the sensitivity of a modified immune cell to environmental cytokines is measured by generating a dose-response curve of anti-inflammatory markers by treating modified immune cells comprising a CAR as described herein with increasing concentrations of anti-inflammatory cytokines.

In some embodiments, a modified immune cell (e.g., a stem cell, macrophage, monocyte, or dendritic cell) comprising a CAR as described herein has minimal effects on neighboring cells. In some embodiments, a modified immune cell (e.g., a stem cell, macrophage, monocyte, or dendritic cell) comprising a CAR as described herein has significant effects on neighboring cells. In some embodiments, the effect of a modified immune cell (e.g., a stem cell, macrophage, monocyte, or dendritic cell) comprising a CAR as described herein on an unmodified cell (e.g., an immune cell that doesn't comprise a CAR as described herein) can be tested by co-culturing modified immune cells with unmodified immune cells and using flow cytometry to analyze the expression of pro-inflammatory and anti-inflammatory markers in the unmodified cells. In some embodiments, modified immune cells and unmodified immune cells can be co-cultured in a culture dish where the modified immune cells and unmodified immune cells contact each other. In some embodiments, modified immune cells and unmodified immune cells can be co-cultured in a culture dish where the modified immune cells and unmodified immune cells are separated by a transwell assay membrane.

In some embodiments, a modified immune cell (e.g., a stem cell, macrophage, monocyte, or dendritic cell) comprising a CAR as described herein has minimal cytotoxic effects on neighboring cells. In some embodiments, a modified immune cell (e.g., a stem cell, macrophage, monocyte, or dendritic cell) comprising a CAR as described herein has significant cytotoxic effects on neighboring cells (e.g., cancer cells). In some embodiments, modifying an immune cell to comprise a CAR as described herein is not cytotoxic to the modified immune cell. In some embodiments, RNAseq data from modified immune cells are examined to determine if upregulation of genes indicative of cytotoxic effects is present.

In some embodiments, a modified immune cell (e.g., a stem cell, macrophage, monocyte, or dendritic cell) comprising a CAR as described herein may comprise one or more control systems including, but not limited to: a safety switch (e.g., an on switch, an off switch, a suicide switch), transcriptional control (e.g. cell-specific promoters, cell-state specific promoters, promoters downstream of CAR activation, promoters downstream of endogenous signaling pathways, or drug-inducible transcription), post-transcriptional control of CAR mRNA (e.g. RNA-based inhibition with endogenous or recombinant miRNA), or post-translational control of a CAR's structure or stability (e.g. a CAR whose intracellular domain conditionally associates with the full structure by drug/light-inducible association (to allow signaling) or dissociation (to inhibit signaling), or whose stability is drug-regulated for inducible stabilization (to allow signaling) or degradation (to inhibit signaling)). These control systems can be combined to create logic gates, for example an AND gate (e.g. a CAR with a CAR-inducible promoter and cytosolic domain that associates in a drug-dependent manner, thus requiring CAR activation and the presence of a small molecule), an OR gate (e.g. a CAR under control of a promoter that is transcriptionally active following CAR activation or small molecule addition), and/or a NOT gate (e.g. a CAR whose mRNA is degraded by endogenous miRNAs expressed in natural immune cell signaling states (such as miRNAs upregulated by a particular cytokine signaling pathway, thus only expressing CAR in the absence of this cytokine)).

In some embodiments, an effector cell (i.e., a modified immune cell of the present disclosure (e.g., a stem cell, macrophage, monocyte, or dendritic cell) comprising a nucleic acid construct as described herein secretes one or more inhibitory RNAs of the present disclosure. In some embodiments, one or more inhibitory RNAs are packaged within an extracellular vesicle. In some embodiments, one or more inhibitory RNAs are packaged within an exosome. In some embodiments, secreted inhibitory RNA affects the phenotype of bystander cells. In some embodiments, bystander cells are bystander macrophages, bystander monocytes, bystander dendritic cells, or bystander stem cells. In some embodiments, bystander cells are skewed to an anti-tumor phenotype. In some embodiments, bystander cells are skewed to an anti-inflammatory phenotype. In some embodiments, bystander macrophages are skewed to an M1 phenotype. In some embodiments, bystander macrophages are skewed to an M2 phenotype.

### Assays

A variety of assays may be performed to confirm the presence of a nucleic acid construct described herein and/or the presence of a protein (e.g., a CAR) in an immune cell (e.g., a stem cell, macrophage, monocyte, or dendritic cell). For example, such assays include molecular biological assays well known to those of skill in the art, such as Southern and Northern blotting, RT-PCR, and PCR; and biochemical assays, such as detecting the presence or absence of a particular peptide, e.g., by immunological means (ELISAs and Western blots). Other assays of the present disclosure include, for example, fluorescence-activated cell sorting (FACS), immunofluorescent microscopy, MSD cytokine analysis, mass spectrometry (MS), RNA-Seq and functional assays.

A variety of assays may be performed to determine various characteristics of a modified immune cell (e.g., a stem cell, macrophage, monocyte, or dendritic cell), such as, but not limited to, immune cell viability, nucleic acid expression, nucleic acid longevity, protein (e.g., CAR) expression, protein (e.g., CAR) longevity, effector activity, and pro-inflammatory (M1) polarization. For example, such assays include flow cytometry, quantitative PCR, and in vitro functional assays such as cytokine/chemokine secretion, phagocytosis, and specific lysis assays of target tumor cells.

### Nucleic Acid Constructs

The present disclosure provides, among other things, nucleic acid molecules encoding at least one CAR described herein or a fragment thereof. An immune cell (e.g., stem cell, macrophage, monocyte, or dendritic cell) can comprise a nucleic acid molecule (e.g., an exogenous nucleic acid molecule) encoding at least one polypeptides (e.g., one or more CARs of the present disclosure) described herein.

Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some version contain an intron(s). The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “operably linked” or “transcriptional control” refers to functional linkage between a regulatory sequence and a heterologous nucleic acid sequence resulting in expression of the heterologous nucleic acid sequence. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Nucleic acid molecules encoding at least one protein (e.g., a CAR of the present disclosure) described herein or a fragment thereof can be a DNA molecule, an RNA molecule, or a combination thereof. In some embodiments, a nucleic acid molecule comprises or is a messenger RNA (mRNA) transcript encoding at least one protein (e.g., a CAR of the present disclosure) described herein or a fragment thereof. In some embodiments, a nucleic acid molecule comprises or is a DNA construct encoding at least one protein (e.g., a CAR of the present disclosure) described herein or a fragment thereof.

In some embodiments, all or a fragment of a protein (e.g., at least one CAR of the present disclosure) described herein is encoded by a codon optimized nucleic acid molecule, e.g., for expression in a cell (e.g., a mammalian cell). A variety of codon optimization methods are known in the art, e.g., as disclosed in U.S. Pat. Nos. 5,786,464 and 6,114,148, each of which is hereby incorporated by reference in its entirety.

Expression of nucleic acids described herein may be achieved by operably linking a nucleic acid encoding a protein (e.g., at least one CAR of the present disclosure) or fragment thereof to a promoter in an expression vector. Exemplary promoters (e.g., constitutive promoters) include, but are not limited to, an elongation factor-1α promoter (EF-1a) promoter, immediate early cytomegalovirus (CMV) promoter, ubiquitin C promoter, phosphoglycerokinase (PGK) promoter, simian virus 40 (SV40) early promoter, mouse mammary tumor virus (MMTV) promoter, human immunodeficiency virus (HIV) long terminal repeat (LTR) promoter, Moloney murine leukemia virus (MoMuLV) promoter, an avian leukemia virus promoter, an Epstein-Barr virus immediate early promoter, a Rous sarcoma virus promoter, an actin promoter, a myosin promoter, a hemoglobin promoter, or a creatine kinase promoter. Examples of inducible promoters include, but are not limited to a metallothionine promoter, a glucocorticoid promoter, a progesterone promoter, and a tetracycline promoter. A vector can also comprise additional promoter elements, e.g., enhancers, to regulate the frequency of transcriptional initiation.

In some embodiments, a vector comprising a nucleic acid molecule encoding a protein (e.g., at least one CAR of the present disclosure) or fragment thereof comprises or is a viral vector. Viral vector technology is well known in the art and is described (e.g., in Sambrook et al., 2012, MOLECULAR CLONING: A LABORATORY MANUAL, volumes 1-4, Cold Spring Harbor Press, NY). Examples of viral vectors include, but are not limited to, adenoviral vectors, adeno-associated viral vectors, or retroviral vectors (e.g., a lentiviral vector or a gammaretroviral vector). In some embodiments, a vector comprises a lentiviral vector (e.g., as described in U.S. Pat. No. 9,149,519 or International Publication No. WO 2017/044487, each of which is hereby incorporated by reference in its entirety).

In some embodiments, a viral vector comprises an adenoviral vector. Adenoviruses are a large family of viruses containing double stranded DNA. They replicate within the nucleus of a host cell, using the host's cell machinery to synthesize viral RNA, DNA and proteins. Adenoviruses are known in the art to affect both replicating and non-replicating cells, to accommodate large transgenes, and to code for proteins without integrating into the host cell genome. In some embodiments, an adenoviral vector comprises an Ad2 vector or an Ad5 vector (e.g., Ad5f35 adenoviral vector, e.g., a helper-dependent Ad5F35 adenoviral vector).

In some embodiments, a viral vector is an adeno-associated virus (AAV) vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012)). Methods for generating and using recombinant AAV (rAAV) vectors are described, for example, in U.S. Pat. Nos. 5,139,941 and 4,797,368.

Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. Generally, any AAV serotype may be used to deliver a protein (e.g., at least one CAR of the present disclosure) or fragment thereof described herein. In some embodiments, an AAV serotype has a tropism for a particular tissue.

In some embodiments, CRISPR/Cas9 system has recently been shown to facilitate high levels of precise genome editing using adeno associated viral (AAV) vectors to serve as donor template DNA during homologous recombination (HR).

In some embodiments, a vector comprises a gammaretroviral vector (e.g., as described in Tobias Maetzig et al., “Gammaretroviral Vectors: Biology, Technology and Application” Viruses. 2011 June; 3(6): 677-713, which is hereby incorporated by reference in its entirety). Exemplary gammaretroviral vectors include Murine Leukemia Virus (MLV), Spleen-Focus Forming Virus (SFFV), and Myeloproliferative Sarcoma Virus (MPSV), and vectors derived therefrom.

In some embodiments, a vector comprises two or more nucleic acid sequences encoding a CAR, e.g., at least one CAR described herein, and a second CAR, e.g., a different CAR described herein. In some embodiments, two or more nucleic acid sequences encoding a CAR and a second CAR are encoded by a single nucleic molecule, e.g., in same frame and as a single polypeptide chain. In some embodiments, two or more CARs are separated by one or more cleavage peptide sites (e.g., an auto-cleavage site or a substrate for an intracellular protease). In certain embodiments, a cleavage peptide comprises a porcine teschovirus-1 (P2A) peptide, Thosea asigna virus (T2A) peptide, equine rhinitis A virus (E2A) peptide, foot-and-mouth disease virus (F2A) peptide, or a variant thereof.

In some embodiments, a vector comprises at least one nucleic acid sequence encoding a CAR, e.g., at least one CAR described herein, and at least one nucleic acid encoding at least one gene co-expressed with a CAR, e.g., a cytokine described herein (e.g., TNF, IL-12, IFN, GM-CSF, G-CSF, M-CSF, and/or IL-1) or a stimulatory ligand described herein (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and/or a B7-H3 ligand.

In some embodiments, a CAR of the present disclosure binds to mesothelin and is encoded by a nucleic acid sequence that is at least 80% identical to a sequence selected from Table 4. In some embodiments, a CAR of the present disclosure binds to mesothelin and is encoded by a nucleic acid sequence that is at least 85% identical to a sequence selected from Table 4. In some embodiments, a CAR of the present disclosure binds to mesothelin and is encoded by a nucleic acid sequence that is at least 90% identical to a sequence selected from Table 4. In some embodiments, a CAR of the present disclosure binds to mesothelin and is encoded by a nucleic acid sequence that is at least 95% identical to a sequence selected from Table 4. In some embodiments, a CAR of the present disclosure binds to mesothelin and is encoded by a nucleic acid sequence that is at least 96% identical to a sequence selected from Table 4. In some embodiments, a CAR of the present disclosure binds to mesothelin and is encoded by a nucleic acid sequence that is at least 97% identical to a sequence selected from Table 4. In some embodiments, a CAR of the present disclosure binds to mesothelin and is encoded by a nucleic acid sequence that is at least 98% identical to a sequence selected from Table 4. In some embodiments, a CAR of the present disclosure binds to mesothelin and is encoded by a nucleic acid sequence that is at least 99% identical to a sequence selected from Table 4. In some embodiments, a CAR of the present disclosure binds to mesothelin and is encoded by a nucleic acid sequence that is identical to a sequence selected from Table 4.

In some embodiments, a CAR of the present disclosure binds to mesothelin and is encoded by a nucleic acid sequence that differs from a sequence selected from Table 4 by no more than 100 substitutions, additions, or deletions. In some embodiments, a CAR of the present disclosure binds to mesothelin and is encoded by a nucleic acid sequence that differs from a sequence selected from Table 4 by no more than 75 substitutions, additions, or deletions. In some embodiments, a CAR of the present disclosure binds to mesothelin and is encoded by a nucleic acid sequence that differs from a sequence selected from Table 4 by no more than 50 substitutions, additions, or deletions. In some embodiments, a CAR of the present disclosure binds to mesothelin and is encoded by a nucleic acid sequence that differs from a sequence selected from Table 4 by no more than 40 substitutions, additions, or deletions. In some embodiments, a CAR of the present disclosure binds to mesothelin and is encoded by a nucleic acid sequence that differs from a sequence selected from Table 4 by no more than 30 substitutions, additions, or deletions. In some embodiments, a CAR of the present disclosure binds to mesothelin and is encoded by a nucleic acid sequence that differs from a sequence selected from Table 4 by no more than 20 substitutions, additions, or deletions. In some embodiments, a CAR of the present disclosure binds to mesothelin and is encoded by a nucleic acid sequence that differs from a sequence selected from Table 4 by no more than 10 substitutions, additions, or deletions. In some embodiments, a CAR of the present disclosure binds to mesothelin and is encoded by a nucleic acid sequence that differs from a sequence selected from Table 4 by no more than 9 substitutions, additions, or deletions. In some embodiments, a CAR of the present disclosure binds to mesothelin and is encoded by a nucleic acid sequence that differs from a sequence selected from Table 4 by no more than 8 substitutions, additions, or deletions. In some embodiments, a CAR of the present disclosure binds to mesothelin and is encoded by a nucleic acid sequence that differs from a sequence selected from Table 4 by no more than 7 substitutions, additions, or deletions. In some embodiments, a CAR of the present disclosure binds to mesothelin and is encoded by a nucleic acid sequence that differs from a sequence selected from Table 4 by no more than 6 substitutions, additions, or deletions. In some embodiments, a CAR of the present disclosure binds to mesothelin and is encoded by a nucleic acid sequence that differs from a sequence selected from Table 4 by no more than 5 substitutions, additions, or deletions. In some embodiments, a CAR of the present disclosure binds to mesothelin and is encoded by a nucleic acid sequence that differs from a sequence selected from Table 4 by no more than 4 substitutions, additions, or deletions. In some embodiments, a CAR of the present disclosure binds to mesothelin and is encoded by a nucleic acid sequence that differs from a sequence selected from Table 4 by no more than 3 substitutions, additions, or deletions. In some embodiments, a CAR of the present disclosure binds to mesothelin and is encoded by a nucleic acid sequence that differs from a sequence selected from Table 4 by no more than 2 substitutions, additions, or deletions. In some embodiments, a CAR of the present disclosure binds to mesothelin and is encoded by a nucleic acid sequence that differs from a sequence selected from Table 4 by no more than 1 substitution, addition, or deletion. In some embodiments, a CAR of the present disclosure binds to mesothelin and is encoded by a nucleic acid sequence that does not have any substitutions, additions, or deletions relative to a sequence selected from Table 4.

In some embodiments, a CAR antigen binding domain binds to mesothelin and is encoded by a nucleic acid sequence that is at least 80% identical to a sequence selected from Table 5. In some embodiments, a CAR antigen binding domain binds to mesothelin and is encoded by a nucleic acid sequence that is at least 85% identical to a sequence selected from Table 5. In some embodiments, a CAR antigen binding domain binds to mesothelin and is encoded by a nucleic acid sequence that is at least 90% identical to a sequence selected from Table 5. In some embodiments, a CAR antigen binding domain binds to mesothelin and is encoded by a nucleic acid sequence that is at least 95% identical to a sequence selected from Table 5. In some embodiments, a CAR antigen binding domain binds to mesothelin and is encoded by a nucleic acid sequence that is at least 96% identical to a sequence selected from Table 5. In some embodiments, a CAR antigen binding domain binds to mesothelin and is encoded by a nucleic acid sequence that is at least 97% identical to a sequence selected from Table 5. In some embodiments, a CAR antigen binding domain binds to mesothelin and is encoded by a nucleic acid sequence that is at least 98% identical to a sequence selected from Table 5. In some embodiments, a CAR antigen binding domain binds to mesothelin and is encoded by a nucleic acid sequence that is at least 99% identical to a sequence selected from Table 5. In some embodiments, a CAR antigen binding domain binds to mesothelin and is encoded by a nucleic acid sequence that is identical to a sequence selected from Table 5.

In some embodiments, a CAR antigen binding domain binds to mesothelin and is encoded by a nucleic acid sequence that differs from a sequence selected from Table 5 by no more than 50 substitutions, additions, or deletions. In some embodiments, a CAR antigen binding domain binds to mesothelin and is encoded by a nucleic acid sequence that differs from a sequence selected from Table 5 by no more than 40 substitutions, additions, or deletions. In some embodiments, a CAR antigen binding domain binds to mesothelin and is encoded by a nucleic acid sequence that differs from a sequence selected from Table 5 by no more than 30 substitutions, additions, or deletions. In some embodiments, a CAR antigen binding domain binds to mesothelin and is encoded by a nucleic acid sequence that differs from a sequence selected from Table 5 by no more than 20 substitutions, additions, or deletions. In some embodiments, a CAR antigen binding domain binds to mesothelin and is encoded by a nucleic acid sequence that differs from a sequence selected from Table 5 by no more than 10 substitutions, additions, or deletions. In some embodiments, a CAR antigen binding domain binds to mesothelin and is encoded by a nucleic acid sequence that differs from a sequence selected from Table 5 by no more than 9 substitutions, additions, or deletions. In some embodiments, a CAR antigen binding domain binds to mesothelin and is encoded by a nucleic acid sequence that differs from a sequence selected from Table 5 by no more than 8 substitutions, additions, or deletions. In some embodiments, a CAR antigen binding domain binds to mesothelin and is encoded by a nucleic acid sequence that differs from a sequence selected from Table 5 by no more than 7 substitutions, additions, or deletions. In some embodiments, a CAR antigen binding domain binds to mesothelin and is encoded by a nucleic acid sequence that differs from a sequence selected from Table 5 by no more than 6 substitutions, additions, or deletions. In some embodiments, a CAR antigen binding domain binds to mesothelin and is encoded by a nucleic acid sequence that differs from a sequence selected from Table 5 by no more than 5 substitutions, additions, or deletions. In some embodiments, a CAR antigen binding domain binds to mesothelin and is encoded by a nucleic acid sequence that differs from a sequence selected from Table 5 by no more than 4 substitutions, additions, or deletions. In some embodiments, a CAR antigen binding domain binds to mesothelin and is encoded by a nucleic acid sequence that differs from a sequence selected from Table 5 by no more than 3 substitutions, additions, or deletions. In some embodiments, a CAR antigen binding domain binds to mesothelin and is encoded by a nucleic acid sequence that differs from a sequence selected from Table 5 by no more than 2 substitutions, additions, or deletions. In some embodiments, a CAR antigen binding domain binds to mesothelin and is encoded by a nucleic acid sequence that differs from a sequence selected from Table 5 by no more than 1 substitution, addition, or deletion. In some embodiments, a CAR antigen binding domain binds to mesothelin and is encoded by a nucleic acid sequence that does not have any substitutions, additions, or deletions relative to a sequence selected from Table 5.

In some embodiments, the present disclosure provides nucleic acid molecules comprising a promoter, a gene of interest, a 3′ untranslated region (UTR), and one or more introns. In some embodiments, one or more introns of the present disclosure comprise one or more inhibitory nucleic acids of the present disclosure. In some embodiments, one or more inhibitory nucleic acids of the present disclosure encode one or more inhibitory RNAs of the present disclosure. In some embodiments, a gene of interest encodes at least one chimeric antigen receptor (CAR) described herein or a fragment thereof. A modified immune cell (e.g., stem cell, macrophage, monocyte, or dendritic cell) of the present disclosure can comprise a nucleic acid molecule (e.g., an exogenous nucleic acid molecule) comprising one or more introns of the present disclosure and encoding at least one protein (e.g., a CAR of the present disclosure) described herein. In some embodiments, the present disclosure provides nucleic acid molecules wherein expression of a CAR is increased relative to a reference nucleic acid molecule that lacks one or more introns.

### Introns

In some embodiments, one or more introns of the present disclosure comprise one, two, or three introns. In some embodiments, one or more introns of the present disclosure are located downstream of a promoter. In some embodiments, one or more introns of the present disclosure are located within a gene of interest. In some embodiments, one or more introns of the present disclosure are located within a 3′ UTR. In some embodiments, one or more introns of the present disclosure are located within a gene of interest and within a 3′ UTR. In some embodiments, one or more introns of the present disclosure comprise three inhibitory nucleic acids. In some embodiments, three inhibitory nucleic acids encode three inhibitory RNAs. In some embodiments, three inhibitory RNAs comprise the following miRNA scaffolds in order from 5′ to 3′: (a) miRNA 30, miRNA 30, miRNA 30, (b) miRNA 30, miRNA 155, miRNA 30, (c) miRNA 155, miRNA 30, miRNA 155, or (d) miRNA 155, miRNA 155, miRNA 155.

### Inhibitory Nucleic Acids

Inhibitory nucleic acids of the present disclosure comprise any nucleic acids that can bind a target messenger RNA (mRNA). In some embodiments, an inhibitory nucleic acid of the present disclosure comprises RNA. In some embodiments, an inhibitory nucleic acid of the present disclosure comprises DNA. In some embodiments, an inhibitory nucleic acid of the present disclosure is a DNA/RNA hybrid. In some embodiments, an inhibitory nucleic acid of the present disclosure is an inhibitory RNA. In some embodiments, an inhibitory RNA silences expression of a target gene via RNA interference. In some embodiments, an inhibitory RNA is selected from the group consisting of antisense RNA (asRNA), cis-natural antisense transcript (cis-NAT), CRISPR RNA (crRNA), guide RNA (gRNA), long noncoding RNA (lncRNA), microRNA (miRNA), piwi-interacting RNA (piRNA), small interfering RNA (siRNA), short hairpin RNA (shRNA), trans-acting siRNA (tasiRNA), and repeat associated siRNA (rasiRNA). In some embodiments, an inhibitory RNA is an shRNA.

In some embodiments, an shRNA of the present disclosure comprises a stem-loop structure. In some embodiments, an shRNA of the present disclosure comprises a stem-loop structure flanked by at least 10 bases on either side of the stem. In some embodiments, a stem is double-stranded RNA structure. In some embodiments, a stem is about 30-40 bases in length on each side. In some embodiments, a stem is about 30-39 bases in length on each side. In some embodiments, a stem is about 30-38 bases in length on each side. In some embodiments, a stem is about 30-37 bases in length on each side. In some embodiments, a stem is about 30-36 bases in length on each side. In some embodiments, a stem is about 30-35 bases in length on each side. In some embodiments, a stem is about 30-34 bases in length on each side. In some embodiments, a stem is about 30-33 bases in length on each side. In some embodiments, a stem is about 30-32 bases in length on each side. In some embodiments, a stem is about 30-31 bases in length on each side. In some embodiments, a stem is about 31-40 bases in length on each side. In some embodiments, a stem is about 32-40 bases in length on each side. In some embodiments, a stem is about 33-40 bases in length on each side. In some embodiments, a stem is about 34-40 bases in length on each side. In some embodiments, a stem is about 35-40 bases in length on each side. In some embodiments, a stem is about 36-40 bases in length on each side. In some embodiments, a stem is about 37-40 bases in length on each side. In some embodiments, a stem is about 38-40 bases in length on each side. In some embodiments, a stem is about 39-40 bases in length on each side. In some embodiments, a stem is 35 bases in length on each side. In some embodiments, an shRNA of the present disclosure comprises a scaffold. In some embodiments, an shRNA of the present disclosure comprises a 5′ scaffold. In some embodiments, an shRNA of the present disclosure comprises a 3′ scaffold. In some embodiments, an shRNA of the present disclosure comprises a guide strand. In some embodiments, an shRNA of the present disclosure comprises a passenger strand. In some embodiments, an shRNA of the present disclosure comprises a loop connecting the guide strand and the passenger strand. In some embodiments, a loop is about 8-20 bases in length. In some embodiments, a loop is about 8-19 bases in length. In some embodiments, a loop is about 8-18 bases in length. In some embodiments, a loop is about 8-17 bases in length. In some embodiments, a loop is about 8-16 bases in length. In some embodiments, a loop is about 8-15 bases in length. In some embodiments, a loop is about 8-14 bases in length. In some embodiments, a loop is about 8-13 bases in length. In some embodiments, a loop is about 8-12 bases in length. In some embodiments, a loop is about 8-11 bases in length. In some embodiments, a loop is about 8-10 bases in length. In some embodiments, a loop is about 8-9 bases in length. In some embodiments, a loop is about 9-20 bases in length. In some embodiments, a loop is about 10-20 bases in length. In some embodiments, a loop is about 11-20 bases in length. In some embodiments, a loop is about 12-20 bases in length. In some embodiments, a loop is about 13-20 bases in length. In some embodiments, a loop is about 14-20 bases in length. In some embodiments, a loop is about 15-20 bases in length. In some embodiments, a loop is about 16-20 bases in length. In some embodiments, a loop is about 17-20 bases in length. In some embodiments, a loop is about 18-20 bases in length. In some embodiments, a loop is about 19-20 bases in length. In some embodiments, the sections of the stem closest to the loop comprises a guide strand and a passenger strand. In some embodiments, the sections of the stem furthest from the loop comprises a 5′ scaffold and a 3′ scaffold. In some embodiments, an shRNA of the present disclosure comprises, from 5′ to 3′, a 5′ scaffold, a guide strand, a loop, a passenger strand, and a 3′ scaffold. In some embodiments, an shRNA of the present disclosure comprises, from 5′ to 3′, a 5′ scaffold, a passenger strand, a loop, a guide strand, and a 3′ scaffold.

In some embodiments, a scaffold of the present disclosure comprises an miRNA scaffold. In some embodiments, an miRNA scaffold comprises an miRNA-155 5′ scaffold, an miRNA-155 3′ scaffold, an miRNA-30 5′ scaffold, an miRNA-30 3′ scaffold, an miRNA-16 5′ scaffold, an miRNA-16 3′ scaffold, an miRNA-125 5′ scaffold, an miRNA-125 3′ scaffold, an miRNA-223 5′ scaffold, or an miRNA-223 3′ scaffold.

In some embodiments, a guide strand of the present disclosure is about 19-24 bases in length. In some embodiments, a guide strand of the present disclosure is 19-24 bases in length. In some embodiments, a guide strand is 18 bases in length. In some embodiments, a guide strand is 19 bases in length. In some embodiments, a guide strand is 20 bases in length. In some embodiments, a guide strand is 21 bases in length. In some embodiments, a guide strand is 22 bases in length. In some embodiments, a guide strand is 23 bases in length. In some embodiments, a guide strand is 24 bases in length. In some embodiments, a guide strand is 25 bases in length.

In some embodiments, a passenger strand of the present disclosure is about 17-24 bases in length. In some embodiments, a passenger strand of the present disclosure is about 17-22 bases in length. In some embodiments, a passenger strand of the present disclosure is 17-22 bases in length. In some embodiments, a passenger strand is 17 bases in length. In some embodiments, a passenger strand is 18 bases in length. In some embodiments, a passenger strand is 19 bases in length. In some embodiments, a passenger strand is 20 bases in length. In some embodiments, a passenger strand is 21 bases in length. In some embodiments, a passenger strand is 22 bases in length. In some embodiments, a passenger strand is 23 bases in length. In some embodiments, a passenger strand is 22 bases in length. In some embodiments, a passenger strand is 24 bases in length. In some embodiments, a passenger strand is 1-2 bases shorter than a corresponding guide strand. In some embodiments, a passenger strand is 1 base shorter than a corresponding guide strand. In some embodiments, a passenger strand is 2 bases shorter than a corresponding guide strand. In some embodiments, a passenger strand is the same length as a corresponding guide strand.

In some embodiments, a guide strand of the present disclosure has a G/C content of about 36%-50%. In some embodiments, a guide strand of the present disclosure has a G/C content of 36%-50%. In some embodiments, a guide strand has a G/C content of 35%. In some embodiments, a guide strand has a G/C content of 36%. In some embodiments, a guide strand has a G/C content of 37%. In some embodiments, a guide strand has a G/C content of 38%. In some embodiments, a guide strand has a G/C content of 39%. In some embodiments, a guide strand has a G/C content of 40%. In some embodiments, a guide strand has a G/C content of 41%. In some embodiments, a guide strand has a G/C content of 42%. In some embodiments, a guide strand has a G/C content of 43%. In some embodiments, a guide strand has a G/C content of 44%. In some embodiments, a guide strand has a G/C content of 45%. In some embodiments, a guide strand has a G/C content of 46%. In some embodiments, a guide strand has a G/C content of 47%. In some embodiments, a guide strand has a G/C content of 48%. In some embodiments, a guide strand has a G/C content of 49%. In some embodiments, a guide strand has a G/C content of 50%. In some embodiments, a guide strand has a G/C content of 51%.

In some embodiments, a passenger strand of the present disclosure has a G/C content of about 36%-50%. In some embodiments, a passenger strand of the present disclosure has a G/C content of 36%-50%. In some embodiments, a passenger strand has a G/C content of 35%. In some embodiments, a passenger strand has a G/C content of 36%. In some embodiments, a passenger strand has a G/C content of 37%. In some embodiments, a passenger strand has a G/C content of 38%. In some embodiments, a passenger strand has a G/C content of 39%. In some embodiments, a passenger strand has a G/C content of 40%. In some embodiments, a passenger strand has a G/C content of 41%. In some embodiments, a passenger strand has a G/C content of 42%. In some embodiments, a passenger strand has a G/C content of 43%. In some embodiments, a passenger strand has a G/C content of 44%. In some embodiments, a passenger strand has a G/C content of 45%. In some embodiments, a passenger strand has a G/C content of 46%. In some embodiments, a passenger strand has a G/C content of 47%. In some embodiments, a passenger strand has a G/C content of 48%. In some embodiments, a passenger strand has a G/C content of 49%. In some embodiments, a passenger strand has a G/C content of 50%. In some embodiments, a passenger strand has a G/C content of 51%.

In some embodiments, a stemloop formed by a 5′ scaffold, a guide strand, a passenger strand, and a 3′ scaffold is recognized by Drosha. In some embodiments, Drosha cleaves 10-15 bases above the base of a stemloop. In some embodiments, a Drosha-cleaved stemloop will be recognized by Dicer. In some embodiments, Dicer cleaves 20-24 bases from the 5′ and 3′ ends of the stemloop.

In some embodiments, a guide strand of the present disclosure comprises a nucleic acid sequence that is reverse complementary to a target gene transcript comprising a target nucleic acid sequence. In some embodiments, a passenger strand of the present disclosure comprises a nucleic acid sequence that is not fully complementary to a corresponding guide strand. In some embodiments, a passenger strand comprises a nucleic acid sequence that is not fully complementary to a corresponding guide strand by one nucleotide. In some embodiments, a passenger strand of the present disclosure comprises a nucleic acid sequence that is not fully complementary to a corresponding guide strand by two nucleotides. In some embodiments, a passenger strand of the present disclosure comprises a nucleic acid sequence that is not fully complementary to a corresponding guide strand by three nucleotides. In some embodiments, a passenger strand of the present disclosure comprises a nucleic acid sequence that is not fully complementary to a corresponding guide strand by four nucleotides.

In some embodiments, an shRNA of the present disclosure comprises: (a) a nucleic acid sequence selected from Table 6a, Table 6b, Table 6c, Table 6d, or Table 6e; (b) a nucleic acid sequence that differs from a sequence selected from Table 6a, Table 6b, Table 6c, Table 6d, or Table 6e by no more than 5 substitutions, additions, or deletions; or (c) a nucleic acid sequence that is at least 80% identical to a sequence selected from Table 6a, Table 6b, Table 6c, Table 6d, or Table 6e. In some embodiments, an shRNA of the present disclosure comprises a nucleic acid sequence selected from Table 6a, Table 6b, Table 6c, Table 6d, or Table 6e. In some embodiments, an shRNA of the present disclosure comprises a nucleic acid sequence that differs from a sequence selected from Table 6a, Table 6b, Table 6c, Table 6d, or Table 6e by no more than 5 substitutions, additions, or deletions. In some embodiments, an shRNA of the present disclosure comprises a nucleic acid sequence that is at least 80% identical to a sequence selected from Table 6a, Table 6b, Table 6c, Table 6d, or Table 6e.

In some embodiments, an inhibitory RNA of the present disclosure is or comprises an miRNA. In some embodiments, an miRNA of the present disclosure comprises: (a) a nucleic acid sequence selected from Table 7; (b) a nucleic acid sequence that differs from a sequence selected from Table 7 by no more than 5 substitutions, additions, or deletions; or (c) a nucleic acid sequence that is at least 80% identical to a sequence selected from Table 7. In some embodiments, an miRNA of the present disclosure comprises a nucleic acid sequence selected from Table 7. In some embodiments, an miRNA of the present disclosure comprises a nucleic acid sequence that differs from a sequence selected from Table 7 by no more than 5 substitutions, additions, or deletions. In some embodiments, an miRNA of the present disclosure comprises a nucleic acid sequence that is at least 80% identical to a sequence selected from Table 7.

### Targets of Inhibitory Nucleic Acids

In some embodiments, inhibitory nucleic acids of the present disclosure regulate gene expression in modified immune cells of the present disclosure via RNA interference (RNAi). In some embodiments, inhibitory nucleic acids of the present disclosure target mRNA comprising a complementary sequence. In some embodiments, inhibitory nucleic acids of the present disclosure induce degradation of target mRNA. In some embodiments, inhibitory nucleic acids of the present disclosure repress translation of target mRNA.

In some embodiments, a target gene transcript is mammalian. In some embodiments, a target gene transcript is human. In some embodiments, contacting a target gene transcript with one or more inhibitory nucleic acids (e.g., RNAs) reduces translation of the target gene transcript. In some embodiments, a target gene transcript is expressed in an immune cell. In some embodiments, an immune cell is a stem cell, macrophage, monocyte, or dendritic cell. In some embodiments, a target gene transcript is expressed in a macrophage. In some embodiments, a target gene transcript is expressed in a monocyte. In some embodiments, reduced translation of a target gene transcript is associated with an M1 phenotype. In some embodiments, reduced translation of a target gene transcript is associated with an M2 phenotype. In some embodiments, increased expression of an inhibitory nucleic acid (e.g., RNA) of the present disclosure is associated with an M1 phenotype. In some embodiments, increased expression of an inhibitory nucleic acid (e.g., RNA) of the present disclosure is associated with an M2 phenotype.

In some embodiments, a target gene transcript of the present disclosure comprises: (a) a target nucleic acid sequence selected from Table 8; (b) a target nucleic acid sequence that differs from a sequence selected from Table 8 by no more than 5 substitutions, additions, or deletions; or (c) a target nucleic acid sequence that is at least 80% identical to a sequence selected from Table 8. In some embodiments, a target gene transcript of the present disclosure comprises a target nucleic acid sequence selected from Table 8. In some embodiments, a target gene transcript of the present disclosure comprises a target nucleic acid sequence that differs from a sequence selected from Table 8 by no more than 5 substitutions, additions, or deletions. In some embodiments, a target gene transcript of the present disclosure comprises a target nucleic acid sequence that is at least 80% identical to a sequence selected from Table 8.

In some embodiments, a target gene transcript of the present disclosure encodes human ATG7, C/EBP-alpha, C/EBP-beta, CD36, CLEC1A, FATS, GOLM1, HAVCR2, ITGAD, KLF4, KLF6, LILRB1, LILRB2, LILRB4, MAF, MafB, PD-L1, PIK3CG, PIK3CG, PPARα, PPARγ, PTGS2, SIGLEC7, SIGLEC10, SIRPα, SLC15A3, STAT3, STAT6, TNFRSF1B, TOX, TREM2, YTHDF2, or ZFP36. In some embodiments, a target gene transcript of the present disclosure encodes human SIRPα. In some embodiments, a target gene transcript of the present disclosure encodes human PD-1 or PD-L1.

In some embodiments, an inhibitory nucleic acid of the present disclosure comprises one or more inhibitory nucleic acids. In some embodiments, one or more inhibitory nucleic acids comprise one inhibitory nucleic acid. In some embodiments, one or more inhibitory nucleic acids comprise at least two, three, four or five inhibitory nucleic acids. In some embodiments, one or more inhibitory nucleic acids comprise two inhibitory nucleic acids. In some embodiments, one or more inhibitory nucleic acids comprise three inhibitory nucleic acids. In some embodiments, one or more inhibitory nucleic acids comprise four inhibitory nucleic acids. In some embodiments, one or more inhibitory nucleic acids comprise five inhibitory nucleic acids.

In some embodiments, at least two, three, four, or five inhibitory nucleic acids are in tandem. In some embodiments, at least two, three, four, or five inhibitory nucleic acids comprise identical sequences. In some embodiments, at least two, three, four, or five inhibitory nucleic acids comprise at least two different sequences. In some embodiments, at least three, four, or five inhibitory nucleic acids comprise at least three different sequences. In some embodiments, at least four or five inhibitory nucleic acids comprise at least four different sequences. In some embodiments, at least five inhibitory nucleic acids comprise at least five different sequences. In some embodiments, at least two, three, four, or five inhibitory nucleic acids encode inhibitory RNAs comprising nucleic acid sequences that are reverse complementary to the same target gene transcript. In some embodiments, at least two, three, four, or five inhibitory nucleic acids encode inhibitory RNAs comprising nucleic acid sequences that are reverse complementary to different target gene transcripts.

In some embodiments, at least two, three, four, or five inhibitory nucleic acids encode inhibitory RNAs comprising identical miRNA scaffolds. In some embodiments, at least two, three, four, or five inhibitory nucleic acids encode inhibitory RNAs comprising at least two different miRNA scaffolds. In some embodiments, at least three, four, or five inhibitory nucleic acids encode inhibitory RNAs comprising at least three different miRNA scaffolds. In some embodiments, at least four or five inhibitory nucleic acids encode inhibitory RNAs comprising at least four different miRNA scaffolds. In some embodiments, at least five inhibitory nucleic acids encode inhibitory RNAs comprising at least five different miRNA scaffolds. In some embodiments, at least two different miRNA scaffolds are selected from the group consisting of an miRNA-155 5′ scaffold, an miRNA-155 3′ scaffold, an miRNA-30 5′ scaffold, and an miRNA-30 3′ scaffold.

### Methods of Designing Inhibitory Nucleic Acids

The present disclosure also provides methods of designing inhibitory nucleic acids of the present disclosure. In some embodiments, the present disclosure provides methods of designing inhibitory RNA of the present disclosure. In some embodiments, the present disclosure provides methods of designing shRNA of the present disclosure. In some embodiments, methods of designing an shRNA comprising a guide strand, a passenger strand, and a loop, comprise the steps of: (a) selecting a target region of a target gene transcript, (b) designing the guide strand, and (c) designing the passenger strand. In some embodiments, a guide strand is a reverse complement of a target region. In some embodiments, a guide strand comprises, from 5′ to 3′, an adenine or uracil at position 1, an adenine, guanine, or cytosine at position 10, and a G/C content between 36% and 45% that is increases from 5′ to 3′. In some embodiments, a guide strand comprises a higher G/C content of the 10 bases at the 3′ end of the guide strand relative to the G/C content of the 10 bases at the 5′ end of the guide strand. In some embodiments, a passenger strand comprises the sequence of a target region, from 5′ to 3′, with the following variations: (i) deletion of a nucleotide at position 7 relative to the loop, (ii) deletion of a nucleotide at position 11 relative to the loop, and (iii) mutation of an adenine to guanine and/or mutation of a cytosine to uracil near the deletions at positions 7 and 11, wherein the mutations create two partial G-U base pairs between the passenger strand and the guide strand. In some embodiments, a passenger strand is 2 base pairs shorter than a guide strand. When nucleotide positions are counted relative to a loop, position 1 is nearest the loop and position counts increase moving away from the loop.

In some embodiments, methods of designing an shRNA comprising a guide strand, a passenger strand, and a loop, comprise the steps of: (a) selecting a target region of a target gene transcript, (b) designing the guide strand, wherein the guide strand is a reverse complement of the target region, and wherein the guide strand comprises, from 5′ to 3′, an adenine or uracil at position 1, an adenine, guanine, or cytosine at position 10, and a G/C content between 36% and 45% that is increases from 5′ to 3′; and (c) designing the passenger strand, wherein the passenger strand comprises the sequence of the target region, from 5′ to 3′, with the following variations: (i) deletion of a nucleotide at position 7 relative to the loop, (ii) deletion of a nucleotide at position 11 relative to the loop, (iii) mutating an adenine to guanine and/or mutating a cytosine to uracil near the deletions at positions 7 and 11, wherein the mutations create two partial G-U base pairs between the passenger strand and the guide strand; and wherein the passenger strand is 2 base pairs shorter than the guide strand. In some embodiments, methods of designing an shRNA comprise a target region of a target gene transcript that is 21-22 base pairs in length.

### Pharmaceutical Compositions

The present disclosure, among other things, provides pharmaceutical compositions comprising modified immune cells (e.g., stem cells, macrophages, monocytes, or dendritic cells) comprising one or more of CARs described herein in combination with one or more pharmaceutically or physiologically acceptable carriers, diluents, or excipients. The present disclosure, among other things, also provides pharmaceutical compositions comprising nucleic acids encoding one or more CARs described herein in combination with one or more pharmaceutically or physiologically acceptable carriers, diluents, or excipients.

When “a therapeutically effective amount, “an immunologically effective amount,” “an anti-immune response effective amount,” or “an immune response-inhibiting effective amount” is indicated, a precise amount of a pharmaceutical composition described herein can be determined by a physician with consideration of individual differences in age, weight, immune response, and condition of the patient (subject).

Pharmaceutical compositions described herein may comprise buffers, such as neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); serum and preservatives, such as cryoprotectant. In some embodiments, a pharmaceutical composition is substantially free of contaminants, e.g., there are no detectable levels of a contaminant (e.g., an endotoxin).

Pharmaceutical compositions described herein may be administered in a manner appropriate to the disease, disorder, or condition to be treated or prevented. Quantity and frequency of administration will be determined by such factors as condition of a patient, and type and severity of a patient's disease, disorder, or condition, although appropriate dosages may be determined by clinical trials.

Pharmaceutical compositions described herein may be in a variety of forms. These include, for example, liquid, semi-solid and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for intravenous infusion or injection. In some embodiments, a pharmaceutical composition described herein is formulated for intramuscular or subcutaneous injection. Pharmaceutical compositions described herein can be formulated for administered by using infusion techniques that are commonly known in immunotherapy (See, e.g., Rosenberg et al., New Eng. J. of Med. 319:1676, 1988, which is hereby incorporated by reference in its entirety).

As used herein, the terms “parenteral administration” and “administered parenterally” refer to modes of administration other than enteral and topical administration, usually by injection or infusion, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoral, and intrasternal injection and infusion.

Pharmaceutical compositions comprising modified immune cells described herein may be administered at a dosage of about 10⁴ to about 10⁹ cells/kg body weight (e.g., about 10⁵ to about 10⁶ cells/kg body weight), including all integer values within those ranges. In some embodiments, a dose of immune cells described herein (e.g., stem cells, macrophages, monocytes, or dendritic cells) comprises at least about 1×10⁶, about 1.1×10⁶, about 2×10⁶, about 3.6×10⁶, about 5×10⁶, about 1×10⁷, about 1.8×10⁷, about 2×10⁷, about 5×10⁷, about 1×10⁸, about 2×10⁸, about 5×10⁸, about 1×10⁹, about 2×10⁹, or about 5×10⁹ cells. Pharmaceutical compositions described herein may also be administered multiple times at a certain dosage. An optimal dosage and treatment regime for a particular patient can readily be determined by one skilled in the art by monitoring a patient for signs of a disease, disorder, or condition and adjusting treatment accordingly.

It may be desired to administer pharmaceutical compositions described herein to a subject and then subsequently redraw blood (or have apheresis performed), activate collected immune cells, and reinfuse a subject with activated immune cells. This process can be performed multiple times, e.g., every few weeks. Immune cells (e.g., stem cells, macrophages, monocytes, or dendritic cells) can be activated from blood draws of from about 10 cc to about 400 cc. In some embodiments, immune cells (e.g., macrophages, monocytes, or dendritic cells) are activated from blood draws of about 20 cc, about 30 cc, about 40 cc, about 50 cc, about 60 cc, about 70 cc, about 80 cc, about 90 cc, or about 100 cc. Without being bound by theory, methods comprising multiple blood draw and reinfusions described herein may select for certain immune cell populations.

In some embodiments, pharmaceutical compositions described herein are administered in combination with (e.g., before, simultaneously, or following) a second therapy. For example, a second therapy can include, but is not limited to antiviral therapy (e.g., cidofovir, interleukin-2, Cytarabine (ARA-C), or natalizumab), chimeric antigen receptor-T cell (CAR-T) therapy, T-cell receptor (TCR)-T cell therapy, chemotherapy, radiation, an immunosuppressive agent (e.g., cyclosporin, azathioprine, methotrexate, mycophenolate, FK506 antibody, or glucocorticoids), an antagonist (e.g., one or more of a PD-1 antagonist, a PD-L1 antagonist, CTLA4 antagonist, CD47 antagonist, SIRPa antagonist, CD40 agonists, CSF1/CSF1R antagonist, or a STING agonist), or an immunoablative agent (e.g., an anti-CD52 antibody (e.g., alemtuzumab), an anti-CD3 antibody, cytoxin, fludaribine, cyclosporin, FK506, rapamycin, mycophenolic acid, a steroid, FR901228, or irradiation.

In some embodiments, pharmaceutical compositions described herein are administered in combination with (e.g., before, simultaneously, or following) bone marrow transplantation or lymphocyte ablative therapy using a chemotherapy agent (e.g., fludarabine, external-beam radiation therapy (XRT), cyclophosphamide, or Rituxan). In certain embodiments, subjects undergo standard treatment with high dose chemotherapy followed by peripheral blood stem cell transplantation. In certain embodiments, following transplant, subjects receive an infusion of a pharmaceutical composition comprising immune cells described herein. Pharmaceutical compositions described herein may be administered before or following surgery.

A dosage of any aforementioned therapy to be administered to a subject will vary with a disease, disorder, or condition being treated and based on a specific subject. Scaling of dosages for human administration can be performed according to art-accepted practices. For example, a dose of alemtuzumab will generally be about 1 mg to about 100 mg for an adult, usually administered daily for a period of between about 1 day to about 30 days, e.g., a daily dose of about 1 mg to about 10 mg per day (e.g., as described in U.S. Pat. No. 6,120,766, which is hereby incorporated by reference in its entirety).

### Methods of Treatment

The present disclosure, among other things, provides methods of treating a disease or disorder (e.g., a disease or a disorder described herein) in a subject comprising delivering a pharmaceutical composition described herein. In some embodiments, a therapeutically effective amount of a pharmaceutical composition described herein is administered to a subject having a disease or disorder. Pharmaceutical compositions described herein can be for use in the manufacture of a medicament for treating a disease or disorder in a subject or stimulating an immune response in a subject.

A subject to be treated with methods described herein can be a mammal, e.g., a primate, e.g., a human (e.g., a patient having, or at risk of having, a disease or disorder described herein). In some embodiments, modified immune cells (e.g., stem cells, macrophages, monocytes, or dendritic cells) may be autologous, allogeneic, or xenogeneic with respect to a subject. Pharmaceutical compositions described herein can be administered to a subject in accordance with a dosage regimen described herein, alone or in combination with one or more therapeutic agents, procedures, or modalities.

Pharmaceutical compositions described herein can be used to treat or prevent a disease associated with a tumor or cancer.

A method of treating (e.g., one or more of reducing, inhibiting, or delaying progression of) a cancer or a tumor in a subject with a pharmaceutical composition described herein is provided. A subject can have an adult or pediatric form of cancer. A cancer may be at an early, intermediate, or late stage, or a metastatic cancer. A cancer can include, but is not limited to, a solid tumor, a hematological cancer (e.g., leukemia, lymphoma, or myeloma, e.g., multiple myeloma), or a metastatic lesion. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma, e.g., a cutaneous melanoma), pancreas, and bones (e.g., a chordoma).

In some embodiments, a cancer is selected from a lung cancer (e.g., a non-small cell lung cancer (NSCLC) (e.g., a non-small cell lung cancer (NSCLC) with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, a thyroid cancer (e.g., anaplastic thyroid carcinoma), a cervical cancer (e.g., a squamous cell carcinoma of the cervix), a neuroendocrine tumor (NET) (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hodgkin lymphoma), a myeloma (e.g., a multiple myeloma), or a leukemia (e.g., a myeloid leukemia or a lymphoid leukemia).

In some embodiments, a cancer is a brain tumor, e.g., a glioblastoma, a gliosarcoma, or a recurrent brain tumor. In some embodiments, a cancer is a pancreatic cancer, e.g., an advanced pancreatic cancer. In some embodiments, a cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, a cancer is a renal cancer, e.g., a renal cell carcinoma (RCC) (e.g., a metastatic renal cell carcinoma). In some embodiments, a cancer is a breast cancer, e.g., a metastatic breast carcinoma or a stage IV breast carcinoma, e.g., a triple negative breast cancer (TNBC). In some embodiments, a cancer is a virus-associated cancer. In some embodiments, a cancer is an anal canal cancer (e.g., a squamous cell carcinoma of the anal canal). In some embodiments, a cancer is a cervical cancer (e.g., a squamous cell carcinoma of the cervix). In some embodiments, a cancer is a gastric cancer (e.g., an Epstein Barr Virus (EBV) positive gastric cancer, or a gastric or gastro-esophageal junction carcinoma). In some embodiments, a cancer is a head and neck cancer (e.g., an HPV positive and negative squamous cell cancer of the head and neck (SCCHN)). In some embodiments, a cancer is a nasopharyngeal cancer (NPC). In some embodiments, a cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer.

In some embodiments, a cancer is a hematological cancer. In some embodiments, a cancer is a leukemia, e.g., acute myeloid leukemia, chronic myeloid leukemia, acute lymphoblastic leukemia, chronic lymphocytic leukemia, chronic leukemia, or acute leukemia. In some embodiments, a cancer is a lymphoma, e.g., Hodgkin lymphoma (HL), non-Hodgkin's lymphoma, lymphocytic lymphoma, or diffuse large B cell lymphoma (DLBCL) (e.g., a relapsed or refractory HL or DLBCL). In some embodiments, a cancer is a myeloma, e.g., multiple myeloma.

Pharmaceutical compositions described herein can be used to enhance or modulate an immune response in a subject. In one embodiment, a pharmaceutical composition described herein enhances, stimulates, or increases an immune response in a subject (e.g., a subject having, or at risk of, a disease or disorder described herein). In certain embodiments, a subject is, or is at risk of being, immunocompromised. For example, a subject is undergoing or has undergone a chemotherapeutic treatment and/or radiation therapy.

Administration of pharmaceutical compositions described herein may be carried out in any convenient manner (e.g., injection, ingestion, transfusion, inhalation, implantation, or transplantation). In some embodiments, a pharmaceutical compositions described herein is administered by injection or infusion. Pharmaceutical compositions described herein may be administered to a patient transarterially, subcutaneously, intravenously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by intravenous infusion or injection. In some embodiments, a pharmaceutical composition described herein is administered by intramuscular or subcutaneous injection. Pharmaceutical compositions described herein may be injected directly into a site of inflammation, a local disease site, a lymph node, an organ, a tumor, or site of infection in a subject.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. An example is provided for illustrative purposes only. It is not to be construed as limiting the scope or content of the disclosure in any way.

### EXAMPLES

The following examples are provided so as to describe to the skilled artisan how to make and use methods and compositions described herein, and are not intended to limit the scope of the present disclosure.

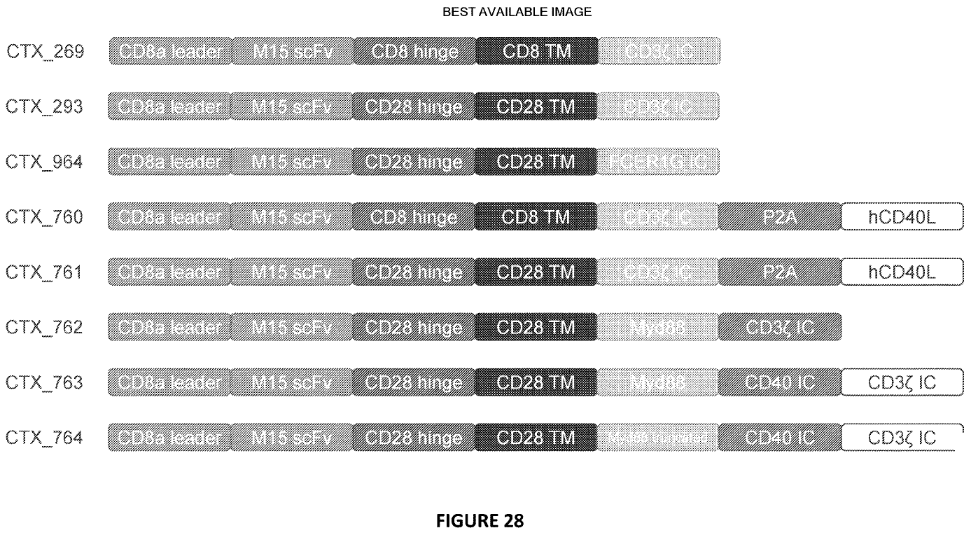

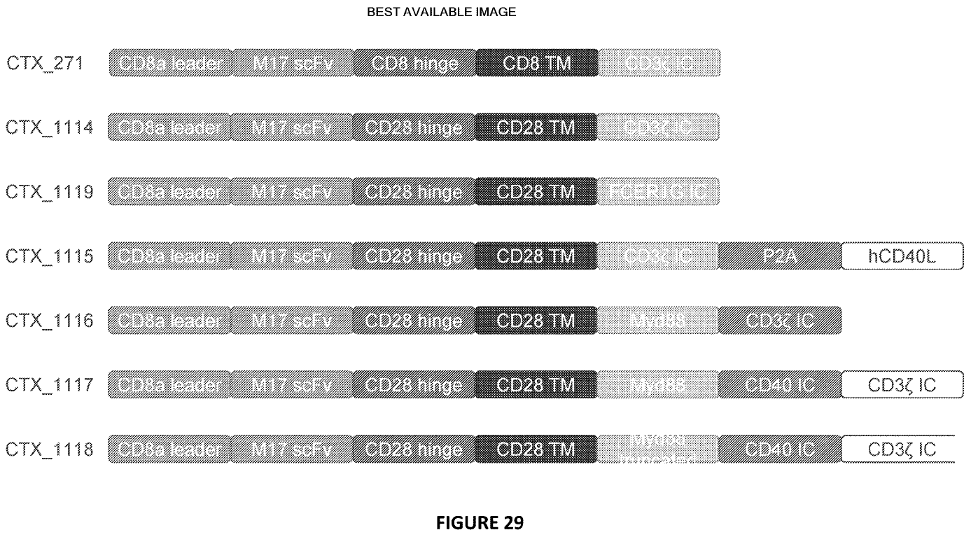

As shown below, Table 1 includes exemplified CAR constructs described herein. FIGS. 28 and 29 show schematics of these exemplary CAR constructs.

TABLE 1
Exemplified CAR Constructs Described Herein

| CTX ID | Constructs |
|---|---|
| CTX_269 | CD8L - M15scFv - CD8H - CD8TM - CD3ζIC |
| CTX_293 | CD8L - M15scFv - CD28H - CD28TM - CD3ζIC |
| CTX_964 | CD8L - M15scFv - CD28H - CD28TM - FCER1γIC |
| CTX_760 | CD8L - M15scFv - CD8H - CD8TM - CD3ζIC - P2A - hCD40L |
| CTX_761 | CD8L - M15scFv - CD28H - CD28TM - CD3ζIC - P2A - hCD40L |
| CTX_762 | CD8L - M15scFv - CD28H - CD28TM - Myd88IC - CD3ζIC |
| CTX_763 | CD8L - M15scFv - CD28H - CD28TM - Myd88IC - CD40IC - CD3ζIC |
| CTX_764 | CD8L - M15scFv - CD28H - CD28TM - Myd88 truncated IC - CD40IC - CD3ζIC |
| CTX_1120 | CD8L - M15scFv - CD28H - CD28TM - Myd88 truncated IC - CD40IC - FCER1γIC |
| CTX_271 | CD8L - M17scFv - CD8H - CD8TM - CD3ζIC |
| CTX_1114 | CD8L - M17scFv - CD28H - CD28TM - CD3ζIC |
| CTX_1115 | CD8L - M17scFv - CD28H - CD28TM - CD3ζIC - P2A - hCD40L |
| CTX_1116 | CD8L - M17scFv - CD28H - CD28TM - Myd88IC - CD3ζIC |
| CTX_1117 | CD8L - M17scFv - CD28H - CD28TM - Myd88IC - CD40IC - CD3ζIC |
| CTX_1118 | CD8L - M17scFv - CD28H - CD28TM - Myd88 truncated IC - CD40IC - CD3ζIC |
| CTX_1119 | CD8L - M17scFv - CD28H - CD28TM - FCER1γIC |
| CTX_001 | CD8L - 4D5scFv - CD8H - CD8TM - CD3ζIC |
| CTX_219 | CD8L - 4D5scFv - CD28H - CD28TM - CD3ζIC |
| CTX_1461 | CD8L - M15scFv - CD28H - CD28TM - FCER1γIC + intronic shRNA against SIRPα |

### Example 1: Anti-Mesothelin Binder Screening

The present Example assesses 20 different anti-mesothelin binders (M1-M20) that were used in a CD8 framework CAR. All 20 CARs were manufactured as 5-methoxyuridine (5moU) mRNA. The CAR mRNA was electroporated into human primary macrophages using a MaxCyte Atx instrument. 24 hours post-electroporation, CAR expression was evaluated via binding of biotinylated mesothelin followed by APC-streptavidin. SS1-based CAR adenovirus was used as a positive control for mesothelin staining and CAR001 (an anti-HER2 CAR) mRNA was used as a positive control for electroporation and mRNA quality.

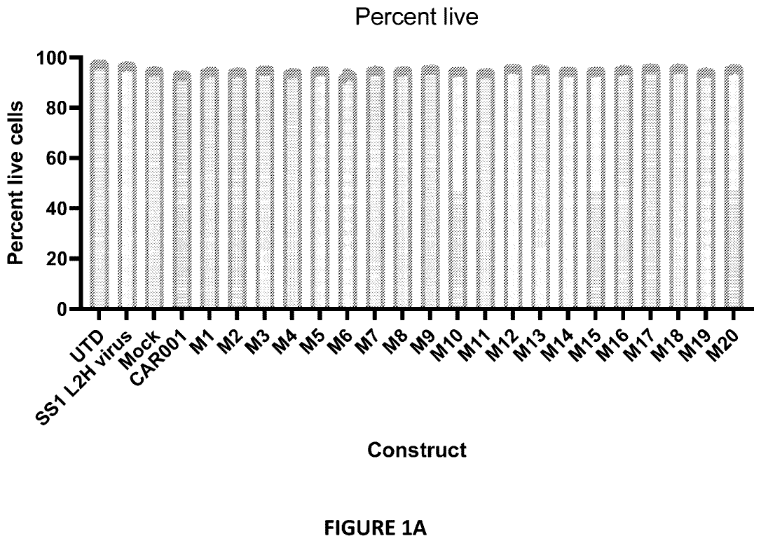

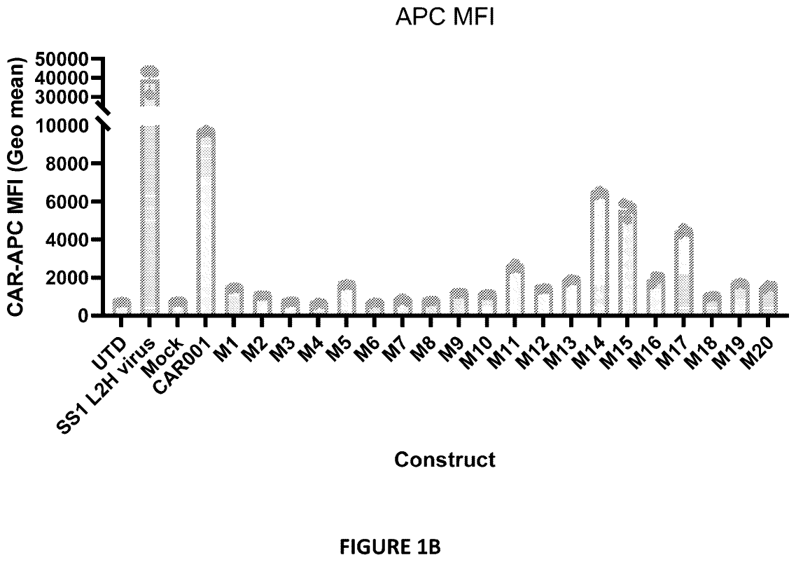

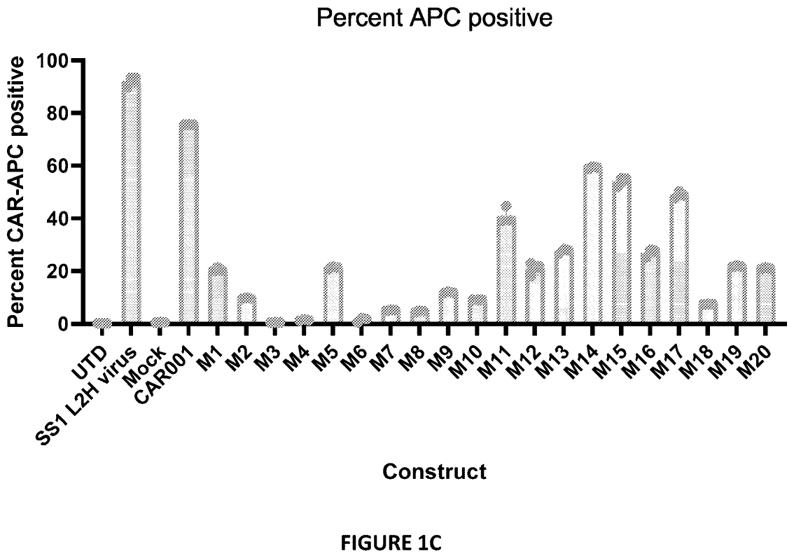

As shown in FIGS. 1A, 1B, and 1C, electroporation of macrophages with mRNA encoding CARs did not negatively impact cell viability. Additionally, as illustrated in FIG. 1B and FIG. 1C, out of the 20 different anti-mesothelin CARs tested, M14, M15, and M17 had the best expression in macrophages.

### Example 2: Killing and Cytokine Secretion of Macrophages Expressing Anti-Mesothelin Binders

The present Example assesses four different anti-mesothelin binders (M11, M14, M15, and M17) that were used in a CD8 framework CAR. All four CARs were manufactured as 5-methoxyuridine (5moU) mRNA. The CAR mRNA was electroporated into human primary macrophages using a MaxCyte ATx instrument. 24 hours post-electroporation, cytokine release was evaluated using a 24 hour co-incubation with K562 WT (mesothelin negative) or K562 MESO (mesothelin positive) cells at a 1:1 ratio of effector cells to target cells (E:T). Killing was also evaluated 24 hours post-electroporation by measuring the change in GFP fluorescence over 72 hours on an Incucyte S3 Live-Cell Analysis System. SS1-based CAR adenovirus was used as a positive control for killing and cytokine release and CAR001 (an anti-HER2 CAR) mRNA was used as a non-mesothelin targeting CAR control.

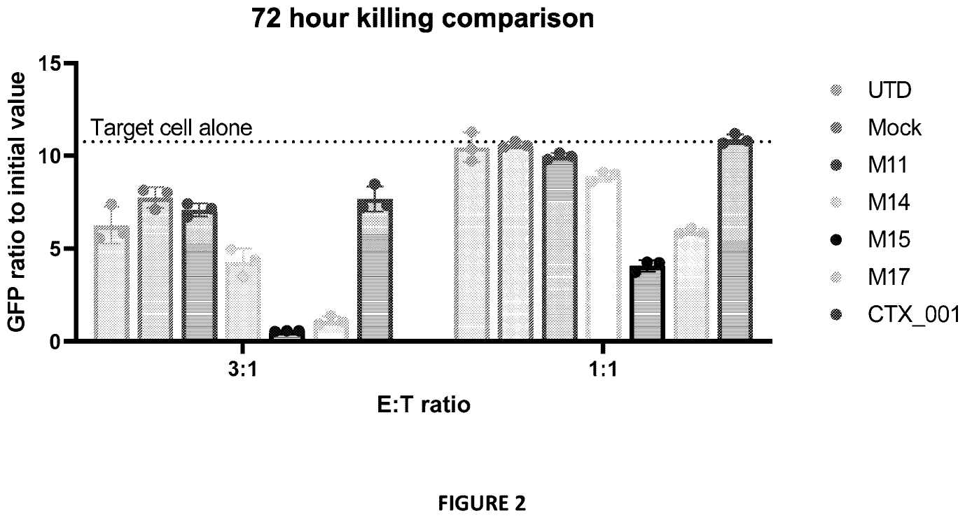

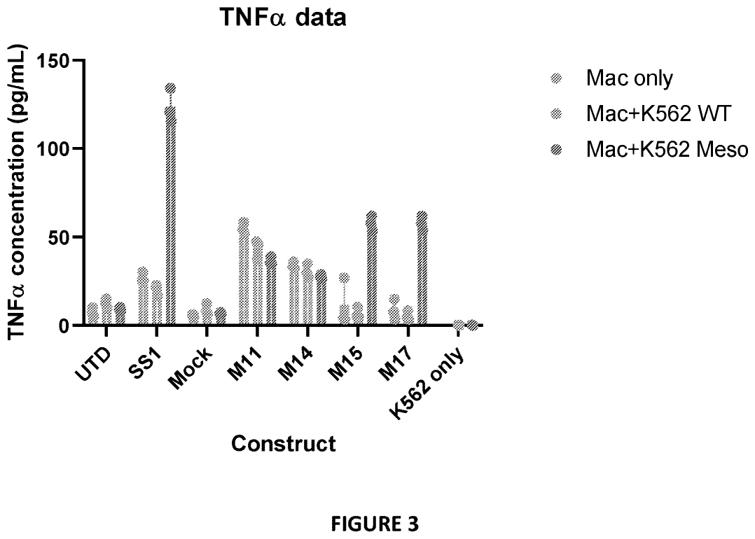

As shown in FIG. 2, out of the four anti-mesothelin binders tested (M11, M14, M15 and M17), only M15 and M17 mediated target cell killing. When macrophages expressing the exemplary anti-mesothelin CARs were co-incubated with K562 cells, the M15 and M17 CAR macrophages mediated TNFα cytokine release from the mesothelin-positive K562 target cells. These data are shown in FIG. 3.

### Example 3: Phagocytosis Screening of Macrophages Expressing Anti-Mesothelin Binders

The present Example assesses four different anti-mesothelin binders (M11, M14, M15, and M17) that were used in a CD8 framework CAR. All four CARs were manufactured as 5-methoxyuridine (5moU) mRNA. The CAR mRNA was electroporated into human primary macrophages using a MaxCyte ATx instrument. 24 hours post-electroporation, phagocytosis was evaluated by 4 hour co-incubation with K562 WT (mesothelin negative) or K562 MESO (mesothelin positive) at 1:1 E:T. At the end time point, cells were analyzed via FACS and CD11β+/GFP+ events were defined as phagocytosis. An adenovirus comprising a CD8− framework SS1 anti-mesothelin scFv CAR (SS1 virus) was used as a positive control for phagocytosis and CAR001 (an anti-HER2 CAR) mRNA was used as a non-mesothelin targeting CAR control.

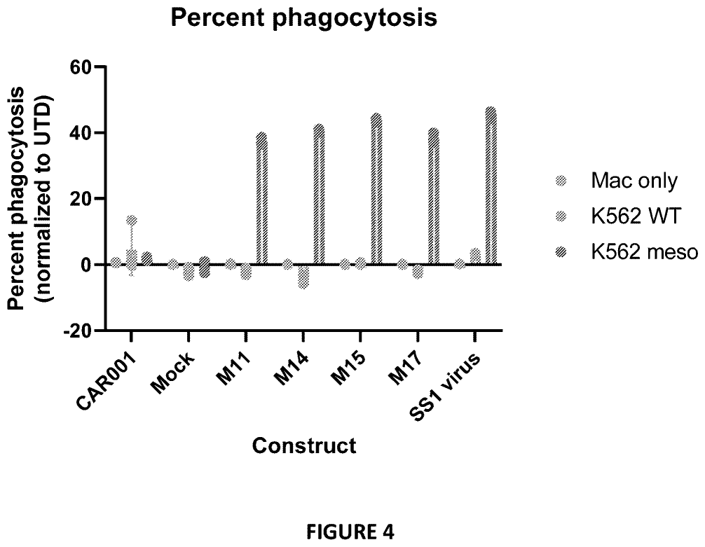

As shown in FIG. 4 macrophages expressing all four anti-mesothelin binders (M11, M14, M15, and M17) mediated phagocytosis of mesothelin-positive target cells.

### Example 4: Phenotyping of Macrophages Transduced with CTX_269

The present Example assesses the phenotype of macrophages transduced with CTX_269. Previously frozen macrophages from three donors were thawed and transduced with varying exemplary MOIs of Ad5f35 vector comprising CTX_269. After 48 hours, cells were analyzed via FACS for viability, CAR expression, and M1/M2 polarization.

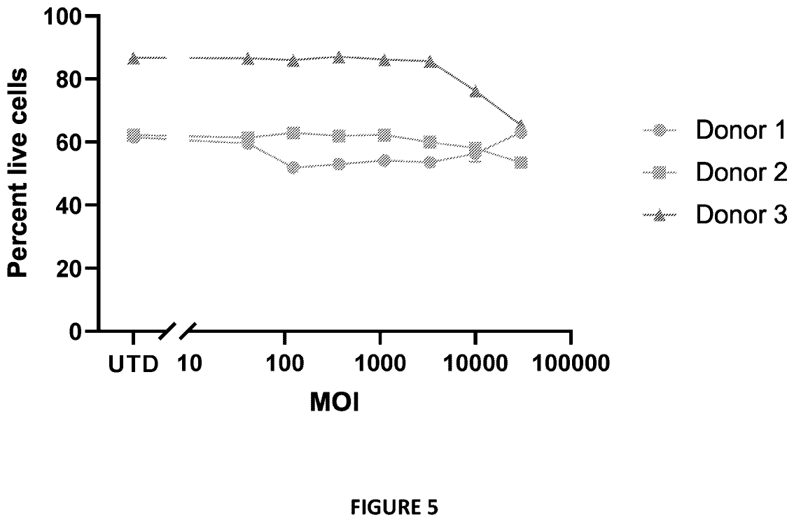

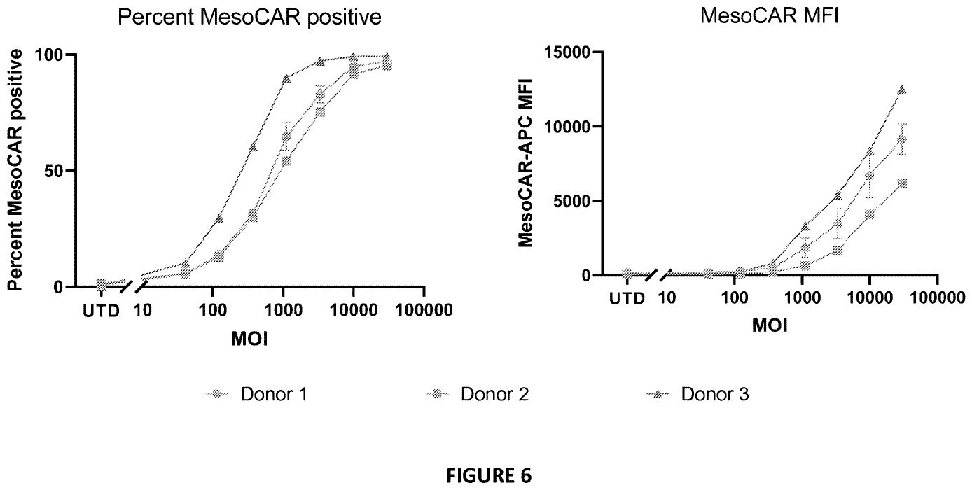

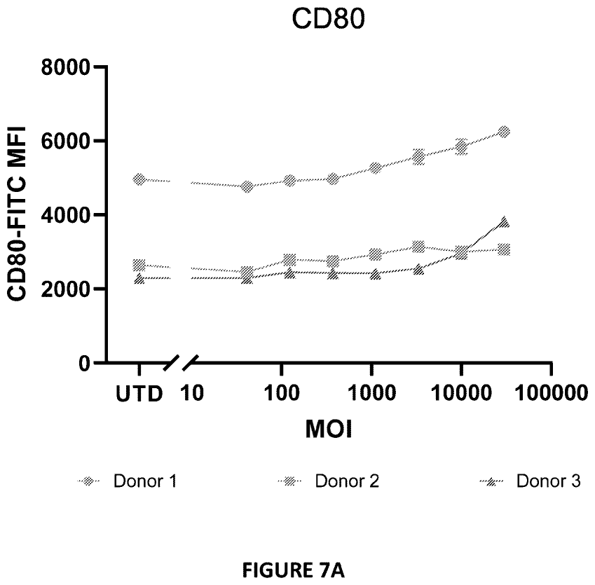

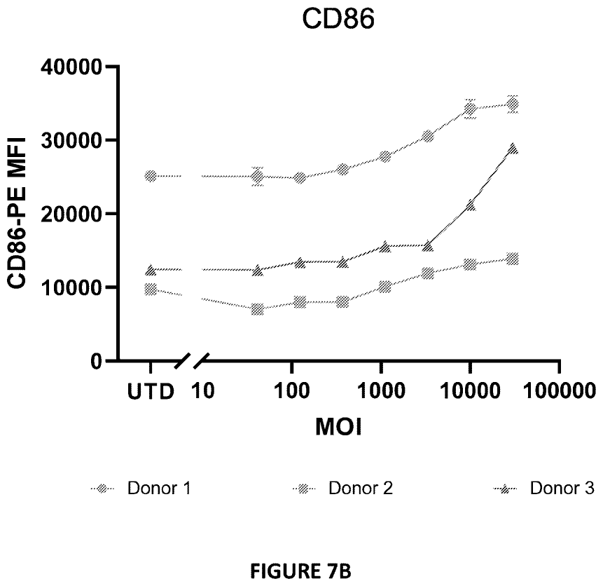

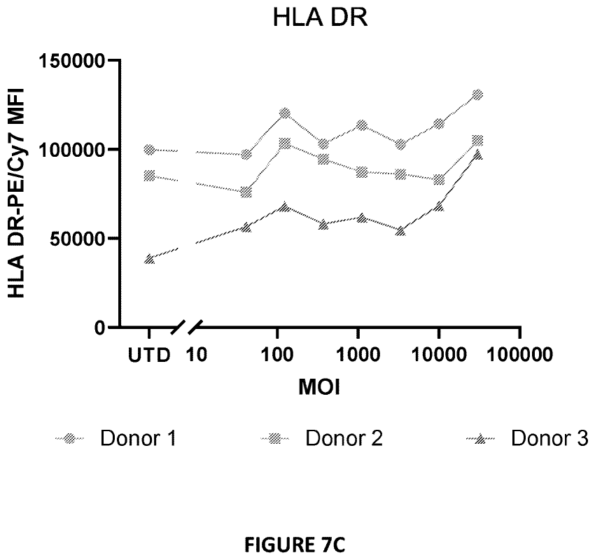

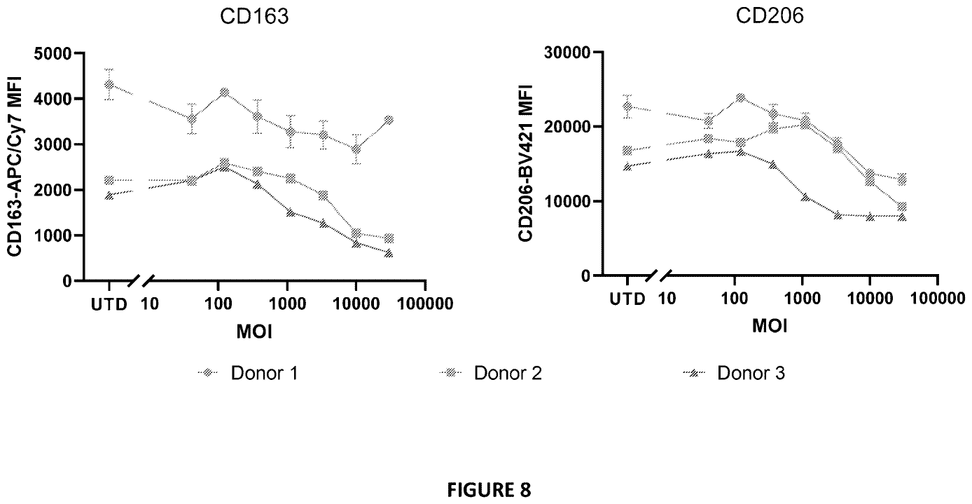

As shown in FIG. 5, Ad5f35 vectors comprising CTX_269 did not significantly impact viability across the MOIs tested. Additionally, as shown in FIG. 6, anti-mesothelin CAR (CTX_269) expression was robust and titratable based on viral MOI. FIGS. 7A, 7B, and 7C show that infection of macrophages with Ad5f35 vectors comprising CTX_269 induced upregulation of exemplary M1-associated markers CD80, CD86, and HLA-DR. M1 marker expression increased with increasing adenoviral MOI. In addition to the upregulation of M1-associated markers, infection of macrophages with Ad5f35 vector comprising CTX_269 induced downregulation of exemplary M2-associated markers CD163 and CD206 (shown in FIG. 8). M2 marker expression decreased with increasing adenoviral MOI.

### Example 5: Resistance of Macrophages Transduced with an Anti-Mesothelin Binder to M2 Cytokines

The present Example assesses the resistance of macrophages transduced with CTX_269 to M2 cytokines. Previously frozen macrophages from three donors were thawed and transduced with 3000 MOI of Ad5f35 vector comprising CTX_269. An anti-HER2 CAR delivered via Ad5f35 (CT0508) at an MOI of 3000 served as a positive control. After 48 hours, Ad5f35 virus was removed from the macrophage cultures. 72 hours post-transduction, media containing 10 ng/mL IL10 was added to the macrophages. 24 hours after cytokine (1L10) addition, macrophages were phenotyped for M1/M2 marker expression.

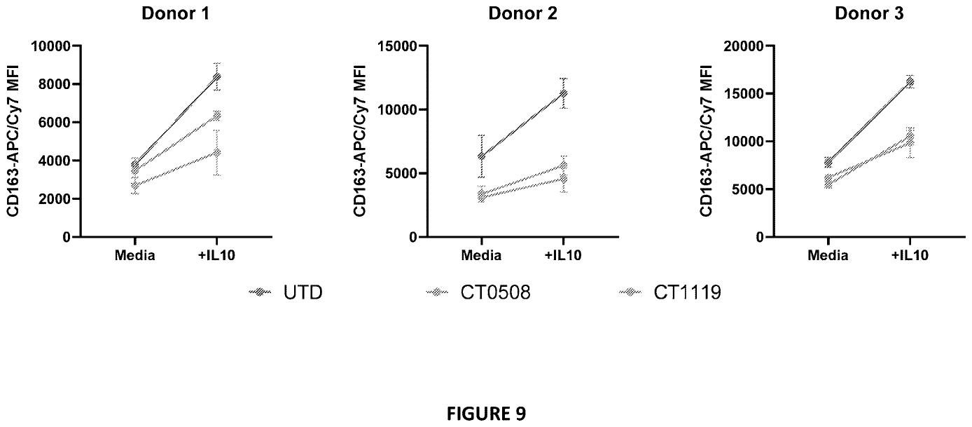

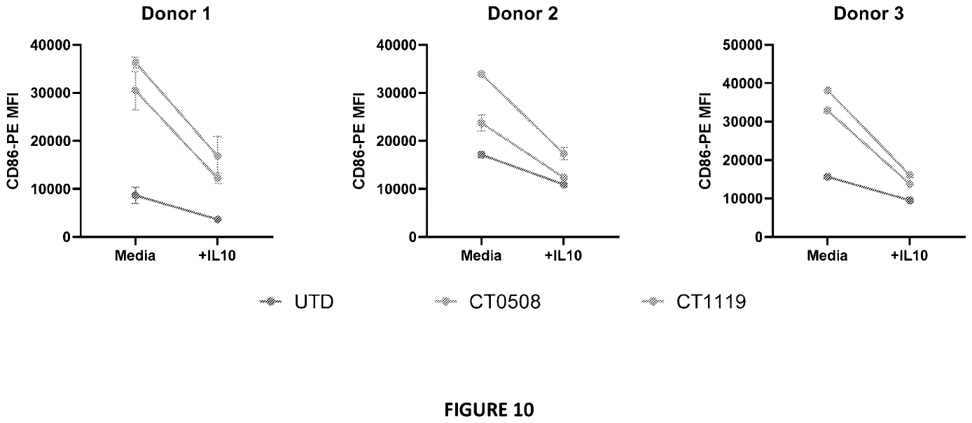

As shown in FIG. 9, macrophages transduced with CTX_269 exhibited a reduced upregulation of CD163 relative to untransduced (UTD) controls when exposed to IL10, indicating a less M2-like phenotype. Similarly, as shown in FIG. 10, macrophages transduced with CTX_269 exhibited a reduced downregulation of CD86 relative to untransduced (UTD) controls when exposed to IL10, suggesting maintenance of the M1 phenotype.

### Example 6: Changes in M1/M2 Polarization of Macrophages Transduced with an Anti-Mesothelin Binder after Stimulation with Mesothelin

The present Example assesses the changes in M1/M2 polarization of macrophages transduced with CTX_269 after exposure to mesothelin. Previously frozen macrophages from two donors were thawed and transduced with 3000 MOI of Ad5f35 vector comprising CTX_269. Macrophages were plated on plates containing exemplary titrations of mesothelin. After 24 hours, macrophages were lifted and analyzed for M1/M2 polarization via flow cytometry.

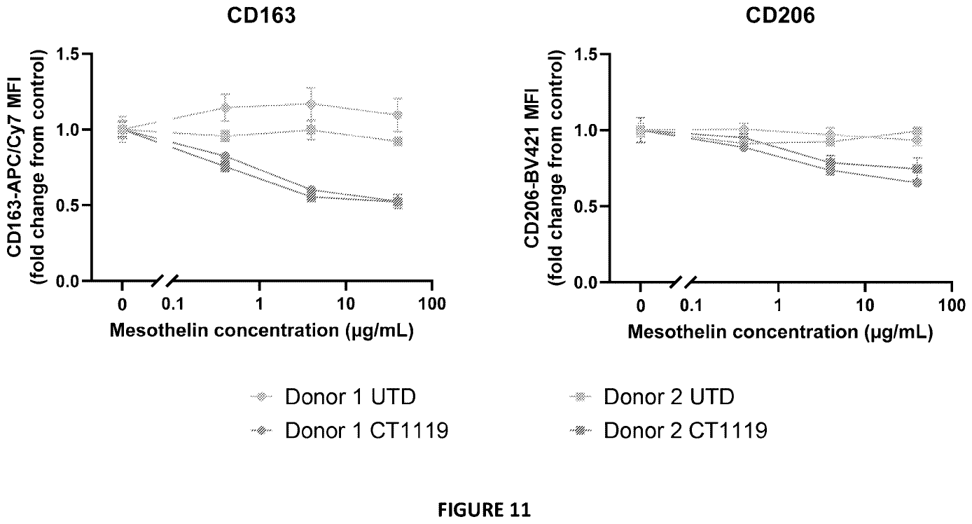

As shown in FIG. 11, exposure of macrophages expressing anti-mesothelin CARs to mesothelin decreased expression of M2-associated markers CD163 and CD206 relative to untransduced (UTD) control cells.

### Example 7: Phagocytosis Assay of Macrophages Transduced with an Anti-Mesothelin Binder

The present Example assesses the phagocytosis ability of macrophages transduced with CTX_269. Previously frozen macrophages from two donors were thawed and transduced with 3000 MOI of Ad5f35 vector comprising CTX_269. After 48 hours, the macrophages were lifted and placed onto a 96 well plate (50,000 cells/well). Wild-type (no mesothelin expression) and mesothelin-expressing target cells were stained with pHrodo (a pH sensitive dye). Macrophages and target cells were mixed at a 1:1 E:T ratio. Fluorescence was tracked in an Incucyte S3 Live-Cell Analysis System for 24 hours, with imaging occurring every hour. Phagocytosis was determined by calculating the area under the curve (AUC) of the fluorescence values at each time point.

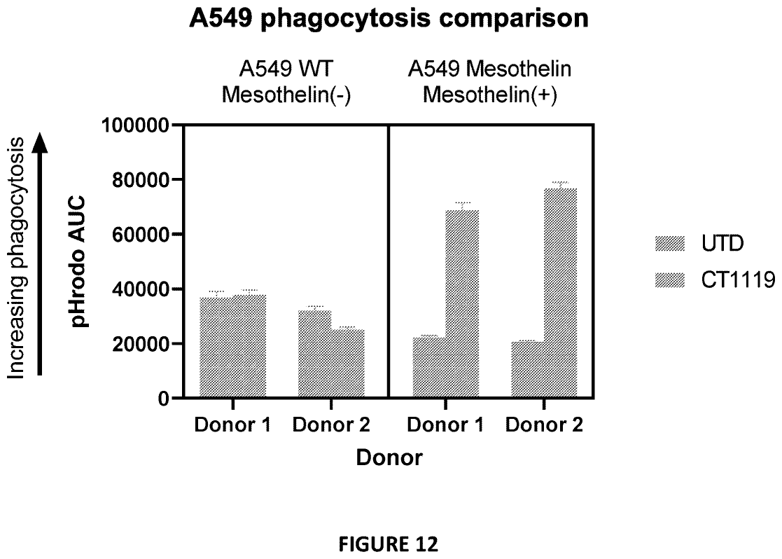

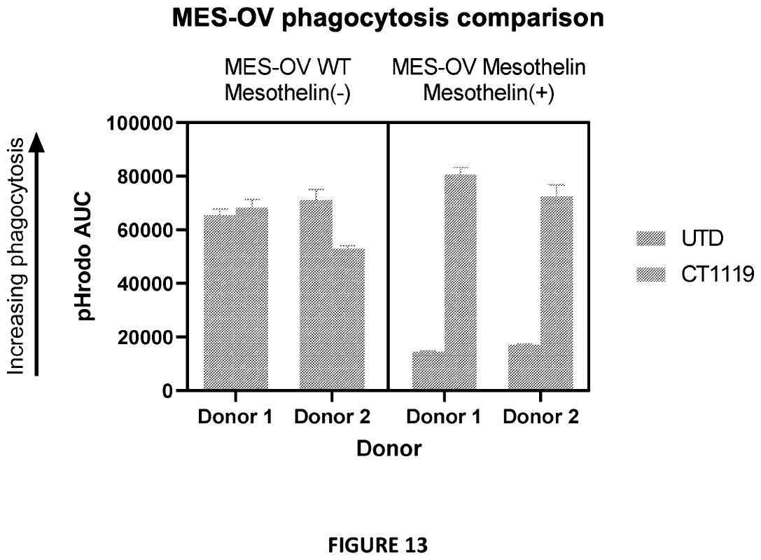

As shown in FIG. 12, CTX_269 transduced macrophages only show increased phagocytosis of mesothelin-positive cells, indicating that phagocytosis of A549 lung adenocarcinoma cells by macrophages transduced with CTX_269 involved mesothelin expression by the target cells. FIG. 13 shows that, in the phagocytosis assay with MES_OV ovarian cystadenocarcinoma cells, there was increased phagocytosis of target cells expressing mesothelin by macrophages transduced with CTX_269 relative to untransduced (UTD) control macrophages. Such increased phagocytosis was not seen in wild-type ovarian cystadenocarcinoma that did not express mesothelin.

### Example 8: Killing of Tumor Cell Lines with Macrophages Transduced with an Anti-Mesothelin Binder

The present Example assesses the killing ability of macrophages transduced with CTX_269. Previously frozen macrophages from three donors were thawed and transduced with 3000 MOI of Ad5f35 vector comprising CTX_269. After 48 hours, the macrophages were lifted and placed into a 96 well plate at various E:T ratios. Mesothelin/mKate2-expressing target cells were plated at a constant 10,000 cells per well. Fluorescence was tracked on an Incucyte S3 Live-Cell Analysis System for 72 hours, with imaging occurring every 4 hours. Killing of target cells was determined by calculating the fluorescence at 72 hours divided by the initial fluorescence.

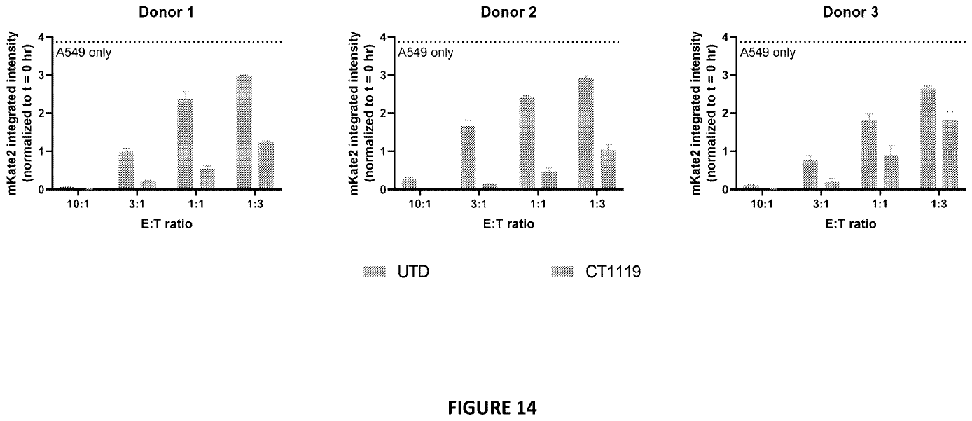

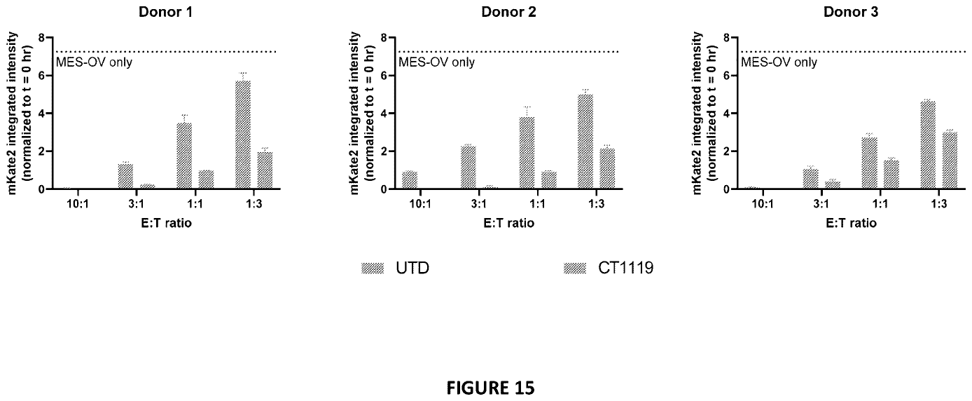

As shown in FIG. 14, CTX_269 transduced macrophages exhibited enhanced killing of mesothelin-expressing A549 lung adenocarcinoma cells compared to transduced (UTD) macrophages. Additionally, FIG. 15 shows that CTX_269 transduced macrophages exhibited enhanced killing of mesothelin-expressing MES_OV ovarian cystadenocarcinoma cells compared to UTD macrophages.

### Example 9A: Cytokine Release from Macrophages Transduced with an Anti-Mesothelin Binder after Exposure to Mesothelin

The present Example assesses the cytokine release of macrophages transduced with CTX_269 after exposure to mesothelin. Previously frozen macrophages from three donors were thawed and transduced with 3000 MOI of Ad5f35 vector comprising CTX_269. After 48 hours, the macrophages were placed on plates functionalized with titrations of mesothelin. After 24 hours, supernatants were harvested and analyzed using Meso Scale Discovery QuickPlex SQ 120.

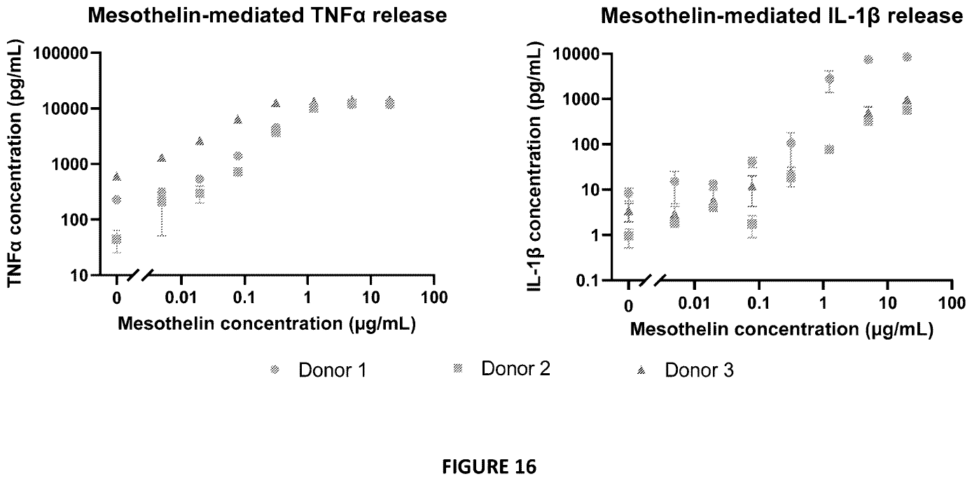

As shown in FIG. 16, macrophages transduced with CTX_269 released cytokines (TNFα and IL-1P) in response to stimulation with mesothelin, and the increase in cytokine release increased with mesothelin concentration.

### Example 9B: Cytokine Release from Macrophages Transduced with an Anti-Mesothelin Binder after Exposure to Cell Lines Expressing Mesothelin

The present Example assesses the cytokine release of macrophages transduced with CTX_269 after contact with cell lines expressing mesothelin. Previously frozen macrophages from two donors were thawed and transduced with 3000 MOI of Ad5f35 vector comprising CTX_269. After 48 hours, the macrophages were plated with cell lines expressing mesothelin in a 1:1 E:T ratio. After 24 hours, supernatants were harvested and analyzed using Meso Scale Discovery QuickPlex SQ 120.

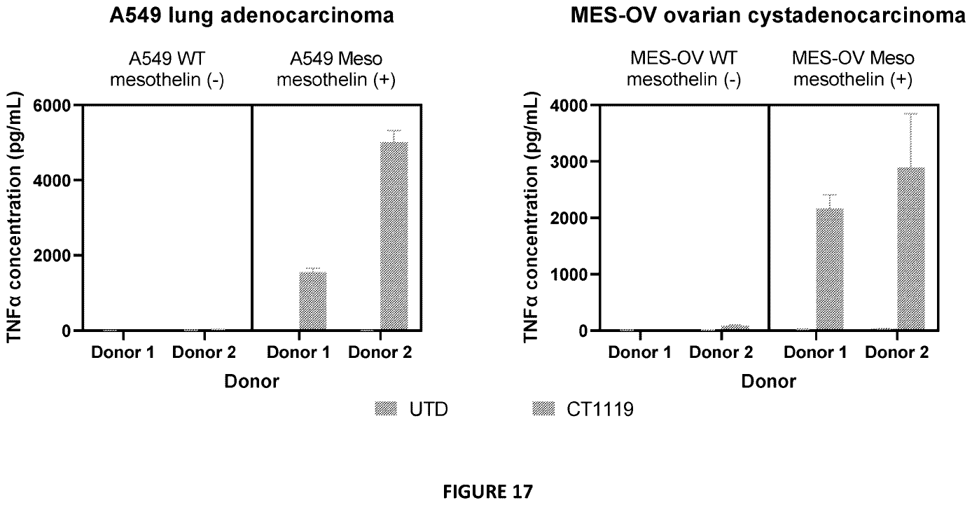

As shown in FIG. 17, macrophages transduced with CTX_269 exhibited cytokine (TNFα) release when target cells (A549 lung adenocarcinoma cells and ovarian cystadenocarcinoma cells) expressed mesothelin.

### Example 10: Effect of Macrophages Transduced with an Anti-Mesothelin Binder in an In Vivo Model

The present Example assesses the effect of macrophages transduced with CTX_269 in an in vivo model. On day 0, mice were challenged with 0.5e6 A549 lung adenocarcinoma cells expressing mesothelin and mKate2/CBG via tail vein injection. On days 5 and 12, previously frozen untransduced (UTD) macrophages from one donor were thawed and transduced with 3000 MOI of Ad5f35 vector comprising CTX_269. On days 7 and 14, mice were treated with the transduced macrophages via tail vein injection, treated with UTD macrophage controls, or not treated (saline injections without macrophages). Tumor burden in the mice was measured via BLI 2-3 times per week. A two-way ANOVA with Geisser-Greenhouse correction and Tukey's multiple comparison correction was performed on tumor burden data collected at the last time point.

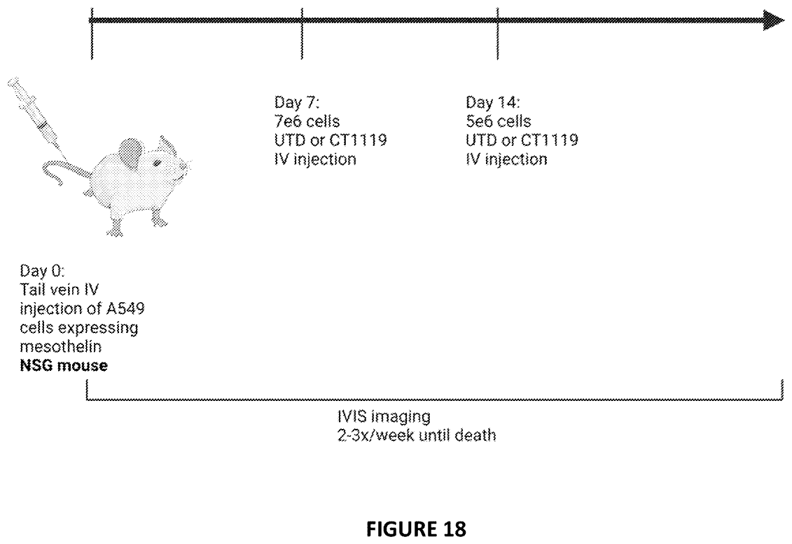

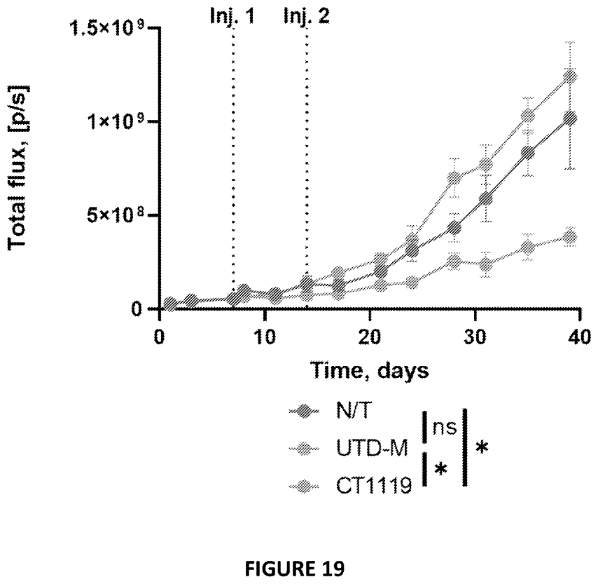

FIG. 18 illustrates the experimental design described above. FIG. 19 shows that a significant reduction in tumor burden was observed in mice treated with macrophages transduced with anti-mesothelin CARs, compared to untreated or UTD macrophage controls.

### Example 11: Expression of a CD28-Based Anti-Mesothelin CAR and Associated Killing of Target Cells

The present Example assesses the expression of an exemplary CD28-based CAR in macrophages. Previously frozen untransduced (UTD) macrophages from two donors were thawed and transduced with lentivirus vector comprising a CD28-based anti-mesothelin CAR at an MOI of 5. On day 7, the macrophages were harvested, tested for expression of anti-mesothelin CAR, and used for a killing assay. Mesothelin/mKate2-expressing target cells were plated at a constant 10,000 cells per well. Fluorescence was tracked on an Incucyte S3 Live Cell Analysis System for 72 hours, with imaging occurring every 4 hours. Killing was determined by calculating the fluorescence at 72 hours divided by the initial fluorescence.

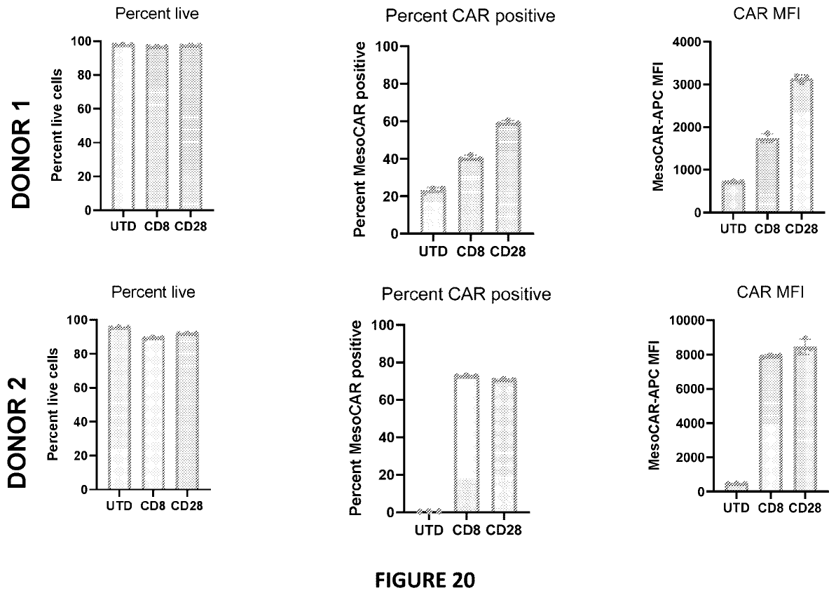

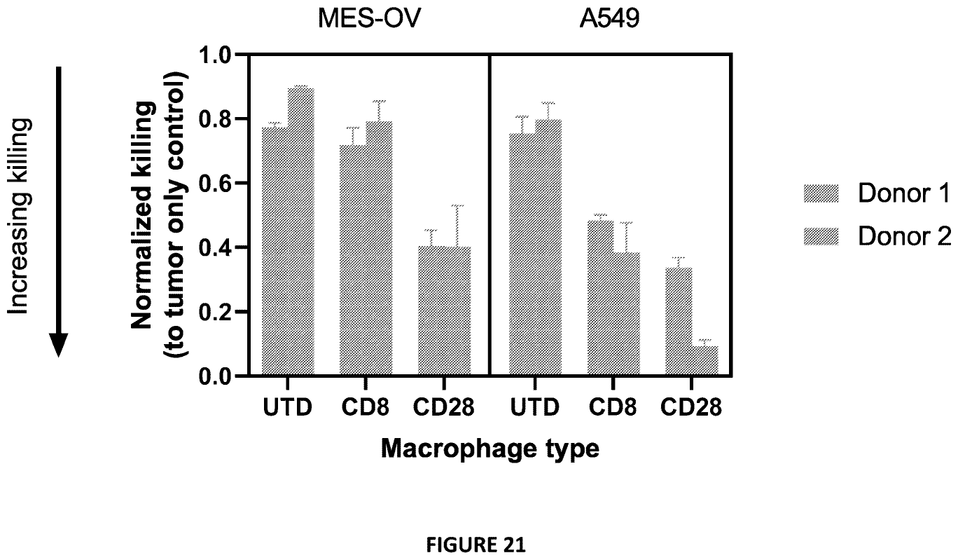

As shown in FIG. 20, the CD28-based anti-mesothelin CAR expresses similarly or better than an exemplary CD8-based anti-mesothelin CAR. Indeed, the CD28-based anti-mesothelin CAR expressed better than the CD8-based anti-mesothelin CAR at the same MOI. Additionally, as shown in FIG. 21, macrophages transduced with the CD28-based anti-mesothelin CAR exhibited enhanced effector function (killing of target cells) compared to macrophages transduced with the CD8-based anti-mesothelin CAR across cell lines. Without wishing to be bound to a theory, the enhanced killing function of the macrophages transduced with the CD28-based anti-mesothelin CAR was likely due to higher levels of CAR expression.

### Example 12: Cytokine Release from Macrophages Transduced with a CD28-Based Anti-Mesothelin CAR after Exposure to Mesothelin

The present Example assesses the cytokine release of macrophages transduced with an exemplary CD28-based anti-mesothelin CAR after exposure to mesothelin. Previously frozen UTD macrophages from two donors were thawed and transduced with lentivirus vector comprising a CD28-based anti-mesothelin CAR at an MOI of 5. On day 4, the macrophages were harvested and plated on titrations of mesothelin. After 24 hours, supernatants were harvested and analyzed using a Meso Scale Discovery QuickPlex SQ 120.

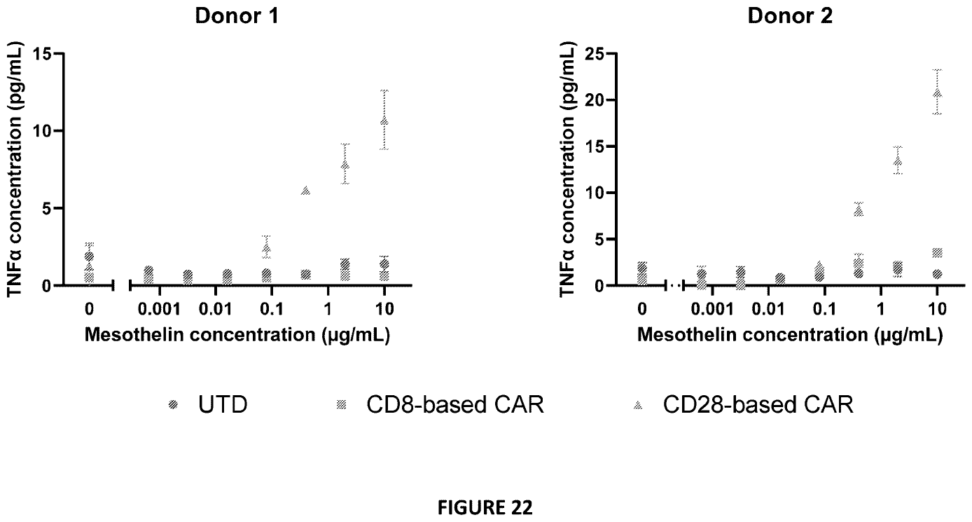

As shown in FIG. 22, macrophages transduced with the CD28-based anti-mesothelin CAR exhibited enhanced cytokine (TNFα) release compared to macrophages transduced with the CD8-based anti-mesothelin CAR.

### Example 13: Resistance of Macrophages Transduced with Anti-Mesothelin Binders to M2 Cytokines

The present Example assesses the resistance of macrophages transduced with CTX_269 or CTX_293 to M2 cytokines. Previously frozen macrophages from three donors were thawed and transduced with 3000 MOI of Ad5f35 vector comprising CTX_269 or CTX_293. After 48 hours, Ad5f35 virus was removed from the macrophage cultures. 72 hours post-transduction, media containing 10 ng/mL IL-10 was added to the macrophages. 24 hours after cytokine (IL-10) addition, macrophages were phenotyped for M/M2 marker expression.

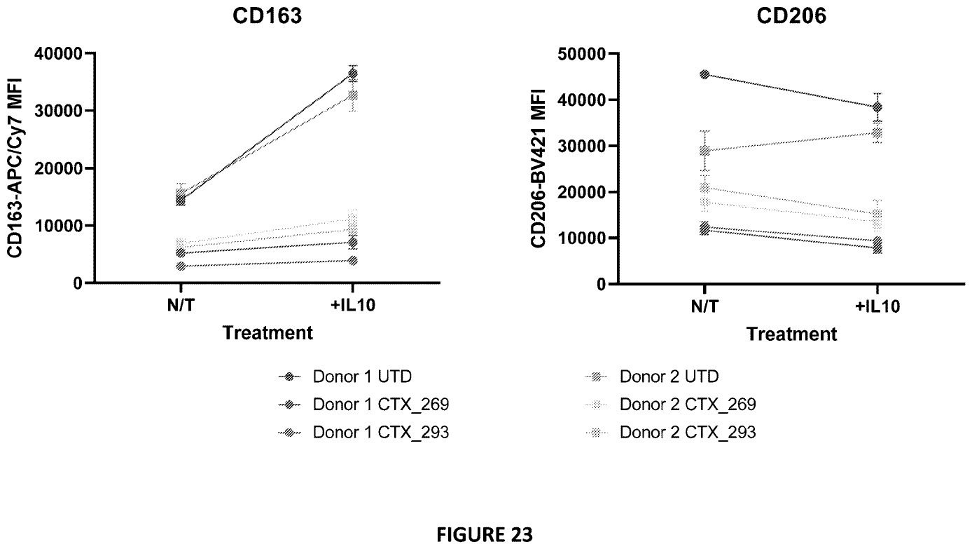

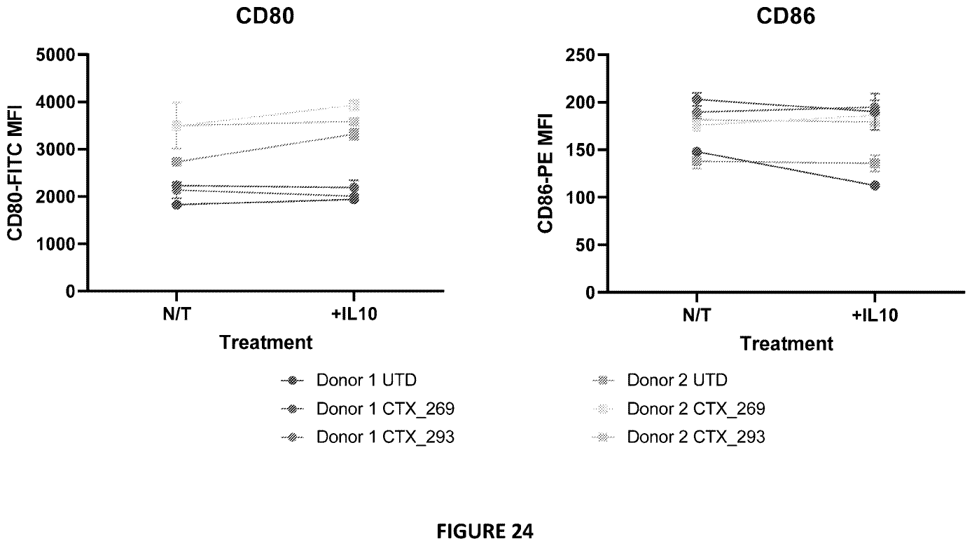

As shown in FIG. 23, macrophages transduced with both CD8-framework (CTX_269) and CD28-framework (CTX_293) CARs exhibited a reduced upregulation of CD163 relative to untransduced (UTD) controls when exposed to IL-10, indicating resistance to polarization to an M2-like phenotype. Similarly, as shown in FIG. 24, macrophages transduced with either CTX_269 or CTX_293 exhibited a reduced downregulation of CD86 relative to untransduced (UTD) controls when exposed to IL-10, suggesting maintenance of the M1 phenotype.

### Example 14: Killing of Tumor Cell Lines with Macrophages Transduced with Anti-Mesothelin Binders

The present Example assesses the killing ability of macrophages transduced with CTX_269. Previously frozen untransduced macrophages from three donors were thawed and transduced with 3000 MOI of Ad5f35 vector comprising CTX_269 or CTX_293. After 48 hours, the macrophages were harvested, tested for expression of anti-mesothelin CAR, and used for a killing assay. Mesothelin/mKate2-expressing target cells were plated at a constant 10,000 cells per well. Fluorescence was tracked on an Incucyte S3 Live Cell Analysis System for 72 hours, with imaging occurring every 4 hours. Killing of target cells was determined by calculating the fluorescence at 72 hours divided by the initial fluorescence.

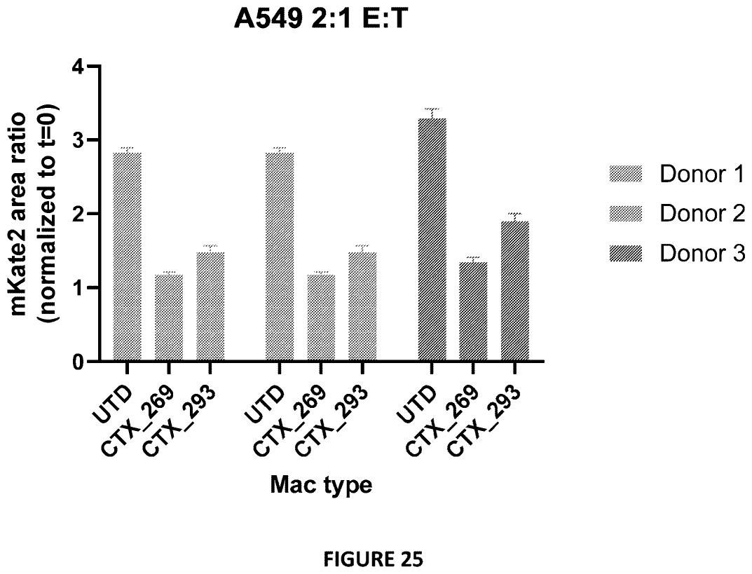

As shown in FIG. 25, macrophages transduced with CTX_293 killed target cells similarly to macrophages transduced with CTX_269.

### Example 15: Effect of Transduction with an Anti-Mesothelin CAR on Monocytes

The present Example assesses the effect of transduction of monocytes with an anti-mesothelin CAR. Previously frozen CD14+ monocytes from two donors were thawed and transduced with 3000 MOI of Ad5f35 vector comprising CTX_001 or CTX_269. Additional monocytes were cultured in GMCSF for differentiation into macrophages. On day 5, untransduced monocytes (now macrophages) were transduced with 3000 MOI of Ad5f35 vector comprising CTX_001 or CTX_269. On day 7, CAR monocytes and macrophages were harvested, phenotyped via flow cytometry, and used in a killing assay. Multiple comparisons-adjusted t tests were performed between the HER2 CAR control data and the mesothelin CAR data.

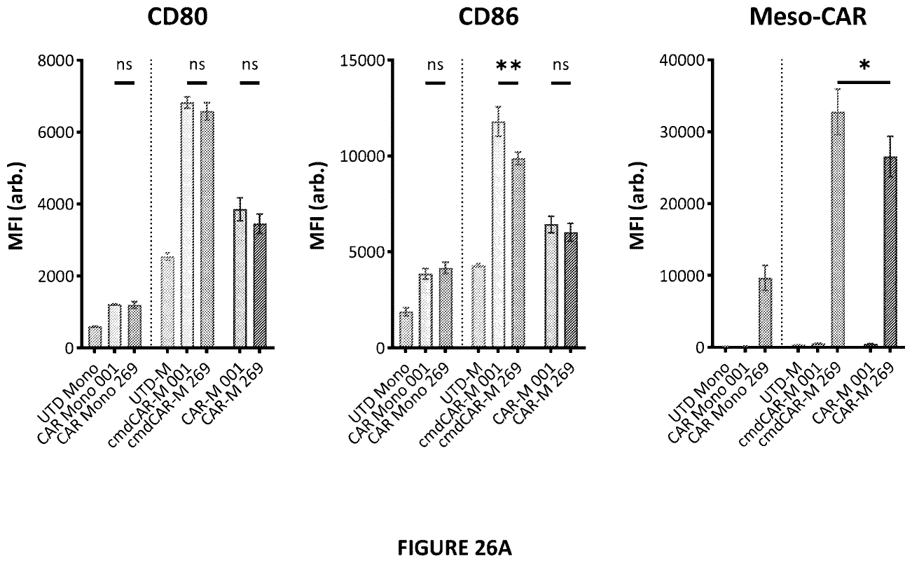

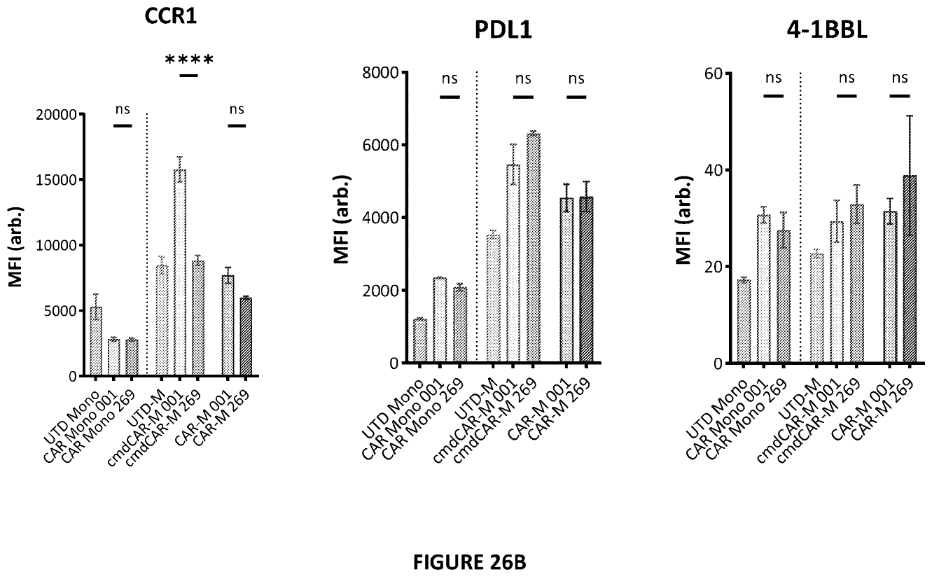

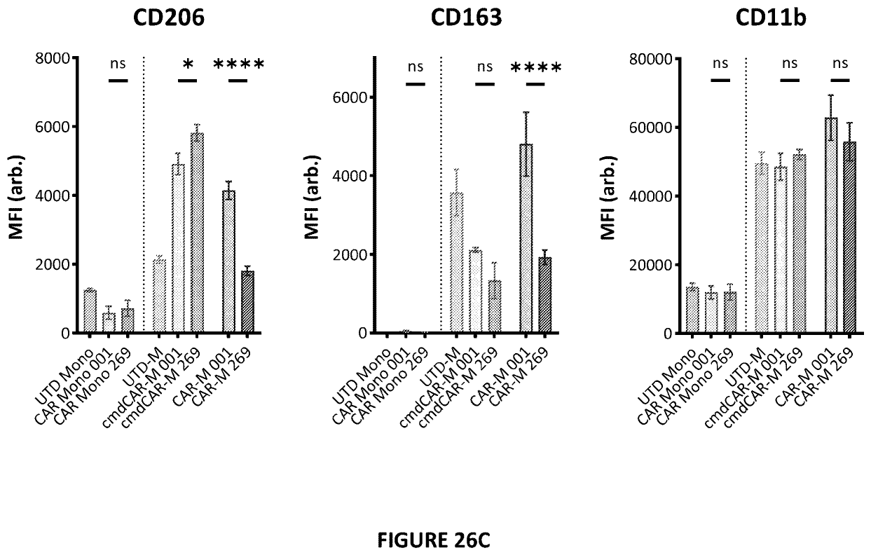

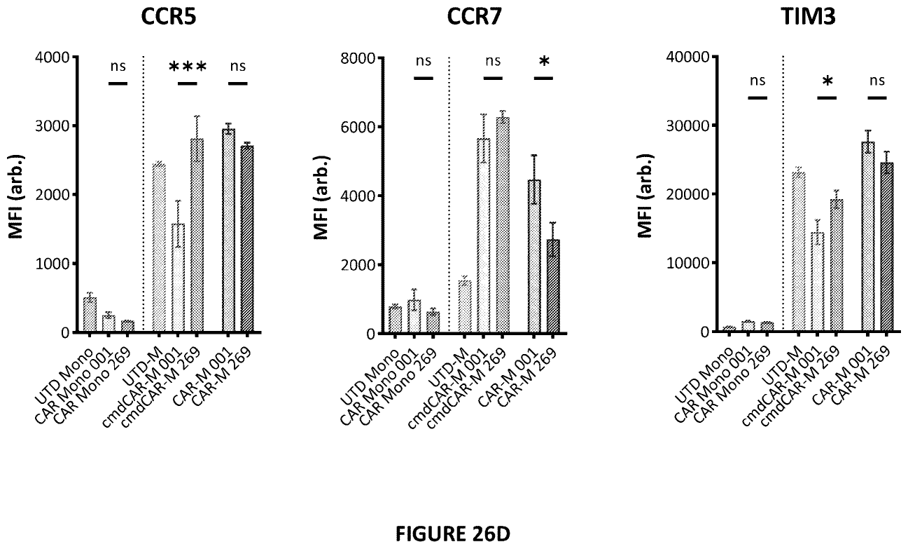

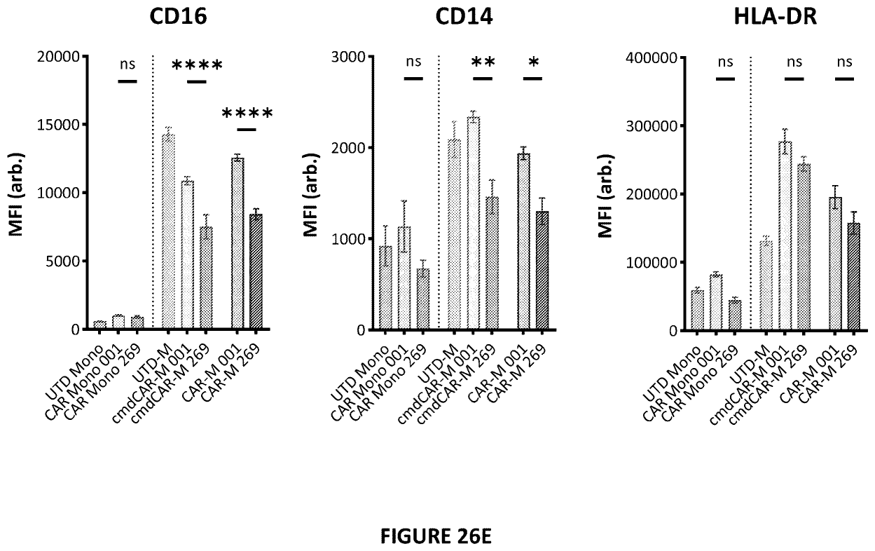

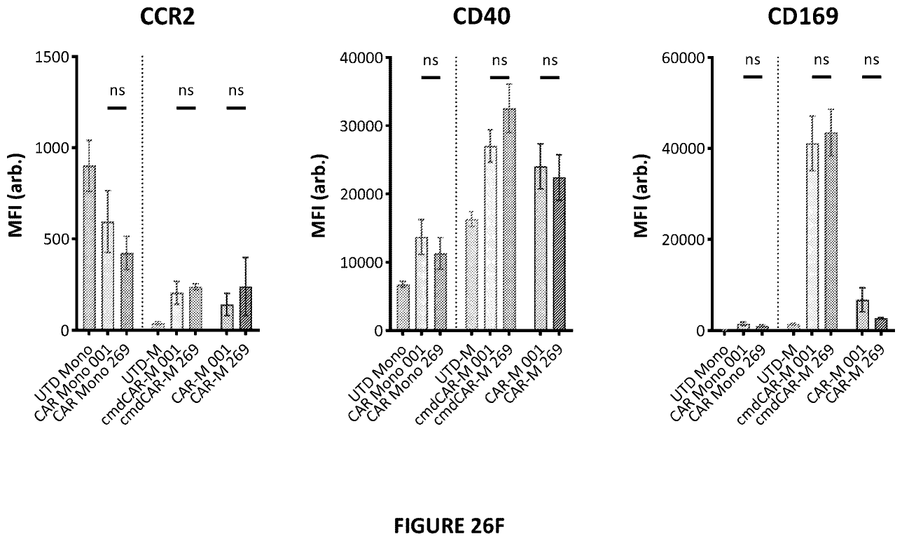

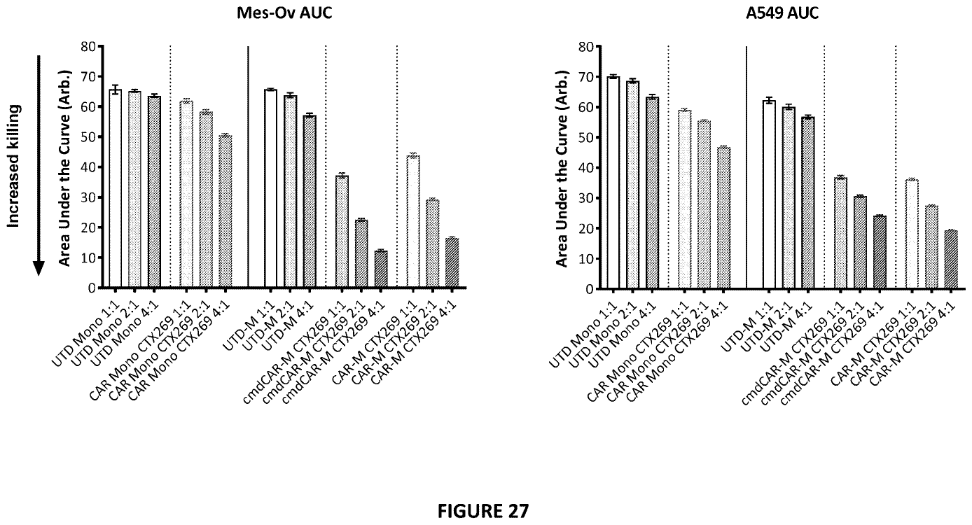

As shown in FIGS. 26A-F, CTX_269 (CD8-based anti-mesothelin CAR) monocytes exhibited a similar phenotype to CTX_001 (anti-HER2 CAR) monocytes. The groups shown in FIGS. 27A-F include: untransduced monocytes (UTD Mono), monocytes transduced with CTX_001 (CAR Mono 001), monocytes transduced with CTX_269 (CAR Mono 269), untransduced monocytes that were differentiated into macrophages (UTD-M), monocytes transduced with CTX_001 and then differentiated into macrophages (cmdCAR-M 001), monocytes transduced with CTX_0269 and then differentiated into macrophages (cmdCAR-M 269), macrophages transduced with CTX_001 after differentiation from monocytes (cmdCAR-M 001), and macrophages transduced with CTX_0269 after differentiation from monocytes (cmdCAR-M 269). Additionally, as shown in FIG. 28, monocytes transduced with a CAR vector and then differentiated into macrophages (cmdCAR-M) and macrophages transduced with a CAR vector after differentiation from monocytes (CAR-M) exhibited similar levels of effector function (killing of target cells).

### Example 16: Effect of Macrophages Transduced with an Anti-Mesothelin Binder in an In Vitro Model

The present Example assesses the phenotype, phagocytosis ability, and cytokine release of macrophages transduced with CTX_269 in an in vitro model. Previously frozen macrophages from two donors were thawed and transduced with 3000 MOI of Ad5f35 vector comprising CTX_269. Cells were maintained in Tex10 (TexMACS+10% FBS+1% pen/strep) for the indicated number of days. For assessment of CAR expression, macrophages from each condition were plated at 50,000 macrophages per well and stained with mesothelin-biotin, followed by staining with streptavidin-APC. Data were acquired using an Attune Cytometer (ThermoFischer). For assessment of cell killing, macrophages from each condition were plated in 100 μL Tex10 and incubated for 30-45 minutes at 37 °C followed by addition of K562 WT or MESO cells for a final volume of 200 L. All tumor cell lines were engineered to express GFP. Plates were incubated at room temperature for 15 minutes after tumor cell addition, followed by 45 minutes at 37 °C. Co-cultures were monitored using an Incucyte, imaging for cell phase and GFP signal for 72 hours. For assessment of cytokine release, macrophages from each condition were co-cultured with K562 cells at an E:T ratio of 1:1. Cells were cultured for 24 hours at 37 °C. 75 μL of supernatant was harvested and immediately measured for cytokines (TNFα) using a MesoScale Discovery System.

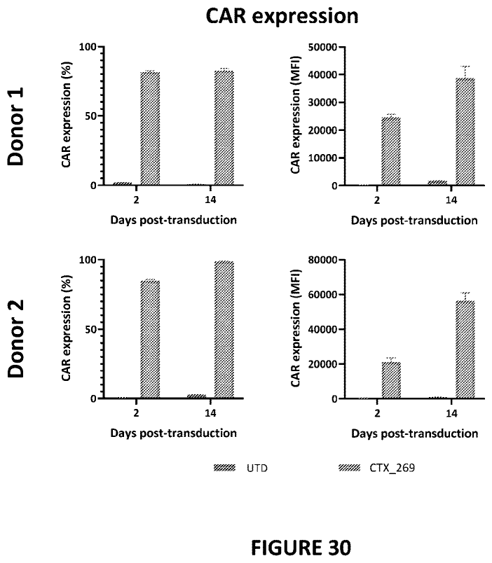

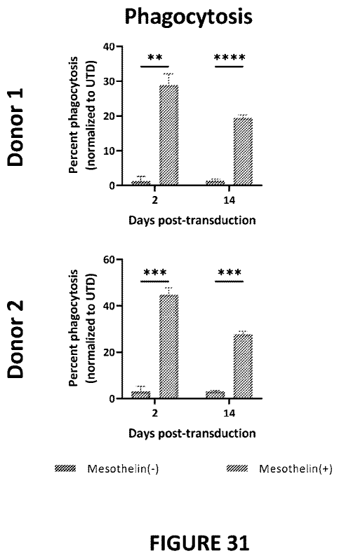

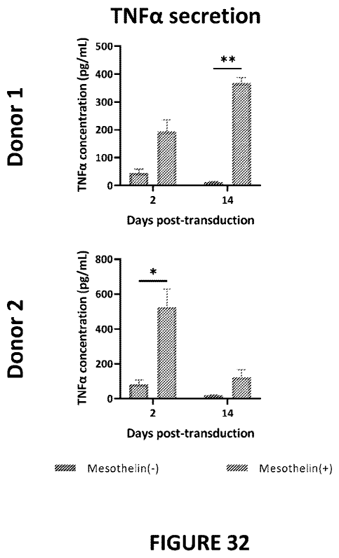

As shown in FIG. 30, anti-mesothelin CAR (CTX_269) expression was robust for at least two weeks. As shown in FIG. 31, CTX_269 transduced macrophages only show increased phagocytosis of mesothelin-positive cells, indicating that phagocytosis of K562 cells by macrophages transduced with CTX_269 involved mesothelin expression by the target cells. The observed increase in mesothelin-specific phagocytosis was present at days 2 and 14, indicating that macrophages transduced with CTX_269 maintain their killing activity for at least two weeks. As shown in FIG. 32, macrophages transduced with CTX_269 exhibited cytokine (TNFα) release when target cells (K562 cells) expressed mesothelin.

### Example 17: Effect of Macrophages Transduced with an Anti-Mesothelin Binder in an In Vivo Model

The present Example assesses the effect of macrophages transduced with CTX_269 in an in vivo model. On day 0, mice were challenged with 0.5e6 A549 lung adenocarcinoma cells expressing mesothelin and mKate2/CBG via tail vein injection. On days 5 and 12, previously frozen untransduced (UTD) macrophages from one donor were thawed and transduced with 3000 MOI of Ad5f35 vector comprising CTX_269. On days 7 and 14, mice were treated with the transduced macrophages via tail vein injection, treated with UTD macrophage controls, or not treated (saline injections without macrophages). Tumor burden in the mice was measured via BLI 2-3 times per week. A two-way ANOVA with Geisser-Greenhouse correction and Tukey's multiple comparison correction was performed on tumor burden data collected at the last time point. On day 50, mice were euthanized and lung tissue was harvested for sectioning and IHC staining (Histowiz) with anti-human mesothelin antibody (Origene, catalog number TA805169BM). Images were analyzed using HALO software for number of nodules per given area (converted to nodules/cm²) and ratio of tumor area to total area of sample.

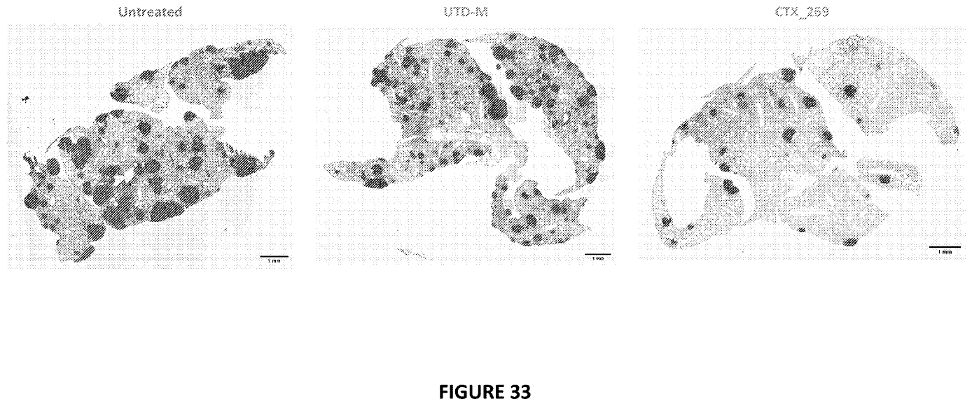

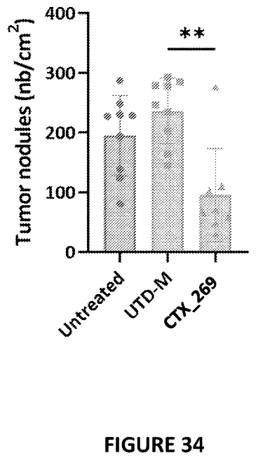

FIG. 18 illustrates the experimental design described above. FIG. 33 shows representative tissue sections of murine lung tumor nodules IHC stained for human mesothelin. FIG. 34 shows that a significant reduction in lung tumor nodules was observed in mice treated with macrophages transduced with anti-mesothelin CARs, compared to untreated or UTD macrophage controls.

### Example 18: Expression of a CD28-Based Anti-Mesothelin CAR and Viability of CAR Monocytes and Macrophages

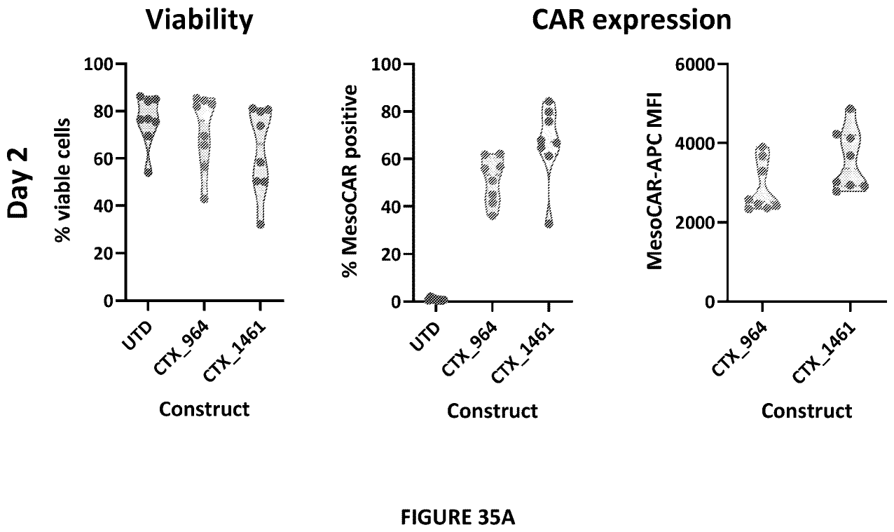

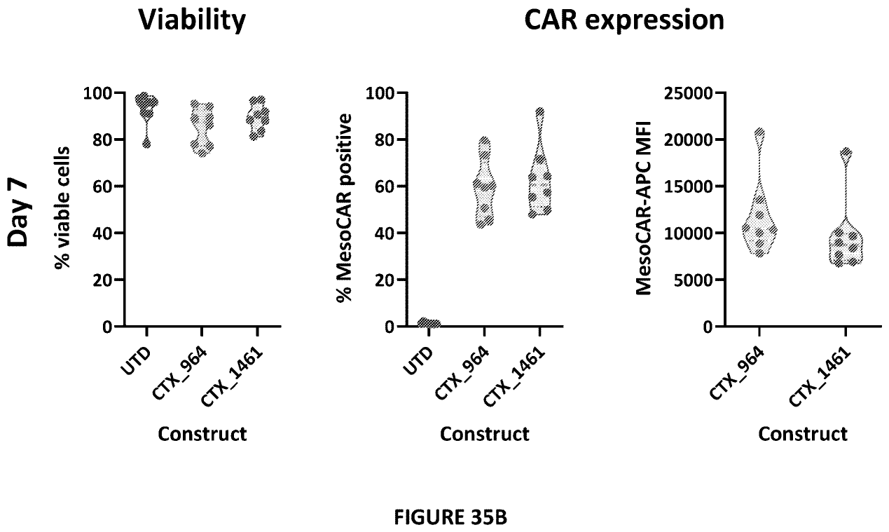

The present Example assesses the expression of an exemplary CD28-based CAR in monocytes and macrophages. Previously frozen CD14+ monocytes from eight donors were transduced with Ad5f35 virus containing either CTX_964 or CTX_1461 at 5 IFU/cell for 1 hour at a concentration of 3e6 cells/mL in Tex10 media containing 10 ng/mL hGM-CSF. Cells were then cultured for two days in order to produce CAR monocytes or seven days in order to produce CAR monocyte-derived CAR macrophages. Viability and CAR expression were assessed via flow cytometry. Each point in FIG. 35A and FIG. 35B represents the average of technical triplicates for each donor.

As shown in FIGS. 35A and 35B, for both Day 2 (monocytes) and Day 7 (monocyte-derived macrophages), cells transduced with Ad5f35 virus containing either CTX_964 or CTX_1461 exhibited viability similar to untransduced control cells. Additionally, CAR expression was similar in cells transduced with Ad5f35 virus containing CTX_964 and in cells transduced with Ad5f35 virus containing CTX_1461.

### Example 19: Comparison of SIRPa Expression in Cells Tranduced with CTX_964 or CTX_1461

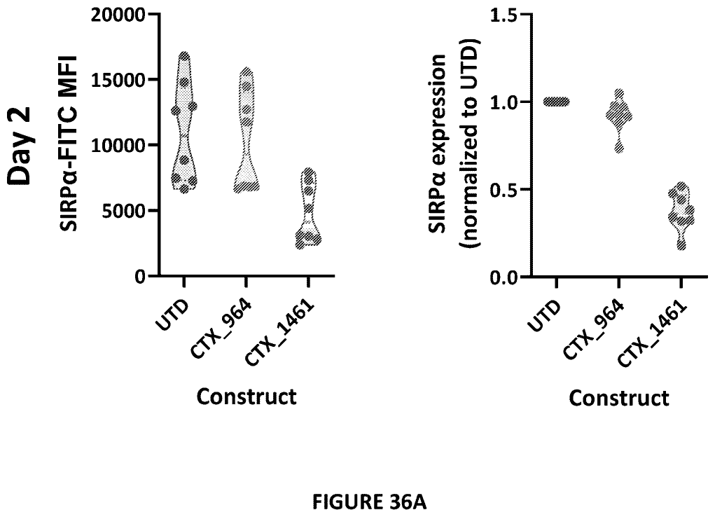

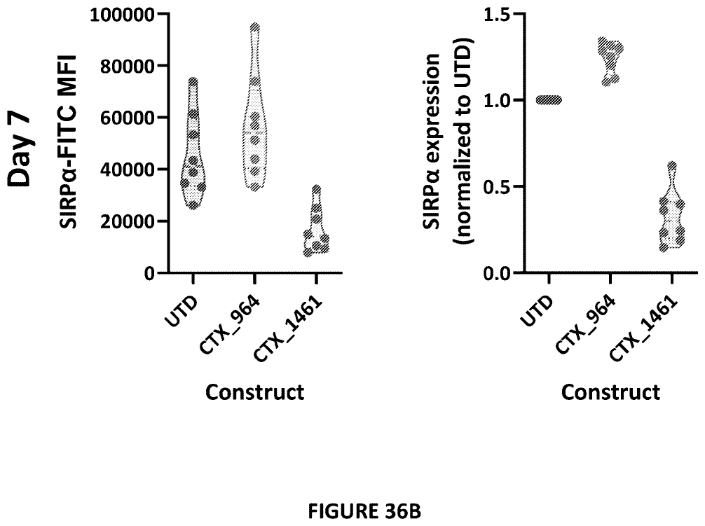

The present Example assesses the expression of SIRPα in monocytes and macrophages expressing exemplary CAR constructs. Previously frozen CD14+ monocytes from eight donors were transduced with Ad5f35 virus containing either CTX_964 or CTX_1461 (CTX964+ intronic shRNA against SIRPα) at 5 IFU/cell for 1 hour at a concentration of 3e6 cells/mL in Tex10 media containing 10 ng/mL hGM-CSF. Cells were then cultured for two days in order to produce CAR monocytes or seven days in order to produce CAR monocyte-derived CAR macrophages. SIRPa expression was measured via flow cytometry. SIRPa expression normalized to untransduced control cells (UTD) was calculated by determining the fold change in fluorescence when using a SIRPa targeting mAb compared to an isotype control, and then normalizing to UTD values. Each point in FIG. 36A and FIG. 36B represents the average of technical triplicates for each donor.

As shown in FIGS. 36A and 36B, for both Day 2 (monocytes) and Day 7 (monocyte-derived macrophages), cells transduced with Ad5f35 virus containing CTX_1461 exhibited reduced SIRPa expression compared to either UTD cells or cells transduced with CTX_964. This illustrates that the intronic shRNA against SIRPa component of CTX_1461 successfully reduces SIRPa expression relative to cells expressing a CAR expressed from CTX_964 (without the intronic shRNA component).

### Example 20: Cytokine Release from Cells Transduced with a CD28-Based Anti-Mesothelin CAR after Exposure to Mesothelin

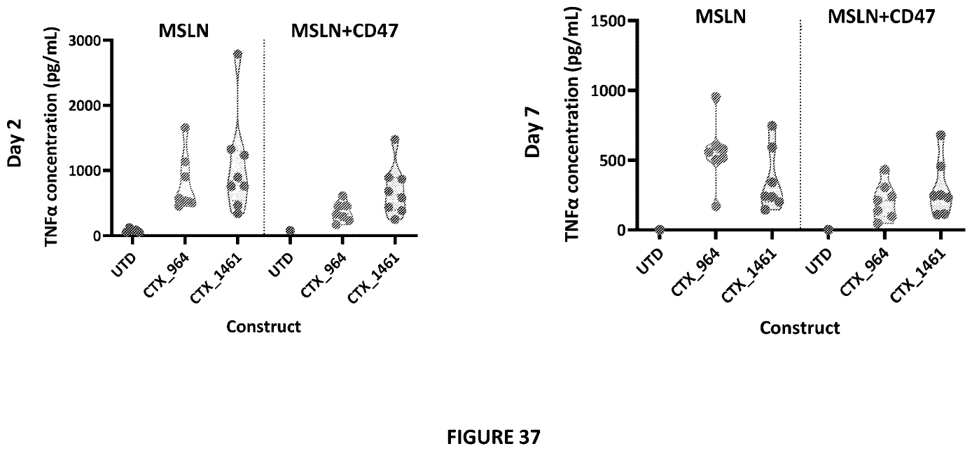

The present Example assesses the cytokine release of cells transduced with an exemplary CD28-based anti-mesothelin CAR after exposure to mesothelin. Previously frozen CD14+ monocytes from eight donors were transduced with Ad5f35 virus containing either CTX_964 or CTX_1461 at 5 IFU/cell for 1 hour at a concentration of 3e6 cells/mL in Tex10 media containing 10 ng/mL hGM-CSF. Cells were then cultured for two days in order to produce CAR monocytes or seven days in order to produce CAR monocyte-derived CAR macrophages. On the appropriate day, cells were harvested and plated at 50e3 viable cells per well in a 96 well plate coated with either 2 μg/mL recombinant human mesothelin or 2 μg/mL recombinant mesothelin+0.5 μg/mL recombinant human CD47. Supernatants were harvested after 24 hours and analyzed via nELISA. Each point in FIG. 37 represents the average of technical triplicates for each donor.

As shown in FIG. 37, monocytes or macrophages transduced with the CTX_964 or CTX_1461 anti-mesothelin CAR constructs exhibited enhanced cytokine (TNFα) release compared to UTD cells, even in the presence of CD47 (MSLN+CD47).

### Example 21: Killing of Tumor Cell Lines with Cells Transduced with Anti-Mesothelin Binders

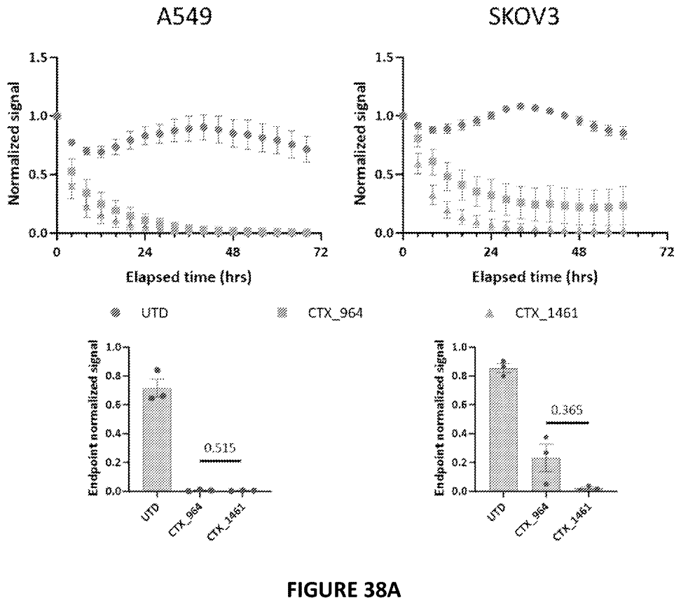

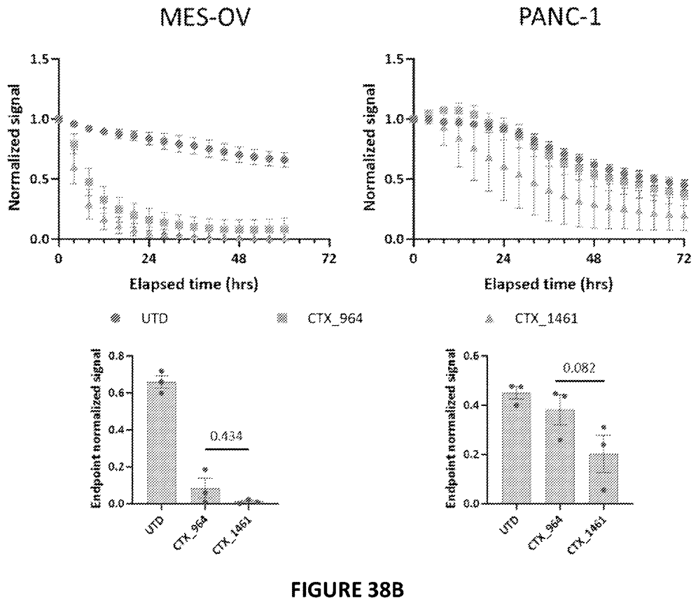

The present Example assesses the killing ability of cells transduced with CTX_964 or CTX_1461. Previously frozen CD14+ monocytes were transduced with Ad5f35 virus containing either CTX_964 or CTX_1461 at 5 IFU/cell for 1 hour at a concentration of 3e6 cells/mL in Tex10 media containing 10 ng/mL hGM-CSF. Cells were then cultured for seven days in order to produce CAR monocyte-derived CAR macrophages. CAR macrophages were plated at 20e3 viable cells per well in a 96 well plate along with target cells expressing fluorescent protein in a 1:1 E:T (effector to target) ratio. Killing over time was monitored by the change in fluorescence normalized to target-only control wells. In the time course plots shown in FIG. 38A and FIG. 38B, each point represents the mean+/−SEM of the three donors, while the bar plots show the individual donors as points.

As shown in FIGS. 38A and 38B, macrophages transduced with either CTX_964 or CTX_1461 were able to kill mesothelin-expressing target cells.

### Example 22: Effect of Cells Transduced with an Anti-Mesothelin Binder in an In Vivo Model

The present Example assesses the effect of cells transduced with CTX_964 or CTX_1461 in an in vivo model. Previously frozen CD14+ monocytes were transduced with Ad5f35 virus containing either CTX_964 or CTX_1461 at 5 IFU/cell for 1 hour at a concentration of 3e6 cells/mL with 10 ng/mL hGM-CSF. Cells were then cultured for seven days in order to produce CAR monocyte-derived CAR macrophages. NSG-S mice were injected with SKOV3-CBG/GFP-CTX1698-Meso cells IP, 1e5 cells/mouse. Mice were imaged with IVIS, randomized based on whole body bioluminescence signal and injected with CAR monocyte-derived CAR macrophages 5e6 cells/mouse within ~3-4 hours after tumor cell injection. Tumor burden was assessed every 3-4 days with IVIS bioluminescence imaging.

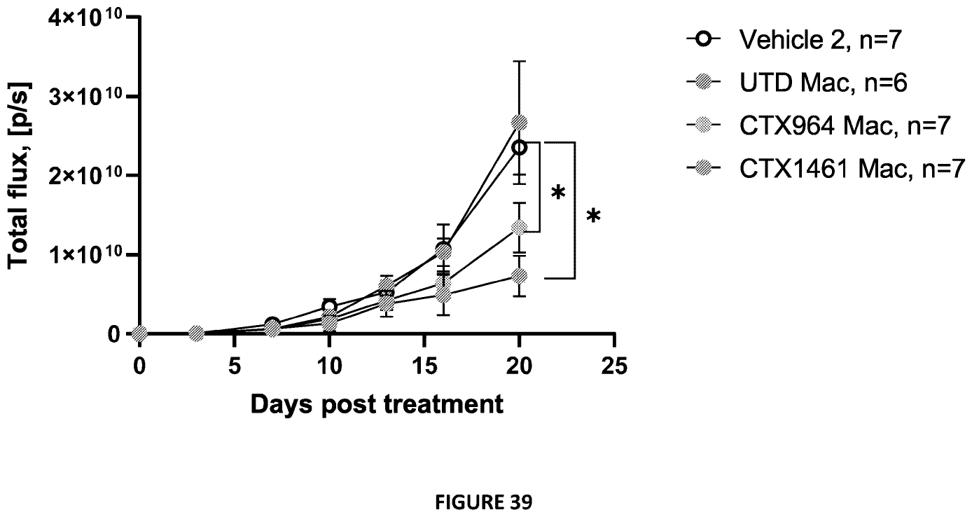

As shown in FIG. 39, CAR monocyte-derived CAR macrophages expressing CTX_964 or CTX_1461 were able to suppress tumor growth in an in vivo xenograft model.

### EXEMPLARY SEQUENCES

TABLE 2
Exemplary Amino Acid Sequences for CARs described herein

| CAR | Amino Acid Sequence |
|---|---|
| CTX_269 | MALPVTALLLPLALLLHAARPGSQVQLVQSGGGLVQPGRSLRLSCAASGF |
| | TFDDYAMHWVRQAPGKGLEWVSGISWNSGSIGYADSVKGRFTISRDNAK |
| | NSLYLQMNSLRAEDTAVYYCAKDGSSSWSWGYFDYWGQGTLVTVSSGG |
| | GGSGGGGSGGGGSSSELTQDPAVSVALGQTVRTTCQGDALRSYYASWYQ |
| | QKPGQAPMLVIYGKNNRPSGIPDRFSGSDSGDTASLTITGAQAEDEADYY |
| | CNSRDSSGYPVFGTGTKVTVLTTTPAPRPPTPAPTIASQPLSLRPEACRPAA |
| | GGAVHTRGLDFACDIYIWAPLAGTCGVLLLSLVITLYCRVKFSRSADAPA |
| | YQQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQA |
| | LPPR (SEQ ID NO: 1) |
| CTX_293 | MALPVTALLLPLALLLHAARPGSQVQLVQSGGGLVQPGRSLRLSCAASGF |
| | TFDDYAMHWVRQAPGKGLEWVSGISWNSGSIGYADSVKGRFTISRDNAK |
| | NSLYLQMNSLRAEDTAVYYCAKDGSSSWSWGYFDYWGQGTLVTVSSGG |
| | GGSGGGGGGGGSSSELTQDPAVSVALGQTVRTTCQGDALRSYYASWYQ |
| | QKPGQAPMLVIYGKNNRPSGIPDRFSGSDSGDTASLTITGAQAEDEADYY |
| | CNSRDSSGYPVFGTGTKVTVLIEVMYPPPYLDNEKSNGTIIHVKGKHLCPS |
| | PLFPGPSKPFWVLVVVGGVLACYSLLVTVAFIIFWVRVKFSRSADAPAYQ |
| | QGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALP |
| | PR (SEQ ID NO: 2) |
| CTX_964 | MALPVTALLLPLALLLHAARPGSQVQLVQSGGGLVQPGRSLRLSCAASGF |
| | TFDDYAMHWVRQAPGKGLEWVSGISWNSGSIGYADSVKGRFTISRDNAK |
| | NSLYLQMNSLRAEDTAVYYCAKDGSSSWSWGYFDYWGQGTLVTVSSGG |
| | GGSGGGGGGGGSSSELTQDPAVSVALGQTVRTTCQGDALRSYYASWYQ |
| | QKPGQAPMLVIYGKNNRPSGIPDRFSGSDSGDTASLTITGAQAEDEADYY |
| | CNSRDSSGYPVFGTGTKVTVLIEVMYPPPYLDNEKSNGTIIHVKGKHLCPS |
| | PLFPGPSKPFWVLVVVGGVLACYSLLVTVAFIIFWVRLKIQVRKAAITSYE |
| | KSDGVYTGLSTRNQETYETLKHEKPPQ (SEQ ID NO: 3) |
| CTX_760 | MALPVTALLLPLALLLHAARPQVQLVQSGGGLVQPGRSLRLSCAASGFTF |
| | DDYAMHWVRQAPGKGLEWVSGISWNSGSIGYADSVKGRFTISRDNAKNS |
| | LYLQMNSLRAEDTAVYYCAKDGSSSWSWGYFDYWGQGTLVTVSSGGGG |
| | SGGGGSGGGGSSSELTQDPAVSVALGQTVRTTCQGDALRSYYASWYQQK |
| | PGQAPMLVIYGKNNRPSGIPDRFSGSDSGDTASLTITGAQAEDEADYYCNS |
| | RDSSGYPVFGTGTKVTVLTTTPAPRPPTPAPTIASQPLSLRPEACRPAAGGA |
| | VHTRGLDFACDIYIWAPLAGTCGVLLLSLVITLYCRVKFSRSADAPAYQQ |
| | GQNQLYNELNLGRREEYDVLDKRRGRDPEMGGKPRRKNPQEGLYNELQ |
| | KDKMAEAYSEIGMKGERRRGKGHDGLYQGLSTATKDTYDALHMQALPP |
| | RRAKRSGSGATNFSLLKQAGDVEENPGPMIETYNQTSPRSAATGLPISMKI |
| | FMYLLTVFLITQMIGSALFAVYLHRRLDKIEDERNLHEDFVFMKTIQRCNT |
| | GERSLSLLNCEEIKSQFEGFVKDIMLNKEETKKENSFEMQKGDQNPQIAAH |
| | VISEASSKTTSVLQWAEKGYYTMSNNLVTLENGKQLTVKRQGLYYIYAQ |
| | VTFCSNREASSQAPFIASLCLKSPGRFERILLRAANTHSSAKPCGQQSIHLG |
| | GVFELQPGASVFVNVTDPSQVSHGTGFTSFGLLKL (SEQ ID NO: 4) |
| CTX_761 | MALPVTALLLPLALLLHAARPQVQLVQSGGGLVQPGRSLRLSCAASGFTF |
| | DDYAMHWVRQAPGKGLEWVSGISWNSGSIGYADSVKGRFTISRDNAKNS |
| | LYLQMNSLRAEDTAVYYCAKDGSSSWSWGYFDYWGQGTLVTVSSGGGG |
| | SGGGGSGGGGSSSELTQDPAVSVALGQTVRTTCQGDALRSYYASWYQQK |
| | PGQAPMLVIYGKNNRPSGIPDRFSGSDSGDTASLTITGAQAEDEADYYCNS |
| | RDSSGYPVFGTGTKVTVLIEVMYPPPYLDNEKSNGTIIHVKGKHLCPSPLFP |
| | GPSKPFWVLVVVGGVLACYSLLVTVAFIIFWVRVKFSRSADAPAYQQGQ |
| | NQLYNELNLGRREEYDVLDKRRGRDPEMGGKPRRKNPQEGLYNELQKD |
| | KMAEAYSEIGMKGERRRGKGHDGLYQGLSTATKDTYDALHMQALPPRR |
| | AKRSGSGATNFSLLKQAGDVEENPGPMIETYNQTSPRSAATGLPISMKIFM |
| | YLLTVFLITQMIGSALFAVYLHRRLDKIEDERNLHEDFVFMKTIQRCNTGE |
| | RSLSLLNCEEIKSQFEGFVKDIMLNKEETKKENSFEMQKGDQNPQIAAHVI |
| | SEASSKTTSVLQWAEKGYYTMSNNLVTLENGKQLTVKRQGLYYIYAQVT |
| | FCSNREASSQAPFIASLCLKSPGRFERILLRAANTHSSAKPCGQQSIHLGGV |
| | FELQPGASVFVNVTDPSQVSHGTGFTSFGLLKL (SEQ ID NO: 5) |
| CTX_762 | MALPVTALLLPLALLLHAARPQVQLVQSGGGLVQPGRSLRLSCAASGFTF |
| | DDYAMHWVRQAPGKGLEWVSGISWNSGSIGYADSVKGRFTISRDNAKNS |
| | LYLQMNSLRAEDTAVYYCAKDGSSSWSWGYFDYWGQGTLVTVSSGGGG |
| | SGGGGSGGGGSSSELTQDPAVSVALGQTVRTTCQGDALRSYYASWYQQK |
| | PGQAPMLVIYGKNNRPSGIPDRFSGSDSGDTASLTITGAQAEDEADYYCNS |
| | RDSSGYPVFGTGTKVTVLIEVMYPPPYLDNEKSNGTIIHVKGKHLCPSPLFP |
| | GPSKPFWVLVVVGGVLACYSLLVTVAFIIFWVMAAGGPGAGSAAPVSSTS |
| | SLPLAALNMRVRRRLSLFLNVRTQVAADWTALAEEMDFEYLEIRQLETQA |
| | DPTGRLLDAWQGRPGASVGRLLELLTKLGRDDVLLELGPSIEEDCQKYIL |
| | KQQQEEAEKPLQVAAVDSSVPRTAELAGITTLDDPLGHMPERFDAFICYCP |
| | SDIRVKFSRSADAPAYQQGQNQLYNELNLGRREEYDVLDKRRGRDPEMG |
| | GKPRRKNPQEGLYNELQKDKMAEAYSEIGMKGERRRGKGHDGLYQGLS |
| | TATKDTYDALHMQALPPR (SEQ ID NO: 6) |
| CTX_763 | MALPVTALLLPLALLLHAARPQVQLVQSGGGLVQPGRSLRLSCAASGFTF |
| | DDYAMHWVRQAPGKGLEWVSGISWNSGSIGYADSVKGRFTISRDNAKNS |
| | LYLQMNSLRAEDTAVYYCAKDGSSSWSWGYFDYWGQGTLVTVSSGGGG |
| | SGGGGSGGGGSSSELTQDPAVSVALGQTVRTTCQGDALRSYYASWYQQK |
| | PGQAPMLVIYGKNNRPSGIPDRFSGSDSGDTASLTITGAQAEDEADYYCNS |
| | RDSSGYPVFGTGTKVTVLIEVMYPPPYLDNEKSNGTIIHVKGKHLCPSPLFP |
| | GPSKPFWVLVVVGGVLACYSLLVTVAFIIFWVMAAGGPGAGSAAPVSSTS |
| | SLPLAALNMRVRRRLSLFLNVRTQVAADWTALAEEMDFEYLEIRQLETQA |
| | DPTGRLLDAWQGRPGASVGRLLELLTKLGRDDVLLELGPSIEEDCQKYIL |
| | KQQQEEAEKPLQVAAVDSSVPRTAELAGITTLDDPLGHMPERFDAFICYCP |
| | SDIKKVAKKPTNKAPHPKQEPQEINFPDDLPGSNTAAPVQETLHGCQPVTQ |
| | EDGKESRISVQERQRVKFSRSADAPAYQQGQNQLYNELNLGRREEYDVL |
| | DKRRGRDPEMGGKPRRKNPQEGLYNELQKDKMAEAYSEIGMKGERRRG |
| | KGHDGLYQGLSTATKDTYDALHMQALPPR (SEQ ID NO: 7) |
| CTX_764 | MALPVTALLLPLALLLHAARPQVQLVQSGGGLVQPGRSLRLSCAASGFTF |
| | DDYAMHWVRQAPGKGLEWVSGISWNSGSIGYADSVKGRFTISRDNAKNS |
| | LYLQMNSLRAEDTAVYYCAKDGSSSWSWGYFDYWGQGTLVTVSSGGGG |
| | SGGGGSGGGGSSSELTQDPAVSVALGQTVRTTCQGDALRSYYASWYQQK |
| | PGQAPMLVIYGKNNRPSGIPDRFSGSDSGDTASLTITGAQAEDEADYYCNS |
| | RDSSGYPVFGTGTKVTVLTTTPAPRPPTPAPTIASQPLSLRPEACRPAAGGA |
| | VHTRGLDFACDIYIWAPLAGTCGVLLLSLVITLYCMAAGGPGAGSAAPVS |
| | STSSLPLAALNMRVRRRLSLFLNVRTQVAADWTALAEEMDFEYLEIRQLE |
| | TQADPTGRLLDAWQGRPGASVGRLLELLTKLGRDDVLLELGPSIEEDCQK |
| | YILKQQQEEAEKPLQVAAVDSSVPRTAELAGITTLDDPLGHMPERFDAFIC |
| | YCPSDIKKVAKKPTNKAPHPKQEPQEINFPDDLPGSNTAAPVQETLHGCQP |
| | VTQEDGKESRISVQERQRVKFSRSADAPAYQQGQNQLYNELNLGRREEY |
| | DVLDKRRGRDPEMGGKPRRKNPQEGLYNELQKDKMAEAYSEIGMKGER |
| | RRGKGHDGLYQGLSTATKDTYDALHMQALPPR (SEQ ID NO: 8) |
| CTX_271 | MALPVTALLLPLALLLHAARPGSEVQLVESGGGLVQPGRSLRLSCAASGF |
| | TFDDYAMHWVRQAPGKGLEWVSGISWNSGSTGYADSVKGRFTISRDNAK |
| | NSLYLQMNSLRAEDTALYYCAKDSSSWYGGGSAFDIWGQGTMVTVSSG |
| | GGGSGGGGSGGGGSSSELTQDPAVSVALGQTVRITCQGDSLRSYYASWY |
| | QQKPGQAPVLVIYGKNNRPSGIPDRFSGSSSGNTASLTITGAQAEDEADYY |
| | CNSRGSSGNHYVFGTGTKVTVLTTTPAPRPPTPAPTIASQPLSLRPEACRPA |
| | AGGAVHTRGLDFACDIYIWAPLAGTCGVLLLSLVITLYCRVKFSRSADAP |
| | AYQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHMQ |
| | ALPPR (SEQ ID NO: 9) |
| CTX_1120 | MALPVTALLLPLALLLHAARPGSQVQLVQSGGGLVQPGRSLRLSCAASGF |
| | TFDDYAMHWVRQAPGKGLEWVSGISWNSGSIGYADSVKGRFTISRDNAK |
| | NSLYLQMNSLRAEDTAVYYCAKDGSSSWSWGYFDYWGQGTLVTVSSGG |
| | GGSGGGGSGGGGSSSELTQDPAVSVALGQTVRTTCQGDALRSYYASWYQ |
| | QKPGQAPMLVIYGKNNRPSGIPDRFSGSDSGDTASLTITGAQAEDEADYY |
| | CNSRDSSGYPVFGTGTKVTVLIEVMYPPPYLDNEKSNGTIIHVKGKHLCPS |
| | PLFPGPSKPFWVLVVVGGVLACYSLLVTVAFIIFWVMAAGGPGAGSAAPV |
| | SSTSSLPLAALNMRVRRRLSLFLNVRTQVAADWTALAEEMDFEYLEIRQL |
| | ETQADPTGRLLDAWQGRPGASVGRLLELLTKLGRDDVLLELGPSIEEDCQ |
| | KYILKQQQEEAEKPLQVAAVDSSVPRTAELAGITTLDDPLGHMPERFDAFI |
| | CYCPSDIKKVAKKPTNKAPHPKQEPQEINFPDDLPGSNTAAPVQETLHGCQ |
| | PVTQEDGKESRISVQERQRLKIQVRKAAITSYEKSDGVYTGLSTRNQETYE |
| | TLKHEKPPQ (SEQ ID NO: 10) |
| CTX_1114 | MALPVTALLLPLALLLHAARPEVQLVESGGGLVQPGRSLRLSCAASGFTF |
| | DDYAMHWVRQAPGKGLEWVSGISWNSGSTGYADSVKGRFTISRDNAKN |
| | SLYLQMNSLRAEDTALYYCAKDSSSWYGGGSAFDIWGQGTMVTVSSGG |
| | GGSGGGGSGGGGSSSELTQDPAVSVALGQTVRITCQGDSLRSYYASWYQ |
| | QKPGQAPVLVIYGKNNRPSGIPDRFSGSSSGNTASLTITGAQAEDEADYYC |
| | NSRGSSGNHYVFGTGTKVTVLIEVMYPPPYLDNEKSNGTIIHVKGKHLCPS |
| | PLFPGPSKPFWVLVVVGGVLACYSLLVTVAFIIFWVRVKFSRSADAPAYQ |
| | QGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALP |
| | PR (SEQ ID NO: 11) |
| CTX_1115 | MALPVTALLLPLALLLHAARPEVQLVESGGGLVQPGRSLRLSCAASGFTF |
| | DDYAMHWVRQAPGKGLEWVSGISWNSGSTGYADSVKGRFTISRDNAKN |
| | SLYLQMNSLRAEDTALYYCAKDSSSWYGGGSAFDIWGQGTMVTVSSGG |
| | GGSGGGGSGGGGSSSELTQDPAVSVALGQTVRITCQGDSLRSYYASWYQ |
| | QKPGQAPVLVIYGKNNRPSGIPDRFSGSSSGNTASLTITGAQAEDEADYYC |
| | NSRGSSGNHYVFGTGTKVTVLIEVMYPPPYLDNEKSNGTIIHVKGKHLCPS |
| | PLFPGPSKPFWVLVVVGGVLACYSLLVTVAFIIFWVRVKFSRSADAPAYQ |
| | QGQNQLYNELNLGRREEYDVLDKRRGRDPEMGGKPRRKNPQEGLYNEL |
| | QKDKMAEAYSEIGMKGERRRGKGHDGLYQGLSTATKDTYDALHMQALP |
| | PRRAKRSGSGATNFSLLKQAGDVEENPGPMIETYNQTSPRSAATGLPISMK |
| | IFMYLLTVFLITQMIGSALFAVYLHRRLDKIEDERNLHEDFVFMKTIQRCN |
| | TGERSLSLLNCEEIKSQFEGFVKDIMLNKEETKKENSFEMQKGDQNPQIAA |
| | HVISEASSKTTSVLQWAEKGYYTMSNNLVTLENGKQLTVKRQGLYYIYA |
| | QVTFCSNREASSQAPFIASLCLKSPGRFERILLRAANTHSSAKPCGQQSIHL |
| | GGVFELQPGASVFVNVTDPSQVSHGTGFTSFGLLKL (SEQ ID NO: 12) |
| CTX_1116 | MALPVTALLLPLALLLHAARPEVQLVESGGGLVQPGRSLRLSCAASGFTF |
| | DDYAMHWVRQAPGKGLEWVSGISWNSGSTGYADSVKGRFTISRDNAKN |
| | SLYLQMNSLRAEDTALYYCAKDSSSWYGGGSAFDIWGQGTMVTVSSGG |
| | GGSGGGGSGGGGSSSELTQDPAVSVALGQTVRITCQGDSLRSYYASWYQ |
| | QKPGQAPVLVIYGKNNRPSGIPDRFSGSSSGNTASLTITGAQAEDEADYYC |
| | NSRGSSGNHYVFGTGTKVTVLIEVMYPPPYLDNEKSNGTIIHVKGKHLCPS |
| | PLFPGPSKPFWVLVVVGGVLACYSLLVTVAFIIFWVMAAGGPGAGSAAPV |
| | SSTSSLPLAALNMRVRRRLSLFLNVRTQVAADWTALAEEMDFEYLEIRQL |
| | ETQADPTGRLLDAWQGRPGASVGRLLELLTKLGRDDVLLELGPSIEEDCQ |
| | KYILKQQQEEAEKPLQVAAVDSSVPRTAELAGITTLDDPLGHMPERFDAFI |
| | CYCPSDIRVKFSRSADAPAYQQGQNQLYNELNLGRREEYDVLDKRRGRD |
| | PEMGGKPRRKNPQEGLYNELQKDKMAEAYSEIGMKGERRRGKGHDGLY |
| | QGLSTATKDTYDALHMQALPPR (SEQ ID NO: 13) |
| CTX_1117 | MALPVTALLLPLALLLHAARPEVQLVESGGGLVQPGRSLRLSCAASGFTF |
| | DDYAMHWVRQAPGKGLEWVSGISWNSGSTGYADSVKGRFTISRDNAKN |
| | SLYLQMNSLRAEDTALYYCAKDSSSWYGGGSAFDIWGQGTMVTVSSGG |
| | GGSGGGGSGGGGSSSELTQDPAVSVALGQTVRITCQGDSLRSYYASWYQ |
| | QKPGQAPVLVIYGKNNRPSGIPDRFSGSSSGNTASLTITGAQAEDEADYYC |
| | NSRGSSGNHYVFGTGTKVTVLIEVMYPPPYLDNEKSNGTIIHVKGKHLCPS |
| | PLFPGPSKPFWVLVVVGGVLACYSLLVTVAFIIFWVMAAGGPGAGSAAPV |
| | SSTSSLPLAALNMRVRRRLSLFLNVRTQVAADWTALAEEMDFEYLEIRQL |
| | ETQADPTGRLLDAWQGRPGASVGRLLELLTKLGRDDVLLELGPSIEEDCQ |
| | KYILKQQQEEAEKPLQVAAVDSSVPRTAELAGITTLDDPLGHMPERFDAFI |
| | CYCPSDIKKVAKKPTNKAPHPKQEPQEINFPDDLPGSNTAAPVQETLHGCQ |
| | PVTQEDGKESRISVQERQRVKFSRSADAPAYQQGQNQLYNELNLGRREEY |
| | DVLDKRRGRDPEMGGKPRRKNPQEGLYNELQKDKMAEAYSEIGMKGER |
| | RRGKGHDGLYQGLSTATKDTYDALHMQALPPR (SEQ ID NO: 14) |
| CTX_1118 | MALPVTALLLPLALLLHAARPEVQLVESGGGLVQPGRSLRLSCAASGFTF |
| | DDYAMHWVRQAPGKGLEWVSGISWNSGSTGYADSVKGRFTISRDNAKN |
| | SLYLQMNSLRAEDTALYYCAKDSSSWYGGGSAFDIWGQGTMVTVSSGG |
| | GGSGGGGSGGGGSSSELTQDPAVSVALGQTVRITCQGDSLRSYYASWYQ |
| | QKPGQAPVLVIYGKNNRPSGIPDRFSGSSSGNTASLTITGAQAEDEADYYC |
| | NSRGSSGNHYVFGTGTKVTVLIEVMYPPPYLDNEKSNGTIIHVKGKHLCPS |
| | PLFPGPSKPFWVLVVVGGVLACYSLLVTVAFIIFWVMAAGGPGAGSAAPV |
| | SSTSSLPLAALNMRVRRRLSLFLNVRTQVAADWTALAEEMDFEYLEIRQL |
| | ETQADPTGRLLDAWQGRPGASVGRLLELLTKLGRDDVLLELGPSIEEDCQ |
| | KYILKQQQEEAEKPLQVAAVDSSVPRTAELAGITTLDDPLGHMPERFDAFI |
| | CYCPSDIKKVAKKPTNKAPHPKQEPQEINFPDDLPGSNTAAPVQETLHGCQ |
| | PVTQEDGKESRISVQERQRVKFSRSADAPAYQQGQNQLYNELNLGRREEY |
| | DVLDKRRGRDPEMGGKPRRKNPQEGLYNELQKDKMAEAYSEIGMKGER |
| | RRGKGHDGLYQGLSTATKDTYDALHMQALPPR (SEQ ID NO: 15) |
| CTX_119 | MALPVTALLLPLALLLHAARPEVQLVESGGGLVQPGRSLRLSCAASGFTF |
| | DDYAMHWVRQAPGKGLEWVSGISWNSGSTGYADSVKGRFTISRDNAKN |
| | SLYLQMNSLRAEDTALYYCAKDSSSWYGGGSAFDIWGQGTMVTVSSGG |
| | GGSGGGGSGGGGSSSELTQDPAVSVALGQTVRITCQGDSLRSYYASWYQ |
| | QKPGQAPVLVIYGKNNRPSGIPDRFSGSSSGNTASLTITGAQAEDEADYYC |
| | NSRGSSGNHYVFGTGTKVTVLIEVMYPPPYLDNEKSNGTIIHVKGKHLCPS |
| | PLFPGPSKPFWVLVVVGGVLACYSLLVTVAFIIFWVRLKIQVRKAAITSYE |
| | KSDGVYTGLSTRNQETYETLKHEKPPQ (SEQ ID NO: 16) |
| CTX_1461 | MALPVTALLLPLALLLHAARPGSQVQLVQSGGGLVQPGRSLRLSCAASGF |
| | TFDDYAMHWVRQAPGKGLEWVSGISWNSGSIGYADSVKGRFTISRDNAK |
| | NSLYLQMNSLRAEDTAVYYCAKDGSSSWSWGYFDYWGQGTLVTVSSGG |
| | GGSGGGGSGGGGSSSELTQDPAVSVALGQTVRTTCQGDALRSYYASWYQ |
| | QKPGQAPMLVIYGKNNRPSGIPDRFSGSDSGDTASLTITGAQAEDEADYY |
| | CNSRDSSGYPVFGTGTKVTVLIEVMYPPPYLDNEKSNGTIIHVKGKHLCPS |
| | PLFPGPSKPFWVLVVVGGVLACYSLLVTVAFIIFWVRLKIQVRKAAITSYE |
| | KSDGVYTGLSTRNQETYETLKHEKPPQ (SEQ ID NO: 75) |

TABLE 3
Exemplary Anti-Mesothelin Antigen Binding Domain (ABD) Amino Acid
Sequences

| ABD ID | Amino Acid Sequence |
|---|---|
| M1 | QVQLQQSGAEVKKPGASVKVSCKASGYTFTGYYMHWVRQAPGQGLEWM |
| | GRINPNSGGTNYAQKFQGRVTMTRDTSISTAYMELSRLRSEDTAVYYCARG |
| | RYYGMDVWGQGTMVTVSSGGGGSGGGGSGGGGSGGGGSEIVLTQSPATL |
| | SLSPGERATISCRASQSVSSNFAWYQQRPGQAPRLLIYDASNRATGIPPRFSG |
| | SGSGTDFTLTISSLEPEDFAAYYCHQRSNWLYTFGQGTKVDIK (SEQ ID NO: |
| | 17) |
| M2 | QVQLVQSGAEVKKPGASVKVSCKASGYTFTGYYMHWVRQAPGQGLEWM |
| | GWINPNSGGTNYAQKFQGRVTMTRDTSISTAYMELSRLRSDDTAVYYCAR |
| | DLRRTVVTPRAYYGMDVWGQGTTVTVSSGGGGSGGGGSGGGGSGGGGSD |
| | IQLTQSPSTLSASVGDRVTITCQASQDISNSLNWYQQKAGKAPKLLIYDAST |
| | LETGVPSRFSGSGSGTDFSFTISSLQPEDIATYYCQQHDNLPLTFGQGTKVEI |
| | K (SEQ ID NO: 18) |
| M3 | QVQLVQSGAEVKKPGAPVKVSCKASGYTFTGYYMHWVRQAPGQGLEWM |
| | GWINPNSGGTNYAQKFQGRVTMTRDTSISTAYMELSRLRSDDTAVYYCAR |
| | GEWDGSYYYDYWGQGTLVTVSSGGGGSGGGGSGGGGSGGGGSDIVLTQT |
| | PSSLSASVGDRVTITCRASQSINTYLNWYQHKPGKAPKLLIYAASSLQSGVP |
| | SRFSGSGSGTDFTLTISSLQPEDFATYYCQQSFSPLTFGGGTKLEIK (SEQ ID |
| | NO: 19) |
| M4 | QVQLVESGGGLVQPGGSLRLSCAASGFTFSSYWMHWVRQVPGKGLVWVS |
| | RINTDGSTTTYADSVEGRFTISRDNAKNTLYLQMNSLRDDDTAVYYCVGGH |
| | WAVWGQGTTVTVSSGGGGSGGGGSGGGGSGGGGSDIQMTQSPSTLSASVG |
| | DRVTITCRASQSISDRLAWYQQKPGKAPKLLIYKASSLESGVPSRFSGSGSGT |
| | EFTLTISSLQPDDFAVYYCQQYGHLPMYTFGQGTKVEIK (SEQ ID NO: 20) |
| M5 | QVQLVQSGAEVEKPGASVKVSCKASGYTFTDYYMHWVRQAPGQGLEWM |
| | GWINPNSGGTNYAQKFQGRVTMTRDTSISTAYMELSRLRSDDTAVYYCAS |
| | GWDFDYWGQGTLVTVSSGGGGSGGGGSGGGGSGGGGSDIVMTQSPSSLSA |
| | SVGDRVTITCRASQSIRYYLSWYQQKPGKAPKLLIYTASILQNGVPSRFSGSG |
| | SGTDFTLTISSLQPEDFATYYCLQTYTTPDFGPGTKVEIK (SEQ ID NO: 21) |
| M6 | QVQLVQSGAEVKKPGASVKVSCKASGYTFTSYYMHWVRQAPGQGLEWM |
| | GIINPSGGSTSYAQKFQGRVTMTRDTSTSTVYMELSSLRSEDTAVYYCARYR |
| | LIAVAGDYYYYGMDVWGQGTMVTVSSGGGGSGGGGSGGGGSGGGGSDIQ |
| | MTQSPSSVSASVGDRVTITCRASQGVGRWLAWYQQKPGTAPKLLIYAASTL |
| | QSGVPSRFSGSGSGTDFTLTINNLQPEDFATYYCQQANSFPLTFGGGTRLEIK |
| | (SEQ ID NO: 22) |
| M7 | QVQLVQSGGGVVQPGRSLRLSCAASGFTFSSYAMHWVRQAPGKGLEWVA |
| | VISYDGSNKYYADSVKGRFTISRDNSKNTLYLQMNSLRAEDTAVYYCARW |
| | KVSSSSPAFDYWGQGTLVTVSSGGGGSGGGGSGGGGSGGGGSEIVLTQSPA |
| | TLSLSPGERAILSCRASQSVYTKYLGWYQQKPGQAPRLLIYDASTRATGIPD |
| | RFSGSGSGTDFTLTINRLEPEDFAVYYCQHYGGSPLITFGQGTRLEIK (SEQ |
| | ID NO: 23) |
| M8 | QVQLQQSGAEVKKPGASVKVSCKTSGYPFTGYSLHWVRQAPGQGLEWMG |
| | WINPNSGGTNYAQKFQGRVTMTRDTSISTAYMELSRLRSDDTAVYYCARD |
| | HYGGNSLFYWGQGTLVTVSSGGGGSGGGGSGGGGSGGGGSDIQLTQSPSSI |
| | SASVGDTVSITCRASQDSGTWLAWYQQKPGKAPNLLMYDASTLEDGVPSR |
| | FSGSASGTEFTLTVNRLQPEDSATYYCQQYNSYPLTFGGGTKVDIK (SEQ ID |
| | NO: 24) |
| M9 | QVQLVQSGAEVKKPGASVEVSCKASGYTFTSYYMHWVRQAPGQGLEWM |
| | GIINPSGGSTGYAQKFQGRVTMTRDTSTSTVHMELSSLRSEDTAVYYCARG |
| | GYSSSSDAFDIWGQGTMVTVSSGGGGSGGGGSGGGGSGGGGSDIQMTQSP |
| | PSLSASVGDRVTITCRASQDISSALAWYQQKPGTPPKLLIYDASSLESGVPSR |
| | FSGSGSGTDFTLTISSLQPEDFATYYCQQFSSYPLTFGGGTRLEIK (SEQ ID |
| | NO: 25) |
| M10 | QVQLVQSGAEVKKPGASVKVSCKASGYTFTSYGISWVRQAPGQGLEWMG |
| | WISAYNGNTNYAQKLQGRVTMTTDTSTSTAYMELRSLRSDDTAVYYCARV |
| | AGGIYYYYGMDVWGQGTTITVSSGGGGSGGGGSGGGGSGGGGSDIVMTQ |
| | TPDSLAVSLGERATISCKSSHSVLYNRNNKNYLAWYQQKPGQPPKLLFYWA |
| | STRKSGVPDRFSGSGSGTDFTLTISSLQPEDFATYFCQQTQTFPLTFGQGTRL |
| | EIN (SEQ ID NO: 26) |
| M11 | QVQLQQSGAEVKKPGASVKVSCKASGYTFTGYYMHWVRQAPGQGLEWM |
| | GWINPNSGGTNYAQNFQGRVTMTRDTSISTAYMELRRLRSDDTAVYYCAS |
| | GWDFDYWGQGTLVTVSSGGGGGGGGSGGGGSGGGGSDIRMTQSPSSLSA |
| | SVGDRVTITCRASQSIRYYLSWYQQKPGKAPKLLIYTASILQNGVPSRFSGSG |
| | SGTDFTLTISSLQPEDFATYYCLQTYTTPDFGPGTKVEIK (SEQ ID NO: 27) |
| M12 | QVQLVQSGAEVKKPGASVKVSCKASGYTFTGYYMHWVRQAPGQGLEWM |
| | GRINPNSGGTNYAQKFQGRVTMTTDTSTSTAYMELRSLRSDDTAVYYCAR |
| | TTTSYAFDIWGQGTMVTVSSGGGGSGGGGSGGGGSGGGGSDIQLTQSPSTL |
| | SASVGDRVTITCRASQSISTWLAWYQQKPGKAPNLLIYKASTLESGVPSRFS |
| | GSGSGTEFTLTISSLQPDDFATYYCQQYNTYSPYTFGQGTKLEIK (SEQ ID |
| | NO: 28) |
| M13 | QVQLVQSGGGLVKPGGSLRLSCEASGFIFSDYYMGWIRQAPGKGLEWVSYI |
| | GRSGSSMYYADSVKGRFTFSRDNAKNSLYLQMNSLRAEDTAVYYCAASPV |
| | VAATEDFQHWGQGTLVTVSSGGGGSGGGGSGGGGSGGGGSDIVMTQTPAT |
| | LSLSPGERATLSCRASQSVTSNYLAWYQQKPGQAPRLLLFGASTRATGIPDR |
| | FSGSGSGTDFTLTINRLEPEDFAMYYCQQYGSAPVTFGQGTKLEIK (SEQ ID |
| | NO: 29) |
| M14 | QVQLVQSGAEVRAPGASVKISCKASGFTFRGYYIHWVRQAPGQGLEWMGII |
| | NPSGGSRAYAQKFQGRVTMTRDTSTSTVYMELSSLRSDDTAMYYCARTAS |
| | CGGDCYYLDYWGQGTLVTVSSGGGGSGGGGSGGGGSGGGGSDIQMTQSP |
| | PTLSASVGDRVTITCRASENVNIWLAWYQQKPGKAPKLLIYKSSSLASGVPS |
| | RFSGSGSGAEFTLTISSLQPDDFATYYCQQYQSYPLTFGGGTKVDIK (SEQ |
| | ID NO: 30) |
| M15 | QVQLVQSGGGLVQPGRSLRLSCAASGFTFDDYAMHWVRQAPGKGLEWVS |
| | GISWNSGSIGYADSVKGRFTISRDNAKNSLYLQMNSLRAEDTAVYYCAKDG |
| | SSSWSWGYFDYWGQGTLVTVSSGGGGSGGGGSGGGGSSSELTQDPAVSVA |
| | LGQTVRTTCQGDALRSYYASWYQQKPGQAPMLVIYGKNNRPSGIPDRFSGS |
| | DSGDTASLTITGAQAEDEADYYCNSRDSSGYPVFGTGTKVTVL (SEQ ID |
| | NO: 31) |
| M16 | EVQLVESGGGLVQPGRSLRLSCAASGFTFDDYAMHWVRQAPGKGLEWVS |
| | GISWNSGSTGYADSVKGRFTISRDNAKNSLYLQMNSLRAEDTALYYCAKDS |
| | SSWYGGGSAFDIWGQGTMVTVSSGGGGSGGGGSGGGGSSSELTQEPAVSV |
| | ALGQTVRITCQGDSLRSYYASWYQQKPGQAPVLVIFGRSRRPSGIPDRFSGS |
| | SSGNTASLIITGAQAEDEADYYCNSRDNTANHYVFGTGTKLTVL (SEQ ID |
| | NO: 32) |
| M17 | EVQLVESGGGLVQPGRSLRLSCAASGFTFDDYAMHWVRQAPGKGLEWVS |
| | GISWNSGSTGYADSVKGRFTISRDNAKNSLYLQMNSLRAEDTALYYCAKDS |
| | SSWYGGGSAFDIWGQGTMVTVSSGGGGSGGGGSGGGGSSSELTQDPAVSV |
| | ALGQTVRITCQGDSLRSYYASWYQQKPGQAPVLVIYGKNNRPSGIPDRFSG |
| | SSSGNTASLTITGAQAEDEADYYCNSRGSSGNHYVFGTGTKVTVL (SEQ ID |
| | NO: 33) |
| M18 | QVQLVQSGGGLVQPGGSLRLSCAASGFTFSSYWMHWVRQAPGKGLVWVS |
| | RINSDGSSTSYADSVKGRFTISRDNAKNTLYLQMNSLRAEDTAVYYCVRTG |
| | WVGSYYYYMDVWGKGTTVTVSSGGGGSGGGGSGGGGSGGGGSEIVLTQS |
| | PGTLSLSPGERATLSCRASQSVSSNYLAWYQQKPGQPPRLLIYDVSTRATGIP |
| | ARFSGGGSGTDFTLTISSLEPEDFAVYYCQQRSNWPPWTFGQGTKVEIK |
| | (SEQ ID NO: 34) |
| M19 | QVQLVQSGGGVVQPGRSLRLSCAASGFTFSSYGMHWVRQAPGKGLEWVA |
| | VISYDGSNKYYADSVKGRFTISRDNSKNTLYLQMNSLRAEDTAVYYCAKG |
| | YSRYYYYGMDVWGQGTTVTVSSGGGGSGGGGSGGGGSGGGGSEIVMTQS |
| | PATLSLSPGERAILSCRASQSVYTKYLGWYQQKPGQAPRLLIYDASTRATGI |
| | PDRFSGSGSGTDFTLTINRLEPEDFAVYYCQHYGGSPLITFGQGTKVDIK |
| | (SEQ ID NO: 35) |
| M20 | QVQLVQSGGGLVQPGGSLRLSCAASGFTFSSYAMSWVRQAPGKGLEWVSA |
| | ISGSGGSTYYADSVKGRFTISRDNSKNTLYLQMNSLRAEDTAVYYCAKREA |
| | AAGHDWYFDLWGRGTLVTVSSGGGGSGGGGSGGGGSGGGGSDIRVTQSPS |
| | SLSASVGDRVTITCRASQSISSYLNWYQQKPGKAPKLLIYAASSLQSGVPSRF |
| | SGSGSGTDFTLTISSLQPEDFATYYCQQSYSIPLTFGQGTKVEIK (SEQ ID |
| | NO: 36) |

TABLE 4
Exemplary Nucleic Acid Sequences for CARs described herein

| CAR | Nucleic Acid Sequence |
|---|---|
| CTX_269 | ATGGCCTTGCCTGTCACCGCTCTGCTTCTGCCGCTGGCCCTCTTGCTGC |
| | ACGCCGCGCGTCCAGGGAGCCAGGTGCAGCTGGTCCAGTCCGGGGGTG |
| | GACTGGTGCAGCCCGGCCGTAGCCTGCGCCTGTCTTGCGCAGCGTCCG |
| | GCTTCACATTCGACGATTATGCCATGCACTGGGTCCGGCAGGCCCCAG |
| | GTAAGGGTCTTGAATGGGTGTCTGGTATTAGCTGGAACTCCGGCAGTA |
| | TCGGCTATGCCGATTCCGTAAAGGGTCGGTTTACCATTTCCCGCGACAA |
| | CGCCAAGAACAGCCTCTACCTGCAGATGAACTCTCTGCGTGCGGAAGA |
| | TACAGCAGTATACTATTGCGCCAAAGACGGCTCATCTTCCTGGTCCTGG |
| | GGCTATTTCGACTACTGGGGACAGGGGACTCTGGTGACGGTGTCTAGC |
| | GGCGGTGGAGGTAGTGGTGGCGGTGGCTCTGGGGGTGGGGGCTCCAGC |
| | TCTGAGCTGACACAGGACCCAGCTGTGTCCGTGGCGCTCGGTCAGACT |
| | GTTCGCACCACTTGTCAAGGGGACGCATTGCGGTCATACTATGCCTCTT |
| | GGTACCAACAGAAGCCAGGCCAGGCTCCCATGCTCGTTATCTATGGAA |
| | AGAACAATCGTCCATCAGGTATCCCTGATCGTTTCTCCGGCTCTGACAG |
| | TGGGGACACCGCTAGCCTGACCATCACCGGCGCGCAGGCTGAGGACGA |
| | GGCCGACTACTATTGTAACAGTCGGGACTCCTCTGGTTACCCAGTTTTC |
| | GGCACCGGCACCAAGGTGACAGTGCTGACCACGACCCCCGCCCCCAGG |
| | CCCCCTACACCCGCACCTACCATCGCTAGCCAGCCTCTCTCCCTGCGCC |
| | CGGAGGCGTGCAGGCCTGCTGCCGGAGGCGCTGTACACACCCGCGGAC |
| | TGGACTTCGCCTGTGATATCTACATTTGGGCTCCCCTGGCGGGGACATG |
| | CGGCGTTTTGCTGTTGTCCCTCGTAATTACCCTGTATTGCCGTGTTAAGT |
| | TCTCTCGGAGTGCTGATGCACCAGCGTATCAACAGGGCCAGAATCAGC |
| | TGTACAACGAGCTGAATCTCGGGAGGAGAGAAGAGTACGACGTACTG |
| | GACAAGCGTCGGGGGAGGGATCCCGAGATGGGCGGTAAGCCCCGCAG |
| | AAAGAATCCTCAAGAAGGCCTGTATAACGAGTTGCAGAAGGACAAAA |
| | TGGCAGAGGCATATAGTGAAATCGGGATGAAGGGTGAGAGGCGCCGT |
| | GGCAAAGGCCACGATGGACTCTACCAGGGCCTGTCTACTGCTACAAAG |
| | GACACGTACGACGCTCTTCATATGCAAGCCCTCCCTCCCCGCTAA (SEQ |
| | ID NO: 37) |
| CTX_293 | ATGGCGCTTCCGGTGACCGCGCTCCTGCTTCCTCTGGCGCTGTTGCTGC |
| | ATGCCGCTAGACCGGGCTCCCAAGTGCAGCTCGTCCAAAGTGGTGGCG |
| | GGCTCGTTCAACCCGGCCGCTCCCTGCGGCTGTCTTGTGCGGCATCTGG |
| | TTTCACCTTCGATGACTATGCGATGCACTGGGTCAGACAGGCTCCGGG |
| | AAAGGGGCTCGAATGGGTGTCTGGCATCTCCTGGAACTCCGGCAGCAT |
| | CGGCTACGCTGATTCTGTCAAGGGCAGATTCACCATCAGCCGCGACAA |
| | CGCTAAGAACTCCCTGTATCTCCAGATGAACAGCCTGAGGGCGGAAGA |
| | CACGGCCGTGTATTACTGCGCCAAGGACGGCAGCTCCTCATGGAGCTG |
| | GGGATATTTTGACTACTGGGGTCAAGGCACCCTGGTGACCGTAAGCAG |
| | TGGTGGCGGGGGATCCGGCGGTGGGGGCTCCGGCGGAGGGGGCTCCA |
| | GCAGTGAGCTGACTCAAGATCCTGCTGTGAGCGTGGCCTTGGGTCAGA |
| | CAGTTCGCACCACATGCCAAGGCGACGCCCTTCGCTCTTATTACGCTTC |
| | CTGGTACCAGCAAAAACCTGGGCAGGCACCTATGCTCGTCATCTATGG |
| | GAAGAATAACCGTCCATCTGGCATCCCTGACCGCTTCTCTGGCTCCGAT |
| | AGTGGCGACACCGCTTCTCTGACCATCACCGGGGCTCAGGCTGAGGAC |
| | GAGGCGGACTATTACTGTAACAGTCGCGACTCCAGCGGCTACCCTGTC |
| | TTCGGTACTGGCACAAAAGTGACCGTCCTGATTGAAGTGATGTACCCT |
| | CCGCCTTACCTGGACAACGAGAAGAGCAACGGCACGATTATCCATGTG |
| | AAAGGCAAACACCTCTGCCCTTCCCCGCTGTTCCCGGGCCCCAGTAAG |
| | CCCTTCTGGGTCCTCGTGGTCGTAGGCGGTGTACTGGCTTGTTATAGCC |
| | TGCTCGTCACCGTCGCCTTCATTATCTTCTGGGTCCGCGTAAAGTTCAG |
| | CCGCAGCGCCGATGCCCCTGCCTACCAACAGGGCCAAAATCAGCTGTA |
| | TAACGAGCTCAACCTGGGCAGAAGGGAGGAATACGACGTGCTGGACA |
| | AAAGGCGTGGACGCGATCCTGAGATGGGCGGAAAACCCAGGCGTAAG |
| | AACCCCCAGGAGGGACTGTATAACGAGCTGCAGAAGGATAAAATGGC |
| | CGAGGCTTATTCTGAGATCGGGATGAAGGGCGAAAGACGGCGTGGCA |
| | AGGGCCATGACGGTCTCTACCAAGGTCTGTCTACCGCCACCAAGGATA |
| | CCTATGACGCCCTCCATATGCAGGCCTTGCCTCCACGCTAA (SEQ ID |
| | NO: 38) |
| CTX_964 | ATGGCTCTGCCAGTCACCGCACTCCTGTTGCCCCTCGCCCTGCTCCTGC |
| | ACGCAGCCCGCCCAGGTAGTCAAGTCCAGCTGGTGCAAAGCGGCGGG |
| | GGACTGGTGCAACCTGGGAGAAGTTTGCGCCTTAGTTGTGCGGCTTCT |
| | GGATTCACATTCGACGATTATGCCATGCATTGGGTTCGCCAGGCTCCAG |
| | GTAAGGGCTTGGAATGGGTTTCTGGTATCTCTTGGAACTCAGGGTCTAT |
| | CGGGTACGCCGACAGTGTTAAGGGCCGGTTCACCATCAGCCGGGATAA |
| | CGCAAAAAACTCTCTGTATCTCCAGATGAACAGTCTGAGGGCAGAGGA |
| | TACTGCCGTTTATTACTGTGCTAAGGATGGCTCCAGCTCATGGTCCTGG |
| | GGGTATTTCGACTACTGGGGCCAGGGCACACTGGTGACCGTCAGCTCT |
| | GGAGGCGGTGGCTCCGGGGGTGGCGGATCAGGCGGAGGCGGTTCCAG |
| | CAGTGAGCTGACCCAAGACCCAGCCGTTTCTGTGGCCTTGGGGCAGAC |
| | AGTTCGGACTACCTGCCAGGGCGACGCTCTCCGCAGCTATTACGCTTCA |
| | TGGTACCAACAGAAACCCGGGCAGGCCCCCATGCTGGTCATTTACGGG |
| | AAGAACAATAGACCCAGCGGCATTCCAGATCGCTTTTCTGGCAGCGAC |
| | TCCGGCGACACAGCTTCCCTGACTATTACGGGCGCGCAAGCCGAGGAT |
| | GAAGCGGACTATTACTGCAACTCCCGCGACAGCTCCGGTTACCCGGTT |
| | TTCGGCACTGGTACCAAGGTGACCGTGCTGATCGAGGTCATGTACCCA |
| | CCGCCATATCTGGACAACGAGAAGTCCAATGGCACGATTATCCACGTC |
| | AAAGGCAAGCATCTGTGCCCCTCCCCTCTTTTCCCAGGACCTTCCAAAC |
| | CCTTCTGGGTTCTTGTCGTGGTCGGGGGCGTGCTCGCCTGCTACTCATT |
| | GCTGGTTACAGTGGCATTCATTATCTTTTGGGTTCGTCTGAAGATCCAG |
| | GTGCGCAAAGCGGCCATCACCAGTTATGAGAAGTCCGACGGAGTTTAC |
| | ACAGGCCTGTCTACCAGGAACCAGGAGACCTACGAGACCCTGAAGCAC |
| | GAGAAGCCTCCCCAGTAA (SEQ ID NO: 39) |
| CTX_760 | ATGGCACTGCCGGTGACAGCCCTGCTCCTGCCGCTGGCCCTGCTCCTTC |
| | ATGCAGCCCGTCCACAGGTTCAACTTGTCCAGAGCGGTGGCGGACTCG |
| | TCCAGCCCGGCAGGTCCCTGAGACTGTCCTGCGCTGCCTCTGGATTTAC |
| | CTTCGATGACTACGCTATGCATTGGGTCCGCCAGGCCCCAGGGAAGGG |
| | CCTCGAATGGGTCTCCGGCATCTCCTGGAACTCCGGCTCCATTGGTTAC |
| | GCCGACAGCGTGAAGGGCAGGTTTACTATTAGTAGAGATAACGCCAAG |
| | AACTCTCTCTATCTCCAGATGAACAGTCTGCGCGCAGAAGATACTGCA |
| | GTGTATTACTGCGCTAAGGACGGCAGCAGTTCCTGGAGCTGGGGCTAT |
| | TTCGATTACTGGGGACAGGGCACTCTTGTGACAGTATCCAGCGGTGGG |
| | GGCGGAAGTGGCGGAGGCGGATCCGGGGGCGGGGGTAGCTCCTCTGA |
| | GCTGACCCAGGACCCGGCCGTCTCCGTAGCTCTGGGCCAGACCGTAAG |
| | GACTACCTGTCAGGGGGACGCACTGCGCTCCTACTATGCCAGCTGGTA |
| | TCAACAGAAGCCCGGCCAAGCCCCCATGCTGGTGATCTATGGCAAAAA |
| | CAATAGGCCGAGCGGGATCCCAGATAGGTTCAGCGGTTCCGACAGCGG |
| | AGATACTGCTTCCTTGACTATCACCGGCGCGCAGGCCGAGGACGAAGC |
| | TGACTACTATTGTAATAGCCGGGACAGTAGCGGTTACCCTGTGTTCGG |
| | GACTGGCACCAAGGTGACCGTTCTGACCACTACGCCCGCGCCTAGGCC |
| | CCCAACACCAGCCCCTACTATCGCCTCTCAGCCGCTGTCTCTCAGGCCT |
| | GAAGCGTGTAGGCCGGCTGCCGGCGGGGCTGTCCACACTCGCGGCCTT |
| | GATTTCGCTTGCGATATCTATATCTGGGCCCCGCTGGCGGGGACATGCG |
| | GGGTCTTGCTTCTGAGCCTCGTCATCACCTTGTACTGCCGTGTGAAATT |
| | TTCCCGCTCTGCTGACGCCCCCGCCTACCAACAGGGCCAGAACCAACT |
| | GTATAATGAGCTGAACCTGGGCCGCAGAGAAGAGTATGATGTCCTGGA |
| | CAAGCGTCGCGGCCGGGATCCGGAGATGGGCGGTAAGCCCCGTAGAA |
| | AGAACCCCCAGGAGGGTCTGTATAACGAGCTGCAGAAGGATAAGATG |
| | GCTGAGGCGTACAGCGAAATTGGCATGAAGGGAGAGCGGCGCAGAGG |
| | GAAAGGTCATGACGGCCTGTATCAAGGCCTCTCCACCGCTACTAAAGA |
| | TACCTATGATGCTTTGCATATGCAGGCGCTGCCCCCACGTCGCGCCAAG |
| | AGATCCGGCTCCGGCGCCACCAATTTCTCTCTTCTCAAACAGGCCGGA |
| | GACGTGGAAGAGAACCCCGGCCCTATGATTGAAACGTATAATCAGACG |
| | TCTCCCCGGAGCGCTGCCACCGGTTTGCCCATTTCTATGAAGATTTTCA |
| | TGTACTTGCTGACGGTCTTTTTGATCACTCAAATGATCGGTTCTGCTCT |
| | GTTCGCAGTATACCTGCATCGGCGCCTGGATAAAATTGAGGACGAGCG |
| | GAACCTGCACGAAGATTTCGTCTTCATGAAAACCATTCAGCGCTGTAA |
| | CACTGGTGAGAGGTCTTTGTCCCTCCTGAATTGCGAGGAAATCAAATC |
| | CCAGTTTGAGGGCTTCGTGAAAGACATCATGCTGAACAAAGAGGAAAC |
| | CAAAAAGGAGAATAGTTTTGAAATGCAGAAGGGAGATCAAAATCCCC |
| | AGATTGCTGCCCATGTCATCAGTGAGGCCAGCTCTAAAACCACATCCG |
| | TCCTGCAGTGGGCCGAGAAGGGTTATTACACTATGAGTAACAATTTGG |
| | TGACCCTGGAGAACGGCAAGCAGCTGACTGTCAAGCGCCAGGGCCTGT |
| | ACTATATCTACGCTCAGGTTACGTTCTGCTCCAATCGTGAGGCCTCTAG |
| | CCAAGCACCGTTTATTGCATCCCTGTGTCTGAAGTCCCCCGGTCGTTTC |
| | GAGCGCATCCTCCTGCGCGCTGCCAACACCCACTCATCCGCCAAGCCC |
| | TGTGGACAACAGTCTATCCATCTTGGGGGCGTGTTTGAGCTCCAGCCCG |
| | GCGCCTCTGTGTTCGTCAATGTTACTGACCCCTCTCAGGTGAGCCATGG |
| | CACCGGTTTTACCAGTTTTGGTCTTCTGAAGCTGTAA (SEQ ID NO: 40) |
| CTX_761 | ATGGCCCTGCCTGTGACCGCGTTGCTCCTGCCCCTCGCCCTGTTGCTTC |
| | ATGCCGCTCGTCCCCAGGTGCAGCTCGTCCAGAGCGGCGGAGGTCTGG |
| | TGCAACCCGGAAGGAGCCTGCGCCTGAGCTGTGCTGCCTCCGGTTTCA |
| | CTTTCGATGACTATGCCATGCACTGGGTCAGGCAGGCTCCGGGCAAGG |
| | GTCTGGAATGGGTCTCTGGGATCAGCTGGAACTCAGGATCCATCGGGT |
| | ACGCTGATTCTGTCAAGGGGAGATTCACTATTTCACGCGATAACGCTA |
| | AGAACAGTCTCTACCTGCAGATGAACTCTCTCCGCGCAGAGGATACTG |
| | CTGTCTATTACTGTGCGAAGGACGGAAGTTCCAGCTGGAGCTGGGGTT |
| | ATTTTGATTATTGGGGCCAGGGCACCCTGGTGACCGTCAGTTCCGGAG |
| | GCGGAGGCTCCGGCGGTGGAGGCTCCGGGGGCGGAGGCAGCTCCTCA |
| | GAACTCACCCAAGACCCTGCGGTAAGTGTCGCATTGGGCCAGACCGTT |
| | CGTACCACTTGCCAGGGCGACGCCCTGCGTAGCTACTATGCGAGTTGG |
| | TACCAACAGAAGCCAGGCCAGGCCCCTATGCTCGTGATCTATGGCAAA |
| | AATAACCGTCCTAGCGGTATTCCAGACCGGTTTTCTGGTAGCGATAGC |
| | GGCGATACCGCAAGCTTGACTATCACAGGCGCCCAGGCCGAAGACGA |
| | AGCCGATTATTACTGTAACAGTCGCGACTCATCCGGCTATCCTGTCTTT |
| | GGCACGGGTACTAAAGTCACTGTGCTTATCGAGGTTATGTACCCGCCA |
| | CCGTATCTGGACAACGAGAAGAGCAACGGTACGATTATCCACGTTAAG |
| | GGCAAGCACCTCTGCCCCAGTCCTCTGTTCCCAGGACCATCCAAGCCCT |
| | TCTGGGTCCTGGTCGTTGTGGGAGGGGTCCTTGCATGCTACAGTCTTCT |
| | GGTGACAGTGGCCTTCATTATCTTTTGGGTGCGTGTGAAGTTCAGCCGC |
| | AGTGCTGACGCCCCCGCATATCAACAGGGCCAAAATCAGCTGTACAAT |
| | GAGCTGAACCTCGGAAGGCGCGAAGAGTACGACGTGCTGGACAAGAG |
| | ACGCGGGCGCGACCCTGAGATGGGCGGTAAGCCTAGACGCAAGAACC |
| | CCCAGGAAGGACTCTACAACGAACTGCAAAAGGATAAGATGGCTGAG |
| | GCCTACAGCGAGATCGGCATGAAGGGCGAACGGAGACGCGGCAAGGG |
| | TCATGACGGCCTTTACCAAGGGCTGTCAACAGCGACAAAAGATACGTA |
| | CGACGCATTGCACATGCAGGCCCTGCCACCTCGCAGGGCTAAACGCAG |
| | TGGCAGCGGTGCCACCAATTTTAGCCTTCTGAAGCAGGCCGGCGATGT |
| | GGAGGAAAACCCTGGACCCATGATCGAAACATATAACCAGACATCCCC |
| | CAGGTCTGCCGCAACCGGTCTGCCCATCAGCATGAAGATTTTTATGTAC |
| | CTGCTTACCGTGTTTTTGATCACACAGATGATCGGCAGCGCTCTCTTCG |
| | CCGTTTACCTCCACCGGCGTCTGGACAAGATTGAGGACGAGCGCAACC |
| | TTCACGAGGATTTCGTCTTTATGAAGACTATTCAGAGGTGCAATACTGG |
| | CGAGCGTTCCCTCTCCCTGCTCAACTGCGAAGAGATCAAGTCCCAATTC |
| | GAAGGCTTTGTAAAGGATATCATGCTGAACAAGGAAGAGACGAAGAA |
| | AGAGAACAGCTTTGAGATGCAGAAAGGTGATCAGAACCCTCAGATCGC |
| | TGCCCACGTCATTAGCGAGGCCAGTTCCAAGACAACCTCCGTCTTGCA |
| | GTGGGCCGAGAAGGGTTATTACACCATGTCCAATAACCTGGTGACGCT |
| | GGAAAACGGCAAGCAGCTGACAGTGAAGCGCCAGGGGCTGTACTATA |
| | TCTATGCTCAGGTGACTTTCTGCTCTAACAGAGAGGCATCCTCTCAGGC |
| | CCCATTTATCGCAAGCCTGTGCCTGAAGTCCCCCGGCCGGTTTGAAAG |
| | GATCCTTCTCCGCGCCGCAAACACCCATTCCAGCGCTAAACCATGTGG |
| | CCAACAGTCTATCCACCTGGGAGGCGTCTTCGAGCTGCAGCCTGGCGC |
| | ATCTGTGTTTGTCAACGTGACCGATCCCTCTCAGGTTTCCCATGGCACC |
| | GGCTTCACCAGTTTTGGGCTCTTGAAACTTTAA (SEQ ID NO: 41) |
| CTX_762 | ATGGCCCTGCCGGTCACTGCCCTGCTCCTGCCCCTGGCCTTGCTCCTGC |
| | ACGCCGCTCGCCCACAGGTACAGCTTGTGCAGAGCGGCGGTGGCCTCG |
| | TCCAGCCGGGCCGTTCTCTCCGCCTGTCATGCGCAGCTTCCGGCTTTAC |
| | GTTCGACGATTATGCTATGCATTGGGTGAGGCAGGCCCCGGGCAAGGG |
| | TCTGGAGTGGGTGAGCGGCATCTCCTGGAATAGCGGTTCCATCGGCTA |
| | TGCGGATTCCGTCAAAGGCCGCTTTACAATCTCTAGGGATAACGCAAA |
| | AAATTCCCTGTACCTCCAGATGAACAGCCTCCGTGCTGAGGATACCGC |
| | TGTGTACTATTGCGCTAAGGACGGTTCTAGCTCCTGGTCCTGGGGCTAC |
| | TTCGACTACTGGGGCCAGGGCACTCTGGTCACCGTCTCTTCCGGAGGG |
| | GGTGGCAGCGGTGGAGGCGGAAGCGGAGGCGGAGGCTCTAGCAGTGA |
| | GCTGACCCAGGACCCTGCTGTGTCTGTCGCCCTGGGCCAGACAGTCAG |
| | AACCACTTGCCAGGGTGATGCTCTCCGCAGCTATTACGCGTCCTGGTAC |
| | CAACAGAAGCCCGGACAGGCCCCTATGCTTGTGATTTATGGAAAGAAT |
| | AACCGGCCTAGTGGTATCCCCGATAGGTTCTCAGGATCCGACTCCGGT |
| | GACACCGCCAGCCTGACCATCACTGGAGCACAGGCCGAGGATGAAGC |
| | CGATTATTACTGCAACTCCCGCGACAGCTCCGGTTACCCAGTGTTTGGA |
| | ACCGGAACCAAAGTGACCGTGCTCATCGAGGTGATGTACCCGCCCCCG |
| | TACCTGGATAACGAAAAGAGCAACGGAACCATTATCCACGTGAAGGG |
| | AAAACACCTGTGCCCCAGCCCTCTCTTCCCAGGGCCAAGTAAGCCTTTC |
| | TGGGTGCTGGTAGTGGTTGGCGGTGTACTGGCCTGCTACTCCCTCCTGG |
| | TGACCGTTGCCTTCATTATCTTCTGGGTCATGGCTGCAGGAGGCCCGGG |
| | AGCTGGCTCCGCAGCCCCGGTATCTAGCACTTCCAGCCTCCCCCTTGCC |
| | GCGCTCAACATGCGTGTGCGGAGGCGCCTCTCCCTGTTCTTGAACGTGC |
| | GTACGCAAGTGGCTGCCGATTGGACTGCGCTGGCCGAGGAAATGGATT |
| | TCGAGTATCTGGAGATTAGGCAGCTGGAAACCCAGGCTGATCCCACAG |
| | GGAGGCTCCTGGACGCCTGGCAGGGTCGCCCCGGTGCAAGTGTGGGCC |
| | GTCTTCTGGAGTTGCTGACAAAGCTGGGACGCGATGACGTGCTTTTGG |
| | AGTTGGGCCCGTCCATTGAAGAGGACTGCCAGAAGTACATCTTGAAAC |
| | AGCAACAGGAAGAGGCAGAGAAACCTTTGCAGGTCGCCGCGGTTGAC |
| | AGTAGCGTGCCTCGCACCGCCGAGCTTGCTGGCATCACGACCCTGGAT |
| | GACCCGCTGGGCCATATGCCCGAGCGTTTTGACGCGTTCATCTGCTACT |
| | GCCCCAGCGACATTCGTGTGAAATTCTCACGCTCTGCAGATGCGCCAG |
| | CTTACCAACAGGGCCAGAACCAGCTGTACAACGAGCTTAACCTGGGAC |
| | GTAGAGAGGAATACGACGTCCTGGACAAAAGGAGAGGGCGCGATCCC |
| | GAGATGGGTGGCAAGCCCCGTCGCAAGAACCCTCAAGAGGGTCTCTAC |
| | AATGAGTTGCAAAAGGACAAGATGGCAGAGGCATACTCTGAAATTGG |
| | CATGAAGGGCGAGAGGCGTAGAGGAAAGGGTCACGACGGCCTCTACC |
| | AGGGCCTGTCCACCGCTACCAAGGATACCTACGATGCCCTGCACATGC |
| | AGGCTCTCCCTCCAAGGTAA (SEQ ID NO: 42) |
| CTX_763 | ATGGCCTTGCCTGTGACCGCATTGCTGCTTCCACTGGCTCTCTTGCTCC |
| | ACGCCGCGAGACCTCAGGTGCAGCTGGTTCAGAGCGGAGGTGGGCTGG |
| | TCCAACCCGGTCGCTCTTTGAGACTGTCCTGTGCTGCGTCTGGATTTAC |
| | ATTCGATGACTATGCAATGCACTGGGTTCGTCAGGCCCCTGGCAAGGG |
| | CCTGGAGTGGGTGTCCGGCATCAGCTGGAACTCCGGATCCATCGGATA |
| | CGCTGATAGTGTAAAAGGCCGTTTCACCATCTCCCGCGACAACGCAAA |
| | GAATAGTCTGTATCTCCAGATGAATAGCCTCCGCGCTGAGGATACCGC |
| | TGTGTACTATTGCGCCAAAGACGGTTCCTCTAGCTGGTCTTGGGGCTAC |
| | TTTGATTACTGGGGCCAGGGGACACTGGTGACCGTCAGCTCCGGCGGT |
| | GGCGGTAGCGGGGGTGGCGGATCCGGTGGCGGTGGCTCCAGTTCCGAG |
| | CTGACCCAGGATCCGGCCGTTTCTGTTGCCCTGGGGCAGACTGTGAGG |
| | ACAACCTGTCAGGGGGACGCTCTGAGATCTTATTACGCCTCTTGGTATC |
| | AACAGAAACCCGGCCAGGCCCCTATGCTGGTGATCTACGGAAAGAATA |
| | ACCGCCCATCAGGCATCCCTGACCGTTTCTCCGGCAGCGACTCAGGCG |
| | ACACTGCTTCCTTGACCATCACCGGAGCGCAAGCTGAGGATGAAGCCG |
| | ACTACTATTGCAACTCCCGCGACAGCTCCGGTTATCCTGTCTTCGGAAC |
| | CGGCACTAAGGTGACCGTGTTGATTGAGGTGATGTACCCGCCCCCATA |
| | TCTCGACAACGAGAAATCTAATGGAACCATTATCCACGTGAAGGGCAA |
| | GCACCTCTGCCCGAGCCCTCTTTTCCCGGGTCCATCAAAGCCATTCTGG |
| | GTGCTGGTGGTAGTCGGCGGTGTGCTGGCTTGCTACTCCCTCCTGGTCA |
| | CTGTCGCGTTTATTATCTTTTGGGTGATGGCCGCTGGAGGCCCAGGCGC |
| | CGGTAGCGCTGCGCCGGTTTCCTCAACATCAAGCCTTCCTCTGGCCGCA |
| | CTGAACATGCGTGTGAGGCGCAGACTCTCTCTCTTTTTGAACGTGAGGA |
| | CTCAGGTCGCCGCTGACTGGACCGCCCTGGCCGAAGAGATGGACTTCG |
| | AATACCTTGAAATCCGCCAGCTCGAGACCCAAGCCGACCCCACTGGTA |
| | GGTTGCTTGACGCCTGGCAAGGGCGCCCCGGCGCTAGCGTGGGCAGGC |
| | TTCTGGAGTTGCTGACAAAGCTGGGCCGCGACGATGTGCTCCTGGAGC |
| | TGGGCCCCTCCATCGAGGAAGACTGCCAGAAATACATCCTCAAGCAAC |
| | AGCAAGAAGAGGCTGAGAAGCCTCTGCAAGTGGCCGCTGTCGACAGCT |
| | CCGTGCCACGCACGGCTGAGCTTGCTGGCATCACAACCCTTGACGATC |
| | CTCTGGGCCATATGCCAGAGAGGTTCGACGCGTTCATTTGTTACTGCCC |
| | ATCCGACATTAAAAAGGTCGCCAAGAAACCCACCAACAAGGCACCTCA |
| | TCCGAAGCAGGAGCCACAGGAGATTAATTTCCCTGATGACCTGCCCGG |
| | GTCCAATACTGCCGCACCGGTGCAGGAAACCTTGCATGGTTGTCAGCC |
| | CGTCACCCAGGAGGATGGCAAGGAGTCCCGGATTTCTGTGCAGGAGCG |
| | CCAGCGCGTTAAGTTCTCCCGCAGCGCCGACGCCCCTGCCTACCAACA |
| | GGGCCAAAACCAGCTCTACAATGAGCTTAACCTGGGACGCCGTGAAGA |
| | GTACGACGTGTTGGACAAACGCCGGGGTCGCGACCCTGAAATGGGCGG |
| | AAAGCCTAGGCGTAAGAACCCTCAGGAAGGTCTGTACAATGAACTGCA |
| | GAAGGATAAGATGGCCGAAGCCTATTCTGAGATCGGAATGAAAGGAG |
| | AACGCCGGCGCGGCAAGGGCCATGACGGCCTGTATCAGGGCTTGTCCA |
| | CGGCTACTAAAGACACCTACGACGCATTGCACATGCAGGCGCTGCCCC |
| | CACGCTAA (SEQ ID NO: 43) |
| CTX_764 | ATGGCGCTGCCCGTCACAGCCCTCCTGCTTCCCCTGGCTCTCTTGCTGC |
| | ACGCCGCTAGACCCCAGGTTCAGTTGGTGCAGTCTGGAGGCGGTCTCG |
| | TTCAACCTGGGCGCTCCCTGAGGCTCAGTTGTGCGGCCAGCGGCTTCAC |
| | TTTCGATGACTACGCTATGCACTGGGTGCGCCAGGCCCCAGGCAAGGG |
| | CCTGGAGTGGGTCTCTGGTATCAGCTGGAACTCCGGCAGCATTGGCTA |
| | CGCCGACTCTGTGAAAGGGCGCTTCACAATCAGCCGCGACAACGCAAA |
| | AAACTCCCTGTACCTGCAGATGAATAGCCTTAGAGCTGAGGACACAGC |
| | CGTGTATTACTGCGCCAAAGACGGAAGCTCCTCTTGGAGCTGGGGTTA |
| | CTTTGATTATTGGGGCCAGGGTACCCTGGTGACCGTGTCTTCAGGCGGT |
| | GGGGGCAGCGGTGGAGGCGGTTCTGGGGGCGGAGGCTCCTCTAGCGA |
| | GCTGACCCAAGACCCCGCCGTGTCCGTGGCCCTTGGACAGACCGTGCG |
| | TACGACCTGCCAAGGCGATGCGCTGCGCAGCTATTACGCGTCTTGGTA |
| | TCAACAGAAGCCTGGCCAGGCCCCCATGCTGGTCATCTACGGCAAGAA |
| | CAATCGCCCAAGCGGGATCCCCGATCGCTTCTCCGGCTCAGACAGTGG |
| | AGACACTGCATCCCTGACTATCACGGGCGCCCAGGCAGAAGACGAGGC |
| | CGACTATTACTGCAACAGCAGGGACAGCTCCGGCTATCCCGTTTTTGG |
| | GACTGGTACAAAAGTTACAGTGCTTACTACCACTCCCGCACCTAGGCC |
| | CCCGACCCCAGCACCTACCATCGCGTCTCAACCTCTGTCCCTGCGGCCA |
| | GAGGCATGTCGTCCCGCTGCGGGTGGCGCAGTGCATACCCGCGGACTG |
| | GATTTCGCTTGCGACATCTATATTTGGGCGCCCCTTGCCGGCACCTGTG |
| | GCGTTCTGCTCCTGTCACTGGTCATCACTCTGTACTGTATGGCAGCCGG |
| | AGGCCCGGGCGCTGGCTCTGCTGCCCCAGTGAGCTCCACTAGCTCATT |
| | GCCTCTGGCTGCACTGAACATGCGTGTGCGCCGGCGTCTGAGTCTCTTC |
| | CTTAACGTGCGCACTCAGGTTGCTGCCGACTGGACCGCCCTGGCGGAA |
| | GAGATGGACTTCGAATATCTGGAGATCCGGCAGCTCGAGACCCAGGCC |
| | GACCCAACAGGACGTCTCCTGGACGCCTGGCAGGGACGCCCTGGCGCC |
| | AGCGTGGGTCGTCTGCTCGAGCTTCTGACTAAGCTGGGTCGCGACGAT |
| | GTCCTGCTCGAGCTCGGCCCGTCTATCGAAGAGGACTGTCAGAAGTAC |
| | ATCCTCAAGCAGCAACAGGAAGAGGCAGAAAAGCCCCTCCAGGTGGC |
| | TGCCGTGGATTCAAGCGTGCCGCGTACCGCGGAGCTGGCCGGCATCAC |
| | TACCCTGGATGACCCTCTGGGTCACATGCCCGAACGTTTCGACGCTTTC |
| | ATCTGCTATTGTCCGAGTGACATCAAAAAGGTTGCCAAAAAGCCAACC |
| | AACAAGGCGCCGCACCCCAAGCAAGAGCCTCAAGAGATCAACTTCCC |
| | GGACGATCTGCCTGGCAGTAACACGGCCGCACCGGTCCAGGAAACCCT |
| | GCACGGTTGTCAGCCTGTCACCCAGGAGGACGGAAAGGAGTCCCGCAT |
| | CAGTGTGCAGGAGCGGCAGCGCGTGAAGTTCAGCCGCTCCGCCGACGC |
| | GCCAGCCTACCAACAGGGTCAGAACCAGCTGTATAACGAGTTGAACCT |
| | GGGCAGGAGAGAAGAGTATGATGTGCTGGACAAGAGAAGGGGCCGCG |
| | ACCCTGAGATGGGCGGTAAGCCTCGCCGTAAGAACCCACAAGAGGGC |
| | CTGTACAACGAGCTGCAGAAGGATAAGATGGCCGAGGCTTACTCTGAG |
| | ATTGGTATGAAGGGCGAGCGCAGGCGCGGCAAGGGCCATGATGGCTT |
| | GTACCAGGGCTTGTCAACCGCTACCAAGGATACCTACGACGCTTTGCA |
| | TATGCAGGCTCTGCCTCCCCGCTGA (SEQ ID NO: 44) |
| CTX_271 | ATGGCCCTGCCCGTGACCGCGCTGCTCCTTCCCCTCGCCCTTCTCCTGC |
| | ACGCGGCCAGACCCGGCAGCGAAGTCCAGCTGGTGGAGAGCGGCGGA |
| | GGCCTGGTGCAGCCGGGAAGGTCCCTGCGCTTGTCATGTGCAGCCTCC |
| | GGCTTCACATTCGATGACTACGCCATGCACTGGGTGCGCCAGGCCCCG |
| | GGAAAGGGACTGGAGTGGGTCAGCGGCATCAGCTGGAATTCCGGCTCT |
| | ACCGGCTACGCGGACTCTGTTAAGGGGCGCTTCACGATCAGTCGCGAC |
| | AATGCGAAGAATTCCCTGTACCTGCAGATGAACAGTCTGCGCGCCGAA |
| | GACACCGCCCTGTACTATTGTGCCAAGGATTCATCCTCATGGTATGGCG |
| | GGGGATCCGCCTTTGATATTTGGGGGCAGGGTACTATGGTTACCGTCTC |
| | CTCTGGGGGCGGAGGGTCTGGAGGGGGCGGATCTGGCGGTGGCGGGA |
| | GCTCCAGCGAGCTGACTCAGGACCCCGCCGTTAGCGTCGCTTTGGGAC |
| | AGACCGTCCGGATCACCTGCCAGGGTGATTCCCTGCGCTCTTATTACGC |
| | CTCCTGGTATCAACAGAAGCCCGGCCAGGCCCCTGTCCTGGTCATTTAC |
| | GGCAAGAATAACCGTCCAAGTGGCATCCCCGATCGCTTTTCCGGCAGT |
| | TCCTCTGGCAACACTGCAAGTCTCACCATCACTGGCGCCCAGGCAGAG |
| | GATGAAGCAGATTATTACTGTAATTCTAGGGGCTCCTCTGGTAACCATT |
| | ACGTGTTCGGCACCGGGACCAAGGTAACTGTGCTGACTACAACCCCCG |
| | CGCCCAGGCCCCCTACTCCTGCGCCCACAATCGCTTCACAGCCTCTCAG |
| | CCTGCGCCCCGAGGCTTGTAGGCCTGCCGCGGGCGGTGCGGTGCACAC |
| | GCGCGGCCTGGACTTCGCTTGCGATATCTATATCTGGGCTCCACTGGCT |
| | GGCACCTGTGGGGTCCTTTTGCTGAGCCTGGTAATTACTCTGTATTGTC |
| | GCGTGAAGTTCTCCCGCAGTGCTGACGCCCCTGCCTACCAACAGGGAC |
| | AGAACCAGCTGTACAATGAGTTGAACCTCGGACGTCGCGAAGAGTATG |
| | ACGTGCTCGACAAGCGTCGCGGCCGCGACCCTGAGATGGGAGGTAAAC |
| | CTAGGCGCAAGAATCCGCAGGAAGGCCTGTACAACGAACTTCAGAAG |
| | GATAAAATGGCTGAGGCCTATTCCGAAATTGGCATGAAGGGAGAGCGC |
| | CGTCGGGGGAAAGGACACGATGGACTGTACCAGGGTCTCAGCACCGCC |
| | ACCAAGGACACGTACGACGCCCTGCATATGCAGGCTCTGCCTCCCCGC |
| | TAA (SEQ ID NO: 45) |
| CTX_1120 | ATGGCGCTGCCCGTGACCGCCCTTCTGCTCCCTCTGGCCCTGCTTCTGC |
| | ACGCCGCTCGCCCAGGTTCTCAGGTTCAACTGGTCCAAAGCGGAGGGG |
| | GCCTCGTGCAGCCAGGCCGTTCCTTGCGTCTGAGCTGCGCCGCTTCCGG |
| | GTTCACTTTCGATGACTACGCCATGCACTGGGTGCGCCAGGCCCCCGG |
| | GAAGGGACTGGAGTGGGTCTCCGGCATCTCATGGAACAGCGGCTCAAT |
| | CGGCTACGCTGACAGCGTCAAGGGCCGTTTTACCATCTCCAGAGACAA |
| | CGCCAAGAATTCTCTGTACCTTCAGATGAACAGCCTCCGTGCTGAGGA |
| | CACGGCAGTTTATTACTGCGCCAAGGACGGTAGCTCTAGCTGGTCATG |
| | GGGGTACTTTGACTACTGGGGCCAGGGCACCCTGGTGACTGTAAGTAG |
| | CGGCGGTGGCGGGTCAGGTGGCGGTGGGTCTGGAGGGGGTGGGAGCT |
| | CCAGCGAGCTCACTCAGGATCCCGCAGTGTCCGTGGCCCTGGGGCAAA |
| | CTGTGAGGACTACATGTCAGGGAGACGCCCTCCGGAGCTATTACGCCT |
| | CCTGGTACCAACAGAAGCCCGGACAGGCTCCCATGCTGGTGATTTATG |
| | GGAAGAATAACAGACCCTCCGGGATTCCCGACAGGTTCTCCGGGTCTG |
| | ACTCCGGCGATACGGCTTCTCTGACAATCACCGGTGCCCAGGCCGAGG |
| | ACGAGGCCGACTACTATTGTAACTCACGCGATTCTAGCGGATATCCAG |
| | TGTTCGGCACCGGCACTAAGGTGACAGTGCTGATTGAGGTGATGTACC |
| | CTCCCCCATACCTGGACAACGAAAAGAGCAACGGCACCATCATTCATG |
| | TCAAGGGCAAGCATCTGTGTCCTAGCCCCCTGTTCCCTGGCCCTAGCAA |
| | GCCTTTCTGGGTGCTTGTGGTCGTGGGCGGAGTGCTGGCTTGTTATTCC |
| | CTGCTTGTTACAGTTGCATTCATTATCTTTTGGGTGATGGCGGCTGGTG |
| | GCCCAGGTGCTGGCTCCGCTGCCCCAGTGTCATCCACCTCTTCCCTGCC |
| | GCTGGCGGCCCTGAACATGCGGGTGCGCCGTCGGCTGTCTCTGTTCCTG |
| | AACGTTCGCACGCAGGTCGCCGCTGACTGGACTGCCCTTGCGGAGGAA |
| | ATGGACTTTGAGTATCTGGAAATCCGCCAGCTGGAAACTCAGGCCGAT |
| | CCAACTGGCCGTCTGCTTGACGCTTGGCAGGGTCGCCCTGGCGCTAGC |
| | GTGGGCAGACTCTTGGAGTTGCTCACAAAACTGGGTCGTGATGACGTG |
| | TTGCTCGAGCTGGGCCCTTCCATCGAGGAAGACTGTCAGAAGTATATC |
| | CTTAAGCAGCAACAGGAAGAGGCTGAGAAACCGTTGCAAGTGGCTGC |
| | AGTTGACTCCAGCGTACCGCGTACCGCTGAATTGGCCGGCATCACTAC |
| | GCTGGATGACCCACTGGGCCACATGCCTGAGCGCTTTGACGCTTTCATC |
| | TGTTACTGCCCTAGCGACATCAAAAAGGTGGCCAAAAAGCCTACCAAC |
| | AAGGCCCCTCATCCCAAGCAGGAACCGCAAGAGATCAACTTCCCGGAC |
| | GATCTGCCCGGGTCTAACACCGCCGCGCCTGTGCAGGAAACCCTGCAC |
| | GGCTGCCAGCCAGTGACGCAGGAGGACGGCAAGGAGTCCAGGATCTC |
| | TGTCCAGGAGAGGCAGAGGCTGAAGATCCAAGTCCGTAAAGCGGCCA |
| | TCACAAGTTATGAGAAATCCGACGGTGTGTACACCGGGCTGAGCACAA |
| | GAAACCAGGAGACTTATGAAACCCTCAAGCACGAGAAACCCCCTCAGT |
| | AA (SEQ ID NO: 46) |
| CTX_1114 | ATGGCTCTCCCTGTGACCGCTCTGCTTCTGCCTCTTGCCCTCCTGTTGCA |
| | CGCTGCCCGGCCTGAAGTCCAGCTGGTTGAGAGCGGTGGAGGCCTGGT |
| | GCAGCCCGGACGGTCCCTGCGCTTGAGCTGTGCTGCCTCCGGCTTCACC |
| | TTTGATGACTACGCTATGCACTGGGTTCGCCAGGCCCCCGGCAAGGGA |
| | CTGGAGTGGGTGAGCGGCATTTCCTGGAACAGCGGATCCACCGGCTAT |
| | GCCGACTCAGTGAAAGGTCGCTTCACAATTTCCAGAGACAACGCTAAG |
| | AACTCCCTTTACCTGCAGATGAACAGCTTGCGGGCGGAGGACACTGCC |
| | CTCTACTATTGTGCTAAGGATAGCTCATCCTGGTACGGCGGAGGTAGT |
| | GCCTTCGACATCTGGGGACAAGGTACGATGGTAACTGTGAGTTCCGGC |
| | GGAGGCGGATCAGGCGGAGGTGGCTCTGGCGGTGGCGGATCCAGCTCC |
| | GAGCTTACCCAGGACCCAGCCGTGAGCGTGGCCCTGGGCCAGACGGTC |
| | CGCATCACGTGCCAAGGTGATTCACTTAGAAGCTATTACGCTTCCTGGT |
| | ACCAACAGAAGCCCGGGCAGGCCCCCGTCTTGGTAATCTACGGCAAGA |
| | ATAACAGACCCTCTGGTATCCCCGACCGCTTTTCTGGTTCCTCTAGCGG |
| | TAACACCGCTTCCCTGACAATCACAGGCGCTCAGGCCGAAGACGAAGC |
| | AGATTACTATTGCAACAGCCGCGGCTCAAGTGGCAACCACTACGTCTT |
| | TGGAACCGGCACTAAGGTGACCGTACTGATTGAGGTGATGTACCCTCC |
| | CCCTTACCTGGATAACGAAAAGAGCAACGGCACTATTATCCACGTGAA |
| | AGGGAAACACCTGTGCCCTTCACCCCTGTTCCCGGGCCCTAGCAAACC |
| | TTTCTGGGTGCTGGTGGTCGTGGGCGGTGTGCTGGCCTGTTACTCCCTG |
| | CTTGTCACCGTAGCTTTTATTATCTTTTGGGTCCGCGTCAAGTTCAGCC |
| | GCAGCGCCGATGCCCCGGCATATCAACAGGGCCAGAACCAGTTGTATA |
| | ACGAACTTAACCTGGGACGTCGGGAGGAATACGACGTTCTGGACAAGC |
| | GCCGGGGCCGGGATCCCGAGATGGGTGGAAAGCCTCGTCGCAAGAAC |
| | CCGCAGGAAGGCCTGTACAATGAGTTGCAGAAAGATAAGATGGCCGA |
| | GGCTTATTCTGAAATCGGCATGAAAGGTGAACGTAGACGTGGTAAAGG |
| | CCACGACGGTCTGTACCAGGGTCTGAGTACCGCGACCAAGGACACATA |
| | CGATGCCCTGCACATGCAGGCCCTGCCGCCCCGTTAG SEQ ID NO: 47) |
| CTX_1115 | ATGGCGCTGCCAGTTACGGCACTGTTGCTCCCTCTCGCGCTGTTGCTCC |
| | ATGCCGCTAGGCCCGAGGTGCAGCTGGTCGAGTCTGGGGGCGGGCTTG |
| | TGCAGCCCGGGAGGTCCCTCCGCCTGAGCTGTGCTGCCTCTGGTTTTAC |
| | CTTTGACGATTACGCCATGCACTGGGTCAGACAGGCTCCTGGCAAAGG |
| | CCTCGAGTGGGTTAGCGGGATCTCTTGGAATTCTGGGTCCACTGGTTAC |
| | GCCGACTCCGTCAAGGGCCGTTTCACGATTTCTCGCGATAACGCGAAG |
| | AATTCCTTGTATCTCCAGATGAATTCCCTTCGCGCTGAGGACACCGCTC |
| | TGTACTATTGCGCCAAGGACTCTAGTTCCTGGTACGGGGGTGGCAGCG |
| | CGTTCGACATCTGGGGGCAAGGCACAATGGTCACAGTCAGCTCTGGCG |
| | GAGGCGGTAGCGGTGGCGGTGGCAGTGGCGGAGGCGGATCTTCCTCTG |
| | AGCTGACGCAGGACCCGGCTGTTTCTGTCGCTCTCGGGCAGACCGTCC |
| | GCATCACCTGCCAGGGCGACAGTCTGCGCTCCTATTACGCGTCCTGGTA |
| | CCAACAGAAGCCTGGTCAAGCCCCGGTCCTGGTGATCTATGGCAAAAA |
| | TAACAGACCCTCCGGGATTCCGGATCGGTTCAGTGGTTCCAGCTCTGGC |
| | AATACTGCTTCACTCACTATTACCGGCGCTCAGGCCGAAGATGAGGCA |
| | GACTATTACTGTAACTCTCGGGGAAGCTCCGGCAATCACTACGTGTTTG |
| | GCACAGGCACCAAGGTTACCGTGCTGATTGAGGTGATGTATCCTCCCC |
| | CATACCTGGACAATGAGAAGTCCAACGGAACCATTATCCACGTCAAGG |
| | GTAAGCATCTTTGCCCTAGTCCCCTCTTCCCAGGCCCTTCCAAGCCCTT |
| | CTGGGTCCTGGTGGTTGTCGGCGGTGTGCTGGCCTGTTACTCCTTGCTT |
| | GTCACAGTAGCTTTCATTATCTTTTGGGTGAGGGTGAAATTTAGCCGCT |
| | CTGCGGACGCTCCCGCCTATCAACAGGGCCAGAACCAGCTGTACAACG |
| | AGCTGAACCTGGGCCGCAGAGAAGAGTACGACGTACTCGATAAGAGG |
| | CGCGGCCGCGATCCCGAAATGGGTGGCAAGCCAAGACGCAAAAACCC |
| | TCAGGAGGGCCTGTACAATGAGCTCCAGAAGGACAAGATGGCTGAAG |
| | CCTATAGCGAGATCGGTATGAAAGGTGAGCGCCGGCGCGGTAAGGGC |
| | CACGATGGTCTGTATCAGGGCCTGTCCACGGCTACTAAGGATACCTAC |
| | GACGCACTGCATATGCAGGCCCTGCCTCCGCGGCGCGCAAAGAGGAGT |
| | GGCAGCGGTGCCACGAATTTTTCTCTGCTTAAGCAGGCAGGCGACGTG |
| | GAAGAGAACCCAGGCCCTATGATCGAGACCTACAACCAGACCTCTCCC |
| | AGATCAGCCGCTACGGGGCTGCCCATTAGCATGAAGATTTTCATGTAT |
| | CTCCTGACTGTCTTCCTGATCACGCAAATGATCGGGAGCGCCTTGTTCG |
| | CAGTGTACCTGCACAGGCGCCTGGACAAGATCGAAGATGAGCGGAAC |
| | CTCCACGAGGATTTCGTATTTATGAAGACCATCCAGCGCTGTAACACA |
| | GGGGAACGGTCCCTTAGCCTTCTGAACTGTGAGGAAATCAAGTCCCAG |
| | TTTGAAGGATTTGTGAAGGACATTATGCTGAATAAAGAAGAGACAAAA |
| | AAGGAGAACTCTTTTGAGATGCAGAAGGGCGACCAGAACCCTCAGATC |
| | GCTGCCCATGTGATCTCAGAAGCTTCCTCAAAGACCACGTCTGTGTTGC |
| | AATGGGCCGAAAAGGGTTATTACACAATGAGTAATAACTTGGTCACAC |
| | TTGAGAACGGCAAGCAGCTGACAGTCAAGAGACAGGGCCTGTATTACA |
| | TCTACGCGCAAGTGACTTTCTGTTCCAACAGGGAAGCCTCTAGCCAGG |
| | CCCCGTTCATCGCCTCTCTGTGCCTGAAATCCCCTGGGAGATTTGAACG |
| | TATCCTCCTGCGGGCAGCCAACACTCACAGCTCTGCCAAGCCATGCGG |
| | CCAACAGTCCATCCACCTGGGCGGAGTCTTTGAACTCCAGCCTGGGGC |
| | CTCTGTGTTCGTCAACGTGACCGACCCTAGCCAGGTCTCTCATGGCACC |
| | GGCTTCACTTCCTTCGGACTCCTGAAATTGTGA (SEQ ID NO: 48) |
| CTX_1116 | ATGGCCCTGCCCGTGACCGCCCTGTTGCTGCCGCTGGCTCTCCTGTTGC |
| | ACGCTGCCAGGCCCGAGGTTCAGCTCGTGGAGTCCGGAGGCGGGCTGG |
| | TGCAGCCCGGTCGCTCCCTGCGCCTCTCATGCGCCGCATCTGGTTTTAC |
| | TTTCGATGACTATGCAATGCATTGGGTACGTCAGGCTCCTGGCAAGGG |
| | TCTGGAATGGGTGTCCGGCATCAGCTGGAATTCCGGTAGCACCGGTTA |
| | CGCAGACAGCGTTAAGGGACGGTTTACTATCAGCCGCGATAACGCCAA |
| | GAACTCCCTGTATCTGCAAATGAACTCCCTTCGTGCCGAAGACACCGCT |
| | CTTTATTACTGCGCCAAAGACAGTAGCTCCTGGTACGGTGGAGGCTCC |
| | GCATTCGATATCTGGGGCCAGGGCACGATGGTAACAGTGAGCAGTGGC |
| | GGGGGCGGATCCGGCGGAGGGGGTAGTGGAGGTGGAGGCAGTTCATC |
| | TGAACTCACCCAGGATCCGGCCGTGTCCGTGGCTCTGGGCCAGACTGT |
| | GCGGATCACCTGCCAGGGGGACTCTCTCCGCTCTTATTACGCTTCCTGG |
| | TATCAGCAAAAGCCAGGCCAAGCCCCTGTCCTGGTGATCTATGGTAAG |
| | AATAACCGCCCTAGCGGAATTCCCGATAGGTTCTCCGGATCCAGCTCC |
| | GGCAACACCGCTTCCTTGACCATCACCGGGGCACAGGCCGAGGATGAG |
| | GCAGATTACTATTGTAATAGCAGAGGCTCCAGTGGCAACCACTACGTC |
| | TTTGGGACCGGCACCAAGGTGACCGTTCTCATTGAGGTCATGTATCCCC |
| | CGCCCTACCTTGACAACGAAAAGAGCAACGGAACCATTATCCATGTCA |
| | AAGGCAAACACCTGTGCCCTTCCCCACTGTTCCCTGGCCCTTCCAAGCC |
| | CTTTTGGGTGCTGGTTGTCGTGGGTGGCGTCCTGGCGTGTTACTCCCTC |
| | CTGGTAACAGTTGCGTTTATTATCTTCTGGGTTATGGCTGCCGGAGGCC |
| | CCGGGGCTGGTAGCGCTGCCCCCGTAAGTAGCACATCTAGCCTCCCCC |
| | TGGCTGCCCTGAACATGAGAGTCCGGAGACGCCTTAGTTTGTTTCTCAA |
| | CGTTCGCACTCAGGTGGCTGCCGATTGGACAGCTCTGGCCGAAGAGAT |
| | GGACTTCGAATACCTTGAGATCAGGCAGCTGGAGACTCAGGCTGACCC |
| | CACCGGCAGACTGCTCGATGCCTGGCAGGGCCGCCCGGGGGCGTCCGT |
| | CGGCCGCTTGCTGGAGCTGCTCACCAAGCTGGGGCGCGACGATGTGCT |
| | CCTGGAGCTCGGCCCCTCCATCGAAGAGGACTGTCAGAAGTATATCCT |
| | GAAGCAACAGCAAGAGGAAGCTGAAAAACCACTGCAAGTGGCTGCAG |
| | TGGATTCATCTGTGCCGAGAACCGCCGAGCTGGCTGGTATCACGACCC |
| | TCGATGACCCTCTCGGGCACATGCCAGAGAGGTTTGACGCCTTCATTTG |
| | TTACTGTCCCAGCGATATCCGCGTAAAGTTCTCCCGTAGCGCCGACGCA |
| | CCGGCCTACCAACAGGGCCAGAATCAGCTCTATAACGAACTGAACCTG |
| | GGGCGGAGGGAGGAATACGATGTCCTGGATAAGCGTCGCGGGCGCGA |
| | TCCCGAGATGGGTGGCAAGCCACGTCGCAAGAACCCCCAGGAGGGTCT |
| | CTACAACGAGCTGCAGAAGGACAAAATGGCGGAGGCCTATTCTGAGAT |
| | CGGCATGAAGGGCGAGCGCCGGAGGGGTAAAGGGCACGACGGTCTGT |
| | ACCAGGGCCTGTCTACCGCTACCAAGGATACCTACGATGCCCTGCACA |
| | TGCAAGCCCTCCCTCCCCGCTAA (SEQ ID NO: 49) |
| CTX_1117 | ATGGCCCTGCCCGTGACCGCCCTCCTTCTGCCGCTCGCCCTCTTGCTGC |
| | ATGCCGCTCGCCCAGAGGTCCAGCTGGTCGAGTCTGGGGGAGGTCTGG |
| | TGCAGCCGGGGAGGTCTCTGCGCCTGTCATGTGCCGCTTCAGGCTTTAC |
| | TTTCGATGACTACGCTATGCACTGGGTACGTCAGGCACCCGGCAAGGG |
| | CCTGGAGTGGGTTTCTGGTATCTCTTGGAATAGCGGGTCTACTGGCTAC |
| | GCTGACAGCGTCAAGGGGCGGTTTACTATCAGCCGCGACAATGCTAAG |
| | AACAGCTTGTACCTCCAGATGAACAGTCTGAGGGCCGAGGACACTGCA |
| | CTGTATTACTGCGCTAAGGATTCATCTTCCTGGTATGGTGGCGGTAGCG |
| | CATTTGATATCTGGGGTCAGGGCACAATGGTGACCGTGAGCTCTGGTG |
| | GCGGTGGATCCGGCGGGGGCGGAAGCGGGGGAGGGGGCAGTTCTTCA |
| | GAACTGACACAGGACCCTGCCGTGAGCGTAGCTTTGGGGCAGACTGTC |
| | CGCATCACCTGCCAGGGTGACAGCCTGCGGTCCTACTATGCCAGCTGG |
| | TATCAACAGAAACCTGGGCAGGCTCCCGTGCTGGTGATCTACGGTAAA |
| | AATAACCGCCCGTCCGGGATCCCTGACAGGTTTTCCGGCAGTTCCTCTG |
| | GCAACACGGCTTCTCTGACCATCACTGGAGCCCAGGCGGAAGACGAGG |
| | CTGATTATTACTGCAACTCCCGCGGGTCTAGCGGCAACCACTATGTCTT |
| | CGGCACAGGCACGAAAGTGACTGTGCTTATCGAGGTAATGTATCCTCC |
| | ACCCTACCTGGACAACGAAAAATCCAACGGCACAATTATCCACGTTAA |
| | AGGCAAGCACCTGTGTCCCTCCCCCTTGTTCCCCGGCCCAAGCAAGCCT |
| | TTCTGGGTCTTGGTGGTAGTTGGTGGCGTCCTGGCTTGTTACAGCCTGC |
| | TCGTTACCGTGGCCTTCATTATCTTCTGGGTGATGGCGGCCGGAGGCCC |
| | TGGAGCTGGCTCTGCCGCGCCCGTGTCTTCCACTTCCAGCCTCCCGCTG |
| | GCTGCCCTGAACATGAGAGTGCGTCGCAGACTGTCACTGTTTCTGAAC |
| | GTCCGTACCCAGGTTGCTGCCGACTGGACAGCGCTGGCCGAAGAGATG |
| | GACTTTGAGTACCTGGAAATCCGGCAGCTCGAAACTCAGGCTGATCCC |
| | ACAGGGCGTCTGCTCGACGCGTGGCAGGGAAGGCCAGGCGCCTCTGTG |
| | GGTCGCTTGCTGGAGCTGCTTACTAAACTGGGCAGGGATGACGTTCTTC |
| | TGGAGCTGGGCCCGAGCATTGAGGAAGATTGCCAGAAGTACATTCTCA |
| | AGCAGCAACAGGAGGAAGCCGAAAAGCCCCTGCAGGTGGCCGCTGTG |
| | GATTCCAGCGTGCCGAGGACCGCCGAGCTGGCTGGAATCACTACCCTT |
| | GATGACCCACTCGGTCATATGCCCGAGCGTTTTGATGCGTTCATCTGTT |
| | ATTGTCCGTCCGATATCAAGAAAGTAGCGAAAAAGCCTACCAATAAAG |
| | CTCCCCACCCGAAACAGGAGCCGCAGGAGATCAACTTTCCTGATGACT |
| | TGCCTGGGAGCAACACGGCTGCCCCCGTGCAGGAGACTCTCCACGGTT |
| | GTCAGCCCGTGACTCAGGAAGACGGGAAGGAAAGCCGTATCTCCGTGC |
| | AAGAGCGTCAGCGCGTGAAATTTAGTAGATCTGCCGACGCTCCCGCCT |
| | ACCAACAGGGTCAGAACCAGCTGTACAACGAGCTGAACCTCGGCCGCC |
| | GTGAAGAGTACGATGTTTTGGACAAAAGACGCGGGCGCGATCCTGAGA |
| | TGGGTGGCAAACCTCGTCGCAAGAATCCCCAGGAAGGACTGTATAACG |
| | AGCTGCAGAAGGATAAAATGGCAGAAGCATACAGTGAGATCGGCATG |
| | AAGGGCGAACGCAGGCGCGGGAAGGGTCACGACGGACTGTATCAGGG |
| | GCTGTCAACCGCCACTAAAGATACCTACGATGCTTTGCATATGCAGGC |
| | CCTGCCCCCTAGATAG (SEQ ID NO: 50) |
| CTX_1118 | ATGGCGCTGCCGGTGACCGCCCTGCTCCTGCCTCTGGCTCTGTTGCTCC |
| | ATGCTGCCCGCCCTGAGGTCCAGCTGGTGGAGAGCGGAGGTGGCCTGG |
| | TGCAGCCCGGTCGCAGCCTGCGTCTGAGCTGCGCGGCTTCCGGCTTCAC |
| | CTTCGATGACTATGCCATGCACTGGGTCCGCCAGGCCCCCGGCAAGGG |
| | TCTTGAATGGGTGAGCGGAATCAGTTGGAACAGCGGCTCAACCGGATA |
| | TGCGGATTCTGTGAAGGGGCGCTTTACAATTAGCAGGGACAATGCAAA |
| | GAACAGCCTGTACCTCCAAATGAACTCCCTCCGCGCCGAGGATACAGC |
| | TCTCTATTACTGCGCTAAGGACTCATCCAGCTGGTACGGCGGAGGGAG |
| | CGCTTTCGATATCTGGGGTCAGGGGACCATGGTGACTGTGTCTAGCGG |
| | GGGCGGAGGTTCCGGTGGGGGCGGTTCTGGTGGCGGAGGTTCCAGTAG |
| | CGAGCTCACGCAGGACCCCGCTGTGAGCGTGGCCTTGGGCCAGACCGT |
| | GCGTATCACTTGCCAGGGTGACAGTCTGCGGAGCTATTACGCCTCCTG |
| | GTATCAGCAAAAACCCGGCCAGGCCCCTGTGCTGGTAATCTACGGCAA |
| | AAATAACCGCCCTTCTGGTATCCCCGACCGCTTTAGCGGCTCCAGCTCC |
| | GGCAATACAGCCTCACTGACCATTACCGGAGCTCAGGCCGAGGACGAG |
| | GCCGATTACTATTGCAACTCCAGGGGAAGCTCTGGCAACCATTACGTG |
| | TTCGGGACGGGAACCAAGGTGACAGTTCTTATTGAAGTGATGTACCCG |
| | CCACCCTATCTCGATAATGAGAAATCCAATGGGACGATCATTCACGTC |
| | AAGGGAAAACACCTGTGCCCAAGCCCTTTGTTCCCAGGACCCTCCAAA |
| | CCCTTCTGGGTGCTGGTGGTCGTGGGTGGGGTGCTGGCCTGTTATTCAT |
| | TGCTGGTTACCGTTGCTTTCATTATCTTCTGGGTGATGGCTGCCGGGGG |
| | CCCCGGCGCAGGCAGTGCCGCTCCTGTCTCCAGCACCTCCAGTCTGCCC |
| | TTGGCCGCACTCAACATGCGCGTCCGTAGGCGCCTGAGCCTGTTTCTGA |
| | ACGTACGGACTCAAGTCGCTGCCGACTGGACAGCTCTCGCTGAAGAGA |
| | TGGATTTCGAATACCTCGAGATCCGCCAGCTGGAGACCCAGGCCGACC |
| | CGACCGGCCGTCTCCTGGACGCCTGGCAGGGTAGACCCGGGGCTTCTG |
| | TGGGCAGGTTGCTGGAGCTCCTGACTAAGCTGGGCCGCGATGACGTGT |
| | TGCTGGAACTGGGCCCGTCTATCGAGGAAGACTGCCAGAAGTACATTT |
| | TGAAGCAGCAACAGGAGGAAGCTGAGAAGCCCCTGCAGGTCGCTGCC |
| | GTAGACTCTAGCGTTCCCCGGACCGCCGAACTGGCCGGCATTACCACA |
| | CTGGACGATCCCCTGGGTCACATGCCTGAGAGGTTTGACGCCTTTATCT |
| | GCTACTGTCCCTCTGATATCAAAAAGGTGGCAAAAAAGCCTACTAACA |
| | AAGCTCCACATCCTAAGCAGGAGCCGCAGGAGATCAACTTCCCTGACG |
| | ATCTGCCCGGTTCCAACACTGCCGCTCCGGTGCAGGAGACCCTGCATG |
| | GCTGTCAGCCTGTTACCCAGGAGGACGGGAAGGAATCCCGCATCTCCG |
| | TTCAGGAACGCCAGCGTGTCAAGTTCAGCCGTAGCGCTGATGCCCCTG |
| | CGTATCAACAGGGTCAGAATCAGCTCTACAATGAGTTGAACCTCGGTC |
| | GTCGCGAGGAATACGACGTTCTTGATAAGCGGCGTGGCCGGGACCCTG |
| | AAATGGGAGGTAAGCCCCGCAGAAAGAACCCCCAGGAGGGCCTCTAC |
| | AACGAGCTGCAAAAGGACAAGATGGCAGAGGCCTATTCCGAAATTGG |
| | CATGAAGGGGGAGCGCAGACGCGGCAAAGGCCACGATGGTCTGTATC |
| | AGGGTCTGAGTACCGCAACTAAGGACACCTACGATGCACTGCATATGC |
| | AGGCCCTGCCGCCACGCTAG (SEQ ID NO: 51) |
| CTX_1119 | ATGGCCCTGCCTGTGACCGCCCTGTTGCTGCCTCTCGCTCTCCTGCTCC |
| | ACGCTGCGCGGCCTGAGGTCCAACTTGTGGAAAGCGGCGGAGGGCTGG |
| | TCCAGCCGGGCCGCTCCCTGCGCCTGAGCTGCGCAGCCTCCGGCTTCA |
| | CCTTCGACGATTACGCCATGCATTGGGTGAGGCAGGCCCCAGGCAAGG |
| | GCCTGGAGTGGGTGAGTGGCATCAGTTGGAATTCCGGCTCCACAGGCT |
| | ACGCTGACTCAGTTAAGGGGAGATTCACCATTTCCCGCGATAACGCAA |
| | AGAACAGCCTGTACCTGCAAATGAACTCCCTGCGCGCGGAGGATACCG |
| | CGCTGTACTATTGTGCGAAGGACTCCTCTTCCTGGTATGGAGGCGGTAG |
| | TGCATTCGACATCTGGGGACAGGGAACAATGGTGACTGTGTCCAGCGG |
| | CGGAGGTGGCTCCGGCGGAGGCGGGTCTGGGGGCGGAGGCAGCAGTA |
| | GCGAGTTGACCCAGGATCCTGCTGTGAGTGTGGCGCTGGGTCAGACCG |
| | TGAGGATTACATGCCAGGGGGACTCTCTGCGCTCCTATTACGCCAGTTG |
| | GTACCAACAGAAACCCGGGCAGGCGCCGGTCCTGGTCATCTACGGTAA |
| | AAATAACCGCCCGAGCGGCATTCCCGATAGGTTCAGCGGTAGTTCCTC |
| | TGGGAATACAGCAAGCCTGACTATTACCGGCGCTCAGGCTGAAGACGA |
| | AGCAGACTACTATTGTAACAGCCGCGGCTCCAGCGGCAATCATTATGT |
| | GTTCGGAACCGGTACAAAGGTCACCGTACTGATTGAGGTGATGTATCC |
| | ACCTCCCTATCTGGATAACGAGAAGTCCAACGGCACTATTATCCACGT |
| | GAAGGGCAAGCATCTGTGTCCTAGCCCACTCTTTCCCGGCCCCTCCAAA |
| | CCCTTCTGGGTGCTGGTAGTTGTCGGGGGCGTGCTCGCATGCTATTCCC |
| | TCCTGGTCACCGTGGCTTTCATCATTTTCTGGGTGCGGCTGAAAATCCA |
| | AGTCCGCAAAGCTGCCATCACCTCTTACGAAAAATCTGATGGCGTCTA |
| | CACCGGCCTGTCTACACGCAACCAGGAAACCTACGAGACCCTTAAGCA |
| | CGAAAAGCCGCCTCAGTAA (SEQ ID NO: 52) |
| CTX_1461 | ATGGCCCTGCCTGTGACTGCTTTGCTCCTGCCGTTGGCGCTGCTCCTGC |
| | ACGCTGCCCGTCCTGGTTCCCAGGTGCAGCTGGTCCAAAGCGGCGGGG |
| | GTCTCGTGCAGCCAGGGCGTTCTCTGAGACTGTCTTGCGCTGCCTCTGG |
| | TTTCACTTTCGACGATTATGCTATGCATTGGGTCCGCCAGGCGCCAGGG |
| | AAGGGACTGGAGTGGGTTTCCGGGATCAGCTGGAACTCCGGCTCAATC |
| | GGGTATGCCGACTCAGTTAAGGGCAGGTTCACCATCTCCAGAGACAAC |
| | GCCAAGAATTCACTGTACCTCCAGATGAACAGCCTGAGGGCTGAGGAT |
| | ACCGCCGTGTACTATTGTGCCAAAGATGGATCTTCCTCTTGGAGCTGGG |
| | GCTACTTCGATTACTGGGGCCAAGGGACCTTGGTAACAGTCTCATCCG |
| | GGGGAGGTGGCTCTGGGGGCGGTGGGTCCGGCGGGGGCGGTTCTTCCA |
| | GCGAGCTTACGCAAGACCCAGCCGTGTCTGTGGCACTGGGCCAGACAG |
| | TGCGCACCACTTGCCAGGGAGATGCGCTGCGCAGCTATTACGCCAGCT |
| | GGTACCAACAGAAGCCTGGCCAGGCTCCAATGCTCGTCATTTATGGCA |
| | AGAATAACCGCCCCTCCGGGATCCCAGACCGGTTCAGCGGGAGCGACT |
| | CAGGCGACACCGCTTCCCTGACTATCACAGGCGCCCAGGCTGAGGACG |
| | AGGCGGACTACTATTGCAACAGTCGGGACAGCTCTGGATACCCTGTGT |
| | TTGGCACCGGCACCAAAGTCACCGTGCTCATTGAGGTTATGTACCCTCC |
| | CCCTTATCTCGACAACGAGAAGTCTAACGGAACCATTATCCATGTTAA |
| | GGGCAAGCACCTGTGTCCCAGCCCACTGTTTCCAGGCCCCAGCAAGCC |
| | ATTCTGGGTTCTGGTCGTTGTGGGCGGGGTGCTCGCTTGCTACTCCTTG |
| | CTGGTGACTGTTGCGTTCATTATCTTCTGGGTCAGGCTCAAGATCCAGG |
| | TGCGCAAGGCTGCCATCACTAGTTATGAAAAGTCTGACGGTGTGTACA |
| | CTGGCCTCTCTACGCGCAACCAAGAGACGTATGAGACCCTGAAGCACG |
| | AAAAGCCGCCACAGTAAAAGTTGTCTCCTCCTGCACTGACTGACTGAT |
| | ACAATCGATTTCTGGATCCGCAGGTAAGTGTGCTCGCTTCGGCAGCAC |
| | ATATACTATGAATTCAAAGTCGACTGGAGGCTTGCTTTGGGCTGTATGC |
| | TGTAGATGTTCCGTTCATTAGATCTTTTGGCCTCTGACTGAGATCTATG |
| | ACGGAACATCTACAGGACACAAGGCCCTTTATCAGCACTCACAATGTC |
| | TAGAAAACCTGGAGGCTTGCTTTGGGCTGTATGCTGTGATTGTAGATTA |
| | ATTCCCGGCTTTTGGCCTCTGACTGAGCTGGGGTTATCTACAATCACAG |
| | GACACAAGGCCCTTTATCAGCACTCACATTGCGGCCGCAACCTGGAGG |
| | CTTGCTTTGGGCTGTATGCTGTAAACTCCTGAAACCATGCTCCTTTTGG |
| | CCTCTGACTGAGGGGTAGGTTCAGGAGTTTACAGGACACAAGGCCCTT |
| | TATCAGCACTCACATGGAACAAATGGCCACCGTGGGAGGATGACAACT |
| | CGAGCCTTTTCAATTGTACTAACTTCGAGTCTTCTTTTTTTTTTTCACAG |
| | (SEQ ID NO: 76) |

TABLE 5
Exemplary Anti-Mesothelin Antigen Binding
Domain (ABD) Nucleic Acid Sequences

| ABD ID | Nucleic Acid Sequence |
|---|---|
| M1 | CAAGTGCAGCTACAACAGAGCGGAGCCGAAGTTAAAAAGCCCGGGGCA |
| | TCCGTGAAGGTTTCATGCAAAGCTTCCGGCTATACTTTCACTGGATATTA |
| | CATGCACTGGGTCAGGCAGGCGCCCGGGCAGGGACTGGAGTGGATGGG |
| | CCGGATTAATCCAAATTCTGGAGGCACTAACTACGCCCAGAAATTCCAG |
| | GGCAGAGTGACTATGACCCGGGACACAAGTATAAGCACTGCCTATATGG |
| | AACTGTCGCGCCTGCGGTCCGAAGATACCGCAGTGTACTATTGTGCCAG |
| | GGGGCGGTATTACGGAATGGACGTCTGGGGTCAGGGAACTATGGTTACC |
| | GTGAGCTCCGGCGGAGGGGGTTCTGGCGGGGGCGGCTCCGGCGGTGGC |
| | GGATCCGGCGGGGGAGGCTCTGAAATTGTTCTGACCCAGTCTCCTGCAA |
| | CCCTTAGCCTGAGCCCCGGCGAACGCGCTACGATTTCATGTAGGGCTTCC |
| | CAGTCGGTTAGTAGCAACTTTGCCTGGTATCAGCAGAGGCCAGGTCAGG |
| | CCCCTAGGCTTCTAATTTACGATGCGAGCAACCGAGCTACGGGCATTCC |
| | ACCAAGATTCTCTGGTAGCGGAAGTGGCACCGATTTTACACTGACTATTT |
| | CATCACTCGAACCCGAGGATTTTGCAGCCTATTACTGCCACCAGAGGTCT |
| | AACTGGCTGTACACTTTCGGCCAGGGAACTAAGGTGGACATAAAA (SEQ |
| | ID NO: 53) |
| M2 | CAAGTGCAGCTGGTTCAGTCGGGTGCCGAAGTGAAAAAACCCGGCGCTT |
| | CAGTGAAAGTGTCCTGTAAGGCCTCCGGATATACCTTTACTGGTTACTAT |
| | ATGCACTGGGTACGGCAGGCCCCAGGTCAAGGCCTGGAATGGATGGGAT |
| | GGATTAACCCTAACAGCGGCGGGACAAATTATGCTCAGAAGTTTCAAGG |
| | AAGAGTGACGATGACTCGCGACACCTCTATAAGTACCGCATACATGGAA |
| | CTATCAAGGCTCCGTTCCGACGATACTGCCGTGTACTACTGCGCCAGGG |
| | ACTTGAGAAGGACCGTTGTGACACCTCGGGCCTATTATGGTATGGACGT |
| | GTGGGGCCAGGGCACCACAGTGACCGTCTCTAGCGGGGGCGGCGGATCC |
| | GGCGGCGGGGGCAGCGGAGGGGGTGGGTCTGGGGGAGGTGGGTCCGAT |
| | ATCCAGCTCACCCAGTCTCCATCTACCTTAAGTGCCTCTGTCGGGGATCG |
| | CGTAACAATCACTTGCCAGGCCAGCCAGGACATTAGCAATTCCTTAAAC |
| | TGGTATCAGCAGAAGGCCGGGAAGGCGCCTAAGCTTCTAATTTATGATG |
| | CCAGCACTCTCGAAACCGGGGTGCCGAGTAGATTCTCCGGATCCGGCAG |
| | CGGGACCGACTTCTCTTTCACTATTAGTTCGTTGCAGCCCGAAGATATTG |
| | CCACCTATTACTGTCAACAGCACGACAACTTGCCTCTCACGTTCGGACAG |
| | GGAACCAAGGTTGAAATTAAA (SEQ ID NO: 54) |
| M3 | CAGGTTCAGCTGGTCCAGAGCGGAGCCGAGGTGAAAAAGCCTGGCGCG |
| | CCAGTCAAAGTCTCATGTAAAGCTTCCGGATATACCTTCACCGGGTACTA |
| | TATGCACTGGGTGAGACAGGCCCCAGGGCAAGGCTTAGAGTGGATGGG |
| | CTGGATCAACCCAAACTCGGGGGGAACAAACTATGCCCAAAAATTCCAA |
| | GGCCGTGTCACAATGACACGTGACACTTCAATATCAACCGCGTACATGG |
| | AACTTTCACGGCTACGTTCCGATGACACCGCAGTATACTACTGCGCAAG |
| | AGGGGAATGGGACGGCTCCTATTATTACGATTACTGGGGACAAGGCACG |
| | CTGGTGACTGTATCTAGCGGCGGGGGAGGTAGCGGCGGTGGCGGGAGT |
| | GGCGGTGGCGGCTCCGGAGGAGGGGGAAGCGACATCGTTCTGACCCAG |
| | ACACCTTCTTCGTTGAGTGCTTCTGTCGGAGACAGGGTTACGATTACCTG |
| | TCGGGCGTCACAGTCAATTAACACCTATCTGAACTGGTATCAGCATAAG |
| | CCTGGCAAGGCTCCAAAGCTGTTGATCTATGCAGCCAGTAGTCTTCAATC |
| | CGGCGTGCCATCCCGGTTCTCAGGGTCAGGTAGCGGTACTGATTTTACTC |
| | TTACTATCAGCTCCCTCCAGCCTGAGGACTTTGCCACATATTACTGTCAG |
| | CAGAGCTTTAGCCCTCTTACCTTCGGGGGGGGCACCAAACTCGAGATTA |
| | AG (SEQ ID NO: 55) |
| M4 | CAGGTGCAGCTGGTGGAATCAGGTGGGGGTCTAGTGCAGCCAGGAGGCT |
| | CACTGAGATTAAGTTGCGCTGCTAGTGGCTTCACCTTTAGCTCCTACTGG |
| | ATGCACTGGGTGAGACAGGTCCCCGGCAAGGGCTTAGTGTGGGTGTCAA |
| | GAATCAACACAGATGGATCTACAACAACATACGCAGACTCCGTCGAAGG |
| | GCGCTTCACAATCTCTCGTGACAACGCAAAAAACACGCTTTATCTCCAG |
| | ATGAACTCACTGCGAGACGACGATACCGCCGTCTACTACTGCGTGGGAG |
| | GCCATTGGGCAGTTTGGGGGCAGGGGACAACTGTCACTGTCTCTTCTGG |
| | AGGCGGAGGCTCAGGGGGCGGCGGCAGTGGCGGTGGGGGGAGTGGAGG |
| | CGGCGGATCAGATATCCAAATGACACAATCCCCAAGTACCTTAAGTGCC |
| | AGTGTCGGCGACCGGGTGACCATTACATGCCGAGCCTCCCAGTCTATTA |
| | GCGATCGTTTAGCATGGTATCAGCAGAAGCCGGGGAAGGCACCGAAGCT |
| | GCTCATTTATAAGGCTAGTAGCTTAGAATCTGGGGTTCCATCCAGATTTT |
| | CAGGTTCTGGATCAGGAACCGAGTTCACTCTTACGATCTCCTCCCTGCAG |
| | CCAGACGACTTCGCTGTTTACTATTGCCAACAGTACGGTCATTTGCCTAT |
| | GTACACATTTGGGCAGGGCACCAAGGTCGAAATCAAG (SEQ ID NO: 56) |
| M5 | CAAGTACAACTCGTTCAGAGCGGGGCGGAAGTCGAGAAACCAGGGGCC |
| | TCCGTTAAAGTTAGCTGCAAGGCAAGTGGATACACCTTCACCGACTATT |
| | ACATGCATTGGGTTCGACAGGCTCCCGGCCAAGGTCTCGAATGGATGGG |
| | ATGGATCAATCCAAACAGCGGGGGCACCAACTACGCTCAGAAATTTCAG |
| | GGACGCGTGACAATGACTAGAGACACGTCCATTTCCACGGCCTACATGG |
| | AGCTAAGTCGACTCAGGTCCGACGATACAGCCGTTTATTATTGCGCCTCT |
| | GGTTGGGACTTCGATTATTGGGGGCAGGGTACCCTAGTGACTGTGAGTT |
| | CCGGAGGGGGGGGATCTGGCGGGGGCGGTAGCGGTGGCGGGGGGTCGG |
| | GCGGCGGTGGCAGTGATATCGTTATGACTCAGTCACCTTCCAGCCTTAGC |
| | GCAAGCGTGGGTGACCGCGTGACAATTACCTGCCGGGCATCCCAGTCCA |
| | TCAGATATTACTTAAGTTGGTACCAGCAGAAGCCGGGGAAGGCCCCAAA |
| | ACTGCTGATCTACACCGCCAGTATCCTGCAGAATGGGGTGCCATCCAGA |
| | TTTAGTGGCTCCGGATCCGGCACTGATTTCACTCTGACGATTAGCTCTCT |
| | GCAACCTGAGGATTTTGCAACCTATTACTGCCTGCAGACCTATACCACCC |
| | CCGATTTTGGGCCCGGGACCAAAGTGGAGATCAAA (SEQ ID NO: 57) |
| M6 | CAGGTGCAACTCGTGCAATCGGGAGCTGAAGTGAAGAAGCCAGGGGCC |
| | AGCGTTAAAGTGTCCTGTAAGGCAAGTGGTTACACATTTACAAGCTACT |
| | ATATGCATTGGGTCCGACAGGCTCCTGGACAGGGGTTGGAGTGGATGGG |
| | AATCATCAATCCGAGTGGCGGCAGCACTTCCTATGCCCAAAAGTTCCAG |
| | GGCAGGGTGACAATGACCCGGGACACGAGTACTAGTACAGTCTATATGG |
| | AGCTGAGCTCCCTCAGATCAGAGGATACCGCAGTGTACTATTGTGCACG |
| | GTATCGGCTGATCGCCGTTGCCGGTGACTATTACTATTACGGCATGGATG |
| | TGTGGGGCCAGGGGACGATGGTGACCGTGTCTAGCGGCGGCGGGGGGA |
| | GCGGTGGGGGAGGGTCCGGCGGCGGTGGAAGCGGCGGGGGCGGATCAG |
| | ACATCCAAATGACACAGTCACCCTCCTCGGTGAGTGCCTCCGTGGGAGA |
| | CCGTGTGACTATCACTTGTCGCGCCAGCCAGGGGGTGGGTCGATGGCTG |
| | GCGTGGTATCAGCAGAAACCAGGCACCGCTCCTAAGCTTCTTATCTACG |
| | CAGCATCGACTCTCCAGAGCGGCGTCCCAAGCCGGTTCAGTGGCTCCGG |
| | GAGCGGGACCGACTTTACCCTTACCATAAACAATCTCCAGCCAGAAGAC |
| | TTCGCAACCTACTATTGCCAGCAGGCTAATTCCTTTCCGTTGACTTTTGG |
| | CGGGGGGACACGCCTGGAGATTAAG (SEQ ID NO: 58) |
| M7 | CAGGTGCAACTGGTCCAGAGCGGAGGAGGCGTGGTTCAGCCTGGAAGG |
| | TCTCTGCGATTGTCTTGCGCAGCGAGCGGCTTCACCTTCTCTTCATACGC |
| | AATGCACTGGGTTCGCCAGGCACCCGGCAAGGGGCTGGAGTGGGTCGCA |
| | GTAATTAGTTATGACGGCTCTAATAAATATTATGCTGACTCTGTTAAGGG |
| | CAGATTCACAATCTCTCGCGACAACAGTAAAAACACACTATATCTGCAG |
| | ATGAACAGCTTGCGTGCCGAAGACACAGCGGTTTACTATTGTGCCAGAT |
| | GGAAGGTTTCTAGCTCCTCTCCAGCCTTCGATTATTGGGGTCAGGGCACA |
| | CTGGTCACGGTCTCAAGCGGTGGGGGTGGCTCAGGCGGCGGCGGGTCCG |
| | GTGGCGGCGGATCAGGAGGGGGCGGGAGCGAAATTGTGTTGACCCAAA |
| | GTCCAGCCACCCTGTCCCTGTCCCCAGGAGAGCGAGCGATCTTAAGCTG |
| | TAGAGCTTCTCAATCCGTCTACACCAAGTACTTAGGCTGGTATCAGCAG |
| | AAACCTGGTCAGGCTCCTAGGTTGCTGATTTACGACGCCAGTACGCGCG |
| | CAACGGGCATTCCCGACCGGTTTAGCGGCAGCGGTAGCGGAACTGATTT |
| | TACCCTCACCATCAACCGTTTGGAACCAGAAGACTTCGCCGTGTACTATT |
| | GCCAGCACTATGGCGGGTCTCCCTTAATCACCTTCGGCCAGGGGACCCG |
| | GCTGGAAATCAAG (SEQ ID NO: 59) |
| M8 | CAGGTGCAGCTGCAGCAGTCAGGCGCAGAGGTGAAGAAACCCGGAGCT |
| | AGCGTTAAGGTAAGCTGCAAGACCTCCGGGTACCCCTTTACAGGGTACA |
| | GTCTGCACTGGGTTCGACAGGCACCAGGGCAGGGCTTGGAATGGATGGG |
| | GTGGATCAATCCTAACTCAGGCGGCACCAACTACGCCCAAAAGTTCCAG |
| | GGGAGAGTGACAATGACCAGGGATACAAGCATTAGCACTGCATATATG |
| | GAGCTATCACGGCTGCGGTCCGATGACACTGCGGTGTACTACTGCGCTC |
| | GTGACCACTATGGAGGCAACAGCTTGTTCTACTGGGGTCAGGGAACCCT |
| | CGTGACAGTTAGCTCCGGAGGGGGCGGGTCTGGCGGCGGGGGATCTGG |
| | AGGTGGTGGATCCGGAGGTGGCGGAAGCGACATTCAGCTCACGCAGTCG |
| | CCCAGCTCCATCAGCGCCTCTGTTGGAGACACCGTGAGTATTACCTGTAG |
| | AGCAAGCCAAGACAGTGGCACCTGGTTGGCGTGGTACCAGCAAAAACCT |
| | GGTAAGGCTCCTAATTTACTCATGTACGATGCTTCCACACTGGAGGACG |
| | GAGTGCCGTCCCGGTTTAGCGGGAGCGCCTCAGGAACAGAATTTACATT |
| | AACCGTCAATAGGCTGCAGCCCGAAGACTCAGCAACTTACTATTGTCAA |
| | CAGTATAACAGCTATCCTCTAACTTTTGGAGGTGGCACAAAGGTAGATA |
| | TAAAG (SEQ ID NO: 60) |
| M9 | CAGGTGCAGCTAGTGCAATCCGGCGCTGAGGTTAAAAAGCCAGGTGCCA |
| | GCGTTGAGGTGAGCTGTAAGGCTAGCGGCTACACTTTTACTTCATATTAC |
| | ATGCACTGGGTTCGTCAAGCCCCTGGCCAGGGACTGGAGTGGATGGGAA |
| | TTATCAACCCTTCCGGGGGCTCTACTGGCTACGCACAAAAATTCCAGGG |
| | CCGCGTGACCATGACACGGGACACTTCAACCAGCACCGTGCATATGGAG |
| | CTGAGCTCACTCAGGAGCGAAGACACCGCTGTGTACTATTGTGCTAGAG |
| | GCGGCTACTCTAGCTCCTCTGACGCCTTCGACATCTGGGGTCAGGGTACT |
| | ATGGTGACAGTAAGCAGCGGCGGCGGTGGGTCAGGAGGGGGGGGATCT |
| | GGAGGCGGTGGCAGCGGTGGCGGAGGGTCCGACATTCAGATGACTCAG |
| | TCACCTCCCTCCTTGAGCGCTTCTGTGGGCGATAGGGTAACCATCACATG |
| | CCGAGCAAGCCAGGACATCAGTTCCGCACTTGCGTGGTACCAGCAGAAG |
| | CCGGGCACTCCTCCCAAGCTCCTAATTTATGATGCTTCTTCATTAGAATC |
| | AGGCGTTCCTTCACGCTTCAGCGGTAGCGGCTCAGGTACCGATTTCACTC |
| | TCACCATTTCTAGTCTACAACCAGAAGATTTTGCAACTTATTATTGCCAG |
| | CAGTTTAGCTCTTATCCTTTAACCTTCGGTGGAGGTACTCGACTCGAGAT |
| | AAAA (SEQ ID NO: 61) |
| M10 | CAGGTCCAGCTGGTCCAAAGCGGTGCAGAAGTTAAGAAACCTGGTGCCT |
| | CCGTTAAAGTATCTTGCAAAGCCAGCGGATACACTTTTACGTCATATGG |
| | GATCTCTTGGGTTAGACAGGCTCCAGGGCAGGGGTTGGAATGGATGGGT |
| | TGGATCTCAGCGTATAACGGGAACACTAATTACGCGCAGAAGCTCCAGG |
| | GCAGGGTCACTATGACTACTGATACAAGCACAAGCACCGCTTACATGGA |
| | ACTAAGAAGCCTGCGGTCCGATGACACGGCAGTTTATTACTGCGCAAGG |
| | GTTGCAGGCGGAATTTACTATTACTACGGCATGGATGTTTGGGGCCAAG |
| | GGACTACTATTACCGTTAGCAGCGGAGGAGGCGGGTCAGGGGGAGGCG |
| | GTTCGGGCGGCGGAGGCAGTGGGGGCGGCGGTTCCGATATTGTCATGAC |
| | ACAGACACCGGACTCCTTGGCCGTTTCATTGGGCGAACGCGCGACTATC |
| | AGCTGTAAGAGCAGTCATAGCGTTCTCTACAACAGGAACAACAAGAACT |
| | ACCTCGCGTGGTATCAGCAGAAACCTGGCCAACCACCTAAACTGCTGTT |
| | TTACTGGGCATCCACAAGAAAGTCGGGTGTGCCAGACCGATTCTCGGGT |
| | TCTGGGTCCGGCACGGACTTCACGCTGACAATAAGCTCACTCCAACCTG |
| | AAGATTTCGCAACCTATTTCTGTCAGCAGACCCAGACTTTCCCCCTGACA |
| | TTTGGGCAGGGCACGAGGTTGGAGATCAAT (SEQ ID NO: 62) |
| M11 | CAGGTCCAGCTGCAGCAGTCCGGAGCAGAAGTCAAGAAGCCTGGCGCCT |
| | CAGTCAAAGTGTCATGCAAGGCCAGCGGCTATACATTCACCGGATACTA |
| | CATGCATTGGGTCAGGCAGGCACCTGGGCAGGGGCTGGAGTGGATGGG |
| | CTGGATTAATCCTAACTCAGGGGGCACCAACTATGCCCAGAACTTTCAG |
| | GGACGCGTGACAATGACTCGAGATACTTCAATTTCTACAGCCTACATGG |
| | AGCTCCGGAGGCTGAGATCCGACGACACAGCGGTGTACTACTGCGCCAG |
| | TGGATGGGACTTTGACTATTGGGGGCAGGGTACTCTGGTGACGGTGTCC |
| | TCTGGGGGTGGCGGCAGCGGCGGGGGGGGCAGCGGAGGCGGGGGGAGT |
| | GGGGGCGGAGGATCGGACATTAGGATGACACAGAGCCCAAGCTCCCTCT |
| | CTGCCAGCGTAGGCGACAGGGTCACTATTACATGCCGTGCCTCCCAGTC |
| | TATTAGGTACTATCTGTCCTGGTATCAGCAGAAACCTGGAAAAGCGCCC |
| | AAGCTCCTGATCTACACAGCCTCGATCCTCCAAAATGGGGTGCCATCGA |
| | GATTTAGTGGGTCCGGTTCTGGCACTGACTTTACCCTGACTATCTCGTCA |
| | CTTCAGCCTGAGGACTTTGCGACTTATTACTGCCTTCAGACATATACTAC |
| | TCCCGACTTCGGTCCAGGCACCAAGGTAGAGATAAAG (SEQ ID NO: 63) |
| M12 | CAGGTGCAATTGGTTCAGAGCGGGGCCGAAGTCAAGAAGCCTGGGGCCT |
| | CCGTGAAGGTTAGCTGCAAAGCCAGCGGATACACCTTTACAGGCTACTA |
| | TATGCACTGGGTTCGACAGGCCCCCGGTCAGGGACTTGAATGGATGGGA |
| | AGGATCAACCCCAACAGCGGCGGAACTAATTACGCCCAGAAATTCCAGG |
| | GGAGGGTGACAATGACCACCGACACCTCAACTTCCACAGCATACATGGA |
| | GTTGAGATCCCTGAGATCTGACGACACCGCAGTTTATTACTGTGCTAGA |
| | ACCACAACCTCCTACGCCTTCGACATTTGGGGTCAAGGAACCATGGTGA |
| | CCGTGTCTTCTGGCGGTGGCGGGAGCGGCGGAGGTGGGTCAGGAGGAG |
| | GGGGAAGTGGAGGGGGCGGTTCAGATATTCAGCTCACACAATCCCCCTC |
| | TACATTGAGCGCATCTGTAGGCGACCGTGTTACAATAACGTGTCGCGCG |
| | TCCCAATCCATCAGTACATGGTTGGCCTGGTACCAGCAGAAGCCGGGTA |
| | AAGCTCCTAATTTGCTGATCTATAAGGCCTCCACTCTGGAGAGCGGCGT |
| | GCCTAGCCGGTTTAGTGGGTCTGGATCGGGGACCGAGTTTACCTTAACT |
| | ATCAGTAGCCTACAGCCAGACGATTTTGCTACATACTACTGCCAGCAGT |
| | ACAATACATACAGCCCTTACACCTTCGGCCAAGGCACTAAGCTGGAGAT |
| | AAAG (SEQ ID NO: 64) |
| M13 | CAAGTGCAGCTGGTTCAGTCAGGCGGTGGGCTCGTGAAACCAGGGGGCA |
| | GTCTACGTCTGAGTTGCGAGGCAAGCGGCTTTATCTTTTCCGATTATTAC |
| | ATGGGTTGGATCAGACAGGCCCCAGGCAAAGGGCTCGAGTGGGTGAGC |
| | TACATCGGCCGTTCAGGTAGTTCTATGTACTATGCTGACAGTGTGAAAG |
| | GGCGGTTTACTTTTAGCCGGGACAACGCAAAGAACTCTCTGTACCTGCA |
| | AATGAACTCGTTGCGAGCGGAGGACACAGCTGTATACTATTGCGCCGCA |
| | TCACCTGTTGTGGCCGCAACGGAAGACTTCCAGCACTGGGGGCAGGGAA |
| | CCCTTGTCACGGTATCTAGCGGGGGTGGTGGCTCAGGCGGGGGCGGCAG |
| | TGGCGGCGGAGGGTCAGGGGGCGGGGGATCCGACATTGTGATGACACA |
| | AACTCCCGCGACTTTGAGTCTGAGCCCAGGAGAACGTGCGACCCTCTCC |
| | TGTAGGGCCTCACAGAGTGTGACCAGTAATTACCTAGCCTGGTATCAGC |
| | AGAAGCCGGGACAAGCTCCGCGTTTGCTGCTATTTGGCGCGTCCACTCG |
| | GGCAACCGGCATCCCAGACCGGTTCTCAGGATCCGGTAGTGGAACTGAT |
| | TTCACGCTGACCATTAATCGGCTCGAACCCGAGGATTTCGCCATGTATTA |
| | TTGTCAGCAGTACGGCTCTGCACCCGTTACATTTGGCCAAGGAACCAAA |
| | CTTGAAATTAAG (SEQ ID NO: 65) |
| M14 | CAGGTACAGTTGGTGCAGTCCGGCGCAGAGGTAAGGGCCCCTGGCGCTT |
| | CTGTAAAGATCAGCTGCAAGGCTTCAGGATTTACATTCCGGGGATACTA |
| | CATTCACTGGGTGCGCCAGGCTCCTGGCCAAGGCTTGGAGTGGATGGGC |
| | ATCATCAATCCCAGCGGCGGATCTCGCGCCTACGCACAAAAGTTCCAAG |
| | GCCGCGTGACCATGACCCGTGACACATCAACTTCCACAGTCTATATGGA |
| | ATTAAGCAGTCTCCGGTCAGACGACACCGCAATGTACTACTGTGCCCGA |
| | ACCGCCTCCTGCGGTGGAGATTGTTATTATCTGGATTATTGGGGGCAAG |
| | GCACCTTGGTGACCGTTTCCTCAGGGGGCGGCGGATCGGGGGGGGGGGG |
| | TTCCGGGGGAGGAGGAAGCGGCGGCGGTGGCTCTGATATCCAGATGACT |
| | CAGAGTCCGCCCACTCTGAGCGCGTCCGTTGGTGATCGCGTTACCATTAC |
| | ATGCCGTGCCTCTGAGAACGTGAACATTTGGTTGGCTTGGTACCAGCAG |
| | AAGCCCGGAAAAGCCCCCAAATTGCTGATCTACAAAAGCAGCAGCCTGG |
| | CCTCAGGAGTACCAAGCAGATTTTCCGGCTCCGGAAGCGGAGCAGAATT |
| | TACCCTGACAATCTCCAGCCTGCAGCCCGACGATTTTGCCACATATTACT |
| | GCCAGCAGTACCAGTCCTATCCACTCACTTTTGGCGGTGGGACTAAAGT |
| | GGATATTAAA (SEQ ID NO: 66) |
| M15 | CAGGTGCAGTTGGTGCAATCTGGCGGAGGCTTGGTCCAGCCAGGTAGAA |
| | GTTTGAGGCTCTCCTGTGCCGCTTCCGGCTTTACCTTCGATGACTACGCT |
| | ATGCACTGGGTGAGACAGGCCCCGGGAAAAGGTCTGGAATGGGTCTCCG |
| | GGATCAGCTGGAACAGTGGCTCTATCGGTTACGCGGACAGTGTAAAGGG |
| | GAGGTTCACCATTAGTCGCGATAACGCCAAGAATAGCCTCTATCTGCAG |
| | ATGAACTCCCTGAGGGCCGAAGACACAGCAGTGTATTACTGCGCGAAGG |
| | ATGGCAGCAGCAGCTGGAGTTGGGGGTACTTCGACTACTGGGGCCAGGG |
| | CACTTTGGTCACAGTCTCCAGCGGAGGAGGGGGAAGTGGCGGTGGAGG |
| | AAGCGGCGGCGGCGGTTCTTCGAGCGAACTGACGCAGGATCCTGCAGTG |
| | TCTGTAGCCCTGGGCCAGACCGTTCGGACCACGTGCCAAGGCGATGCGC |
| | TCAGATCTTATTACGCATCATGGTACCAGCAAAAGCCAGGACAGGCTCC |
| | TATGTTGGTGATCTACGGGAAGAATAACCGCCCATCCGGGATCCCAGAT |
| | CGCTTCTCCGGTAGCGACTCGGGAGATACAGCCTCCCTGACCATAACCG |
| | GCGCCCAGGCAGAGGACGAAGCTGATTACTACTGTAATTCTCGCGACTC |
| | GTCCGGATACCCCGTTTTCGGTACGGGCACCAAGGTCACAGTGCTC (SEQ |
| | ID NO: 67) |
| M16 | GAGGTGCAGTTGGTCGAATCTGGCGGCGGGCTAGTTCAGCCTGGGAGAA |
| | GCCTTCGACTTAGCTGCGCCGCTAGTGGCTTTACATTCGACGACTACGCC |
| | ATGCATTGGGTTAGACAAGCTCCCGGAAAGGGTCTTGAATGGGTCTCCG |
| | GAATATCTTGGAATTCGGGATCCACAGGTTACGCAGATTCAGTCAAAGG |
| | TCGGTTTACTATATCCCGGGACAACGCAAAGAATAGCCTGTACCTTCAG |
| | ATGAACTCACTGAGAGCGGAGGACACTGCTTTGTACTACTGTGCCAAAG |
| | ATTCCTCATCTTGGTATGGAGGAGGAAGCGCGTTTGATATATGGGGCCA |
| | GGGAACCATGGTCACGGTATCTTCTGGCGGCGGCGGTAGCGGAGGCGGC |
| | GGCTCCGGCGGAGGAGGATCGTCAAGTGAGCTGACACAAGAGCCCGCA |
| | GTGTCAGTGGCCCTTGGACAGACTGTCAGGATCACTTGCCAGGGAGATT |
| | CACTGAGATCTTATTATGCAAGCTGGTACCAGCAAAAACCAGGTCAGGC |
| | ACCAGTACTGGTTATTTTCGGGCGGAGCAGGCGCCCTTCAGGCATTCCTG |
| | ACCGTTTTTCCGGGTCCAGCTCCGGTAACACAGCTAGTCTGATCATCACG |
| | GGAGCCCAAGCTGAAGATGAAGCCGATTACTACTGCAACTCGCGCGACA |
| | ATACCGCTAATCACTATGTGTTCGGGACCGGCACCAAATTAACTGTCCTG |
| | (SEQ ID NO: 68) |
| M17 | GAAGTGCAGCTGGTTGAGTCTGGAGGCGGGCTGGTACAACCCGGCAGGT |
| | CCCTCAGACTTTCATGCGCAGCCAGTGGATTCACCTTTGACGACTACGCC |
| | ATGCATTGGGTAAGGCAGGCTCCAGGCAAAGGTCTGGAGTGGGTGTCAG |
| | GCATATCGTGGAATAGCGGGAGCACCGGCTATGCAGACAGTGTTAAGGG |
| | AAGGTTTACCATATCACGGGATAATGCTAAAAATTCATTGTATTTACAG |
| | ATGAACTCCCTGAGGGCCGAGGATACCGCCTTATATTACTGCGCAAAGG |
| | ACAGTTCCTCCTGGTATGGTGGGGGGAGCGCATTCGATATCTGGGGCCA |
| | AGGCACTATGGTGACAGTGTCCTCCGGGGGAGGGGGTTCAGGAGGGGG |
| | CGGGAGCGGTGGGGGGGGTCCAGCAGTGAACTTACTCAGGACCCCGCC |
| | GTGTCTGTTGCTCTAGGCCAAACGGTGAGAATAACCTGTCAAGGCGATT |
| | CCTTGAGGTCATACTACGCCAGCTGGTACCAGCAGAAACCTGGGCAGGC |
| | CCCTGTTCTAGTGATATACGGGAAAAATAACCGTCCTTCCGGAATCCCTG |
| | ATCGCTTTTCTGGGTCTTCATCTGGTAACACTGCCTCTCTCACAATTACC |
| | GGAGCACAGGCAGAGGATGAGGCCGACTACTACTGCAATTCTCGGGGAT |
| | CCTCTGGCAACCATTATGTCTTCGGCACAGGCACAAAAGTGACTGTGCTT |
| | (SEQ ID NO: 69) |
| M18 | CAGGTGCAGCTGGTGCAGTCAGGTGGGGGTTTAGTGCAGCCCGGAGGCT |
| | CCCTGCGGCTCTCCTGTGCGGCCTCAGGATTCACTTTTTCCTCTTACTGG |
| | ATGCACTGGGTGCGGCAAGCTCCCGGCAAGGGGCTGGTATGGGTCAGCC |
| | GGATCAATTCAGACGGGTCCAGCACGAGCTACGCCGACTCAGTCAAGGG |
| | AAGATTTACCATCAGCCGAGATAACGCAAAAAACACTCTTTACCTGCAA |
| | ATGAATTCATTACGCGCCGAGGATACGGCCGTATACTATTGCGTGAGGA |
| | CAGGCTGGGTTGGTAGTTACTACTACTATATGGACGTATGGGGCAAGGG |
| | TACCACCGTTACAGTGAGCTCAGGAGGTGGAGGCTCTGGAGGTGGAGGC |
| | AGCGGCGGAGGAGGATCAGGCGGAGGTGGCTCCGAGATTGTCTTGACCC |
| | AGTCTCCCGGGACTTTGTCACTGTCTCCCGGTGAAAGGGCCACCCTGTCC |
| | TGCAGGGCTAGTCAATCCGTGAGTAGTAATTACCTGGCATGGTATCAGC |
| | AAAAGCCTGGCCAGCCACCAAGGCTGCTCATTTATGATGTGTCGACACG |
| | AGCCACAGGGATACCCGCACGATTCTCAGGCGGTGGCTCCGGGACCGAC |
| | TTTACTCTGACCATCAGCAGTCTAGAGCCAGAGGATTTCGCTGTGTACTA |
| | CTGCCAACAGCGCAGTAACTGGCCTCCTTGGACGTTCGGTCAGGGAACT |
| | AAGGTGGAAATTAAG (SEQ ID NO: 70) |
| M19 | CAGGTCCAGCTAGTCCAGTCGGGCGGAGGAGTCGTCCAACCTGGGAGAA |
| | GTTTACGACTGTCCTGCGCGGCCTCAGGGTTCACCTTCTCTAGTTACGGC |
| | ATGCACTGGGTAAGACAGGCACCTGGGAAGGGCCTTGAGTGGGTGGCG |
| | GTCATCTCCTATGATGGCAGTAACAAATATTACGCTGACTCTGTGAAAG |
| | GTAGGTTTACTATTTCCAGAGATAATTCCAAAAACACTCTTTATCTACAG |
| | ATGAACAGCCTCAGAGCAGAAGATACCGCCGTATATTATTGCGCAAAGG |
| | GCTACTCAAGGTACTACTACTATGGGATGGACGTGTGGGGACAGGGGAC |
| | GACAGTGACCGTTTCAAGTGGAGGCGGTGGGTCTGGGGGCGGCGGGAG |
| | CGGCGGGGGCGGGTCCGGCGGTGGCGGCAGCGAGATCGTGATGACTCA |
| | GTCACCTGCAACCTTGTCATTGAGCCCTGGGGAGCGAGCAATTCTGTCCT |
| | GCAGAGCCTCCCAGAGCGTCTACACTAAGTACCTGGGATGGTACCAGCA |
| | GAAACCCGGCCAGGCTCCTCGCTTACTGATTTACGACGCTTCCACTCGAG |
| | CAACTGGCATACCCGATAGATTCTCAGGAAGTGGGAGCGGAACTGATTT |
| | CACCCTGACAATCAATAGACTTGAGCCTGAGGACTTTGCTGTATATTACT |
| | GCCAGCATTACGGCGGGTCCCCGCTGATCACCTTTGGCCAGGGTACAAA |
| | GGTCGACATTAAA (SEQ ID NO: 71) |
| M20 | CAAGTGCAGCTCGTCCAGTCAGGGGGGGGCTGGTTCAACCGGGGGGTA |
| | GCTTGCGTCTGTCCTGTGCTGCTTCCGGTTTTACATTCAGCTCCTATGCCA |
| | TGTCATGGGTACGACAAGCTCCCGGCAAGGGACTCGAATGGGTAAGTGC |
| | AATATCTGGATCTGGGGGCTCTACCTACTACGCCGATTCCGTTAAGGGCC |
| | GGTTCACAATCAGTCGGGACAATAGTAAGAATACACTGTACCTGCAGAT |
| | GAACAGCCTGCGAGCTGAGGATACAGCGGTGTATTACTGTGCTAAACGT |
| | GAGGCCGCGGCAGGACATGACTGGTACTTTGACTTATGGGGCCGAGGAA |
| | CACTGGTGACCGTCTCTTCAGGAGGGGGCGGAAGCGGAGGAGGTGGAA |
| | GTGGCGGGGGAGGATCCGGAGGCGGAGGCTCTGATATTAGGGTTACCCA |
| | GTCCCCCAGCTCACTGAGCGCGTCAGTGGGCGATCGCGTCACGATAACT |
| | TGTCGTGCTTCCCAGAGCATTAGTAGCTACCTTAACTGGTACCAGCAAA |
| | AGCCTGGGAAGGCACCCAAATTGTTGATTTATGCCGCCTCCTCCCTACAA |
| | TCCGGGGTTCCTTCTAGGTTTAGCGGTTCTGGTTCGGGGACCGACTTCAC |
| | TCTGACCATATCAAGTCTTCAGCCCGAGGACTTCGCCACCTATTATTGCC |
| | AGCAGTCCTATTCAATCCCACTGACATTCGGCCAGGGCACCAAGGTGGA |
| | AATAAAA (SEQ ID NO: 72) |

TABLE 6a
shRNA Guide Strand Sequences

| | Target | Scaffold | Guide Sequence | SEQ ID |
|---|---|---|---|---|
| Name | Gene | Origin | (5′ to 3′) | NO: |
| hu_shSIRPA_583.2 | SIRPα | miR-155 | AUUGUAGAUUAAUUCCCGGCC | 77 |
| hu_shSIRPA_584.2 | SIRPα | miR-155 | UGAUUGUAGAUUAAUUCCCGGC | 78 |
| hu_shSIRPA_1301.2 | SIRPα | miR-155 | AUACAUUCACCAGGAGCCAGC | 79 |
| hu_shSIRPA_1707.2 | SIRPα | miR-155 | AUGCUGGCAUACUCCGUGUGG | 80 |
| hu_shSIRPA_1456.2 | SIRPα | miR-155 | AGAUGUUCCGUUCAUUAGAUCC | 81 |
| hu_shSIRPA_1452.2 | SIRPα | miR-155 | UUCCGUUCAUUAGAUCCAGUG | 82 |
| hu_shSIRPA_1444.2 | SIRPα | miR-155 | UAGAUCCAGUGUUCUCAGCGG | 83 |
| hu_shSIRPA_4074.2 | SIRPα | miR-155 | UAAACUCCUGAAACCAUGCUCC | 84 |
| hu_shSIRPA_1457.2 | SIRPα | miR-155 | UAGAUGUUCCGUUCAUUAGAUC | 85 |
| hu_shSIRPA_584.3 | SIRPα | miR-30 | UGAUUGUAGAUUAAUUCCCGGC | 86 |
| hu_shSIRPA_1457.3 | SIRPα | miR-30 | UAGAUGUUCCGUUCAUUAGAUC | 87 |
| hu_shSIRPA_4074.3 | SIRPα | miR-30 | UAAACUCCUGAAACCAUGCUCC | 88 |
| hu_shSTAT6_302.2 | STAT6 | miR-155 | GGAAAGUCGACAUAGAGCCGCU | 89 |
| hu_shSTAT6_499.2 | STAT6 | miR-155 | GAUAUAUGCUCUCAAGGGUGCU | 90 |
| hu_shSTAT6_623.2 | STAT6 | miR-155 | AACUUGAGUUCUUCCUGCUUCC | 91 |
| hu_shSTAT6_626.2 | STAT6 | miR-155 | UUAAACUUGAGUUCUUCCUGCU | 92 |
| hu_shSTAT6_627.2 | STAT6 | miR-155 | CUUAAACUUGAGUUCUUCCUGC | 93 |
| hu_shSTAT6_746.2 | STAT6 | miR-155 | UUAGCAGGAGUUUCUAUCAAGC | 94 |
| hu_shSTAT6_1262.2 | STAT6 | miR-155 | UUGUUGAUGAUUUCUCCAGUGC | 95 |
| hu_shSTAT6_2076.2 | STAT6 | miR-155 | AUUUUUGAGCUGAGCAAGAUCC | 96 |
| hu_shSTAT6_3215.2 | STAT6 | miR-155 | AAAUGUGUCUGUAUGUUCCUGC | 97 |
| hu_shSTAT6_3476.2 | STAT6 | miR-155 | UAUCCUAGGUACAUACACGUUC | 98 |
| hu_shSTAT6_302.4 | STAT6 | Custom | UGGGGAAAGUCGACAUAGAGCCGCU | 99 |
| hu_shSTAT6_499.4 | STAT6 | Custom | UCUGAUAUAUGCUCUCAAGGGUGCU | 100 |
| hu_shSTAT6_623.4 | STAT6 | Custom | UUAAACUUGAGUUCUUCCUGCUUCC | 101 |
| hu_shSTAT6_626.4 | STAT6 | Custom | GUCUUAAACUUGAGUUCUUCCUGCU | 102 |
| hu_shSTAT6_627.4 | STAT6 | Custom | UGUCUUAAACUUGAGUUCUUCCUGC | 103 |
| hu_shSTAT6_746.4 | STAT6 | Custom | CCAUUAGCAGGAGUUUCUAUCAAGC | 104 |
| hu_shSTAT6_1262.4 | STAT6 | Custom | GUGUUGUUGAUGAUUUCUCCAGUGC | 105 |
| hu_shSTAT6_2076.4 | STAT6 | Custom | GAGAUUUUUGAGCUGAGCAAGAUCC | 106 |
| hu_shSTAT6_3215.4 | STAT6 | Custom | UGUAAAUGUGUCUGUAUGUUCCUGC | 107 |
| hu_shSTAT6_3476.4 | STAT6 | Custom | ACAUAUCCUAGGUACAUACACGUUC | 108 |
| hu_shPIK3CG_3862.2 | PIK3CG | miR-155 | GUUUAAACCGAGAAUAUUCUCC | 109 |
| hu_shPIK3CG_2853.2 | PIK3CG | miR-155 | CAAAUAGACUCCAUGAUUCGUA | 110 |
| hu_shPIK3CG_3205.2 | PIK3CG | miR-155 | GUAAUUCCCAAGAAUGUGCCCG | 111 |
| hu_shPIK3CG_2582.2 | PIK3CG | miR-155 | AAAUAACUUGGGAACUGACGUC | 112 |
| hu_shPIK3CG_3644.2 | PIK3CG | miR-155 | UUUAAACCAUAGAACACUAACU | 113 |
| hu_shPIK3CG_2071.2 | PIK3CG | miR-155 | ACUAAAUAGCUUAGGAUAUGCU | 114 |
| hu_shPIK3CG_1548.2 | PIK3CG | miR-155 | AUUUUGAUACUGAACUCAAGCC | 115 |
| hu_shPIK3CG_1677.2 | PIK3CG | miR-155 | UAAUAGAGAAGCUGAACUUUGC | 116 |
| hu_shPIK3CG_5391.2 | PIK3CG | miR-155 | AAUUAUAACCUGGGAAUGGAGC | 117 |
| hu_shPIK3CG_4249.2 | PIK3CG | miR-155 | UUCAUAACCCUGGAACAAGACC | 118 |
| hu_shPIK3CG_3862.4 | PIK3CG | Custom | UCUGUUUAAACCGAGAAUAUUCUCC | 119 |
| hu_shPIK3CG_2853.4 | PIK3CG | Custom | UCCCAAAUAGACUCCAUGAUUCGUA | 120 |
| hu_shPIK3CG_3205.4 | PIK3CG | Custom | UUUGUAAUUCCCAAGAAUGUGCCCG | 121 |
| hu_shPIK3CG_2582.4 | PIK3CG | Custom | GUGAAAUAACUUGGGAACUGACGUC | 122 |
| hu_shPIK3CG_3644.4 | PIK3CG | Custom | UAAUUUAAACCAUAGAACACUAACU | 123 |
| hu_shPIK3CG_2071.4 | PIK3CG | Custom | UGAACUAAAUAGCUUAGGAUAUGCU | 124 |
| hu_shPIK3CG_1548.4 | PIK3CG | Custom | UUGAUUUUGAUACUGAACUCAAGCC | 125 |
| hu_shPIK3CG_1677.4 | PIK3CG | Custom | ACAUAAUAGAGAAGCUGAACUUUGC | 126 |
| hu_shPIK3CG_5391.4 | PIK3CG | Custom | UUAAAUUAUAACCUGGGAAUGGAGC | 127 |
| hu_shPIK3CG_4249.4 | PIK3CG | Custom | AUAUUCAUAACCCUGGAACAAGACC | 128 |
| scramble.b.1 | NA | miR-155 | GUUUCGUUCAUACCUUAGUUGC | 129 |
| scramble.b.3 | NA | Custom | AAAGUUUCGUUCAUACCUUAGUUGC | 130 |
| scramble.c.1 | NA | miR-155 | CUAAGCAUACGCGAUAUUGAGC | 131 |
| scramble.c.3 | NA | Custom | AACCUAAGCAUACGCGAUAUUGAGC | 132 |

TABLE 6b
shRNA Passenger Strand Sequences

| | Target | Scaffold | Passenger Sequence | SEQ ID |
|---|---|---|---|---|
| Name | Gene | Origin | (5′ to 3′) | NO: |
| hu_shSIRPA_583.2 | SIRPα | miR-155 | GGCUGGAGUAAUCUACAAU | 133 |
| hu_shSIRPA_584.2 | SIRPα | miR-155 | GCUGGGGUUAUCUACAAUCA | 134 |
| hu_shSIRPA_1301.2 | SIRPα | miR-155 | GUUGGCCUUGUGAAUGUAU | 135 |
| hu_shSIRPA_1707.2 | SIRPα | miR-155 | CCAUAUGAGAUGCCAGCAU | 136 |
| hu_shSIRPA_1456.2 | SIRPα | miR-155 | GGAUUUAUGAUGGAACAUCU | 137 |
| hu_shSIRPA_1452.2 | SIRPα | miR-155 | CGCUGGUUUAUGAACGGAA | 138 |
| hu_shSIRPA_1444.2 | SIRPα | miR-155 | CCGUUGGAGACUGGAUCUA | 139 |
| hu_shSIRPA_4074.2 | SIRPα | miR-155 | GGGGUAGGUUCAGGAGUUUA | 140 |
| hu_shSIRPA_1457.2 | SIRPα | miR-155 | GAUCUAUGACGGAACAUCUA | 141 |
| hu_shSIRPA_584.3 | SIRPα | miR-30 | GCCGGGAAUUAAUCUACAAUCA | 142 |
| hu_shSIRPA_1457.3 | SIRPα | miR-30 | GAUCUAAUGAACGGAACAUCUA | 143 |
| hu_shSIRPA_4074.3 | SIRPα | miR-30 | GGAGCAUGGUUUCAGGAGUUUA | 144 |
| hu_shSTAT6_302.2 | STAT6 | miR-155 | AGCGGCCUGGUUGACUUUCC | 145 |
| hu_shSTAT6_499.2 | STAT6 | miR-155 | AGCGCUUUGGAGCAUAUAUC | 146 |
| hu_shSTAT6_623.2 | STAT6 | miR-155 | GGAAGUGGAGAAUUCAAGUU | 147 |
| hu_shSTAT6_626.2 | STAT6 | miR-155 | AGCGGGAGAUUCAAGUUUAA | 148 |
| hu_shSTAT6_627.2 | STAT6 | miR-155 | GCAGGAGAGUUAAGUUUAAG | 149 |
| hu_shSTAT6_746.2 | STAT6 | miR-155 | GCUUGGAGAAUUCCUGCUAA | 150 |
| hu_shSTAT6_1262.2 | STAT6 | miR-155 | GCAUUGAGGAUCAUCAACAA | 151 |
| hu_shSTAT6_2076.2 | STAT6 | miR-155 | GGAUUUGCUGGCUCAAAAAU | 152 |
| hu_shSTAT6_3215.2 | STAT6 | miR-155 | GCGGGAUAUCAGACACAUUU | 153 |
| hu_shSTAT6_3476.2 | STAT6 | miR-155 | GAAUGUUGUUACCUAGGAUA | 154 |
| hu_shSTAT6_302.4 | STAT6 | Custom | AGCGGCCUGGUUGACUUUCCCCA | 155 |
| hu_shSTAT6_499.4 | STAT6 | Custom | AGCGCUUUGGAGCAUAUAUCAGA | 156 |
| hu_shSTAT6_623.4 | STAT6 | Custom | GGAAGUGGAGAAUUCAAGUUUAA | 157 |
| hu_shSTAT6_626.4 | STAT6 | Custom | AGCGGGAGAUUCAAGUUUAAGAC | 158 |
| hu_shSTAT6_627.4 | STAT6 | Custom | GCAGGAGAGUUAAGUUUAAGACA | 159 |
| hu_shSTAT6_746.4 | STAT6 | Custom | GCUUGGAGAAUUCCUGCUAAUGG | 160 |
| hu_shSTAT6_1262.4 | STAT6 | Custom | GCAUUGAGGAUCAUCAACAACAC | 161 |
| hu_shSTAT6_2076.4 | STAT6 | Custom | GGAUUUGCUGGCUCAAAAAUCUC | 162 |
| hu_shSTAT6_3215.4 | STAT6 | Custom | GCGGGAUAUCAGACACAUUUACA | 163 |
| hu_shSTAT6_3476.4 | STAT6 | Custom | GAAUGUUGUUACCUAGGAUAUGU | 164 |
| hu_shPIK3CG_3862.2 | PIK3CG | miR-155 | GGAGAGAUUUCGGUUUAAAC | 165 |
| hu_shPIK3CG_2853.2 | PIK3CG | miR-155 | UACGGAUAUGAGUCUAUUUG | 166 |
| hu_shPIK3CG_3205.2 | PIK3CG | miR-155 | CGGGUAGUUUUGGGAAUUAC | 167 |
| hu_shPIK3CG_2582.2 | PIK3CG | miR-155 | GACGUUGUUCGAAGUUAUUU | 168 |
| hu_shPIK3CG_3644.2 | PIK3CG | miR-155 | AGUUGGGUUUGUGGUUUAAA | 169 |
| hu_shPIK3CG_2071.2 | PIK3CG | miR-155 | AGCAUGUCUAGCUAUUUAGU | 170 |
| hu_shPIK3CG_1548.2 | PIK3CG | miR-155 | GGUUUGGUUGGUAUCAAAAU | 171 |
| hu_shPIK3CG_1677.2 | PIK3CG | miR-155 | GCAGAGUCGCUUCUCUAUUA | 172 |
| hu_shPIK3CG_5391.2 | PIK3CG | miR-155 | GCUCCGUUCAGGUUAUAAUU | 173 |
| hu_shPIK3CG_4249.2 | PIK3CG | miR-155 | GGUCUUUUCGGGGUUGUGAA | 174 |
| hu_shPIK3CG_3862.4 | PIK3CG | Custom | GGAGAGAUUUCGGUUUAAACAGA | 175 |
| hu_shPIK3CG_2853.4 | PIK3CG | Custom | UACGGAUAUGAGUCUAUUUGGGA | 176 |
| hu_shPIK3CG_3205.4 | PIK3CG | Custom | CGGGUAGUUUUGGGAAUUACAAA | 177 |
| hu_shPIK3CG_2582.4 | PIK3CG | Custom | GACGUUGUUCGAAGUUAUUUCAC | 178 |
| hu_shPIK3CG_3644.4 | PIK3CG | Custom | AGUUGGGUUUGUGGUUUAAAUUA | 179 |
| hu_shPIK3CG_2071.4 | PIK3CG | Custom | AGCAUGUCUAGCUAUUUAGUUCA | 180 |
| hu_shPIK3CG_1548.4 | PIK3CG | Custom | GGUUUGGUUGGUAUCAAAAUCAA | 181 |
| hu_shPIK3CG_1677.4 | PIK3CG | Custom | GCAGAGUCGCUUCUCUAUUAUGU | 182 |
| hu_shPIK3CG_5391.4 | PIK3CG | Custom | GCUCCGUUCAGGUUAUAAUUUAA | 183 |
| hu_shPIK3CG 4249.4 | PIK3CG | Custom | GGUCUUUUCGGGGUUGUGAAUAU | 184 |
| scramble.b.1 | NA | miR-155 | GCAACUAAGGUAUGAACGAAAC | 185 |
| scramble.b.3 | NA | Custom | GCAACUAAGGUAUGAACGAAACUUU | 186 |
| scramble.c.1 | NA | miR-155 | GCUCAAUAUCGCGUAUGCUUAG | 187 |
| scramble.c.3 | NA | Custom | GCUCAAUAUCGCGUAUGCUUAGGUU | 188 |

TABLE 6c
shRNA Hairpin Sequences

| | Target | Scaffold | Hairpin Sequence | SEQ ID |
|---|---|---|---|---|
| Name | Gene | Origin | (5′ to 3′) | NO: |
| hu_shSIRPA_583.2 | SIRPα | miR-155 | CUGGAGGCUUGCUUUGGGCUGUAUG | 189 |
| | | | CUGAUUGUAGAUUAAUUCCCGGCCU | |
| | | | UUUGGCCUCUGACUGAGGCUGGAGU | |
| | | | AAUCUACAAUCAGGACACAAGGCCC | |
| | | | UUUAUCAGCACUCACA | |
| hu_shSIRPA_584.2 | SIRPα | miR-155 | CUGGAGGCUUGCUUUGGGCUGUAUG | 190 |
| | | | CUGUGAUUGUAGAUUAAUUCCCGGC | |
| | | | UUUUGGCCUCUGACUGAGCUGGGGU | |
| | | | UAUCUACAAUCACAGGACACAAGGC | |
| | | | CCUUUAUCAGCACUCACA | |
| hu_shSIRPA_1301.2 | SIRPα | miR-155 | CUGGAGGCUUGCUUUGGGCUGUAUG | 191 |
| | | | CUGAUACAUUCACCAGGAGCCAGCU | |
| | | | UUUGGCCUCUGACUGAGUUGGCCUU | |
| | | | GUGAAUGUAUCAGGACACAAGGCCC | |
| | | | UUUAUCAGCACUCACA | |
| hu_shSIRPA_1707.2 | SIRPα | miR-155 | CUGGAGGCUUGCUUUGGGCUGUAUG | 192 |
| | | | CUGAUGCUGGCAUACUCCGUGUGGU | |
| | | | UUUGGCCUCUGACUGACCAUAUGAG | |
| | | | AUGCCAGCAUCAGGACACAAGGCCC | |
| | | | UUUAUCAGCACUCACA | |
| hu_shSIRPA_1456.2 | SIRPα | miR-155 | CUGGAGGCUUGCUUUGGGCUGUAUG | 193 |
| | | | CUGAGAUGUUCCGUUCAUUAGAUCC | |
| | | | UUUUGGCCUCUGACUGAGGAUUUAU | |
| | | | GAUGGAACAUCUCAGGACACAAGGC | |
| | | | CCUUUAUCAGCACUCACA | |
| hu_shSIRPA_1452.2 | SIRPα | miR-155 | CUGGAGGCUUGCUUUGGGCUGUAUG | 194 |
| | | | CUGUUCCGUUCAUUAGAUCCAGUGU | |
| | | | UUUGGCCUCUGACUGACGCUGGUUU | |
| | | | AUGAACGGAACAGGACACAAGGCCC | |
| | | | UUUAUCAGCACUCACA | |
| hu_shSIRPA_1444.2 | SIRPα | miR-155 | CUGGAGGCUUGCUUUGGGCUGUAUG | 195 |
| | | | CUGUAGAUCCAGUGUUCUCAGCGGU | |
| | | | UUUGGCCUCUGACUGACCGUUGGAG | |
| | | | ACUGGAUCUACAGGACACAAGGCCC | |
| | | | UUUAUCAGCACUCACA | |
| hu_shSIRPA_4074.2 | SIRPα | miR-155 | CUGGAGGCUUGCUUUGGGCUGUAUG | 196 |
| | | | CUGUAAACUCCUGAAACCAUGCUCC | |
| | | | UUUUGGCCUCUGACUGAGGGGUAGG | |
| | | | UUCAGGAGUUUACAGGACACAAGGC | |
| | | | CCUUUAUCAGCACUCACA | |
| hu_shSIRPA_1457.2 | SIRPα | miR-155 | CUGGAGGCUUGCUUUGGGCUGUAUG | 197 |
| | | | CUGUAGAUGUUCCGUUCAUUAGAUC | |
| | | | UUUUGGCCUCUGACUGAGAUCUAUG | |
| | | | ACGGAACAUCUACAGGACACAAGGC | |
| | | | CCUUUAUCAGCACUCACA | |
| hu_shSIRPA_584.3 | SIRPα | miR-30 | GAGAAGGUAUAUUGCUGUUGACAGU | 198 |
| | | | GAGCGUGAUUGUAGAUUAAUUCCCG | |
| | | | GCUAGUGAAGCCACAGAUGUAGCCG | |
| | | | GGAAUUAAUCUACAAUCAUGCCUAC | |
| | | | UGCCUCGGACUUCAAGGGGCUAGAA | |
| hu_shSIRPA_1457.3 | SIRPα | miR-30 | GAGAAGGUAUAUUGCUGUUGACAGU | 199 |
| | | | GAGCGUAGAUGUUCCGUUCAUUAGA | |
| | | | UCUAGUGAAGCCACAGAUGUAGAUC | |
| | | | UAAUGAACGGAACAUCUAUGCCUAC | |
| | | | UGCCUCGGACUUCAAGGGGCUAGAA | |
| hu_shSIRPA_4074.3 | SIRPα | miR-30 | GAGAAGGUAUAUUGCUGUUGACAGU | 200 |
| | | | GAGCGUAAACUCCUGAAACCAUGCU | |
| | | | CCUAGUGAAGCCACAGAUGUAGGAG | |
| | | | CAUGGUUUCAGGAGUUUAUGCCUAC | |
| | | | UGCCUCGGACUUCAAGGGGCUAGAA | |
| hu_shSTAT6_302.2 | STAT6 | miR-155 | CUGGAGGCUUGCUUUGGGCUGUAUG | 201 |
| | | | CUGGGAAAGUCGACAUAGAGCCGCU | |
| | | | UUUUGGCCUCUGACUGAAGCGGCCU | |
| | | | GGUUGACUUUCCCAGGACACAAGGC | |
| | | | CCUUUAUCAGCACUCACA | |
| hu_shSTAT6_499.2 | STAT6 | miR-155 | CUGGAGGCUUGCUUUGGGCUGUAUG | 202 |
| | | | CUGGAUAUAUGCUCUCAAGGGUGCU | |
| | | | UUUUGGCCUCUGACUGAAGCGCUUU | |
| | | | GGAGCAUAUAUCCAGGACACAAGGC | |
| | | | CCUUUAUCAGCACUCACA | |
| hu_shSTAT6_623.2 | STAT6 | miR-155 | CUGGAGGCUUGCUUUGGGCUGUAUG | 203 |
| | | | CUGAACUUGAGUUCUUCCUGCUUCC | |
| | | | UUUUGGCCUCUGACUGAGGAAGUGG | |
| | | | AGAAUUCAAGUUCAGGACACAAGGC | |
| | | | CCUUUAUCAGCACUCACA | |
| hu shSTAT6_626.2 | STAT6 | miR-155 | CUGGAGGCUUGCUUUGGGCUGUAUG | 204 |
| | | | CUGUUAAACUUGAGUUCUUCCUGCU | |
| | | | UUUUGGCCUCUGACUGAAGCGGGAG | |
| | | | AUUCAAGUUUAACAGGACACAAGGC | |
| | | | CCUUUAUCAGCACUCACA | |
| hu_shSTAT6_627.2 | STAT6 | miR-155 | CUGGAGGCUUGCUUUGGGCUGUAUG | 205 |
| | | | CUGCUUAAACUUGAGUUCUUCCUGC | |
| | | | UUUUGGCCUCUGACUGAGCAGGAGA | |
| | | | GUUAAGUUUAAGCAGGACACAAGGC | |
| | | | CCUUUAUCAGCACUCACA | |
| hu_shSTAT6_746.2 | STAT6 | miR-155 | CUGGAGGCUUGCUUUGGGCUGUAUG | 206 |
| | | | CUGUUAGCAGGAGUUUCUAUCAAGC | |
| | | | UUUUGGCCUCUGACUGAGCUUGGAG | |
| | | | AAUUCCUGCUAACAGGACACAAGGC | |
| | | | CCUUUAUCAGCACUCACA | |
| hu_shSTAT6_1262.2 | STAT6 | miR-155 | CUGGAGGCUUGCUUUGGGCUGUAUG | 207 |
| | | | CUGUUGUUGAUGAUUUCUCCAGUGC | |
| | | | UUUUGGCCUCUGACUGAGCAUUGAG | |
| | | | GAUCAUCAACAACAGGACACAAGGC | |
| | | | CCUUUAUCAGCACUCACA | |
| hu_shSTAT6_2076.2 | STAT6 | miR-155 | CUGGAGGCUUGCUUUGGGCUGUAUG | 208 |
| | | | CUGAUUUUUGAGCUGAGCAAGAUCC | |
| | | | UUUUGGCCUCUGACUGAGGAUUUGC | |
| | | | UGGCUCAAAAAUCAGGACACAAGGC | |
| | | | CCUUUAUCAGCACUCACA | |
| hu_shSTAT6_3215.2 | STAT6 | miR-155 | CUGGAGGCUUGCUUUGGGCUGUAUG | 209 |
| | | | CUGAAAUGUGUCUGUAUGUUCCUGC | |
| | | | UUUUGGCCUCUGACUGAGCGGGAUA | |
| | | | UCAGACACAUUUCAGGACACAAGGC | |
| | | | CCUUUAUCAGCACUCACA | |
| hu_shSTAT6_3476.2 | STAT6 | miR-155 | CUGGAGGCUUGCUUUGGGCUGUAUG | 210 |
| | | | CUGUAUCCUAGGUACAUACACGUUC | |
| | | | UUUUGGCCUCUGACUGAGAAUGUUG | |
| | | | UUACCUAGGAUACAGGACACAAGGC | |
| | | | CCUUUAUCAGCACUCACA | |
| hu_shSTAT6_302.4 | STAT6 | Custom | CUGGAGGCUUGCUUUGCGAAUCUCU | 211 |
| | | | GGGGAAAGUCGACAUAGAGCCGCUU | |
| | | | GUGUUAUUCUUGAAGCGGCCUGGUU | |
| | | | GACUUUCCCCAGCGAUUCGCUUUAU | |
| | | | CGUCACUCACA | |
| hu_shSTAT6_499.4 | STAT6 | Custom | CUGGAGGCUUGCUUUGCGAAUCUCU | 212 |
| | | | CUGAUAUAUGCUCUCAAGGGUGCUU | |
| | | | GUGUUAUUCUUGAAGCGCUUUGGAG | |
| | | | CAUAUAUCAGAGCGAUUCGCUUUAU | |
| | | | CGUCACUCACA | |
| hu_shSTAT6_623.4 | STAT6 | Custom | CUGGAGGCUUGCUUUGCGAAUCUCU | 213 |
| | | | UAAACUUGAGUUCUUCCUGCUUCCU | |
| | | | GUGUUAUUCUUGAGGAAGUGGAGAA | |
| | | | UUCAAGUUUAAGCGAUUCGCUUUAU | |
| | | | CGUCACUCACA | |
| hu_shSTAT6_626.4 | STAT6 | Custom | CUGGAGGCUUGCUUUGCGAAUCUCG | 214 |
| | | | UCUUAAACUUGAGUUCUUCCUGCUU | |
| | | | GUGUUAUUCUUGAAGCGGGAGAUUC | |
| | | | AAGUUUAAGACGCGAUUCGCUUUAU | |
| | | | CGUCACUCACA | |
| hu_shSTAT6_627.4 | STAT6 | Custom | CUGGAGGCUUGCUUUGCGAAUCUCU | 215 |
| | | | GUCUUAAACUUGAGUUCUUCCUGCU | |
| | | | GUGUUAUUCUUGAGCAGGAGAGUUA | |
| | | | AGUUUAAGACAGCGAUUCGCUUUAU | |
| | | | CGUCACUCACA | |
| hu_shSTAT6_746.4 | STAT6 | Custom | CUGGAGGCUUGCUUUGCGAAUCUCC | 216 |
| | | | CAUUAGCAGGAGUUUCUAUCAAGCU | |
| | | | GUGUUAUUCUUGAGCUUGGAGAAUU | |
| | | | CCUGCUAAUGGGCGAUUCGCUUUAU | |
| | | | CGUCACUCACA | |
| hu_shSTAT6_1262.4 | STAT6 | Custom | CUGGAGGCUUGCUUUGCGAAUCUCG | 217 |
| | | | UGUUGUUGAUGAUUUCUCCAGUGCU | |
| | | | GUGUUAUUCUUGAGCAUUGAGGAUC | |
| | | | AUCAACAACACGCGAUUCGCUUUAU | |
| | | | CGUCACUCACA | |
| hu_shSTAT6_2076.4 | STAT6 | Custom | CUGGAGGCUUGCUUUGCGAAUCUCG | 218 |
| | | | AGAUUUUUGAGCUGAGCAAGAUCCU | |
| | | | GUGUUAUUCUUGAGGAUUUGCUGGC | |
| | | | UCAAAAAUCUCGCGAUUCGCUUUAU | |
| | | | CGUCACUCACA | |
| hu_shSTAT6_3215.4 | STAT6 | Custom | CUGGAGGCUUGCUUUGCGAAUCUCU | 219 |
| | | | GUAAAUGUGUCUGUAUGUUCCUGCU | |
| | | | GUGUUAUUCUUGAGCGGGAUAUCAG | |
| | | | ACACAUUUACAGCGAUUCGCUUUAU | |
| | | | CGUCACUCACA | |
| hu_shSTAT6_3476.4 | STAT6 | Custom | CUGGAGGCUUGCUUUGCGAAUCUCA | 220 |
| | | | CAUAUCCUAGGUACAUACACGUUCU | |
| | | | GUGUUAUUCUUGAGAAUGUUGUUAC | |
| | | | CUAGGAUAUGUGCGAUUCGCUUUAU | |
| | | | CGUCACUCACA | |
| hu_shPIK3CG_3862.2 | PIK3CG | miR-155 | CUGGAGGCUUGCUUUGGGCUGUAUG | 221 |
| | | | CUGGUUUAAACCGAGAAUAUUCUCC | |
| | | | UUUUGGCCUCUGACUGAGGAGAGAU | |
| | | | UUCGGUUUAAACCAGGACACAAGGC | |
| | | | CCUUUAUCAGCACUCACA | |
| hu_shPIK3CG_2853.2 | PIK3CG | miR-155 | CUGGAGGCUUGCUUUGGGCUGUAUG | 222 |
| | | | CUGCAAAUAGACUCCAUGAUUCGUA | |
| | | | UUUUGGCCUCUGACUGAUACGGAUA | |
| | | | UGAGUCUAUUUGCAGGACACAAGGC | |
| | | | CCUUUAUCAGCACUCACA | |
| hu_shPIK3CG_3205.2 | PIK3CG | miR-155 | CUGGAGGCUUGCUUUGGGCUGUAUG | 223 |
| | | | CUGGUAAUUCCCAAGAAUGUGCCCG | |
| | | | UUUUGGCCUCUGACUGACGGGUAGU | |
| | | | UUUGGGAAUUACCAGGACACAAGGC | |
| | | | CCUUUAUCAGCACUCACA | |
| hu_shPIK3CG_2582.2 | PIK3CG | miR-155 | CUGGAGGCUUGCUUUGGGCUGUAUG | 224 |
| | | | CUGAAAUAACUUGGGAACUGACGUC | |
| | | | UUUUGGCCUCUGACUGAGACGUUGU | |
| | | | UCGAAGUUAUUUCAGGACACAAGGC | |
| | | | CCUUUAUCAGCACUCACA | |
| hu_shPIK3CG_3644.2 | PIK3CG | miR-155 | CUGGAGGCUUGCUUUGGGCUGUAUG | 225 |
| | | | CUGUUUAAACCAUAGAACACUAACU | |
| | | | UUUUGGCCUCUGACUGAAGUUGGGU | |
| | | | UUGUGGUUUAAACAGGACACAAGGC | |
| | | | CCUUUAUCAGCACUCACA | |
| hu_shPIK3CG_2071.2 | PIK3CG | miR-155 | CUGGAGGCUUGCUUUGGGCUGUAUG | 226 |
| | | | CUGACUAAAUAGCUUAGGAUAUGCU | |
| | | | UUUUGGCCUCUGACUGAAGCAUGUC | |
| | | | UAGCUAUUUAGUCAGGACACAAGGC | |
| | | | CCUUUAUCAGCACUCACA | |
| hu_shPIK3CG_1548.2 | PIK3CG | miR-155 | CUGGAGGCUUGCUUUGGGCUGUAUG | 227 |
| | | | CUGAUUUUGAUACUGAACUCAAGCC | |
| | | | UUUUGGCCUCUGACUGAGGUUUGGU | |
| | | | UGGUAUCAAAAUCAGGACACAAGGC | |
| | | | CCUUUAUCAGCACUCACA | |
| hu_shPIK3CG_1677.2 | PIK3CG | miR-155 | CUGGAGGCUUGCUUUGGGCUGUAUG | 228 |
| | | | CUGUAAUAGAGAAGCUGAACUUUGC | |
| | | | UUUUGGCCUCUGACUGAGCAGAGUC | |
| | | | GCUUCUCUAUUACAGGACACAAGGC | |
| | | | CCUUUAUCAGCACUCACA | |
| hu_shPIK3CG_5391.2 | PIK3CG | miR-155 | CUGGAGGCUUGCUUUGGGCUGUAUG | 229 |
| | | | CUGAAUUAUAACCUGGGAAUGGAGC | |
| | | | UUUUGGCCUCUGACUGAGCUCCGUU | |
| | | | CAGGUUAUAAUUCAGGACACAAGGC | |
| | | | CCUUUAUCAGCACUCACA | |
| hu_shPIK3CG_4249.2 | PIK3CG | miR-155 | CUGGAGGCUUGCUUUGGGCUGUAUG | 230 |
| | | | CUGUUCAUAACCCUGGAACAAGACC | |
| | | | UUUUGGCCUCUGACUGAGGUCUUUU | |
| | | | CGGGGUUGUGAACAGGACACAAGGC | |
| | | | CCUUUAUCAGCACUCACA | |
| hu_shPIK3CG_3862.4 | PIK3CG | Custom | CUGGAGGCUUGCUUUGCGAAUCUCU | 231 |
| | | | CUGUUUAAACCGAGAAUAUUCUCCU | |
| | | | GUGUUAUUCUUGAGGAGAGAUUUCG | |
| | | | GUUUAAACAGAGCGAUUCGCUUUAU | |
| | | | CGUCACUCACA | |
| hu_shPIK3CG_2853.4 | PIK3CG | Custom | CUGGAGGCUUGCUUUGCGAAUCUCU | 232 |
| | | | CCCAAAUAGACUCCAUGAUUCGUAU | |
| | | | GUGUUAUUCUUGAUACGGAUAUGAG | |
| | | | UCUAUUUGGGAGCGAUUCGCUUUAU | |
| | | | CGUCACUCACA | |
| hu_shPIK3CG_3205.4 | PIK3CG | Custom | CUGGAGGCUUGCUUUGCGAAUCUCU | 233 |
| | | | UUGUAAUUCCCAAGAAUGUGCCCGU | |
| | | | GUGUUAUUCUUGACGGGUAGUUUUG | |
| | | | GGAAUUACAAAGCGAUUCGCUUUAU | |
| | | | CGUCACUCACA | |
| hu_shPIK3CG_2582.4 | PIK3CG | Custom | CUGGAGGCUUGCUUUGCGAAUCUCG | 234 |
| | | | UGAAAUAACUUGGGAACUGACGUCU | |
| | | | GUGUUAUUCUUGAGACGUUGUUCGA | |
| | | | AGUUAUUUCACGCGAUUCGCUUUAU | |
| | | | CGUCACUCACA | |
| hu_shPIK3CG_3644.4 | PIK3CG | Custom | CUGGAGGCUUGCUUUGCGAAUCUCU | 235 |
| | | | AAUUUAAACCAUAGAACACUAACUU | |
| | | | GUGUUAUUCUUGAAGUUGGGUUUGU | |
| | | | GGUUUAAAUUAGCGAUUCGCUUUAU | |
| | | | CGUCACUCACA | |
| hu_shPIK3CG_2071.4 | PIK3CG | Custom | CUGGAGGCUUGCUUUGCGAAUCUCU | 236 |
| | | | GAACUAAAUAGCUUAGGAUAUGCUU | |
| | | | GUGUUAUUCUUGAAGCAUGUCUAGC | |
| | | | UAUUUAGUUCAGCGAUUCGCUUUAU | |
| | | | CGUCACUCACA | |
| hu_shPIK3CG_1548.4 | PIK3CG | Custom | CUGGAGGCUUGCUUUGCGAAUCUCU | 237 |
| | | | UGAUUUUGAUACUGAACUCAAGCCU | |
| | | | GUGUUAUUCUUGAGGUUUGGUUGGU | |
| | | | AUCAAAAUCAAGCGAUUCGCUUUAU | |
| | | | CGUCACUCACA | |
| hu_shPIK3CG_1677.4 | PIK3CG | Custom | CUGGAGGCUUGCUUUGCGAAUCUCA | 238 |
| | | | CAUAAUAGAGAAGCUGAACUUUGCU | |
| | | | GUGUUAUUCUUGAGCAGAGUCGCUU | |
| | | | CUCUAUUAUGUGCGAUUCGCUUUAU | |
| | | | CGUCACUCACA | |
| hu_shPIK3CG_5391.4 | PIK3CG | Custom | CUGGAGGCUUGCUUUGCGAAUCUCU | 239 |
| | | | UAAAUUAUAACCUGGGAAUGGAGCU | |
| | | | GUGUUAUUCUUGAGCUCCGUUCAGG | |
| | | | UUAUAAUUUAAGCGAUUCGCUUUAU | |
| | | | CGUCACUCACA | |
| hu_shPIK3CG_4249.4 | PIK3CG | Custom | CUGGAGGCUUGCUUUGCGAAUCUCA | 240 |
| | | | UAUUCAUAACCCUGGAACAAGACCU | |
| | | | GUGUUAUUCUUGAGGUCUUUUCGGG | |
| | | | GUUGUGAAUAUGCGAUUCGCUUUAU | |
| | | | CGUCACUCACA | |
| scramble.b.1 | NA | miR-155 | CUGGAGGCUUGCUUUGGGCUGUAUG | 241 |
| | | | CUGGUUUCGUUCAUACCUUAGUUGC | |
| | | | UUUUGGCCUCUGACUGAGCAACUAA | |
| | | | GGUAUGAACGAAACCAGGACACAAG | |
| | | | GCCCUUUAUCAGCACUCACA | |
| scramble.b.3 | NA | Custom | CUGGAGGCUUGCUUUGCGAAUCUCA | 242 |
| | | | AAGUUUCGUUCAUACCUUAGUUGCU | |
| | | | GUGUUAUUCUUGAGCAACUAAGGUA | |
| | | | UGAACGAAACUUUGCGAUUCGCUUU | |
| | | | AUCGUCACUCACA | |
| scramble.c.1 | NA | miR-155 | CUGGAGGCUUGCUUUGGGCUGUAUG | 243 |
| | | | CUGCUAAGCAUACGCGAUAUUGAGC | |
| | | | UUUUGGCCUCUGACUGAGCUCAAUA | |
| | | | UCGCGUAUGCUUAGCAGGACACAAG | |
| | | | GCCCUUUAUCAGCACUCACA | |
| scramble.c.3 | NA | Custom | CUGGAGGCUUGCUUUGCGAAUCUCA | 244 |
| | | | ACCUAAGCAUACGCGAUAUUGAGCU | |
| | | | GUGUUAUUCUUGAGCUCAAUAUCGC | |
| | | | GUAUGCUUAGGUUGCGAUUCGCUUU | |
| | | | AUCGUCACUCACA | |

TABLE 6d
shRNA Scaffold Sequences

| Scaffold | 5′ Scaffold Sequence | Loop Sequence | 3′ Scaffold Sequence |
|---|---|---|---|
| Origin | (5′ to 3′) | (5′ to 3′) | (5′ to 3′) |
| miR-155 | CUGGAGGCUUGCUUU | UUUUGGCCUCUGAC | CAGGACACAAGGCCCUUUA |
| | GGGCUGUAUGCUG | UGA (SEQ ID NO: 248) | UCAGCACUCACA (SEQ ID |
| | (SEQ ID NO: 245) | | NO: 251) |
| miR-30 | GAGAAGGUAUAUUGC | UAGUGAAGCCACAG | UGCCUACUGCCUCGGACUU |
| | UGUUGACAGUGAGCG | AUGUA (SEQ ID NO: | CAAGGGGCUAGAA (SEQ ID |
| | (SEQ ID NO: 246) | 249) | NO: 252) |
| Custom | CUGGAGGCUUGCUUU | UGUGUUAUUCUUGA | GCGAUUCGCUUUAUCGUCA |
| | GCGAAUCUC (SEQ ID | (SEQ ID NO: 250) | CUCACA (SEQ ID NO: 253) |
| | NO: 247) | | |

TABLE 6e
Tandem shRNA Sequences

| | | Tandem Sequence |
|---|---|---|
| Name | hu_SIRPa_multi_1 | GAGAAGGUAUAUUGCUGUUGACAGUG |
| | | AGCGAUAGAUGUUCCGUUCAUUAGAUC |
| Target Gene(s) | SIRPα | UAGUGAAGCCACAGAUGUAGAUCUAAU |
| | | GAACGGAACAUCUAGUGCCUACUGCCU |
| | | CGGACUUCAAGGGGCUAAUGUCUAGAA |
| Induced Phenotype | N/A | AACGAAGGUAUAUUGCUGUUGACAGU |
| | | GAGCGAUGAUUGUAGAUUAAUUCCCGG |
| | | CUAGUGAAGCCACAGAUGUAGCCGGGA |
| shRNA1 | hu_shSIRPA_1457.3 | AUUAAUCUACAAUCAGUGCCUACUGCC |
| | | UCGGACUUCAAGGGGCUAUUGCGGCCG |
| | | CAACGAAGGUAUAUUGCUGUUGACAGU |
| shRNA2 | hu_shSIRPA_584.3 | GAGCGAUAAACUCCUGAAACCAUGCUC |
| | | CUAGUGAAGCCACAGAUGUAGGAGCAU |
| shRNA3 | hu_shSIRPA_4074.3 | GGUUUCAGGAGUUUAGUGCCUACUGCC |
| | | UCGGACUUCAAGGGGCUAGAA (SEQ ID |
| | | NO: 254) |
| Name | hu_SIRPa_multi_2 | GAGAAGGUAUAUUGCUGUUGACAGUG |
| | | AGCGAUAGAUGUUCCGUUCAUUAGAUC |
| | | UAGUGAAGCCACAGAUGUAGAUCUAAU |
| Target Gene(s) | SIRPα | GAACGGAACAUCUAGUGCCUACUGCCU |
| | | CGGACUUCAAGGGGCUAAUGUCUAGAA |
| Induced Phenotype | N/A | AACCUGGAGGCUUGCUUUGGGCUGUAU |
| | | GCUGUGAUUGUAGAUUAAUUCCCGGCU |
| | | UUUGGCCUCUGACUGAGCUGGGGUUAU |
| shRNA1 | hu_shSIRPA_1457.3 | CUACAAUCACAGGACACAAGGCCCUUU |
| | | AUCAGCACUCACAUUGCGGCCGCAACG |
| shRNA2 | hu_shSIRPA_584.2 | AAGGUAUAUUGCUGUUGACAGUGAGC |
| | | GAUAAACUCCUGAAACCAUGCUCCUAG |
| | | UGAAGCCACAGAUGUAGGAGCAUGGUU |
| | | UCAGGAGUUUAGUGCCUACUGCCUCGG |
| shRNA3 | hu_shSIRPA_4074.3 | ACUUCAAGGGGCUAGAA (SEQ ID NO: |
| | | 255) |
| Name | hu_SIRPa_multi_3 | CUGGAGGCUUGCUUUGGGCUGUAUGCU |
| | | GUAGAUGUUCCGUUCAUUAGAUCUUUU |
| | | GGCCUCUGACUGAGAUCUAUGACGGAA |
| Target Gene(s) | SIRPα | CAUCUACAGGACACAAGGCCCUUUAUC |
| | | AGCACUCACAAUGUCUAGAAAACGAAG |
| Induced Phenotype | N/A | GUAUAUUGCUGUUGACAGUGAGCGAU |
| | | GAUUGUAGAUUAAUUCCCGGCUAGUGA |
| | | AGCCACAGAUGUAGCCGGGAAUUAAUC |
| shRNA1 | hu_shSIRPA_1457.2 | UACAAUCAGUGCCUACUGCCUCGGACU |
| | | UCAAGGGGCUAUUGCGGCCGCAACCUG |
| shRNA2 | hu_shSIRPA_584.3 | GAGGCUUGCUUUGGGCUGUAUGCUGUA |
| | | AACUCCUGAAACCAUGCUCCUUUUGGC |
| | | CUCUGACUGAGGGGUAGGUUCAGGAGU |
| shRNA3 | hu_shSIRPA_4074.2 | UUACAGGACACAAGGCCCUUUAUCAGC |
| | | ACUCACA (SEQ ID NO: 256) |
| Name | hu_SIRPa_multi_4 | CUGGAGGCUUGCUUUGGGCUGUAUGCU |
| | | GUAGAUGUUCCGUUCAUUAGAUCUUUU |
| | | GGCCUCUGACUGAGAUCUAUGACGGAA |
| Target Gene(s) | SIRPα | CAUCUACAGGACACAAGGCCCUUUAUC |
| | | AGCACUCACAAUGUCUAGAAAACCUGG |
| Induced Phenotype | N/A | AGGCUUGCUUUGGGCUGUAUGCUGUGA |
| | | UUGUAGAUUAAUUCCCGGCUUUUGGCC |
| | | UCUGACUGAGCUGGGGUUAUCUACAAU |
| shRNA1 | hu_shSIRPA_1457.2 | CACAGGACACAAGGCCCUUUAUCAGCA |
| | | CUCACAUUGCGGCCGCAACCUGGAGGC |
| shRNA2 | hu_shSIRPA_584.2 | UUGCUUUGGGCUGUAUGCUGUAAACUC |
| | | CUGAAACCAUGCUCCUUUUGGCCUCUG |
| | | ACUGAGGGGUAGGUUCAGGAGUUUAC |
| shRNA3 | hu_shSIRPA_4074.2 | AGGACACAAGGCCCUUUAUCAGCACUC |
| | | ACA (SEQ ID NO: 257) |
| Name | hu_SIRPa_125a_1 | CUGGAGGCUUGCUUUGGGCUGUAUGCU |
| | | GUAGAUGUUCCGUUCAUUAGAUCUUUU |
| | | GGCCUCUGACUGAGATCTATGACGGAA |
| Target Gene(s) | SIRPα | CATCTACAGGACACAAGGCCCUUUAUC |
| | | AGCACUCACAATGTCTAGAAAACCUGG |
| Induced Phenotype | N/A | AGGCUUGCUUUGGGCUGUAUGCUGUGA |
| | | UUGUAGAUUAAUUCCCGGCUUUUGGCC |
| | | UCUGACUGAGCUGGGGTTATCTACAAT |
| shRNA1 | hu_shSIRPa_1457.2 | CACAGGACACAAGGCCCUUUAUCAGCA |
| | | CUCACATTGCGGCCGCAACTCTACCGGG |
| shRNA2 | hu_shSIRPa_584.2 | CCACCGCACACCATGTTGCCAGTCTCTA |
| | | GGTCCCTGAGACCCTTTAACCTGTGAGG |
| | | ACATCCAGGGTCACAGGTGAGGTTCTTG |
| shRNA3 | hsa-miR-125a | GGAGCCTGGCGTCTGGCCCAACCACACA |
| | | CCTGGGGAATTGCTG (SEQ ID NO: 258) |
| Name | hu_SIRPa_130_1 | CUGGAGGCUUGCUUUGGGCUGUAUGCU |
| | | GUAGAUGUUCCGUUCAUUAGAUCUUUU |
| | | GGCCUCUGACUGAGATCTATGACGGAA |
| Target Gene(s) | SIRPα | CATCTACAGGACACAAGGCCCUUUAUC |
| | | AGCACUCACAATGTCTAGAAAACCUGG |
| Induced Phenotype | M1 | AGGCUUGCUUUGGGCUGUAUGCUGUGA |
| | | UUGUAGAUUAAUUCCCGGCUUUUGGCC |
| | | UCUGACUGAGCUGGGGTTATCTACAAT |
| shRNA1 | hu_shSIRPa_1457.2 | CACAGGACACAAGGCCCUUUAUCAGCA |
| | | CUCACATTGCGGCCGCAACTGAGGAGG |
| | | CAGGGCCGGCATGCCTCTGCTGCTGGCC |
| shRNA2 | hu_shSIRPa_584.2 | AGAGCTCTTTTCACATTGTGCTACTGTCT |
| | | GCACCTGTCACTAGCAGTGCAATGTTAA |
| shRNA3 | hsa-miR-130a | AAGGGCATTGGCCGTGTAGTGCTACCCA |
| | | GCGCTGGCTGCCTCCTCA (SEQ ID NO: |
| | | 259) |
| Name | hu_SIRPa_155_a | CUGGAGGCUUGCUUUGGGCUGUAUGCU |
| | | GUAGAUGUUCCGUUCAUUAGAUCUUUU |
| Target Gene(s) | SIRPα | GGCCUCUGACUGAGATCTATGACGGAA |
| | | CATCTACAGGACACAAGGCCCUUUAUC |
| | | AGCACUCACAATGTCTAGAAAACCUGG |
| Induced Phenotype | M1 | AGGCUUGCUUUGGGCUGUAUGCUGUGA |
| | | UUGUAGAUUAAUUCCCGGCUUUUGGCC |
| | | UCUGACUGAGCUGGGGTTATCTACAAT |
| shRNA1 | hu_shSIRPa_1457.2 | CACAGGACACAAGGCCCUUUAUCAGCA |
| | | CUCACATTGCGGCCGCAACCTGAAGGCT |
| shRNA2 | hu_shSIRPa_584.2 | TGCTGTAGGCTGTATGCTGTTAATGCTA |
| | | ATCGTGATAGGGGTTTTTGCCTCCAACT |
| shRNA3 | hsa-miR-155 | GACTCCTACATATTAGCATTAACAGTGT |
| | | ATGATGCCTGTTACTAGCATTC (SEQ ID |
| | | NO: 260) |
| Name | hu_24_1 | CATCCTGCTGCCTGGCCTCCCTGGGCTC |
| | | TGCCTCCCGTGCCTACTGAGCTGAAACA |
| Target Gene(s) | N/A | CAGTTGGTTTGTGTACACTGGCTCAGTT |
| | | CAGCAGGAACAGGGGTCAAGCCCCCTT |
| | | GGAGCCTGCAGCATGTCTAGAAAACCU |
| Induced Phenotype | M2 | GGAGGCUUGCUUUGGGCUGUAUGCUGG |
| | | UUUCGUUCAUACCUUAGUUGCUUUUGG |
| | | CCUCUGACUGAGCAACUAAGGUAUGAA |
| shRNA1 | hsa-miR-24-2 | CGAAACCAGGACACAAGGCCCUUUAUC |
| | | AGCACUCACATTGCGGCCGCAACCUGG |
| shRNA2 | scramble.b.1 | AGGCUUGCUUUGGGCUGUAUGCUGCUA |
| | | AGCAUACGCGAUAUUGAGCUUUUGGCC |
| | | UCUGACUGAGCUCAAUAUCGCGUAUGC |
| shRNA3 | scramble.c.1 | UUAGCAGGACACAAGGCCCUUUAUCAG |
| | | CACUCACA (SEQ ID NO: 261) |
| Name | hu_145_1 | TTGGCGCTGAAGGCCACTCGCTCCCACC |
| | | TTGTCCTCACGGTCCAGTTTTCCCAGGA |
| | | ATCCCTTAGATGCTAAGATGGGGATTCC |
| Target Gene(s) | N/A | TGGAAATACTGTTCTTGAGGTCATGGTT |
| | | TCACAGCTGGATTTGCCTCCTTCCCATGT |
| Induced Phenotype | M2 | CTAGAAAACCUGGAGGCUUGCUUUGGG |
| | | CUGUAUGCUGGUUUCGUUCAUACCUUA |
| | | GUUGCUUUUGGCCUCUGACUGAGCAAC |
| shRNA1 | hsa-miR-145 | UAAGGUAUGAACGAAACCAGGACACAA |
| | | GGCCCUUUAUCAGCACUCACATTGCGG |
| | | CCGCAACCUGGAGGCUUGCUUUGGGCU |
| shRNA2 | scramble.b.1 | GUAUGCUGCUAAGCAUACGCGAUAUUG |
| | | AGCUUUUGGCCUCUGACUGAGCUCAAU |
| shRNA3 | scramble.c.1 | AUCGCGUAUGCUUAGCAGGACACAAGG |
| | | CCCUUUAUCAGCACUCACA (SEQ ID |
| | | NO: 262) |
| Name | hu_146b_1 | GTCCAGGCTGAAAGAACTTTGGCCACCT |
| | | GGCACTGAGAACTGAATTCCATAGGCTG |
| | | TGAGCTCTAGCAATGCCCTGTGGACTCA |
| Target Gene(s) | N/A | GTTCTGGTGCCCGGCAGTGCTACAACAT |
| | | CAATGCCAAGGATGTCTAGAAAACCUG |
| Induced Phenotype | M2 | GAGGCUUGCUUUGGGCUGUAUGCUGGU |
| | | UUCGUUCAUACCUUAGUUGCUUUUGGC |
| shRNA1 | hsa-miR-146b | CUCUGACUGAGCAACUAAGGUAUGAAC |
| | | GAAACCAGGACACAAGGCCCUUUAUCA |
| | | GCACUCACATTGCGGCCGCAACCUGGA |
| shRNA2 | scramble.b.1 | GGCUUGCUUUGGGCUGUAUGCUGCUAA |
| | | GCAUACGCGAUAUUGAGCUUUUGGCCU |
| shRNA3 | scramble.c.1 | CUGACUGAGCUCAAUAUCGCGUAUGCU |
| | | UAGCAGGACACAAGGCCCUUUAUCAGC |
| | | ACUCACA (SEQ ID NO: 263) |
| Name | hu_93_1 | TGTCCTCCCGTCTTGGACCTCAGTCCTG |
| | | GGGGCTCCAAAGTGCTGTTCGTGCAGGT |
| Target Gene(s) | N/A | AGTGTGATTACCCAACCTACTGCTGAGC |
| | | TAGCACTTCCCGAGCCCCCGGGACACGT |
| | | TCTCTCTGCCAATTGTCTATGTCTAGAA |
| Induced Phenotype | M2 | AACCUGGAGGCUUGCUUUGGGCUGUAU |
| | | GCUGGUUUCGUUCAUACCUUAGUUGCU |
| | | UUUGGCCUCUGACUGAGCAACUAAGGU |
| shRNA1 | hsa-miR-93 | AUGAACGAAACCAGGACACAAGGCCCU |
| | | UUAUCAGCACUCACATTGCGGCCGCAA |
| shRNA2 | scramble.b.1 | CCUGGAGGCUUGCUUUGGGCUGUAUGC |
| | | UGCUAAGCAUACGCGAUAUUGAGCUUU |
| | | UGGCCUCUGACUGAGCUCAAUAUCGCG |
| shRNA3 | scramble.c.1 | UAUGCUUAGCAGGACACAAGGCCCUUU |
| | | AUCAGCACUCACA (SEQ ID NO: 264) |
| Name | hu_223_1 | TTCCCCACAGAAGCTCTTGGCCTGGCCT |
| | | CCTGCAGTGCCACGCTCCGTGTATTTGA |
| | | CAAGCTGAGTTGGACACTCCATGTGGTA |
| Target Gene(s) | N/A | GAGTGTCAGTTTGTCAAATACCCCAAGT |
| | | GCGGCACATGCTTACCAGCTCTAGGCCA |
| Induced Phenotype | M2 | GGGCAGATGGATGTCTAGAAAACCUGG |
| | | AGGCUUGCUUUGGGCUGUAUGCUGGUU |
| | | UCGUUCAUACCUUAGUUGCUUUUGGCC |
| shRNA1 | hsa-miR-223 | UCUGACUGAGCAACUAAGGUAUGAACG |
| | | AAACCAGGACACAAGGCCCUUUAUCAG |
| | | CACUCACATTGCGGCCGCAACCUGGAG |
| shRNA2 | scramble.b.1 | GCUUGCUUUGGGCUGUAUGCUGCUAAG |
| | | CAUACGCGAUAUUGAGCUUUUGGCCUC |
| shRNA3 | scramble.c.1 | UGACUGAGCUCAAUAUCGCGUAUGCUU |
| | | AGCAGGACACAAGGCCCUUUAUCAGCA |
| | | CUCACA (SEQ ID NO: 265) |
| Name | hu_M2_tandem_1 | CATCCTGCTGCCTGGCCTCCCTGGGCTC |
| | | TGCCTCCCGTGCCTACTGAGCTGAAACA |
| Target Gene(s) | N/A | CAGTTGGTTTGTGTACACTGGCTCAGTT |
| | | CAGCAGGAACAGGGGTCAAGCCCCCTT |
| Induced Phenotype | M2 | GGAGCCTGCAGCATGTCTAGAAAACTGT |
| | | CCTCCCGTCTTGGACCTCAGTCCTGGGG |
| shRNA1 | hsa-miR-24-2 | GCTCCAAAGTGCTGTTCGTGCAGGTAGT |
| | | GTGATTACCCAACCTACTGCTGAGCTAG |
| | | CACTTCCCGAGCCCCCGGGACACGTTCT |
| shRNA2 | hsa-miR-93 | CTCTGCCAATTGTCTTTGCGGCCGCAAC |
| | | GTCCAGGCTGAAAGAACTTTGGCCACCT |
| | | GGCACTGAGAACTGAATTCCATAGGCTG |
| shRNA3 | hsa-miR-146b | TGAGCTCTAGCAATGCCCTGTGGACTCA |
| | | GTTCTGGTGCCCGGCAGTGCTACAACAT |
| | | CAATGCCAAGG (SEQ ID NO: 266) |

TABLE 7
miRNA Sequences

| | Promoted | | SEQ ID |
|---|---|---|---|
| Name | Phenotype | Guide Sequence | NO: |
| hsa-let-7c | M2 | GCAUCCGGGUUGAGGUAGUAGGUUGUAUGG | 267 |
| | | UUUAGAGUUACACCCUGGGAGUUAACUGUA | |
| | | CAACCUUCUAGCUUUCCUUGGAGC | |
| hsa-miR-124 | M2 | AGGCCUCUCUCUCCGUGUUCACAGCGGACCU | 268 |
| | | UGAUUUAAAUGUCCAUACAAUUAAGGCACG | |
| | | CGGUGAAUGCCAAGAAUGGGGCUG | |
| hsa-miR-125a | M1 | UGCCAGUCUCUAGGUCCCUGAGACCCUUUAA | 269 |
| | | CCUGUGAGGACAUCCAGGGUCACAGGUGAG | |
| | | GUUCUUGGGAGCCUGGCGUCUGGCC | |
| hsa-miR-125b | M1 | UGCGCUCCUCUCAGUCCCUGAGACCCUAACU | 270 |
| | | UGUGAUGUUUACCGUUUAAAUCCACGGGUU | |
| | | AGGCUCUUGGGAGCUGCGAGUCGUGCU | |
| hsa-miR-130a | M1 | UGCUGCUGGCCAGAGCUCUUUUCACAUUGU | 271 |
| | | GCUACUGUCUGCACCUGUCACUAGCAGUGCA | |
| | | AUGUUAAAAGGGCAUUGGCCGUGUAGUG | |
| hsa-miR-130b | M1 | GGCCUGCCCGACACUCUUUCCCUGUUGCACU | 272 |
| | | ACUAUAGGCCGCUGGGAAGCAGUGCAAUGA | |
| | | UGAAAGGGCAUCGGUCAGGUC | |
| hsa-miR-142 | M2 | GACAGUGCAGUCACCCAUAAAGUAGAAAGC | 273 |
| | | ACUACUAACAGCACUGGAGGGUGUAGUGUU | |
| | | UCCUACUUUAUGGAUGAGUGUACUGUG | |
| hsa-miR-145 | M2 | CACCUUGUCCUCACGGUCCAGUUUUCCCAGG | 274 |
| | | AAUCCCUUAGAUGCUAAGAUGGGGAUUCCU | |
| | | GGAAAUACUGUUCUUGAGGUCAUGGUU | |
| hsa-miR-146a | M2 | CCGAUGUGUAUCCUCAGCUUUGAGAACUGA | 275 |
| | | AUUCCAUGGGUUGUGUCAGUGUCAGACCUC | |
| | | UGAAAUUCAGUUCUUCAGCUGGGAUAUCUC | |
| | | UGUCAUCGU | |
| hsa-miR-146b | M2 | CCUGGCACUGAGAACUGAAUUCCAUAGGCU | 276 |
| | | GUGAGCUCUAGCAAUGCCCUGUGGACUCAG | |
| | | UUCUGGUGCCCGG | |
| hsa-miR-155 | M1 | CUGUUAAUGCUAAUCGUGAUAGGGGUUUUU | 277 |
| | | GCCUCCAACUGACUCCUACAUAUUAGCAUUA | |
| | | ACAG | |
| hsa-miR-181a | M2 | UGAGUUUUGAGGUUGCUUCAGUGAACAUUC | 278 |
| | | AACGCUGUCGGUGAGUUUGGAAUUAAAAUC | |
| | | AAAACCAUCGACCGUUGAUUGUACCCUAUG | |
| | | GCUAACCAUCAUCUACUCCA | |
| hsa-miR-193 | M2 | CGAGGAUGGGAGCUGAGGGCUGGGUCUUUG | 279 |
| | | CGGGCGAGAUGAGGGUGUCGGAUCAACUGG | |
| | | CCUACAAAGUCCCAGUUCUCGGCCCCCG | |
| hsa-miR-19a-3p | M1 | GCAGUCCUCUGUUAGUUUUGCAUAGUUGCA | 280 |
| | | CUACAAGAAGAAUGUAGUUGUGCAAAUCUA | |
| | | UGCAAAACUGAUGGUGGCCUGC | |
| hsa-miR-21 | M1 | UGUCGGGUAGCUUAUCAGACUGAUGUUGAC | 281 |
| | | UGUUGAAUCUCAUGGCAACACCAGUCGAUG | |
| | | GGCUGUCUGACA | |
| hsa-miR-210 | M2 | ACCCGGCAGUGCCUCCAGGCGCAGGGCAGCC | 282 |
| | | CCUGCCCACCGCACACUGCGCUGCCCCAGAC | |
| | | CCACUGUGCGUGUGACAGCGGCUGAUCUGU | |
| | | GCCUGGGCAGCGCGACCC | |
| hsa-miR-223 | M2 | CCUGGCCUCCUGCAGUGCCACGCUCCGUGUA | 283 |
| | | UUUGACAAGCUGAGUUGGACACUCCAUGUG | |
| | | GUAGAGUGUCAGUUUGUCAAAUACCCCAAG | |
| | | UGCGGCACAUGCUUACCAG | |
| hsa-miR-23a | M1 | GGCCGGCUGGGGUUCCUGGGGAUGGGAUUU | 284 |
| | | GCUUCCUGUCACAAAUCACAUUGCCAGGGAU | |
| | | UUCCAACCGACC | |
| hsa-miR-24 | M2 | CUCUGCCUCCCGUGCCUACUGAGCUGAAACA | 285 |
| | | CAGUUGGUUUGUGUACACUGGCUCAGUUCA | |
| | | GCAGGAACAGGG | |
| hsa-miR-26a | M1 | GUGGCCUCGUUCAAGUAAUCCAGGAUAGGC | 286 |
| | | UGUGCAGGUCCCAAUGGGCCUAUUCUUGGU | |
| | | UACUUGCACGGGGACGC | |
| hsa-miR-26b | M1 | CCGGGACCCAGUUCAAGUAAUUCAGGAUAG | 287 |
| | | GUUGUGUGCUGUCCAGCCUGUUCUCCAUUAC | |
| | | UUGGCUCGGGGACCGG | |
| hsa-miR-27a | M1 | CUGAGGAGCAGGGCUUAGCUGCUUGUGAGC | 288 |
| | | AGGGUCCACACCAAGUCGUGUUCACAGUGGC | |
| | | UAAGUUCCGCCCCCCAG | |
| hsa-miR-27b | M1 | ACCUCUCUAACAAGGUGCAGAGCUUAGCUG | 289 |
| | | AUUGGUGAACAGUGAUUGGUUUCCGCUUUG | |
| | | UUCACAGUGGCUAAGUUCUGCACCUGAAGA | |
| | | GAAGGUG | |
| hsa-miR-30a | M2 | GCGACUGUAAACAUCCUCGACUGGAAGCUG | 290 |
| | | UGAAGCCACAGAUGGGCUUUCAGUCGGAUG | |
| | | UUUGCAGCUGC | |
| hsa-miR-30b | M2 | ACCAAGUUUCAGUUCAUGUAAACAUCCUAC | 291 |
| | | ACUCAGCUGUAAUACAUGGAUUGGCUGGGA | |
| | | GGUGGAUGUUUACUUCAGCUGACUUGGA | |
| hsa-miR-378a | M2 | AGGGCUCCUGACUCCAGGUCCUGUGUGUUAC | 292 |
| | | CUAGAAAUAGCACUGGACUUGGAGUCAGAA | |
| | | GGCCU | |
| hsa-miR-511 | M2 | CAAUAGACACCCAUCGUGUCUUUUGCUCUGC | 293 |
| | | AGUCAGUAAAUAUUUUUUUGUGAAUGUGUA | |
| | | GCAAAAGACAGAAUGGUGGUCCAUUG | |
| hsa-miR-9 | M2 | CGGGGUUGGUUGUUAUCUUUGGUUAUCUAG | 294 |
| | | CUGUAUGAGUGGUGUGGAGUCUUCAUAAAG | |
| | | CUAGAUAACCGAAAGUAAAAAUAACCCCA | |
| hsa-miR-93 | M2 | CUGGGGGCUCCAAAGUGCUGUUCGUGCAGG | 295 |
| | | UAGUGUGAUUACCCAACCUACUGCUGAGCU | |
| | | AGCACUUCCCGAGCCCCCGG | |

TABLE 8
Target Nucleic Acid Sequences

| Name/Gene | |
|---|---|
| Symbol | |
| Transcript | |
| Gene ID | mRNA Sequence |
| SIRPA | UCCGGCCCGCACCCACCCCCAAGAGGGGCCUUCAGCUUUGGGGCUCAGAGG |
| NM_ | CACGACCUCCUGGGGAGGGUUAAAAGGCAGACGCCCCCCCGCCCCCCGCGC |
| 001040022 | CCCCGCGCCCCGACUCCUUCGCCGCCUCCAGCCUCUCGCCAGUGGGAAGCG |
| 140885 | GGGAGCAGCCGCGCGGCCGGAGUCCGGAGGCGAGGGGAGGUCGGCCGCAA |
| | CUUCCCCGGUCCACCUUAAGAGGACGAUGUAGCCAGCUCGCAGCGCUGAC |
| | CUUAGAAAAACAAGUUUGCGCAAAGUGGAGCGGGGACCCGGCCUCUGGGC |
| | AGCCCCGGCGGCGCUUCCAGUGCCUUCCAGCCCUCGCGGGCGGCGCAGCCG |
| | CGGCCCAUGGAGCCCGCCGGCCCGGCCCCCGGCCGCCUCGGGCCGCUGCUC |
| | UGCCUGCUGCUCGCCGCGUCCUGCGCCUGGUCAGGAGUGGCGGGUGAGGA |
| | GGAGCUGCAGGUGAUUCAGCCUGACAAGUCCGUGUUGGUUGCAGCUGGAG |
| | AGACAGCCACUCUGCGCUGCACUGCGACCUCUCUGAUCCCUGUGGGGCCCA |
| | UCCAGUGGUUCAGAGGAGCUGGACCAGGCCGGGAAUUAAUCUACAAUCAA |
| | AAAGAAGGCCACUUCCCCCGGGUAACAACUGUUUCAGACCUCACAAAGAG |
| | AAACAACAUGGACUUUUCCAUCCGCAUCGGUAACAUCACCCCAGCAGAUG |
| | CCGGCACCUACUACUGUGUGAAGUUCCGGAAAGGGAGCCCCGAUGACGUG |
| | GAGUUUAAGUCUGGAGCAGGCACUGAGCUGUCUGUGCGCGCCAAACCCUC |
| | UGCCCCCGUGGUAUCGGGCCCUGCGGCGAGGGCCACACCUCAGCACACAGU |
| | GAGCUUCACCUGCGAGUCCCACGGCUUCUCACCCAGAGACAUCACCCUGAA |
| | AUGGUUCAAAAAUGGGAAUGAGCUCUCAGACUUCCAGACCAACGUGGACC |
| | CCGUAGGAGAGAGCGUGUCCUACAGCAUCCACAGCACAGCCAAGGUGGUG |
| | CUGACCCGCGAGGACGUUCACUCUCAAGUCAUCUGCGAGGUGGCCCACGU |
| | CACCUUGCAGGGGGACCCUCUUCGUGGGACUGCCAACUUGUCUGAGACCA |
| | UCCGAGUUCCACCCACCUUGGAGGUUACUCAACAGCCCGUGAGGGCAGAG |
| | AACCAGGUGAAUGUCACCUGCCAGGUGAGGAAGUUCUACCCCCAGAGACU |
| | ACAGCUGACCUGGUUGGAGAAUGGAAACGUGUCCCGGACAGAAACGGCCU |
| | CAACCGUUACAGAGAACAAGGAUGGUACCUACAACUGGAUGAGCUGGCUC |
| | CUGGUGAAUGUAUCUGCCCACAGGGAUGAUGUGAAGCUCACCUGCCAGGU |
| | GGAGCAUGACGGGCAGCCAGCGGUCAGCAAAAGCCAUGACCUGAAGGUCU |
| | CAGCCCACCCGAAGGAGCAGGGCUCAAAUACCGCCGCUGAGAACACUGGA |
| | UCUAAUGAACGGAACAUCUAUAUUGUGGUGGGUGUGGUGUGCACCUUGCU |
| | GGUGGCCCUACUGAUGGCGGCCCUCUACCUCGUCCGAAUCAGACAGAAGA |
| | AAGCCCAGGGCUCCACUUCUUCUACAAGGUUGCAUGAGCCCGAGAAGAAU |
| | GCCAGAGAAAUAACACAGGACACAAAUGAUAUCACAUAUGCAGACCUGAA |
| | CCUGCCCAAGGGGAAGAAGCCUGCUCCCCAGGCUGCGGAGCCCAACAACCA |
| | CACGGAGUAUGCCAGCAUUCAGACCAGCCCGCAGCCCGCGUCGGAGGACA |
| | CCCUCACCUAUGCUGACCUGGACAUGGUCCACCUCAACCGGACCCCCAAGC |
| | AGCCGGCCCCCAAGCCUGAGCCGUCCUUCUCAGAGUACGCCAGCGUCCAGG |
| | UCCCGAGGAAGUGAAUGGGACCGUGGUUUGCUCUAGCACCCAUCUCUACG |
| | CGCUUUCUUGUCCCACAGGGAGCCGCCGUGAUGAGCACAGCCAACCCAGU |
| | UCCCGGAGGGCUGGGGCGGUGCAGGCUCUGGGACCCAGGGGCCAGGGUGG |
| | CUCUUCUCUCCCCACCCCUCCUUGGCUCUCCAGCACUUCCUGGGCAGCCAC |
| | GGCCCCCUCCCCCCACAUUGCCACAUACCUGGAGGCUGACGUUGCCAAACC |
| | AGCCAGGGAACCAACCUGGGAAGUGGCCAGAACUGCCUGGGGUCCAAGAA |
| | CUCUUGUGCCUCCGUCCAUCACCAUGUGGGUUUUGAAGACCCUCGACUGC |
| | CUCCCCGAUGCUCCGAAGCCUGAUCUUCCAGGGUGGGGAGGAGAAAAUCC |
| | CACCUCCCCUGACCUCCACCACCUCCACCACCACCACCACCACCACCACCAC |
| | CACUACCACCACCACCCAACUGGGGCUAGAGUGGGGAAGAUUUCCCCUUU |
| | AGAUCAAACUGCCCCUUCCAUGGAAAAGCUGGAAAAAAACUCUGGAACCC |
| | AUAUCCAGGCUUGGUGAGGUUGCUGCCAACAGUCCUGGCCUCCCCCAUCC |
| | CUAGGCUAAAGAGCCAUGAGUCCUGGAGGAGGAGAGGACCCCUCCCAAAG |
| | GACUGGAGACAAAACCCUCUGCUUCCUUGGGUCCCUCCAAGACUCCCUGG |
| | GGCCCAACUGUGUUGCUCCACCCGGACCCAUCUCUCCCUUCUAGACCUGAG |
| | CUUGCCCCUCCAGCUAGCACUAAGCAACAUCUCGCUGUGGACGCCUGUAA |
| | AUUACUGAGAAAUGUGAAACGUGCAAUCUUGAAACUGAGGUGUUAGAAA |
| | ACUUGAUCUGUGGUGUUUUGUUUUGUUUUUUUUCUUAAAACAACAGCAAC |
| | GUGAUCUUGGCUGUCUGUCAUGUGUUGAAGUCCAUGGUUGGGUCUUGUGA |
| | AGUCUGAGGUUUAACAGUUUGUUGUCCUGGAGGGAUUUUCUUACAGCGAA |
| | GACUUGAGUUCCUCCAAGUCCCAGAACCCCAAGAAUGGGCAAGAAGGAUC |
| | AGGUCAGCCACUCCCUGGAGACACAGCCUUCUGGCUGGGACUGACUUGGC |
| | CAUGUUCUCAGCUGAGCCACGCGGCUGGUAGUGCAGCCUUCUGUGACCCC |
| | GCUGUGGUAAGUCCAGCCUGCCCAGGGCUGCUGAGGGCUGCCUCUUGACA |
| | GUGCAGUCUUAUCGAGACCCAAUGCCUCAGUCUGCUCAUCCGUAAAGUGG |
| | GGAUAGUGAAGAUGACACCCCUCCCCACCACCUCUCAUAAGCACUUUAGG |
| | AACACACAGAGGGUAGGGAUAGUGGCCCUGGCCGUCUAUCCUACCCCUUU |
| | AGUGACCGCCCCCAUCCCGGCUUUCUGAGCUGAUCCUUGAAGAAGAAAUC |
| | UUCCAUUUCUGCUCUCAAACCCUACUGGGAUCAAACUGGAAUAAAUUGAA |
| | GACAGCCAGGGGGAUGGUGCAGCUGUGAAGCUCGGGCUGAUUCCCCCUCU |
| | GUCCCAGAAGGUUGGCCAGAGGGUGUGACCCAGUUACCCUUUAACCCCCA |
| | CCCUUCCAGUCGGGUGUGAGGGCCUGACCGGGCCCAGGGCAAGCAGAUGU |
| | CGCAAGCCCUAUUUAUUCAGUCUUCACUAUAACUCUUAGAGUUGAGACGC |
| | UAAUGUUCAUGACUCCUGGCCUUGGGAUGCCCAAGGGAUUUCUGGCUCAG |
| | GCUGUAAAAGUAGCUGAGCCAUCCUGCCCAUUCCUGGAGGUCCUACAGGU |
| | GAAACUGCAGGAGCUCAGCAUAGACCCAGCUCUCUGGGGGAUGGUCACCU |
| | GGUGAUUUCAAUGAUGGCAUCCAGGAAUUAGCUGAGCCAACAGACCAUGU |
| | GGACAGCUUUGGCCAGAGCUCCCGUGUGGCAUCUGGGAGCCACAGUGACC |
| | CAGCCACCUGGCUCAGGCUAGUUCCAAAUUCCAAAAGAUUGGCUUGUAAA |
| | CCUUCGUCUCCCUCUCUUUUACCCAGAGACAGCACAUACGUGUGCACACGC |
| | AUGCACACACACAUUCAGUAUUUUAAAAGAAUGUUUUCUUGGUGCCAUUU |
| | UCAUUUUAUUUUAUUUUUUAAUUCUUGGAGGGGGAAAUAAGGGAAUAAG |
| | GCCAAGGAAGAUGUAUAGCUUUAGCUUUAGCCUGGCAACCUGGAGAAUCC |
| | ACAUACCUUGUGUAUUGAACCCCAGGAAAAGGAAGAGGUCGAACCAACCC |
| | UGCGGAAGGAGCAUGGUUUCAGGAGUUUAUUUUAAGACUGCUGGGAAGG |
| | AAACAGGCCCCAUUUUGUAUAUAGUUGCAACUUAAACUUUUUGGCUUGCA |
| | AAAUAUUUUUGUAAUAAAGAUUUCUGGGUAAUAAUGA (SEQ ID NO: 296) |
| STAT6 | GGGGCAGCCACUGCUUACACUGAAGAGGGAGGACGGGAGAGGAGUGUGUG |
| NM_003153 | UGUGUGUGUGUGUGUGUGUGUGUAUGUAUGUGUGUGCUUUAUCUUAUUU |
| 6778 | UUCUUUUUGGUGGUGGUGGUGGAAGGGGGGAGGUGCUAGCAGGGCCAGCC |
| | UUGAACUCGCUGGACAGAGCUACAGACCUAUGGGGCCUGGAAGUGCCCGC |
| | UGAGAAAGGGAGAAGACAGCAGAGGGGUUGCCGAGGCAACCUCCAAGUCC |
| | CAGAUCAUGUCUCUGUGGGGUCUGGUCUCCAAGAUGCCCCCAGAAAAAGU |
| | GCAGCGGCUCUAUGUCGACUUUCCCCAACACCUGCGGCAUCUUCUGGGUG |
| | ACUGGCUGGAGAGCCAGCCCUGGGAGUUCCUGGUCGGCUCCGACGCCUUC |
| | UGCUGCAACUUGGCUAGUGCCCUACUUUCAGACACUGUCCAGCACCUUCA |
| | GGCCUCGGUGGGAGAGCAGGGGGAGGGGAGCACCAUCUUGCAACACAUCA |
| | GCACCCUUGAGAGCAUAUAUCAGAGGGACCCCCUGAAGCUGGUGGCCACU |
| | UUCAGACAAAUACUUCAAGGAGAGAAAAAAGCUGUUAUGGAACAGUUCCG |
| | CCACUUGCCAAUGCCUUUCCACUGGAAGCAGGAAGAACUCAAGUUUAAGA |
| | CAGGCUUGCGGAGGCUGCAGCACCGAGUAGGGGAGAUCCACCUUCUCCGA |
| | GAAGCCCUGCAGAAGGGGGCUGAGGCUGGCCAAGUGUCUCUGCACAGCUU |
| | GAUAGAAACUCCUGCUAAUGGGACUGGGCCAAGUGAGGCCCUGGCCAUGC |
| | UACUGCAGGAGACCACUGGAGAGCUAGAGGCAGCCAAAGCCCUAGUGCUG |
| | AAGAGGAUCCAGAUUUGGAAACGGCAGCAGCAGCUGGCAGGGAAUGGCGC |
| | ACCGUUUGAGGAGAGCCUGGCCCCACUCCAGGAGAGGUGUGAAAGCCUGG |
| | UGGACAUUUAUUCCCAGCUACAGCAGGAGGUAGGGGCGGCUGGUGGGGAG |
| | CUUGAGCCCAAGACCCGGGCAUCGCUGACUGGCCGGCUGGAUGAAGUCCU |
| | GAGAACCCUCGUCACCAGUUGCUUCCUGGUGGAGAAGCAGCCCCCCCAGG |
| | UACUGAAGACUCAGACCAAGUUCCAGGCUGGAGUUCGAUUCCUGUUGGGC |
| | UUGAGGUUCCUGGGGGCCCCAGCCAAGCCUCCGCUGGUCAGGGCCGACAU |
| | GGUGACAGAGAAGCAGGCGCGGGAGCUGAGUGUGCCUCAGGGUCCUGGGG |
| | CUGGAGCAGAAAGCACUGGAGAAAUCAUCAACAACACUGUGCCCUUGGAG |
| | AACAGCAUUCCUGGGAACUGCUGCUCUGCCCUGUUCAAGAACCUGCUUCU |
| | CAAGAAGAUCAAGCGGUGUGAGCGGAAGGGCACUGAGUCUGUCACAGAGG |
| | AGAAGUGCGCUGUGCUCUUCUCUGCCAGCUUCACACUUGGCCCCGGCAAA |
| | CUCCCCAUCCAGCUCCAGGCCCUGUCUCUGCCCCUGGUGGUCAUCGUCCAU |
| | GGCAACCAAGACAACAAUGCCAAAGCCACUAUCCUGUGGGACAAUGCCUU |
| | CUCUGAGAUGGACCGCGUGCCCUUUGUGGUGGCUGAGCGGGUGCCCUGGG |
| | AGAAGAUGUGUGAAACUCUGAACCUGAAGUUCAUGGCUGAGGUGGGGACC |
| | AACCGGGGGCUGCUCCCAGAGCACUUCCUCUUCCUGGCCCAGAAGAUCUU |
| | CAAUGACAACAGCCUCAGUAUGGAGGCCUUCCAGCACCGUUCUGUGUCCU |
| | GGUCGCAGUUCAACAAGGAGAUCCUGCUGGGCCGUGGCUUCACCUUUUGG |
| | CAGUGGUUUGAUGGUGUCCUGGACCUCACCAAACGCUGUCUCCGGAGCUA |
| | CUGGUCUGACCGGCUGAUCAUUGGCUUCAUCAGCAAACAGUACGUUACUA |
| | GCCUUCUUCUCAAUGAGCCCGACGGAACCUUUCUCCUCCGCUUCAGCGACU |
| | CAGAGAUUGGGGGCAUCACCAUUGCCCAUGUCAUCCGGGGCCAGGAUGGC |
| | UCUCCACAGAUAGAGAACAUCCAGCCAUUCUCUGCCAAAGACCUGUCCAU |
| | UCGCUCACUGGGGGACCGAAUCCGGGAUCUUGCUCAGCUCAAAAAUCUCU |
| | AUCCCAAGAAGCCCAAGGAUGAGGCUUUCCGGAGCCACUACAAGCCUGAA |
| | CAGAUGGGUAAGGAUGGCAGGGGUUAUGUCCCAGCUACCAUCAAGAUGAC |
| | CGUGGAAAGGGACCAACCACUUCCUACCCCAGAGCUCCAGAUGCCUACCA |
| | UGGUGCCUUCUUAUGACCUUGGAAUGGCCCCUGAUUCCUCCAUGAGCAUG |
| | CAGCUUGGCCCAGAUAUGGUGCCCCAGGUGUACCCACCACACUCUCACUCC |
| | AUCCCCCCGUAUCAAGGCCUCUCCCCAGAAGAAUCAGUCAACGUGUUGUC |
| | AGCCUUCCAGGAGCCUCACCUGCAGAUGCCCCCCAGCCUGGGCCAGAUGAG |
| | CCUGCCCUUUGACCAGCCUCACCCCCAGGGCCUGCUGCCGUGCCAGCCUCA |
| | GGAGCAUGCUGUGUCCAGCCCUGACCCCCUGCUCUGCUCAGAUGUGACCA |
| | UGGUGGAAGACAGCUGCCUGAGCCAGCCAGUGACAGCGUUUCCUCAGGGC |
| | ACUUGGAUUGGUGAAGACAUAUUCCCUCCUCUGCUGCCUCCCACUGAACA |
| | GGACCUCACUAAGCUUCUCCUGGAGGGGCAAGGGGAGUCGGGGGGAGGGU |
| | CCUUGGGGGCACAGCCCCUCCUGCAGCCCUCCCACUAUGGGCAAUCUGGGA |
| | UCUCAAUGUCCCACAUGGACCUAAGGGCCAACCCCAGUUGGUGAUCCCAG |
| | CUGGAGGGAGAACCCAAAGAGACAGCUCUUCUACUACCCCCACAGACCUG |
| | CUCUGGACACUUGCUCAUGCCCUGCCAAGCAGCAGAUGGGGAGGGUGCCC |
| | UCCUAUCCCCACCUACUCCUGGGUCAGGAGGAAAAGACUAACAGGAGAAU |
| | GCACAGUGGGUGGAGCCAAUCCACUCCUUCCUUUCUAUCAUUCCCCUGCCC |
| | ACCUCCUUCCAGCACUGACUGGAAGGGAAGUUCAGGCUCUGAGACACACC |
| | CCAACAUGCCUGCACCUGCAGCGCGCACACGCACGCACACACACAUACAGA |
| | GCUCUCUGAGGGUGAUGGGGCUGAGCAGGAGGGGGGCUGGGUAAGAGCAC |
| | AGGUUAGGGCAUGGAAGGCUUCUCCGCCCAUUCUGACCCAGGGCCUAGGA |
| | CGGAUAGGCAGGAACAUACAGACACAUUUACACUAGAGGCCAGGGAUAGA |
| | GGAUAUUGGGUCUCAGCCCUAGGGGAAUGGGAAGCAGCUCAAGGGACCCU |
| | GGGUGGGAGCAUAGGAGGGGUCUGGACAUGUGGUUACUAGUACAGGUUU |
| | UGCCCUGAUUAAAAAAUCUCCCAAAGCCCCAAAUUCCUGUUAGCCAGGUG |
| | GAGGCUUCUGAUACGUGUAUGAGACUAUGCAAAAGUACAAGGGCUGAGAU |
| | UCUUCGUGUAUAGCUGUGUGAACGUGUAUGUACCUAGGAUAUGUUAAAUG |
| | UAUAGCUGGCACCUUAGUUGCAUGACCACAUAGAACAUGUGUCUAUCUGC |
| | UUUUGCCUACGUGACAACACAAAUUUGGGAGGGUGAGACACUGCACAGAA |
| | GACAGCAGCAAGUGUGCUGGCCUCUCUGACAUAUGCUAACCCCCAAAUAC |
| | UCUGAAUUUGGAGUCUGACUGUGCCCAAGUGGGUCCAAGUGGCUGUGACA |
| | UCUACGUAUGGCUCCACACCUCCAAUGCUGCCUGGGAGCCAGGGUGAGAG |
| | UCUGGGUCCAGGCCUGGCCAUGUGGCCCUCCAGUGUAUGAGAGGGCCCUG |
| | CCUGCUGCAUCUUUUCUGUUGCCCCAUCCACCGCCAGCUUCCCUUCACUCC |
| | CCUAUCCCAUUCUCCCUCUCAAGGCAGGGGUCAUAGAUCCUAAGCCAUAA |
| | AAUAAAUUUUAUUCCAAAAUAACAAAAUAAAUAAUCUACUGUACACAAUC |
| | UGAAAA (SEQ ID NO: 297) |
| PIK3CG | GCACUUCCUUCUCGGCUAGAUUAUCUGAAACUGUUGUCGGUUCUUGAGAU |
| NM_002649 | GAUACUACCACCGAAUGUCUGUGUUUCAUUGUCUAGUCCAACCUGUAUUG |
| 5294 | UGGAUAUCUACAACGUUCCGGCAAUAGUUUUGCAGGUGCAUCACAUUUUU |
| | GUUUUUGUUUUGGGAGGAAAAGGGAGGGCACGGCAGCCAGGCUUCAUAUU |
| | CCUACAAGUGCAUGCUUCAAGAUUACUGUACUUACAGUGUUUCCAACAUC |
| | UUCUCAUAAAAGGGGAAAGCUUCAUAGCCUCAACCAUGAAGGAAACCAGU |
| | CGCAUAGGGCAUGGAGCUGGAGAACUAUAAACAGCCCGUGGUGCUGAGAG |
| | AGGACAACUGCCGAAGGCGCCGGAGGAUGAAGCCGCGCAGUGCUGCGGCC |
| | AGCCUGUCCUCCAUGGAGCUCAUCCCCAUCGAGUUCGUGCUGCCCACCAGC |
| | CAGCGCAAAUGCAAGAGCCCCGAAACGGCGCUGCUGCACGUGGCCGGCCA |
| | CGGCAACGUGGAGCAGAUGAAGGCCCAGGUGUGGCUGCGAGCGCUGGAGA |
| | CCAGCGUGGCGGCGGACUUCUACCACCGGCUGGGACCGCAUCACUUCCUCC |
| | UGCUCUAUCAGAAGAAGGGGCAGUGGUACGAGAUCUACGACAAGUACCAG |
| | GUGGUGCAGACUCUGGACUGCCUGCGCUACUGGAAGGCCACGCACCGGAG |
| | CCCGGGCCAGAUCCACCUGGUGCAGCGGCACCCGCCCUCCGAGGAGUCCCA |
| | AGCCUUCCAGCGGCAGCUCACGGCGCUGAUUGGCUAUGACGUCACUGACG |
| | UCAGCAACGUGCACGACGAUGAGCUGGAGUUCACGCGCCGUGGCUUGGUG |
| | ACCCCGCGCAUGGCGGAGGUGGCCAGCCGCGACCCCAAGCUCUACGCCAUG |
| | CACCCGUGGGUGACGUCCAAGCCCCUCCCGGAGUACCUGUGGAAGAAGAU |
| | UGCCAACAACUGCAUCUUCAUCGUCAUUCACCGCAGCACCACCAGCCAGAC |
| | CAUUAAGGUCUCACCCGACGACACCCCCGGCGCCAUCCUGCAGAGCUUCUU |
| | CACCAAGAUGGCCAAGAAGAAAUCUCUGAUGGAUAUUCCCGAAAGCCAAA |
| | GCGAACAGGAUUUUGUGCUGCGCGUCUGUGGCCGGGAUGAGUACCUGGUG |
| | GGCGAAACGCCCAUCAAAAACUUCCAGUGGGUGAGGCACUGCCUCAAGAA |
| | CGGAGAAGAGAUUCACGUGGUACUGGACACGCCUCCAGACCCGGCCCUAG |
| | ACGAGGUGAGGAAGGAAGAGUGGCCACUGGUGGAUGACUGCACGGGAGUC |
| | ACCGGCUACCAUGAGCAGCUUACCAUCCACGGCAAGGACCACGAGAGUGU |
| | GUUCACCGUGUCCCUGUGGGACUGCGACCGCAAGUUCAGGGUCAAGAUCA |
| | GAGGCAUUGAUAUCCCCGUCCUGCCUCGGAACACCGACCUCACAGUUUUU |
| | GUAGAGGCAAACAUCCAGCAUGGGCAACAAGUCCUUUGCCAAAGGAGAAC |
| | CAGCCCCAAACCCUUCACAGAGGAGGUGCUGUGGAAUGUGUGGCUUGAGU |
| | UCAGUAUCAAAAUCAAAGACUUGCCCAAAGGGGCUCUACUGAACCUCCAG |
| | AUCUACUGCGGUAAAGCUCCAGCACUGUCCAGCAAGGCCUCUGCAGAGUC |
| | CCCCAGUUCUGAGUCCAAGGGCAAAGUUCAGCUUCUCUAUUAUGUGAACC |
| | UGCUGCUGAUAGACCACCGUUUCCUCCUGCGCCGUGGAGAAUACGUCCUC |
| | CACAUGUGGCAGAUAUCUGGGAAGGGAGAAGACCAAGGAAGCUUCAAUGC |
| | UGACAAACUCACGUCUGCAACUAACCCAGACAAGGAGAACUCAAUGUCCA |
| | UCUCCAUUCUUCUGGACAAUUACUGCCACCCGAUAGCCCUGCCUAAGCAU |
| | CAGCCCACCCCUGACCCGGAAGGGGACCGGGUUCGAGCAGAAAUGCCCAA |
| | CCAGCUUCGCAAGCAAUUGGAGGCGAUCAUAGCCACUGAUCCACUUAACC |
| | CUCUCACAGCAGAGGACAAAGAAUUGCUCUGGCAUUUUAGAUACGAAAGC |
| | CUUAAGCACCCAAAAGCAUAUCCUAAGCUAUUUAGUUCAGUGAAAUGGGG |
| | ACAGCAAGAAAUUGUGGCCAAAACAUACCAAUUGUUGGCCAGAAGGGAAG |
| | UCUGGGAUCAAAGUGCUUUGGAUGUUGGGUUAACAAUGCAGCUCCUGGAC |
| | UGCAACUUCUCAGAUGAAAAUGUAAGAGCCAUUGCAGUUCAGAAACUGGA |
| | GAGCUUGGAGGACGAUGAUGUUCUGCAUUACCUUCUACAAUUGGUCCAGG |
| | CUGUGAAAUUUGAACCAUACCAUGAUAGCGCCCUUGCCAGAUUUCUGCUG |
| | AAGCGUGGUUUAAGAAACAAAAGAAUUGGUCACUUUUUGUUUUGGUUCU |
| | UGAGAAGUGAGAUAGCCCAGUCCAGACACUAUCAGCAGAGGUUCGCUGUG |
| | AUUCUGGAAGCCUAUCUGAGGGGCUGUGGCACAGCCAUGCUGCACGACUU |
| | UACCCAACAAGUCCAAGUAAUCGAGAUGUUACAAAAAGUCACCCUUGAUA |
| | UUAAAUCGCUCUCUGCUGAAAAGUAUGACGUCAGUUCCCAAGUUAUUUCA |
| | CAACUUAAACAAAAGCUUGAAAACCUGCAGAAUUCUCAACUCCCCGAAAG |
| | CUUUAGAGUUCCAUAUGAUCCUGGACUGAAAGCAGGAGCGCUGGCAAUUG |
| | AAAAAUGUAAAGUAAUGGCCUCCAAGAAAAAACCACUAUGGCUUGAGUUU |
| | AAAUGUGCCGAUCCUACAGCCCUAUCAAAUGAAACAAUUGGAAUUAUCUU |
| | UAAACAUGGUGAUGAUCUGCGCCAAGACAUGCUUAUUUUACAGAUUCUAC |
| | GAAUCAUGGAGUCUAUUUGGGAGACUGAAUCUUUGGAUCUAUGCCUCCUG |
| | CCAUAUGGUUGCAUUUCAACUGGUGACAAAAUAGGAAUGAUCGAGAUUGU |
| | GAAAGACGCCACGACAAUUGCCAAAAUUCAGCAAAGCACAGUGGGCAACA |
| | CGGGAGCAUUUAAAGAUGAAGUCCUGAAUCACUGGCUCAAAGAAAAAUCC |
| | CCUACUGAAGAAAAGUUUCAGGCAGCAGUGGAGAGAUUUGUUUAUUCCUG |
| | UGCAGGCUACUGUGUGGCAACCUUUGUUCUUGGAAUAGGCGACAGACACA |
| | AUGACAAUAUUAUGAUCACCGAGACAGGAAACCUAUUUCAUAUUGACUUC |
| | GGGCACAUUCUUGGGAAUUACAAAAGUUUCCUGGGCAUUAAUAAAGAGAG |
| | AGUGCCAUUUGUGCUAACCCCUGACUUCCUCUUUGUGAUGGGAACUUCUG |
| | GAAAGAAGACAAGCCCACACUUCCAGAAAUUUCAGGACAUCUGUGUUAAG |
| | GCUUAUCUAGCCCUUCGUCAUCACACAAACCUACUGAUCAUCCUGUUCUC |
| | CAUGAUGCUGAUGACAGGAAUGCCCCAGUUAACAAGCAAAGAAGACAUUG |
| | AAUAUAUCCGGGAUGCCCUCACAGUGGGGAAAAAUGAGGAGGAUGCUAAA |
| | AAGUAUUUUCUUGAUCAGAUCGAAGUUUGCAGAGACAAAGGAUGGACUGU |
| | GCAGUUUAAUUGGUUUCUACAUCUUGUUCUUGGCAUCAAACAAGGAGAGA |
| | AACAUUCAGCCUAAUACUUUAGGCUAGAAUCAAAAACAAGUUAGUGUUCU |
| | AUGGUUUAAAUUAGCAUAGCAAUCAUCGAACUUGGAUUUCAAAUGCAAUA |
| | GACAUUGUGAAAGCUGGCAUUUCAGAAGUAUAGCUCUUUUCCUACCUGAA |
| | CUCUUCCCUGGAGAAAAGAUGUUGGCAUUGCUGAUUGUUUGGUUAAGCAA |
| | UGUCCAGUGCUAGGAUUAUUUGCAGGUUUGGUUUUUUCUCAUUUGUCUGU |
| | GGCAUUGGAGAAUAUUCUCGGUUUAAACAGACUAAUGACUUCCUUAUUGU |
| | CCCUGAUAUUUUGACUAUCUUACUAUUGAGUGCUUCUGGAAAUUCUUUGG |
| | AAUAAUUGAUGACAUCUAUUUUCAUCUGGGUUUAGUCUCAAUUUUGGUUA |
| | UCUUUGUGUUCCUCAAGCUCUUUAAAGAAAAAGAUGUAAUCGUUGUAACC |
| | UUUGUCUCAUUCCUUAAAUGAUGCUUCCAAACAUCUCCUUAGUGUCUGCA |
| | GGUGUUAGUGGUGUGCUAAAAGCAAGGAAAGCGAGUUAGUCUUUUCAGU |
| | GUCUUUUGCAAUUCAAUUCUUUUGUCAUGUAUAACUGAGACACACAAACA |
| | CAGCAGGAGAAAUCUAAACCGUUGUGCCUUGACCUUCCUCUGCUGGUCUU |
| | GUUCCAGGGUUAUGAAUAUGAAAAAAUAGAGAUGAGACUUUUUGUGUCA |
| | ACUCUGUCCACAAGAGUGAGUUAUCUAGUAUGAUUAGUAUAGCUUUCUCC |
| | AGCAUGGCAGCAGGAAGUAACUACAGGGCCUCUUUUAUGCCUGACAUUUC |
| | UUCCCUUCCUUUUUCCCUGCCUCCCUUUUUCAUCAAUUGCGAUGCUCCCAC |
| | AACUCUUUACAGACUUGUGAAAUCUUCAAGAACACCUUUACUCUAUAACU |
| | CAAAAAUUAGUUGAAAAAUAAUUACUUCUCAAGGAUUAUUAGAAUCUUA |
| | GGUACUUAUUUGUAAAGAUGUUUAGUGACUUUUUUUUCAAGUAUCUUAU |
| | UAAAGGAGGCAUUCUAGAAAAUAUGAAUUAGUUUCCAAAUGCCUUAAUUU |
| | UAAACUUUGGCCUGAACAGUUUUUUCUUUUUCUUAAUGGAAGAAGAUAUU |
| | UAAUAUCUUAAAAAUAUUCCAAGUUAGGAAGAACACUACUUGCCUUAUCC |
| | AUUUCCCAUUUAAAGGACUUUUAAACUUUGACACAUCCUUCAGAUUUCCU |
| | GAAAAUAAUUGAAAUAUCUUACUUUAAAAAUAUUUUCAUCUCUGAAAUA |
| | UCUCGUUAUUUAUUGGAGGUAUUGUUUAACCUUAGAGAGACCAUUAAAUU |
| | AUUUAUAAAAUAUUUUGUAAUUACCUGUAGCUAAUACAUUACAUAGAAA |
| | AAAACUAUGUUAACAGUGUCUCUGUUUAAGUAUAAUCAGAUAUAAAUAU |
| | AUACUUAAUUUUUUAAUUUUAAAAAUAGAUACCUGUUUGACUUUGAGGU |
| | AGUCCAGACCUUUUCUUUUUUUUUUUUUUUUUAAUGUGUGCAAAAGCCCA |
| | AAGGUUCCUAAGCCUGGCUGCAAAGAAGAAUCAACAGGGACACUUUUUAA |
| | AAACACUCUUAUCAGCCUGGGCAACACAGUGAGACUCCAUCUCUUAAAAA |
| | AAAAAUUAGCUGGGUAUAGUGGUAUGUGCCUGUAGUCCCAGGUACUCAGG |
| | AGGCUGAGGCAGGAGGAUUGCCUGAGCCCAGGAGGUGGAAACUGCAGAGA |
| | GUCAUGAUCAUGUCCUUACACUCCAGCCUGGAUAACAGAGCGAGACCCUG |
| | UCUCAAAAAAAUAAAAUAAAAAAUAAAAACACCCUUGCCUGCGCUCCAUU |
| | CCCAGGUUAUAAUUUAAUUACUGUGGGAUGAGACCCAGACAUCAAUAUUU |
| | UUCUAGAUUCUUCAGGUGAUUCUAAUUCACAGCCAGAGUUGGAAGCCCUG |
| | CGUGGCCUUUGAAGGUCUAGAUGAUUCUUCUUCCUUGCCCUUUGAGCUUU |
| | UCCCCAUCUCACAGGUAUCUAGAAAAAAACUCCUCUUCUUUGGCAACCUG |
| | UCCUUUUAAAAUCACACUCUACCCACCUGUACAGAAGACCAUGCCCUAUA |
| | AUGAAAUGUUUAUUCCUAUCUAUAAAUGGAGGAUAAACAUUUUGUGGCAC |
| | UUCUGGACCAACUAUUCCCUACUAUUCUUUUGAAGAAAGGCAGGAAGAGU |
| | ACUUUCUAAUUCAGAAGAGGAUGUUUUCACUAUUCUGAUAAACAAUAGCC |
| | AAGUUCAGACCUUGUACAGAUUCUUUUUAUUUGAAUUGCUGAAAUAAUUU |
| | AUUGAUGAUGAAAAAAAGAUUAGAGAGGAAUACAUUUAUAUUUAGCUUA |
| | UUGGCACAUGUGCAUACAUAUUUCCUCUUCAAAUGAACCAGUUCUUUCAU |
| | UUCAUUAUGCUAAUAUAUAUAUAUAACAUAUAUAUGCUAAUAUAUAUAU |
| | AACAUAUAUAUGCUAAUAUAUAUAUAUCACACAUAUAUAUCACAGUUUUA |
| | UAUAUAUAUAUGUAUGUGUGUGUGUAUAUAUAUAUAUAUAUAUCACAGU |
| | UGGGCUUGAUUCUUCCGUAUUCCAAAGAGCAUAAUUUCAGUUCUAUAGAC |
| | UUAUAGAUAAAUAAAAAUCAUCUUUGUGGGCUUCCUUCCUUUACUGUUCG |
| | CAGUGAAUUACAUGACGAACAACUUCUAUACCUUUGAAAAUGUUCUAGAA |
| | CUAGAAUCAUCCUGCUACUGGGAACUACCCACAGCUCUAUCUUCAAUGCC |
| | AGGUGAAAACACAGAUCACAAGUCAGAUGAAUCAGGCCAAAGCAACUUUU |
| | AUGUAUAUCUAGGACUGGCGACAUAAUUUGCAGAGCCCAGUGAACAGUGA |
| | AAAUGCAGUCCCAUCAUUCAAAAAUUAUUAAGAAAUUCGAGAUGGCAACA |
| | GCAGUUGUCACACCAAGCACAGUGCCCUUCUGAGCAUGAGGCUCUGUGUG |
| | ACUGCAUGGGUCCAUGAGACCAGCACUGUGUGGGUCUGAAUGAUGGCUUU |
| | GGUGUUCAGUUUAGCACACGCGGUCUACCACGUCUGCAUGAGUGGUAAAU |
| | GUAGUGCCUGGGUCAAAAUGUUCCUUCUGUUCAAAUCGAAAUACCUCAUC |
| | UCUAUGGCUCUAUGGCUGUACAUUAGGACCUAGAACAGUGGCCCAUUGCU |
| | CUUAGACUGGAACCAUGUCCACUAAAAUAAACCUAAGCAGAUGUUGUAGA |
| | CCUAGCCCCACAGGACUGCAUUUAGCUGCUUCAGUGACACUUUGAUGAAA |
| | GUAUGGAGAAGUGGAGACAUUAUAGAUAAAAUAUAUCAAUUCCCAGAGA |
| | AAACUCUUGACUUAAAAACUUAACUGUAGUAAAUAUAUCUUUUUCAGGUG |
| | AUGAAUUAUUUUUUUAAAAAAGGUUACAUAUAGGAAUUCUGCAGUAUAA |
| | UUUGGAGGCUAUUAGUGCUAUAUUAAUGGAAAUUAAUUAUUUUUUAAGU |
| | AAGUCCAAAAAAUAAUCUAGAAAGUAAGUUUCCAGAGCAAAUCUGACCUA |
| | GCAUUUGGUAUGCUAGGCUCUGCUUUUCAUGAUUUUGAAAUAAAUCAUAA |
| | UUAGACUUAACAAUAUGGAGAAAAUAAACUUGUAUUUUUAAGUGUUCUG |
| | UUGGCUUAUUUUCUGUUUCAUCCAACUCAAUAAUUCUGAUAAAUAAAUUU |
| | GGUUCUAGUUUGGUGCUUGAAAAAAAA (SEQ ID NO: 298) |
| CLECIA | ACAGUAGCCCGGCGGCCCAGGGCAAUCCGACCACAUUUCACUCUCACCGCU |
| NM_ | GUAGGAAUCCAGAUGCAGGCCAAGUACAGCAGCACGAGGGACAUGCUGGA |
| 001297748 | UGAUGAUGGGGACACCACCAUGAGCCUGCAUUCUCAAGGCUCUGCCACAA |
| 51267 | CUCGGCAUCCAGAGCCCCGGCGCACAGUUUUUCAGUACUACCAGCUCUCCA |
| | AUACUGGUCAAGACACCAUUUCUCAAAUGGAAGAAAGAUUAGGAAAUACG |
| | UCCCAAGAGUUGCAAUCUCUUCAAGUCCAGAAUAUAAAGCUUGCAGGAAG |
| | UCUGCAGCAUGUGGCUGAAAAACUCUGUCGUGAGCUGUAUAACAAAGCUG |
| | GAGCACACAGGUGCAGCCCUUGUACAGAACAAUGGAAAUGGCAUGGAGAC |
| | AAUUGCUACCAGUUCUAUAAAGACAGCAAAAGUUGGGAGGACUGUAAAUA |
| | UUUCUGCCUUAGUGAAAACUCUACCAUGCUGAAGAUAAACAAACAAGAAG |
| | ACCUGGAAUUUGCCGCGUCUCAGAGCUACUCUGAGUUUUUCUACUCUUAU |
| | UGGACAGGGCUUUUGCGCCCUGACAGUGGCAAGGCCUGGCUGUGGAUGGA |
| | UGGAACCCCUUUCACUUCUGAACUGUUCCAUAUUAUAAUAGAUGUCACCA |
| | GCCCAAGAAGCAGAGACUGUGUGGCCAUCCUUAAUGGGAUGAUCUUCUCA |
| | AAGGACUGCAAAGAAUUGAAGCGUUGUGUCUGUGAGAGAAGGGCAGGAA |
| | UGGUGAAGCCAGAGAGCCUCCAUGUCCCCCCUGAAACAUUAGGCGAAGGU |
| | GACUGAUUCGCCCUCUGCAACUACAAAUAGCAGAGUGAGCCAGGCGGUGC |
| | CAAAGCAAGGGCUAGUUGAGACAUUGGGAAAUGGAACAUAAUCAGGAAAG |
| | ACUAUCUCUCUGACUAGUACAAAAUGGGUUCUCGUGUUUCCUGUUCAGGA |
| | UCACCAGCAUUUCUGAGCUUGGGUUUAUGCACGUAUUUAACAGUCACAAG |
| | AAGUCUUAUUUACAUGCCACCAACCAACCUCAGAAACCCAUAAUGUCAUC |
| | UGCCUUCUUGGCUUAGAGAUAACUUUUAGCUCUCUUUCUUCUCAAUGUCU |
| | AAUAUCACCUCCCUGUUUUCAUGUCUUCCUUACACUUGGUGGAAUAAGAA |
| | ACUUUUUGAAGUAGAGGAAAUACAUUGAGGUAACAUCCUUUUCUCUGACA |
| | GUCAAGUAGUCCAUCAGAAAUUGGCAGUCACUUCCCAGAUUGUACCAGCA |
| | AAUACACAAGGAAUUCUUUUUGUUUGUUUCAGUUCAUACUAGUCCCUUCC |
| | CAAUCCAUCAGUAAAGACCCCAUCUGCCUUGUCCAUGCCGUUUCCCAACA |
| | GGGAUGUCACUUGAUAUGAGAAUCUCAAAUCUCAAUGCCUUAUAAGCAUU |
| | CCUUCCUGUGUCCAUUAAGACUCUGAUAAUUGUCUCCCCUCCAUAGGAAU |
| | UUCUCCCAGGAAAGAAAUAUAUCCCCAUCUCCGUUUCAUAUCAGAACUAC |
| | CGUCCCCGAUAUUCCCUUCAGAGAGAUUAAAGACCAGAAAAAAGUGAGCC |
| | UCUUCAUCUGCACCUGUAAUAGUUUCAGUUCCUAUUUUCUUCCAUUGACC |
| | CAUAUUUAUACCUUUCAGGUACUGAAGAUUUAAUAAUAAUAAAUGUAAA |
| | UACUGUGAAGUGUGUGUGAUUUUACAAUGGACUUAUGGUUGGUGGGAAA |
| | AUUCAGCAUGGAAAUGCUUUUCAAAAUAUGAUAGCGGUCAUUAUUUUGAU |
| | UGUGCCUUACUGAAAGUUUUUGGGGAAUUUACAAGAGUACUGAUUACAUG |
| | AUUAUCUGGAGAAAAUAAGAUGUCUUUGAAAUACGUGUUGGCUUCAAGA |
| | AAACAGUUUUAACGUUUUCCUAAAAUGAAAUCUUUUGAGGUGAGCUUAUG |
| | GCAUCAACACAUGGUUGAUGAGGAAGCUGAGUUGCAUUAGUGCACAUGAU |
| | UUCCAGUCAGGUCAUGGGAAAUGAACAGAGACAGUGACAUCUUUGUAGCU |
| | GCUCCUUUGUGAGGCACUUCUUUCUUGAGACGACUCCAUGCACAAAUAUA |
| | ACAGGGAUCAUUGGGAAUGACACCAUCACAGCCACCAAGUUUAUUGGUUU |
| | ACUGAUAAAUCAAUUUUGAGGGUUUUAUGCUAGAAAAGAUAACAUUAUA |
| | AAUCUUUGUUUUUCAACAGUGGAAACUGAGGUGCUUCAGUUUUGUUUUUC |
| | AUAAACAUCUAUCUCAUGGUAAAAUGACGUAUAGUAGCAAUCAUAUGAUA |
| | GAAAGAACACAUUGGUGCUCAUUUUCAUAAACUUCAUUUUGUUAGAUUGU |
| | CACUUAGCAAAGACUAUUGCGGAUUCUUCCUUGAGAACUUUCCUUUCAAU |
| | UUUCUGUUGAUUUCCUUAAAAUUUUCUUCAGGGUCGAGCAAGUUCUGUUG |
| | AUAUAUUGUGUCAUGCAAAGAAAACUGUAUUUGUGGUUAGAAAAUGAAG |
| | AUUUUAGACCUCUUAUUCUGAUUUACUUAUUAGGCAAGUCACUUUACUUC |
| | AUGAAACCUAUUUCACUGCAUAUGAAGUAGAAAUAAUUAUAAUUUCCUCA |
| | UAAUGUUACUGUUAGCUGAAAUAAAGUAAAUGUGAAAUCA (SEQ ID NO: |
| | 299) |
| LILRB2 | GCAUUUCUCCUCUGUGCUCACUGCCACACACAGCUCAACCUGGACAGCACA |
| NM_ | GCCAGAGGCGAGAUGCUUCUCUGCUGAUCUGAGUCUGCCUGCAGCAUGGA |
| 001080978 | CCUGGGUCUUCCCUGAAGCAUCUCCAGGGCUGGAGGGACGACUGCCAUGC |
| 10288 | ACCGAGGGCUCAUCCAUCCGCAGAGCAGGGCAGUGGGAGGAGACGCCAUG |
| | ACCCCCAUCGUCACAGUCCUGAUCUGUCUCGGGCUGAGUCUGGGCCCCAG |
| | GACCCGCGUGCAGACAGGGACCAUCCCCAAGCCCACCCUGUGGGCUGAGCC |
| | AGACUCUGUGAUCACCCAGGGGAGUCCCGUCACCCUCAGUUGUCAGGGGA |
| | GCCUUGAAGCCCAGGAGUACCGUCUAUAUAGGGAGAAAAAAUCAGCAUCU |
| | UGGAUUACACGGAUACGACCAGAGCUUGUGAAGAACGGCCAGUUCCACAU |
| | CCCAUCCAUCACCUGGGAACACACAGGGCGAUAUGGCUGUCAGUAUUACA |
| | GCCGCGCUCGGUGGUCUGAGCUCAGUGACCCCCUGGUGCUGGUGAUGACA |
| | GGAGCCUACCCAAAACCCACCCUCUCAGCCCAGCCCAGCCCUGUGGUGACC |
| | UCAGGAGGAAGGGUGACCCUCCAGUGUGAGUCACAGGUGGCAUUUGGCGG |
| | CUUCAUUCUGUGUAAGGAAGGAGAAGAUGAACACCCACAAUGCCUGAACU |
| | CCCAGCCCCAUGCCCGUGGGUCGUCCCGCGCCAUCUUCUCCGUGGGCCCCG |
| | UGAGCCCGAAUCGCAGGUGGUCGCACAGGUGCUAUGGUUAUGACUUGAAC |
| | UCUCCCUAUGUGUGGUCUUCACCCAGUGAUCUCCUGGAGCUCCUGGUCCC |
| | AGGUGUUUCUAAGAAGCCAUCACUCUCAGUGCAGCCGGGUCCUGUCAUGG |
| | CCCCUGGGGAAAGCCUGACCCUCCAGUGUGUCUCUGAUGUCGGCUAUGAC |
| | AGAUUUGUUCUGUACAAGGAGGGGGAACGUGACCUUCGCCAGCUCCCUGG |
| | CCGGCAGCCCCAGGCUGGGCUCUCCCAGGCCAACUUCACCCUGGGCCCUGU |
| | GAGCCGCUCCUACGGGGGCCAGUACAGAUGCUACGGUGCACACAACCUCU |
| | CCUCUGAGUGCUCGGCCCCCAGCGACCCCCUGGACAUCCUGAUCACAGGAC |
| | AGAUCCGUGGCACACCCUUCAUCUCAGUGCAGCCAGGCCCCACAGUGGCCU |
| | CAGGAGAGAACGUGACCCUGCUGUGUCAGUCAUGGCGGCAGUUCCACACU |
| | UUCCUUCUGACCAAGGCGGGAGCAGCUGAUGCCCCACUCCGUCUAAGAUC |
| | AAUACACGAAUAUCCUAAGUACCAGGCUGAAUUCCCCAUGAGUCCUGUGA |
| | CCUCAGCCCACGCGGGGACCUACAGGUGCUACGGCUCACUCAACUCCGACC |
| | CCUACCUGCUGUCUCACCCCAGUGAGCCCCUGGAGCUCGUGGUCUCAGGAC |
| | CCUCCAUGGGUUCCAGCCCCCCACCCACCGGUCCCAUCUCCACACCUGGCC |
| | CUGAGGACCAGCCCCUCACCCCCACUGGGUCGGAUCCCCAAAGUGGUCUGG |
| | GAAGGCACCUGGGGGUUGUGAUCGGCAUCUUGGUGGCCGUCGUCCUACUG |
| | CUCCUCCUCCUCCUCCUCCUCUUCCUCAUCCUCCGACAUCGACGUCAGGGC |
| | AAACACUGGACAUCGACCCAGAGAAAGGCUGAUUUCCAACAUCCUGCAGG |
| | GGCUGUGGGGCCAGAGCCCACAGACAGAGGCCUGCAGUGGAGGUCCAGCC |
| | CAGCUGCCGACGCCCAGGAAGAAAACCUCUAUGCUGCCGUGAAGGACACA |
| | CAGCCUGAAGAUGGGGUGGAGAUGGACACUCGGGCUGCUGCAUCUGAAGC |
| | CCCCCAGGAUGUGACCUACGCCCAGCUGCACAGCUUGACCCUCAGACGGAA |
| | GGCAACUGAGCCUCCUCCAUCCCAGGAAAGGGAACCUCCAGCUGAGCCCA |
| | GCAUCUACGCCACCCUGGCCAUCCACUAGCCCGGAGGGUACGCAGACUCCA |
| | CACUCAGUAGAAGGAGACUCAGGACUGCUGAAGGCACGGGAGCUGCCCCC |
| | AGUGGACACCAAUGAACCCCAGUCAGCCUGGACCCCUAACAAAGACCAUG |
| | AGGAGAUGCUGGGAACUUUGGGACUCACUUGAUUCUGCAGUCGAAAUAAC |
| | UAAUAUCCCUACAUUUUUUAAUUAAAGCAACAGACUUCUCAAUAAUCAAU |
| | GAGUUAACCGAGAAAACUAAAAUCAGAAGUAAGAAUGUGCUUUAAACUGA |
| | AUCACAAUAUAAAUAUUACACAUCACACAAUGAAAUUGAAAAAGUACAAA |
| | CCACAAAUGAAAAAAGUAGAAACGAAAAAAAAAAACUAGGAAAUGAAUG |
| | ACGUUGGCUUUCGUAUAAGGAAUUUAGAAAAAGAAUAACCAAUUAUUCCA |
| | AAUGAAGGUGUAAGAAAGGGAAUAAGAAGAAGAAGAGUUGCUCAUGAGG |
| | AAAAACCAAAACUUGAAAAUUCAACAAAGCCAAUGAAGCUCAUUCUUGAA |
| | AAUAUUAAUUACAGUCAUAAAUCCUAACUACAUUGAGCAAGAGAAAGAAA |
| | GAGCAGGCACGCAUUUCCAUAUGGGAGUGAGCCAGCAGACAGCCCAGCAG |
| | AUCCUACACACAUUUUCACAAACUAACCCCAGAACAGGCUGCAAACCUAU |
| | ACCAAUAUACUAGAAAAUGCAGAUUAAAUGGAUGAAAUAUUCAAAACUGG |
| | AGUUUACAUAAUGAACGUAAGAGUAAUCAGAGAAUCUGACUCAUUUUAAA |
| | UGUGUGUGUAUGUGUGUGUAUAUAUAUGUGUGUGUGUGUGUGUGUGUGU |
| | GUGUGUGAAAAACAUUGACUGUAAUAAAAAUGUUCCCAUCGUAUCAACUC |
| | CA (SEQ ID NO: 300) |
| LILRB4 | ACUGAGGACUCAUCCAUCUGCACAGCUGGGGCCCCUGGGAGGAGACGCCA |
| NM_ | UGAUCCCCACCUUCACGGCUCUGCUCUGCCUCGGGCUGAGUCUGGGCCCCA |
| 001278426 | GGACCCACAUGCAGGCAGGGCCCCUCCCCAAACCCACCCUCUGGGCUGAGC |
| 11006 | CAGGCUCUGUGAUCAGCUGGGGGAACUCUGUGACCAUCUGGUGUCAGGGG |
| | ACCCUGGAGGCUCGGGAGUACCGUCUGGAUAAAGAGGAAAGCCCAGCACC |
| | CUGGGACAGACAGAACCCACUGGAGCCCAAGAACAAGGCCAGAUUCUCCA |
| | UCCCAUCCAUGACAGAGGACUAUGCAGGGAGAUACCGCUGUUACUAUCGC |
| | AGCCCUGUAGGCUGGUCACAGCCCAGUGACCCCCUGGAGCUGGUGAUGAC |
| | AGGAGCCUACAGUAAACCCACCCUUUCAGCCCUGCCGAGUCCUCUUGUGA |
| | CCUCAGGAAAGAGCGUGACCCUGCUGUGUCAGUCACGGAGCCCAAUGGAC |
| | ACUUUUCUUCUGAUCAAGGAGCGGGCAGCCCAUCCCCUACUGCAUCUGAG |
| | AUCAGAGCACGGAGCUCAGCAGCACCAGGCUGAAUUCCCCAUGAGUCCUG |
| | UGACCUCAGUGCACGGGGGGACCUACAGGUGCUUCAGCUCACACGGCUUC |
| | UCCCACUACCUGCUGUCACACCCCAGUGACCCCCUGGAGCUCAUAGUCUCA |
| | GGAUCCUUGGAGGGUCCCAGGCCCUCACCCACAAGGUCCGUCUCAACAGC |
| | UGCAGGCCCUGAGGACCAGCCCCUCAUGCCUACAGGGUCAGUCCCCCACAG |
| | UGGUCUGAGAAGGCACUGGGAGGUACUGAUCGGGGUCUUGGUGGUCUCCA |
| | UCCUGCUUCUCUCCCUCCUCCUCUUCCUCCUCCUCCAACACUGGCGUCAGG |
| | GAAAACACAGGACAUUGGCCCAGAGACAGGCUGAUUUCCAACGUCCUCCA |
| | GGGGCUGCCGAGCCAGAGCCCAAGGACGGGGGCCUACAGAGGAGGUCCAG |
| | CCCAGCUGCUGACGUCCAGGGAGAAAACUUCUGUGCUGCCGUGAAGAACA |
| | CACAGCCUGAGGACGGGGUGGAAAUGGACACUCGGCAGAGCCCACACGAU |
| | GAAGACCCCCAGGCAGUGACGUAUGCCAAGGUGAAACACUCCAGACCUAG |
| | GAGAGAAAUGGCCUCUCCUCCCUCCCCACUGUCUGGGGAAUUCCUGGACA |
| | CAAAGGACAGACAGGCAGAAGAGGACAGACAGAUGGACACUGAGGCUGCU |
| | GCAUCUGAAGCCCCCCAGGAUGUGACCUACGCCCGGCUGCACAGCUUUACC |
| | CUCAGACAGAAGGCAACUGAGCCUCCUCCAUCCCAGGAAGGGGCCUCUCC |
| | AGCUGAGCCCAGUGUCUAUGCCACUCUGGCCAUCCACUAAUCCAGGGGGG |
| | ACCCAGACCCCACAAGCCAUGGAGACUCAGGACCCCAGAAGGCAUGGAAG |
| | CUGCCUCCAGUAGACAUCACUGAACCCCAGCCAGCCCAGACCCCUGACACA |
| | GACCACUAGAAGAUUCCGGGAACGUUGGGAGUCACCUGAUUCUGCAAAGA |
| | UAAAUAAUAUCCCUGCAUUAUCAAAAUAAAGUAGCAGACCUCUCAAUUCA |
| | CAAUGAGUUAACUGAUAAAACAAAACAGAAGUCAGACAAUGUUUUAAAUU |
| | GAAUGAUCAUGUAAAUAUUACACAUCAAACCAAUGACAUGGGAAAAUGGG |
| | AGCUUCUAAUGAGGACAAACAAAAAAUAGAGAAAAAUUAAUAAAGUCAA |
| | AAUGUUUAUUCUUGAAAACAUUAAUGAUACAUGAAUCUUGGCCACAAUGA |
| | GAAAAAUAAAAAUGAAAAAAGAGCAGGCAUCCAUUUCCAUACAGGAACAA |
| | AAUAGGAGGCAGCACUACAGACCCUACACACAGCUUUACAGAGGUGAAAG |
| | AAAACUGUCAGCAAUUCUAUGCUGACAUAACAGAAAAUGUAGAUGAGAUA |
| | GAUGAAAUACGAAAAAUUACAGUUUACUUAAUGAACAUAAGGAUAAAUA |
| | GAAAAACUGAAUCAUCAUACAUAAACAUAUAUAAAAUGCAUUGAUCCUGU |
| | AAUCAAAAAUGUUCCCACAAAGUAAAUGCCACUUCAGCAA (SEQ ID NO: |
| | 301) |
| HAVCR2 | AUUUGGAGAGUUAAAACUGUGCCUAACAGAGGUGUCCUCUGACUUUUCUU |
| NM_032782 | CUGCAAGCUCCAUGUUUUCACAUCUUCCCUUUGACUGUGUCCUGCUGCUG |
| 84868 | CUGCUGCUACUACUUACAAGGUCCUCAGAAGUGGAAUACAGAGCGGAGGU |
| | CGGUCAGAAUGCCUAUCUGCCCUGCUUCUACACCCCAGCCGCCCCAGGGAA |
| | CCUCGUGCCCGUCUGCUGGGGCAAAGGAGCCUGUCCUGUGUUUGAAUGUG |
| | GCAACGUGGUGCUCAGGACUGAUGAAAGGGAUGUGAAUUAUUGGACAUCC |
| | AGAUACUGGCUAAAUGGGGAUUUCCGCAAAGGAGAUGUGUCCCUGACCAU |
| | AGAGAAUGUGACUCUAGCAGACAGUGGGAUCUACUGCUGCCGGAUCCAAA |
| | UCCCAGGCAUAAUGAAUGAUGAAAAAUUUAACCUGAAGUUGGUCAUCAAA |
| | CCAGCCAAGGUCACCCCUGCACCGACUCGGCAGAGAGACUUCACUGCAGCC |
| | UUUCCAAGGAUGCUUACCACCAGGGGACAUGGCCCAGCAGAGACACAGAC |
| | ACUGGGGAGCCUCCCUGAUAUAAAUCUAACACAAAUAUCCACAUUGGCCA |
| | AUGAGUUACGGGACUCUAGAUUGGCCAAUGACUUACGGGACUCUGGAGCA |
| | ACCAUCAGAAUAGGCAUCUACAUCGGAGCAGGGAUCUGUGCUGGGCUGGC |
| | UCUGGCUCUUAUCUUCGGCGCUUUAAUUUUCAAAUGGUAUUCUCAUAGCA |
| | AAGAGAAGAUACAGAAUUUAAGCCUCAUCUCUUUGGCCAACCUCCCUCCC |
| | UCAGGAUUGGCAAAUGCAGUAGCAGAGGGAAUUCGCUCAGAAGAAAACAU |
| | CUAUACCAUUGAAGAGAACGUAUAUGAAGUGGAGGAGCCCAAUGAGUAUU |
| | AUUGCUAUGUCAGCAGCAGGCAGCAACCCUCACAACCUUUGGGUUGUCGC |
| | UUUGCAAUGCCAUAGAUCCAACCACCUUAUUUUUGAGCUUGGUGUUUUGU |
| | CUUUUUCAGAAACUAUGAGCUGUGUCACCUGACUGGUUUUGGAGGUUCUG |
| | UCCACUGCUAUGGAGCAGAGUUUUCCCAUUUUCAGAAGAUAAUGACUCAC |
| | AUGGGAAUUGAACUGGGACCUGCACUGAACUUAAACAGGCAUGUCAUUGC |
| | CUCUGUAUUUAAGCCAACAGAGUUACCCAACCCAGAGACUGUUAAUCAUG |
| | GAUGUUAGAGCUCAAACGGGCUUUUAUAUACACUAGGAAUUCUUGACGUG |
| | GGGUCUCUGGAGCUCCAGGAAAUUCGGGCACAUCAUAUGUCCAUGAAACU |
| | UCAGAUAAACUAGGGAAAACUGGGUGCUGAGGUGAAAGCAUAACUUUUUU |
| | GGCACAGAAAGUCUAAAGGGGCCACUGAUUUUCAAAGAGAUCUGUGAUCC |
| | CUUUUUGUUUUUUGUUUUUGAGAUGGAGUCUUGCUCUGUUGCCCAGGCUG |
| | GAGUGCAAUGGCACAAUCUCGGCUCACUGCAAGCUCCGCCUCCUGGGUUC |
| | AAGCGAUUCUCCUGCCUCAGCCUCCUGAGUGGCUGGGAUUACAGGCAUGC |
| | ACCACCAUGCCCAGCUAAUUUGUUGUAUUUUUAGUAGAGACAGGGUUUCA |
| | CCAUGUUGGCCAGUGUGGUCUCAAACUCCUGACCUCAUGAUUUGCCUGCC |
| | UCGGCCUCCCAAAGCACUGGGAUUACAGGCGUGAGCCACCACAUCCAGCC |
| | AGUGAUCCUUAAAAGAUUAAGAGAUGACUGGACCAGGUCUACCUUGAUCU |
| | UGAAGAUUCCCUUGGAAUGUUGAGAUUUAGGCUUAUUUGAGCACUGCCUG |
| | CCCAACUGUCAGUGCCAGUGCAUAGCCCUUCUUUUGUCUCCCUUAUGAAG |
| | ACUGCCCUGCAGGGCUGAGAUGUGGCAGGAGCUCCCAGGGAAAAACGAAG |
| | UGCAUUUGAUUGGUGUGUAUUGGCCAAGUUUUGCUUGUUGUGUGCUUGA |
| | AAGAAAAUAUCUCUGACCAACUUCUGUAUUCGUGGACCAAACUGAAGCUA |
| | UAUUUUUCACAGAAGAAGAAGCAGUGACGGGGACACAAAUUCUGUUGCCU |
| | GGUGGAAAGAAGGCAAAGGCCUUCAGCAAUCUAUAUUACCAGCGCUGGAU |
| | CCUUUGACAGAGAGUGGUCCCUAAACUUAAAUUUCAAGACGGUAUAGGCU |
| | UGAUCUGUCUUGCUUAUUGUUGCCCCCUGCGCCUAGCACAAUUCUGACAC |
| | ACAAUUGGAACUUACUAAAAAUUUUUUUUUACUGUU (SEQ ID NO: 302) |
| SIGLEC10 | ACUCACAGCACGACCAGAGAACAGGCCUGUCUCAGGCAGGCCCUGCGCCUC |
| NM_ | CUAUGCGGAGAUGCUACUGCCACUGCUGCUGUCCUCGCUGCUGGGCGGGU |
| 001171156 | CCCAGGCUAUGGAUGGGAGAUUCUGGAUACGAGUGCAGGAGUCAGUGAUG |
| 89790 | GUGCCGGAGGGCCUGUGCAUCUCUGUGCCCUGCUCUUUCUCCUACCCCCGA |
| | CAGGACUGGACAGGGUCUACCCCAGCUUAUGGCUACUGGUUCAAAGCAGU |
| | GACUGAGACAACCAAGGGUGCUCCUGUGGCCACAAACCACCAGAGUCGAG |
| | AGGUGGAAAUGAGCACCCGGGGCCGAUUCCAGCUCACUGGGGAUCCCGCC |
| | AAGGGGAACUGCUCCUUGGUGAUCAGAGACGCGCAGAUGCAGGAUGAGUC |
| | ACAGUACUUCUUUCGGGUGGAGAGAGGAAGCUAUGUGAGAUAUAAUUUCA |
| | UGAACGAUGGGUUCUUUCUAAAAGUAACAGUGCUCAGCUUCACGCCCAGA |
| | CCCCAGGACCACAACACCGACCUCACCUGCCAUGUGGACUUCUCCAGAAAG |
| | GGUGUGAGCGCACAGAGGACCGUCCGACUCCGUGUGGCCUAUGCCCCCAG |
| | AGACCUUGUUAUCAGCAUUUCACGUGACAACACGCCAGCCCUGGAGCCCC |
| | AGCCCCAGGGAAAUGUCCCAUACCUGGAAGCCCAAAAAGGCCAGUUCCUG |
| | CGGCUCCUCUGUGCUGCUGACAGCCAGCCCCCUGCCACACUGAGCUGGGUC |
| | CUGCAGAACAGAGUCCUCUCCUCGUCCCAUCCCUGGGGCCCUAGACCCCUG |
| | GGGCUGGAGCUGCCCGGGGUGAAGGCUGGGGAUUCAGGGCGCUACACCUG |
| | CCGAGCGGAGAACAGGCUUGGCUCCCAGCAGCGAGCCCUGGACCUCUCUG |
| | UGCAGUAUCCUCCAGAGAACCUGAGAGUGAUGGUUUCCCAAGCAAACAGG |
| | ACAGUCCUGGAAAACCUUGGGAACGGCACGUCUCUCCCAGUACUGGAGGG |
| | CCAAAGCCUGUGCCUGGUCUGUGUCACACACAGCAGCCCCCCAGCCAGGCU |
| | GAGCUGGACCCAGAGGGGACAGGUUCUGAGCCCCUCCCAGCCCUCAGACCC |
| | CGGGGUCCUGGAGCUGCCUCGGGUUCAAGUGGAGCACGAAGGAGAGUUCA |
| | CCUGCCACGCUCGGCACCCACUGGGCUCCCAGCACGUCUCUCUCAGCCUCU |
| | CCGUGCACUACUCCCCGAAGCUGCUGGGCCCCUCCUGCUCCUGGGAGGCUG |
| | AGGGUCUGCACUGCAGCUGCUCCUCCCAGGCCAGCCCGGCCCCCUCUCUGC |
| | GCUGGUGGCUUGGGGAGGAGCUGCUGGAGGGGAACAGCAGCCAGGACUCC |
| | UUCGAGGUCACCCCCAGCUCAGCCGGGCCCUGGGCCAACAGCUCCCUGAGC |
| | CUCCAUGGAGGGCUCAGCUCCGGCCUCAGGCUCCGCUGUGAGGCCUGGAA |
| | CGUCCAUGGGGCCCAGAGUGGAUCCAUCCUGCAGCUGCCAGAUAAGAAGG |
| | GACUCAUCUCAACGGCAUUCUCCAACGGAGCGUUUCUGGGAAUCGGCAUC |
| | ACGGCUCUUCUUUUCCUCUGCCUGGCCCUGAUCAUCAUGAAGAUUCUACC |
| | GAAGAGACGGACUCAGACAGAAACCCCGAGGCCCAGGUUCUCCCGGCACA |
| | GCACGAUCCUGGAUUACAUCAAUGUGGUCCCGACGGCUGGCCCCCUGGCU |
| | CAGAAGCGGAAUCAGAAAGCCACACCAAACAGUCCUCGGACCCCUCUUCC |
| | ACCAGGUGCUCCCUCCCCAGAAUCAAAGAAGAACCAGAAAAAGCAGUAUC |
| | AGUUGCCCAGUUUCCCAGAACCCAAAUCAUCCACUCAAGCCCCAGAAUCCC |
| | AGGAGAGCCAAGAGGAGCUCCAUUAUGCCACGCUCAACUUCCCAGGCGUC |
| | AGACCCAGGCCUGAGGCCCGGAUGCCCAAGGGCACCCAGGCGGAUUAUGC |
| | AGAAGUCAAGUUCCAAUGAGGGUCUCUUAGGCUUUAGGACUGGGACUUCG |
| | GCUAGGGAGGAAGGUAGAGUAAGAGGUUGAAGAUAACAGAGUGCAAAGU |
| | UUCCUUCUCUCCCUCUCUCUCUCUCUUUCUCUCUCUCUCUCUCUUUCUCUC |
| | UCUUUUAAAAAAACAUCUGGCCAGGGCACAGUGGCUCACGCCUGUAAUCC |
| | CAGCACUUUGGGAGGUUGAGGUGGGCAGAUCGCCUGAGGUCGGGAGUUCG |
| | AGACCAGCCUGGCCAACUUGGUGAAACCCCGUCUCUACUAAAAAUACAAA |
| | AAUUAGCUGGGCAUGGUGGCAGGCGCCUGUAAUCCUACCUACUUGGGAAG |
| | CUGAGGCAGGAGAAUCACUUGAACCUGGGAGACGGAGGUUGCAGUGAGCC |
| | AAGAUCACACCAUUGCACGCCAGCCUGGGCAACAAAGCGAGACUCCAUCU |
| | CAAAAAAAAAAUCCUCCAAAUGGGUUGGGUGUCUGUAAUCCCAGCACUUU |
| | GGGAGGCUAAGGUGGGUGGAUUGCUUGAGCCCAGGAGUUCGAGACCAGCC |
| | UGGGCAACAUGGUGAAACCCCAUCUCUACAAAAAAUACAAAACAUAGCUG |
| | GGCUUGGUGGUGUGUGCCUGUAGUCCCAGCUGUCAGACAUUUAAACCAGA |
| | GCAACUCCAUCUGGAAUAGGAGCUGAAUAAAAUGAGGCUGAGACCUACUG |
| | GGCUGCAUUCUCAGACAGUGGAGGCAUUCUAAGUCACAGGAUGAGACAGG |
| | AGGUCCGUACAAGAUACAGGUCAUAAAGACUUUGCUGAUAAAACAGAUUG |
| | CAGUAAAGAAGCCAACCAAAUCCCACCAAAACCAAGUUGGCCACGAGAGU |
| | GACCUCUGGUCGUCCUCACUGCUACACUCCUGACAGCACCAUGACAGUUU |
| | ACAAAUGCCAUGGCAACAUCAGGAAGUUACCCGAUAUGUCCCAAAAGGGG |
| | GAGGAAUGAAUAAUCCACCCCUUGUUUAGCAAAUAAGCAAGAAAUAACCA |
| | UAAAAGUGGGCAACCAGCAGCUCUAGGCGCUGCUCUUGUCUAUGGAGUAG |
| | CCAUUCUUUUGUUCCUUUACUUUCUUAAUAAACUUGCUUUCACCUUAC |
| | (SEQ ID NO: 303) |
| TREM2 | ACUCUGCUUCUGCCCUUGGCUGGGGAAGGGUGGCAUGGAGCCUCUCCGGC |
| NM_ | UGCUCAUCUUACUCUUUGUCACAGAGCUGUCCGGAGCCCACAACACCACA |
| 001271821 | GUGUUCCAGGGCGUGGCGGGCCAGUCCCUGCAGGUGUCUUGCCCCUAUGA |
| 54209 | CUCCAUGAAGCACUGGGGGAGGCGCAAGGCCUGGUGCCGCCAGCUGGGAG |
| | AGAAGGGCCCAUGCCAGCGUGUGGUCAGCACGCACAACUUGUGGCUGCUG |
| | UCCUUCCUGAGGAGGUGGAAUGGGAGCACAGCCAUCACAGACGAUACCCU |
| | GGGUGGCACUCUCACCAUUACGCUGCGGAAUCUACAACCCCAUGAUGCGG |
| | GUCUCUACCAGUGCCAGAGCCUCCAUGGCAGUGAGGCUGACACCCUCAGG |
| | AAGGUCCUGGUGGAGGUGCUGGCAGACCCCCUGGAUCACCGGGAUGCUGG |
| | AGAUCUCUGGUUCCCCGGGGAGUCUGAGAGCUUCGAGGAUGCCCAUGUGG |
| | AGCACAGCAUCUCCAGGGCUGAGAGACACGUGAAGGAAGAUGAUGGGAGG |
| | AAAAGCCCAGGAGAAGUCCCACCAGGGACCAGCCCAGCCUGCAUACUUGC |
| | CACUUGGCCACCAGGACUCCUUGUUCUGCUCUGGCAAGAGACUACUCUGC |
| | CUGAACACUGCUUCUCCUGGACCCUGGAAGCAGGGACUGGUUGAGGGAGU |
| | GGGGAGGUGGUAAGAACACCUGACAACUUCUGAAUAUUGGACAUUUUAAA |
| | CACUUACAAAUAAAUCCAAGACUGUCAUAUUUAGCUGGA (SEQ ID NO: |
| | 304) |
| STAT3 | GUCGCAGCCGAGGGAACAAGCCCCAACCGGAUCCUGGACAGGCACCCCGGC |
| NM_ | UUGGCGCUGUCUCUCCCCCUCGGCUCGGAGAGGCCCUUCGGCCUGAGGGA |
| 001369512 | GCCUCGCCGCCCGUCCCCGGCACACGCGCAGCCCCGGCCUCUCGGCCUCUG |
| 6774 | CCGGAGAAACAGGAUGGCCCAAUGGAAUCAGCUACAGCAGCUUGACACAC |
| | GGUACCUGGAGCAGCUCCAUCAGCUCUACAGUGACAGCUUCCCAAUGGAG |
| | CUGCGGCAGUUUCUGGCCCCUUGGAUUGAGAGUCAAGAUUGGGCAUAUGC |
| | GGCCAGCAAAGAAUCACAUGCCACUUUGGUGUUUCAUAAUCUCCUGGGAG |
| | AGAUUGACCAGCAGUAUAGCCGCUUCCUGCAAGAGUCGAAUGUUCUCUAU |
| | CAGCACAAUCUACGAAGAAUCAAGCAGUUUCUUCAGAGCAGGUAUCUUGA |
| | GAAGCCAAUGGAGAUUGCCCGGAUUGUGGCCCGGUGCCUGUGGGAAGAAU |
| | CACGCCUUCUACAGACUGCAGCCACUGCGGCCCAGCAAGGGGGCCAGGCCA |
| | ACCACCCCACAGCAGCCGUGGUGACGGAGAAGCAGCAGAUGCUGGAGCAG |
| | CACCUUCAGGAUGUCCGGAAGAGAGUGCAGGAUCUAGAACAGAAAAUGAA |
| | AGUGGUAGAGAAUCUCCAGGAUGACUUUGAUUUCAACUAUAAAACCCUCA |
| | AGAGUCAAGGAGACAUGCAAGAUCUGAAUGGAAACAACCAGUCAGUGACC |
| | AGGCAGAAGAUGCAGCAGCUGGAACAGAUGCUCACUGCGCUGGACCAGAU |
| | GCGGAGAAGCAUCGUGAGUGAGCUGGCGGGGCUUUUGUCAGCGAUGGAGU |
| | ACGUGCAGAAAACUCUCACGGACGAGGAGCUGGCUGACUGGAAGAGGCGG |
| | CAACAGAUUGCCUGCAUUGGAGGCCCGCCCAACAUCUGCCUAGAUCGGCU |
| | AGAAAACUGGAUAACGUCAUUAGCAGAAUCUCAACUUCAGACCCGUCAAC |
| | AAAUUAAGAAACUGGAGGAGUUGCAGCAAAAAGUUUCCUACAAAGGGGAC |
| | CCCAUUGUACAGCACCGGCCGAUGCUGGAGGAGAGAAUCGUGGAGCUGUU |
| | UAGAAACUUAAUGAAAAGUGCCUUUGUGGUGGAGCGGCAGCCCUGCAUGC |
| | CCAUGCAUCCUGACCGGCCCCUCGUCAUCAAGACCGGCGUCCAGUUCACUA |
| | CUAAAGUCAGGUUGCUGGUCAAAUUCCCUGAGUUGAAUUAUCAGCUUAAA |
| | AUUAAAGUGUGCAUUGACAAAGACUCUGGGGACGUUGCAGCUCUCAGAGG |
| | AUCCCGGAAAUUUAACAUUCUGGGCACAAACACAAAAGUGAUGAACAUGG |
| | AAGAAUCCAACAACGGCAGCCUCUCUGCAGAAUUCAAACACUUGACCCUG |
| | AGGGAGCAGAGAUGUGGGAAUGGGGGCCGAGCCAAUUGUGAUGCUUCCCU |
| | GAUUGUGACUGAGGAGCUGCACCUGAUCACCUUUGAGACCGAGGUGUAUC |
| | ACCAAGGCCUCAAGAUUGACCUAGAGACCCACUCCUUGCCAGUUGUGGUG |
| | AUCUCCAACAUCUGUCAGAUGCCAAAUGCCUGGGCGUCCAUCCUGUGGUA |
| | CAACAUGCUGACCAACAAUCCCAAGAAUGUAAACUUUUUUACCAAGCCCC |
| | CAAUUGGAACCUGGGAUCAAGUGGCCGAGGUCCUGAGCUGGCAGUUCUCC |
| | UCCACCACCAAGCGAGGACUGAGCAUCGAGCAGCUGACUACACUGGCAGA |
| | GAAACUCUUGGGACCUGGUGUGAAUUAUUCAGGGUGUCAGAUCACAUGGG |
| | CUAAAUUUUGCAAAGAAAACAUGGCUGGCAAGGGCUUCUCCUUCUGGGUC |
| | UGGCUGGACAAUAUCAUUGACCUUGUGAAAAAGUACAUCCUGGCCCUUUG |
| | GAACGAAGGGUACAUCAUGGGCUUUAUCAGUAAGGAGCGGGAGCGGGCCA |
| | UCUUGAGCACUAAGCCUCCAGGCACCUUCCUGCUAAGAUUCAGUGAAAGC |
| | AGCAAAGAAGGAGGCGUCACUUUCACUUGGGUGGAGAAGGACAUCAGCGG |
| | UAAGACCCAGAUCCAGUCCGUGGAACCAUACACAAAGCAGCAGCUGAACA |
| | ACAUGUCAUUUGCUGAAAUCAUCAUGGGCUAUAAGAUCAUGGAUGCUACC |
| | AAUAUCCUGGUGUCUCCACUGGUCUAUCUCUAUCCUGACAUUCCCAAGGA |
| | GGAGGCAUUCGGAAAGUAUUGUCGGCCAGAGAGCCAGGAGCAUCCUGAAG |
| | CUGACCCAGGUAGCGCUGCCCCAUACCUGAAGACCAAGUUUAUCUGUGUG |
| | ACACCAACGACCUGCAGCAAUACCAUUGACCUGCCGAUGUCCCCCCGCACU |
| | UUAGAUUCAUUGAUGCAGUUUGGAAAUAAUGGUGAAGGUGCUGAACCCUC |
| | AGCAGGAGGGCAGUUUGAGUCCCUCACCUUUGACAUGGAGUUGACCUCGG |
| | AGUGCGCUACCUCCCCCAUGUGAGGAGCUGAGAACGGAAGCUGCAGAAAG |
| | AUACGACUGAGGCGCCUACCUGCAUUCUGCCACCCCUCACACAGCCAAACC |
| | CCAGAUCAUCUGAAACUACUAACUUUGUGGUUCCAGAUUUUUUUUAAUCU |
| | CCUACUUCUGCUAUCUUUGAGCAAUCUGGGCACUUUUAAAAAUAGAGAAA |
| | UGAGUGAAUGUGGGUGAUCUGCUUUUAUCUAAAUGCAAAUAAGGAUGUG |
| | UUCUCUGAGACCCAUGAUCAGGGGAUGUGGCGGGGGGUGGCUAGAGGGAG |
| | AAAAAGGAAAUGUCUUGUGUUGUUUUGUUCCCCUGCCCUCCUUUCUCAGC |
| | AGCUUUUUGUUAUUGUUGUUGUUGUUCUUAGACAAGUGCCUCCUGGUGCC |
| | UGCGGCAUCCUUCUGCCUGUUUCUGUAAGCAAAUGCCACAGGCCACCUAU |
| | AGCUACAUACUCCUGGCAUUGCACUUUUUAACCUUGCUGACAUCCAAAUA |
| | GAAGAUAGGACUAUCUAAGCCCUAGGUUUCUUUUUAAAUUAAGAAAUAAU |
| | AACAAUUAAAGGGCAAAAAACACUGUAUCAGCAUAGCCUUUCUGUAUUUA |
| | AGAAACUUAAGCAGCCGGGCAUGGUGGCUCACGCCUGUAAUCCCAGCACU |
| | UUGGGAGGCCGAGGCGGAUCAUAAGGUCAGGAGAUCAAGACCAUCCUGGC |
| | UAACACGGUGAAACCCCGUCUCUACUAAAAGUACAAAAAAUUAGCUGGGU |
| | GUGGUGGUGGGCGCCUGUAGUCCCAGCUACUCGGGAGGCUGAGGCAGGAG |
| | AAUCGCUUGAACCUGAGAGGCGGAGGUUGCAGUGAGCCAAAAUUGCACCA |
| | CUGCACACUGCACUCCAUCCUGGGCGACAGUCUGAGACUCUGUCUCAAAA |
| | AAAAAAAAAAAAAAAAGAAACUUCAGUUAACAGCCUCCUUGGUGCUUUAA |
| | GCAUUCAGCUUCCUUCAGGCUGGUAAUUUAUAUAAUCCCUGAAACGGGCU |
| | UCAGGUCAAACCCUUAAGACAUCUGAAGCUGCAACCUGGCCUUUGGUGUU |
| | GAAAUAGGAAGGUUUAAGGAGAAUCUAAGCAUUUUAGACUUUUUUUUAU |
| | AAAUAGACUUAUUUUCCUUUGUAAUGUAUUGGCCUUUUAGUGAGUAAGGC |
| | UGGGCAGAGGGUGCUUACAACCUUGACUCCCUUUCUCCCUGGACUUGAUC |
| | UGCUGUUUCAGAGGCUAGGUUGUUUCUGUGGGUGCCUUAUCAGGGCUGGG |
| | AUACUUCUGAUUCUGGCUUCCUUCCUGCCCCACCCUCCCGACCCCAGUCCC |
| | CCUGAUCCUGCUAGAGGCAUGUCUCCUUGCGUGUCUAAAGGUCCCUCAUC |
| | CUGUUUGUUUUAGGAAUCCUGGUCUCAGGACCUCAUGGAAGAAGAGGGGG |
| | AGAGAGUUACAGGUUGGACAUGAUGCACACUAUGGGGCCCCAGCGACGUG |
| | UCUGGUUGAGCUCAGGGAAUAUGGUUCUUAGCCAGUUUCUUGGUGAUAUC |
| | CAGUGGCACUUGUAAUGGCGUCUUCAUUCAGUUCAUGCAGGGCAAAGGCU |
| | UACUGAUAAACUUGAGUCUGCCCUCGUAUGAGGGUGUAUACCUGGCCUCC |
| | CUCUGAGGCUGGUGACUCCUCCCUGCUGGGGCCCCACAGGUGAGGCAGAA |
| | CAGCUAGAGGGCCUCCCCGCCUGCCCGCCUUGGCUGGCUAGCUCGCCUCUC |
| | CUGUGCGUAUGGGAACACCUAGCACGUGCUGGAUGGGCUGCCUCUGACUC |
| | AGAGGCAUGGCCGGAUUUGGCAACUCAAAACCACCUUGCCUCAGCUGAUC |
| | AGAGUUUCUGUGGAAUUCUGUUUGUUAAAUCAAAUUAGCUGGUCUCUGAA |
| | UUAAGGGGGAGACGACCUUCUCUAAGAUGAACAGGGUUCGCCCCAGUCCU |
| | CCUGCCUGGAGACAGUUGAUGUGUCAUGCAGAGCUCUUACUUCUCCAGCA |
| | ACACUCUUCAGUACAUAAUAAGCUUAACUGAUAAACAGAAUAUUUAGAAA |
| | GGUGAGACUUGGGCUUACCAUUGGGUUUAAAUCAUAGGGACCUAGGGCGA |
| | GGGUUCAGGGCUUCUCUGGAGCAGAUAUUGUCAAGUUCAUGGCCUUAGGU |
| | AGCAUGUAUCUGGUCUUAACUCUGAUUGUAGCAAAAGUUCUGAGAGGAGC |
| | UGAGCCCUGUUGUGGCCCAUUAAAGAACAGGGUCCUCAGGCCCUGCCCGC |
| | UUCCUGUCCACUGCCCCCUCCCCAUCCCCAGCCCAGCCGAGGGAAUCCCGU |
| | GGGUUGCUUACCUACCUAUAAGGUGGUUUAUAAGCUGCUGUCCUGGCCAC |
| | UGCAUUCAAAUUCCAAUGUGUACUUCAUAGUGUAAAAAUUUAUAUUAUUG |
| | UGAGGUUUUUUGUCUUUUUUUUUUUUUUUUUUUUUUGGUAUAUUGCUGU |
| | AUCUACUUUAACUUCCAGAAAUAAACGUUAUAUAGGAACCGUC (SEQ ID |
| | NO: 305) |
| STAT6 | GGGGCAGCCACUGCUUACACUGAAGAGGGAGGACGGGAGAGGAGUGUGUG |
| NM_ | UGUGUGUGUGUGUGUGUGUGUGUAUGUAUGUGUGUGCUUUAUCUUAUUU |
| 001178078 | UUCUUUUUGGUGGUGGUGGUGGAAGGGGGGAGGUGCUAGCAGGGCCAGCC |
| 6778 | UUGAACUCGCUGGACAGAGCUACAGACCUAUGGGGCCUGGAAGUGCCCGC |
| | UGAGAAAGGGAGAAGACAGCAGAGGGGUUGCCGAGGUGAGGGGUUGCCUC |
| | CGAGGCAACCUCCAAGUCCCAGAUCAUGUCUCUGUGGGGUCUGGUCUCCA |
| | AGAUGCCCCCAGAAAAAGUGCAGCGGCUCUAUGUCGACUUUCCCCAACAC |
| | CUGCGGCAUCUUCUGGGUGACUGGCUGGAGAGCCAGCCCUGGGAGUUCCU |
| | GGUCGGCUCCGACGCCUUCUGCUGCAACUUGGCUAGUGCCCUACUUUCAG |
| | ACACUGUCCAGCACCUUCAGGCCUCGGUGGGAGAGCAGGGGGAGGGGAGC |
| | ACCAUCUUGCAACACAUCAGCACCCUUGAGAGCAUAUAUCAGAGGGACCC |
| | CCUGAAGCUGGUGGCCACUUUCAGACAAAUACUUCAAGGAGAGAAAAAAG |
| | CUGUUAUGGAACAGUUCCGCCACUUGCCAAUGCCUUUCCACUGGAAGCAG |
| | GAAGAACUCAAGUUUAAGACAGGCUUGCGGAGGCUGCAGCACCGAGUAGG |
| | GGAGAUCCACCUUCUCCGAGAAGCCCUGCAGAAGGGGGCUGAGGCUGGCC |
| | AAGUGUCUCUGCACAGCUUGAUAGAAACUCCUGCUAAUGGGACUGGGCCA |
| | AGUGAGGCCCUGGCCAUGCUACUGCAGGAGACCACUGGAGAGCUAGAGGC |
| | AGCCAAAGCCCUAGUGCUGAAGAGGAUCCAGAUUUGGAAACGGCAGCAGC |
| | AGCUGGCAGGGAAUGGCGCACCGUUUGAGGAGAGCCUGGCCCCACUCCAG |
| | GAGAGGUGUGAAAGCCUGGUGGACAUUUAUUCCCAGCUACAGCAGGAGGU |
| | AGGGGCGGCUGGUGGGGAGCUUGAGCCCAAGACCCGGGCAUCGCUGACUG |
| | GCCGGCUGGAUGAAGUCCUGAGAACCCUCGUCACCAGUUGCUUCCUGGUG |
| | GAGAAGCAGCCCCCCCAGGUACUGAAGACUCAGACCAAGUUCCAGGCUGG |
| | AGUUCGAUUCCUGUUGGGCUUGAGGUUCCUGGGGGCCCCAGCCAAGCCUC |
| | CGCUGGUCAGGGCCGACAUGGUGACAGAGAAGCAGGCGCGGGAGCUGAGU |
| | GUGCCUCAGGGUCCUGGGGCUGGAGCAGAAAGCACUGGAGAAAUCAUCAA |
| | CAACACUGUGCCCUUGGAGAACAGCAUUCCUGGGAACUGCUGCUCUGCCC |
| | UGUUCAAGAACCUGCUUCUCAAGAAGAUCAAGCGGUGUGAGCGGAAGGGC |
| | ACUGAGUCUGUCACAGAGGAGAAGUGCGCUGUGCUCUUCUCUGCCAGCUU |
| | CACACUUGGCCCCGGCAAACUCCCCAUCCAGCUCCAGGCCCUGUCUCUGCC |
| | CCUGGUGGUCAUCGUCCAUGGCAACCAAGACAACAAUGCCAAAGCCACUA |
| | UCCUGUGGGACAAUGCCUUCUCUGAGAUGGACCGCGUGCCCUUUGUGGUG |
| | GCUGAGCGGGUGCCCUGGGAGAAGAUGUGUGAAACUCUGAACCUGAAGUU |
| | CAUGGCUGAGGUGGGGACCAACCGGGGGCUGCUCCCAGAGCACUUCCUCU |
| | UCCUGGCCCAGAAGAUCUUCAAUGACAACAGCCUCAGUAUGGAGGCCUUC |
| | CAGCACCGUUCUGUGUCCUGGUCGCAGUUCAACAAGGAGAUCCUGCUGGG |
| | CCGUGGCUUCACCUUUUGGCAGUGGUUUGAUGGUGUCCUGGACCUCACCA |
| | AACGCUGUCUCCGGAGCUACUGGUCUGACCGGCUGAUCAUUGGCUUCAUC |
| | AGCAAACAGUACGUUACUAGCCUUCUUCUCAAUGAGCCCGACGGAACCUU |
| | UCUCCUCCGCUUCAGCGACUCAGAGAUUGGGGGCAUCACCAUUGCCCAUG |
| | UCAUCCGGGGCCAGGAUGGCUCUCCACAGAUAGAGAACAUCCAGCCAUUC |
| | UCUGCCAAAGACCUGUCCAUUCGCUCACUGGGGGACCGAAUCCGGGAUCU |
| | UGCUCAGCUCAAAAAUCUCUAUCCCAAGAAGCCCAAGGAUGAGGCUUUCC |
| | GGAGCCACUACAAGCCUGAACAGAUGGGUAAGGAUGGCAGGGGUUAUGUC |
| | CCAGCUACCAUCAAGAUGACCGUGGAAAGGGACCAACCACUUCCUACCCC |
| | AGAGCUCCAGAUGCCUACCAUGGUGCCUUCUUAUGACCUUGGAAUGGCCC |
| | CUGAUUCCUCCAUGAGCAUGCAGCUUGGCCCAGAUAUGGUGCCCCAGGUG |
| | UACCCACCACACUCUCACUCCAUCCCCCCGUAUCAAGGCCUCUCCCCAGAA |
| | GAAUCAGUCAACGUGUUGUCAGCCUUCCAGGAGCCUCACCUGCAGAUGCC |
| | CCCCAGCCUGGGCCAGAUGAGCCUGCCCUUUGACCAGCCUCACCCCCAGGG |
| | CCUGCUGCCGUGCCAGCCUCAGGAGCAUGCUGUGUCCAGCCCUGACCCCCU |
| | GCUCUGCUCAGAUGUGACCAUGGUGGAAGACAGCUGCCUGAGCCAGCCAG |
| | UGACAGCGUUUCCUCAGGGCACUUGGAUUGGUGAAGACAUAUUCCCUCCU |
| | CUGCUGCCUCCCACUGAACAGGACCUCACUAAGCUUCUCCUGGAGGGGCA |
| | AGGGGAGUCGGGGGGAGGGUCCUUGGGGGCACAGCCCCUCCUGCAGCCCU |
| | CCCACUAUGGGCAAUCUGGGAUCUCAAUGUCCCACAUGGACCUAAGGGCC |
| | AACCCCAGUUGGUGAUCCCAGCUGGAGGGAGAACCCAAAGAGACAGCUCU |
| | UCUACUACCCCCACAGACCUGCUCUGGACACUUGCUCAUGCCCUGCCAAGC |
| | AGCAGAUGGGGAGGGUGCCCUCCUAUCCCCACCUACUCCUGGGUCAGGAG |
| | GAAAAGACUAACAGGAGAAUGCACAGUGGGUGGAGCCAAUCCACUCCUUC |
| | CUUUCUAUCAUUCCCCUGCCCACCUCCUUCCAGCACUGACUGGAAGGGAA |
| | GUUCAGGCUCUGAGACACACCCCAACAUGCCUGCACCUGCAGCGCGCACAC |
| | GCACGCACACACACAUACAGAGCUCUCUGAGGGUGAUGGGGCUGAGCAGG |
| | AGGGGGGCUGGGUAAGAGCACAGGUUAGGGCAUGGAAGGCUUCUCCGCCC |
| | AUUCUGACCCAGGGCCUAGGACGGAUAGGCAGGAACAUACAGACACAUUU |
| | ACACUAGAGGCCAGGGAUAGAGGAUAUUGGGUCUCAGCCCUAGGGGAAUG |
| | GGAAGCAGCUCAAGGGACCCUGGGUGGGAGCAUAGGAGGGGUCUGGACAU |
| | GUGGUUACUAGUACAGGUUUUGCCCUGAUUAAAAAAUCUCCCAAAGCCCC |
| | AAAUUCCUGUUAGCCAGGUGGAGGCUUCUGAUACGUGUAUGAGACUAUGC |
| | AAAAGUACAAGGGCUGAGAUUCUUCGUGUAUAGCUGUGUGAACGUGUAUG |
| | UACCUAGGAUAUGUUAAAUGUAUAGCUGGCACCUUAGUUGCAUGACCACA |
| | UAGAACAUGUGUCUAUCUGCUUUUGCCUACGUGACAACACAAAUUUGGGA |
| | GGGUGAGACACUGCACAGAAGACAGCAGCAAGUGUGCUGGCCUCUCUGAC |
| | AUAUGCUAACCCCCAAAUACUCUGAAUUUGGAGUCUGACUGUGCCCAAGU |
| | GGGUCCAAGUGGCUGUGACAUCUACGUAUGGCUCCACACCUCCAAUGCUG |
| | CCUGGGAGCCAGGGUGAGAGUCUGGGUCCAGGCCUGGCCAUGUGGCCCUC |
| | CAGUGUAUGAGAGGGCCCUGCCUGCUGCAUCUUUUCUGUUGCCCCAUCCA |
| | CCGCCAGCUUCCCUUCACUCCCCUAUCCCAUUCUCCCUCUCAAGGCAGGGG |
| | UCAUAGAUCCUAAGCCAUAAAAUAAAUUUUAUUCCAAAAUAACAAAAUAA |
| | AUAAUCUACUGUACACAAUCUGAAAA (SEQ ID NO: 306) |
| ZFP36 | AGCCUGACUUCAGCGCUCCCACUCUCGGCCGACACCCCUCAUGGCCAACCG |
| NM_ | UUACACCAUGGAUCUGACUGCCAUCUACGAGAGCCUCCUGUCGCUGAGCC |
| 003407 | CUGACGUGCCCGUGCCAUCCGACCAUGGAGGGACUGAGUCCAGCCCAGGC |
| 7538 | UGGGGCUCCUCGGGACCCUGGAGCCUGAGCCCCUCCGACUCCAGCCCGUCU |
| | GGGGUCACCUCCCGCCUGCCUGGCCGCUCCACCAGCCUAGUGGAGGGCCGC |
| | AGCUGUGGCUGGGUGCCCCCACCCCCUGGCUUCGCACCGCUGGCUCCCCGC |
| | CUGGGCCCUGAGCUGUCACCCUCACCCACUUCGCCCACUGCAACCUCCACC |
| | ACCCCCUCGCGCUACAAGACUGAGCUAUGUCGGACCUUCUCAGAGAGUGG |
| | GCGCUGCCGCUACGGGGCCAAGUGCCAGUUUGCCCAUGGCCUGGGCGAGC |
| | UGCGCCAGGCCAAUCGCCACCCCAAAUACAAGACGGAACUCUGUCACAAG |
| | UUCUACCUCCAGGGCCGCUGCCCCUACGGCUCUCGCUGCCACUUCAUCCAC |
| | AACCCUAGCGAAGACCUGGCGGCCCCGGGCCACCCUCCUGUGCUUCGCCAG |
| | AGCAUCAGCUUCUCCGGCCUGCCCUCUGGCCGCCGGACCUCACCACCACCA |
| | CCAGGCCUGGCCGGCCCUUCCCUGUCCUCCAGCUCCUUCUCGCCCUCCAGC |
| | UCCCCACCACCACCUGGGGACCUUCCACUGUCACCCUCUGCCUUCUCUGCU |
| | GCCCCUGGCACCCCCCUGGCUCGAAGAGACCCCACCCCAGUCUGUUGCCCC |
| | UCCUGCCGAAGGGCCACUCCUAUCAGCGUCUGGGGGCCCUUGGGUGGCCU |
| | GGUUCGGACCCCCUCUGUACAGUCCCUGGGAUCCGACCCUGAUGAAUAUG |
| | CCAGCAGCGGCAGCAGCCUGGGGGGCUCUGACUCUCCCGUCUUCGAGGCG |
| | GGAGUUUUUGCACCACCCCAGCCCGUGGCAGCCCCCCGGCGACUCCCCAUC |
| | UUCAAUCGCAUCUCUGUUUCUGAGUGACAAAGUGACUGCCCGGUCAGAUC |
| | AGCUGGAUCUCAGCGGGGAGCCACGUCUCUUGCACUGUGGUCUCUGCAUG |
| | GACCCCAGGGCUGUGGGGACUUGGGGGACAGUAAUCAAGUAAUCCCCUUU |
| | UCCAGAAUGCAUUAACCCACUCCCCUGACCUCACGCUGGGGCAGGUCCCCA |
| | AGUGUGCAAGCUCAGUAUUCAUGAUGGUGGGGGAUGGAGUGUCUUCCGAG |
| | GUUCUUGGGGGAAAAAAAAUUGUAGCAUAUUUAAGGGAGGCAAUGAACCC |
| | UCUCCCCCACCUCUUCCCUGCCCAAAUCUGUCUCCUAGAAUCUUAUGUGCU |
| | GUGAAUAAUAGGCCUUCACUGCCCCUCCAGUUUUUAUAGACCUGAGGUUC |
| | CAGUGUCUCCUGGUAACUGGAACCUCUCCUGAGGGGGAAUCCUGGUGCUC |
| | AAAUUACCCUCCAAAAGCAAGUAGCCAAAGCCGUUGCCAAACCCCACCCA |
| | UAAAUCAAUGGGCCCUUUAUUUAUGACGACUUUAUUUAUUCUAAUAUGAU |
| | UUUAUAGUAUUUAUAUAUAUUGGGUCGUCUGCUUCCCUUGUAUUUUUCUU |
| | CCUUUUUUUGUAAUAUUGAAAACGACGAUAUAAUUAUUAUAAGUAGACU |
| | AUAAUAUAUUUAGUAAUAUAUAUUAUUACCUUAAAAGUCUAUUUUUGUG |
| | UUUUGGGCAUUUUUAAAUAAACAAUCUGAGUGUAA (SEQ ID NO: 307) |
| PIK3CG | GCAACACUUCCUCUGCAUCUGGAUAUGAAGGGAGCCCCAGAAAAGCGGAA |
| NM_ | GAAUUUAGACGCACACUGGGUAGGUUUGAAUUUGUUUUGUUUUCAAAAA |
| 001282426 | UUAAACAAAUGAUCCUUCAGCAUCAUCGCCUCCGCUGCUUUAUCAGGUCG |
| 5294 | CAUAGGGCAUGGAGCUGGAGAACUAUAAACAGCCCGUGGUGCUGAGAGAG |
| | GACAACUGCCGAAGGCGCCGGAGGAUGAAGCCGCGCAGUGCUGCGGCCAG |
| | CCUGUCCUCCAUGGAGCUCAUCCCCAUCGAGUUCGUGCUGCCCACCAGCCA |
| | GCGCAAAUGCAAGAGCCCCGAAACGGCGCUGCUGCACGUGGCCGGCCACG |
| | GCAACGUGGAGCAGAUGAAGGCCCAGGUGUGGCUGCGAGCGCUGGAGACC |
| | AGCGUGGCGGCGGACUUCUACCACCGGCUGGGACCGCAUCACUUCCUCCU |
| | GCUCUAUCAGAAGAAGGGGCAGUGGUACGAGAUCUACGACAAGUACCAGG |
| | UGGUGCAGACUCUGGACUGCCUGCGCUACUGGAAGGCCACGCACCGGAGC |
| | CCGGGCCAGAUCCACCUGGUGCAGCGGCACCCGCCCUCCGAGGAGUCCCAA |
| | GCCUUCCAGCGGCAGCUCACGGCGCUGAUUGGCUAUGACGUCACUGACGU |
| | CAGCAACGUGCACGACGAUGAGCUGGAGUUCACGCGCCGUGGCUUGGUGA |
| | CCCCGCGCAUGGCGGAGGUGGCCAGCCGCGACCCCAAGCUCUACGCCAUGC |
| | ACCCGUGGGUGACGUCCAAGCCCCUCCCGGAGUACCUGUGGAAGAAGAUU |
| | GCCAACAACUGCAUCUUCAUCGUCAUUCACCGCAGCACCACCAGCCAGACC |
| | AUUAAGGUCUCACCCGACGACACCCCCGGCGCCAUCCUGCAGAGCUUCUUC |
| | ACCAAGAUGGCCAAGAAGAAAUCUCUGAUGGAUAUUCCCGAAAGCCAAAG |
| | CGAACAGGAUUUUGUGCUGCGCGUCUGUGGCCGGGAUGAGUACCUGGUGG |
| | GCGAAACGCCCAUCAAAAACUUCCAGUGGGUGAGGCACUGCCUCAAGAAC |
| | GGAGAAGAGAUUCACGUGGUACUGGACACGCCUCCAGACCCGGCCCUAGA |
| | CGAGGUGAGGAAGGAAGAGUGGCCACUGGUGGAUGACUGCACGGGAGUCA |
| | CCGGCUACCAUGAGCAGCUUACCAUCCACGGCAAGGACCACGAGAGUGUG |
| | UUCACCGUGUCCCUGUGGGACUGCGACCGCAAGUUCAGGGUCAAGAUCAG |
| | AGGCAUUGAUAUCCCCGUCCUGCCUCGGAACACCGACCUCACAGUUUUUG |
| | UAGAGGCAAACAUCCAGCAUGGGCAACAAGUCCUUUGCCAAAGGAGAACC |
| | AGCCCCAAACCCUUCACAGAGGAGGUGCUGUGGAAUGUGUGGCUUGAGUU |
| | CAGUAUCAAAAUCAAAGACUUGCCCAAAGGGGCUCUACUGAACCUCCAGA |
| | UCUACUGCGGUAAAGCUCCAGCACUGUCCAGCAAGGCCUCUGCAGAGUCC |
| | CCCAGUUCUGAGUCCAAGGGCAAAGUUCAGCUUCUCUAUUAUGUGAACCU |
| | GCUGCUGAUAGACCACCGUUUCCUCCUGCGCCGUGGAGAAUACGUCCUCC |
| | ACAUGUGGCAGAUAUCUGGGAAGGGAGAAGACCAAGGAAGCUUCAAUGCU |
| | GACAAACUCACGUCUGCAACUAACCCAGACAAGGAGAACUCAAUGUCCAU |
| | CUCCAUUCUUCUGGACAAUUACUGCCACCCGAUAGCCCUGCCUAAGCAUC |
| | AGCCCACCCCUGACCCGGAAGGGGACCGGGUUCGAGCAGAAAUGCCCAAC |
| | CAGCUUCGCAAGCAAUUGGAGGCGAUCAUAGCCACUGAUCCACUUAACCC |
| | UCUCACAGCAGAGGACAAAGAAUUGCUCUGGCAUUUUAGAUACGAAAGCC |
| | UUAAGCACCCAAAAGCAUAUCCUAAGCUAUUUAGUUCAGUGAAAUGGGGA |
| | CAGCAAGAAAUUGUGGCCAAAACAUACCAAUUGUUGGCCAGAAGGGAAGU |
| | CUGGGAUCAAAGUGCUUUGGAUGUUGGGUUAACAAUGCAGCUCCUGGACU |
| | GCAACUUCUCAGAUGAAAAUGUAAGAGCCAUUGCAGUUCAGAAACUGGAG |
| | AGCUUGGAGGACGAUGAUGUUCUGCAUUACCUUCUACAAUUGGUCCAGGC |
| | UGUGAAAUUUGAACCAUACCAUGAUAGCGCCCUUGCCAGAUUUCUGCUGA |
| | AGCGUGGUUUAAGAAACAAAAGAAUUGGUCACUUUUUGUUUUGGUUCUU |
| | GAGAAGUGAGAUAGCCCAGUCCAGACACUAUCAGCAGAGGUUCGCUGUGA |
| | UUCUGGAAGCCUAUCUGAGGGGCUGUGGCACAGCCAUGCUGCACGACUUU |
| | ACCCAACAAGUCCAAGUAAUCGAGAUGUUACAAAAAGUCACCCUUGAUAU |
| | UAAAUCGCUCUCUGCUGAAAAGUAUGACGUCAGUUCCCAAGUUAUUUCAC |
| | AACUUAAACAAAAGCUUGAAAACCUGCAGAAUUCUCAACUCCCCGAAAGC |
| | UUUAGAGUUCCAUAUGAUCCUGGACUGAAAGCAGGAGCGCUGGCAAUUGA |
| | AAAAUGUAAAGUAAUGGCCUCCAAGAAAAAACCACUAUGGCUUGAGUUUA |
| | AAUGUGCCGAUCCUACAGCCCUAUCAAAUGAAACAAUUGGAAUUAUCUUU |
| | AAACAUGGUGAUGAUCUGCGCCAAGACAUGCUUAUUUUACAGAUUCUACG |
| | AAUCAUGGAGUCUAUUUGGGAGACUGAAUCUUUGGAUCUAUGCCUCCUGC |
| | CAUAUGGUUGCAUUUCAACUGGUGACAAAAUAGGAAUGAUCGAGAUUGUG |
| | AAAGACGCCACGACAAUUGCCAAAAUUCAGCAAAGCACAGUGGGCAACAC |
| | GGGAGCAUUUAAAGAUGAAGUCCUGAAUCACUGGCUCAAAGAAAAAUCCC |
| | CUACUGAAGAAAAGUUUCAGGCAGCAGUGGAGAGAUUUGUUUAUUCCUGU |
| | GCAGGCUACUGUGUGGCAACCUUUGUUCUUGGAAUAGGCGACAGACACAA |
| | UGACAAUAUUAUGAUCACCGAGACAGGAAACCUAUUUCAUAUUGACUUCG |
| | GGCACAUUCUUGGGAAUUACAAAAGUUUCCUGGGCAUUAAUAAAGAGAGA |
| | GUGCCAUUUGUGCUAACCCCUGACUUCCUCUUUGUGAUGGGAACUUCUGG |
| | AAAGAAGACAAGCCCACACUUCCAGAAAUUUCAGGACAUCUGUGUUAAGG |
| | CUUAUCUAGCCCUUCGUCAUCACACAAACCUACUGAUCAUCCUGUUCUCC |
| | AUGAUGCUGAUGACAGGAAUGCCCCAGUUAACAAGCAAAGAAGACAUUGA |
| | AUAUAUCCGGGAUGCCCUCACAGUGGGGAAAAAUGAGGAGGAUGCUAAAA |
| | AGUAUUUUCUUGAUCAGAUCGAAGUUUGCAGAGACAAAGGAUGGACUGUG |
| | CAGUUUAAUUGGUUUCUACAUCUUGUUCUUGGCAUCAAACAAGGAGAGAA |
| | ACAUUCAGCCUAAUACUUUAGGCUAGAAUCAAAAACAAGUUAGUGUUCUA |
| | UGGUUUAAAUUAGCAUAGCAAUCAUCGAACUUGGAUUUCAAAUGCAAUAG |
| | ACAUUGUGAAAGCUGGCAUUUCAGAAGUAUAGCUCUUUUCCUACCUGAAC |
| | UCUUCCCUGGAGAAAAGAUGUUGGCAUUGCUGAUUGUUUGGUUAAGCAAU |
| | GUCCAGUGCUAGGAUUAUUUGCAGGUUUGGUUUUUUCUCAUUUGUCUGUG |
| | GCAUUGGAGAAUAUUCUCGGUUUAAACAGACUAAUGACUUCCUUAUUGUC |
| | CCUGAUAUUUUGACUAUCUUACUAUUGAGUGCUUCUGGAAAUUCUUUGGA |
| | AUAAUUGAUGACAUCUAUUUUCAUCUGGGUUUAGUCUCAAUUUUGGUUAU |
| | CUUUGUGUUCCUCAAGCUCUUUAAAGAAAAAGAUGUAAUCGUUGUAACCU |
| | UUGUCUCAUUCCUUAAAUGAUGCUUCCAAACAUCUCCUUAGUGUCUGCAG |
| | GUGUUAGUGGUGUGCUAAAAGCAAGGAAAGCGAGUUAGUCUUUUCAGUG |
| | UCUUUUGCAAUUCAAUUCUUUUGUCAUGUAUAACUGAGACACACAAACAC |
| | AGCAGGAGAAAUCUAAACCGUUGUGCCUUGACCUUCCUCUGCUGGUCUUG |
| | UUCCAGGGUUAUGAAUAUGAAAAAAUAGAGAUGAGACUUUUUGUGUCAA |
| | CUCUGUCCACAAGAGUGAGUUAUCUAGUAUGAUUAGUAUAGCUUUCUCCA |
| | GCAUGGCAGCAGGAAGUAACUACAGGGCCUCUUUUAUGCCUGACAUUUCU |
| | UCCCUUCCUUUUUCCCUGCCUCCCUUUUUCAUCAAUUGCGAUGCUCCCACA |
| | ACUCUUUACAGACUUGUGAAAUCUUCAAGAACACCUUUACUCUAUAACUC |
| | AAAAAUUAGUUGAAAAAUAAUUACUUCUCAAGGAUUAUUAGAAUCUUAG |
| | GUACUUAUUUGUAAAGAUGUUUAGUGACUUUUUUUUCAAGUAUCUUAUU |
| | AAAGGAGGCAUUCUAGAAAAUAUGAAUUAGUUUCCAAAUGCCUUAAUUUU |
| | AAACUUUGGCCUGAACAGUUUUUUCUUUUUCUUAAUGGAAGAAGAUAUUU |
| | AAUAUCUUAAAAAUAUUCCAAGUUAGGAAGAACACUACUUGCCUUAUCCA |
| | UUUCCCAUUUAAAGGACUUUUAAACUUUGACACAUCCUUCAGAUUUCCUG |
| | AAAAUAAUUGAAAUAUCUUACUUUAAAAAUAUUUUCAUCUCUGAAAUAUC |
| | UCGUUAUUUAUUGGAGGUAUUGUUUAACCUUAGAGAGACCAUUAAAUUA |
| | UUUAUAAAAUAUUUUGUAAUUACCUGUAGCUAAUACAUUACAUAGAAAA |
| | AAACUAUGUUAACAGUGUCUCUGUUUAAGUAUAAUCAGAUAUAAAUAUA |
| | UACUUAAUUUUUUAAUUUUAAAAAUAGAUACCUGUUUGACUUUGAGGUA |
| | GUCCAGACCUUUUCUUUUUUUUUUUUUUUUUAAUGUGUGCAAAAGCCCAA |
| | AGGUUCCUAAGCCUGGCUGCAAAGAAGAAUCAACAGGGACACUUUUUAAA |
| | AACACUCUUAUCAGCCUGGGCAACACAGUGAGACUCCAUCUCUUAAAAAA |
| | AAAAUUAGCUGGGUAUAGUGGUAUGUGCCUGUAGUCCCAGGUACUCAGGA |
| | GGCUGAGGCAGGAGGAUUGCCUGAGCCCAGGAGGUGGAAACUGCAGAGAG |
| | UCAUGAUCAUGUCCUUACACUCCAGCCUGGAUAACAGAGCGAGACCCUGU |
| | CUCAAAAAAAUAAAAUAAAAAAUAAAAACACCCUUGCCUGCGCUCCAUUC |
| | CCAGGUUAUAAUUUAAUUACUGUGGGAUGAGACCCAGACAUCAAUAUUUU |
| | UCUAGAUUCUUCAGGUGAUUCUAAUUCACAGCCAGAGUUGGAAGCCCUGC |
| | GUGGCCUUUGAAGGUCUAGAUGAUUCUUCUUCCUUGCCCUUUGAGCUUUU |
| | CCCCAUCUCACAGGUAUCUAGAAAAAAACUCCUCUUCUUUGGCAACCUGU |
| | CCUUUUAAAAUCACACUCUACCCACCUGUACAGAAGACCAUGCCCUAUAA |
| | UGAAAUGUUUAUUCCUAUCUAUAAAUGGAGGAUAAACAUUUUGUGGCACU |
| | UCUGGACCAACUAUUCCCUACUAUUCUUUUGAAGAAAGGCAGGAAGAGUA |
| | CUUUCUAAUUCAGAAGAGGAUGUUUUCACUAUUCUGAUAAACAAUAGCCA |
| | AGUUCAGACCUUGUACAGAUUCUUUUUAUUUGAAUUGCUGAAAUAAUUUA |
| | UUGAUGAUGAAAAAAAGAUUAGAGAGGAAUACAUUUAUAUUUAGCUUAU |
| | UGGCACAUGUGCAUACAUAUUUCCUCUUCAAAUGAACCAGUUCUUUCAUU |
| | UCAUUAUGCUAAUAUAUAUAUAUAACAUAUAUAUGCUAAUAUAUAUAUA |
| | ACAUAUAUAUGCUAAUAUAUAUAUAUCACACAUAUAUAUCACAGUUUUAU |
| | AUAUAUAUAUGUAUGUGUGUGUGUAUAUAUAUAUAUAUAUAUCACAGUU |
| | GGGCUUGAUUCUUCCGUAUUCCAAAGAGCAUAAUUUCAGUUCUAUAGACU |
| | UAUAGAUAAAUAAAAAUCAUCUUUGUGGGCUUCCUUCCUUUACUGUUCGC |
| | AGUGAAUUACAUGACGAACAACUUCUAUACCUUUGAAAAUGUUCUAGAAC |
| | UAGAAUCAUCCUGCUACUGGGAACUACCCACAGCUCUAUCUUCAAUGCCA |
| | GGUGAAAACACAGAUCACAAGUCAGAUGAAUCAGGCCAAAGCAACUUUUA |
| | UGUAUAUCUAGGACUGGCGACAUAAUUUGCAGAGCCCAGUGAACAGUGAA |
| | AAUGCAGUCCCAUCAUUCAAAAAUUAUUAAGAAAUUCGAGAUGGCAACAG |
| | CAGUUGUCACACCAAGCACAGUGCCCUUCUGAGCAUGAGGCUCUGUGUGA |
| | CUGCAUGGGUCCAUGAGACCAGCACUGUGUGGGUCUGAAUGAUGGCUUUG |
| | GUGUUCAGUUUAGCACACGCGGUCUACCACGUCUGCAUGAGUGGUAAAUG |
| | UAGUGCCUGGGUCAAAAUGUUCCUUCUGUUCAAAUCGAAAUACCUCAUCU |
| | CUAUGGCUCUAUGGCUGUACAUUAGGACCUAGAACAGUGGCCCAUUGCUC |
| | UUAGACUGGAACCAUGUCCACUAAAAUAAACCUAAGCAGAUGUUGUAGAC |
| | CUAGCCCCACAGGACUGCAUUUAGCUGCUUCAGUGACACUUUGAUGAAAG |
| | UAUGGAGAAGUGGAGACAUUAUAGAUAAAAUAUAUCAAUUCCCAGAGAA |
| | AACUCUUGACUUAAAAACUUAACUGUAGUAAAUAUAUCUUUUUCAGGUGA |
| | UGAAUUAUUUUUUUAAAAAAGGUUACAUAUAGGAAUUCUGCAGUAUAAU |
| | UUGGAGGCUAUUAGUGCUAUAUUAAUGGAAAUUAAUUAUUUUUUAAGUA |
| | AGUCCAAAAAAUAAUCUAGAAAGUAAGUUUCCAGAGCAAAUCUGACCUAG |
| | CAUUUGGUAUGCUAGGCUCUGCUUUUCAUGAUUUUGAAAUAAAUCAUAAU |
| | UAGACUUAACAAUAUGGAGAAAAUAAACUUGUAUUUUUAAGUGUUCUGU |
| | UGGCUUAUUUUCUGUUUCAUCCAACUCAAUAAUUCUGAUAAAUAAAUUUG |
| | GUUCUAGUUUGGUGCUUGUC (SEQ ID NO: 308) |
| FATS | AUUCUGAGCCUAGCCACAGAGGUAAACUCCGGACAUUUUUUUCUUUCAUG |
| NM_ | UCGCCAAAUACAUCCUUGUCAUUAAAAGACCUUUGAAACUGAGUAAAAUG |
| 001004298 | GCUUUGAAACCUCAGUAGAAUAUGCUCAAACUGUCUGGAGAAGGAUUACG |
| 118611 | GGACAGCUACCACAGUAGAAGAGAUCAAAUUGCCCUUAAAAAUCUCCAGU |
| | CUGAUGUCACAGAGGCAAAAUCUGACUUCACCAAGGAGACCCUGGCAUCA |
| | CAAAACACAAAAAUGAUUUCAUCCAUAGUCAUCUCCCAGAUGAUUGAUGA |
| | GAAUAAGUCAAGAGAAAACAGGGCCUCCUUGCCCCUACCGUGUGCCAUUG |
| | CUCAGUCUCGUGCACAUCACGCCAAGCAAUCUCUGGCUAACCGCAGCGGA |
| | GUCAACAUUCACAGAGCAUUUGCGUUACUUCCGGGCAGAUUAGGAAUCCC |
| | GGCACCGUCAGAUGAGCGAGGACCUGAGGCAGAGCUGCCGCCCAAAGAGG |
| | AGAGACCCUGUGGGGGCCCCCGCAGAGGGUUUGCAUCCAUCACCAUCACG |
| | GCCAGACGCGUGGGCCCCCCAGCCCGCGCCCUGGUGUGGGGGACUGCUGG |
| | GGACUCUCUGUGCCCCAAGUGCAGGGCUGAGGACACACUGUUCCAGGCGC |
| | CCCCGGCUCUGGCCAAUGGCGCCCAUCCAGGUCGGCAUCAGAGAUCUUUU |
| | GCCUGCACAGAGUUCUCCAGAAACAGCUCCGUGGUGCGGCUGAAGGUUCC |
| | CGAGGCCCACACUGGGUUGUGUGAGAGACGCAAGUACUGGGUCACCCAUG |
| | CAGACGACAAAGAGACCAGUUUUUCUCCAGACACCCCACUGUCAGGAAAG |
| | AGCCCGCUGGUGUUCAGUUCCUGUGUCCACCUCAGGGUGUCUCAGCAGUG |
| | UCCAGAUUCAAUCUAUUACGUAGACAAGUCUCUCUCCGUCCCCAUUGAGC |
| | CACCUCAAAUUGCCAGCCCCAAAAUGCACAGAUCCGUCCUGUCGCUCAACC |
| | UCAAUUGUAGUUCCCACAGAUUAACAGCAGAUGGAGUAGAUGGCCUAGUG |
| | AACAGAGAGCCAAUAAGCGAAGCCCUGAAGCAGGAGCUCCUGGAGGGAGA |
| | CCAGGACCUCGUAGGCCAGCGCUGGAACCCAGGUUUACAAGAAAGUCACU |
| | UGAAGGAAACCCCAUCGUUGAGGCGGGUGCAUUUGGGGACCGGCGCUUGU |
| | CCUUGGAGUGGUUCUUUUCCAUUGGAAAACACAGAAUUGGCAAAUGUGGG |
| | AGCUAACCAAGUCACUGUAAGAAAAGGGGAAAAGGACCAUACAACUCAUU |
| | GUCACGCCAGCGAUCAUGCCAACCAACUGUCCAUUCACAUUCCUGGCUGG |
| | AGUUACAGGGCAGUACACACAAAAGUAUUUUCUGGAAGCAGCAAGAGGCA |
| | ACAAGGAGAAGUAUGUAUGACUGUGUCUGCUCCUCCAGUGGAACAGAAGC |
| | CCACUAGACAUUUCCUUCCCAUUGGGGAUAGCUCUCCAAGCGAUGACUGU |
| | CUGUCUAGAGACCUUUCUGAGCCCACAGAGAGACGACAUCAAAGCUUCCU |
| | GAAACCCAGAAUCCUUUUUCCUGGGUUUCUUUGCCCCUUACAAGAUGUAU |
| | GUGCAUCUCUGCAGGAAGACAAUGGUGUUCAGAUAGAAAGCAAGUUCCCC |
| | AAAGGAGAUUACACAUGCUGUGACUUGGUUGUAAAAAUAAAGGAAUGUA |
| | AGAAGAGUGAGGACCCCACCACACCUGAGCCCUCCCCAGCAGCACCCUCGC |
| | CAGCACCCCGCGAUGGAGCAGGGAGCCCUGGCCUGUCCGAAGACUGUUCU |
| | GAGUCUCAGCAAACGCCUGCAAGAUCUUUGACCUUGCAAGAAGCACUGGA |
| | AGUUCGUAAGCCUCAGUUCAUUUCUCGCUCACAAGAACGGCUGAAGAAGC |
| | UUGAACACAUGGUCCAGCAGAGGAAAGCCCAGCGGAAGGAGGACCUGAGG |
| | CAGAAGCAGAGCCUCCUUCCCAUCCGCACCAGCAAGAAGCAGUUCACGAU |
| | UCCCCAUCCUCUGAGUGAUAACUUGUUCAAACCCAAAGAACGGUGCAUUU |
| | CAGAGAAGGAGAUGCAUAUGCGAUCUAAGAGAAUCUAUGAUAACUUGCCG |
| | GAAGUUAAAAAGAAAAAAGAAGAACAAAGGAAAAGGGUGAUCUUACAAA |
| | GUAACAGACUCCGUGCCGAAGUCUUCAAAAAGCAAUUACUGGACCAGCUC |
| | CUUCAAAGGAAUGCGGUCUAACCACAGGCUGCCCUGCUGACCACACUACC |
| | UGGACCUGGGAGGACUUCGAAUGCUGGAUAAGCCAUUAAACUUAGCAUUA |
| | UCUUCAUCAUGGGAAAACACUUAUUUUACUUUCGAUUUUUUUUUUCCAUA |
| | AAGGAAAACAUCACUUCCUGAAUUACUGACCCAAGCCAAACAGAAACAAU |
| | AACCCUCGAGAAAAUAGGUCAACAAUCUGUUGUGGGGAAACCACCUCCAU |
| | GUAACCCACUGGAUACAGUCCCCUUAAUGUUUUUGCUUCUAAAUUGUACC |
| | UUUUGCUUCUGAUUUCUUCUCCCCUGCUGUUUCCUGCCCAUCAGAGAGGC |
| | CUGAUACAAGCAAGUUUGUUUACAUCCCUGGGGAAUCUUUUACAUCAAAC |
| | UUUUGGGAUCCAAAUCCAUCUCCUUUUAAAUUUCAAUCUCAGCACCUGCC |
| | AGUUAUGCAAAUGCCAAAAUGUGGGUCAUCAUAUAGUAUAUUUGAAACCU |
| | UUCUGAACAUGUACACCACCCAAUGCUAGAGGCUGACUUGGAAACCGGUG |
| | GGUGCAAUGCCCGAGGCUGUGGAACAAUCAGCCCAUCUCUUUGUGACCUG |
| | GAGCAGUCAGAGGGCCCUCAGUCACCCCGCCCCAUUCCUCACCAGAGAAAA |
| | GAAUCCAUUUCUAUGGAAGCUCUGACGUAACUUCAGUGUUUUCUACAAUA |
| | CUCCUCCUGCCCCGCCCCAUUAAAACAGUUCUUUUGUUAAGAAAUAGCCU |
| | AAUGGUCCAACUUUGCUGUCUGUUCUUCCAAAUGUUUAUAAUACACAUUA |
| | UUUAUAAAUAUGUCUGUUUGGGAAGCUAAGAACAAGCUAGUUUUUACAAC |
| | ACAAAUGGAAAUAAAUGCAAUUAUUAUAAAAAUUCAA (SEQ ID NO: 309) |
| PTGS2 | AAUUGUCAUACGACUUGCAGUGAGCGUCAGGAGCACGUCCAGGAACUCCU |
| NM_ | CAGCAGCGCCUCCUUCAGCUCCACAGCCAGACGCCCUCAGACAGCAAAGCC |
| 000963 | UACCCCCGCGCCGCGCCCUGCCCGCCGCUGCGAUGCUCGCCCGCGCCCUGC |
| 5743 | UGCUGUGCGCGGUCCUGGCGCUCAGCCAUACAGCAAAUCCUUGCUGUUCC |
| | CACCCAUGUCAAAACCGAGGUGUAUGUAUGAGUGUGGGAUUUGACCAGUA |
| | UAAGUGCGAUUGUACCCGGACAGGAUUCUAUGGAGAAAACUGCUCAACAC |
| | CGGAAUUUUUGACAAGAAUAAAAUUAUUUCUGAAACCCACUCCAAACACA |
| | GUGCACUACAUACUUACCCACUUCAAGGGAUUUUGGAACGUUGUGAAUAA |
| | CAUUCCCUUCCUUCGAAAUGCAAUUAUGAGUUAUGUGUUGACAUCCAGAU |
| | CACAUUUGAUUGACAGUCCACCAACUUACAAUGCUGACUAUGGCUACAAA |
| | AGCUGGGAAGCCUUCUCUAACCUCUCCUAUUAUACUAGAGCCCUUCCUCC |
| | UGUGCCUGAUGAUUGCCCGACUCCCUUGGGUGUCAAAGGUAAAAAGCAGC |
| | UUCCUGAUUCAAAUGAGAUUGUGGAAAAAUUGCUUCUAAGAAGAAAGUUC |
| | AUCCCUGAUCCCCAGGGCUCAAACAUGAUGUUUGCAUUCUUUGCCCAGCA |
| | CUUCACGCAUCAGUUUUUCAAGACAGAUCAUAAGCGAGGGCCAGCUUUCA |
| | CCAACGGGCUGGGCCAUGGGGUGGACUUAAAUCAUAUUUACGGUGAAACU |
| | CUGGCUAGACAGCGUAAACUGCGCCUUUUCAAGGAUGGAAAAAUGAAAUA |
| | UCAGAUAAUUGAUGGAGAGAUGUAUCCUCCCACAGUCAAAGAUACUCAGG |
| | CAGAGAUGAUCUACCCUCCUCAAGUCCCUGAGCAUCUACGGUUUGCUGUG |
| | GGGCAGGAGGUCUUUGGUCUGGUGCCUGGUCUGAUGAUGUAUGCCACAAU |
| | CUGGCUGCGGGAACACAACAGAGUAUGCGAUGUGCUUAAACAGGAGCAUC |
| | CUGAAUGGGGUGAUGAGCAGUUGUUCCAGACAAGCAGGCUAAUACUGAUA |
| | GGAGAGACUAUUAAGAUUGUGAUUGAAGAUUAUGUGCAACACUUGAGUG |
| | GCUAUCACUUCAAACUGAAAUUUGACCCAGAACUACUUUUCAACAAACAA |
| | UUCCAGUACCAAAAUCGUAUUGCUGCUGAAUUUAACACCCUCUAUCACUG |
| | GCAUCCCCUUCUGCCUGACACCUUUCAAAUUCAUGACCAGAAAUACAACU |
| | AUCAACAGUUUAUCUACAACAACUCUAUAUUGCUGGAACAUGGAAUUACC |
| | CAGUUUGUUGAAUCAUUCACCAGGCAAAUUGCUGGCAGGGUUGCUGGUGG |
| | UAGGAAUGUUCCACCCGCAGUACAGAAAGUAUCACAGGCUUCCAUUGACC |
| | AGAGCAGGCAGAUGAAAUACCAGUCUUUUAAUGAGUACCGCAAACGCUUU |
| | AUGCUGAAGCCCUAUGAAUCAUUUGAAGAACUUACAGGAGAAAAGGAAAU |
| | GUCUGCAGAGUUGGAAGCACUCUAUGGUGACAUCGAUGCUGUGGAGCUGU |
| | AUCCUGCCCUUCUGGUAGAAAAGCCUCGGCCAGAUGCCAUCUUUGGUGAA |
| | ACCAUGGUAGAAGUUGGAGCACCAUUCUCCUUGAAAGGACUUAUGGGUAA |
| | UGUUAUAUGUUCUCCUGCCUACUGGAAGCCAAGCACUUUUGGUGGAGAAG |
| | UGGGUUUUCAAAUCAUCAACACUGCCUCAAUUCAGUCUCUCAUCUGCAAU |
| | AACGUGAAGGGCUGUCCCUUUACUUCAUUCAGUGUUCCAGAUCCAGAGCU |
| | CAUUAAAACAGUCACCAUCAAUGCAAGUUCUUCCCGCUCCGGACUAGAUG |
| | AUAUCAAUCCCACAGUACUACUAAAAGAACGUUCGACUGAACUGUAGAAG |
| | UCUAAUGAUCAUAUUUAUUUAUUUAUAUGAACCAUGUCUAUUAAUUUAA |
| | UUAUUUAAUAAUAUUUAUAUUAAACUCCUUAUGUUACUUAACAUCUUCUG |
| | UAACAGAAGUCAGUACUCCUGUUGCGGAGAAAGGAGUCAUACUUGUGAAG |
| | ACUUUUAUGUCACUACUCUAAAGAUUUUGCUGUUGCUGUUAAGUUUGGAA |
| | AACAGUUUUUAUUCUGUUUUAUAAACCAGAGAGAAAUGAGUUUUGACGUC |
| | UUUUUACUUGAAUUUCAACUUAUAUUAUAAGAACGAAAGUAAAGAUGUU |
| | UGAAUACUUAAACACUGUCACAAGAUGGCAAAAUGCUGAAAGUUUUUACA |
| | CUGUCGAUGUUUCCAAUGCAUCUUCCAUGAUGCAUUAGAAGUAACUAAUG |
| | UUUGAAAUUUUAAAGUACUUUUGGUUAUUUUUCUGUCAUCAAACAAAAAC |
| | AGGUAUCAGUGCAUUAUUAAAUGAAUAUUUAAAUUAGACAUUACCAGUA |
| | AUUUCAUGUCUACUUUUUAAAAUCAGCAAUGAAACAAUAAUUUGAAAUUU |
| | CUAAAUUCAUAGGGUAGAAUCACCUGUAAAAGCUUGUUUGAUUUCUUAAA |
| | GUUAUUAAACUUGUACAUAUACCAAAAAGAAGCUGUCUUGGAUUUAAAUC |
| | UGUAAAAUCAGUAGAAAUUUUACUACAAUUGCUUGUUAAAAUAUUUUAU |
| | AAGUGAUGUUCCUUUUUCACCAAGAGUAUAAACCUUUUUAGUGUGACUGU |
| | UAAAACUUCCUUUUAAAUCAAAAUGCCAAAUUUAUUAAGGUGGUGGAGCC |
| | ACUGCAGUGUUAUCUUAAAAUAAGAAUAUUUUGUUGAGAUAUUCCAGAA |
| | UUUGUUUAUAUGGCUGGUAACAUGUAAAAUCUAUAUCAGCAAAAGGGUCU |
| | ACCUUUAAAAUAAGCAAUAACAAAGAAGAAAACCAAAUUAUUGUUCAAAU |
| | UUAGGUUUAAACUUUUGAAGCAAACUUUUUUUUAUCCUUGUGCACUGCAG |
| | GCCUGGUACUCAGAUUUUGCUAUGAGGUUAAUGAAGUACCAAGCUGUGCU |
| | UGAAUAAUGAUAUGUUUUCUCAGAUUUUCUGUUGUACAGUUUAAUUUAG |
| | CAGUCCAUAUCACAUUGCAAAAGUAGCAAUGACCUCAUAAAAUACCUCUU |
| | CAAAAUGCUUAAAUUCAUUUCACACAUUAAUUUUAUCUCAGUCUUGAAGC |
| | CAAUUCAGUAGGUGCAUUGGAAUCAAGCCUGGCUACCUGCAUGCUGUUCC |
| | UUUUUUUUCUUCUUUUAGCCAUUUUGCUAAGAGACACAGUCUUCUCAUC |
| | ACUUCGUUUCUCCUAUUUUGUUUUACUAGUUUUAAGAUCAGAGUUCACUU |
| | UCUUUGGACUCUGCCUAUAUUUUCUUACCUGAACUUUUGCAAGUUUUCAG |
| | GUAAACCUCAGCUCAGGACUGCUAUUUAGCUCCUCUUAAGAAGAUUAAAA |
| | GAGAAAAAAAAAGGCCCUUUUAAAAAUAGUAUACACUUAUUUUAAGUGA |
| | AAAGCAGAGAAUUUUAUUUAUAGCUAAUUUUAGCUAUCUGUAACCAAGAU |
| | GGAUGCAAAGAGGCUAGUGCCUCAGAGAGAACUGUACGGGGUUUGUGACU |
| | GGAAAAAGUUACGUUCCCAUUCUAAUUAAUGCCCUUUCUUAUUUAAAAAC |
| | AAAACCAAAUGAUAUCUAAGUAGUUCUCAGCAAUAAUAAUAAUGACGAUA |
| | AUACUUCUUUUCCACAUCUCAUUGUCACUGACAUUUAAUGGUACUGUAUA |
| | UUACUUAAUUUAUUGAAGAUUAUUAUUUAUGUCUUAUUAGGACACUAUG |
| | GUUAUAAACUGUGUUUAAGCCUACAAUCAUUGAUUUUUUUUUGUUAUGUC |
| | ACAAUCAGUAUAUUUUCUUUGGGGUUACCUCUCUGAAUAUUAUGUAAACA |
| | AUCCAAAGAAAUGAUUGUAUUAAGAUUUGUGAAUAAAUUUUUAGAAAUC |
| | UGAUUGGCAUAUUGAGAUAUUUAAGGUUGAAUGUUUGUCCUUAGGAUAG |
| | GCCUAUGUGCUAGCCCACAAAGAAUAUUGUCUCAUUAGCCUGAAUGUGCC |
| | AUAAGACUGACCUUUUAAAAUGUUUUGAGGGAUCUGUGGAUGCUUCGUUA |
| | AUUUGUUCAGCCACAAUUUAUUGAGAAAAUAUUCUGUGUCAAGCACUGUG |
| | GGUUUUAAUAUUUUUAAAUCAAACGCUGAUUACAGAUAAUAGUAUUUAU |
| | AUAAAUAAUUGAAAAAAAUUUUCUUUUGGGAAGAGGGAGAAAAUGAAAU |
| | AAAUAUCAUUAAAGAUAACUCAGGAGAAUCUUCUUUACAAUUUUACGUUU |
| | AGAAUGUUUAAGGUUAAGAAAGAAAUAGUCAAUAUGCUUGUAUAAAACA |
| | CUGUUCACUGUUUUUUUUAAAAAAAAAACUUGAUUUGUUAUUAACAUUG |
| | AUCUGCUGACAAAACCUGGGAAUUUGGGUUGUGUAUGCGAAUGUUUCAGU |
| | GCCUCAGACAAAUGUGUAUUUAACUUAUGUAAAAGAUAAGUCUGGAAAUA |
| | AAUGUCUGUUUAUUUUUGUACUAUUUAAAAAUUGACAGAUCUUUUCUGA |
| | AGAUAAACUUUGAUUGUUUCUAUA (SEQ ID NO: 310) |
| PPARa | GUGGACGCGGCGGCCCCGCGGCGGGGGCAGCGGGCGGCGGGGGCGGAGGC |
| NM_ | GGCCGCUAGCGCCCUGCCCGGCGCCGCCUCCUUCGGCGUUCGCCCCACGGA |
| 001001928 | CCGGCAGGCGGCGGACCGCGGCCCAGGCUGAAGCUCAGGGCCCUGUCUGC |
| 5465 | UCUGUGGACUCAACAGUUUGUGGCAAGACAAGCUCAGAACUGAGAAGCUG |
| | UCACCACAGUAGCUUGGAGCUCGGCGGCACAACCAGCACCAUCUGGUCGC |
| | GAUGGUGGACACGGAAAGCCCACUCUGCCCCCUCUCCCCACUCGAGGCCGG |
| | CGAUCUAGAGAGCCCGUUAUCUGAAGAGUUCCUGCAAGAAAUGGGAAACA |
| | UCCAAGAGAUUUCGCAAUCCAUCGGCGAGGAUAGUUCUGGAAGCUUUGGC |
| | UUUACGGAAUACCAGUAUUUAGGAAGCUGUCCUGGCUCAGAUGGCUCGGU |
| | CAUCACGGACACGCUUUCACCAGCUUCGAGCCCCUCCUCGGUGACUUAUCC |
| | UGUGGUCCCCGGCAGCGUGGACGAGUCUCCCAGUGGAGCAUUGAACAUCG |
| | AAUGUAGAAUCUGCGGGGACAAGGCCUCAGGCUAUCAUUACGGAGUCCAC |
| | GCGUGUGAAGGCUGCAAGGGCUUCUUUCGGCGAACGAUUCGACUCAAGCU |
| | GGUGUAUGACAAGUGCGACCGCAGCUGCAAGAUCCAGAAAAAGAACAGAA |
| | ACAAAUGCCAGUAUUGUCGAUUUCACAAGUGCCUUUCUGUCGGGAUGUCA |
| | CACAACGCGAUUCGUUUUGGACGAAUGCCAAGAUCUGAGAAAGCAAAACU |
| | GAAAGCAGAAAUUCUUACCUGUGAACAUGACAUAGAAGAUUCUGAAACUG |
| | CAGAUCUCAAAUCUCUGGCCAAGAGAAUCUACGAGGCCUACUUGAAGAAC |
| | UUCAACAUGAACAAGGUCAAAGCCCGGGUCAUCCUCUCAGGAAAGGCCAG |
| | UAACAAUCCACCUUUUGUCAUACAUGAUAUGGAGACACUGUGUAUGGCUG |
| | AGAAGACGCUGGUGGCCAAGCUGGUGGCCAAUGGCAUCCAGAACAAGGAG |
| | GCGGAGGUCCGCAUCUUUCACUGCUGCCAGUGCACGUCAGUGGAGACCGU |
| | CACGGAGCUCACGGAAUUCGCCAAGGCCAUCCCAGGCUUCGCAAACUUGG |
| | ACCUGAACGAUCAAGUGACAUUGCUAAAAUACGGAGUUUAUGAGGCCAUA |
| | UUCGCCAUGCUGUCUUCUGUGAUGAACAAAGACGGGAUGCUGGUAGCGUA |
| | UGGAAAUGGGUUUAUAACUCGUGAAUUCCUAAAAAGCCUAAGGAAACCGU |
| | UCUGUGAUAUCAUGGAACCCAAGUUUGAUUUUGCCAUGAAGUUCAAUGCA |
| | CUGGAACUGGAUGACAGUGAUAUCUCCCUUUUUGUGGCUGCUAUCAUUUG |
| | CUGUGGAGAUCGUCCUGGCCUUCUAAACGUAGGACACAUUGAAAAAAUGC |
| | AGGAGGGUAUUGUACAUGUGCUCAGACUCCACCUGCAGAGCAACCACCCG |
| | GACGAUAUCUUUCUCUUCCCAAAACUUCUUCAAAAAAUGGCAGACCUCCG |
| | GCAGCUGGUGACGGAGCAUGCGCAGCUGGUGCAGAUCAUCAAGAAGACGG |
| | AGUCGGAUGCUGCGCUGCACCCGCUACUGCAGGAGAUCUACAGGGACAUG |
| | UACUGAGUUCCUUCAGAUCAGCCACACCUUUUCCAGGAGUUCUGAAGCUG |
| | ACAGCACUACAAAGGAGACGGGGGAGCAGCACGAUUUUGCACAAAUAUCC |
| | ACCACUUUAACCUUAGAGCUUGGACAGUCUGAGCUGUAGGUAACCGGCAU |
| | AUUAUUCCAUAUCUUUGUUUUAACCAGUACUUCUAAGAGCAUAGAACUCA |
| | AAUGCUGGGGGUAGGUGGCUAAUCUCAGGACUGGGAAGAUUACGGCGAAU |
| | UAUGCUCAAUGGUCUGAUUUUAACUCACCCGAUGUUAAUCAAUGCACAUU |
| | GCUUUAGAUCACAUUCGUGAUUUACCAUUUAAUUAACUGGUAACCUCAAA |
| | AUUCGUGGCCUGUCUUCCCAUUCACCCCGCUUUUGACUAUUGUGCUCCUU |
| | UAUAAUUCUGAAAACUAAUCAGCACUUUUUAACAAUGUUUAUAAUCCUAU |
| | AAGUCUAGAUGUAUCCAAAGGUGAAGUAUGUAAAAAGCAGCAAAAUAUU |
| | UAUUUCAAAGACUUCACUUCUGUUUCCUGAAUCUAAAGAAAGACAACAUG |
| | CUGCUUUUUAAUCAUAGGAUGGAGAAUUUUAAAGAACUGUUUGGGCCAGG |
| | CACAGUCGCUCAUACUUGUAAUCCCAGCACUUUGGGAGGCCGAGGCGGGU |
| | GGAUCACAAGGUCAGCAGAUCGAGACCAUCCUGGCCAACAUGGUGAAACC |
| | CUGUCUCUACUAAAAAUACAAAAAUUAGCCGGGUGUGGUGGCACAUGCCU |
| | GUAAUCCCAGCUACUCGGGAAGCUGAGGCAGGAGAAUUGCUUGAACCAGG |
| | GAGUUGGAGGUUGCAGUGAGCUAAGACUGCACCACUGCACUCCAGCCUGG |
| | UGACAGAACGAGACUCUGUCUUAAAAACAAACAAACAAAAAAAAAAUCUG |
| | UUAGAUAAGCUAUCAAAAUGCAGCUGUUGUUUUGUUUUUGGCUCACUGUU |
| | UUCGUGGUUGUAACUAAUAUGUGGAAAGGCCCAUUUCCAGGUUUGCGUAG |
| | AAGAGCCCAGAAAACAGAGUCUCAAGACCCCCGCUCUGGACUGUCAUAAG |
| | CUAGCACCCGUGGUAAGCGGGACGAGACAAGCUCCCGAAGCCCGCCAGCU |
| | UCCUGCUCCACUCAGCUCCGUCCAGUCAACCUGAACCCACCCAGUCCAGCU |
| | GUCUGUGGGAAUGGUGGUGUUCUUAGGGACAGACUGACACCUUACUUGUC |
| | AGUGUUCCUCCGGGCCCCAUUUGGCAGCUCCCGUAUCUUUUGUUAUGUUG |
| | CUUUUAAAGAUAUGAUGUUUUAUUGUUUUAACUCUUGGUGACAGUAGAU |
| | GCUCUCUGGAGCGCAGACGAGGCACAUGUGUCUUCAUAGCCUGGGCUGGG |
| | UGGGAGCCAGUCACCCUGCGGAUCGAGAGAGGGGGUAGAGUCUUCUUCAA |
| | AUGGCAGUUUUACUUCAAAUGGCAGAUUUCACAAGAGUUGGUUAUUUUUU |
| | ACAAUGGUUUAGGUUGUUAAGUCUCCUUUGUAUGUAAGGUAGUUUUUUC |
| | AACAUCUAAAAUUUUUGUUUUAGCCUUCAAAACCAACUUACCAACCUCAG |
| | UCCAGCUGGGAAGGCAGCGUUGAUUAUGGUAGUUUGUCAAGAAUAUAUGG |
| | ACCUGGAAACACUUUCUCUCUCUGUCCACCUGGUAGAUAAAUUGUCCUGU |
| | UGAGAAUUUUUAGAUCUGGACUGGAACUGCCAGGACCACCGCCUCCAGGG |
| | AGUCGCUGGGCACCUGGAGGUAUCGUCGAUGCCUCUCCCCCAUCUUUAGA |
| | AAAUUUGGCUCUUCUGAGGUCAUUAUUAUUUUAAGAAUGAUUAGGAUUG |
| | AUAAGGGUCCCAUGACCAGCAUUAUGAAAAUGCGAGAGUGGGAAGGACAC |
| | AGUGUGAGACUUCCACUAGAAAAAAGUGAAAGUUAGGGUUAGGACAUCCU |
| | UUUUUAAAAAUUACAAAUUUAGUCCGUUUUGGUUUUUGUAAUCAGGCUA |
| | GGCACAGUGGCUCACACAUGGAAUCCCAGCACUUUGGGAGGCCGAGGUGG |
| | GAGGAUCACUUGAGCCCAGGAGUUCGAGACCAGCCUAGGCAACAUAGCAA |
| | GACCCUGUCUGUACACAAAAUUUAAAAAUUAGUUCAUCGGGGUGGCACAC |
| | AUCAGUAGUCCCAGCUACUCUGCAGGCUGAGGUGGGAGGAUUGCUUGAAC |
| | CCAGGAGGUCGAGGCUGCAGUGAGCUGUGAUCUCACCACUGCAUUCCAGC |
| | CUGGGUGACAGAGUUAGAUUCCACCCUCUCCCACCCCGGCAAAAAAAAAA |
| | AAAAAAGAUGCAAUCAAAGGGGCUGUUGGCCAGCAAUGGCAGCAGCAGCG |
| | GCGGGCAGUCUGCCCAAGUGUCUUAGGAACCAAAAGCAAAUAAAAGUGUU |
| | UCCAUAUAUGCCACCAGCCAAGUGGCCAUCCUAAUUCAGAAAGAAGCUAG |
| | CCUUUGAGUGUCUGUCAUGGUGCAUCCGUUUCAGUAUUAUUUCCUAAAAU |
| | GAGAAGCCCCUGUGUCAACAAGAUCCAGGGGCUGGAGCCCAAUGCCAAGC |
| | CUGUGUUGUCCCCAGCGACCCUGCAGCUGCUCGCUCUGAUGUACCCUGUG |
| | CCAUUCAAGGAGAUGUGGUCCAGGAAAGUGAGCCUCAUGGUUUUCAGAGA |
| | AGUCAUUGUUCUGUUUACAUUUUCAUAAAACCUGUUUAAAAUAGCUCCCC |
| | GUCUCAGGCUUUCAGCAGUAACAGUGAGCUGACUGGCAAGUUCGAUGUUA |
| | GCUCCCGGGACACUCAGCAGCGAUGGUGAGCAUUUUGGUUUCCUUAAGGC |
| | CCAGCAAGACUUCCAGGGACAUCUCUGGUGAAGCCAGAAUGGAGACACCC |
| | GUGACCUCAGGCUGAAAGUCACUCGACAUUGGUCUCUUGUGUUGAUAGGG |
| | AAGGAAAUCAGGCAUUCCUAUUUCUUUAAAUAACAAAACCACUAAUUGCC |
| | ACUCAAUGCUGGAAUAUUUUGGGUCACCUAAUCAUAGAUUUCUCAGGGCA |
| | UCAAUACUCAAAUAUAGGCUGAUUAUGCCCCAGUUCAAAUGGGAACUAUU |
| | AACAGAGUGCAUUUCUUGCUUGCUGGGUUUCAACAGACAUCAGCCAAAAG |
| | AACAAAAGAGAUGUCAGGACAGAUUCCAGGAGUGUCGGAGCACAUGUGUG |
| | GCACCCGCUCCCUCUGGCAGCGAAUGUAGGAAGUCGCCAAAUUUACCCAC |
| | UCUUCAACAAGUCAUUGUUUAAACACGGUUUUUCAUUUUCUCAACUUUUA |
| | AUAGCAAAAAGUGCCAAAGUCCUCAGAGACCUAACAGCCUUGGUCUACCG |
| | UGCUGACCAGGGUGAAGGCACGGCGAGGGACUCCUCCCAGACGUGCCUCU |
| | UGUGUGCCAGCUGGCUGUGGCUCGGGAGCAGACGCAGGCCUCUCCAUUGU |
| | CCAGGGGAGCCUGGCGGCGCAUCCCUCCUCUCCCACCUCCUGGCACUUCCA |
| | GCUGGGUGUCCCACAUGUUGGAUUCCGUCCCCACCACACUUCCAGAGACC |
| | GGAGAACUGUGCAGGGCCUAAGGCCGUUUGGAUGAAUUGUCAAAACAAGA |
| | UGCUUCCAGUUACAGCGGCAGGAGCGGGACUGGGAGCACGGGCUGACGGC |
| | UGCUGGUGCCUUUCUUCCCACCUCGCUUGCCUGUUUCCGCUUGACCCUUCC |
| | UCCAGCUCCGAUGAGAAGAGUAUAAAGCAUCUUCCUAACGGGUGUGUUUG |
| | CUAUACGAACAUAAUGGACGUGAAGUGGGGCAGAAACCCAGAACUCAGCA |
| | UUCAAGGAUGCCCAGGAGAGCUGUCCCUGUUUUAAAGAGCUGUGUUUUGU |
| | UUUGUUUCGCAUUUAGAGAGCAGACAAGGCACCCUUCUGCUGCGCUGAUA |
| | CGUUUCUUACACUGGGCCAUUUUAGACCCCCAGGGAAACAGCCUUCCUGG |
| | AGCGUUGUCUGGAGGUUCCAGGGACAGGGCAGCCUCCCAGAGCCGAGCAA |
| | GAGCUCAAGGUACAAAUGAGAGAUUUGCUAUACCGUGAGAAGUCAACAAC |
| | UUAGCCACCACUUCCCCGCAAUGGACCAUGUAACAAAUACCUCAGCAGGC |
| | CCUGCAAAAGGCCAUGCUAGAGCUGAGGCGCACAGCCUGUGGCCUCUGUA |
| | GUUAGGGCAGGUGGGAUGGAGACUCCUUGAGUGCACACACCUGAGCCUGC |
| | CCACACACAGGGGAGCAGCAUCUCGUAUGACGUCUGGAAGGAACUUCGGU |
| | UGUGUAAAGGGAGCCUUGAAGAUACGUGCAAAAGGUGCUACCCCAAUUUG |
| | GUGAAACUGACAUUGGGCACGUCUUGGGCUUAGGAGAAGCGGCCGAUGGU |
| | CCCGGCCUGCAGUGACAAACCCCCCUCCCCGCACCGCCCCCAGCACCCCCU |
| | CUCCUCUUCACCUCUUCCUGCUGGCCACGAGGAAGCCACUUCCUCAGAGAG |
| | ACCCUACCAGAUGCGGAUGGAAACAGAUGCACCAAAGCAAGCCCUGAUGA |
| | AACCGCGACUUCCUAAGGUCUGUCUCCUCUGAACUUGCACCUGGGCCUCU |
| | CUGUGUUUGGUUCCAAGCACUUCCCACCUCAAACUCCCAUUUUCAAACCA |
| | CUGUAUCUCUGCGCACAUCUGCUACUUACCAGCCGCAUACAUGAUGGAGG |
| | GUUUUUUGGUCCUGAUCCAGUGGCCACACCUGUCUUUGAAAUGUCUCACU |
| | GAACUCCAGUUUUAAAAUAGAUUCAUUGCUUCAACACAGCAAGCCCAAUG |
| | CACCCAGCUAAGACUGGCUUGACCGACAGCCUGGCCUUUGGUGGGGGGCU |
| | UCCUGGGGCCUGGGGAAAGCUGGCCACCUUCAACAGCUGGUACCUCUUCA |
| | ACAGUGUGGCCUUUCAAAAUGCAGAUGCCACCAGGAGAACAUGCCCACAG |
| | CUCACCACCUAUGGAUGCCAUGGCUCUGGGCAGCUUUCAAAGCAGGUUCC |
| | UGUGGUCUCCUCAGCUGUUUGAGGGGGUAACAGCAAAUCAGCCUCCAUUU |
| | UAAAAUGAAAACACCAGCCUCCAGAUGUAGGGCCUGCUGGGUGUUGCUAG |
| | CCGCUGGUCCCCAGGCACGGUGCACUUUCUCCACCUCCUGCAGCCUCCCUG |
| | UUGUUUCUAGACUCUUGCACCUGGUGAGUGCAAGGAUAGGUGACCCAGGG |
| | GCCUGCAGCCUUGUCCUCAGCUCCCAUCUCCUGGACUGCCAGCCUCACCCU |
| | CUGCAGUUAGCAUGGUUGGCCUGAUGCAGGGAUCCCGAGGGAUUACUUUU |
| | UAGACCUUCUUUCACAUUCAGAAAAGUAGUAUAGAUUCAGGAGAGGCAAG |
| | AAAAUUAUGCUGUCCAUAGAAGUCACCCAUGAAGACUGAUGCCACCACCU |
| | GAAGGCUCAUGAUUGUUAAAAAUGUCCACGGGAACCUCUCGUCCACAGGA |
| | GGUUUGUCUCAACACUUCCCAUUUUUACGGCAUUGGCAUUGCCAAGCAUG |
| | GGGAAGUAUCUGCUCUUCUCAUGUUAAAAGUGGCCCAGCUUUUCUUAACU |
| | CAGUCCAAGCUGACUUGUUUAGCUGCACUGGAAUUUCUUACCAACCAAAU |
| | AUUUGCAUCGAGCAAAGGGGGCUGUGUGCACCUCCCUAAUGGCAGCGAUG |
| | AUGGCUGCUGUCAUUCAAGCCCAUCUUCAGACGUCACAGUCUGGAAGUGA |
| | AAUGUCCACAAACAUCUGUGGCAGAAAAGGCUAUACGGACCACCCAGUUG |
| | UGCUGCAGCUUUACAGAGCAAGGAAGGGUUGUGGCAAAUAAAUGAUUAAC |
| | CUGCCUCGACUGUGCUGAGGGCAACAAAGGCCAUCUCACCAAAGGAUUAU |
| | UCGAUGCCAUUAAAUCAUCCCGUGACCUUCCUGCUUCCGAGUCCAUGGCC |
| | UUUGCCCAGGGCAUGUACUCCCCUGAGAGGCCUUCUGCCUAGAAAGAUCU |
| | AUGACUGGGUUCCAAAGUUGAGGCCUAGGUUUUUGCUGGGAUUUAGAUAU |
| | UUUCAGGCACCAUUUUGACAGCAUUCAGGAAAACGGUUAUUGACCCCAUA |
| | GACUAGGGUAAGAAUAAAGGCAAUAAAUUUGGUCUGACUCAGAAUAUAG |
| | GAGAUCCAUAUAUUUCUCUGGAAACCACAGUGUACACUAAAAUGUGAAAU |
| | UGAAGGUUUUGUUAAAAAGAAAAAGAUAAUGAGCUUCAUGCUUUGUUUA |
| | AUUACAUAAUGAUUUCCAUUACGCUAUUUCUGUGAAAUGCAGCAGGUUCU |
| | UAAACGUUAUUUCAGUGGCAUGGGCUGGAAGCUUAUCACAAAAAGCCAUG |
| | UGUGUGGCCUUAUCAGAACAGAAAGAGACAGGCUGGUGCCCAAGGCUGCU |
| | GCCUGCUCCACCUUUUGCCAGCUCUGGACAUCUGAGGACGUCCCGGCAGA |
| | UCUGGAAUGGGGCCCUCAACUGACCAUUUGCUUCUCAGAAUUUCAGUUUG |
| | AGACAUGAGAGGUAUAAUCAGUUACUUUUCUCCCCCCAGAGAAACCCUUU |
| | UGUGAGGGGAGAGGAGCUAUGGUAUGUGGUUCAGCUGAAACACAUACAAC |
| | UGCAUCCUUUUGGAGUCCUUUGCCAACAAAAACAGACCAACAGACCAGAU |
| | GGUGUCCAUGUUCAAUAUCAUGUCUUGAUGGACGCAGCUGAUGACCUCAA |
| | AUACUUGAGUGGUCUCAUGGCUGUUAGAUGGAUUAUUUGAAAAAAAAAA |
| | AAAAAAAAGAGAGAAAAAAUAAUUGAUUUUUACAUCAGAGAUAGCAAAC |
| | UAAGACCUGGGGAGGGGGGUCAGCUUUUAUUUUAUUUUAUUUUUUUUAA |
| | GUUUGCUAGUUGGGUCAAAUGUGAGGAGGAGGGAGUCUACCUGCCACCUC |
| | UUCUCUUGCCCCUCUUCUGCCCACACAUCCAGCAUCCAAAAUCCAUUCAUU |
| | UAAUGAAUUGAUAAAGUGCCGUGCAAACUGGUGCACAAACAGGCCCCCAG |
| | UCCACGCAGCCUGGCUCCUAGGAAAAGUGGUGACCGGGCGUGGGGGGGCA |
| | UGCCGCAGCCCUGGGACACAGUCGGGCACCUUCCCCGGACCCCCAGGCCUU |
| | GGCUGUGCCUCAAGUCAGAGAGGGUCAGCCUUCAGGCCCCGGAGACGAGU |
| | GACUGGCCGAUCAUUUCACAAUAAAAUCACUCACUUUUGGCAACUUCACU |
| | UUUUUUAAGGCACAGUCAGUUCCUUUUCUCAUGUACCUCACAAAAGAUGA |
| | AGACCAUGUAGUACUCUUUUUGGUAAAGUUACAGUGUUCAUGUUAAAUAU |
| | CACUUUUUUCUACAUUGUGUGGUAAAAAGAACUACGUUAAUAGCUAUAUC |
| | UUAAAUACUGUGAUUUGACUUUUUGAAAAAUAUCCUAAUACAAAUAUUU |
| | UACUAACUUACAAUCACUCAUUUAAUAAGAAACAUUUGGAUUCUUUUGAA |
| | AUCAGUGUUAAUUGACUCAUAUUCUUAAAAGCCUGGCUCUUGACCCUAUU |
| | GGAAACACAAAGGAAGCUGAAAUCAAACAUCUAAAAUACACUGCGUACAC |
| | GUGUGCGUGCACACACACACACACACACACACACACACAGCUCUUCAUUUC |
| | UCCUGAGCCAUGCAGAAUUUACUUUCAAUGUGGAAAUCUGUUCCCUUUAC |
| | CACACUGUAUAUGCACAGAGCACAAGAGAGGCUAUCUCUAGUCACUUCCA |
| | CCAGCGAGGCCUUAGACUCCGUAUUAGAGGCCACCGAUUUCAUACAACAG |
| | UGUUUCGCUAAAGACCCUUCACUAUUCUUGUUUAGUAAAUAGCUGUCUGC |
| | UCUUCAGGGAACUGUUACCUAUGGGUUAUUACCAAAGAACGCUGGCAAUU |
| | GGAAAUGUCCUGAUGGAAAUUCUUUGCACGUGCCGGUUCUCUGGCAUCCU |
| | CCAGGUGGCCCAACCCAAAGCAGAAAGCAGAAACCACAGACCCCGUGAGU |
| | CUCCCCAUACCUUGUUUCCAAUAACUUGGCAAAACUUCUUGGUGCAUAUU |
| | GGUUACACCCUCUGGGAUUCAUAAUGCCAUUAGGCUAAAACCCUAAGAGA |
| | GAGGGUUGACAGAAACACACGCGAGAAUGAGGCAGAUCCCAGAGCAAGGA |
| | CUGGGCCCAGACUCUCCACAUGUGCUCUACUAGUGAGUGCCUUAUACUCU |
| | CAGUAUUUUGGGGCUUACAGCUUCUUAUUUGUGCUAAAAAGGUGCAGUUC |
| | CAAAGUAGGAACUGCCACACAGGCCCCAGCAUCCUCUCUCCAACUUCAUAC |
| | CUCUCUCCUGGUGGGGGGAGCGGGCAUCCAGGACCUCCGGAAUCAAGGAU |
| | GUGCAGAGAAGAGCGAAAGUAAUUUUUCUAGUCACAUGAACUGAUUGGUU |
| | CCAGGCAAUUAGAAAAUGGCUAUAAAAUAACCUUAAUUUUAAAAAAAAAU |
| | CUUGGGUCUUCGUUUUCCUAUUAGGAGACUGAACUGACCACAUGUAUUGA |
| | UUUAUAUCCUGAAUAUAUGGGAACUUCUGUGUUUGGGAUGUCCUACUGUA |
| | AGACUGAUGAAUGUACAGAGUUAAUUUCAGGGUACAGUUUUGCCUUAAUG |
| | GUUUUAAAAAAUAAACUAUUUUUUAAAAUUU (SEQ ID NO: 311) |
| PPARg | GCCUUAACCUCUGCUGGUGACCAGAAGCCUGCAUUUCUGCAUUCUGCUUA |
| NM_ | AUUCCCUUUCCUUAGAUUUGAAAGAAGCCAACACUAAACCACAAAUAUAC |
| 001330615 | AACAAGGCCAUUUUCUCAAACGAGAGUCAGCCUUUAACGAAAUGACCAUG |
| | GUUGACACAGAGAUGCCAUUCUGGCCCACCAACUUUGGGAUCAGCUCCGU |
| 5468 | GGAUCUCUCCGUAAUGGAAGACCACUCCCACUCCUUUGAUAUCAAGCCCU |
| | UCACUACUGUUGACUUCUCCAGCAUUUCUACUCCACAUUACGAAGACAUU |
| | CCAUUCACAAGAACAGAUCCAGUGGUUGCAGAUUACAAGUAUGACCUGAA |
| | ACUUCAAGAGUACCAAAGUGCAAUCAAAGUGGAGCCUGCAUCUCCACCUU |
| | AUUAUUCUGAGAAGACUCAGCUCUACAAUAAGCCUCAUGAAGAGCCUUCC |
| | AACUCCCUCAUGGCAAUUGAAUGUCGUGUCUGUGGAGAUAAAGCUUCUGG |
| | AUUUCACUAUGGAGUUCAUGCUUGUGAAGGAUGCAAGGGUUUCUUCCGGA |
| | GAACAAUCAGAUUGAAGCUUAUCUAUGACAGAUGUGAUCUUAACUGUCGG |
| | AUCCACAAAAAAAGUAGAAAUAAAUGUCAGUACUGUCGGUUUCAGAAAUG |
| | CCUUGCAGUGGGGAUGUCUCAUAAUGCCAUCAGGUUUGGGCGGAUGCCAC |
| | AGGCCGAGAAGGAGAAGCUGUUGGCGGAGAUCUCCAGUGAUAUCGACCAG |
| | CUGAAUCCAGAGUCCGCUGACCUCCGGGCCCUGGCAAAACAUUUGUAUGA |
| | CUCAUACAUAAAGUCCUUCCCGCUGACCAAAGCAAAGGCGAGGGCGAUCU |
| | UGACAGGAAAGACAACAGACAAAUCAACCGCCCAGGUUUGCUGAAUGUGA |
| | AGCCCAUUGAAGACAUUCAAGACAACCUGCUACAAGCCCUGGAGCUCCAG |
| | CUGAAGCUGAACCACCCUGAGUCCUCACAGCUGUUUGCCAAGCUGCUCCA |
| | GAAAAUGACAGACCUCAGACAGAUUGUCACGGAACACGUGCAGCUACUGC |
| | AGGUGAUCAAGAAGACGGAGACAGACAUGAGUCUUCACCCGCUCCUGCAG |
| | GAGAUCUACAAGGACUUGUACUAGCAGAGAGUCCUGAGCCACUGCCAACA |
| | UUUCCCUUCUUCCAGUUGCACUAUUCUGAGGGAAAAUCUGACACCUAAGA |
| | AAUUUACUGUGAAAAAGCAUUUUAAAAAGAAAAGGUUUUAGAAUAUGAU |
| | CUAUUUUAUGCAUAUUGUUUAUAAAGACACAUUUACAAUUUACUUUUAAU |
| | AUUAAAAAUUACCAUAUUAUGAAA (SEQ ID NO: 312) |
| KLF4 | GGCAGUUUCCCGACCAGAGAGAACGAACGUGUCUGCGGGCGCGCGGGGAG |
| NM_ | CAGAGGCGGUGGCGGGCGGCGGCGGCACCGGGAGCCGCCGAGUGACCCUC |
| 001314052 | CCCCGCCCCUCUGGCCCCCCACCCUCCCACCCGCCCGUGGCCCGCGCCCAUG |
| 9314 | GCCGCGCGCGCUCCACACAACUCACCGGAGUCCGCGCCUUGCGCCGCCGAC |
| | CAGUUCGCAGCUCCGCGCCACGGCAGCCAGUCUCACCUGGCGGCACCGCCC |
| | GCCCACCGCCCCGGCCACAGCCCCUGCGCCCACGGCAGCACUCGAGGCGAC |
| | CGCGACAGUGGUGGGGGACGCUGCUGAGUGGAAGAGAGCGCAGCCCGGCC |
| | ACCGGACCUACUUACUCGCCUUGCUGAUUGUCUAUUUUUGCGUUUACAAC |
| | UUUUCUAAGAACUUUUGUAUACAAAGGAACUUUUUAAAAAAGACGCUUCC |
| | AAGUUAUAUUUAAUCCAAAGAAGAAGGAUCUCGGCCAAUUUGGGGUUUUG |
| | GGUUUUGGCUUCGUUUCUUCUCUUCGUUGACUUUGGGGUUCAGGUGCCCC |
| | AGCUGCUUCGGGCUGCCGAGGACCUUCUGGGCCCCCACAUUAAUGAGGCA |
| | GCCACCUGGCGAGUCUGACAUGGCUGUCAGCGACGCGCUGCUCCCAUCUU |
| | UCUCCACGUUCGCGUCUGGCCCGGCGGGAAGGGAGAAGACACUGCGUCAA |
| | GCAGGUGCCCCGAAUAACCGCUGGCGGGAGGAGCUCUCCCACAUGAAGCG |
| | ACUUCCCCCAGUGCUUCCCGGCCGCCCCUAUGACCUGGCGGCGGCGACCGU |
| | GGCCACAGACCUGGAGAGCGGCGGAGCCGGUGCGGCUUGCGGCGGUAGCA |
| | ACCUGGCGCCCCUACCUCGGAGAGAGACCGAGGAGUUCAACGAUCUCCUG |
| | GACCUGGACUUUAUUCUCUCCAAUUCGCUGACCCAUCCUCCGGAGUCAGU |
| | GGCCGCCACCGUGUCCUCGUCAGCGUCAGCCUCCUCUUCGUCGUCGCCGUC |
| | GAGCAGCGGCCCUGCCAGCGCGCCCUCCACCUGCAGCUUCACCUAUCCGAU |
| | CCGGGCCGGGAACGACCCGGGCGUGGCGCCGGGCGGCACGGGCGGAGGCC |
| | UCCUCUAUGGCAGGGAGUCCGCUCCCCCUCCGACGGCUCCCUUCAACCUGG |
| | CGGACAUCAACGACGUGAGCCCCUCGGGCGGCUUCGUGGCCGAGCUCCUG |
| | CGGCCAGAAUUGGACCCGGUGUACAUUCCGCCGCAGCAGCCGCAGCCGCCA |
| | GGUGGCGGGCUGAUGGGCAAGUUCGUGCUGAAGGCGUCGCUGAGCGCCCC |
| | UGGCAGCGAGUACGGCAGCCCGUCGGUCAUCAGCGUCAGCAAAGGCAGCC |
| | CUGACGGCAGCCACCCGGUGGUGGUGGCGCCCUACAACGGCGGGCCGCCGC |
| | GCACGUGCCCCAAGAUCAAGCAGGAGGCGGUCUCUUCGUGCACCCACUUG |
| | GGCGCUGGACCCCCUCUCAGCAAUGGCCACCGGCCGGCUGCACACGACUUC |
| | CCCCUGGGGCGGCAGCUCCCCAGCAGGACUACCCCGACCCUGGGUCUUGAG |
| | GAAGUGCUGAGCAGCAGGGACUGUCACCCUGCCCUGCCGCUUCCUCCCGGC |
| | UUCCAUCCCCACCCGGGGCCCAAUUACCCAUCCUUCCUGCCCGAUCAGAUG |
| | CAGCCGCAAGUCCCGCCGCUCCAUUACCAAGGUCAGUCCCGGGGAUUUGU |
| | AGCUCGGGCUGGGGAGCCCUGUGUGUGCUGGCCCCACUUCGGGACACACG |
| | GGAUGAUGCUCACCCCACCUUCUUCACCCCUAGAGCUCAUGCCACCCGGUU |
| | CCUGCAUGCCAGAGGAGCCCAAGCCAAAGAGGGGAAGACGAUCGUGGCCC |
| | CGGAAAAGGACCGCCACCCACACUUGUGAUUACGCGGGCUGCGGCAAAAC |
| | CUACACAAAGAGUUCCCAUCUCAAGGCACACCUGCGAACCCACACAGGUG |
| | AGAAACCUUACCACUGUGACUGGGACGGCUGUGGAUGGAAAUUCGCCCGC |
| | UCAGAUGAACUGACCAGGCACUACCGUAAACACACGGGGCACCGCCCGUU |
| | CCAGUGCCAAAAAUGCGACCGAGCAUUUUCCAGGUCGGACCACCUCGCCU |
| | UACACAUGAAGAGGCAUUUUUAAAUCCCAGACAGUGGAUAUGACCCACAC |
| | UGCCAGAAGAGAAUUCAGUAUUUUUUACUUUUCACACUGUCUUCCCGAUG |
| | AGGGAAGGAGCCCAGCCAGAAAGCACUACAAUCAUGGUCAAGUUCCCAAC |
| | UGAGUCAUCUUGUGAGUGGAUAAUCAGGAAAAAUGAGGAAUCCAAAAGAC |
| | AAAAAUCAAAGAACAGAUGGGGUCUGUGACUGGAUCUUCUAUCAUUCCAA |
| | UUCUAAAUCCGACUUGAAUAUUCCUGGACUUACAAAAUGCCAAGGGGGUG |
| | ACUGGAAGUUGUGGAUAUCAGGGUAUAAAUUAUAUCCGUGAGUUGGGGG |
| | AGGGAAGACCAGAAUUCCCUUGAAUUGUGUAUUGAUGCAAUAUAAGCAUA |
| | AAAGAUCACCUUGUAUUCUCUUUACCUUCUAAAAGCCAUUAUUAUGAUGU |
| | UAGAAGAAGAGGAAGAAAUUCAGGUACAGAAAACAUGUUUAAAUAGCCU |
| | AAAUGAUGGUGCUUGGUGAGUCUUGGUUCUAAAGGUACCAAACAAGGAAG |
| | CCAAAGUUUUCAAACUGCUGCAUACUUUGACAAGGAAAAUCUAUAUUUGU |
| | CUUCCGAUCAACAUUUAUGACCUAAGUCAGGUAAUAUACCUGGUUUACUU |
| | CUUUAGCAUUUUUAUGCAGACAGUCUGUUAUGCACUGUGGUUUCAGAUGU |
| | GCAAUAAUUUGUACAAUGGUUUAUUCCCAAGUAUGCCUUAAGCAGAACAA |
| | AUGUGUUUUUCUAUAUAGUUCCUUGCCUUAAUAAAUAUGUAAUAUAAAU |
| | UUAAGCAAACGUCUAUUUUGUAUAUUUGUAAACUACAAAGUAAAAUGAAC |
| | AUUUUGUGGAGUUUGUAUUUUGCAUACUCAAGGUGAGAAUUAAGUUUUA |
| | AAUAAACCUAUAAUAUUUUAUCUGAA (SEQ ID NO: 313) |
| KLF6 | GCAGUCAGUCCGGUGUUUGCAAAUAUUGUGUGGGCUCCGCGCGCUGCGGG |
| NM_ | CUGCGGGAGGGUCCGGCCGGGCGUCUCUGCGAGCCUGGAGUUUGCAUGAA |
| 001160124 | ACUUUCACCUGCGCUCCGGGGAGACUUUCGGCUCCGGCUCCCACCGCGCGC |
| 1316 | CUCGCCGCCCUCGCGACCGCGGGCUCCGUCCAACCCGGCCCGACAUGGACG |
| | UGCUCCCCAUGUGCAGCAUCUUCCAGGAGCUCCAGAUCGUGCACGAGACC |
| | GGCUACUUCUCGGCGCUGCCGUCUCUGGAGGAGUACUGGCAACAGACCUG |
| | CCUAGAGCUGGAACGUUACCUCCAGAGCGAGCCCUGCUAUGUUUCAGCCU |
| | CAGAAAUCAAAUUUGACAGCCAGGAAGAUCUGUGGACCAAAAUCAUUCUG |
| | GCUCGGGAGAAAAAGGAGGAAUCCGAACUGAAGAUAUCUUCCAGUCCUCC |
| | AGAGGACACUCUCAUCAGCCCGAGCUUUUGUUACAACUUAGAGACCAACA |
| | GCCUGAACUCAGAUGUCAGCAGCGAAUCCUCUGACAGCUCCGAGGAACUU |
| | UCUCCCACGGCCAAGUUUACCUCCGACCCCAUUGGCGAAGUUUUGGUCAG |
| | CUCGGGAAAAUUGAGCUCCUCUGUCACCUCCACGCCUCCAUCUUCUCCGGA |
| | ACUGAGCAGGGAACCUUCUCAACUGUGGGGUUGCGUGCCCGGGGAGCUGC |
| | CCUCGCCAGGGAAGGUGCGCAGCGGGACUUCGGGGAAGCCAGGAGAAAAG |
| | CCUUACAGAUGCUCAUGGGAAGGGUGUGAGUGGCGUUUUGCAAGAAGUGA |
| | UGAGUUAACCAGGCACUUCCGAAAGCACACCGGGGCCAAGCCUUUUAAAU |
| | GCUCCCACUGUGACAGGUGUUUUUCCAGGUCUGACCACCUGGCCCUGCAC |
| | AUGAAGAGGCACCUCUGAGGGAGCAGAGAGGUGGAUCCUGUAGGCUAAAA |
| | GGCUUCCAGGCUGAGAGCCGGCCGUGGAAGGAGGGAUGCGUGUUCCAGCC |
| | AAAGCAUGCCGUUCUGCACCCUACCCAGUUGCCUCCAGGGCCUCUCCUUGG |
| | AAGGUCUUUUGAGGGCUAAAAAGGUCCUGUAAGAAGCGGCAUAGCACCCG |
| | UGGUGCAUGGUAUGUGGGUGACCCUGGACUCGCCACUGGUACCCGCCCUU |
| | CCGAGCGGCGCCUAAGCCUUUGCCGUGAGCAUGCACACUGAGAAUGCUAA |
| | UGGUUGGGUUGAUUGUAUGUUGAGGAUCUAUUACUGACCGUAUGAUGAG |
| | GCCAACUUUUUUUCCUUGUGGUUAGCAAGACUGCAAGAGAUGGAAAAAAA |
| | GUAGUUUGAAUGUUUUGUGUGUAAGGAGUAUACCAUGAGAUGAGAUGAC |
| | CACCAAUCAUUUCCUUGGGGGGAGGGGGUGUCUGCACCUUAGAAAAAAAA |
| | AGAAAAAUCAAAAAAACAAAAAAACAAAAACAAAAAAAGAAGGAAAAUC |
| | UUGGAGGGUGGGCGUGGGAACUCAGGACCCCAGAGUGGCGAGUGGUGUGG |
| | GGAGGGAGAGCCUCUCUCCCCCUUUUCUGUGUGAGAGGAACUCUUAGUGU |
| | CUGGUGCAGCUAUUAAAUGUGCAAUGUGUCAAGUAGCUUGUUUUACACGC |
| | UACAACAUAGCUCAUUUGUAACCCAUUGUAUAAGCUGUGUAUUUACAAAU |
| | AUAACACAACAAUUUAACUUUUCCUUAGAAUACAAAAAGUCAUGCAUGGU |
| | CUGGGGAACUAUAUGCUUUUCCAUUUUUAAGUCAGGACUGCAAUACUGAU |
| | UCCAGUUAAUGAGCAGCUAAGAUCCAAUCUGUCUAAUACAGUGACCCCCU |
| | AGCCAUCCGGGCCUGGCAAUAUACAAUUUUUUUUCCCCUCCAAGUUUGUA |
| | ACACUCCCCUUCCAGAAAGGCAUUGUGCAACACAGGAUUAUUUUUAAAUG |
| | AUUCUGAAUUUGAAUUAACUUUUUGGAGAAUUCUGUGAUGCCCUUAGAAG |
| | AAAUUGGACACGUAUUGAGUGUCACAAAGCUGGGGCUGGGAAUUGCUGGU |
| | CUAAUGUUUCAUUAGACUUAAGAACCUAAAAUUUUUCUCAGUUGGGUGGA |
| | UAAAACCACUAACGCUUAGAAACUGUUUUCUCAUGCAGCUAUGUUUCUCU |
| | UAUUUAUGCCUUGAGGACUAAUUUCUGGUUUUCUAGCUGUUAAUGCACUG |
| | UUGACCUUCAUAAUGGUGCCUUACGCAAGCGAUCCCUUCUGUGGGGGUCU |
| | CAUACAGGGGUGUGGGCGAUGCAUGCUUUAUUAAGGCUCUUGUUUCACCU |
| | GGCAGUGUACUGUAUCAACGUAUAAUACAGAAAAAAAAUCUCUUUAAGGU |
| | CCUCCUUCACAAAGACAUAGAGUGAAACUCCCUUUACAUGUCAGUAUUUG |
| | UUCAACACUUUAGGCAACUUGACUGUCAGUGUUAAAAUGGAAAACAGGAA |
| | AAUGGAAAAAUCUGACCAAUUCUGCCACCUUGAGACUUUCAUAUAGACCU |
| | UGCACAACAAUUGUAUAGAUCACACACCGGCUGUAUUUAAUAUGUAACAU |
| | UUUCACACAUAUUAAAGAUACAGAAGUAUUAAAAAACCCCCAAUGUUAAU |
| | GUAUUUGCUUAAAAGGCACAAGUUUCACAUAUCUGUCUAGCUAUCUGUUG |
| | GUAAUACAGAAAGUAUACUACUUUUUUAAAAAAGUGGGCAGAAUUCUUG |
| | UGUAUGUAUAUUUGUGUGUACAGUAUGUGUAUGUGUGUAUAUAUAUAUA |
| | UUAUAUAUAUAGAUAAUAUAUAAAUAUUUUUUUUAAGGAGAAACUAGAA |
| | UGUUUAGCUAGAAAAUUCCACAGCCUGUGAAGAAAUAUUUCAAAAUGGCC |
| | AUAAAGGAGGUAAAAAUGAAAACCAUAACCUAACUUUUAUAGAGGCUUUA |
| | UCUUUAAUUUAACGAUGUGCGGAGGACUUUCUUGCUUGAAUCUGUUCCGG |
| | GCUGUCUGCUCUGUCCAUCAAAUGGGCAGGUCUGGAAUGGGGCACCUUCG |
| | GCCGUUCAGAAGUGGCCUGAACAGAAUGCUGGAACCCAGGCUGGACUCGG |
| | ACACACUAAGGUUUUGAUUUUGAAUUUCAGCCUUAUUAGAAGAUCUAACC |
| | UAAGAGUAAGCUAACCACAGGGAUUCUUUUGUAGAACACUUUUUAUGCAG |
| | AUGAAGCUAUUUUUUCCAGCAAGUAGAUUCUUCCAGUUUUUCCAAGGAGU |
| | AAUUUCCCCGAAUUGGCAUACCACGGCGUGGACAGCUGAUAUUUCACCCA |
| | GCUGCUGGCUUGUGGGUGUGGCUCUUUGCUUUAUAUAUAUAUACACACAU |
| | GUGAGUCUGGCUGGGCUGGUAUUUUGUUUGAUCUUCCUGGAAAUGAGCAG |
| | UGACUAACGCUCACAUAACUGGUUUUUUUUUUAUCUGGGCUGAUGAAUAC |
| | AUUUACCUAAGAAACUCAUUUCGUUUUACUUAAGAGGGGAAGUGCAGUUU |
| | UCUUUUGGCAGUUCAGAAUCCAAGCACUUGAUUUGCUGGGUUUGGAAAAC |
| | UCCUUUUUUGGCCUUCUAUGUGCUUAGCCAUAACAAUUCCAUUAAGCAAG |
| | AAGGUAAGCAAAAGACAAAAAAAAAAAAAGGAAAAAAAAAAACUUGCAC |
| | UGGCUUGUCUCACUUACGAAACAUGUCGGAGCUGUUUGCCUGGGUGGGGC |
| | UGGGUACCGUACCUGUCAAUGCCUGUGAUUUUCAUAAUUAGCACGUACAU |
| | AAAGAAGUACAUUCUGUUCAGGUGAUAACUGAGCCUCAAUCAAGCAGAAA |
| | CUUUUUGCUUGAAAUUAAAAAAAAAUUUCUAUUAGUGAAAUUUCUUUUU |
| | UUUUUUUUUUUGGAAGCACCCUGUUAUCUAAAGAAUCUUUGUAAGAUUUU |
| | UGUAAAAUUUUGUUUUACAAGAUUUUAUUUGAAAUUGUUUUUUGCAAGA |
| | UUGUUAUAUUUCUGUAUGAAUGUAUUUUUUAUUGGAAUAACAUAAAAGA |
| | AUUCUUAUCAGCAUCUUGAGUCUGGUUGUUUUUUUUGGGGGAAGGGGGU |
| | UGUUGGAACAGAUUCCUCCUGCAUUCAUCACCCUGGCUUCCUCCUGGGGG |
| | CAAAUCUUCAUUGAGCAACCCUGAGAACAACUCACCGACCUCAGCCCCUUC |
| | CUCUUUCCACAGCCUGUCCUGGGAGCUGGAGAGGAUGUCAGCGAGCCUGA |
| | CAUUGCCCUCCCUGAAUGCAUCAAAUACUCUUCUCCAAGGACUGACAAAA |
| | ACAACCCCCCGGCUGUGGGCACAGUAGGGAACCAUGCCGGUUUGAUCUUC |
| | CAUUGCUCAAGCCAGGGAAUGCAUUGCAGAGGCACCUGCACAACGACAAA |
| | CUUGAGUGUUUCCCUGCCACUUCUGUGCCUCCACAGCCUGCUCCAGUUCCG |
| | CAGCUAGAGCUGGGCCUACCUUCCCCAGCCAGCCCUGCCACACACCUGGCU |
| | GAGGCAUGUCUGGGAGGGGUGGAGGAGGCAGAGGGACUCCAGGCCAGAGG |
| | GCUGUUCUCACAACCUCAGCCCACUCUCAGUCAAGGAGGGCAGAAAAUAA |
| | AAGAUGACAUCACUGCCA (SEQ ID NO: 314) |
| C/EBP- | AUUCGCGACCCGGAGGUGCGCGGGCGCGGGCGAGCAGGGUCUCCGGGUGG |
| alpha | GCGGCGGCGACGCCCCGCGCAGGCUGGAGGCCGCCGAGGCUCGCCAUGCCG |
| NM_ | GGAGAACUCUAACUCCCCCAUGGAGUCGGCCGACUUCUACGAGGCGGAGC |
| 001285829 | CGCGGCCCCCGAUGAGCAGCCACCUGCAGAGCCCCCCGCACGCGCCCAGCA |
| 1050 | GCGCCGCCUUCGGCUUUCCCCGGGGCGCGGGCCCCGCGCAGCCUCCCGCCC |
| | CACCUGCCGCCCCGGAGCCGCUGGGCGGCAUCUGCGAGCACGAGACGUCCA |
| | UCGACAUCAGCGCCUACAUCGACCCGGCCGCCUUCAACGACGAGUUCCUGG |
| | CCGACCUGUUCCAGCACAGCCGGCAGCAGGAGAAGGCCAAGGCGGCCGUG |
| | GGCCCCACGGGCGGCGGCGGCGGCGGCGACUUUGACUACCCGGGCGCGCCC |
| | GCGGGCCCCGGCGGCGCCGUCAUGCCCGGGGGAGCGCACGGGCCCCCGCCC |
| | GGCUACGGCUGCGCGGCCGCCGGCUACCUGGACGGCAGGCUGGAGCCCCU |
| | GUACGAGCGCGUCGGGGCGCCGGCGCUGCGGCCGCUGGUGAUCAAGCAGG |
| | AGCCCCGCGAGGAGGAUGAAGCCAAGCAGCUGGCGCUGGCCGGCCUCUUC |
| | CCUUACCAGCCGCCGCCGCCGCCGCCGCCCUCGCACCCGCACCCGCACCCGC |
| | CGCCCGCGCACCUGGCCGCCCCGCACCUGCAGUUCCAGAUCGCGCACUGCG |
| | GCCAGACCACCAUGCACCUGCAGCCCGGUCACCCCACGCCGCCGCCCACGC |
| | CCGUGCCCAGCCCGCACCCCGCGCCCGCGCUCGGUGCCGCCGGCCUGCCGG |
| | GCCCUGGCAGCGCGCUCAAGGGGCUGGGCGCCGCGCACCCCGACCUCCGCG |
| | CGAGUGGCGGCAGCGGCGCGGGCAAGGCCAAGAAGUCGGUGGACAAGAAC |
| | AGCAACGAGUACCGGGUGCGGCGCGAGCGCAACAACAUCGCGGUGCGCAA |
| | GAGCCGCGACAAGGCCAAGCAGCGCAACGUGGAGACGCAGCAGAAGGUGC |
| | UGGAGCUGACCAGUGACAAUGACCGCCUGCGCAAGCGGGUGGAACAGCUG |
| | AGCCGCGAACUGGACACGCUGCGGGGCAUCUUCCGCCAGCUGCCAGAGAG |
| | CUCCUUGGUCAAGGCCAUGGGCAACUGCGCGUGAGGCGCGCGGCUGUGGG |
| | ACCGCCCUGGGCCAGCCUCCGGCGGGGACCCAGGGAGUGGUUUGGGGUCG |
| | CCGGAUCUCGAGGCUUGCCCGAGCCGUGCGAGCCAGGACUAGGAGAUUCC |
| | GGUGCCUCCUGAAAGCCUGGCCUGCUCCGCGUGUCCCCUCCCUUCCUCUGC |
| | GCCGGACUUGGUGCGUCUAAGAUGAGGGGGCCAGGCGGUGGCUUCUCCCU |
| | GCGAGGAGGGGAGAAUUCUUGGGGCUGAGCUGGGAGCCCGGCAACUCUAG |
| | UAUUUAGGAUAACCUUGUGCCUUGGAAAUGCAAACUCACCGCUCCAAUGC |
| | CUACUGAGUAGGGGGAGCAAAUCGUGCCUUGUCAUUUUAUUUGGAGGUUU |
| | CCUGCCUCCUUCCCGAGGCUACAGCAGACCCCCAUGAGAGAAGGAGGGGA |
| | GCAGGCCCGUGGCAGGAGGAGGGCUCAGGGAGCUGAGAUCCCGACAAGCC |
| | CGCCAGCCCCAGCCGCUCCUCCACGCCUGUCCUUAGAAAGGGGUGGAAACA |
| | UAGGGACUUGGGGCUUGGAACCUAAGGUUGUUCCCCUAGUUCUACAUGAA |
| | GGUGGAGGGUCUCUAGUUCCACGCCUCUCCCACCUCCCUCCGCACACACCC |
| | CACCCCAGCCUGCUAUAGGCUGGGCUUCCCCUUGGGGCGGAACUCACUGC |
| | GAUGGGGGUCACCAGGUGACCAGUGGGAGCCCCCACCCCGAGUCACACCA |
| | GAAAGCUAGGUCGUGGGUCAGCUCUGAGGAUGUAUACCCCUGGUGGGAGA |
| | GGGAGACCUAGAGAUCUGGCUGUGGGGGGGGCAUGGGGGGUGAAGGGCCA |
| | CUGGGACCCUCAGCCUUGUUUGUACUGUAUGCCUUCAGCAUUGCCUAGGA |
| | ACACGAAGCACGAUCAGUCCAUCCCAGAGGGACCGGAGUUAUGACAAGCU |
| | UUCCAAAUAUUUUGCUUUAUCAGCCGAUAUCAACACUUGUAUCUGGCCUC |
| | UGUGCCCCAGCAGUGCCUUGUGCAAUGUGAAUGUGCGCGUCUCUGCUAAA |
| | CCACCAUUUUAUUUGGUUUUUGUUUUGUUUUGGUUUUGCUCGGAUACUUG |
| | CCAAAAUGAGACUCUCCGUCGGCAGCUGGGGGAAGGGUCUGAGACUCCCU |
| | UUCCUUUUGGUUUUGGGAUUACUUUUGAUCCUGGGGGACCAAUGAGGUGA |
| | GGGGGGUUCUCCUUUGCCCUCAGCUUUCCCCAGCCCCUCCGGCCUGGGCUG |
| | CCCACAAGGCUUGUCCCCCAGAGGCCCUGGCUCCUGGUCGGGAAGGGAGG |
| | UGGCCUCCCGCCAACGCAUCACUGGGGCUGGGAGCAGGGAAGGACGGCUU |
| | GGUUCUCUUCUUUUGGGGAGAACGUAGAGUCUCACUCUAGAUGUUUUAUG |
| | UAUUAUAUCUAUAAUAUAAACAUAUCAAAGUCAA (SEQ ID NO: 315) |
| C/EBP-beta | UCCCAAUCCCGGGGCGGCCGGGCGGGGGUGGGCAGGGGGCGUGAGGCCGC |
| NM_ | CCCUGCGUCCCGGGGGCCCCCCGAAAACGCGCUCCGGGUGCCCGGUCCCUC |
| 001285878 | CGCUGCGCCCUGCCGCCGUCCUCCCGGGGGUCUCGGGCGGCCGCGGCCGUG |
| 1051 | UCCUUCGCGUCCCGGCGGCGCGGCGGGAGGGGCCGGCGUGACGCAGCGGU |
| | UGCUACGGGCCGCCCUUAUAAAUAACCGGGCUCAGGAGAAACUUUAGCGA |
| | GUCAGAGCCGCGCACGGGACUGGGAAGGGGACCCACCCGAGGGUCCAGCC |
| | ACCAGCCCCCUCACUAAUAGCGGCCACCCCGGCAGCGGCGGCAGCAGCAGC |
| | AGCGACGCAGCGGCGACAGCUCAGAGCAGGGAGGCCGCGCCACCUGCGGG |
| | CCGGCCGGAGCGGGCAGCCCCAGGCCCCCUCCCCGGGCACCCGCGUUCAUG |
| | CAACGCCUGGUGGCCUGGGACCCAGCAUGUCUCCCCCUGCCGCCGCCGCCG |
| | CCUGCCUUUAAAUCCAUGGAAGUGGCCAACUUCUACUACGAGGCGGACUG |
| | CUUGGCUGCUGCGUACGGCGGCAAGGCGGCCCCCGCGGCGCCCCCCGCGGC |
| | CAGACCCGGGCCGCGCCCCCCCGCCGGCGAGCUGGGCAGCAUCGGCGACCA |
| | CGAGCGCGCCAUCGACUUCAGCCCGUACCUGGAGCCGCUGGGCGCGCCGCA |
| | GGCCCCGGCGCCCGCCACGGCCACGGACACCUUCGAGGCGGCUCCGCCCGC |
| | GCCCGCCCCCGCGCCCGCCUCCUCCGGGCAGCACCACGACUUCCUCUCCGA |
| | CCUCUUCUCCGACGACUACGGGGGCAAGAACUGCAAGAAGCCGGCCGAGU |
| | ACGGCUACGUGAGCCUGGGGCGCCUGGGGGCCGCCAAGGGCGCGCUGCAC |
| | CCCGGCUGCUUCGCGCCCCUGCACCCACCGCCCCCGCCGCCGCCGCCGCCCG |
| | CCGAGCUCAAGGCGGAGCCGGGCUUCGAGCCCGCGGACUGCAAGCGGAAG |
| | GAGGAGGCCGGGGCGCCGGGCGGCGGCGCAGGCAUGGCGGCGGGCUUCCC |
| | GUACGCGCUGCGCGCUUACCUCGGCUACCAGGCGGUGCCGAGCGGCAGCA |
| | GCGGGAGCCUCUCCACGUCCUCCUCGUCCAGCCCGCCCGGCACGCCGAGCC |
| | CCGCUGACGCCAAGGCGCCCCCGACCGCCUGCUACGCGGGGGCCGCGCCGG |
| | CGCCCUCGCAGGUCAAGAGCAAGGCCAAGAAGACCGUGGACAAGCACAGC |
| | GACGAGUACAAGAUCCGGCGCGAGCGCAACAACAUCGCCGUGCGCAAGAG |
| | CCGCGACAAGGCCAAGAUGCGCAACCUGGAGACGCAGCACAAGGUCCUGG |
| | AGCUCACGGCCGAGAACGAGCGGCUGCAGAAGAAGGUGGAGCAGCUGUCG |
| | CGCGAGCUCAGCACCCUGCGGAACUUGUUCAAGCAGCUGCCCGAGCCCCUG |
| | CUCGCCUCCUCCGGCCACUGCUAGCGCGGCCCCCGCGCGCGUCCCCCUGCC |
| | GGCCGGGGCUGAGACUCCGGGGAGCGCCCGCGCCCGCGCCCUCGCCCCCGC |
| | CCCCGGCGGCGCCGGCAAAACUUUGGCACUGGGGCACUUGGCAGCGCGGG |
| | GAGCCCGUCGGUAAUUUUAAUAUUUUAUUAUAUAUAUAUAUCUAUAUUU |
| | UUGUCCAAACCAACCGCACAUGCAGAUGGGGCUCCCGCCCGUGGUGUUAU |
| | UUAAAGAAGAAACGUCUAUGUGUACAGAUGAAUGAUAAACUCUCUGCUUC |
| | UCCCUCUGCCCCUCUCCAGGCGCCGGCGGGCGGGCCGGUUUCGAAGUUGA |
| | UGCAAUCGGUUUAAACAUGGCUGAACGCGUGUGUACACGGGACUGACGCA |
| | ACCCACGUGUAACUGUCAGCCGGGCCCUGAGUAAUCGCUUAAAGAUGUUC |
| | CUACGGGCUUGUUGCUGUUGAUGUUUUGUUUUGUUUUGUUUUUUGGUCU |
| | UUUUUUGUAUUAUAAAAAAUAAUCUAUUUCUAUGAGAAAAGAGGCGUCU |
| | GUAUAUUUUGGGAAUCUUUUCCGUUUCAAGCAUUAAGAACACUUUUAAUA |
| | AACUUUUUUUUGAGAAUGGUUACAAAGCCUUUUGGGGGCAGUAAAAAAA |
| | (SEQ ID NO: 316) |
| TOX | CUCUUCUUCUUAAACAAACCACAAACGGAUGUGAGGGAAGGAAGGUGUUU |
| NM_ | CUUUUACUCCUGAGCCCAGACACCUCACUCUGUUCCGUCUAAGCUUGUUU |
| 014729 | UGCUGAACACUUUUUUUUAAAAAAGGAAAAAGAAAAGGAGUUGCUUGAU |
| 9760 | GUGAGAGUGAAAUGGACGUAAGAUUUUAUCCACCUCCAGCCCAGCCCGCC |
| | GCUGCGCCCGACGCUCCCUGUCUGGGACCUUCUCCCUGCCUGGACCCCUAC |
| | UAUUGCAACAAGUUUGACGGUGAGAACAUGUAUAUGAGCAUGACAGAGCC |
| | GAGCCAGGACUAUGUGCCAGCCAGCCAGUCCUACCCUGGUCCAAGCCUGG |
| | AAAGUGAAGACUUCAACAUUCCACCAAUUACUCCUCCUUCCCUCCCAGACC |
| | ACUCGCUGGUGCACCUGAAUGAAGUUGAGUCUGGUUACCAUUCUCUGUGU |
| | CACCCCAUGAACCAUAAUGGCCUGCUACCAUUUCAUCCACAAAACAUGGA |
| | CCUCCCUGAAAUCACAGUCUCCAAUAUGCUGGGCCAGGAUGGAACACUGC |
| | UUUCUAAUUCCAUUUCUGUGAUGCCAGAUAUACGAAACCCAGAAGGAACU |
| | CAGUACAGUUCCCAUCCUCAGAUGGCAGCCAUGAGACCAAGGGGCCAGCC |
| | UGCAGACAUCAGGCAGCAGCCAGGAAUGAUGCCACAUGGCCAGCUGACUA |
| | CCAUUAACCAGUCACAGCUAAGUGCUCAACUUGGUUUGAAUAUGGGAGGA |
| | AGCAAUGUUCCCCACAACUCACCAUCUCCACCUGGAAGCAAGUCUGCAAC |
| | UCCUUCACCAUCCAGUUCAGUGCAUGAAGAUGAAGGCGAUGAUACCUCUA |
| | AGAUCAAUGGUGGAGAGAAGCGGCCUGCCUCUGAUAUGGGGAAAAAACCA |
| | AAAACUCCCAAAAAGAAGAAGAAGAAGGAUCCCAAUGAGCCCCAGAAGCC |
| | UGUGUCUGCCUAUGCGUUAUUCUUUCGUGAUACUCAGGCCGCCAUCAAGG |
| | GCCAAAAUCCAAACGCUACCUUUGGCGAAGUCUCUAAAAUUGUGGCUUCA |
| | AUGUGGGACGGUUUAGGAGAAGAGCAAAAACAGGUCUAUAAAAAGAAAA |
| | CCGAGGCUGCGAAGAAGGAGUACCUGAAGCAACUCGCAGCAUACAGAGCC |
| | AGCCUUGUAUCCAAGAGCUACAGUGAACCUGUUGACGUGAAGACAUCUCA |
| | ACCUCCUCAGCUGAUCAAUUCGAAGCCGUCGGUGUUCCAUGGGCCCAGCC |
| | AGGCCCACUCGGCCCUGUACCUAAGUUCCCACUAUCACCAACAACCGGGAA |
| | UGAAUCCUCACCUAACUGCCAUGCAUCCUAGUCUCCCCAGGAACAUAGCCC |
| | CCAAGCCGAAUAACCAAAUGCCAGUGACUGUCUCUAUAGCAAACAUGGCU |
| | GUGUCCCCUCCUCCUCCCCUCCAGAUCAGCCCGCCUCUUCACCAGCAUCUC |
| | AACAUGCAGCAGCACCAGCCGCUCACCAUGCAGCAGCCCCUUGGGAACCAG |
| | CUCCCCAUGCAGGUCCAGUCUGCCUUACACUCACCCACCAUGCAGCAAGGA |
| | UUUACUCUUCAACCCGACUAUCAGACUAUUAUCAAUCCUACAUCUACAGC |
| | UGCACAAGUUGUCACCCAGGCAAUGGAGUAUGUGCGUUCGGGGUGCAGAA |
| | AUCCUCCCCCACAACCGGUGGACUGGAAUAACGACUACUGCAGUAGUGGG |
| | GGCAUGCAGAGGGACAAAGCACUGUACCUUACUUGAGAAUCUGAACACCU |
| | CUUCUUUCCACUGAGGAAUUCAGGGAAGUGUUUUCACCAUGGAUUGCUUU |
| | GUACAGUCAAGGCAGUUCUCCAUUUUAUUAGAAAAUACAAGUUGCUAAGC |
| | ACUUAGGACCAUUUGAGCUUGUGGGUCACCCACUCUGGAAGAAAUAGUCA |
| | UGCUUCUUUAUUAUUUUUUUAAUCCUUUAUGGACAUUGUUUUUCUUCUUC |
| | CCUGAAGGAAAUUUGGACCAUUCAGAUUUUAUGUUGGUUUUUUGCUGUGA |
| | AGUGCUGCGCUCUAGUAACUGCCUUAGCAACUGUAGAUGUCUCGGAUAAA |
| | AGUCCUGGAUUUUCCAUUGGUUUUCAUAAUGGGUGUUUAUAUGAAACUAC |
| | UAAAGACUUUUUAAAUGGCUUGAUGUAGCAGUCAUAGCAAGUUUGUAAA |
| | UAGCAUCUAUGUUACACUCUCCUAGAGUAUAAAAUGUGAAUGUUUUUGUA |
| | GCUAAAUUGUAAUUGAAACUGGCUCAUUCCAGUUUAUUGAUUUCACAAUA |
| | GGGGUUAAAUUGGCAAACAUUCAUAUUUUUACUUCAUUUUUAAAACAACU |
| | GACUGAUAGUUCUAUAUUUUCAAAAUAUUUGAAAAUAAAAAGUAUUCCCA |
| | AGUGAUUUUAAUUUAAAAACAAAUUGGCUUUGUCUCAUUGAUCAGACAAA |
| | AAGAAACUAGUAUUAAGGGAAGCGCAAACACAUUUAUUUUGUACUGCAGA |
| | AAAAUUGCUUUUUUGUAUCACUUUUUGUGUAAUGGUUAGUAAAUGUCAU |
| | UUAAGUCCUUUUAUGUAUAAAACUGCCAAAUGCUUACCUGGUAUUUUAUU |
| | AGAUGCAGAAACAGAUUGGAAACAGCUAAAUUACAACUUUUACAUAUGGC |
| | UCUGUCUUAUUGUUUCUUCAUACUGUGUCUGUAUUUAAUCUUUUUUUAUG |
| | GAACCUGUUGCGCCUAUUUAUGAAAUAAUAAAUAUAGGUGUUUGUAAGU |
| | AAAUUUGUUAGUAUUUGAAAGAGGUUUCUUUGAUGUUUUAACUUUUGCU |
| | GGCAAAAAAAAAUUCACGCUUGGUGUGAAUACUUUAUUAUUUAGUUUUU |
| | ACAGUAACAUGAAUAAAGCCAAACCUGCUUUUCAUUUAGCAGCAAAUUAA |
| | AGUAACCAGUCCUUAUUUCUGCAUUUCUUUGGUUGAUGCAAACAAAAAAC |
| | UAUUAUAUUUAAGAACUUUAUUUCUUCAUACGACAUAACAGAAUUGCCCU |
| | CCAAGUCACACAAGCUCCAAGACUAAACAAACAGACAGGUCCUCUGUCUU |
| | AAAAAGGUUACUUCUUGGUUCUCAGCUGGUUCUAGUCAAUUCUGAACCAC |
| | CACCCCCCGCCCCCCGCAAAAAAGUAAAAGUCAAACCAAACUUCCUCAAGC |
| | UGCAUGCUUUUCACAAAAUCCAGAAAGCAUUUAAGAAUUGAACUAGGGGC |
| | UGGAAGAAGUGAAAGGGAAGCAUCUAAAAAUGAAAGGUGAGUAACCAGA |
| | UAGCAAAAGAAAAGGGAAAGCCAUCCAAAUUUGAAAGCUGUUGAUAGAAA |
| | UUGAGAUUCUUGCUGUCUUUUGUGCCUCUACAAGCUACUACUCAUUCCAG |
| | AAUUCCUGGGUCUUCCAAGAGGAUUCUUAAGGUACCAGAGAUUUGCUAGG |
| | GAACCAAAAGUGCUUGAGAAUCUGCCUGAGGGCUUGCAUAGCUUUCACAU |
| | UAAAAAAAGAAAAAGCUAGCAGAUUUACUCCUUUUUAGGGGAUCAUAUCA |
| | AGAAAGUUAGUCUGGUUGGAAACCAAGAGAAUGGCUGAUGUCUCUUUCUU |
| | GGAAUAUGUGAAAUAAAUUUAGCAGUUUAACUAAAUACAAAUAUAUGCA |
| | UUGUGUAAUCCACUCAGAAUUAAACAGACAAAAGGUAUGCUUGCUUUGGA |
| | AUGAUUUUAGGCAUUGUACAACCUUGAAUCACUUGAGCAUGUAAUAACUA |
| | AUAAAUAAUGCAGAUCCAUGUGAUUAUUAAAAUGACUGUAGCUGAGAGCU |
| | CUAAUUUUCCUGUCUUGAAACUGUAUAAGAACUCAUGUGAUUAAGUUCAC |
| | AGUUUAUUGUUUGUCUGUUUAGUAUUUUAGAAAUAUACCAGCACUACUAA |
| | UUAACUAAUGUCUUUUAUUUAUUAUAUUAUGAUAAAGUAAAAAUUUCAC |
| | UUGCAUUAAGUCUAAACUGAGAAGGUAAUUACUGGGAGGAGAAUGAGCA |
| | GCUUUGACUUUGACAGGCGGUUUGUGCAGGAAAGCACAGUGCCGUGUUGU |
| | UUACAGCUUUUCUAGAGCAGCUGUGCGACCAGGGUAGAGAGUGUUGAAAU |
| | UCAAUACCAAAUACAGUAAAAACAAAUGUAAAUAAAAGAAAACACAUCAU |
| | CAAUAAAACUGUUAUUAUGCGUGACCGUA (SEQ ID NO: 317) |
| YTHDF2 | AGAGCGUCGCCGAGUCGGAGCCGGAGCCUGAGCCGCGCGCUGUGUCUCCG |
| NM_ | CUGCGUCCGCCGAGGCCCCCGAGUGUCAGGGACAAAAGCCUCCGCCUGCUC |
| 001172828 | CCGCAGACGGGGCUCAUCUGCCGCCGCCGCCGCGCUGAGGAGAGUUCGCCG |
| 51441 | CCGUCGCCGCCCGUGAGGAUCUGAGAGCCAUGUCGGCCAGCAGCCUCUUG |
| | GAGCAGUACAAAAUGGAUCUGUACAUCAAAAGGAUGGAUUAAACGAUGAU |
| | GAUUUUGAACCUUACUUGAGUCCACAGGCAAGGCCCAAUAAUGCAUAUAC |
| | UGCCAUGUCAGAUUCCUACUUACCCAGUUACUACAGUCCCUCCAUUGGCU |
| | UCUCCUAUUCUUUGGGUGAAGCUGCUUGGUCUACGGGGGGUGACACAGCC |
| | AUGCCCUACUUAACUUCUUAUGGACAGCUGAGCAACGGAGAGCCCCACUU |
| | CCUACCAGAUGCAAUGUUUGGGCAACCAGGAGCCCUAGGUAGCACUCCAU |
| | UUCUUGGUCAGCAUGGUUUUAAUUUCUUUCCCAGUGGGAUUGACUUCUCA |
| | GCAUGGGGAAAUAACAGUUCUCAGGGACAGUCUACUCAGAGCUCUGGAUA |
| | UAGUAGCAAUUAUGCUUAUGCACCUAGCUCCUUAGGUGGAGCCAUGAUUG |
| | AUGGACAGUCAGCUUUUGCCAAUGAGACCCUCAAUAAGGCUCCUGGCAUG |
| | AAUACUAUAGACCAAGGGAUGGCAGCACUGAAGUUGGGUAGCACAGAAGU |
| | UGCAAGCAAUGUUCCAAAAGUUGUAGGUUCUGCUGUUGGUAGCGGGUCCA |
| | UUACUAGUAACAUCGUGGCUUCCAAUAGUUUGCCUCCAGCCACCAUUGCU |
| | CCUCCAAAACCAGCAUCUUGGGCUGAUAUUGCUAGCAAGCCUGCAAAACA |
| | GCAACCUAAACUGAAGACCAAGAAUGGCAUUGCAGGGUCAAGUCUUCCGC |
| | CACCCCCGAUAAAGCAUAACAUGGAUAUUGGAACUUGGGAUAACAAGGGU |
| | CCCGUUGCAAAAGCCCCCUCACAGGCUUUGGUUCAGAAUAUAGGUCAGCC |
| | AACCCAGGGGUCUCCUCAGCCUGUAGGUCAGCAGGCUAACAAUAGCCCAC |
| | CAGUGGCUCAGGCAUCAGUAGGGCAACAGACACAGCCAUUGCCUCCACCU |
| | CCACCACAGCCUGCCCAGCUUUCAGUCCAGCAACAGGCAGCUCAGCCAACC |
| | CGCUGGGUAGCACCUCGGAACCGUGGCAGUGGGUUCGGUCAUAAUGGGGU |
| | GGAUGGUAAUGGAGUAGGACAGUCUCAGGCUGGUUCUGGAUCUACUCCUU |
| | CAGAACCCCACCCAGUGUUGGAGAAGCUUCGGUCCAUUAAUAACUAUAAC |
| | CCCAAAGAUUUUGACUGGAAUCUGAAACAUGGCCGGGUUUUCAUCAUUAA |
| | GAGCUACUCUGAGGACGAUAUUCACCGUUCCAUUAAGUAUAAUAUUUGGU |
| | GCAGCACAGAGCAUGGUAACAAGAGACUGGAUGCUGCUUAUCGUUCCAUG |
| | AACGGGAAAGGCCCCGUUUACUUACUUUUCAGUGUCAACGGCAGUGGACA |
| | CUUCUGUGGCGUGGCAGAAAUGAAAUCUGCUGUGGACUACAACACAUGUG |
| | CAGGUGUGUGGUCCCAGGACAAAUGGAAGGGUCGUUUUGAUGUCAGGUGG |
| | AUUUUUGUGAAGGACGUUCCCAAUAGCCAACUGCGACACAUUCGCCUAGA |
| | GAACAACGAGAAUAAACCAGUGACCAACUCUAGGGACACUCAGGAAGUGC |
| | CUCUGGAAAAGGCUAAGCAGGUGUUGAAAAUUAUAGCCAGCUACAAGCAC |
| | ACCACUUCCAUUUUUGAUGACUUCUCACACUAUGAGAAACGCCAAGAGGA |
| | AGAAGAAAGUGUUAAAAAGGAACGUCAAGGUCGUGGGAAAUAAAAGGCA |
| | GUUCUACACAGACUGCAGCAACGGUUGCAUCUGCAUAUCCUAAGAGGAAA |
| | AAAUGACCUUCAAGAGAAUUAGGACUUUUUUCUUAAUUUCACUGACUUCA |
| | GAGACGAUUGCAGACUUGCAGUUUAAGUAUUGGAAUUUCACAAAAGACAU |
| | AGGACUUAACUGGAAAAUGAAAAAAAAAAGAAAAAGAAAAAACUAAACA |
| | AAAAAUCCCUCUAGGUAGUUUAGGUGAAAAAUGUCCCUUUUAUUUUGGCU |
| | UUGGUUGUGAUUUCAGAGCAUAAUGCUAUGUUUUUUUGUCUUUUUACUA |
| | UGUUUUUCGGAUUUUUAAGUCCGUAAGUGCAUACAGUUUUCUCUAAUUUU |
| | UAAACCCUUUCCUCCUCCCAUUUUGACAUUUGCACUUGGAGAACACUUGA |
| | GUUGUGAAGGUUUUGGGCAUCCACCCCAGAAAGUGGGAAUUUGAUUUUAU |
| | CCUUCCGAACUGGAAGAACAUUUUUAUGAAGAAUUUUUGUCUAGGAGAAU |
| | AUAACAGUGUUACCCAAGGUUGUGUCUUUAAGGGUGGUUCAUUUUCUCUG |
| | ACCUUUUGUUACUCAAAGUAAAGUACUAGGAGUCCUAAGAAAUGUUCUGU |
| | UCUUGUACAUUAUACUGAUUAAGUCAGGAUUAAUUUGAUUUCAAAGCUGA |
| | GAACAGUGGUAAAAACUCGUUUACAGAAAUGCAUUUUGGAAGAGAAAAA |
| | UACUGUAAAACGUGUCGUGAAUGUUUCUUCAGUUUCUUGUUCAGCCAAUG |
| | AGGAAAGGGCAUUGCCUUUCUUUUUACCAUUAAUCACUUCUCAAUAAACG |
| | UGAGAUCCUGUUGAGCAUCA (SEQ ID NO: 318) |
| SLC15A3 | AGAGUUUCUCAAGAAGCCGAGAGAGGGACAGAGGCUGAGCCUGGGGGGAG |
| NM_ | GGCUGCAGGGGACUGAGAGGGGAGUGUAGGCAGUGGGGGUGAGGGGGAC |
| 016582 | AGGGAGGAGGUGGAGAAGACGGCAAGGGGCAGCAGGGUGACUGGGGCAGA |
| 51296 | GGGAGCCCCCACCCCACGCCCAGGGCAGGCCAGGAGUCAUAAGGAGAGGG |
| | GACAACAGGAAGGGGCGACAAACGGGGAGGGGAAAGGGAGGCGGAGGGGC |
| | AGCGAAGUGAAGCAGGGAGGGUCGCAGUGUGAACAAGAGUGCGAGGAAGG |
| | GUCAAGGUGGAGGGAGGAGAGGGGAGAGUUAGGGAGAAACAGCUGCAGG |
| | AGAGGCGCGGGGAGGAAGCCUUAGGGGUGGGGGAAGGGAGAGGGGAGUU |
| | GAGAAUUAGGGAGGAGGUGGUAGAGUCCGGGUAGUGAGCGGAGGGACAG |
| | GAAGGGUAGGGCAAGAAAGGGAGAGGGGACAGGAGGGAAGGGUGGGCCA |
| | AAGCGGUGAGAAAGGAGGGCCAGCCAGUUGGGUGGGGGAGAGGGCCGAGG |
| | CCCGGGGGCAGGAGUGCAGGGCUCUGAGGCGGGGAGAGGAGAGGAGAGAA |
| | GAGCCGCGGGGGGCCCAGCCCGGAGCCAGGAUGCCCGCGCCGCGCGCCCGG |
| | GAGCAGCCCCGCGUGCCCGGGGAGCGCCAGCCGCUGCUGCCUCGCGGUGCG |
| | CGGGGCCCUCGACGGUGGCGGCGGGCGGCGGGCGCGGCCGUGCUGCUGGU |
| | GGAGAUGCUGGAGCGCGCCGCCUUCUUCGGCGUCACCGCCAACCUCGUGC |
| | UGUACCUCAACAGCACCAACUUCAACUGGACCGGCGAGCAGGCGACGCGC |
| | GCCGCGCUGGUAUUCCUGGGCGCCUCCUACCUGCUGGCGCCCGUGGGCGGC |
| | UGGCUGGCCGACGUGUACCUGGGCCGCUACCGCGCGGUCGCGCUCAGCCU |
| | GCUGCUCUACCUGGCCGCCUCGGGCCUGCUGCCCGCCACCGCCUUCCCCGA |
| | CGGCCGCAGCUCCUUCUGCGGAGAGAUGCCCGCGUCGCCGCUGGGACCUGC |
| | CUGCCCCUCGGCCGGCUGCCCGCGCUCCUCGCCCAGCCCCUACUGCGCGCC |
| | CGUCCUCUACGCGGGCCUGCUGCUACUCGGCCUGGCCGCCAGCUCCGUCCG |
| | GAGCAACCUCACCUCCUUCGGUGCCGACCAGGUGAUGGAUCUCGGCCGCG |
| | ACGCCACCCGCCGCUUCUUCAACUGGUUUUACUGGAGCAUCAACCUGGGU |
| | GCUGUGCUGUCGCUGCUGGUGGUGGCGUUUAUUCAGCAGAACAUCAGCUU |
| | CCUGCUGGGCUACAGCAUCCCUGUGGGCUGUGUGGGCCUGGCAUUUUUCA |
| | UCUUCCUCUUUGCCACCCCCGUCUUCAUCACCAAGCCCCCGAUGGGCAGCC |
| | AAGUGUCCUCUAUGCUUAAGCUCGCUCUCCAAAACUGCUGCCCCCAGCUG |
| | UGGCAACGACACUCGGCCAGAGACCGUCAAUGUGCCCGCGUGCUGGCCGA |
| | CGAGAGGUCUCCCCAGCCAGGGGCUUCCCCGCAAGAGGACAUCGCCAACU |
| | UCCAGGUGCUGGUGAAGAUCUUGCCCGUCAUGGUGACCCUGGUGCCCUAC |
| | UGGAUGGUCUACUUCCAGAUGCAGUCCACCUAUGUCCUGCAGGGUCUUCA |
| | CCUCCACAUCCCAAACAUUUUCCCAGCCAACCCGGCCAACAUCUCUGUGGC |
| | CCUGAGAGCCCAGGGCAGCAGCUACACGAUCCCGGAAGCCUGGCUCCUCCU |
| | GGCCAAUGUUGUGGUGGUGCUGAUUCUGGUCCCUCUGAAGGACCGCUUGA |
| | UCGACCCUUUACUGCUGCGGUGCAAGCUGCUUCCCUCUGCUCUGCAGAAG |
| | AUGGCGCUGGGGAUGUUCUUUGGUUUUACCUCCGUCAUUGUGGCAGGAGU |
| | CCUGGAGAUGGAGCGCUUACACUACAUCCACCACAACGAGACCGUGUCCC |
| | AGCAGAUUGGGGAGGUCCUGUACAACGCGGCACCACUGUCCAUCUGGUGG |
| | CAGAUCCCUCAGUACCUGCUCAUUGGGAUCAGUGAGAUCUUUGCCAGCAU |
| | CCCAGGCCUGGAGUUUGCCUACUCAGAGGCCCCGCGCUCCAUGCAGGGCGC |
| | CAUCAUGGGCAUCUUCUUCUGCCUGUCGGGGGUGGGCUCACUGUUGGGCU |
| | CCAGCCUAGUGGCACUGCUGUCCUUGCCCGGGGGCUGGCUGCACUGCCCCA |
| | AGGACUUUGGGAACAUCAACAAUUGCCGGAUGGACCUCUACUUCUUCCUG |
| | CUGGCUGGCAUUCAGGCCGUCACGGCUCUCCUAUUUGUCUGGAUCGCUGG |
| | ACGCUAUGAGAGGGCGUCCCAGGGCCCAGCCUCCCACAGCCGUUUCAGCA |
| | GGGACAGGGGCUGAACAGGCCCUAUUCCAGCCCCCUUGCUUCACUCUACC |
| | GGACAGACGGCAGCAGUCCCAGCUCUGGUUUCCUUCUCGGUUUAUUCUGU |
| | UAGAAUGAAAUGGUUCCCAUAAAUAAGGGGCAUGAGCCCUUCCUCACGA |
| | (SEQ ID NO: 319) |
| GOLM1 | GGAGACGCGGCGGCGCUGGACGCGGAGGCGCUGGGCGCACGGCGCGGAGC |
| NM_ | CGGCCGGAGCUCGAGGCCGGCGGCGGCGGGAGAGCGACCCGGGCGGCCUC |
| 016548 | GUAGCGGGGCCCCGGAUCCCCGAGUGGCGGCCGGAGCCUCGAAAAGAGAU |
| 511280 | UCUCAGCGCUGAUUUUGAGAUGAUGGGCUUGGGAAACGGGCGUCGCAGCA |
| | UGAAGUCGCCGCCCCUCGUGCUGGCCGCCCUGGUGGCCUGCAUCAUCGUCU |
| | UGGGCUUCAACUACUGGAUUGCGAGCUCCCGGAGCGUGGACCUCCAGACA |
| | CGGAUCAUGGAGCUGGAAGGCAGGGUCCGCAGGGCGGCUGCAGAGAGAGG |
| | CGCCGUGGAGCUGAAGAAGAACGAGUUCCAGGGAGAGCUGGAGAAGCAGC |
| | GGGAGCAGCUUGACAAAAUCCAGUCCAGCCACAACUUCCAGCUGGAGAGC |
| | GUCAACAAGCUGUACCAGGACGAAAAGGCGGUUUUGGUGAAUAACAUCAC |
| | CACAGGUGAGAGGCUCAUCCGAGUGCUGCAAGACCAGUUAAAGACCCUGC |
| | AGAGGAAUUACGGCAGGCUGCAGCAGGAUGUCCUCCAGUUUCAGAAGAAC |
| | CAGACCAACCUGGAGAGGAAGUUCUCCUACGACCUGAGCCAGUGCAUCAA |
| | UCAGAUGAAGGAGGUGAAGGAACAGUGUGAGGAGCGAAUAGAAGAGGUC |
| | ACCAAAAAGGGGAAUGAAGCUGUAGCUUCCAGAGACCUGAGUGAAAACAA |
| | CGACCAGAGACAGCAGCUCCAAGCCCUCAGUGAGCCUCAGCCCAGGCUGCA |
| | GGCAGCAGGCCUGCCACACACAGAGGUGCCACAAGGGAAGGGAAACGUGC |
| | UUGGUAACAGCAAGUCCCAGACACCAGCCCCCAGUUCCGAAGUGGUUUUG |
| | GAUUCAAAGAGACAAGUUGAGAAAGAGGAAACCAAUGAGAUCCAGGUGGU |
| | GAAUGAGGAGCCUCAGAGGGACAGGCUGCCGCAGGAGCCAGGCCGGGAGC |
| | AGGUGGUGGAAGACAGACCUGUAGGUGGAAGAGGCUUCGGGGGAGCCGGA |
| | GAACUGGGCCAGACCCCACAGGUGCAGGCUGCCCUGUCAGUGAGCCAGGA |
| | AAAUCCAGAGAUGGAGGGCCCUGAGCGAGACCAGCUUGUCAUCCCCGACG |
| | GACAGGAGGAGGAGCAGGAAGCUGCCGGGGAAGGGAGAAACCAGCAGAAA |
| | CUGAGAGGAGAAGAUGACUACAACAUGGAUGAAAAUGAAGCAGAAUCUGA |
| | GACAGACAAGCAAGCAGCCCUGGCAGGGAAUGACAGAAACAUAGAUGUUU |
| | UUAAUGUUGAAGAUCAGAAAAGAGACACCAUAAAUUUACUUGAUCAGCGU |
| | GAAAAGCGGAAUCAUACACUCUGAAUUGAACUGGAAUCACAUAUUUCACA |
| | ACAGGGCCGAAGAGAUGACUAUAAAAUGUUCAUGAGGGACUGAAUACUGA |
| | AAACUGUGAAAUGUACUAAAUAAAAUGUACAUCUGAAGAUGAUUAUUGU |
| | GAAAUUUUAGUAUGCACUUUGUGUAGGAAAAAAUGGAAUGGUCUUUUAA |
| | ACAGCUUUUGGGGGGUACUUUGGAAGUGUCUAAUAAGGUGUCACAAUUUU |
| | UGGUAGUAGGUAUUUCGUGAGAAGUUCAACACCAAAACUGGAACAUAGUU |
| | CUCCUUCAAGUGUUGGCGACAGCGGGGCUUCCUGAUUCUGGAAUAUAACU |
| | UUGUGUAAAUUAACAGCCACCUAUAGAAGAGUCCAUCUGCUGUGAAGGAG |
| | AGACAGAGAACUCUGGGUUCCGUCGUCCUGUCCACGUGCUGUACCAAGUG |
| | CUGGUGCCAGCCUGUUACCUGUUCUCACUGAAAAGUCUGGCUAAUGCUCU |
| | UGUGUAGUCACUUCUGAUUCUGACAAUCAAUCAAUCAAUGGCCUAGAGCA |
| | CUGACUGUUAACACAAACGUCACUAGCAAAGUAGCAACAGCUUUAAGUCU |
| | AAAUACAAAGCUGUUCUGUGUGAGAAUUUUUUAAAAGGCUACUUGUAUA |
| | AUAACCCUUGUCAUUUUUAAUGUACAAAACGCUAUUAAGUGGCUUAGAAU |
| | UUGAACAUUUGUGGUCUUUAUUUACUUUGCUUCGUGUGUGGGCAAAGCAA |
| | CAUCUUCCCUAAAUAUAUAUUACCAAGAAAAGCAAGAAGCAGAUUAGGUU |
| | UUUGACAAAACAAACAGGCCAAAAGGGGGCUGACCUGGAGCAGAGCAUGG |
| | UGAGAGGCAAGGCAUGAGAGGGCAAGUUUGUUGUGGACAGAUCUGUGCCU |
| | ACUUUAUUACUGGAGUAAAAGAAAACAAAGUUCAUUGAUGUCGAAGGAU |
| | AUAUACAGUGUUAGAAAUUAGGACUGUUUAGAAAAACAGGAAUACAAUG |
| | GUUGUUUUUAUCAUAGUGUACACAUUUAGCUUGUGGUAAAUGACUCACAA |
| | AACUGAUUUUAAAAUCAAGUUAAUGUGAAUUUUGAAAAUUACUACUUAA |
| | UCCUAAUUCACAAUAACAAUGGCAUUAAGGUUUGACUUGAGUUGGUUCUU |
| | AGUAUUAUUUAUGGUAAAUAGGCUCUUACCACUUGCAAAUAACUGGCCAC |
| | AUCAUUAAUGACUGACUUCCCAGUAAGGCUCUCUAAGGGGUAAGUAGGAG |
| | GAUCCACAGGAUUUGAGAUGCUAAGGCCCCAGAGAUCGUUUGAUCCAACC |
| | CUCUUAUUUUCAGAGGGGAAAAUGGGGCCUAGAAGUUACAGAGCAUCUAG |
| | CUGGUGCGCUGGCACCCCUGGCCUCACACAGACUCCCGAGUAGCUGGGAC |
| | UACAGGCACACAGUCACUGAAGCAGGCCCUGUUUGCAAUUCACGUUGCCA |
| | CCUCCAACUUAAACAUUCUUCAUAUGUGAUGUCCUUAGUCACUAAGGUUA |
| | AACUUUCCCACCCAGAAAAGGCAACUUAGAUAAAAUCUUAGAGUACUUUC |
| | AUACUCUUCUAAGUCCUCUUCCAGCCUCACUUUGAGUCCUCCUUGGGGUU |
| | GAUAGGAAUUUUCUCUUGCUUUCUCAAUAAAGUCUCUAUUCAUCUCAUGU |
| | UUAAUUUGUACGCAUAGAAUUGCUGAGAAAUAAAAUGUUCUGUUCAACUU |
| | A (SEQ ID NO: 320) |
| ITGAD | ACUUCCCCUCAACGCGCUGCUCAGGGAUGACCUUCGGCACUGUGCUUCUU |
| NM_ | CUGAGUGUCCUGGCUUCUUAUCAUGGAUUCAACCUGGAUGUGGAGGAGCC |
| 001318185 | UACGAUCUUCCAGGAGGAUGCAGGCGGCUUUGGGCAGAGCGUGGUGCAGU |
| 3681 | UCGGUGGAUCUCGACUCGUGGUGGGAGCACCCCUGGAGGUGGUGGCGGCC |
| | AACCAGACGGGACGGCUGUAUGACUGCGCAGCUGCCACCGGCAUGUGCCA |
| | GCCCAUCCCGCUGCACAUCCGCCCUGAGGCCGUGAACAUGUCCUUGGGCCU |
| | GACCCUGGCAGCCUCCACCAACGGCUCCCGGCUCCUGGCCUGUGGCCCGAC |
| | CCUGCACAGAGUCUGUGGGGAGAACUCAUACUCAAAGGGUUCCUGCCUCC |
| | UGCUGGGCUCGCGCUGGGAGAUCAUCCAGACAGUCCCCGACGCCACGCCA |
| | GAGUGUCCACAUCAAGAGAUGGACAUCGUCUUCCUGAUUGACGGCUCUGG |
| | AAGCAUUGACCAAAAUGACUUUAACCAGAUGAAGGGCUUUGUCCAAGCUG |
| | UCAUGGGCCAGUUUGAGGGCACUGACACCCUGUUUGCACUGAUGCAGUAC |
| | UCAAACCUCCUGAAGAUCCACUUCACCUUCACCCAAUUCCGGACCAGCCCG |
| | AGCCAGCAGAGCCUGGUGGAUCCCAUCGUCCAACUGAAAGGCCUGACGUU |
| | CACGGCCACGGGCAUCCUGACAGUGGUGACACAGCUAUUUCAUCAUAAGA |
| | AUGGGGCCCGAAAAAGUGCCAAGAAGAUCCUCAUUGUCAUCACAGAUGGG |
| | CAGAAGUACAAAGACCCCCUGGAAUACAGUGAUGUCAUCCCCCAGGCAGA |
| | GAAGGCUGGCAUCAUCCGCUACGCUAUCGGGGUGGGACACGCUUUCCAGG |
| | GACCCACUGCCAGGCAGGAGCUGAAUACCAUCAGCUCAGCGCCUCCGCAG |
| | GACCACGUGUUCAAGGUGGACAACUUUGCAGCCCUUGGCAGCAUCCAGAA |
| | GCAGCUGCAGGAGAAGAUCUAUGCAGUUGAGGGAACCCAGUCCAGGGCAA |
| | GCAGCUCCUUCCAGCACGAGAUGUCCCAAGAAGGCUUCAGCACAGCCCUC |
| | ACAAUGGAUGGCCUCUUCCUGGGGGCUGUGGGGAGCUUUAGCUGGUCUGG |
| | AGGUGCCUUCCUGUAUCCCCCAAAUAUGAGCCCCACCUUCAUCAACAUGU |
| | CUCAGGAGAAUGUGGACAUGAGGGACUCUUACCUGGGUUACUCCACCGAG |
| | CUAGCCCUGUGGAAGGGGGUACAGAACCUGGUCCUGGGGGCCCCCCGCUA |
| | CCAGCAUACCGGGAAGGCUGUCAUCUUCACCCAGGUGUCCAGGCAAUGGA |
| | GGAAGAAGGCCGAAGUCACAGGGACGCAGAUCGGCUCCUACUUCGGGGCC |
| | UCCCUCUGCUCUGUGGAUGUGGACAGCGAUGGCAGCACCGACCUGAUCCU |
| | CAUUGGGGCCCCCCAUUACUAUGAGCAGACCCGAGGGGGCCAGGUGUCCG |
| | UGUGUCCCUUGCCUAGGGGGCAGAGGGUGCAGUGGCAGUGUGACGCUGUU |
| | CUCCGUGGUGAGCAGGGCCACCCCUGGGGCCGCUUUGGGGCAGCCCUGAC |
| | AGUGUUGGGGGAUGUGAAUGAGGACAAGCUGAUAGACGUGGCCAUUGGG |
| | GCCCCGGGAGAGCAGGAGAACCGGGGUGCUGUCUACCUGUUUCACGGAGC |
| | CUCAGAAUCCGGCAUCAGCCCCUCCCACAGCCAGCGGAUUGCCAGCUCCCA |
| | GCUCUCCCCCAGGCUGCAGUAUUUUGGGCAGGCGCUGAGUGGGGGUCAGG |
| | ACCUCACCCAGGAUGGACUGAUGGACCUGGCCGUGGGGGCCCGGGGCCAG |
| | GUGCUCCUGCUCAGGAGUCUGCCGGUGCUGAAAGUGGGGGUGGCCAUGAG |
| | AUUCAGCCCUGUGGAGGUGGCCAAGGCUGUGUACCGGUGCUGGGAAGAGA |
| | AGCCCAGUGCCCUGGAAGCUGGGGACGCCACCGUCUGUCUCACCAUCCAG |
| | AAAAGCUCACUGGACCAGCUAGGUGACAUCCAAAGCUCUGUCAGGUUUGA |
| | UCUGGCACUGGACCCAGGUCGUCUGACUUCUCGUGCCAUUUUCAAUGAAA |
| | CCAAGAACCCCACUUUGACUCGAAGAAAAACCCUGGGACUGGGGAUUCAC |
| | UGUGAAACCCUGAAGCUGCUUUUGCCAGAUUGUGUGGAGGAUGUGGUGAG |
| | CCCCAUCAUUCUGCACCUCAACUUCUCACUGGUGAGAGAGCCCAUCCCCUC |
| | CCCCCAGAACCUGCGUCCUGUGCUGGCCGUGGGCUCACAAGACCUCUUCAC |
| | UGCUUCUCUCCCCUUCGAGAAGAACUGUGGGCAAGAUGGCCUCUGUGAAG |
| | GGGACCUGGGUGUCACCCUCAGCUUCUCAGGCCUGCAGACCCUGACCGUG |
| | GGGAGCUCCCUGGAGCUCAACGUGAUUGUGACUGUGUGGAACGCAGGUGA |
| | GGAUUCCUACGGAACCGUGGUCAGCCUCUACUAUCCAGCAGGGCUGUCGC |
| | ACCGACGGGUGUCAGGAGCCCAGAAGCAGCCCCAUCAGAGUGCCCUGCGC |
| | CUGGCAUGUGAGACAGUGCCCACUGAGGAUGAGGGCCUAAGAAGCAGCCG |
| | CUGCAGUGUCAACCACCCCAUCUUCCAUGAGGGCUCUAACGGCACCUUCA |
| | UAGUCACAUUCGAUGUCUCCUACAAGGCCACCCUGGGAGACAGGAUGCUU |
| | AUGAGGGCCAGUGCAAGCAGUGAGAACAAUAAGGCUUCAAGCAGCAAGGC |
| | CACCUUCCAGCUGGAGCUCCCGGUGAAGUAUGCAGUCUACACCAUGAUCA |
| | GCAGGCAGGAAGAAUCCACCAAGUACUUCAACUUUGCAACCUCCGAUGAG |
| | AAGAAAAUGAAAGAGGCUGAGCAUCGAUACCGUGUGAAUAACCUCAGCCA |
| | GCGAGAUCUGGCCAUCAGCAUUAACUUCUGGGUUCCUGUCCUGCUGAACG |
| | GGGUGGCUGUGUGGGAUGUGGUCAUGGAGGCCCCAUCUCAGAGUCUCCCC |
| | UGUGUUUCAGAGAGAAAACCUCCCCAGCAUUCUGACUUCCUGACCCAGAU |
| | UUCAAGAAGUCCCAUGCUGGACUGCUCCAUUGCUGACUGCCUGCAGUUCC |
| | GCUGUGACGUCCCCUCCUUCAGCGUCCAGGAGGAGCUGGAUUUCACCCUG |
| | AAGGGCAAUCUCAGUUUCGGCUGGGUCCGCGAGACAUUGCAGAAGAAGGU |
| | GUUGGUCGUGAGUGUGGCUGAAAUUACGUUCGACACAUCCGUGUACUCCC |
| | AGCUUCCAGGACAGGAGGCAUUUAUGAGAGCUCAGAUGGAGAUGGUGCUA |
| | GAAGAAGACGAGGUCUACAAUGCCAUUCCCAUCAUCAUGGGCAGCUCUGU |
| | GGGGGCUCUGCUACUGCUGGCGCUCAUCACAGCCACACUGUACAAGCUUG |
| | GCUUCUUCAAACGCCACUACAAGGAAAUGCUGGAGGACAAGCCUGAAGAC |
| | ACUGCCACAUUCAGUGGGGACGAUUUCAGCUGUGUGGCCCCAAAUGUGCC |
| | UUUGUCCUAAUAAUCCACUUUCCUGUUUAUCUCUACCACUGUGGGCUGGA |
| | CUUGCUUGCAACCAUAAAUCAACUUACAUGGAAACAACUUCUGCAUAGAU |
| | CUGCACUGGCCUAAGCAACCUACCAGGUGCUAAGCACCUUCUCGGAGAGA |
| | UAGAGAUUGUAAUGUUUUUACAUAUCUGUCCAUCUUUUUCAGCAAUGACC |
| | CACUUUUUACAGAAGCAGGCAUGGUGCCAGCAUAAAUUUUCAUAUGCUUA |
| | AGAAUUGUCACAUGAAAUGAGGAUGUUUAUAGCACACUUUCCUUGCGUGG |
| | AAGAGCUAUAACCCAGGGACCUGAGUGCCUCUCUGGGAAUAGUCGGGGGA |
| | ACCUAUUUGUGGGCAUUGAAAAAGUUUUUUCACUUUC (SEQ ID NO: 321) |
| TNFRSF1B | AGUCGAGGGCUAGCGAGCGCAGCGGAGCCUGGAGAGAAGGCGCUGGGCUG |
| NM_ | CGAGGGCGCGAGGGCGCGAGGGCAGGGGGCAACCGGACCCCGCCCGCACCC |
| 001066 | AUGGCGCCCGUCGCCGUCUGGGCCGCGCUGGCCGUCGGACUGGAGCUCUG |
| 7133 | GGCUGCGGCGCACGCCUUGCCCGCCCAGGUGGCAUUUACACCCUACGCCCC |
| | GGAGCCCGGGAGCACAUGCCGGCUCAGAGAAUACUAUGACCAGACAGCUC |
| | AGAUGUGCUGCAGCAAAUGCUCGCCGGGCCAACAUGCAAAAGUCUUCUGU |
| | ACCAAGACCUCGGACACCGUGUGUGACUCCUGUGAGGACAGCACAUACAC |
| | CCAGCUCUGGAACUGGGUUCCCGAGUGCUUGAGCUGUGGCUCCCGCUGUA |
| | GCUCUGACCAGGUGGAAACUCAAGCCUGCACUCGGGAACAGAACCGCAUC |
| | UGCACCUGCAGGCCCGGCUGGUACUGCGCGCUGAGCAAGCAGGAGGGGUG |
| | CCGGCUGUGCGCGCCGCUGCGCAAGUGCCGCCCGGGCUUCGGCGUGGCCAG |
| | ACCAGGAACUGAAACAUCAGACGUGGUGUGCAAGCCCUGUGCCCCGGGGA |
| | CGUUCUCCAACACGACUUCAUCCACGGAUAUUUGCAGGCCCCACCAGAUC |
| | UGUAACGUGGUGGCCAUCCCUGGGAAUGCAAGCAUGGAUGCAGUCUGCAC |
| | GUCCACGUCCCCCACCCGGAGUAUGGCCCCAGGGGCAGUACACUUACCCCA |
| | GCCAGUGUCCACACGAUCCCAACACACGCAGCCAACUCCAGAACCCAGCAC |
| | UGCUCCAAGCACCUCCUUCCUGCUCCCAAUGGGCCCCAGCCCCCCAGCUGA |
| | AGGGAGCACUGGCGACUUCGCUCUUCCAGUUGGACUGAUUGUGGGUGUGA |
| | CAGCCUUGGGUCUACUAAUAAUAGGAGUGGUGAACUGUGUCAUCAUGACC |
| | CAGGUGAAAAAGAAGCCCUUGUGCCUGCAGAGAGAAGCCAAGGUGCCUCA |
| | CUUGCCUGCCGAUAAGGCCCGGGGUACACAGGGCCCCGAGCAGCAGCACC |
| | UGCUGAUCACAGCGCCGAGCUCCAGCAGCAGCUCCCUGGAGAGCUCGGCC |
| | AGUGCGUUGGACAGAAGGGCGCCCACUCGGAACCAGCCACAGGCACCAGG |
| | CGUGGAGGCCAGUGGGGCCGGGGAGGCCCGGGCCAGCACCGGGAGCUCAG |
| | AUUCUUCCCCUGGUGGCCAUGGGACCCAGGUCAAUGUCACCUGCAUCGUG |
| | AACGUCUGUAGCAGCUCUGACCACAGCUCACAGUGCUCCUCCCAAGCCAGC |
| | UCCACAAUGGGAGACACAGAUUCCAGCCCCUCGGAGUCCCCGAAGGACGA |
| | GCAGGUCCCCUUCUCCAAGGAGGAAUGUGCCUUUCGGUCACAGCUGGAGA |
| | CGCCAGAGACCCUGCUGGGGAGCACCGAAGAGAAGCCCCUGCCCCUUGGA |
| | GUGCCUGAUGCUGGGAUGAAGCCCAGUUAACCAGGCCGGUGUGGGCUGUG |
| | UCGUAGCCAAGGUGGGCUGAGCCCUGGCAGGAUGACCCUGCGAAGGGGCC |
| | CUGGUCCUUCCAGGCCCCCACCACUAGGACUCUGAGGCUCUUUCUGGGCCA |
| | AGUUCCUCUAGUGCCCUCCACAGCCGCAGCCUCCCUCUGACCUGCAGGCCA |
| | AGAGCAGAGGCAGCGAGUUGUGGAAAGCCUCUGCUGCCAUGGCGUGUCCC |
| | UCUCGGAAGGCUGGCUGGGCAUGGACGUUCGGGGCAUGCUGGGGCAAGUC |
| | CCUGACUCUCUGUGACCUGCCCCGCCCAGCUGCACCUGCCAGCCUGGCUUC |
| | UGGAGCCCUUGGGUUUUUUGUUUGUUUGUUUGUUUGUUUGUUUGUUUCU |
| | CCCCCUGGGCUCUGCCCCAGCUCUGGCUUCCAGAAAACCCCAGCAUCCUUU |
| | UCUGCAGAGGGGCUUUCUGGAGAGGAGGGAUGCUGCCUGAGUCACCCAUG |
| | AAGACAGGACAGUGCUUCAGCCUGAGGCUGAGACUGCGGGAUGGUCCUGG |
| | GGCUCUGUGCAGGGAGGAGGUGGCAGCCCUGUAGGGAACGGGGUCCUUCA |
| | AGUUAGCUCAGGAGGCUUGGAAAGCAUCACCUCAGGCCAGGUGCAGUGGC |
| | UCACGCCUAUGAUCCCAGCACUUUGGGAGGCUGAGGCGGGUGGAUCACCU |
| | GAGGUUAGGAGUUCGAGACCAGCCUGGCCAACAUGGUAAAACCCCAUCUC |
| | UACUAAAAAUACAGAAAUUAGCCGGGCGUGGUGGCGGGCACCUAUAGUCC |
| | CAGCUACUCAGAAGCCUGAGGCUGGGAAAUCGUUUGAACCCGGGAAGCGG |
| | AGGUUGCAGGGAGCCGAGAUCACGCCACUGCACUCCAGCCUGGGCGACAG |
| | AGCGAGAGUCUGUCUCAAAAGAAAAAAAAAAGCACCGCCUCCAAAUGCCA |
| | ACUUGUCCUUUUGUACCAUGGUGUGAAAGUCAGAUGCCCAGAGGGCCCAG |
| | GCAGGCCACCAUAUUCAGUGCUGUGGCCUGGGCAAGAUAACGCACUUCUA |
| | ACUAGAAAUCUGCCAAUUUUUUAAAAAAGUAAGUACCACUCAGGCCAACA |
| | AGCCAACGACAAAGCCAAACUCUGCCAGCCACAUCCAACCCCCCACCUGCC |
| | AUUUGCACCCUCCGCCUUCACUCCGGUGUGCCUGCAGCCCCGCGCCUCCUU |
| | CCUUGCUGUCCUAGGCCACACCAUCUCCUUUCAGGGAAUUUCAGGAACUA |
| | GAGAUGACUGAGUCCUCGUAGCCAUCUCUCUACUCCUACCUCAGCCUAGA |
| | CCCUCCUCCUCCCCCAGAGGGGUGGGUUCCUCUUCCCCACUCCCCACCUUC |
| | AAUUCCUGGGCCCCAAACGGGCUGCCCUGCCACUUUGGUACAUGGCCAGU |
| | GUGAUCCCAAGUGCCAGUCUUGUGUCUGCGUCUGUGUUGCGUGUCGUGGG |
| | UGUGUGUAGCCAAGGUCGGUAAGUUGAAUGGCCUGCCUUGAAGCCACUGA |
| | AGCUGGGAUUCCUCCCCAUUAGAGUCAGCCUUCCCCCUCCCAGGGCCAGGG |
| | CCCUGCAGAGGGGAAACCAGUGUAGCCUUGCCCGGAUUCUGGGAGGAAGC |
| | AGGUUGAGGGGCUCCUGGAAAGGCUCAGUCUCAGGAGCAUGGGGAUAAAG |
| | GAGAAGGCAUGAAAUUGUCUAGCAGAGCAGGGGCAGGGUGAUAAAUUGU |
| | UGAUAAAUUCCACUGGACUUGAGCUUGGCAGCUGAACUAUUGGAGGGUGG |
| | GAGAGCCCAGCCAUUACCAUGGAGACAAGAAGGGUUUUCCACCCUGGAAU |
| | CAAGAUGUCAGACUGGCUGGCUGCAGUGACGUGCACCUGUACUCAGGAGG |
| | CUGAGGGGAGGAUCACUGGAGCCCAGGAGUUUGAGGCUGCAGCGAGCUAU |
| | GAUCGCGCCACUACACUCCAGCCUGAGCAACAGAGUGAGACCCUGUCUCU |
| | UAAAGAAAAAAAAAGUCAGACUGCUGGGACUGGCCAGGUUUCUGCCCACA |
| | UUGGACCCACAUGAGGACAUGAUGGAGCGCACCUGCCCCCUGGUGGACAG |
| | UCCUGGGAGAACCUCAGGCUUCCUUGGCAUCACAGGGCAGAGCCGGGAAG |
| | CGAUGAAUUUGGAGACUCUGUGGGGCCUUGGUUCCCUUGUGUGUGUGUGU |
| | UGAUCCCAAGACAAUGAAAGUUUGCACUGUAUGCUGGACGGCAUUCCUGC |
| | UUAUCAAUAAACCUGUUUGUUUUA (SEQ ID NO: 322) |
| ATG7 | AGAGAGAGCUGUGGUUGCCGGAAGUUGAGCGGCGGCAAGAAAUAAUGGCG |
| NM_ | GCAGCUACGGGGGAUCCUGGACUCUCUAAACUGCAGUUUGCCCCUUUUAG |
| 001136031 | UAGUGCCUUGGAUGUUGGGUUUUGGCAUGAGUUGACCCAGAAGAAGCUGA |
| 10533 | ACGAGUAUCGGCUGGAUGAAGCUCCCAAGGACAUUAAGGGUUAUUACUAC |
| | AAUGGUGACUCUGCUGGGCUGCCAGCUCGCUUAACAUUGGAGUUCAGUGC |
| | UUUUGACAUGAGUGCUCCCACCCCAGCCCGUUGCUGCCCAGCUAUUGGAA |
| | CACUGUAUAACACCAACACACUCGAGUCUUUCAAGACUGCAGAUAAGAAG |
| | CUCCUUUUGGAACAAGCAGCAAAUGAGAUAUGGGAAUCCAUAAAAUCAGG |
| | CACUGCUCUUGAAAACCCUGUACUCCUCAACAAGUUCCUCCUCUUGACAU |
| | UUGCAGAUCUAAAGAAGUACCACUUCUACUAUUGGUUUUGCUAUCCUGCC |
| | CUCUGUCUUCCAGAGAGUUUACCUCUCAUUCAGGGGCCAGUGGGUUUGGA |
| | UCAAAGGUUUUCACUAAAACAGAUUGAAGCACUAGAGUGUGCAUAUGAUA |
| | AUCUUUGUCAAACAGAAGGAGUCACAGCUCUUCCUUACUUCUUAAUCAAG |
| | UAUGAUGAGAACAUGGUGCUGGUUUCCUUGCUUAAACACUACAGUGAUUU |
| | CUUCCAAGGUCAAAGGACGAAGAUAACAAUUGGUGUAUAUGAUCCCUGUA |
| | ACUUAGCCCAGUACCCUGGAUGGCCUUUGAGGAAUUUUUUGGUCCUAGCA |
| | GCCCACAGAUGGAGUAGCAGUUUCCAGUCUGUUGAAGUUGUUUGCUUCCG |
| | UGACCGUACCAUGCAGGGGGCGAGAGACGUUGCCCACAGCAUCAUCUUCG |
| | AAGUGAAGCUUCCAGAAAUGGCAUUUAGCCCAGAUUGUCCUAAAGCAGUU |
| | GGAUGGGAAAAGAACCAGAAAGGAGGCAUGGGACCAAGGAUGGUGAACCU |
| | CAGUGAAUGUAUGGACCCUAAAAGGUUAGCUGAGUCAUCAGUGGAUCUAA |
| | AUCUCAAACUGAUGUGUUGGAGAUUGGUUCCUACUUUAGACUUGGACAAG |
| | GUUGUGUCUGUCAAAUGUCUGCUGCUUGGAGCCGGCACCUUGGGUUGCAA |
| | UGUAGCUAGGACGUUGAUGGGUUGGGGCGUGAGACACAUCACAUUUGUGG |
| | ACAAUGCCAAGAUCUCCUACUCCAAUCCUGUGAGGCAGCCUCUCUAUGAG |
| | UUUGAAGAUUGCCUAGGGGGUGGUAAGCCCAAGGCUCUGGCAGCAGCGGA |
| | CCGGCUCCAGAAAAUAUUCCCCGGUGUGAAUGCCAGAGGAUUCAACAUGA |
| | GCAUACCUAUGCCUGGGCAUCCAGUGAACUUCUCCAGUGUCACUCUGGAG |
| | CAAGCCCGCAGAGAUGUGGAGCAACUGGAGCAGCUCAUCGAAAGCCAUGA |
| | UGUCGUCUUCCUAUUGAUGGACACCAGGGAGAGCCGGUGGCUUCCUGCCG |
| | UCAUUGCUGCAAGCAAGAGAAAGCUGGUCAUCAAUGCUGCUUUGGGAUUU |
| | GACACAUUUGUUGUCAUGAGACAUGGUCUGAAGAAACCAAAGCAGCAAGG |
| | AGCUGGGGACUUGUGUCCAAACCACCCUGUGGCAUCUGCUGACCUCCUGG |
| | GCUCAUCGCUUUUUGCCAACAUCCCUGGUUACAAGCUUGGCUGCUACUUC |
| | UGCAAUGAUGUGGUGGCCCCAGGAGAUUCAACCAGAGACCGGACCUUGGA |
| | CCAGCAGUGCACUGUGAGUCGUCCAGGACUGGCCGUGAUUGCAGGAGCCC |
| | UGGCCGUGGAAUUGAUGGUAUCUGUUUUGCAGCAUCCAGAAGGGGGCUAU |
| | GCCAUUGCCAGCAGCAGUGACGAUCGGAUGAAUGAGCCUCCAACCUCUCU |
| | UGGGCUUGUGCCUCACCAGGUUCUUGAUCAAUAUGAACGAGAAGGAUUUA |
| | ACUUCCUAGCCAAGGUGUUUAAUUCUUCACAUUCCUUCUUAGAAGACUUG |
| | ACUGGUCUUACAUUGCUGCAUCAAGAAACCCAAGCUGCUGAGAUCUGGGA |
| | CAUGAGCGAUGAUGAGACCAUCUGAGAUGGCCCCGCUGUGGGGCUGACUU |
| | CUCCCCGGCCGCCUGCUGAGGAGCUCUCCAUCGCCAGAGCAGGACUGCUGA |
| | CCCCAGGCCUGGUGAUUCUGGGCCCCUCCUCCAUACCCCGAGGUCUGGGAU |
| | UCCCCCCUCUGCUGCCCAGGAGUGGCCAGUGUUCGGCGUUGCUCGGGAUU |
| | CAAGAUACCACCAGUUCAGAGCUAAAUAAUAACCUUGGCCUUGGCCUUGC |
| | UAUUGACCUGGGACUUGGUCCUCCAUGCAGUUUUUAUUUCUUGUCACAGU |
| | GACUGAUAGCCAUCCCCCAGGAUCCUUUCCCCUUGGCCCUGAGGGGGUGA |
| | CCCAACACAGACCAAAUGGGGAAAUGAGCAACCAGCUCCUGCCCAGAGCC |
| | ACUGCGGGAGGUGGCACCCUCAUCCCCGGAAUGUGCUGCCCACCGCACCGC |
| | AGGCUCCUCCUGUGGGGGCCCUGGGCAUGGGUGAGGGUGGGACCCCGUGA |
| | GCGCACUGCACCCUGGCCCUGGUGGAGCGGGAGGAGGAGGAGAGCCGAGC |
| | UGGGUACGAGACUAAAGGGCCCACAUGACCCAGUGACGCCAGAUUUCCAC |
| | CAAGGACUGAGUGAGCUGCUCAGACAUGGCUUUCUGCCUCCCAGCCUGUC |
| | CUCCACUGUGGGCAUAGCAUCUGUGCCUGCCUGCCUGCUUGAGGGAGAGG |
| | AGUUUCUGCUGCUGCCUUGAGCUGGGGGGAAGAGCCCAGGGGCAGAUCCU |
| | GGCAGCUGCCUGGAUGGGGCUCCUCCCUGCCCUUAUGAGCAGGCCAGGCC |
| | CAGAAAGGCCGAGCCUGGGCUGCCUUCCUGCCCCAGCCGAGGGAGGGGUC |
| | AGACGGCUCUACCAUGGGUAACUCAGGCAAGAGCUGGUUUUCCUCUUUAU |
| | UCUGGGUGUGUGCAGCUGUGAGGCCCCAACCCAGGAGAGGCCAUGGCCUA |
| | GGUACCUGUGACCACCCUGCCCCCGUGUAGAGGGCAUCGUCUUUCCUGCU |
| | AUUUUAUUCUUUCAGCUUUUGUCUUAGGCCCAGAAUCAAAGUGAAAAUUG |
| | AGUCGAGCUGACCCUUACAACAGUAGGAUUUAGUAGGGUAGAUUUCAAAU |
| | GAGGCUUCGCUUCUCCCAAAGUAGCCAGUCCAAGUUCCAGUGGCUGUCGU |
| | UCAGCUCAUGGGAGCUUCAUGGGGACACAGCCGGCACAGGUGCAGGGCCC |
| | GAGUCCGCCCACCCAGCCUGGCGCUGAAACUGCACACGUACACUAUGUGG |
| | UUUAAGAGCACUUUAUUAUUGUUCUUAAGGCUACUUUUAAGUACAAAAAA |
| | AGAUGGCCUGCCAAACCUUUUUUUUUCUUCUUCCAGGAAAAACAGGCCAC |
| | AGAGAAUGGUAUAUUACAGAUUUACACACAUGAAGAGAAGGUCAGAGCGC |
| | ACUGCAGGCAGCGCGGCUCUGGGAAGAACUUCACGGAGCCCCUUCUUAGA |
| | GCAGGGAGGGGGCUUUCUCAGUGAAAUGUUUGGUUUUCUGCUGCCUCCUC |
| | UGCCCCAGGCCCCCCUCCAGGGUACUGCCUAUCCCAGAUAGGUCAGUGCAC |
| | CAGGGACCCGGCCGCCAGCACCGCCGACCCCUCCCAGAGUGACGCCCUUGU |
| | UCACUGACAAAGAGACCUGUCCCAGGAGUGUCCUCCACCGAGCCGGUCAG |
| | CUGUGGGUGGUUUUCCUGUUACGACGCUCAGUAGCCUGUAGCAAUAACAA |
| | ACUCGUGGCUAUGAAUGCAGAUGCAGUGUUCUCAUAGAAUAACUGUUCCU |
| | GCACUUUUACAGACAAAUCUACGACAAAAAAAAAGAUCAACUUUUUUUUU |
| | CCGAACAACAAAAAAAAUGAAUGAUUACAAUAGGAAAGGGAAAAAUUAA |
| | AUAGCUACAUAUCAUUAACAAAUUAAUGUUCUUCAAAAAAUACCUACAAA |
| | UUUCUCUGUACAUUCUUUACGCACAGCGUAACGAUGGUCUCAAAAUCACC |
| | CAUAUAGAAAAGUGUUCUCAACGAUUUUUCCUACAGAAAAUAUAGGGGCC |
| | UGAAUGCCAAAGCUUGGAAGCCCAGUACAGUGGGAGUGAAAUGUGUGCGG |
| | GGCAAGGAGAAGGGCUUUUCUUUCCUCCACUUUUCAAAGGCCUGCAGCCA |
| | CUCUGUGACUACAAGAGCCAGUCCUCCGACCUUUUCACCCAGUGCCAAUU |
| | UCCAAAAUUCAACAGCUAAAAACUGUAAAACCGGGGGUCAUACGGUGUGC |
| | AGAGUCCACAAAGCCUUGCAGGUGAGGUGACCACGCCCACGUCACCUGGU |
| | CAGGUGCCAUCGUCGUGAGCCUCUGGUGGGCCAGGUGGGACACAGCACAC |
| | CCCAGGGGGAGGGGAUAGAAACGCUCAUUGACCAAAAAGGAGCAGCUGUG |
| | ACCUCCACAGCUGUGUCUGUCAUGCUUGCUUCAUCUAAUUUCUAGUUAGU |
| | AGCUAUUAAUAUAGCAAAUAAUAAAUGCAGUAAUAACAGUAUAAAGUCA |
| | GAGGAAUGUAUACUGCCUUGGCCCCAGCGUACGAGGAAGCGUAUAAAACA |
| | CCAUAUCACAGAUUGUCUGUCAGUAAUCUGCUGUUCAGCCAAGAGAGUUC |
| | AAAGGGAGCAGUUUCUGCAUGUAGGGAAGUUGGAAGACACAAACCCCACC |
| | UCCCCUGGGAGCUUGUAACAAAGCAGACAGGGAUGCAAAAAUAAAUGAUG |
| | UCAGCCUGCAGCCAAACUCCAGCAUCCCACACCGCAGCUGACCCACUGCUC |
| | AUCGCGAGGGCCUGCCAGGAGCUGGCCUCCCGCACUACUUGUGAGUAAAG |
| | UGAAUAUCAAAUACCAAUCUUAGAGUACAACUGUACCAGCAGUAAGUAUA |
| | UCUAGGACUGUAACUGACAAAAAUAAACUAAUUCUGAAAAGAA (SEQ ID |
| | NO: 323) |
| MafB | ACAGCUGCACCGCCGAGCUGCGAGCGGCUGCGAGCGAGAGAGCGUAAGAG |
| NM_005461 | CAAGAGAGCUAGAGAGCGAGCAACGGGCACUCGCCCCACGCCUCCCCUCAG |
| 9935 | CCCCACCGCGCGCUCCGCUUGCCUCUCCACCCCGCCCGACUCUACCCGGCC |
| | CGGUCCCUGCGCGGGCACAGCCCAGAGCUCUGGGGCGGUGCAGGCAGCCU |
| | CGGGACUCUCCGGCGCGCCGCCGCGUCCCCAGACAAAGGCUUGGCCGGCGG |
| | CCCCGGCCCGCUGCGCCCUCGCUCCCCGCCUCCCCAGCUCUUCUCCGCUCU |
| | UCCCCCCCGCGCUUGGCUCGGCGCGCUCCGGCCGGCCGCAAAGUUUCCCGG |
| | GCGGCAGCGGCGGCUGCGCCUCGCUUCAGCGAUGGCCGCGGAGCUGAGCA |
| | UGGGGCCAGAGCUGCCCACCAGCCCGCUGGCCAUGGAGUAUGUCAACGAC |
| | UUCGACCUGCUCAAGUUCGACGUGAAGAAGGAGCCACUGGGGCGCGCGGA |
| | GCGUCCGGGCAGGCCCUGCACACGCCUGCAGCCAGCCGGCUCGGUGUCCUC |
| | CACACCGCUCAGCACUCCGUGUAGCUCCGUGCCCUCGUCGCCCAGCUUCAG |
| | CCCGACCGAACAGAAGACACACCUCGAGGAUCUGUACUGGAUGGCGAGCA |
| | ACUACCAGCAGAUGAACCCCGAGGCGCUCAACCUGACGCCCGAGGACGCG |
| | GUGGAAGCGCUCAUCGGCUCGCACCCAGUGCCACAGCCGCUGCAAAGCUU |
| | CGACAGCUUUCGCGGCGCUCACCACCACCACCAUCACCACCACCCUCACCC |
| | GCACCACGCGUACCCGGGCGCCGGCGUGGCCCACGACGAGCUGGGCCCGCA |
| | CGCUCACCCGCACCAUCACCAUCAUCACCAAGCGUCGCCGCCGCCGUCCAG |
| | CGCCGCUAGCCCGGCGCAACAGCUGCCCACUAGCCACCCCGGGCCCGGGCC |
| | GCACGCGACGGCCUCGGCGACGGCGGCGGGCGGCAACGGCAGCGUGGAGG |
| | ACCGCUUCUCCGACGACCAGCUCGUGUCCAUGUCCGUGCGCGAGCUGAACC |
| | GCCACCUGCGGGGCUUCACCAAGGACGAGGUGAUCCGCCUGAAGCAGAAG |
| | CGGCGGACCCUGAAGAACCGGGGCUACGCCCAGUCUUGCAGGUAUAAACG |
| | CGUCCAGCAGAAGCACCACCUGGAGAAUGAGAAGACGCAGCUCAUUCAGC |
| | AGGUGGAGCAGCUUAAGCAGGAGGUGUCCCGGCUGGCCCGCGAGAGAGAC |
| | GCCUACAAGGUCAAGUGCGAGAAACUCGCCAACUCCGGCUUCAGGGAGGC |
| | GGGCUCCACCAGCGACAGCCCCUCCUCUCCCGAGUUCUUUCUGUGAGUCGU |
| | GGCCGGUCCUGGCCCCCGCCCUUGCCCCGGCCCGGACUCCCUGUCCCACGU |
| | CCCUAGUCCCAGACUACCCCGGACCCUGUCCCUGCCGCGGCCCCAGCCUUG |
| | ACCUGUUUGACUUGAGCGAGAGGGAGGAAGGGCGCGCGGGCCGCGGGCGA |
| | CGGGCGGGUGCGCGGGCGGGCAGGGGACCUUGGCUAAGGCGAGAGUAGCG |
| | CACGCCAGCGCCGCCUCCUAGACUCGAGCAGAGCCGGAGAGAGAGACGAG |
| | AGGGUGGGAGGUCCCGGAGUAACUUCUCUCCAGGCUGAAGGGCGGCGAGG |
| | CAUAGUCCCGAGAAGUCACCAAGGCCAUCUGGAGACUCCUGGCUUUCUGA |
| | ACUUUGCGCGUUAAGCCGGGACAGCUGCUUUGCUGCCCGGAGAGUAGUCC |
| | GCGCCAGGAAGAGAGCAACGAGGAAAGGAGAGGGACUCUGGCGUCCCGGC |
| | AGGCGAGAGGCGAGGCUGAGCGAAAGAAGGAAGGACAGACGGACCUGUCU |
| | GUCAGAGUUCGGAGAACACUGGCUCUCAGCCCUGAGACACAGGCCUCAGU |
| | UAGGACGCUCGGCGCCCAAAUCUCAUCAGUUUUAUUGCCUGCUCGAUUAU |
| | AUAGAAAAAUACAAAAAAUCUGCAUUAAAAAUAUUAAUCCUGCAUGCUGG |
| | ACAUGUAUGGUAAUAAUUUCUAUUUUGUACCAUUUUCUUGUUUAACUUUA |
| | GCAUGUUGUUGAUCAUGGAUCAUACUCCCCUUGUUUCUUUGGGUGAGAAG |
| | GGAUCGCAGUUUGGAAACUCCGGCGGCUGCGUGCGGGGUUUCAGUCCCAG |
| | CUGUAGGCUUGUAAAUACCCGCCCCGCCAAACCGCAUAGAGAACGUGGCA |
| | GCAAGCUGAGGGUCUUUGUUUGGGUUUAUUAUUACGGUAUUUUUGUUUG |
| | UAAGUUAAAAAGAAAAAAAAAAAGAAAAAGUUCCGGGCAUUUUGCAUCA |
| | GAAAACAACUUUGUCUUGGGGCACACUUGGAAGUUGCAUGUUUUCUUUCC |
| | UUCCCUUAUCCCCAUUCGGUCCUCUUUUUCCUCUCUCGCUUUAGUUUUCA |
| | ACCUUGUUGGUGCUGAGAGAGAGAACCGAGAGGUCCCAGUACAAGGGCAG |
| | GGCAGGGCAGGGAAGCUGCCAAGCUCCGCACCCCAGAGGAGUGUUCUGGA |
| | CUACAGCCUUGUCUUAUGGUCAAAUUGAUACCCUUAAUAAGAAAGGAAAG |
| | GAAAGGAAAACAGAUCCUCCCCUCUGCUUUUUAUUGUAACCAGAAUCACC |
| | CUGAGGUCCCUUCUGAACCCUCUGGGCCUGCGCUAAUUGUAGGAGCCACA |
| | GCGCUCCUAGGGUGAGAGGCUUAGCCAUCCCUGACCCUGGCAGUGCACUG |
| | GUAAGCAGACACUGCACUGAACCAACUGCUAUGCUCAGAAUGUACCAGAA |
| | ACCCAAACAUUGGCAAGUAAUUUUGCAACUUUCAAGUGCGUUCUUUAGAC |
| | CAAUGCAUUGCGUUUCUUUCCCUGCUUUUGAGAUAGUAGGAAGAGUUCUU |
| | GGUGGUGUCCCCCCCCUUCAAUUCUUCAGUUGUAUAGUAGUUAUAGGGAA |
| | GAUAUGGGUGUUUUUCUUUAUUAUUACUUUUUUUUUUCUGCAGGUCAGU |
| | AAAAGGAUUUAAGUUGCACUGACAAAAAUACCAAAAUAAAAGUGUAUUU |
| | UUAAGUUCCCAUUUGAAAUUGCUGGCGCUGCUGGCCGGAUGCAUUUUUGA |
| | GUUUGUAUUAGUUGAUAAAUUAACAGUAAUAACAAGAUUGUAUGAACCG |
| | CAUGGUGCUUGCAGUUUUAAAUAUUGUGGAUAUUUGUCCUGCAUCAGAAA |
| | CGAGCUUUGGUUUUUACAGAUUCAACUGUGUUGAAAUCAAACCUGCCGCA |
| | ACAGAAAUUGUUUUUAUUUCAUGUAAAAUAAGGGAUCAAUUUCAAACCCU |
| | GCUUAUGAUAUGAAAAUAUUAAAACCUAGUCUAUUGUAGUUUUAUUCAG |
| | ACUGGUUUCUGUUUUUUGGUUAUUAAAAUGGUUUCCUAUUUUGCUUAUU |
| | AAAACCAUGGAAAUGGUGUUUAUUUAGAGGA (SEQ ID NO: 324) |
| MAF | CUUGCUGGAUCAGAGGCUUUAAAAUCUUUUUUCAUCUUCUAGCUGUAGCU |
| NM_ | CGGGCUGCUUGUCGGCUUGGCCUCCCCCUCCCCCCUUUGCUCUCUGCCUCG |
| 001031804 | UCUUUCCCCAGGACUUCGCUAUUUUGCUUUUUUAAAAAAAGGCAAGAAAG |
| 4094 | AACUAAACUCCCCCCUCCCUCUCCUCCAGUCGGGCUGCACCUCUGCCUUGC |
| | ACUUUGCACAGAGGUAGAGAGCGCGCGAGGGAGAGAGAGGAAAGAAAAAA |
| | AAUAAUAAAGAGAGCCAAGCAGAAGAGGAGGCGAGAAGCAUGAAGUGUU |
| | AACUCCCCCGUGCCAAGGCCCGCGCCGCCCGGACAGACGCCCGCCGCGCCU |
| | CCAGCCCCGAGCGGACGCCGCGCGCGCCCUGCCUGCAGCCCGGGCCGGCGA |
| | GGCGAGCCCUUCCUUAUGCAAAGCGCGCAGCGGAGCGGCGAGCGGGGGAC |
| | GCCGCGCACCGGGCCGGGCUCCUCCAGCUUCGCCGCCGCAGCCACCACCGC |
| | CGCCACCGCAGCUCGCGGAGGAUCUUCCCGAGCCUGAAGCCGCCGGCUCGG |
| | CGCGCAAGGAGGCGAGCGAGCAAGGAGGGGCCGGGGCGAGCGAGGGAGCA |
| | CAUUGGCGUGAGCAGGGGGGAGGGAGGGCGGGCGCGGGGGGCGCGGGCAG |
| | GGGGGGGGGUGUGUGUGUGAGCGCGCUCGGAGGUUUCGGGCCAGCCACC |
| | GCCGCGCAAGCUAGAAGCGCCCCAGCCCGGCAAGCUGGCUCACCCGCUGGC |
| | CACCCAGCACAGCCCGCUGGCCCCUCUCCUGCAGCCCAUCUGGCGGAGCGG |
| | CGGCGGCGGCGGCGGCGGCGGCAGGAGAAUGGCAUCAGAACUGGCAAUGA |
| | GCAACUCCGACCUGCCCACCAGUCCCCUGGCCAUGGAAUAUGUUAAUGAC |
| | UUCGAUCUGAUGAAGUUUGAAGUGAAAAAGGAACCGGUGGAGACCGACCG |
| | CAUCAUCAGCCAGUGCGGCCGUCUCAUCGCCGGGGGCUCGCUGUCCUCCAC |
| | CCCCAUGAGCACGCCGUGCAGCUCGGUGCCCCCUUCCCCCAGCUUCUCGGC |
| | GCCCAGCCCGGGCUCGGGCAGCGAGCAGAAGGCGCACCUGGAAGACUACU |
| | ACUGGAUGACCGGCUACCCGCAGCAGCUGAACCCCGAGGCGCUGGGCUUC |
| | AGCCCCGAGGACGCGGUCGAGGCGCUCAUCAGCAACAGCCACCAGCUCCAG |
| | GGCGGCUUCGAUGGCUACGCGCGCGGGGCGCAGCAGCUGGCCGCGGCGGC |
| | CGGGGCCGGUGCCGGCGCCUCCUUGGGCGGCAGCGGCGAGGAGAUGGGCC |
| | CCGCCGCCGCCGUGGUGUCCGCCGUGAUCGCCGCGGCCGCCGCGCAGAGCG |
| | GCGCGGGCCCGCACUACCACCACCACCACCACCACGCCGCCGGCCACCACC |
| | ACCACCCGACGGCCGGCGCGCCCGGCGCCGCGGGCAGCGCGGCCGCCUCGG |
| | CCGGUGGCGCUGGGGGCGCGGGCGGCGGUGGCCCGGCCAGCGCUGGGGGC |
| | GGCGGCGGCGGCGGCGGCGGCGGAGGCGGCGGGGGCGCGGCGGGGGCGGG |
| | GGGCGCCCUGCACCCGCACCACGCCGCCGGCGGCCUGCACUUCGACGACCG |
| | CUUCUCCGACGAGCAGCUGGUGACCAUGUCUGUGCGCGAGCUGAACCGGC |
| | AGCUGCGCGGGGUCAGCAAGGAGGAGGUGAUCCGGCUGAAGCAGAAGAGG |
| | CGGACCCUGAAAAACCGCGGCUAUGCCCAGUCCUGCCGCUUCAAGAGGGU |
| | GCAGCAGAGACACGUCCUGGAGUCGGAGAAGAACCAGCUGCUGCAGCAAG |
| | UCGACCACCUCAAGCAGGAGAUCUCCAGGCUGGUGCGCGAGAGGGACGCG |
| | UACAAGGAGAAAUACGAGAAGUUGGUGAGCAGCGGCUUCCGAGAAAACGG |
| | CUCGAGCAGCGACAACCCGUCCUCUCCCGAGUUUUUCAUGUGAGUCUGAC |
| | ACGCGAUUCCAGCUAGCCACCCUGAUAAGUGCUCCGCGGGGGUCCGGCUC |
| | GGGUGUGGGCUUGCUAGUUCUAGAGCCAUGCUCGCCACCACCUCACCACC |
| | CCCACCCCCACCGAGUUUGGCCCCCUUGGCCCCCUACACACACACAAACCC |
| | GCACGCACACACCACACACACACACACACACACACACACACCCCACACCCU |
| | GCUCGAGUUUGUGGUGGUGGUGGCUGUUUUAAACUGGGGAGGGAAUGGG |
| | UGUCUGGCUCAUGGAUUGCCAAUCUGAAAUUCUCCAUAACUUGCUAGCUU |
| | GUUUUUUUUUUUUUUUUACACCCCCCCGCCCCACCCCCGGACUUGCACAAU |
| | GUUCAAUGAUCUCAGCAGAGUUCUUCAUGUGAAACGUUGAUCACCUUUGA |
| | AGCCUGCAUCAUUCACAUAUUUUUUCUUCUUCUUCCCCUUCAGUUCAUGA |
| | ACUGGUGUUCAUUUUCUGUGUGUGUGUGUGUUUUAUUUUGUUUGGAUUU |
| | UUUUUUUUAAUUUUACUUUUAGAGCUUGCUGUGUUGCCCACCUUUUUUCC |
| | AACCUCCACCCUCACUCCUUCUCAACCCAUCUCUUCCGAGAUGAAAGAAAA |
| | AAAAAAGCAAAGUUUUUUUUUCUUCUCCUGAGUUCUUCAUGUGAGAUUGA |
| | GCUUGCAAAGGAAAAAAAAAUGUGAAAUGUUAUAGACUUGCAGCGUGCCG |
| | AGUUCCAUCGGGUUUUUUUUUUAGCAUUGUUAUGCUAAAAUAGAGAAAA |
| | AAAUCCUCAUGAACCUUCCACAAUCAAGCCUGCAUCAACCUUCUGGGUGU |
| | GACUUGUGAGUUUUGGCCUUGUGAUGCCAAAUCUGAGAGUUUAGUCUGCC |
| | AUUAAAAAAACUCAUUCUCAUCUCAUGCAUUAUUAUGCUUGCUACUUUGU |
| | CUUAGCAACAAUGAACUAUAACUGUUUCAAAGACUUUAUGGAAAAGAGAC |
| | AUUAUAUUAAUAAAAAAAAAAAGCCUGCAUGCUGGACAUGUAUGGUAUA |
| | AUUAUUUUUUCCUUUUUUUUUCCUUUUGGCUUGGAAAUGGACGUUCGAAG |
| | ACUUAUAGCAUGGCAUUCAUACUUUUGUUUUAUUGCCUCAUGACUUUUUU |
| | GAGUUUAGAACAAAACAGUGCAACCGUAGAGCCUUCUUCCCAUGAAAUUU |
| | UGCAUCUGCUCCAAAACUGCUUUGAGUUACUCAGAACUUCAACCUCCCAA |
| | UGCACUGAAGGCAUUCCUUGUCAAAGAUACCAGAAUGGGUUACACAUUUA |
| | ACCUGGCAAACAUUGAAGAACUCUUAAUGUUUUCUUUUUAAUAAGAAUGA |
| | CGCCCCACUUUGGGGACUAAAAUUGUGCUAUUGCCGAGAAGCAGUCUAAA |
| | AUUUAUUUUUUAAAAAGAGAAACUGCCCCAUUAUUUUUGGUUUGUUUUA |
| | UUUUUAUUUUAUAUUUUUUGGCUUUUGGUCAUUGUCAAAUGUGGAAUGC |
| | UCUGGGUUUCUAGUAUAUAAUUUAAUUCUAGUUUUUAUAAUCUGUUAGCC |
| | CAGUUAAAAUGUAUGCUACAGAUAAAGGAAUGUUAUAGAUAAAUUUGAA |
| | AGAGUUAGGUCUGUUUAGCUGUAGAUUUUUUAAACGAUUGAUGCACUAA |
| | AUUGUUUACUAUUGUGAUGUUAAGGGGGGUAGAGUUUGCAAGGGGACUG |
| | UUUAAAAAAAGUAGCUUAUACAGCAUGUGCUUGCAACUUAAAUAUAAGUU |
| | GGGUAUGUGUAGUCUUUGCUAUACCACUGACUGUAUUGAAAACCAAAGUA |
| | UUAAGAGGGGAAACGCCCCUGUUUAUAUCUGUAGGGGUAUUUUACAUUCA |
| | AAAAUGUAUGUUUUUUUUUCUUUUCAAAAUUAAAGUAUUUGGGACUGAA |
| | UUGCACUAAGAUAUAACCUGCAAGCAUAUAAUACAAAAAAAAAUUGCAAA |
| | ACUGUUUAGAACGCUAAUAAAAUUUAUGCAGUUAUAAAAAUGGCAUUACU |
| | GCACAGUUUUAAGAUGAUGCAGAUUUUUUUACAGUUGUAUUGUGGUGCA |
| | GAACUGGAUUUUCUGUAACUUAAAAAAAAAUCCACAGUUUUAAAGGCAAU |
| | AAUCAGUAAAUGUUAUUUUCAGGGACUGACAUCCUGUCUUUAAAAAGAAA |
| | UGAAAAGUAAAUCUUACCACAAUAAAUAUAAAAAAAUCUUGUCAGUUACU |
| | UUUCUUUUACAUAUUUUGCUGUGCAAAAUUGUUUUAUAUCUUGAGUUACU |
| | AACUAACCACGCGUGUUGUUCCUAUGUGCUUUUCUUUCAUUUUCAAUUCU |
| | GGUUAUAUCAAGAAAAGAAUAAUCUACAAUAAUAAACGGCAUUUUUUUU |
| | UGAUUCUGUACUCAGUUUCUUAGUGUACAGUUUAACUGGGCCCAACAACC |
| | UCGUUAAAAGUGUAAAAUGCAUCCUUUUCUCCAGUGGAAGGAUUCCUGGA |
| | GGAAUAGGGAGACAGUAAUUCAGGGUGAAAUUAUAGGCUGUUUUUUGAA |
| | GUGAGGAGGCUGGCCCCAUAUACUGAUUAGCAAUAUUUAAUAUAGAUGUA |
| | AAUUAUGACCUCAUUUUUUUCUCCCCAAAGUUUUCAGUUUUCAAAUGAGU |
| | UGAGCCAUAAUUGCCCUUGGUAGGAAAAACAAAACAAAACAGUGGAACUA |
| | GGCUUCCUGAGCAUGGCCCUACACUUCUGAUCAGGAGCAAAGCCAUCCAU |
| | AGACAGAGGAGCCGGACAAAUAUGGCGCAUCAGAGGUGGCUUGCGCACAU |
| | AUGCAUUGAACGGUAAAGAGAAACAGCGCUUGCCUUUUCACUAAAGUUGA |
| | CUAUUUUUCCUUCUUCUCUUACACACCGAGAUUUUCUUGUUAGCAAGGCC |
| | UGACAAGAUUUAACAUAAACAUGACAAAUCAUAGUUGUUUGUUUUGUUU |
| | UGCUUUUCUCUUUAACACUGAAGAUCAUUUGUCUUAAAUAGGAAAAAGAA |
| | AAUCCACUCCUUACUUCCAUAUUUCCAAGUACAUAUCUGGUUUAAACUAU |
| | GUUAUCAAAUCAUAUUUCACCGUGAAUAUUCAGUGGAGAACUUCUCUACC |
| | UGGAUGAGCUAGUAAUGAUUUCAGAUCAUGCUAUCCCCAGAAAUAAAAGC |
| | AAAAAAUAAUACCUGUGUGGAAUAUAGGCUGUGCUUUGAUUUACUGGUA |
| | UUUACCCCAAAAUAGGCUGUGUAUGGGGGCUGACUUAAAGAUCCCUUGGA |
| | AAGACUCAAAACUACCUUCACUAGUAGGACUCCUAAGCGCUGACCUAUUU |
| | UUAAAUGACACAAAUUCAUGAAACUAAUGUUACAAAUUCAUGCAGUUUGC |
| | ACUCUUAGUCAUCUUCCCCUAGCACACCAAUAGAAUGUUAGACAAAGCCA |
| | GCACUGUUUUGAAAAUACAGCCAAACACGAUGACUUUUGUUUUGUUUUCU |
| | GCCGUUCUUAAAAGAAAAAAAGAUAAUAUUGCAACUCUGACUGAAAGACU |
| | UAUUUUUAAGAAAACAGGUUGUGUUUGGUGCUGCUAAGUUCUGGCCAGUU |
| | UAUCAUCUGGCCUUCCUGCCUAUUUUUUACAAAACACGAAGACAGUGUGU |
| | AACCUCGACAUUUUGACCUUCCUUUAUGUGCUAGUUUAGACAGGCUCCUG |
| | AAUCCACACUUAAUUUUGCUUAACAAAAGUCUUAAUAGUAAACCUCCCCU |
| | CAUGAGCUUGAAGUCAAGUGUUCUUGACUUCAGAUAUUUCUUUCCUUUUU |
| | UUUUUUUUUUCCUCAUCACAACUAAGAGAUACACAAACUCUGAAGAAGCA |
| | GAAAUGGAGAGAAUGCUUUUAACAAAAAAGCAUCUGAUGAAAGAUUUUA |
| | GGCAAACAUUCUCAAAAUAAGAGUGAUAUUCUGGAUGUAGUUAUUGCAGU |
| | UAUCUCAUGACAAAUGAGGCCUGGAUUGGAAGGAAAAUAUAGUUGUGUA |
| | GAAUUAAGCAUUUUGAUAGGAAUCUACAAGGUAGUUGAAUAUAAUAAGC |
| | AGGUUUGGGCCCCCAAACUUUAGAAAAUCAAAUGCAAAGGUGCUGGCAAA |
| | AAUGAGGUUUGAGUGGCUGGCUGUAAGAGAAGGUUAACUCCUAGUAAAA |
| | GGCAUUUUUAGAAAUAACAAUUACUGAAAACUUUGAAGUAUAGUGGGAG |
| | UAGCAAACAAAUACAUGUUUUUUUUUUCUUACAAAGAACUCCUAAAUCCU |
| | GAGUAAGUGCCAUUCAUUACAAUAAGUCUCUAAAUUUAAAAAAAAAAAAA |
| | UCAUAUGAGGAAAUCUAGCUUUCCCCUUUACGCUGCGUUUGAUCUUUGUC |
| | UAAAUAGUGUUAAAAUUCCUUUCAUUCCAAUUACAGAACUGAGCCCACUC |
| | GCAAGUUGGAGCCAUCAGUGGGAUACGCCACAUUUUGGAAGCCCCAGCAU |
| | CGUGUACUUACCAGUGUGUUCACAAAAUGAAAUUUGUGUGAGAGCUGUAC |
| | AUUAAAAAAAAUCAUCAUUAUUAUUAUUAUUUGCAGUCAUGGAGAACCAC |
| | CUACCCCUGACUUCUGUUUAGUCUCCUUUUUAAAUAAAAAUUACUGUGUU |
| | AGAGAAGAAGGCUAUUAAAUGUAGUAGUUAACUAUGCCUCUUGUCUGGGG |
| | GUUUCAUAGAGACCGGUAGGAAAGCGCACUCCUGCUUUUCGAUUUAUGGU |
| | GUGUGCAAGUAAACAGGUGCAUUGCUUUCAACCUGCCAUACUAGUUUUAA |
| | AAAUUCACUGAAAUUACAAAGAUACAUAUAUAUGCAUAUAUAUAAUGGA |
| | AAGUUUCCCGGAAUGCAACAAUUAGCAUUUUAAAAUCAUAUAUAGGCAUG |
| | CACAUUCUAAAUAGUACUUUUUCAUGCUUCAUUGUUUCUCUGGCAGAUAA |
| | UUUUACUAAGAAGAAAAAUAGAUAUUCGACUCCCCUUCCCUAAACAAAUC |
| | CACGGGCAGAGGCUCCAGCGGAGCCGAGCCCCCUGGUUUUCUCGUAGGCC |
| | CUAGACGGUGUUGCAUUUAUCAGUGAUGUCAAACGUGCUCAUUUGUCAGA |
| | CAUAGCUGUAAAUGAAAACAAUGUGUGGCAAAAUACAAAGUUAGUUAAA |
| | UACA (SEQ ID NO: 325) |
| CD36 | CUUUCAAUUCCUCUGGCAACAAACCACACACUGGGAUCUGACACUGUAGA |
| NM_ | GUGCUUUCUCUUCUCUUUUUUUGGGGGGGGGAGGGGGUGUGGUUGCAUAU |
| 000072 | UUAAACUCUCACGCAUUUAUGUACUGAGGACUGCAGUGUAGGACUUUCCU |
| 948 | GCAGAAUACCAUUUGAUCCUAUUAAGAAUUGUCCAAAUGUUGGAGCAUUU |
| | GAUUGAAAAAUCCUUCUUAGCCAUUUUAAAGAUAGCUUUCCAAUGAUUAG |
| | ACGAAUUGAUUCUUUCUGUGACUCAUCAGUUCAUUUCCUGUAAAAUUCAU |
| | GUCUUGCUGUUGAUUUGUGAAUAAGAACCAGAGCUUGUAGAAACCACUUU |
| | AAUCAUAUCCAGGAGUUUGCAAGAAACAGGUGCUUAACACUAAUUCACCU |
| | CCUGAACAAGAAAAAUGGGCUGUGACCGGAACUGUGGGCUCAUCGCUGGG |
| | GCUGUCAUUGGUGCUGUCCUGGCUGUGUUUGGAGGUAUUCUAAUGCCAGU |
| | UGGAGACCUGCUUAUCCAGAAGACAAUUAAAAAGCAAGUUGUCCUCGAAG |
| | AAGGUACAAUUGCUUUUAAAAAUUGGGUUAAAACAGGCACAGAAGUUUAC |
| | AGACAGUUUUGGAUCUUUGAUGUGCAAAAUCCACAGGAAGUGAUGAUGAA |
| | CAGCAGCAACAUUCAAGUUAAGCAAAGAGGUCCUUAUACGUACAGAGUUC |
| | GUUUUCUAGCCAAGGAAAAUGUAACCCAGGACGCUGAGGACAACACAGUC |
| | UCUUUCCUGCAGCCCAAUGGUGCCAUCUUCGAACCUUCACUAUCAGUUGG |
| | AACAGAGGCUGACAACUUCACAGUUCUCAAUCUGGCUGUGGCAGCUGCAU |
| | CCCAUAUCUAUCAAAAUCAAUUUGUUCAAAUGAUCCUCAAUUCACUUAUU |
| | AACAAGUCAAAAUCUUCUAUGUUCCAAGUCAGAACUUUGAGAGAACUGUU |
| | AUGGGGCUAUAGGGAUCCAUUUUUGAGUUUGGUUCCGUACCCUGUUACUA |
| | CCACAGUUGGUCUGUUUUAUCCUUACAACAAUACUGCAGAUGGAGUUUAU |
| | AAAGUUUUCAAUGGAAAAGAUAACAUAAGUAAAGUUGCCAUAAUCGACAC |
| | AUAUAAAGGUAAAAGGAAUCUGUCCUAUUGGGAAAGUCACUGCGACAUGA |
| | UUAAUGGUACAGAUGCAGCCUCAUUUCCACCUUUUGUUGAGAAAAGCCAG |
| | GUAUUGCAGUUCUUUUCUUCUGAUAUUUGCAGGUCAAUCUAUGCUGUAUU |
| | UGAAUCCGACGUUAAUCUGAAAGGAAUCCCUGUGUAUAGAUUUGUUCUUC |
| | CAUCCAAGGCCUUUGCCUCUCCAGUUGAAAACCCAGACAACUAUUGUUUC |
| | UGCACAGAAAAAAUUAUCUCAAAAAAUUGUACAUCAUAUGGUGUGCUAGA |
| | CAUCAGCAAAUGCAAAGAAGGGAGACCUGUGUACAUUUCACUUCCUCAUU |
| | UUCUGUAUGCAAGUCCUGAUGUUUCAGAACCUAUUGAUGGAUUAAACCCA |
| | AAUGAAGAAGAACAUAGGACAUACUUGGAUAUUGAACCUAUAACUGGAUU |
| | CACUUUACAAUUUGCAAAACGGCUGCAGGUCAACCUAUUGGUCAAGCCAU |
| | CAGAAAAAAUUCAAGUAUUAAAGAAUCUGAAGAGGAACUAUAUUGUGCCU |
| | AUUCUUUGGCUUAAUGAGACUGGGACCAUUGGUGAUGAGAAGGCAAACAU |
| | GUUCAGAAGUCAAGUAACUGGAAAAAUAAACCUCCUUGGCCUGAUAGAAA |
| | UGAUCUUACUCAGUGUUGGUGUGGUGAUGUUUGUUGCUUUUAUGAUUUC |
| | AUAUUGUGCAUGCAGAUCGAAAACAAUAAAAUAAGUAAGUAUGUACCAAA |
| | AAAUAUUGCUUCAAUAAUAUUAGCUUAUAUAUUACUUGUUUUCACUUUAU |
| | CAAAGAGAAGUUACAUAUUAGGCCAUAUAUAUUUCUAGACAUGUCUAGCC |
| | ACUGAUCAUUUUUAAAUAUAGGUAAAUAAACCUAUAAAUAUUAUCACGCA |
| | GAUCACUAAAGUAUAUCUUUAAUUCUGGGAGAAAUGAGAUAAAAGAUGU |
| | ACUUGUGACCAUUGUAACAAUAGCACAAAUAAAGCACUUGUGCCAAAGUU |
| | GUCCAAAAAA (SEQ ID NO: 326) |
| PDL 1 | AGUUCUGCGCAGCUUCCCGAGGCUCCGCACCAGCCGCGCUUCUGUCCGCCU |
| NM_ | GCAGGGCAUUCCAGAAAGAUGAGGAUAUUUGCUGUCUUUAUAUUCAUGAC |
| 001267706 | CUACUGGCAUUUGCUGAACGCCCCAUACAACAAAAUCAACCAAAGAAUUU |
| 29126 | UGGUUGUGGAUCCAGUCACCUCUGAACAUGAACUGACAUGUCAGGCUGAG |
| | GGCUACCCCAAGGCCGAAGUCAUCUGGACAAGCAGUGACCAUCAAGUCCU |
| | GAGUGGUAAGACCACCACCACCAAUUCCAAGAGAGAGGAGAAGCUUUUCA |
| | AUGUGACCAGCACACUGAGAAUCAACACAACAACUAAUGAGAUUUUCUAC |
| | UGCACUUUUAGGAGAUUAGAUCCUGAGGAAAACCAUACAGCUGAAUUGGU |
| | CAUCCCAGAACUACCUCUGGCACAUCCUCCAAAUGAAAGGACUCACUUGG |
| | UAAUUCUGGGAGCCAUCUUAUUAUGCCUUGGUGUAGCACUGACAUUCAUC |
| | UUCCGUUUAAGAAAAGGGAGAAUGAUGGAUGUGAAAAAAUGUGGCAUCC |
| | AAGAUACAAACUCAAAGAAGCAAAGUGAUACACAUUUGGAGGAGACGUAA |
| | UCCAGCAUUGGAACUUCUGAUCUUCAAGCAGGGAUUCUCAACCUGUGGUU |
| | UAGGGGUUCAUCGGGGCUGAGCGUGACAAGAGGAAGGAAUGGGCCCGUGG |
| | GAUGCAGGCAAUGUGGGACUUAAAAGGCCCAAGCACUGAAAAUGGAACCU |
| | GGCGAAAGCAGAGGAGGAGAAUGAAGAAAGAUGGAGUCAAACAGGGAGCC |
| | UGGAGGGAGACCUUGAUACUUUCAAAUGCCUGAGGGGCUCAUCGACGCCU |
| | GUGACAGGGAGAAAGGAUACUUCUGAACAAGGAGCCUCCAAGCAAAUCAU |
| | CCAUUGCUCAUCCUAGGAAGACGGGUUGAGAAUCCCUAAUUUGAGGGUCA |
| | GUUCCUGCAGAAGUGCCCUUUGCCUCCACUCAAUGCCUCAAUUUGUUUUC |
| | UGCAUGACUGAGAGUCUCAGUGUUGGAACGGGACAGUAUUUAUGUAUGAG |
| | UUUUUCCUAUUUAUUUUGAGUCUGUGAGGUCUUCUUGUCAUGUGAGUGUG |
| | GUUGUGAAUGAUUUCUUUUGAAGAUAUAUUGUAGUAGAUGUUACAAUUU |
| | UGUCGCCAAACUAAACUUGCUGCUUAAUGAUUUGCUCACAUCUAGUAAAA |
| | CAUGGAGUAUUUGUAAGGUGCUUGGUCUCCUCUAUAACUACAAGUAUACA |
| | UUGGAAGCAUAAAGAUCAAACCGUUGGUUGCAUAGGAUGUCACCUUUAUU |
| | UAACCCAUUAAUACUCUGGUUGACCUAAUCUUAUUCUCAGACCUCAAGUG |
| | UCUGUGCAGUAUCUGUUCCAUUUAAAUAUCAGCUUUACAAUUAUGUGGUA |
| | GCCUACACACAUAAUCUCAUUUCAUCGCUGUAACCACCCUGUUGUGAUAA |
| | CCACUAUUAUUUUACCCAUCGUACAGCUGAGGAAGCAAACAGAUUAAGUA |
| | ACUUGCCCAAACCAGUAAAUAGCAGACCUCAGACUGCCACCCACUGUCCU |
| | UUUAUAAUACAAUUUACAGCUAUAUUUUACUUUAAGCAAUUCUUUUAUUC |
| | AAAAACCAUUUAUUAAGUGCCCUUGCAAUAUCAAUCGCUGUGCCAGGCAU |
| | UGAAUCUACAGAUGUGAGCAAGACAAAGUACCUGUCCUCAAGGAGCUCAU |
| | AGUAUAAUGAGGAGAUUAACAAGAAAAUGUAUUAUUACAAUUUAGUCCA |
| | GUGUCAUAGCAUAAGGAUGAUGCGAGGGGAAAACCCGAGCAGUGUUGCCA |
| | AGAGGAGGAAAUAGGCCAAUGUGGUCUGGGACGGUUGGAUAUACUUAAAC |
| | AUCUUAAUAAUCAGAGUAAUUUUCAUUUACAAAGAGAGGUCGGUACUUAA |
| | AAUAACCCUGAAAAAUAACACUGGAAUUCCUUUUCUAGCAUUAUAUUUAU |
| | UCCUGAUUUGCCUUUGCCAUAUAAUCUAAUGCUUGUUUAUAUAGUGUCUG |
| | GUAUUGUUUAACAGUUCUGUCUUUUCUAUUUAAAUGCCACUAAAUUUUAA |
| | AUUCAUACCUUUCCAUGAUUCAAAAUUCAAAAGAUCCCAUGGGAGAUGGU |
| | UGGAAAAUCUCCACUUCAUCCUCCAAGCCAUUCAAGUUUCCUUUCCAGAA |
| | GCAACUGCUACUGCCUUUCAUUCAUAUGUUCUUCUAAAGAUAGUCUACAU |
| | UUGGAAAUGUAUGUUAAAAGCACGUAUUUUUAAAAUUUUUUUCCUAAAU |
| | AGUAACACAUUGUAUGUCUGCUGUGUACUUUGCUAUUUUUAUUUAUUUUA |
| | GUGUUUCUUAUAUAGCAGAUGGAAUGAAUUUGAAGUUCCCAGGGCUGAGG |
| | AUCCAUGCCUUCUUUGUUUCUAAGUUAUCUUUCCCAUAGCUUUUCAUUAU |
| | CUUUCAUAUGAUCCAGUAUAUGUUAAAUAUGUCCUACAUAUACAUUUAGA |
| | CAACCACCAUUUGUUAAGUAUUUGCUCUAGGACAGAGUUUGGAUUUGUUU |
| | AUGUUUGCUCAAAAGGAGACCCAUGGGCUCUCCAGGGUGCACUGAGUCAA |
| | UCUAGUCCUAAAAAGCAAUCUUAUUAUUAACUCUGUAUGACAGAAUCAUG |
| | UCUGGAACUUUUGUUUUCUGCUUUCUGUCAAGUAUAAACUUCACUUUGAU |
| | GCUGUACUUGCAAAAUCACAUUUUCUUUCUGGAAAUUCCGGCAGUGUACC |
| | UUGACUGCUAGCUACCCUGUGCCAGAAAAGCCUCAUUCGUUGUGCUUGAA |
| | CCCUUGAAUGCCACCAGCUGUCAUCACUACACAGCCCUCCUAAGAGGCUUC |
| | CUGGAGGUUUCGAGAUUCAGAUGCCCUGGGAGAUCCCAGAGUUUCCUUUC |
| | CCUCUUGGCCAUAUUCUGGUGUCAAUGACAAGGAGUACCUUGGCUUUGCC |
| | ACAUGUCAAGGCUGAAGAAACAGUGUCUCCAACAGAGCUCCUUGUGUUAU |
| | CUGUUUGUACAUGUGCAUUUGUACAGUAAUUGGUGUGACAGUGUUCUUUG |
| | UGUGAAUUACAGGCAAGAAUUGUGGCUGAGCAAGGCACAUAGUCUACUCA |
| | GUCUAUUCCUAAGUCCUAACUCCUCCUUGUGGUGUUGGAUUUGUAAGGCA |
| | CUUUAUCCCUUUUGUCUCAUGUUUCAUCGUAAAUGGCAUAGGCAGAGAUG |
| | AUACCUAAUUCUGCAUUUGAUUGUCACUUUUUGUACCUGCAUUAAUUUAA |
| | UAAAAUAUUCUUAUUUAUUUUGUUACUUGGUACACCAGCAUGUCCAUUUU |
| | CUUGUUUAUUUUGUGUUUAAUAAAAUGUUCAGUUUAACAUCCCA (SEQ ID |
| | NO: 327) |
| PD1 | GCUCACCUCCGCCUGAGCAGUGGAGAAGGCGGCACUCUGGUGGGGCUGCU |
| NM_ | CCAGGCAUGCAGAUCCCACAGGCGCCCUGGCCAGUCGUCUGGGCGGUGCU |
| 005018 | ACAACUGGGCUGGCGGCCAGGAUGGUUCUUAGACUCCCCAGACAGGCCCU |
| 5133 | GGAACCCCCCCACCUUCUCCCCAGCCCUGCUCGUGGUGACCGAAGGGGACA |
| | ACGCCACCUUCACCUGCAGCUUCUCCAACACAUCGGAGAGCUUCGUGCUA |
| | AACUGGUACCGCAUGAGCCCCAGCAACCAGACGGACAAGCUGGCCGCCUU |
| | CCCCGAGGACCGCAGCCAGCCCGGCCAGGACUGCCGCUUCCGUGUCACACA |
| | ACUGCCCAACGGGCGUGACUUCCACAUGAGCGUGGUCAGGGCCCGGCGCA |
| | AUGACAGCGGCACCUACCUCUGUGGGGCCAUCUCCCUGGCCCCCAAGGCGC |
| | AGAUCAAAGAGAGCCUGCGGGCAGAGCUCAGGGUGACAGAGAGAAGGGCA |
| | GAAGUGCCCACAGCCCACCCCAGCCCCUCACCCAGGCCAGCCGGCCAGUUC |
| | CAAACCCUGGUGGUUGGUGUCGUGGGCGGCCUGCUGGGCAGCCUGGUGCU |
| | GCUAGUCUGGGUCCUGGCCGUCAUCUGCUCCCGGGCCGCACGAGGGACAA |
| | UAGGAGCCAGGCGCACCGGCCAGCCCCUGAAGGAGGACCCCUCAGCCGUGC |
| | CUGUGUUCUCUGUGGACUAUGGGGAGCUGGAUUUCCAGUGGCGAGAGAAG |
| | ACCCCGGAGCCCCCCGUGCCCUGUGUCCCUGAGCAGACGGAGUAUGCCACC |
| | AUUGUCUUUCCUAGCGGAAUGGGCACCUCAUCCCCCGCCCGCAGGGGCUC |
| | AGCUGACGGCCCUCGGAGUGCCCAGCCACUGAGGCCUGAGGAUGGACACU |
| | GCUCUUGGCCCCUCUGACCGGCUUCCUUGGCCACCAGUGUUCUGCAGACCC |
| | UCCACCAUGAGCCCGGGUCAGCGCAUUUCCUCAGGAGAAGCAGGCAGGGU |
| | GCAGGCCAUUGCAGGCCGUCCAGGGGCUGAGCUGCCUGGGGGCGACCGGG |
| | GCUCCAGCCUGCACCUGCACCAGGCACAGCCCCACCACAGGACUCAUGUCU |
| | CAAUGCCCACAGUGAGCCCAGGCAGCAGGUGUCACCGUCCCCUACAGGGA |
| | GGGCCAGAUGCAGUCACUGCUUCAGGUCCUGCCAGCACAGAGCUGCCUGC |
| | GUCCAGCUCCCUGAAUCUCUGCUGCUGCUGCUGCUGCUGCUGCUGCUGCC |
| | UGCGGCCCGGGGCUGAAGGCGCCGUGGCCCUGCCUGACGCCCCGGAGCCUC |
| | CUGCCUGAACUUGGGGGCUGGUUGGAGAUGGCCUUGGAGCAGCCAAGGUG |
| | CCCCUGGCAGUGGCAUCCCGAAACGCCCUGGACGCAGGGCCCAAGACUGG |
| | GCACAGGAGUGGGAGGUACAUGGGGCUGGGGACUCCCCAGGAGUUAUCUG |
| | CUCCCUGCAGGCCUAGAGAAGUUUCAGGGAAGGUCAGAAGAGCUCCUGGC |
| | UGUGGUGGGCAGGGCAGGAAACCCCUCCACCUUUACACAUGCCCAGGCAG |
| | CACCUCAGGCCCUUUGUGGGGCAGGGAAGCUGAGGCAGUAAGCGGGCAGG |
| | CAGAGCUGGAGGCCUUUCAGGCCCAGCCAGCACUCUGGCCUCCUGCCGCCG |
| | CAUUCCACCCCAGCCCCUCACACCACUCGGGAGAGGGACAUCCUACGGUCC |
| | CAAGGUCAGGAGGGCAGGGCUGGGGUUGACUCAGGCCCCUCCCAGCUGUG |
| | GCCACCUGGGUGUUGGGAGGGCAGAAGUGCAGGCACCUAGGGCCCCCCAU |
| | GUGCCCACCCUGGGAGCUCUCCUUGGAACCCAUUCCUGAAAUUAUUUAAA |
| | GGGGUUGGCCGGGCUCCCACCAGGGCCUGGGUGGGAAGGUACAGGCGUUC |
| | CCCCGGGGCCUAGUACCCCCGCCGUGGCCUAUCCACUCCUCACAUCCACAC |
| | ACUGCACCCCCACUCCUGGGGCAGGGCCACCAGCAUCCAGGCGGCCAGCAG |
| | GCACCUGAGUGGCUGGGACAAGGGAUCCCCCUUCCCUGUGGUUCUAUUAU |
| | AUUAUAAUUAUAAUUAAAUAUGAGAGCAUGCUAA (SEQ ID NO: 328) |
| LILRB1 | GCAUUUCUCUUCUGUGCUCACUGCCACACGCAGCUCAGCCUGGGCGGCAC |
| NM_ | AGCCAGAUGCGAGAUGCGUCUCUGCUGAUCUGAGUCUGCCUGCAGCAUGG |
| 001081637 | ACCUGGGUCUUCCCUGAAGCAUCUCCAGGGCUGGAGGGACGACUGCCAUG |
| 10859 | CACCGAGGGCUCAUCCAUCCACAGAGCAGGGCAGUGGGAGGAGACGCCAU |
| | GACCCCCAUCCUCACGGUCCUGAUCUGUCUCGGGCUGAGUCUGGGCCCCCG |
| | GACCCACGUGCAGGCAGGGCACCUCCCCAAGCCCACCCUCUGGGCUGAACC |
| | AGGCUCUGUGAUCACCCAGGGGAGUCCUGUGACCCUCAGGUGUCAGGGGG |
| | GCCAGGAGACCCAGGAGUACCGUCUAUAUAGAGAAAAGAAAACAGCACCC |
| | UGGAUUACACGGAUCCCACAGGAGCUUGUGAAGAAGGGCCAGUUCCCCAU |
| | CCCAUCCAUCACCUGGGAACACACAGGGCGGUAUCGCUGUUACUAUGGUA |
| | GCGACACUGCAGGCCGCUCAGAGAGCAGUGACCCCCUGGAGCUGGUGGUG |
| | ACAGGAGCCUACAUCAAACCCACCCUCUCAGCCCAGCCCAGCCCCGUGGUG |
| | AACUCAGGAGGGAAUGUAACCCUCCAGUGUGACUCACAGGUGGCAUUUGA |
| | UGGCUUCAUUCUGUGUAAGGAAGGAGAAGAUGAACACCCACAAUGCCUGA |
| | ACUCCCAGCCCCAUGCCCGUGGGUCGUCCCGCGCCAUCUUCUCCGUGGGCC |
| | CCGUGAGCCCGAGUCGCAGGUGGUGGUACAGGUGCUAUGCUUAUGACUCG |
| | AACUCUCCCUAUGAGUGGUCUCUACCCAGUGAUCUCCUGGAGCUCCUGGU |
| | CCUAGGUGUUUCUAAGAAGCCAUCACUCUCAGUGCAGCCAGGUCCUAUCG |
| | UGGCCCCUGAGGAGACCCUGACUCUGCAGUGUGGCUCUGAUGCUGGCUAC |
| | AACAGAUUUGUUCUGUAUAAGGACGGGGAACGUGACUUCCUUCAGCUCGC |
| | UGGCGCACAGCCCCAGGCUGGGCUCUCCCAGGCCAACUUCACCCUGGGCCC |
| | UGUGAGCCGCUCCUACGGGGGCCAGUACAGAUGCUACGGUGCACACAACC |
| | UCUCCUCCGAGUGGUCGGCCCCCAGCGACCCCCUGGACAUCCUGAUCGCAG |
| | GACAGUUCUAUGACAGAGUCUCCCUCUCGGUGCAGCCGGGCCCCACGGUG |
| | GCCUCAGGAGAGAACGUGACCCUGCUGUGUCAGUCACAGGGAUGGAUGCA |
| | AACUUUCCUUCUGACCAAGGAGGGGGCAGCUGAUGACCCAUGGCGUCUAA |
| | GAUCAACGUACCAAUCUCAAAAAUACCAGGCUGAAUUCCCCAUGGGUCCU |
| | GUGACCUCAGCCCAUGCGGGGACCUACAGGUGCUACGGCUCACAGAGCUC |
| | CAAACCCUACCUGCUGACUCACCCCAGUGACCCCCUGGAGCUCGUGGUCUC |
| | AGGACCGUCUGGGGGCCCCAGCUCCCCGACAACAGGCCCCACCUCCACAUC |
| | UGCAGGCCCUGAGGACCAGCCCCUCACCCCCACCGGGUCGGAUCCCCAGAG |
| | UGGUCUGGGAAGGCACCUGGGGGUUGUGAUCGGCAUCUUGGUGGCCGUCA |
| | UCCUACUGCUCCUCCUCCUCCUCCUCCUCUUCCUCAUCCUCCGACAUCGAC |
| | GUCAGGGCAAACACUGGACAUCGACCCAGAGAAAGGCUGAUUUCCAACAU |
| | CCUGCAGGGGCUGUGGGGCCAGAGCCCACAGACAGAGGCCUGCAGUGGAG |
| | GUCCAGCCCAGCUGCCGAUGCCCAGGAAGAAAACCUCUAUGCUGCCGUGA |
| | AGCACACACAGCCUGAGGAUGGGGUGGAGAUGGACACUCGGCAGAGCCCA |
| | CACGAUGAAGACCCCCAGGCAGUGACGUAUGCCGAGGUGAAACACUCCAG |
| | ACCUAGGAGAGAAAUGGCCUCUCCUCCUUCCCCACUGUCUGGGGAAUUCC |
| | UGGACACAAAGGACAGACAGGCGGAAGAGGACAGGCAGAUGGACACUGAG |
| | GCUGCUGCAUCUGAAGCCCCCCAGGAUGUGACCUACGCCCAGCUGCACAGC |
| | UUGACCCUCAGACGGGAGGCAACUGAGCCUCCUCCAUCCCAGGAAGGGCC |
| | CUCUCCAGCUGUGCCCAGCAUCUACGCCACUCUGGCCAUCCACUAGCCCAG |
| | GGGGGGACGCAGACCCCACACUCCAUGGAGUCUGGAAUGCAUGGGAGCUG |
| | CCCCCCCAGUGGACACCAUUGGACCCCACCCAGCCUGGAUCUACCCCAGGA |
| | GACUCUGGGAACUUUUAGGGGUCACUCAAUUCUGCAGUAUAAAUAACUAA |
| | UGUCUCUACAAUUUUGAAAUAAAGCAACAGACUUCUCAAUAAUCAAUGAA |
| | GUAGCUGAGAAAACUAAGUCAGAAAGUGCAUUAAACUGAAUCACAAUGUA |
| | AAUAUUACACAUCAAGCGAUGAAACUGGAAAACUACAAGCCACGAAUGAA |
| | UGAAUUAGGAAAGAAAAAAAGUAGGAAAUGAAUGAUCUUGGCUUUCCUA |
| | UAAGAAAUUUAGGGCAGGGCACGGUGGCUCACGCCUGUAAUUCCAGCACU |
| | UUGGGAGGCCGAGGCGGGCAGAUCACGAGUUCAGGAGAUCGAGACCAUCU |
| | UGGCCAACAUGGUGAAACCCUGUCUCUCCUAAAAAUACAAAAAUUAGCUG |
| | GAUGUGGUGGCAGUGCCUGUAAUCCCAGCUAUUUGGGAGGCUGAGGCAGG |
| | AGAAUCGCUUGAACCAGGGAGUCAGAGGUUUCAGUGAGCCAAGAUCGCAC |
| | CACUGCUCUCCAGCCUGGCGACAGAGGGAGACUCCAUCUCAAAUUAAAAA |
| | AAAAAAAAAAAAAGAAAGAAAAAGAGAAAAAAGAAAUUUAGAAGAAUAA |
| | CAAGUUAUUCCAAAUGAAGGCGUAAGAAAGGGAAUAAUAACAAUAAUAA |
| | GAGGAGUUGUUCAUGAGGAAAAACCAAAGCUUGAAAAUUCAACAAAGCCA |
| | GUGAAGCUCAUUCUUGAAAACAUGAAUCACACUCAUGAAUUCUAACUACA |
| | AUGAAAAAGAGAAAGAAAGAGCAGGCAUGCAUUUCCAUAUGGGAGUGAGC |
| | CAGCAGACAGCCCUACAGAUCGUACACACGUUUUCCAAAACUAACAAUGG |
| | AACAGGCGGCAAACCUAUGCCAAUAUACUAGAAAUUGCAGAUUAAAUAGA |
| | UGAAAUAUUCUAAACUGGAGUUUACAUAAUGAACAUAAGAGUAAUCAGA |
| | GAAUCUGACUCAUUUUAGAUGUGUGUGUGUGUGUAUAUAUAUGUGUGUG |
| | UGUGUGAAAAACAUUGACUAUAAUAAAAAUAAUCUCGAGUUCA (SEQ ID |
| | NO: 329) |
| SIGLEC7 | GCAGUUCCUGAGAGAAGAACCCUGAGGAACAGACGUUCCCUCGCGGCCCU |
| NM_ | GGCACCUCCAACCCCAGAUAUGCUGCUGCUGCUGCUGCUGCCCCUGCUCUG |
| 001277201 | GGGGAGGGAGAGGGUGGAAGGACAGAAGAGUAACCGGAAGGAUUACUCGC |
| 27036 | UGACGAUGCAGAGUUCCGUGACCGUGCAAGAGGGCAUGUGUGUCCAUGUG |
| | CGCUGCUCCUUCUCCUACCCAGUGGACAGCCAGACUGACUCUGACCCAGUU |
| | CAUGGCUACUGGUUCCGGGCAGGGAAUGAUAUAAGCUGGAAGGCUCCAGU |
| | GGCCACAAACAACCCAGCUUGGGCAGUGCAGGAGGAAACUCGGGACCGAU |
| | UCCACCUCCUUGGGGACCCACAGACCAAAAAUUGCACCCUGAGCAUCAGA |
| | GAUGCCAGAAUGAGUGAUGCGGGGAGAUACUUCUUUCGUAUGGAGAAAGG |
| | AAAUAUAAAAUGGAAUUAUAAAUAUGACCAGCUCUCUGUGAACGUGACAG |
| | GGUAACCUGACUGAGUCCUGGGCAGAUGAUAACCCCCGACACCAUGGCCU |
| | GGCUGCCCACUCCUCAGGGGAGGAAAGAGAGAUCCAGUAUGCACCCCUCA |
| | GCUUUCAUAAGGGGGAGCCUCAGGACCUAUCAGGACAAGAAGCCACCAAC |
| | AAUGAGUACUCAGAGAUCAAGAUCCCCAAGUAAGAAAAUGCAGAGGCUCG |
| | GGCUUGUUUGAGGGUUCACGACCCCUCCAGCAAAGGAGUCUGAGGCUGAU |
| | UCCAGUAGAAUUAGCAGCCCUCAAUGCUGUGCAACAAGACAUCAGAACUU |
| | AUUCCUCUUGUCUAACUGAAAAUGCAUGCCUGAUGACCAAACUCUCCCUU |
| | UCCCCAUCCAAUCGGUCCACACUCCCCGCCCUGGCCUCUGGUACCCACCAU |
| | UCUCCUCUGUACUUCUCUAAGGAUGACUACUUUAGAUUCCGAAUAUAGUG |
| | AGAUUGUAACGUG (SEQ ID NO: 330) |

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

1. A modified immune cell comprising a chimeric antigen receptor (CAR), wherein the CAR comprises:
(a) an extracellular domain;
(b) a transmembrane domain; and
(c) one or more intracellular domains;
wherein the extracellular domain is or comprises an anti-mesothelin antigen binding domain comprising an amino acid sequence that is at least 80% identical to a sequence selected from Table 3; and
wherein the modified immune cell is or comprises a macrophage, monocyte, dendritic cell, or stem cell.

2. The modified immune cell of claim 1, wherein the extracellular domain is or comprises an scFv, VHH antibody, centyrin, darpin, or nanobody.

3. The modified immune cell of claim 1 or 2, wherein the transmembrane domain is or comprises a CD8, CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain.

4. The modified immune cell of any one of claims 1-3, wherein the one or more intracellular domains comprise: a CD3ζ, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPα, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, IL1R, IL2R, IL3R, IL4R, IL5R, IL6R, IL7R, IL8R, IL9R, IL10R, IL11R, IL12R, IL13R, IL14R, IL15R, IL17R, IFNaR, IFNgR, TNFR, CSF1R, CSF2R, Dap10, CD36, Dectin-1, ICOSL, or Syk intracellular domain, a portion of any of the foregoing, or combinations thereof.

5. The modified immune cell of any one of claims 1-4, wherein the one or more intracellular domains comprise a CD3ζ intracellular domain or an FcRγ intracellular domain.

6. The modified immune cell of any one of claims 1-5, wherein the CAR further comprises an extracellular leader domain.

7. The modified immune cell of claim 6, wherein the extracellular leader domain comprises a CD8a extracellular leader domain.

8. The modified immune cell of any one of claims 1-7, wherein the CAR further comprises an extracellular hinge domain.

9. The modified immune cell of claim 8, wherein the extracellular hinge domain comprises: a CD8 extracellular hinge domain, a CD8a extracellular hinge domain, a CD28 extracellular hinge domain, a DNGR-1 extracellular hinge domain, a Dectin-1 extracellular hinge domain, or an IgG4 extracellular hinge domain.

10. The modified immune cell of claim 8 or 9, wherein the CAR comprises, from N-terminus to C-terminus:
a CD8a leader domain, an anti-mesothelin antigen binding domain, a CD8 extracellular hinge domain, a CD8 transmembrane domain, and a CD3ζ intracellular domain;
a CD8a leader domain, an anti-mesothelin antigen binding domain, a CD28 extracellular hinge domain, a CD28 transmembrane domain, and a CD3ζ intracellular domain;
a CD8a leader domain, an anti-mesothelin antigen binding domain, a CD28 extracellular hinge domain, a CD28 transmembrane domain, and an FcRγ intracellular domain;
a CD8a leader domain, an anti-mesothelin antigen binding domain, a CD8 extracellular hinge domain, a CD8 transmembrane domain, a CD3ζ intracellular domain, a P2A cleavage peptide, and a CD40 ligand (CD40L);
a CD8a leader domain, an anti-mesothelin antigen binding domain, a CD28 extracellular hinge domain, a CD28 transmembrane domain, a CD3ζ intracellular domain, a P2A cleavage peptide, and a CD40 ligand (CD40L);
a CD8a leader domain, an anti-mesothelin antigen binding domain, a CD28 extracellular hinge domain, a CD28 transmembrane domain, a MyD88 intracellular domain, and a CD3ζ intracellular domain;
a CD8a leader domain, an anti-mesothelin antigen binding domain, a CD28 extracellular hinge domain, a CD28 transmembrane domain, a MyD88 intracellular domain, a CD40 intracellular domain, and a CD3ζ intracellular domain;
a CD8a leader domain, an anti-mesothelin antigen binding domain, a CD28 extracellular hinge domain, a CD28 transmembrane domain, a truncated MyD88 intracellular domain, a CD40 intracellular domain, and a CD3ζ intracellular domain;
a CD8a leader domain, an anti-mesothelin antigen binding domain, a CD28 extracellular hinge domain, a CD28 transmembrane domain, an FcRγ intracellular domain, a P2A cleavage peptide, and a CD40 ligand (CD40L);
a CD8a leader domain, an anti-mesothelin antigen binding domain, a CD28 extracellular hinge domain, a CD28 transmembrane domain, a MyD88 intracellular domain, and an FcRγ intracellular domain;
a CD8a leader domain, an anti-mesothelin antigen binding domain, a CD28 extracellular hinge domain, a CD28 transmembrane domain, a MyD88 intracellular domain, a CD40 intracellular domain, and an FcRγ intracellular domain; or
a CD8a leader domain, an anti-mesothelin antigen binding domain, a CD28 extracellular hinge domain, a CD28 transmembrane domain, a truncated MyD88 intracellular domain, a CD40 intracellular domain, and an FcRγ intracellular domain.

11. The modified immune cell of claim 10, wherein the CAR has or comprises:
(a) an amino acid sequence selected from Table 2;
(b) an amino acid sequence that differs from a sequence selected from Table 2 by no more than five substitutions, additions, or deletions; or
(c) an amino acid sequence that is at least 80% identical to a sequence selected from Table 2.

12. A pharmaceutical composition comprising a modified immune cell of any one of the previous claims.

13. The pharmaceutical composition of claim 12, wherein the pharmaceutical composition comprises a pharmaceutically acceptable carrier.

14. A nucleic acid construct comprising one or more nucleic acid sequences encoding:
(a) an extracellular binding domain;
(b) a transmembrane domain; and
(c) one or more intracellular domains;
wherein the extracellular domain is or comprises an anti-mesothelin antigen binding domain comprising an amino acid sequence that is at least 80% identical to a sequence selected from Table 5; and
wherein the nucleic acid construct encodes a chimeric antigen receptor (CAR) comprising (a) through (c).

15. The nucleic acid construct of claim 14, further comprising one or more nucleic acid sequences encoding:
(d) one or more extracellular leader domains,
(e) one or more extracellular hinge domains,
(f) one or more cleavage peptides,
or combinations thereof.

16. The nucleic acid construct of claim 15, wherein the cleavage peptide is or comprises a P2A, F2A, E2A or T2A peptide.

17. The nucleic acid construct of claim 15 or 16, wherein the nucleic acid construct encodes, from N-terminus to C-terminus:
a CD8a leader domain, an anti-mesothelin antigen binding domain, a CD8 extracellular hinge domain, a CD8 transmembrane domain, and a CD3ζ intracellular domain;
a CD8a leader domain, an anti-mesothelin antigen binding domain, a CD28 extracellular hinge domain, a CD28 transmembrane domain, and a CD3ζ intracellular domain;
a CD8a leader domain, an anti-mesothelin antigen binding domain, a CD28 extracellular hinge domain, a CD28 transmembrane domain, and an FcRγ intracellular domain;
a CD8a leader domain, an anti-mesothelin antigen binding domain, a CD8 extracellular hinge domain, a CD8 transmembrane domain, a CD3ζ intracellular domain, a P2A cleavage peptide, and a CD40 ligand (CD40L);
a CD8a leader domain, an anti-mesothelin antigen binding domain, a CD28 extracellular hinge domain, a CD28 transmembrane domain, a CD3ζ intracellular domain, a P2A cleavage peptide, and a CD40 ligand (CD40L);
a CD8a leader domain, an anti-mesothelin antigen binding domain, a CD28 extracellular hinge domain, a CD28 transmembrane domain, a MyD88 intracellular domain, and a CD3ζ intracellular domain;
a CD8a leader domain, an anti-mesothelin antigen binding domain, a CD28 extracellular hinge domain, a CD28 transmembrane domain, a MyD88 intracellular domain, a CD40 intracellular domain, and a CD3ζ intracellular domain;
a CD8a leader domain, an anti-mesothelin antigen binding domain, a CD28 extracellular hinge domain, a CD28 transmembrane domain, a truncated MyD88 intracellular domain, a CD40 intracellular domain, and a CD3ζ intracellular domain;
a CD8a leader domain, an anti-mesothelin antigen binding domain, a CD28 extracellular hinge domain, a CD28 transmembrane domain, an FcRγ intracellular domain, a P2A cleavage peptide, and a CD40 ligand (CD40L);
a CD8a leader domain, an anti-mesothelin antigen binding domain, a CD28 extracellular hinge domain, a CD28 transmembrane domain, a MyD88 intracellular domain, and an FcRγ intracellular domain;
a CD8a leader domain, an anti-mesothelin antigen binding domain, a CD28 extracellular hinge domain, a CD28 transmembrane domain, a MyD88 intracellular domain, a CD40 intracellular domain, and an FcRγ intracellular domain; or
a CD8a leader domain, an anti-mesothelin antigen binding domain, a CD28 extracellular hinge domain, a CD28 transmembrane domain, a truncated MyD88 intracellular domain, a CD40 intracellular domain, and an FcRγ intracellular domain.

18. The nucleic acid construct of claim 17, wherein the nucleic acid construct has or comprises:
(a) a nucleotide sequence selected from Table 4;
(b) a nucleotide sequence that differs from a sequence selected from Table 4 by no more than five substitutions, additions, or deletions; or
(c) a nucleotide acid sequence that is at least 80% identical to a sequence selected from Table 4.

19. The nucleic acid construct of claim 14, further comprising one or more introns, wherein the one or more introns comprise one or more inhibitory nucleic acids, and wherein the one or more inhibitory nucleic acids encode one or more inhibitory RNAs.

20. The nucleic acid construct of claim 19, wherein the one or more inhibitory RNAs are or comprise one or more shRNA.

21. The nucleic acid construct of claim 20, wherein the one or more shRNA comprise a guide strand.

22. The nucleic acid construct of claim 21, wherein the guide strand comprises a nucleic acid sequence that is reverse complementary to a target gene transcript comprising a target nucleic acid sequence.

23. The nucleic acid construct of claim 22, wherein the target gene transcript encodes human ATG7, C/EBP-alpha, C/EBP-beta, CD32b, CD36, CLEC1A, FATS, GOLM1, HAVCR2, ITGAD, KLF4, KLF6, LILRB1, LILRB2, LILRB4, MAF, MafB, PD1, PD-LI, PIK3CG, PIK3CG, PPARα, PPARγ, PTGS2, SIGLEC10, SIRPα, SLAMF3, SLAMF4, SLC15A3, STAT3, STAT6, TNFRSF1B, TOX, TREM2, YTHDF2, or ZFP36.

24. The nucleic acid construct of claim 22 or 23, wherein the target gene transcript encodes a human anti-phagocytic receptor selected from the group consisting of: SIRPα, LILRB1, SIGLEC10, PD1, SLAMF3, SLAMF4, CLEC1A, and CD32b.

25. The nucleic acid construct of any one of claims 22-24, wherein the target gene transcript encodes human SIRPα.

26. A pharmaceutical composition comprising the nucleic acid construct of any one of claims 14-25.

27. The pharmaceutical composition of claim 26, wherein the pharmaceutical composition comprises a pharmaceutically acceptable carrier.

28. A method of treating a disease or disorder in a subject, the method comprising:
administering to the subject a therapeutically effective amount of the pharmaceutical composition of any one of claim 12, 13, 26, or 27,
wherein at least one sign or symptom of the disease or disorder is improved in the subject after administration.

29. The method of claim 28, wherein the step of administering is or comprises transarterial, subcutaneous, intravenous, intradermal, intratumoral, intranodal, intramedullary, intramuscular, or intraperitoneal delivery.

30. A method of modifying an immune cell, the method comprising:
delivering to the immune cell a nucleic acid construct of any one of claims 14-25, thereby producing a modified immune cell,
wherein the modified immune cell is or comprises a macrophage, monocyte, dendritic cell, or stem cell.

31. The method of claim 30, wherein the nucleic acid construct comprises DNA or messenger RNA (mRNA).

32. The method of claim 30 or 31, wherein the nucleic acid construct comprises a modification selected from: a modified nucleotide, an alteration to the 5′ untranslated region (UTR), an alteration to the 3′ UTR, a cap structure, a poly(A) tail, or combinations thereof.

33. The method of claim 32, wherein the cap structure comprises AGCap1, m6AGCap1, or Anti-Reverse Cap Analog (ARCA).

34. The method of claim 32 or 33, wherein the modified nucleotide comprises pseudouridine (PsU), 5-methoxyuridine (5moU), 5-methylcytidine/pseudouridine (5meC PsU), N1-methyl-pseudouridine (N1mPsU), or combinations thereof.

35. The method of any one of claims 30-34, wherein the nucleic acid construct is a purified nucleic acid construct.

36. The method of claim 35, wherein the purified nucleic acid construct is produced by a method comprising silica membrane purification, high performance liquid chromatography (HPLC), Dynabeads, LiCl precipitation, phenol-chloroform extraction, resin based purification, polyA isolation, RNeasy, or combinations thereof.

37. The method of any one of claims 30-36, wherein the nucleic acid construct is codon-optimized.

38. The method of claim 37, wherein the nucleic acid construct is codon-optimized for expression in a stem cell, monocyte, macrophage, or dendritic cell.

39. The method of any one of claims 30-38, wherein the delivering comprises electroporation or transfection with the nucleic acid construct.

40. The method of any one of claims 30-38, wherein the nucleic acid construct is encapsulated within a delivery vehicle.

41. The method of claim 40, wherein the delivery vehicle is or comprises a liposome, a lipid nanoparticle, a polymer, an adeno-associated viral (AAV) vector, an adenoviral vector, a retroviral vector or combinations thereof.

42. The method of claim 41, wherein the liposome or lipid nanoparticle comprises one or more cationic lipids, one or more non-cationic lipids, one or more cholesterol-based lipids, one or more PEG-modified lipids, or combinations thereof.

43. The method of claim 41, wherein the retroviral vector comprises a lentiviral vector or a gammaretroviral vector.

44. The method of claim 43, wherein the lentiviral vector is packaged with a Vpx protein.

45. The method of claim 41, wherein the adenoviral vector comprises an Ad2 vector or an Ad5 vector.

46. The method of claim 45, wherein the Ad5 vector comprises an Ad5f35 adenoviral vector.

47. The method of any one of claims 30-46, the method further comprising delivering to the immune cell an additional payload.

48. The method of claim 47, wherein the additional payload is or comprises a pathogen recognition receptor agonist, polyinosinic:polycytidylic acid (poly I:C), a TLR7/8 agonist, a CpG oligodeoxynucleotide, a NOD-like receptor (NLR) agonist, a RIG-I-like receptor (RLR) agonist, a C-type lectins receptor (CLR) agonist, a cytosolic DNA sensing, the cyclic GMP-AMP synthase-stimulator of interferon genes (cGAS-STING) agonist, an interferon-inducible protein 16 (IFI16) agonist, a DEAD-box helicase 41 (DDX41) agonist, an LRR binding FLII interacting protein 1 (LRRFIP1) agonist, an absent in melanoma 2 (AIM2) agonist, an aryl hydrocarbon receptor (AhR) ligand, or combinations thereof.

49. The method of claim 47 or 48, wherein the nucleic acid construct and the additional payload are encapsulated within the delivery vehicle.


SEQUENCE LISTING (331 sequences)

SEQ ID NO: 1 - protein, 448 aa, synthetic construct
MALPVTALLLPLALLLHAARPGSQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 445 aa, synthetic construct
MALPVTALLLPLALLLHAARPGSQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 375 aa, synthetic construct
MALPVTALLLPLALLLHAARPGSQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQ

SEQ ID NO: 4 - protein, 734 aa, synthetic construct
MALPVTALLLPLALLLHAARPQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGATNFSLLKQAGDVEENPGPMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 731 aa, synthetic construct
MALPVTALLLPLALLLHAARPQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGATNFSLLKQAGDVEENPGPMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 6 - protein, 615 aa, synthetic construct
MALPVTALLLPLALLLHAARPQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLIEVMYPPPYLDNEKSNGTIIHVKGKHLCPSPLFPGPSKPFWVLVVVGGVLACYSLLVTVAFIIFWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 677 aa, synthetic construct
MALPVTALLLPLALLLHAARPQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLIEVMYPPPYLDNEKSNGTIIHVKGKHLCPSPLFPGPSKPFWVLVVVGGVLACYSLLVTVAFIIFWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 680 aa, synthetic construct
MALPVTALLLPLALLLHAARPQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLTTTPAPRPPTPAPTIASQPLSLRPEACRPAAGGAVHTRGLDFACDIYIWAPLAGTCGVLLLSLVITLYCMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 450 aa, synthetic construct
MALPVTALLLPLALLLHAARPGSEVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVLTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 609 aa, synthetic construct
MALPVTALLLPLALLLHAARPGSQVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVLIEVMYPPPYLDNEKSNGTIIHVKGKHLCPSPLFPGPSKPFWVLVVVGGVLACYSLLVTVAFIIFWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIKKVAKKPTNKAPHPKQEPQEINFPDDLPGSNTAAPVQETLHGCQPVTQEDGKESRISVQERQRLKIQVRKAAITSYEKSDGVYTGLSTRNQETYETLKHEKPPQ

SEQ ID NO: 11 - protein, 445 aa, synthetic construct
MALPVTALLLPLALLLHAARPEVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVLIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 733 aa, synthetic construct
MALPVTALLLPLALLLHAARPEVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVLIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGATNFSLLKQAGDVEENPGPMIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 13 - protein, 617 aa, synthetic construct
MALPVTALLLPLALLLHAARPEVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVLIEVMYPPPYLDNEKSNGTIIHVKGKHLCPSPLFPGPSKPFWVLVVVGGVLACYSLLVTVAFIIFWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 679 aa, synthetic construct
MALPVTALLLPLALLLHAARPEVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVLIEVMYPPPYLDNEKSNGTIIHVKGKHLCPSPLFPGPSKPFWVLVVVGGVLACYSLLVTVAFIIFWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 679 aa, synthetic construct; identical to SEQ ID NO: 14

SEQ ID NO: 16 - protein, 375 aa, synthetic construct
MALPVTALLLPLALLLHAARPEVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVLIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQ

SEQ ID NO: 17 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 18 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 19 - protein, 246 aa, synthetic construct
QVQLVQSGAEVKKPGAPVKVSCKASGYTFTGYYMHWVRQAPGQGLEWMGWINPNSGGTNYAQKFQGRVTMTRDTSISTAYMELSRLRSDDTAVYYCARGEWDGSYYYDYWGQGTLVTVSSGGGGSGGGGSGGGGSGGGGSDIVLTQTPSSLSASVGDRVTITCRASQSINTYLNWYQHKPGKAPKLLIYAASSLQSGVPSRFSGSGSGTDFTLTISSLQPEDFATYYCQQSFSPLTFGGGTKLEIK

SEQ ID NO: 20 - protein, 242 aa, synthetic construct
QVQLVESGGGLVQPGGSLRLSCAASGFTFSSYWMHWVRQVPGKGLVWVSRINTDGSTTTYADSVEGRFTISRDNAKNTLYLQMNSLRDDDTAVYYCVGGHWAVWGQGTTVTVSSGGGGSGGGGSGGGGSGGGGSDIQMTQSPSTLSASVGDRVTITCRASQSISDRLAWYQQKPGKAPKLLIYKASSLESGVPSRFSGSGSGTEFTLTISSLQPDDFAVYYCQQYGHLPMYTFGQGTKVEIK

SEQ ID NO: 21 - protein, 241 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 22 - protein, 253 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARYRLIAVAGDYYYYGMDVWGQGTMVTVSSGGGGSGGGGSGGGGSGGGGSDIQMTQSPSSVSASVGDRVTITCRASQGVGRWLAWYQQKPGTAPKLLIYAASTLQSGVPSRFSGSGSGTDFTLTINNLQPEDFATYYCQQANSFPLTFGGGTRLEIK

SEQ ID NO: 23 - protein, 250 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARWKVSSSSPAFDYWGQGTLVTVSSGGGGSGGGGSGGGGSGGGGSEIVLTQSPATLSLSPGERAILSCRASQSVYTKYLGWYQQKPGQAPRLLIYDASTRATGIPDRFSGSGSGTDFTLTINRLEPEDFAVYYCQHYGGSPLITFGQGTRLEIK

SEQ ID NO: 24 - protein, 246 aa, synthetic construct
QVQLQQSGAEVKKPGASVKVSCKTSGYPFTGYSLHWVRQAPGQGLEWMGWINPNSGGTNYAQKFQGRVTMTRDTSISTAYMELSRLRSDDTAVYYCARDHYGGNSLFYWGQGTLVTVSSGGGGSGGGGSGGGGSGGGGSDIQLTQSPSSISASVGDTVSITCRASQDSGTWLAWYQQKPGKAPNLLMYDASTLEDGVPSRFSGSASGTEFTLTVNRLQPEDSATYYCQQYNSYPLTFGGGTKVDIK

SEQ ID NO: 25 - protein, 248 aa, synthetic construct
QVQLVQSGAEVKKPGASVEVSCKASGYTFTSYYMHWVRQAPGQGLEWMGIINPSGGSTGYAQKFQGRVTMTRDTSTSTVHMELSSLRSEDTAVYYCARGGYSSSSDAFDIWGQGTMVTVSSGGGGSGGGGSGGGGSGGGGSDIQMTQSPPSLSASVGDRVTITCRASQDISSALAWYQQKPGTPPKLLIYDASSLESGVPSRFSGSGSGTDFTLTISSLQPEDFATYYCQQFSSYPLTFGGGTRLEIK

SEQ ID NO: 26 - protein, 255 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVAGGIYYYYGMDVWGQGTTITVSSGGGGSGGGGSGGGGSGGGGSDIVMTQTPDSLAVSLGERATISCKSSHSVLYNRNNKNYLAWYQQKPGQPPKLLFYWASTRKSGVPDRFSGSGSGTDFTLTISSLQPEDFATYFCQQTQTFPLTFGQGTRLEIN

SEQ ID NO: 27 - protein, 241 aa, synthetic construct
QVQLQQSGAEVKKPGASVKVSCKASGYTFTGYYMHWVRQAPGQGLEWMGWINPNSGGTNYAQNFQGRVTMTRDTSISTAYMELRRLRSDDTAVYYCASGWDFDYWGQGTLVTVSSGGGGSGGGGSGGGGSGGGGSDIRMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 28 - protein, 246 aa, synthetic construct
QVQLVQSGAEVKKPGASVKVSCKASGYTFTGYYMHWVRQAPGQGLEWMGRINPNSGGTNYAQKFQGRVTMTTDTSTSTAYMELRSLRSDDTAVYYCARTTTSYAFDIWGQGTMVTVSSGGGGSGGGGSGGGGSGGGGSDIQLTQSPSTLSASVGDRVTITCRASQSISTWLAWYQQKPGKAPNLLIYKASTLESGVPSRFSGSGSGTEFTLTISSLQPDDFATYYCQQYNTYSPYTFGQGTKLEIK

SEQ ID NO: 29 - protein, 249 aa, synthetic construct
QVQLVQSGGGLVKPGGSLRLSCEASGFIFSDYYMGWIRQAPGKGLEWVSYIGRSGSSMYYADSVKGRFTFSRDNAKNSLYLQMNSLRAEDTAVYYCAASPVVAATEDFQHWGQGTLVTVSSGGGGSGGGGSGGGGSGGGGSDIVMTQTPATLSLSPGERATLSCRASQSVTSNYLAWYQQKPGQAPRLLLFGASTRATGIPDRFSGSGSGTDFTLTINRLEPEDFAMYYCQQYGSAPVTFGQGTKLEIK

SEQ ID NO: 30 - protein, 249 aa, synthetic construct
QVQLVQSGAEVRAPGASVKISCKASGFTFRGYYIHWVRQAPGQGLEWMGIINPSGGSRAYAQKFQGRVTMTRDTSTSTVYMELSSLRSDDTAMYYCARTASCGGDCYYLDYWGQGTLVTVSSGGGGSGGGGSGGGGSGGGGSDIQMTQSPPTLSASVGDRVTITCRASENVNIWLAWYQQKPGKAPKLLIYKSSSLASGVPSRFSGSGSGAEFTLTISSLQPDDFATYYCQQYQSYPLTFGGGTKVDIK

SEQ ID NO: 31 - protein, 244 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVL

SEQ ID NO: 32 - protein, 246 aa, synthetic construct
EVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQEPAVSVALGQTVRITCQGDSLRSYYASWYQQKPGQAPVLVIFGRSRRPSGIPDRFSGSSSGNTASLIITGAQAEDEADYYCNSRDNTANHYVFGTGTKLTVL

SEQ ID NO: 33 - protein, 246 aa, synthetic construct
EVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVL

SEQ ID NO: 34 - protein, 251 aa, synthetic construct
QVQLVQSGGGLVQPGGSLRLSCAASGFTFSSYWMHWVRQAPGKGLVWVSRINSDGSSTSYADSVKGRFTISRDNAKNTLYLQMNSLRAEDTAVYYCVRTGWVGSYYYYMDVWGKGTTVTVSSGGGGSGGGGSGGGGSGGGGSEIVLTQSPGTLSLSPGERATLSCRASQSVSSNYLAWYQQKPGQPPRLLIYDVSTRATGIPARFSGGGSGTDFTLTISSLEPEDFAVYYCQQRSNWPPWTFGQGTKVEIK

SEQ ID NO: 35 - protein, 250 aa, synthetic construct
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKGYSRYYYYGMDVWGQGTTVTVSSGGGGSGGGGSGGGGSGGGGSEIVMTQSPATLSLSPGERAILSCRASQSVYTKYLGWYQQKPGQAPRLLIYDASTRATGIPDRFSGSGSGTDFTLTINRLEPEDFAVYYCQHYGGSPLITFGQGTKVDIK

SEQ ID NO: 36 - protein, 249 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKREAAAGHDWYFDLWGRGTLVTVSSGGGGSGGGGSGGGGSGGGGSDIRVTQSPSSLSASVGDRVTITCRASQSISSYLNWYQQKPGKAPKLLIYAASSLQSGVPSRFSGSGSGTDFTLTISSLQPEDFATYYCQQSYSIPLTFGQGTKVEIK

SEQ ID NO: 37 - DNA, 1347 bp, synthetic construct
  source 1..1347: mol_type = other DNA
atggccttgcctgtcaccgctctgcttctgccgctggccctcttgctgcacgccgcgcgtccagggagccaggtgcagctggtccagtccgggggtggactggtgcagcccggccgtagcctgcgcctgtcttgcgcagcgtccggcttcacattcgacgattatgccatgcactgggtccggcaggccccaggtaagggtcttgaatgggtgtctggtattagctggaactccggcagtatcggctatgccgattccgtaaagggtcggtttaccatttcccgcgacaacgccaagaacagcctctacctgcagatgaactctctgcgtgcggaagatacagcagtatactattgcgccaaagacggctcatcttcctggtcctggggctatttcgactactggggacaggggactctggtgacggtgtctagcggcggtggaggtagtggtggcggtggctctgggggtgggggctccagctctgagctgacacaggacccagctgtgtccgtggcgctcggtcagactgttcgcaccacttgtcaaggggacgcattgcggtcatactatgcctcttggtaccaacagaagccaggccaggctcccatgctcgttatctatggaaagaacaatcgtccatcaggtatccctgatcgtttctccggctctgacagtggggacaccgctagcctgaccatcaccggcgcgcaggctgaggacgaggccgactactattgtaacagtcgggactcctctggttacccagttttcggcaccggcaccaaggtgacagtgctgaccacgacccccgcccccaggccccctacacccgcacctaccatcgctagccagcctctctccctgcgcccggaggcgtgcaggcctgctgccggaggcgctgtacacacccgcggactggacttcgcctgtgatatctacatttgggctcccctggcggggacatgcggcgttttgctgttgtccctcgtaattaccctgtattgccgtgttaagttctctcggagtgctgatgcaccagcgtatcaacagggccagaatcagctgtacaacgagctgaatctcgggaggagagaagagtacgacgtactggacaagcgtcgggggagggatcccgagatgggcggtaagccccgcagaaagaatcctcaagaaggcctgtataacgagttgcagaaggacaaaatggcagaggcatatagtgaaatcgggatgaagggtgagaggcgccgtggcaaaggccacgatggactctaccagggcctgtctactgctacaaaggacacgtacgacgctcttcatatgcaagccctccctccccgctaa

SEQ ID NO: 38 - DNA, 1338 bp, synthetic construct
  source 1..1338: mol_type = other DNA
atggcgcttccggtgaccgcgctcctgcttcctctggcgctgttgctgcatgccgctagaccgggctcccaagtgcagctcgtccaaagtggtggcgggctcgttcaacccggccgctccctgcggctgtcttgtgcggcatctggtttcaccttcgatgactatgcgatgcactgggtcagacaggctccgggaaaggggctcgaatgggtgtctggcatctcctggaactccggcagcatcggctacgctgattctgtcaagggcagattcaccatcagccgcgacaacgctaagaactccctgtatctccagatgaacagcctgagggcggaagacacggccgtgtattactgcgccaaggacggcagctcctcatggagctggggatattttgactactggggtcaaggcaccctggtgaccgtaagcagtggtggcgggggatccggcggtgggggctccggcggagggggctccagcagtgagctgactcaagatcctgctgtgagcgtggccttgggtcagacagttcgcaccacatgccaaggcgacgcccttcgctcttattacgcttcctggtaccagcaaaaacctgggcaggcacctatgctcgtcatctatgggaagaataaccgtccatctggcatccctgaccgcttctctggctccgatagtggcgacaccgcttctctgaccatcaccggggctcaggctgaggacgaggcggactattactgtaacagtcgcgactccagcggctaccctgtcttcggtactggcacaaaagtgaccgtcctgattgaagtgatgtaccctccgccttacctggacaacgagaagagcaacggcacgattatccatgtgaaaggcaaacacctctgcccttccccgctgttcccgggccccagtaagcccttctgggtcctcgtggtcgtaggcggtgtactggcttgttatagcctgctcgtcaccgtcgccttcattatcttctgggtccgcgtaaagttcagccgcagcgccgatgcccctgcctaccaacagggccaaaatcagctgtataacgagctcaacctgggcagaagggaggaatacgacgtgctggacaaaaggcgtggacgcgatcctgagatgggcggaaaacccaggcgtaagaacccccaggagggactgtataacgagctgcagaaggataaaatggccgaggcttattctgagatcgggatgaagggcgaaagacggcgtggcaagggccatgacggtctctaccaaggtctgtctaccgccaccaaggatacctatgacgccctccatatgcaggccttgcctccacgctaa

SEQ ID NO: 39 - DNA, 1128 bp, synthetic construct
  source 1..1128: mol_type = other DNA
atggctctgccagtcaccgcactcctgttgcccctcgccctgctcctgcacgcagcccgcccaggtagtcaagtccagctggtgcaaagcggcgggggactggtgcaacctgggagaagtttgcgccttagttgtgcggcttctggattcacattcgacgattatgccatgcattgggttcgccaggctccaggtaagggcttggaatgggtttctggtatctcttggaactcagggtctatcgggtacgccgacagtgttaagggccggttcaccatcagccgggataacgcaaaaaactctctgtatctccagatgaacagtctgagggcagaggatactgccgtttattactgtgctaaggatggctccagctcatggtcctgggggtatttcgactactggggccagggcacactggtgaccgtcagctctggaggcggtggctccgggggtggcggatcaggcggaggcggttccagcagtgagctgacccaagacccagccgtttctgtggccttggggcagacagttcggactacctgccagggcgacgctctccgcagctattacgcttcatggtaccaacagaaacccgggcaggcccccatgctggtcatttacgggaagaacaatagacccagcggcattccagatcgcttttctggcagcgactccggcgacacagcttccctgactattacgggcgcgcaagccgaggatgaagcggactattactgcaactcccgcgacagctccggttacccggttttcggcactggtaccaaggtgaccgtgctgatcgaggtcatgtacccaccgccatatctggacaacgagaagtccaatggcacgattatccacgtcaaaggcaagcatctgtgcccctcccctcttttcccaggaccttccaaacccttctgggttcttgtcgtggtcgggggcgtgctcgcctgctactcattgctggttacagtggcattcattatcttttgggttcgtctgaagatccaggtgcgcaaagcggccatcaccagttatgagaagtccgacggagtttacacaggcctgtctaccaggaaccaggagacctacgagaccctgaagcacgagaagcctccccagtaa

SEQ ID NO: 40 - DNA, 2205 bp, synthetic construct
  source 1..2205: mol_type = other DNA
atggcactgccggtgacagccctgctcctgccgctggccctgctccttcatgcagcccgtccacaggttcaacttgtccagagcggtggcggactcgtccagcccggcaggtccctgagactgtcctgcgctgcctctggatttaccttcgatgactacgctatgcattgggtccgccaggccccagggaagggcctcgaatgggtctccggcatctcctggaactccggctccattggttacgccgacagcgtgaagggcaggtttactattagtagagataacgccaagaactctctctatctccagatgaacagtctgcgcgcagaagatactgcagtgtattactgcgctaaggacggcagcagttcctggagctggggctatttcgattactggggacagggcactcttgtgacagtatccagcggtgggggcggaagtggcggaggcggatccgggggcgggggtagctcctctgagctgacccaggacccggccgtctccgtagctctgggccagaccgtaaggactacctgtcagggggacgcactgcgctcctactatgccagctggtatcaacagaagcccggccaagcccccatgctggtgatctatggcaaaaacaataggccgagcgggatcccagataggttcagcggttccgacagcggagatactgcttccttgactatcaccggcgcgcaggccgaggacgaagctgactactattgtaatagccgggacagtagcggttaccctgtgttcgggactggcaccaaggtgaccgttctgaccactacgcccgcgcctaggcccccaacaccagcccctactatcgcctctcagccgctgtctctcaggcctgaagcgtgtaggccggctgccggcggggctgtccacactcgcggccttgatttcgcttgcgatatctatatctgggccccgctggcggggacatgcggggtcttgcttctgagcctcgtcatcaccttgtactgccgtgtgaaattttcccgctctgctgacgcccccgcctaccaacagggccagaaccaactgtataatgagctgaacctgggccgcagagaagagtatgatgtcctggacaagcgtcgcggccgggatccggagatgggcggtaagccccgtagaaagaacccccaggagggtctgtataacgagctgcagaaggataagatggctgaggcgtacagcgaaattggcatgaagggagagcggcgcagagggaaaggtcatgacggcctgtatcaaggcctctccaccgctactaaagatacctatgatgctttgcatatgcaggcgctgcccccacgtcgcgccaagagatccggctccggcgccaccaatttctctcttctcaaacaggccggagacgtggaagagaaccccggccctatgattgaaacgtataatcagacgtctccccggagcgctgccaccggtttgcccatttctatgaagattttcatgtacttgctgacggtctttttgatcactcaaatgatcggttctgctctgttcgcagtatacctgcatcggcgcctggataaaattgaggacgagcggaacctgcacgaagatttcgtcttcatgaaaaccattcagcgctgtaacactggtgagaggtctttgtccctcctgaattgcgaggaaatcaaatcccagtttgagggcttcgtgaaagacatcatgctgaacaaagaggaaaccaaaaaggagaatagttttgaaatgcagaagggagatcaaaatccccagattgctgcccatgtcatcagtgaggccagctctaaaaccacatccgtcctgcagtgggccgagaagggttattacactatgagtaacaatttggtgaccctggagaacggcaagcagctgactgtcaagcgccagggcctgtactatatctacgctcaggttacgttctgctccaatcgtgaggcctctagccaagcaccgtttattgcatccctgtgtctgaagtcccccggtcgtttcgagcgcatcctcctgcgcgctgccaacacccactcatccgccaagccctgtggacaacagtctatccatcttgggggcgtgtttgagctccagcccggcgcctctgtgttcgtcaatgttactgacccctctcaggtgagccatggcaccggttttaccagttttggtcttctgaagctgtaa

SEQ ID NO: 41 - DNA, 2196 bp, synthetic construct
  source 1..2196: mol_type = other DNA
atggccctgcctgtgaccgcgttgctcctgcccctcgccctgttgcttcatgccgctcgtccccaggtgcagctcgtccagagcggcggaggtctggtgcaacccggaaggagcctgcgcctgagctgtgctgcctccggtttcactttcgatgactatgccatgcactgggtcaggcaggctccgggcaagggtctggaatgggtctctgggatcagctggaactcaggatccatcgggtacgctgattctgtcaaggggagattcactatttcacgcgataacgctaagaacagtctctacctgcagatgaactctctccgcgcagaggatactgctgtctattactgtgcgaaggacggaagttccagctggagctggggttattttgattattggggccagggcaccctggtgaccgtcagttccggaggcggaggctccggcggtggaggctccgggggcggaggcagctcctcagaactcacccaagaccctgcggtaagtgtcgcattgggccagaccgttcgtaccacttgccagggcgacgccctgcgtagctactatgcgagttggtaccaacagaagccaggccaggcccctatgctcgtgatctatggcaaaaataaccgtcctagcggtattccagaccggttttctggtagcgatagcggcgataccgcaagcttgactatcacaggcgcccaggccgaagacgaagccgattattactgtaacagtcgcgactcatccggctatcctgtctttggcacgggtactaaagtcactgtgcttatcgaggttatgtacccgccaccgtatctggacaacgagaagagcaacggtacgattatccacgttaagggcaagcacctctgccccagtcctctgttcccaggaccatccaagcccttctgggtcctggtcgttgtgggaggggtccttgcatgctacagtcttctggtgacagtggccttcattatcttttgggtgcgtgtgaagttcagccgcagtgctgacgcccccgcatatcaacagggccaaaatcagctgtacaatgagctgaacctcggaaggcgcgaagagtacgacgtgctggacaagagacgcgggcgcgaccctgagatgggcggtaagcctagacgcaagaacccccaggaaggactctacaacgaactgcaaaaggataagatggctgaggcctacagcgagatcggcatgaagggcgaacggagacgcggcaagggtcatgacggcctttaccaagggctgtcaacagcgacaaaagatacgtacgacgcattgcacatgcaggccctgccacctcgcagggctaaacgcagtggcagcggtgccaccaattttagccttctgaagcaggccggcgatgtggaggaaaaccctggacccatgatcgaaacatataaccagacatcccccaggtctgccgcaaccggtctgcccatcagcatgaagatttttatgtacctgcttaccgtgtttttgatcacacagatgatcggcagcgctctcttcgccgtttacctccaccggcgtctggacaagattgaggacgagcgcaaccttcacgaggatttcgtctttatgaagactattcagaggtgcaatactggcgagcgttccctctccctgctcaactgcgaagagatcaagtcccaattcgaaggctttgtaaaggatatcatgctgaacaaggaagagacgaagaaagagaacagctttgagatgcagaaaggtgatcagaaccctcagatcgctgcccacgtcattagcgaggccagttccaagacaacctccgtcttgcagtgggccgagaagggttattacaccatgtccaataacctggtgacgctggaaaacggcaagcagctgacagtgaagcgccaggggctgtactatatctatgctcaggtgactttctgctctaacagagaggcatcctctcaggccccatttatcgcaagcctgtgcctgaagtcccccggccggtttgaaaggatccttctccgcgccgcaaacacccattccagcgctaaaccatgtggccaacagtctatccacctgggaggcgtcttcgagctgcagcctggcgcatctgtgtttgtcaacgtgaccgatccctctcaggtttcccatggcaccggcttcaccagttttgggctcttgaaactttaa

SEQ ID NO: 42 - DNA, 1848 bp, synthetic construct
  source 1..1848: mol_type = other DNA
atggccctgccggtcactgccctgctcctgcccctggccttgctcctgcacgccgctcgcccacaggtacagcttgtgcagagcggcggtggcctcgtccagccgggccgttctctccgcctgtcatgcgcagcttccggctttacgttcgacgattatgctatgcattgggtgaggcaggccccgggcaagggtctggagtgggtgagcggcatctcctggaatagcggttccatcggctatgcggattccgtcaaaggccgctttacaatctctagggataacgcaaaaaattccctgtacctccagatgaacagcctccgtgctgaggataccgctgtgtactattgcgctaaggacggttctagctcctggtcctggggctacttcgactactggggccagggcactctggtcaccgtctcttccggagggggtggcagcggtggaggcggaagcggaggcggaggctctagcagtgagctgacccaggaccctgctgtgtctgtcgccctgggccagacagtcagaaccacttgccagggtgatgctctccgcagctattacgcgtcctggtaccaacagaagcccggacaggcccctatgcttgtgatttatggaaagaataaccggcctagtggtatccccgataggttctcaggatccgactccggtgacaccgccagcctgaccatcactggagcacaggccgaggatgaagccgattattactgcaactcccgcgacagctccggttacccagtgtttggaaccggaaccaaagtgaccgtgctcatcgaggtgatgtacccgcccccgtacctggataacgaaaagagcaacggaaccattatccacgtgaagggaaaacacctgtgccccagccctctcttcccagggccaagtaagcctttctgggtgctggtagtggttggcggtgtactggcctgctactccctcctggtgaccgttgccttcattatcttctgggtcatggctgcaggaggcccgggagctggctccgcagccccggtatctagcacttccagcctcccccttgccgcgctcaacatgcgtgtgcggaggcgcctctccctgttcttgaacgtgcgtacgcaagtggctgccgattggactgcgctggccgaggaaatggatttcgagtatctggagattaggcagctggaaacccaggctgatcccacagggaggctcctggacgcctggcagggtcgccccggtgcaagtgtgggccgtcttctggagttgctgacaaagctgggacgcgatgacgtgcttttggagttgggcccgtccattgaagaggactgccagaagtacatcttgaaacagcaacaggaagaggcagagaaacctttgcaggtcgccgcggttgacagtagcgtgcctcgcaccgccgagcttgctggcatcacgaccctggatgacccgctgggccatatgcccgagcgttttgacgcgttcatctgctactgccccagcgacattcgtgtgaaattctcacgctctgcagatgcgccagcttaccaacagggccagaaccagctgtacaacgagcttaacctgggacgtagagaggaatacgacgtcctggacaaaaggagagggcgcgatcccgagatgggtggcaagccccgtcgcaagaaccctcaagagggtctctacaatgagttgcaaaaggacaagatggcagaggcatactctgaaattggcatgaagggcgagaggcgtagaggaaagggtcacgacggcctctaccagggcctgtccaccgctaccaaggatacctacgatgccctgcacatgcaggctctccctccaaggtaa

SEQ ID NO: 43 - DNA, 2034 bp, synthetic construct
  source 1..2034: mol_type = other DNA
atggccttgcctgtgaccgcattgctgcttccactggctctcttgctccacgccgcgagacctcaggtgcagctggttcagagcggaggtgggctggtccaacccggtcgctctttgagactgtcctgtgctgcgtctggatttacattcgatgactatgcaatgcactgggttcgtcaggcccctggcaagggcctggagtgggtgtccggcatcagctggaactccggatccatcggatacgctgatagtgtaaaaggccgtttcaccatctcccgcgacaacgcaaagaatagtctgtatctccagatgaatagcctccgcgctgaggataccgctgtgtactattgcgccaaagacggttcctctagctggtcttggggctactttgattactggggccaggggacactggtgaccgtcagctccggcggtggcggtagcgggggtggcggatccggtggcggtggctccagttccgagctgacccaggatccggccgtttctgttgccctggggcagactgtgaggacaacctgtcagggggacgctctgagatcttattacgcctcttggtatcaacagaaacccggccaggcccctatgctggtgatctacggaaagaataaccgcccatcaggcatccctgaccgtttctccggcagcgactcaggcgacactgcttccttgaccatcaccggagcgcaagctgaggatgaagccgactactattgcaactcccgcgacagctccggttatcctgtcttcggaaccggcactaaggtgaccgtgttgattgaggtgatgtacccgcccccatatctcgacaacgagaaatctaatggaaccattatccacgtgaagggcaagcacctctgcccgagccctcttttcccgggtccatcaaagccattctgggtgctggtggtagtcggcggtgtgctggcttgctactccctcctggtcactgtcgcgtttattatcttttgggtgatggccgctggaggcccaggcgccggtagcgctgcgccggtttcctcaacatcaagccttcctctggccgcactgaacatgcgtgtgaggcgcagactctctctctttttgaacgtgaggactcaggtcgccgctgactggaccgccctggccgaagagatggacttcgaataccttgaaatccgccagctcgagacccaagccgaccccactggtaggttgcttgacgcctggcaagggcgccccggcgctagcgtgggcaggcttctggagttgctgacaaagctgggccgcgacgatgtgctcctggagctgggcccctccatcgaggaagactgccagaaatacatcctcaagcaacagcaagaagaggctgagaagcctctgcaagtggccgctgtcgacagctccgtgccacgcacggctgagcttgctggcatcacaacccttgacgatcctctgggccatatgccagagaggttcgacgcgttcatttgttactgcccatccgacattaaaaaggtcgccaagaaacccaccaacaaggcacctcatccgaagcaggagccacaggagattaatttccctgatgacctgcccgggtccaatactgccgcaccggtgcaggaaaccttgcatggttgtcagcccgtcacccaggaggatggcaaggagtcccggatttctgtgcaggagcgccagcgcgttaagttctcccgcagcgccgacgcccctgcctaccaacagggccaaaaccagctctacaatgagcttaacctgggacgccgtgaagagtacgacgtgttggacaaacgccggggtcgcgaccctgaaatgggcggaaagcctaggcgtaagaaccctcaggaaggtctgtacaatgaactgcagaaggataagatggccgaagcctattctgagatcggaatgaaaggagaacgccggcgcggcaagggccatgacggcctgtatcagggcttgtccacggctactaaagacacctacgacgcattgcacatgcaggcgctgcccccacgctaa

SEQ ID NO: 44 - DNA, 2043 bp, synthetic construct
  source 1..2043: mol_type = other DNA
atggcgctgcccgtcacagccctcctgcttcccctggctctcttgctgcacgccgctagaccccaggttcagttggtgcagtctggaggcggtctcgttcaacctgggcgctccctgaggctcagttgtgcggccagcggcttcactttcgatgactacgctatgcactgggtgcgccaggccccaggcaagggcctggagtgggtctctggtatcagctggaactccggcagcattggctacgccgactctgtgaaagggcgcttcacaatcagccgcgacaacgcaaaaaactccctgtacctgcagatgaatagccttagagctgaggacacagccgtgtattactgcgccaaagacggaagctcctcttggagctggggttactttgattattggggccagggtaccctggtgaccgtgtcttcaggcggtgggggcagcggtggaggcggttctgggggcggaggctcctctagcgagctgacccaagaccccgccgtgtccgtggcccttggacagaccgtgcgtacgacctgccaaggcgatgcgctgcgcagctattacgcgtcttggtatcaacagaagcctggccaggcccccatgctggtcatctacggcaagaacaatcgcccaagcgggatccccgatcgcttctccggctcagacagtggagacactgcatccctgactatcacgggcgcccaggcagaagacgaggccgactattactgcaacagcagggacagctccggctatcccgtttttgggactggtacaaaagttacagtgcttactaccactcccgcacctaggcccccgaccccagcacctaccatcgcgtctcaacctctgtccctgcggccagaggcatgtcgtcccgctgcgggtggcgcagtgcatacccgcggactggatttcgcttgcgacatctatatttgggcgccccttgccggcacctgtggcgttctgctcctgtcactggtcatcactctgtactgtatggcagccggaggcccgggcgctggctctgctgccccagtgagctccactagctcattgcctctggctgcactgaacatgcgtgtgcgccggcgtctgagtctcttccttaacgtgcgcactcaggttgctgccgactggaccgccctggcggaagagatggacttcgaatatctggagatccggcagctcgagacccaggccgacccaacaggacgtctcctggacgcctggcagggacgccctggcgccagcgtgggtcgtctgctcgagcttctgactaagctgggtcgcgacgatgtcctgctcgagctcggcccgtctatcgaagaggactgtcagaagtacatcctcaagcagcaacaggaagaggcagaaaagcccctccaggtggctgccgtggattcaagcgtgccgcgtaccgcggagctggccggcatcactaccctggatgaccctctgggtcacatgcccgaacgtttcgacgctttcatctgctattgtccgagtgacatcaaaaaggttgccaaaaagccaaccaacaaggcgccgcaccccaagcaagagcctcaagagatcaacttcccggacgatctgcctggcagtaacacggccgcaccggtccaggaaaccctgcacggttgtcagcctgtcacccaggaggacggaaaggagtcccgcatcagtgtgcaggagcggcagcgcgtgaagttcagccgctccgccgacgcgccagcctaccaacagggtcagaaccagctgtataacgagttgaacctgggcaggagagaagagtatgatgtgctggacaagagaaggggccgcgaccctgagatgggcggtaagcctcgccgtaagaacccacaagagggcctgtacaacgagctgcagaaggataagatggccgaggcttactctgagattggtatgaagggcgagcgcaggcgcggcaagggccatgatggcttgtaccagggcttgtcaaccgctaccaaggatacctacgacgctttgcatatgcaggctctgcctccccgctga

SEQ ID NO: 45 - DNA, 1353 bp, synthetic construct
  source 1..1353: mol_type = other DNA
atggccctgcccgtgaccgcgctgctccttcccctcgcccttctcctgcacgcggccagacccggcagcgaagtccagctggtggagagcggcggaggcctggtgcagccgggaaggtccctgcgcttgtcatgtgcagcctccggcttcacattcgatgactacgccatgcactgggtgcgccaggccccgggaaagggactggagtgggtcagcggcatcagctggaattccggctctaccggctacgcggactctgttaaggggcgcttcacgatcagtcgcgacaatgcgaagaattccctgtacctgcagatgaacagtctgcgcgccgaagacaccgccctgtactattgtgccaaggattcatcctcatggtatggcgggggatccgcctttgatatttgggggcagggtactatggttaccgtctcctctgggggcggagggtctggagggggcggatctggcggtggcgggagctccagcgagctgactcaggaccccgccgttagcgtcgctttgggacagaccgtccggatcacctgccagggtgattccctgcgctcttattacgcctcctggtatcaacagaagcccggccaggcccctgtcctggtcatttacggcaagaataaccgtccaagtggcatccccgatcgcttttccggcagttcctctggcaacactgcaagtctcaccatcactggcgcccaggcagaggatgaagcagattattactgtaattctaggggctcctctggtaaccattacgtgttcggcaccgggaccaaggtaactgtgctgactacaacccccgcgcccaggccccctactcctgcgcccacaatcgcttcacagcctctcagcctgcgccccgaggcttgtaggcctgccgcgggcggtgcggtgcacacgcgcggcctggacttcgcttgcgatatctatatctgggctccactggctggcacctgtggggtccttttgctgagcctggtaattactctgtattgtcgcgtgaagttctcccgcagtgctgacgcccctgcctaccaacagggacagaaccagctgtacaatgagttgaacctcggacgtcgcgaagagtatgacgtgctcgacaagcgtcgcggccgcgaccctgagatgggaggtaaacctaggcgcaagaatccgcaggaaggcctgtacaacgaacttcagaaggataaaatggctgaggcctattccgaaattggcatgaagggagagcgccgtcgggggaaaggacacgatggactgtaccagggtctcagcaccgccaccaaggacacgtacgacgccctgcatatgcaggctctgcctccccgctaa

SEQ ID NO: 46 - DNA, 1830 bp, synthetic construct
  source 1..1830: mol_type = other DNA
atggcgctgcccgtgaccgcccttctgctccctctggccctgcttctgcacgccgctcgcccaggttctcaggttcaactggtccaaagcggagggggcctcgtgcagccaggccgttccttgcgtctgagctgcgccgcttccgggttcactttcgatgactacgccatgcactgggtgcgccaggcccccgggaagggactggagtgggtctccggcatctcatggaacagcggctcaatcggctacgctgacagcgtcaagggccgttttaccatctccagagacaacgccaagaattctctgtaccttcagatgaacagcctccgtgctgaggacacggcagtttattactgcgccaaggacggtagctctagctggtcatgggggtactttgactactggggccagggcaccctggtgactgtaagtagcggcggtggcgggtcaggtggcggtgggtctggagggggtgggagctccagcgagctcactcaggatcccgcagtgtccgtggccctggggcaaactgtgaggactacatgtcagggagacgccctccggagctattacgcctcctggtaccaacagaagcccggacaggctcccatgctggtgatttatgggaagaataacagaccctccgggattcccgacaggttctccgggtctgactccggcgatacggcttctctgacaatcaccggtgcccaggccgaggacgaggccgactactattgtaactcacgcgattctagcggatatccagtgttcggcaccggcactaaggtgacagtgctgattgaggtgatgtaccctcccccatacctggacaacgaaaagagcaacggcaccatcattcatgtcaagggcaagcatctgtgtcctagccccctgttccctggccctagcaagcctttctgggtgcttgtggtcgtgggcggagtgctggcttgttattccctgcttgttacagttgcattcattatcttttgggtgatggcggctggtggcccaggtgctggctccgctgccccagtgtcatccacctcttccctgccgctggcggccctgaacatgcgggtgcgccgtcggctgtctctgttcctgaacgttcgcacgcaggtcgccgctgactggactgcccttgcggaggaaatggactttgagtatctggaaatccgccagctggaaactcaggccgatccaactggccgtctgcttgacgcttggcagggtcgccctggcgctagcgtgggcagactcttggagttgctcacaaaactgggtcgtgatgacgtgttgctcgagctgggcccttccatcgaggaagactgtcagaagtatatccttaagcagcaacaggaagaggctgagaaaccgttgcaagtggctgcagttgactccagcgtaccgcgtaccgctgaattggccggcatcactacgctggatgacccactgggccacatgcctgagcgctttgacgctttcatctgttactgccctagcgacatcaaaaaggtggccaaaaagcctaccaacaaggcccctcatcccaagcaggaaccgcaagagatcaacttcccggacgatctgcccgggtctaacaccgccgcgcctgtgcaggaaaccctgcacggctgccagccagtgacgcaggaggacggcaaggagtccaggatctctgtccaggagaggcagaggctgaagatccaagtccgtaaagcggccatcacaagttatgagaaatccgacggtgtgtacaccgggctgagcacaagaaaccaggagacttatgaaaccctcaagcacgagaaaccccctcagtaa

SEQ ID NO: 47 - DNA, 1338 bp, synthetic construct
  source 1..1338: mol_type = other DNA
atggctctccctgtgaccgctctgcttctgcctcttgccctcctgttgcacgctgcccggcctgaagtccagctggttgagagcggtggaggcctggtgcagcccggacggtccctgcgcttgagctgtgctgcctccggcttcacctttgatgactacgctatgcactgggttcgccaggcccccggcaagggactggagtgggtgagcggcatttcctggaacagcggatccaccggctatgccgactcagtgaaaggtcgcttcacaatttccagagacaacgctaagaactccctttacctgcagatgaacagcttgcgggcggaggacactgccctctactattgtgctaaggatagctcatcctggtacggcggaggtagtgccttcgacatctggggacaaggtacgatggtaactgtgagttccggcggaggcggatcaggcggaggtggctctggcggtggcggatccagctccgagcttacccaggacccagccgtgagcgtggccctgggccagacggtccgcatcacgtgccaaggtgattcacttagaagctattacgcttcctggtaccaacagaagcccgggcaggcccccgtcttggtaatctacggcaagaataacagaccctctggtatccccgaccgcttttctggttcctctagcggtaacaccgcttccctgacaatcacaggcgctcaggccgaagacgaagcagattactattgcaacagccgcggctcaagtggcaaccactacgtctttggaaccggcactaaggtgaccgtactgattgaggtgatgtaccctcccccttacctggataacgaaaagagcaacggcactattatccacgtgaaagggaaacacctgtgcccttcacccctgttcccgggccctagcaaacctttctgggtgctggtggtcgtgggcggtgtgctggcctgttactccctgcttgtcaccgtagcttttattatcttttgggtccgcgtcaagttcagccgcagcgccgatgccccggcatatcaacagggccagaaccagttgtataacgaacttaacctgggacgtcgggaggaatacgacgttctggacaagcgccggggccgggatcccgagatgggtggaaagcctcgtcgcaagaacccgcaggaaggcctgtacaatgagttgcagaaagataagatggccgaggcttattctgaaatcggcatgaaaggtgaacgtagacgtggtaaaggccacgacggtctgtaccagggtctgagtaccgcgaccaaggacacatacgatgccctgcacatgcaggccctgccgccccgttag

SEQ ID NO: 48 - DNA, 2202 bp, synthetic construct
  source 1..2202: mol_type = other DNA
atggcgctgccagttacggcactgttgctccctctcgcgctgttgctccatgccgctaggcccgaggtgcagctggtcgagtctgggggcgggcttgtgcagcccgggaggtccctccgcctgagctgtgctgcctctggttttacctttgacgattacgccatgcactgggtcagacaggctcctggcaaaggcctcgagtgggttagcgggatctcttggaattctgggtccactggttacgccgactccgtcaagggccgtttcacgatttctcgcgataacgcgaagaattccttgtatctccagatgaattcccttcgcgctgaggacaccgctctgtactattgcgccaaggactctagttcctggtacgggggtggcagcgcgttcgacatctgggggcaaggcacaatggtcacagtcagctctggcggaggcggtagcggtggcggtggcagtggcggaggcggatcttcctctgagctgacgcaggacccggctgtttctgtcgctctcgggcagaccgtccgcatcacctgccagggcgacagtctgcgctcctattacgcgtcctggtaccaacagaagcctggtcaagccccggtcctggtgatctatggcaaaaataacagaccctccgggattccggatcggttcagtggttccagctctggcaatactgcttcactcactattaccggcgctcaggccgaagatgaggcagactattactgtaactctcggggaagctccggcaatcactacgtgtttggcacaggcaccaaggttaccgtgctgattgaggtgatgtatcctcccccatacctggacaatgagaagtccaacggaaccattatccacgtcaagggtaagcatctttgccctagtcccctcttcccaggcccttccaagcccttctgggtcctggtggttgtcggcggtgtgctggcctgttactccttgcttgtcacagtagctttcattatcttttgggtgagggtgaaatttagccgctctgcggacgctcccgcctatcaacagggccagaaccagctgtacaacgagctgaacctgggccgcagagaagagtacgacgtactcgataagaggcgcggccgcgatcccgaaatgggtggcaagccaagacgcaaaaaccctcaggagggcctgtacaatgagctccagaaggacaagatggctgaagcctatagcgagatcggtatgaaaggtgagcgccggcgcggtaagggccacgatggtctgtatcagggcctgtccacggctactaaggatacctacgacgcactgcatatgcaggccctgcctccgcggcgcgcaaagaggagtggcagcggtgccacgaatttttctctgcttaagcaggcaggcgacgtggaagagaacccaggccctatgatcgagacctacaaccagacctctcccagatcagccgctacggggctgcccattagcatgaagattttcatgtatctcctgactgtcttcctgatcacgcaaatgatcgggagcgccttgttcgcagtgtacctgcacaggcgcctggacaagatcgaagatgagcggaacctccacgaggatttcgtatttatgaagaccatccagcgctgtaacacaggggaacggtcccttagccttctgaactgtgaggaaatcaagtcccagtttgaaggatttgtgaaggacattatgctgaataaagaagagacaaaaaaggagaactcttttgagatgcagaagggcgaccagaaccctcagatcgctgcccatgtgatctcagaagcttcctcaaagaccacgtctgtgttgcaatgggccgaaaagggttattacacaatgagtaataacttggtcacacttgagaacggcaagcagctgacagtcaagagacagggcctgtattacatctacgcgcaagtgactttctgttccaacagggaagcctctagccaggccccgttcatcgcctctctgtgcctgaaatcccctgggagatttgaacgtatcctcctgcgggcagccaacactcacagctctgccaagccatgcggccaacagtccatccacctgggcggagtctttgaactccagcctggggcctctgtgttcgtcaacgtgaccgaccctagccaggtctctcatggcaccggcttcacttccttcggactcctgaaattgtga

SEQ ID NO: 49 - DNA, 1854 bp, synthetic construct
  source 1..1854: mol_type = other DNA
atggccctgcccgtgaccgccctgttgctgccgctggctctcctgttgcacgctgccaggcccgaggttcagctcgtggagtccggaggcgggctggtgcagcccggtcgctccctgcgcctctcatgcgccgcatctggttttactttcgatgactatgcaatgcattgggtacgtcaggctcctggcaagggtctggaatgggtgtccggcatcagctggaattccggtagcaccggttacgcagacagcgttaagggacggtttactatcagccgcgataacgccaagaactccctgtatctgcaaatgaactcccttcgtgccgaagacaccgctctttattactgcgccaaagacagtagctcctggtacggtggaggctccgcattcgatatctggggccagggcacgatggtaacagtgagcagtggcgggggcggatccggcggagggggtagtggaggtggaggcagttcatctgaactcacccaggatccggccgtgtccgtggctctgggccagactgtgcggatcacctgccagggggactctctccgctcttattacgcttcctggtatcagcaaaagccaggccaagcccctgtcctggtgatctatggtaagaataaccgccctagcggaattcccgataggttctccggatccagctccggcaacaccgcttccttgaccatcaccggggcacaggccgaggatgaggcagattactattgtaatagcagaggctccagtggcaaccactacgtctttgggaccggcaccaaggtgaccgttctcattgaggtcatgtatcccccgccctaccttgacaacgaaaagagcaacggaaccattatccatgtcaaaggcaaacacctgtgcccttccccactgttccctggcccttccaagcccttttgggtgctggttgtcgtgggtggcgtcctggcgtgttactccctcctggtaacagttgcgtttattatcttctgggttatggctgccggaggccccggggctggtagcgctgcccccgtaagtagcacatctagcctccccctggctgccctgaacatgagagtccggagacgccttagtttgtttctcaacgttcgcactcaggtggctgccgattggacagctctggccgaagagatggacttcgaataccttgagatcaggcagctggagactcaggctgaccccaccggcagactgctcgatgcctggcagggccgcccgggggcgtccgtcggccgcttgctggagctgctcaccaagctggggcgcgacgatgtgctcctggagctcggcccctccatcgaagaggactgtcagaagtatatcctgaagcaacagcaagaggaagctgaaaaaccactgcaagtggctgcagtggattcatctgtgccgagaaccgccgagctggctggtatcacgaccctcgatgaccctctcgggcacatgccagagaggtttgacgccttcatttgttactgtcccagcgatatccgcgtaaagttctcccgtagcgccgacgcaccggcctaccaacagggccagaatcagctctataacgaactgaacctggggcggagggaggaatacgatgtcctggataagcgtcgcgggcgcgatcccgagatgggtggcaagccacgtcgcaagaacccccaggagggtctctacaacgagctgcagaaggacaaaatggcggaggcctattctgagatcggcatgaagggcgagcgccggaggggtaaagggcacgacggtctgtaccagggcctgtctaccgctaccaaggatacctacgatgccctgcacatgcaagccctccctccccgctaa

SEQ ID NO: 50 - DNA, 2040 bp, synthetic construct
  source 1..2040: mol_type = other DNA
atggccctgcccgtgaccgccctccttctgccgctcgccctcttgctgcatgccgctcgcccagaggtccagctggtcgagtctgggggaggtctggtgcagccggggaggtctctgcgcctgtcatgtgccgcttcaggctttactttcgatgactacgctatgcactgggtacgtcaggcacccggcaagggcctggagtgggtttctggtatctcttggaatagcgggtctactggctacgctgacagcgtcaaggggcggtttactatcagccgcgacaatgctaagaacagcttgtacctccagatgaacagtctgagggccgaggacactgcactgtattactgcgctaaggattcatcttcctggtatggtggcggtagcgcatttgatatctggggtcagggcacaatggtgaccgtgagctctggtggcggtggatccggcgggggcggaagcgggggagggggcagttcttcagaactgacacaggaccctgccgtgagcgtagctttggggcagactgtccgcatcacctgccagggtgacagcctgcggtcctactatgccagctggtatcaacagaaacctgggcaggctcccgtgctggtgatctacggtaaaaataaccgcccgtccgggatccctgacaggttttccggcagttcctctggcaacacggcttctctgaccatcactggagcccaggcggaagacgaggctgattattactgcaactcccgcgggtctagcggcaaccactatgtcttcggcacaggcacgaaagtgactgtgcttatcgaggtaatgtatcctccaccctacctggacaacgaaaaatccaacggcacaattatccacgttaaaggcaagcacctgtgtccctcccccttgttccccggcccaagcaagcctttctgggtcttggtggtagttggtggcgtcctggcttgttacagcctgctcgttaccgtggccttcattatcttctgggtgatggcggccggaggccctggagctggctctgccgcgcccgtgtcttccacttccagcctcccgctggctgccctgaacatgagagtgcgtcgcagactgtcactgtttctgaacgtccgtacccaggttgctgccgactggacagcgctggccgaagagatggactttgagtacctggaaatccggcagctcgaaactcaggctgatcccacagggcgtctgctcgacgcgtggcagggaaggccaggcgcctctgtgggtcgcttgctggagctgcttactaaactgggcagggatgacgttcttctggagctgggcccgagcattgaggaagattgccagaagtacattctcaagcagcaacaggaggaagccgaaaagcccctgcaggtggccgctgtggattccagcgtgccgaggaccgccgagctggctggaatcactacccttgatgacccactcggtcatatgcccgagcgttttgatgcgttcatctgttattgtccgtccgatatcaagaaagtagcgaaaaagcctaccaataaagctccccacccgaaacaggagccgcaggagatcaactttcctgatgacttgcctgggagcaacacggctgcccccgtgcaggagactctccacggttgtcagcccgtgactcaggaagacgggaaggaaagccgtatctccgtgcaagagcgtcagcgcgtgaaatttagtagatctgccgacgctcccgcctaccaacagggtcagaaccagctgtacaacgagctgaacctcggccgccgtgaagagtacgatgttttggacaaaagacgcgggcgcgatcctgagatgggtggcaaacctcgtcgcaagaatccccaggaaggactgtataacgagctgcagaaggataaaatggcagaagcatacagtgagatcggcatgaagggcgaacgcaggcgcgggaagggtcacgacggactgtatcaggggctgtcaaccgccactaaagatacctacgatgctttgcatatgcaggccctgccccctagatag

SEQ ID NO: 51 - DNA, 2040 bp, synthetic construct
  source 1..2040: mol_type = other DNA
atggcgctgccggtgaccgccctgctcctgcctctggctctgttgctccatgctgcccgccctgaggtccagctggtggagagcggaggtggcctggtgcagcccggtcgcagcctgcgtctgagctgcgcggcttccggcttcaccttcgatgactatgccatgcactgggtccgccaggcccccggcaagggtcttgaatgggtgagcggaatcagttggaacagcggctcaaccggatatgcggattctgtgaaggggcgctttacaattagcagggacaatgcaaagaacagcctgtacctccaaatgaactccctccgcgccgaggatacagctctctattactgcgctaaggactcatccagctggtacggcggagggagcgctttcgatatctggggtcaggggaccatggtgactgtgtctagcgggggcggaggttccggtgggggcggttctggtggcggaggttccagtagcgagctcacgcaggaccccgctgtgagcgtggccttgggccagaccgtgcgtatcacttgccagggtgacagtctgcggagctattacgcctcctggtatcagcaaaaacccggccaggcccctgtgctggtaatctacggcaaaaataaccgcccttctggtatccccgaccgctttagcggctccagctccggcaatacagcctcactgaccattaccggagctcaggccgaggacgaggccgattactattgcaactccaggggaagctctggcaaccattacgtgttcgggacgggaaccaaggtgacagttcttattgaagtgatgtacccgccaccctatctcgataatgagaaatccaatgggacgatcattcacgtcaagggaaaacacctgtgcccaagccctttgttcccaggaccctccaaacccttctgggtgctggtggtcgtgggtggggtgctggcctgttattcattgctggttaccgttgctttcattatcttctgggtgatggctgccgggggccccggcgcaggcagtgccgctcctgtctccagcacctccagtctgcccttggccgcactcaacatgcgcgtccgtaggcgcctgagcctgtttctgaacgtacggactcaagtcgctgccgactggacagctctcgctgaagagatggatttcgaatacctcgagatccgccagctggagacccaggccgacccgaccggccgtctcctggacgcctggcagggtagacccggggcttctgtgggcaggttgctggagctcctgactaagctgggccgcgatgacgtgttgctggaactgggcccgtctatcgaggaagactgccagaagtacattttgaagcagcaacaggaggaagctgagaagcccctgcaggtcgctgccgtagactctagcgttccccggaccgccgaactggccggcattaccacactggacgatcccctgggtcacatgcctgagaggtttgacgcctttatctgctactgtccctctgatatcaaaaaggtggcaaaaaagcctactaacaaagctccacatcctaagcaggagccgcaggagatcaacttccctgacgatctgcccggttccaacactgccgctccggtgcaggagaccctgcatggctgtcagcctgttacccaggaggacgggaaggaatcccgcatctccgttcaggaacgccagcgtgtcaagttcagccgtagcgctgatgcccctgcgtatcaacagggtcagaatcagctctacaatgagttgaacctcggtcgtcgcgaggaatacgacgttcttgataagcggcgtggccgggaccctgaaatgggaggtaagccccgcagaaagaacccccaggagggcctctacaacgagctgcaaaaggacaagatggcagaggcctattccgaaattggcatgaagggggagcgcagacgcggcaaaggccacgatggtctgtatcagggtctgagtaccgcaactaaggacacctacgatgcactgcatatgcaggccctgccgccacgctag

SEQ ID NO: 52 - DNA, 1128 bp, synthetic construct
  source 1..1128: mol_type = other DNA
atggccctgcctgtgaccgccctgttgctgcctctcgctctcctgctccacgctgcgcggcctgaggtccaacttgtggaaagcggcggagggctggtccagccgggccgctccctgcgcctgagctgcgcagcctccggcttcaccttcgacgattacgccatgcattgggtgaggcaggccccaggcaagggcctggagtgggtgagtggcatcagttggaattccggctccacaggctacgctgactcagttaaggggagattcaccatttcccgcgataacgcaaagaacagcctgtacctgcaaatgaactccctgcgcgcggaggataccgcgctgtactattgtgcgaaggactcctcttcctggtatggaggcggtagtgcattcgacatctggggacagggaacaatggtgactgtgtccagcggcggaggtggctccggcggaggcgggtctgggggcggaggcagcagtagcgagttgacccaggatcctgctgtgagtgtggcgctgggtcagaccgtgaggattacatgccagggggactctctgcgctcctattacgccagttggtaccaacagaaacccgggcaggcgccggtcctggtcatctacggtaaaaataaccgcccgagcggcattcccgataggttcagcggtagttcctctgggaatacagcaagcctgactattaccggcgctcaggctgaagacgaagcagactactattgtaacagccgcggctccagcggcaatcattatgtgttcggaaccggtacaaaggtcaccgtactgattgaggtgatgtatccacctccctatctggataacgagaagtccaacggcactattatccacgtgaagggcaagcatctgtgtcctagcccactctttcccggcccctccaaacccttctgggtgctggtagttgtcgggggcgtgctcgcatgctattccctcctggtcaccgtggctttcatcattttctgggtgcggctgaaaatccaagtccgcaaagctgccatcacctcttacgaaaaatctgatggcgtctacaccggcctgtctacacgcaaccaggaaacctacgagacccttaagcacgaaaagccgcctcagtaa

SEQ ID NO: 53 - DNA, 732 bp, synthetic construct
  source 1..732: mol_type = other DNA
caagtgcagctacaacagagcggagccgaagttaaaaagcccggggcatccgtgaaggtttcatgcaaagcttccggctatactttcactggatattacatgcactgggtcaggcaggcgcccgggcagggactggagtggatgggccggattaatccaaattctggaggcactaactacgcccagaaattccagggcagagtgactatgacccgggacacaagtataagcactgcctatatggaactgtcgcgcctgcggtccgaagataccgcagtgtactattgtgccagggggcggtattacggaatggacgtctggggtcagggaactatggttaccgtgagctccggcggagggggttctggcgggggcggctccggcggtggcggatccggcgggggaggctctgaaattgttctgacccagtctcctgcaacccttagcctgagccccggcgaacgcgctacgatttcatgtagggcttcccagtcggttagtagcaactttgcctggtatcagcagaggccaggtcaggcccctaggcttctaatttacgatgcgagcaaccgagctacgggcattccaccaagattctctggtagcggaagtggcaccgattttacactgactatttcatcactcgaacccgaggattttgcagcctattactgccaccagaggtctaactggctgtacactttcggccagggaactaaggtggacataaaa

SEQ ID NO: 54 - DNA, 759 bp, synthetic construct
  source 1..759: mol_type = other DNA
caagtgcagctggttcagtcgggtgccgaagtgaaaaaacccggcgcttcagtgaaagtgtcctgtaaggcctccggatatacctttactggttactatatgcactgggtacggcaggccccaggtcaaggcctggaatggatgggatggattaaccctaacagcggcgggacaaattatgctcagaagtttcaaggaagagtgacgatgactcgcgacacctctataagtaccgcatacatggaactatcaaggctccgttccgacgatactgccgtgtactactgcgccagggacttgagaaggaccgttgtgacacctcgggcctattatggtatggacgtgtggggccagggcaccacagtgaccgtctctagcgggggcggcggatccggcggcgggggcagcggagggggtgggtctgggggaggtgggtccgatatccagctcacccagtctccatctaccttaagtgcctctgtcggggatcgcgtaacaatcacttgccaggccagccaggacattagcaattccttaaactggtatcagcagaaggccgggaaggcgcctaagcttctaatttatgatgccagcactctcgaaaccggggtgccgagtagattctccggatccggcagcgggaccgacttctctttcactattagttcgttgcagcccgaagatattgccacctattactgtcaacagcacgacaacttgcctctcacgttcggacagggaaccaaggttgaaattaaa

SEQ ID NO: 55 - DNA, 738 bp, synthetic construct
  source 1..738: mol_type = other DNA
caggttcagctggtccagagcggagccgaggtgaaaaagcctggcgcgccagtcaaagtctcatgtaaagcttccggatataccttcaccgggtactatatgcactgggtgagacaggccccagggcaaggcttagagtggatgggctggatcaacccaaactcggggggaacaaactatgcccaaaaattccaaggccgtgtcacaatgacacgtgacacttcaatatcaaccgcgtacatggaactttcacggctacgttccgatgacaccgcagtatactactgcgcaagaggggaatgggacggctcctattattacgattactggggacaaggcacgctggtgactgtatctagcggcgggggaggtagcggcggtggcgggagtggcggtggcggctccggaggagggggaagcgacatcgttctgacccagacaccttcttcgttgagtgcttctgtcggagacagggttacgattacctgtcgggcgtcacagtcaattaacacctatctgaactggtatcagcataagcctggcaaggctccaaagctgttgatctatgcagccagtagtcttcaatccggcgtgccatcccggttctcagggtcaggtagcggtactgattttactcttactatcagctccctccagcctgaggactttgccacatattactgtcagcagagctttagccctcttaccttcggggggggcaccaaactcgagattaag

SEQ ID NO: 56 - DNA, 726 bp, synthetic construct
  source 1..726: mol_type = other DNA
caggtgcagctggtggaatcaggtgggggtctagtgcagccaggaggctcactgagattaagttgcgctgctagtggcttcacctttagctcctactggatgcactgggtgagacaggtccccggcaagggcttagtgtgggtgtcaagaatcaacacagatggatctacaacaacatacgcagactccgtcgaagggcgcttcacaatctctcgtgacaacgcaaaaaacacgctttatctccagatgaactcactgcgagacgacgataccgccgtctactactgcgtgggaggccattgggcagtttgggggcaggggacaactgtcactgtctcttctggaggcggaggctcagggggcggcggcagtggcggtggggggagtggaggcggcggatcagatatccaaatgacacaatccccaagtaccttaagtgccagtgtcggcgaccgggtgaccattacatgccgagcctcccagtctattagcgatcgtttagcatggtatcagcagaagccggggaaggcaccgaagctgctcatttataaggctagtagcttagaatctggggttccatccagattttcaggttctggatcaggaaccgagttcactcttacgatctcctccctgcagccagacgacttcgctgtttactattgccaacagtacggtcatttgcctatgtacacatttgggcagggcaccaaggtcgaaatcaag

SEQ ID NO: 57 - DNA, 723 bp, synthetic construct
  source 1..723: mol_type = other DNA
caagtacaactcgttcagagcggggcggaagtcgagaaaccaggggcctccgttaaagttagctgcaaggcaagtggatacaccttcaccgactattacatgcattgggttcgacaggctcccggccaaggtctcgaatggatgggatggatcaatccaaacagcgggggcaccaactacgctcagaaatttcagggacgcgtgacaatgactagagacacgtccatttccacggcctacatggagctaagtcgactcaggtccgacgatacagccgtttattattgcgcctctggttgggacttcgattattgggggcagggtaccctagtgactgtgagttccggaggggggggatctggcgggggcggtagcggtggcggggggtcgggcggcggtggcagtgatatcgttatgactcagtcaccttccagccttagcgcaagcgtgggtgaccgcgtgacaattacctgccgggcatcccagtccatcagatattacttaagttggtaccagcagaagccggggaaggccccaaaactgctgatctacaccgccagtatcctgcagaatggggtgccatccagatttagtggctccggatccggcactgatttcactctgacgattagctctctgcaacctgaggattttgcaacctattactgcctgcagacctataccacccccgattttgggcccgggaccaaagtggagatcaaa

SEQ ID NO: 58 - DNA, 759 bp, synthetic construct
  source 1..759: mol_type = other DNA
caggtgcaactcgtgcaatcgggagctgaagtgaagaagccaggggccagcgttaaagtgtcctgtaaggcaagtggttacacatttacaagctactatatgcattgggtccgacaggctcctggacaggggttggagtggatgggaatcatcaatccgagtggcggcagcacttcctatgcccaaaagttccagggcagggtgacaatgacccgggacacgagtactagtacagtctatatggagctgagctccctcagatcagaggataccgcagtgtactattgtgcacggtatcggctgatcgccgttgccggtgactattactattacggcatggatgtgtggggccaggggacgatggtgaccgtgtctagcggcggcggggggagcggtgggggagggtccggcggcggtggaagcggcgggggcggatcagacatccaaatgacacagtcaccctcctcggtgagtgcctccgtgggagaccgtgtgactatcacttgtcgcgccagccagggggtgggtcgatggctggcgtggtatcagcagaaaccaggcaccgctcctaagcttcttatctacgcagcatcgactctccagagcggcgtcccaagccggttcagtggctccgggagcgggaccgactttacccttaccataaacaatctccagccagaagacttcgcaacctactattgccagcaggctaattcctttccgttgacttttggcggggggacacgcctggagattaag

SEQ ID NO: 59 - DNA, 750 bp, synthetic construct
  source 1..750: mol_type = other DNA
caggtgcaactggtccagagcggaggaggcgtggttcagcctggaaggtctctgcgattgtcttgcgcagcgagcggcttcaccttctcttcatacgcaatgcactgggttcgccaggcacccggcaaggggctggagtgggtcgcagtaattagttatgacggctctaataaatattatgctgactctgttaagggcagattcacaatctctcgcgacaacagtaaaaacacactatatctgcagatgaacagcttgcgtgccgaagacacagcggtttactattgtgccagatggaaggtttctagctcctctccagccttcgattattggggtcagggcacactggtcacggtctcaagcggtgggggtggctcaggcggcggcgggtccggtggcggcggatcaggagggggcgggagcgaaattgtgttgacccaaagtccagccaccctgtccctgtccccaggagagcgagcgatcttaagctgtagagcttctcaatccgtctacaccaagtacttaggctggtatcagcagaaacctggtcaggctcctaggttgctgatttacgacgccagtacgcgcgcaacgggcattcccgaccggtttagcggcagcggtagcggaactgattttaccctcaccatcaaccgtttggaaccagaagacttcgccgtgtactattgccagcactatggcgggtctcccttaatcaccttcggccaggggacccggctggaaatcaag

SEQ ID NO: 60 - DNA, 738 bp, synthetic construct
  source 1..738: mol_type = other DNA
caggtgcagctgcagcagtcaggcgcagaggtgaagaaacccggagctagcgttaaggtaagctgcaagacctccgggtacccctttacagggtacagtctgcactgggttcgacaggcaccagggcagggcttggaatggatggggtggatcaatcctaactcaggcggcaccaactacgcccaaaagttccaggggagagtgacaatgaccagggatacaagcattagcactgcatatatggagctatcacggctgcggtccgatgacactgcggtgtactactgcgctcgtgaccactatggaggcaacagcttgttctactggggtcagggaaccctcgtgacagttagctccggagggggcgggtctggcggcgggggatctggaggtggtggatccggaggtggcggaagcgacattcagctcacgcagtcgcccagctccatcagcgcctctgttggagacaccgtgagtattacctgtagagcaagccaagacagtggcacctggttggcgtggtaccagcaaaaacctggtaaggctcctaatttactcatgtacgatgcttccacactggaggacggagtgccgtcccggtttagcgggagcgcctcaggaacagaatttacattaaccgtcaataggctgcagcccgaagactcagcaacttactattgtcaacagtataacagctatcctctaacttttggaggtggcacaaaggtagatataaag

SEQ ID NO: 61 - DNA, 744 bp, synthetic construct
  source 1..744: mol_type = other DNA
caggtgcagctagtgcaatccggcgctgaggttaaaaagccaggtgccagcgttgaggtgagctgtaaggctagcggctacacttttacttcatattacatgcactgggttcgtcaagcccctggccagggactggagtggatgggaattatcaacccttccgggggctctactggctacgcacaaaaattccagggccgcgtgaccatgacacgggacacttcaaccagcaccgtgcatatggagctgagctcactcaggagcgaagacaccgctgtgtactattgtgctagaggcggctactctagctcctctgacgccttcgacatctggggtcagggtactatggtgacagtaagcagcggcggcggtgggtcaggaggggggggatctggaggcggtggcagcggtggcggagggtccgacattcagatgactcagtcacctccctccttgagcgcttctgtgggcgatagggtaaccatcacatgccgagcaagccaggacatcagttccgcacttgcgtggtaccagcagaagccgggcactcctcccaagctcctaatttatgatgcttcttcattagaatcaggcgttccttcacgcttcagcggtagcggctcaggtaccgatttcactctcaccatttctagtctacaaccagaagattttgcaacttattattgccagcagtttagctcttatcctttaaccttcggtggaggtactcgactcgagataaaa

SEQ ID NO: 62 - DNA, 765 bp, synthetic construct
  source 1..765: mol_type = other DNA
caggtccagctggtccaaagcggtgcagaagttaagaaacctggtgcctccgttaaagtatcttgcaaagccagcggatacacttttacgtcatatgggatctcttgggttagacaggctccagggcaggggttggaatggatgggttggatctcagcgtataacgggaacactaattacgcgcagaagctccagggcagggtcactatgactactgatacaagcacaagcaccgcttacatggaactaagaagcctgcggtccgatgacacggcagtttattactgcgcaagggttgcaggcggaatttactattactacggcatggatgtttggggccaagggactactattaccgttagcagcggaggaggcgggtcagggggaggcggttcgggcggcggaggcagtgggggcggcggttccgatattgtcatgacacagacaccggactccttggccgtttcattgggcgaacgcgcgactatcagctgtaagagcagtcatagcgttctctacaacaggaacaacaagaactacctcgcgtggtatcagcagaaacctggccaaccacctaaactgctgttttactgggcatccacaagaaagtcgggtgtgccagaccgattctcgggttctgggtccggcacggacttcacgctgacaataagctcactccaacctgaagatttcgcaacctatttctgtcagcagacccagactttccccctgacatttgggcagggcacgaggttggagatcaat

SEQ ID NO: 63 - DNA, 723 bp, synthetic construct
  source 1..723: mol_type = other DNA
caggtccagctgcagcagtccggagcagaagtcaagaagcctggcgcctcagtcaaagtgtcatgcaaggccagcggctatacattcaccggatactacatgcattgggtcaggcaggcacctgggcaggggctggagtggatgggctggattaatcctaactcagggggcaccaactatgcccagaactttcagggacgcgtgacaatgactcgagatacttcaatttctacagcctacatggagctccggaggctgagatccgacgacacagcggtgtactactgcgccagtggatgggactttgactattgggggcagggtactctggtgacggtgtcctctgggggtggcggcagcggcggggggggcagcggaggcggggggagtgggggcggaggatcggacattaggatgacacagagcccaagctccctctctgccagcgtaggcgacagggtcactattacatgccgtgcctcccagtctattaggtactatctgtcctggtatcagcagaaacctggaaaagcgcccaagctcctgatctacacagcctcgatcctccaaaatggggtgccatcgagatttagtgggtccggttctggcactgactttaccctgactatctcgtcacttcagcctgaggactttgcgacttattactgccttcagacatatactactcccgacttcggtccaggcaccaaggtagagataaag

SEQ ID NO: 64 - DNA, 738 bp, synthetic construct
  source 1..738: mol_type = other DNA
caggtgcaattggttcagagcggggccgaagtcaagaagcctggggcctccgtgaaggttagctgcaaagccagcggatacacctttacaggctactatatgcactgggttcgacaggcccccggtcagggacttgaatggatgggaaggatcaaccccaacagcggcggaactaattacgcccagaaattccaggggagggtgacaatgaccaccgacacctcaacttccacagcatacatggagttgagatccctgagatctgacgacaccgcagtttattactgtgctagaaccacaacctcctacgccttcgacatttggggtcaaggaaccatggtgaccgtgtcttctggcggtggcgggagcggcggaggtgggtcaggaggagggggaagtggagggggcggttcagatattcagctcacacaatccccctctacattgagcgcatctgtaggcgaccgtgttacaataacgtgtcgcgcgtcccaatccatcagtacatggttggcctggtaccagcagaagccgggtaaagctcctaatttgctgatctataaggcctccactctggagagcggcgtgcctagccggtttagtgggtctggatcggggaccgagtttaccttaactatcagtagcctacagccagacgattttgctacatactactgccagcagtacaatacatacagcccttacaccttcggccaaggcactaagctggagataaag

SEQ ID NO: 65 - DNA, 747 bp, synthetic construct
  source 1..747: mol_type = other DNA
caagtgcagctggttcagtcaggcggtgggctcgtgaaaccagggggcagtctacgtctgagttgcgaggcaagcggctttatcttttccgattattacatgggttggatcagacaggccccaggcaaagggctcgagtgggtgagctacatcggccgttcaggtagttctatgtactatgctgacagtgtgaaagggcggtttacttttagccgggacaacgcaaagaactctctgtacctgcaaatgaactcgttgcgagcggaggacacagctgtatactattgcgccgcatcacctgttgtggccgcaacggaagacttccagcactgggggcagggaacccttgtcacggtatctagcgggggtggtggctcaggcgggggcggcagtggcggcggagggtcagggggcgggggatccgacattgtgatgacacaaactcccgcgactttgagtctgagcccaggagaacgtgcgaccctctcctgtagggcctcacagagtgtgaccagtaattacctagcctggtatcagcagaagccgggacaagctccgcgtttgctgctatttggcgcgtccactcgggcaaccggcatcccagaccggttctcaggatccggtagtggaactgatttcacgctgaccattaatcggctcgaacccgaggatttcgccatgtattattgtcagcagtacggctctgcacccgttacatttggccaaggaaccaaacttgaaattaag

SEQ ID NO: 66 - DNA, 747 bp, synthetic construct
  source 1..747: mol_type = other DNA
caggtacagttggtgcagtccggcgcagaggtaagggcccctggcgcttctgtaaagatcagctgcaaggcttcaggatttacattccggggatactacattcactgggtgcgccaggctcctggccaaggcttggagtggatgggcatcatcaatcccagcggcggatctcgcgcctacgcacaaaagttccaaggccgcgtgaccatgacccgtgacacatcaacttccacagtctatatggaattaagcagtctccggtcagacgacaccgcaatgtactactgtgcccgaaccgcctcctgcggtggagattgttattatctggattattgggggcaaggcaccttggtgaccgtttcctcagggggcggcggatcgggggggggcggttccgggggaggaggaagcggcggcggtggctctgatatccagatgactcagagtccgcccactctgagcgcgtccgttggtgatcgcgttaccattacatgccgtgcctctgagaacgtgaacatttggttggcttggtaccagcagaagcccggaaaagcccccaaattgctgatctacaaaagcagcagcctggcctcaggagtaccaagcagattttccggctccggaagcggagcagaatttaccctgacaatctccagcctgcagcccgacgattttgccacatattactgccagcagtaccagtcctatccactcacttttggcggtgggactaaagtggatattaaa

SEQ ID NO: 67 - DNA, 732 bp, synthetic construct
  source 1..732: mol_type = other DNA
caggtgcagttggtgcaatctggcggaggcttggtccagccaggtagaagtttgaggctctcctgtgccgcttccggctttaccttcgatgactacgctatgcactgggtgagacaggccccgggaaaaggtctggaatgggtctccgggatcagctggaacagtggctctatcggttacgcggacagtgtaaaggggaggttcaccattagtcgcgataacgccaagaatagcctctatctgcagatgaactccctgagggccgaagacacagcagtgtattactgcgcgaaggatggcagcagcagctggagttgggggtacttcgactactggggccagggcactttggtcacagtctccagcggaggagggggaagtggcggtggaggaagcggcggcggcggttcttcgagcgaactgacgcaggatcctgcagtgtctgtagccctgggccagaccgttcggaccacgtgccaaggcgatgcgctcagatcttattacgcatcatggtaccagcaaaagccaggacaggctcctatgttggtgatctacgggaagaataaccgcccatccgggatcccagatcgcttctccggtagcgactcgggagatacagcctccctgaccataaccggcgcccaggcagaggacgaagctgattactactgtaattctcgcgactcgtccggataccccgttttcggtacgggcaccaaggtcacagtgctc

SEQ ID NO: 68 - DNA, 738 bp, synthetic construct
  source 1..738: mol_type = other DNA
gaggtgcagttggtcgaatctggcggcgggctagttcagcctgggagaagccttcgacttagctgcgccgctagtggctttacattcgacgactacgccatgcattgggttagacaagctcccggaaagggtcttgaatgggtctccggaatatcttggaattcgggatccacaggttacgcagattcagtcaaaggtcggtttactatatcccgggacaacgcaaagaatagcctgtaccttcagatgaactcactgagagcggaggacactgctttgtactactgtgccaaagattcctcatcttggtatggaggaggaagcgcgtttgatatatggggccagggaaccatggtcacggtatcttctggcggcggcggtagcggaggcggcggctccggcggaggaggatcgtcaagtgagctgacacaagagcccgcagtgtcagtggcccttggacagactgtcaggatcacttgccagggagattcactgagatcttattatgcaagctggtaccagcaaaaaccaggtcaggcaccagtactggttattttcgggcggagcaggcgcccttcaggcattcctgaccgtttttccgggtccagctccggtaacacagctagtctgatcatcacgggagcccaagctgaagatgaagccgattactactgcaactcgcgcgacaataccgctaatcactatgtgttcgggaccggcaccaaattaactgtcctg

SEQ ID NO: 69 - DNA, 738 bp, synthetic construct
  source 1..738: mol_type = other DNA
gaagtgcagctggttgagtctggaggcgggctggtacaacccggcaggtccctcagactttcatgcgcagccagtggattcacctttgacgactacgccatgcattgggtaaggcaggctccaggcaaaggtctggagtgggtgtcaggcatatcgtggaatagcgggagcaccggctatgcagacagtgttaagggaaggtttaccatatcacgggataatgctaaaaattcattgtatttacagatgaactccctgagggccgaggataccgccttatattactgcgcaaaggacagttcctcctggtatggtggggggagcgcattcgatatctggggccaaggcactatggtgacagtgtcctccgggggagggggttcaggagggggcgggagcggtgggggtgggtccagcagtgaacttactcaggaccccgccgtgtctgttgctctaggccaaacggtgagaataacctgtcaaggcgattccttgaggtcatactacgccagctggtaccagcagaaacctgggcaggcccctgttctagtgatatacgggaaaaataaccgtccttccggaatccctgatcgcttttctgggtcttcatctggtaacactgcctctctcacaattaccggagcacaggcagaggatgaggccgactactactgcaattctcggggatcctctggcaaccattatgtcttcggcacaggcacaaaagtgactgtgctt

SEQ ID NO: 70 - DNA, 753 bp, synthetic construct
  source 1..753: mol_type = other DNA
caggtgcagctggtgcagtcaggtgggggtttagtgcagcccggaggctccctgcggctctcctgtgcggcctcaggattcactttttcctcttactggatgcactgggtgcggcaagctcccggcaaggggctggtatgggtcagccggatcaattcagacgggtccagcacgagctacgccgactcagtcaagggaagatttaccatcagccgagataacgcaaaaaacactctttacctgcaaatgaattcattacgcgccgaggatacggccgtatactattgcgtgaggacaggctgggttggtagttactactactatatggacgtatggggcaagggtaccaccgttacagtgagctcaggaggtggaggctctggaggtggaggcagcggcggaggaggatcaggcggaggtggctccgagattgtcttgacccagtctcccgggactttgtcactgtctcccggtgaaagggccaccctgtcctgcagggctagtcaatccgtgagtagtaattacctggcatggtatcagcaaaagcctggccagccaccaaggctgctcatttatgatgtgtcgacacgagccacagggatacccgcacgattctcaggcggtggctccgggaccgactttactctgaccatcagcagtctagagccagaggatttcgctgtgtactactgccaacagcgcagtaactggcctccttggacgttcggtcagggaactaaggtggaaattaag

SEQ ID NO: 71 - DNA, 750 bp, synthetic construct
  source 1..750: mol_type = other DNA
caggtccagctagtccagtcgggcggaggagtcgtccaacctgggagaagtttacgactgtcctgcgcggcctcagggttcaccttctctagttacggcatgcactgggtaagacaggcacctgggaagggccttgagtgggtggcggtcatctcctatgatggcagtaacaaatattacgctgactctgtgaaaggtaggtttactatttccagagataattccaaaaacactctttatctacagatgaacagcctcagagcagaagataccgccgtatattattgcgcaaagggctactcaaggtactactactatgggatggacgtgtggggacaggggacgacagtgaccgtttcaagtggaggcggtgggtctgggggcggcgggagcggcgggggcgggtccggcggtggcggcagcgagatcgtgatgactcagtcacctgcaaccttgtcattgagccctggggagcgagcaattctgtcctgcagagcctcccagagcgtctacactaagtacctgggatggtaccagcagaaacccggccaggctcctcgcttactgatttacgacgcttccactcgagcaactggcatacccgatagattctcaggaagtgggagcggaactgatttcaccctgacaatcaatagacttgagcctgaggactttgctgtatattactgccagcattacggcgggtccccgctgatcacctttggccagggtacaaaggtcgacattaaa

SEQ ID NO: 72 - DNA, 747 bp, synthetic construct
  source 1..747: mol_type = other DNA
caagtgcagctcgtccagtcagggggtgggctggttcaaccggggggtagcttgcgtctgtcctgtgctgcttccggttttacattcagctcctatgccatgtcatgggtacgacaagctcccggcaagggactcgaatgggtaagtgcaatatctggatctgggggctctacctactacgccgattccgttaagggccggttcacaatcagtcgggacaatagtaagaatacactgtacctgcagatgaacagcctgcgagctgaggatacagcggtgtattactgtgctaaacgtgaggccgcggcaggacatgactggtactttgacttatggggccgaggaacactggtgaccgtctcttcaggagggggcggaagcggaggaggtggaagtggcgggggaggatccggaggcggaggctctgatattagggttacccagtcccccagctcactgagcgcgtcagtgggcgatcgcgtcacgataacttgtcgtgcttcccagagcattagtagctaccttaactggtaccagcaaaagcctgggaaggcacccaaattgttgatttatgccgcctcctccctacaatccggggttccttctaggtttagcggttctggttcggggaccgacttcactctgaccatatcaagtcttcagcccgaggacttcgccacctattattgccagcagtcctattcaatcccactgacattcggccagggcaccaaggtggaaataaaa

SEQ ID NO: 73-74 - skipped (2 entries)

SEQ ID NO: 75 - protein, 375 aa, synthetic construct; identical to SEQ ID NO: 3

SEQ ID NO: 76 - DNA, 1694 bp, synthetic construct
  source 1..1694: mol_type = other DNA
atggccctgcctgtgactgctttgctcctgccgttggcgctgctcctgcacgctgcccgtcctggttcccaggtgcagctggtccaaagcggcgggggtctcgtgcagccagggcgttctctgagactgtcttgcgctgcctctggtttcactttcgacgattatgctatgcattgggtccgccaggcgccagggaagggactggagtgggtttccgggatcagctggaactccggctcaatcgggtatgccgactcagttaagggcaggttcaccatctccagagacaacgccaagaattcactgtacctccagatgaacagcctgagggctgaggataccgccgtgtactattgtgccaaagatggatcttcctcttggagctggggctacttcgattactggggccaagggaccttggtaacagtctcatccgggggaggtggctctgggggcggtgggtccggcgggggcggttcttccagcgagcttacgcaagacccagccgtgtctgtggcactgggccagacagtgcgcaccacttgccagggagatgcgctgcgcagctattacgccagctggtaccaacagaagcctggccaggctccaatgctcgtcatttatggcaagaataaccgcccctccgggatcccagaccggttcagcgggagcgactcaggcgacaccgcttccctgactatcacaggcgcccaggctgaggacgaggcggactactattgcaacagtcgggacagctctggataccctgtgtttggcaccggcaccaaagtcaccgtgctcattgaggttatgtaccctcccccttatctcgacaacgagaagtctaacggaaccattatccatgttaagggcaagcacctgtgtcccagcccactgtttccaggccccagcaagccattctgggttctggtcgttgtgggcggggtgctcgcttgctactccttgctggtgactgttgcgttcattatcttctgggtcaggctcaagatccaggtgcgcaaggctgccatcactagttatgaaaagtctgacggtgtgtacactggcctctctacgcgcaaccaagagacgtatgagaccctgaagcacgaaaagccgccacagtaaaagttgtctcctcctgcactgactgactgatacaatcgatttctggatccgcaggtaagtgtgctcgcttcggcagcacatatactatgaattcaaagtcgactggaggcttgctttgggctgtatgctgtagatgttccgttcattagatcttttggcctctgactgagatctatgacggaacatctacaggacacaaggccctttatcagcactcacaatgtctagaaaacctggaggcttgctttgggctgtatgctgtgattgtagattaattcccggcttttggcctctgactgagctggggttatctacaatcacaggacacaaggccctttatcagcactcacattgcggccgcaacctggaggcttgctttgggctgtatgctgtaaactcctgaaaccatgctccttttggcctctgactgaggggtaggttcaggagtttacaggacacaaggccctttatcagcactcacatggaacaaatggccaccgtgggaggatgacaactcgagccttttcaattgtactaacttcgagtcttctttttttttttcacag

SEQ ID NO: 77 - RNA, 21 nt, synthetic construct
  source 1..21: mol_type = other RNA
attgtagattaattcccggcc

SEQ ID NO: 78 - RNA, 22 nt, synthetic construct
  source 1..22: mol_type = other RNA
tgattgtagattaattcccggc

SEQ ID NO: 79 - RNA, 21 nt, synthetic construct
  source 1..21: mol_type = other RNA
atacattcaccaggagccagc

SEQ ID NO: 80 - RNA, 21 nt, synthetic construct
  source 1..21: mol_type = other RNA
atgctggcatactccgtgtgg

SEQ ID NO: 81 - RNA, 22 nt, synthetic construct
  source 1..22: mol_type = other RNA
agatgttccgttcattagatcc

SEQ ID NO: 82 - RNA, 21 nt, synthetic construct
  source 1..21: mol_type = other RNA
ttccgttcattagatccagtg

SEQ ID NO: 83 - RNA, 21 nt, synthetic construct
  source 1..21: mol_type = other RNA
tagatccagtgttctcagcgg

SEQ ID NO: 84 - RNA, 22 nt, synthetic construct
  source 1..22: mol_type = other RNA
taaactcctgaaaccatgctcc

SEQ ID NO: 85 - RNA, 22 nt, synthetic construct
  source 1..22: mol_type = other RNA
tagatgttccgttcattagatc

SEQ ID NO: 86 - RNA, 22 nt, synthetic construct
  source 1..22: mol_type = other RNA
tgattgtagattaattcccggc

SEQ ID NO: 87 - RNA, 22 nt, synthetic construct
  source 1..22: mol_type = other RNA
tagatgttccgttcattagatc

SEQ ID NO: 88 - RNA, 22 nt, synthetic construct
  source 1..22: mol_type = other RNA
taaactcctgaaaccatgctcc

SEQ ID NO: 89 - RNA, 22 nt, synthetic construct
  source 1..22: mol_type = other RNA
ggaaagtcgacatagagccgct

SEQ ID NO: 90 - RNA, 22 nt, synthetic construct
  source 1..22: mol_type = other RNA
gatatatgctctcaagggtgct

SEQ ID NO: 91 - RNA, 22 nt, synthetic construct
  source 1..22: mol_type = other RNA
aacttgagttcttcctgcttcc

SEQ ID NO: 92 - RNA, 22 nt, synthetic construct
  source 1..22: mol_type = other RNA
ttaaacttgagttcttcctgct

SEQ ID NO: 93 - RNA, 22 nt, synthetic construct
  source 1..22: mol_type = other RNA
cttaaacttgagttcttcctgc

SEQ ID NO: 94 - RNA, 22 nt, synthetic construct
  source 1..22: mol_type = other RNA
ttagcaggagtttctatcaagc

SEQ ID NO: 95 - RNA, 22 nt, synthetic construct
  source 1..22: mol_type = other RNA
ttgttgatgatttctccagtgc

SEQ ID NO: 96 - RNA, 22 nt, synthetic construct
  source 1..22: mol_type = other RNA
atttttgagctgagcaagatcc

SEQ ID NO: 97 - RNA, 22 nt, synthetic construct
  source 1..22: mol_type = other RNA
aaatgtgtctgtatgttcctgc

SEQ ID NO: 98 - RNA, 22 nt, synthetic construct
  source 1..22: mol_type = other RNA
tatcctaggtacatacacgttc

SEQ ID NO: 99 - RNA, 25 nt, synthetic construct
  source 1..25: mol_type = other RNA
tggggaaagtcgacatagagccgct

SEQ ID NO: 100 - RNA, 25 nt, synthetic construct
  source 1..25: mol_type = other RNA
tctgatatatgctctcaagggtgct

SEQ ID NO: 101 - RNA, 25 nt, synthetic construct
  source 1..25: mol_type = other RNA
ttaaacttgagttcttcctgcttcc

SEQ ID NO: 102 - RNA, 25 nt, synthetic construct
  source 1..25: mol_type = other RNA
gtcttaaacttgagttcttcctgct

SEQ ID NO: 103 - RNA, 25 nt, synthetic construct
  source 1..25: mol_type = other RNA
tgtcttaaacttgagttcttcctgc

SEQ ID NO: 104 - RNA, 25 nt, synthetic construct
  source 1..25: mol_type = other RNA
ccattagcaggagtttctatcaagc

SEQ ID NO: 105 - RNA, 25 nt, synthetic construct
  source 1..25: mol_type = other RNA
gtgttgttgatgatttctccagtgc

SEQ ID NO: 106 - RNA, 25 nt, synthetic construct
  source 1..25: mol_type = other RNA
gagatttttgagctgagcaagatcc

SEQ ID NO: 107 - RNA, 25 nt, synthetic construct
  source 1..25: mol_type = other RNA
tgtaaatgtgtctgtatgttcctgc

SEQ ID NO: 108 - RNA, 25 nt, synthetic construct
  source 1..25: mol_type = other RNA
acatatcctaggtacatacacgttc

SEQ ID NO: 109 - RNA, 22 nt, synthetic construct
  source 1..22: mol_type = other RNA
gtttaaaccgagaatattctcc

SEQ ID NO: 110 - RNA, 22 nt, synthetic construct
  source 1..22: mol_type = other RNA
caaatagactccatgattcgta

SEQ ID NO: 111 - RNA, 22 nt, synthetic construct
  source 1..22: mol_type = other RNA
gtaattcccaagaatgtgcccg

SEQ ID NO: 112 - RNA, 22 nt, synthetic construct
  source 1..22: mol_type = other RNA
aaataacttgggaactgacgtc

SEQ ID NO: 113 - RNA, 22 nt, synthetic construct
  source 1..22: mol_type = other RNA
tttaaaccatagaacactaact

SEQ ID NO: 114 - RNA, 22 nt, synthetic construct
  source 1..22: mol_type = other RNA
actaaatagcttaggatatgct

SEQ ID NO: 115 - RNA, 22 nt, synthetic construct
  source 1..22: mol_type = other RNA
attttgatactgaactcaagcc

SEQ ID NO: 116 - RNA, 22 nt, synthetic construct
  source 1..22: mol_type = other RNA
taatagagaagctgaactttgc

SEQ ID NO: 117 - RNA, 22 nt, synthetic construct
  source 1..22: mol_type = other RNA
aattataacctgggaatggagc

SEQ ID NO: 118 - RNA, 22 nt, synthetic construct
  source 1..22: mol_type = other RNA
ttcataaccctggaacaagacc

SEQ ID NO: 119 - RNA, 25 nt, synthetic construct
  source 1..25: mol_type = other RNA
tctgtttaaaccgagaatattctcc

SEQ ID NO: 120 - RNA, 25 nt, synthetic construct
  source 1..25: mol_type = other RNA
tcccaaatagactccatgattcgta

SEQ ID NO: 121 - RNA, 25 nt, synthetic construct
  source 1..25: mol_type = other RNA
tttgtaattcccaagaatgtgcccg

SEQ ID NO: 122 - RNA, 25 nt, synthetic construct
  source 1..25: mol_type = other RNA
gtgaaataacttgggaactgacgtc

SEQ ID NO: 123 - RNA, 25 nt, synthetic construct
  source 1..25: mol_type = other RNA
taatttaaaccatagaacactaact

SEQ ID NO: 124 - RNA, 25 nt, synthetic construct
  source 1..25: mol_type = other RNA
tgaactaaatagcttaggatatgct

SEQ ID NO: 125 - RNA, 25 nt, synthetic construct
  source 1..25: mol_type = other RNA
ttgattttgatactgaactcaagcc

SEQ ID NO: 126 - RNA, 25 nt, synthetic construct
  source 1..25: mol_type = other RNA
acataatagagaagctgaactttgc

SEQ ID NO: 127 - RNA, 25 nt, synthetic construct
  source 1..25: mol_type = other RNA
ttaaattataacctgggaatggagc

SEQ ID NO: 128 - RNA, 25 nt, synthetic construct
  source 1..25: mol_type = other RNA
atattcataaccctggaacaagacc

SEQ ID NO: 129 - RNA, 22 nt, synthetic construct
  source 1..22: mol_type = other RNA
gtttcgttcataccttagttgc

SEQ ID NO: 130 - RNA, 25 nt, synthetic construct
  source 1..25: mol_type = other RNA
aaagtttcgttcataccttagttgc

SEQ ID NO: 131 - RNA, 22 nt, synthetic construct
  source 1..22: mol_type = other RNA
ctaagcatacgcgatattgagc

SEQ ID NO: 132 - RNA, 25 nt, synthetic construct
  source 1..25: mol_type = other RNA
aacctaagcatacgcgatattgagc

SEQ ID NO: 133 - RNA, 19 nt, synthetic construct
  source 1..19: mol_type = other RNA
ggctggagtaatctacaat

SEQ ID NO: 134 - RNA, 20 nt, synthetic construct
  source 1..20: mol_type = other RNA
gctggggttatctacaatca

SEQ ID NO: 135 - RNA, 19 nt, synthetic construct
  source 1..19: mol_type = other RNA
gttggccttgtgaatgtat

SEQ ID NO: 136 - RNA, 19 nt, synthetic construct
  source 1..19: mol_type = other RNA
ccatatgagatgccagcat

SEQ ID NO: 137 - RNA, 20 nt, synthetic construct
  source 1..20: mol_type = other RNA
ggatttatgatggaacatct

SEQ ID NO: 138 - RNA, 19 nt, synthetic construct
  source 1..19: mol_type = other RNA
cgctggtttatgaacggaa

SEQ ID NO: 139 - RNA, 19 nt, synthetic construct
  source 1..19: mol_type = other RNA
ccgttggagactggatcta

SEQ ID NO: 140 - RNA, 20 nt, synthetic construct
  source 1..20: mol_type = other RNA
ggggtaggttcaggagttta

SEQ ID NO: 141 - RNA, 20 nt, synthetic construct
  source 1..20: mol_type = other RNA
gatctatgacggaacatcta

SEQ ID NO: 142 - RNA, 22 nt, synthetic construct
  source 1..22: mol_type = other RNA
gccgggaattaatctacaatca

SEQ ID NO: 143 - RNA, 22 nt, synthetic construct
  source 1..22: mol_type = other RNA
gatctaatgaacggaacatcta

SEQ ID NO: 144 - RNA, 22 nt, synthetic construct
  source 1..22: mol_type = other RNA
ggagcatggtttcaggagttta

SEQ ID NO: 145 - RNA, 20 nt, synthetic construct
  source 1..20: mol_type = other RNA
agcggcctggttgactttcc

SEQ ID NO: 146 - RNA, 20 nt, synthetic construct
  source 1..20: mol_type = other RNA
agcgctttggagcatatatc

SEQ ID NO: 147 - RNA, 20 nt, synthetic construct
  source 1..20: mol_type = other RNA
ggaagtggagaattcaagtt

SEQ ID NO: 148 - RNA, 20 nt, synthetic construct
  source 1..20: mol_type = other RNA
agcgggagattcaagtttaa

SEQ ID NO: 149 - RNA, 20 nt, synthetic construct
  source 1..20: mol_type = other RNA
gcaggagagttaagtttaag

SEQ ID NO: 150 - RNA, 20 nt, synthetic construct
  source 1..20: mol_type = other RNA
gcttggagaattcctgctaa

SEQ ID NO: 151 - RNA, 20 nt, synthetic construct
  source 1..20: mol_type = other RNA
gcattgaggatcatcaacaa

SEQ ID NO: 152 - RNA, 20 nt, synthetic construct
  source 1..20: mol_type = other RNA
ggatttgctggctcaaaaat

SEQ ID NO: 153 - RNA, 20 nt, synthetic construct
  source 1..20: mol_type = other RNA
gcgggatatcagacacattt

SEQ ID NO: 154 - RNA, 20 nt, synthetic construct
  source 1..20: mol_type = other RNA
gaatgttgttacctaggata

SEQ ID NO: 155 - RNA, 23 nt, synthetic construct
  source 1..23: mol_type = other RNA
agcggcctggttgactttcccca

SEQ ID NO: 156 - RNA, 23 nt, synthetic construct
  source 1..23: mol_type = other RNA
agcgctttggagcatatatcaga

SEQ ID NO: 157 - RNA, 23 nt, synthetic construct
  source 1..23: mol_type = other RNA
ggaagtggagaattcaagtttaa

SEQ ID NO: 158 - RNA, 23 nt, synthetic construct
  source 1..23: mol_type = other RNA
agcgggagattcaagtttaagac

SEQ ID NO: 159 - RNA, 23 nt, synthetic construct
  source 1..23: mol_type = other RNA
gcaggagagttaagtttaagaca

SEQ ID NO: 160 - RNA, 23 nt, synthetic construct
  source 1..23: mol_type = other RNA
gcttggagaattcctgctaatgg

SEQ ID NO: 161 - RNA, 23 nt, synthetic construct
  source 1..23: mol_type = other RNA
gcattgaggatcatcaacaacac

SEQ ID NO: 162 - RNA, 23 nt, synthetic construct
  source 1..23: mol_type = other RNA
ggatttgctggctcaaaaatctc

SEQ ID NO: 163 - RNA, 23 nt, synthetic construct
  source 1..23: mol_type = other RNA
gcgggatatcagacacatttaca

SEQ ID NO: 164 - RNA, 23 nt, synthetic construct
  source 1..23: mol_type = other RNA
gaatgttgttacctaggatatgt

SEQ ID NO: 165 - RNA, 20 nt, synthetic construct
  source 1..20: mol_type = other RNA
ggagagatttcggtttaaac

SEQ ID NO: 166 - RNA, 20 nt, synthetic construct
  source 1..20: mol_type = other RNA
tacggatatgagtctatttg

SEQ ID NO: 167 - RNA, 20 nt, synthetic construct
  source 1..20: mol_type = other RNA
cgggtagttttgggaattac

SEQ ID NO: 168 - RNA, 20 nt, synthetic construct
  source 1..20: mol_type = other RNA
gacgttgttcgaagttattt

SEQ ID NO: 169 - RNA, 20 nt, synthetic construct
  source 1..20: mol_type = other RNA
agttgggtttgtggtttaaa

SEQ ID NO: 170 - RNA, 20 nt, synthetic construct
  source 1..20: mol_type = other RNA
agcatgtctagctatttagt

SEQ ID NO: 171 - RNA, 20 nt, synthetic construct
  source 1..20: mol_type = other RNA
ggtttggttggtatcaaaat

SEQ ID NO: 172 - RNA, 20 nt, synthetic construct
  source 1..20: mol_type = other RNA
gcagagtcgcttctctatta

SEQ ID NO: 173 - RNA, 20 nt, synthetic construct
  source 1..20: mol_type = other RNA
gctccgttcaggttataatt

SEQ ID NO: 174 - RNA, 20 nt, synthetic construct
  source 1..20: mol_type = other RNA
ggtcttttcggggttgtgaa

SEQ ID NO: 175 - RNA, 23 nt, synthetic construct
  source 1..23: mol_type = other RNA
ggagagatttcggtttaaacaga

SEQ ID NO: 176 - RNA, 23 nt, synthetic construct
  source 1..23: mol_type = other RNA
tacggatatgagtctatttggga

SEQ ID NO: 177 - RNA, 23 nt, synthetic construct
  source 1..23: mol_type = other RNA
cgggtagttttgggaattacaaa

SEQ ID NO: 178 - RNA, 23 nt, synthetic construct
  source 1..23: mol_type = other RNA
gacgttgttcgaagttatttcac

SEQ ID NO: 179 - RNA, 23 nt, synthetic construct
  source 1..23: mol_type = other RNA
agttgggtttgtggtttaaatta

SEQ ID NO: 180 - RNA, 23 nt, synthetic construct
  source 1..23: mol_type = other RNA
agcatgtctagctatttagttca

SEQ ID NO: 181 - RNA, 23 nt, synthetic construct
  source 1..23: mol_type = other RNA
ggtttggttggtatcaaaatcaa

SEQ ID NO: 182 - RNA, 23 nt, synthetic construct
  source 1..23: mol_type = other RNA
gcagagtcgcttctctattatgt

SEQ ID NO: 183 - RNA, 23 nt, synthetic construct
  source 1..23: mol_type = other RNA
gctccgttcaggttataatttaa

SEQ ID NO: 184 - RNA, 23 nt, synthetic construct
  source 1..23: mol_type = other RNA
ggtcttttcggggttgtgaatat

SEQ ID NO: 185 - RNA, 22 nt, synthetic construct
  source 1..22: mol_type = other RNA
gcaactaaggtatgaacgaaac

SEQ ID NO: 186 - RNA, 25 nt, synthetic construct
  source 1..25: mol_type = other RNA
gcaactaaggtatgaacgaaacttt

SEQ ID NO: 187 - RNA, 22 nt, synthetic construct
  source 1..22: mol_type = other RNA
gctcaatatcgcgtatgcttag

SEQ ID NO: 188 - RNA, 25 nt, synthetic construct
  source 1..25: mol_type = other RNA
gctcaatatcgcgtatgcttaggtt

SEQ ID NO: 189 - RNA, 116 nt, synthetic construct
  source 1..116: mol_type = other RNA
ctggaggcttgctttgggctgtatgctgattgtagattaattcccggccttttggcctctgactgaggctggagtaatctacaatcaggacacaaggccctttatcagcactcaca

SEQ ID NO: 190 - RNA, 118 nt, synthetic construct
  source 1..118: mol_type = other RNA
ctggaggcttgctttgggctgtatgctgtgattgtagattaattcccggcttttggcctctgactgagctggggttatctacaatcacaggacacaaggccctttatcagcactcaca

SEQ ID NO: 191 - RNA, 116 nt, synthetic construct
  source 1..116: mol_type = other RNA
ctggaggcttgctttgggctgtatgctgatacattcaccaggagccagcttttggcctctgactgagttggccttgtgaatgtatcaggacacaaggccctttatcagcactcaca

SEQ ID NO: 192 - RNA, 116 nt, synthetic construct
  source 1..116: mol_type = other RNA
ctggaggcttgctttgggctgtatgctgatgctggcatactccgtgtggttttggcctctgactgaccatatgagatgccagcatcaggacacaaggccctttatcagcactcaca

SEQ ID NO: 193 - RNA, 118 nt, synthetic construct
  source 1..118: mol_type = other RNA
ctggaggcttgctttgggctgtatgctgagatgttccgttcattagatccttttggcctctgactgaggatttatgatggaacatctcaggacacaaggccctttatcagcactcaca

SEQ ID NO: 194 - RNA, 116 nt, synthetic construct
  source 1..116: mol_type = other RNA
ctggaggcttgctttgggctgtatgctgttccgttcattagatccagtgttttggcctctgactgacgctggtttatgaacggaacaggacacaaggccctttatcagcactcaca

SEQ ID NO: 195 - RNA, 116 nt, synthetic construct
  source 1..116: mol_type = other RNA
ctggaggcttgctttgggctgtatgctgtagatccagtgttctcagcggttttggcctctgactgaccgttggagactggatctacaggacacaaggccctttatcagcactcaca

SEQ ID NO: 196 - RNA, 118 nt, synthetic construct
  source 1..118: mol_type = other RNA
ctggaggcttgctttgggctgtatgctgtaaactcctgaaaccatgctccttttggcctctgactgaggggtaggttcaggagtttacaggacacaaggccctttatcagcactcaca

SEQ ID NO: 197 - RNA, 118 nt, synthetic construct
  source 1..118: mol_type = other RNA
ctggaggcttgctttgggctgtatgctgtagatgttccgttcattagatcttttggcctctgactgagatctatgacggaacatctacaggacacaaggccctttatcagcactcaca

SEQ ID NO: 198 - RNA, 125 nt, synthetic construct
  source 1..125: mol_type = other RNA
gagaaggtatattgctgttgacagtgagcgtgattgtagattaattcccggctagtgaagccacagatgtagccgggaattaatctacaatcatgcctactgcctcggacttcaaggggctagaa

SEQ ID NO: 199 - RNA, 125 nt, synthetic construct
  source 1..125: mol_type = other RNA
gagaaggtatattgctgttgacagtgagcgtagatgttccgttcattagatctagtgaagccacagatgtagatctaatgaacggaacatctatgcctactgcctcggacttcaaggggctagaa

SEQ ID NO: 200 - RNA, 125 nt, synthetic construct
  source 1..125: mol_type = other RNA
gagaaggtatattgctgttgacagtgagcgtaaactcctgaaaccatgctcctagtgaagccacagatgtaggagcatggtttcaggagtttatgcctactgcctcggacttcaaggggctagaa

SEQ ID NO: 201 - RNA, 118 nt, synthetic construct
  source 1..118: mol_type = other RNA
ctggaggcttgctttgggctgtatgctgggaaagtcgacatagagccgctttttggcctctgactgaagcggcctggttgactttcccaggacacaaggccctttatcagcactcaca

SEQ ID NO: 202 - RNA, 118 nt, synthetic construct
  source 1..118: mol_type = other RNA
ctggaggcttgctttgggctgtatgctggatatatgctctcaagggtgctttttggcctctgactgaagcgctttggagcatatatccaggacacaaggccctttatcagcactcaca

SEQ ID NO: 203 - RNA, 118 nt, synthetic construct
  source 1..118: mol_type = other RNA
ctggaggcttgctttgggctgtatgctgaacttgagttcttcctgcttccttttggcctctgactgaggaagtggagaattcaagttcaggacacaaggccctttatcagcactcaca

SEQ ID NO: 204 - RNA, 118 nt, synthetic construct
  source 1..118: mol_type = other RNA
ctggaggcttgctttgggctgtatgctgttaaacttgagttcttcctgctttttggcctctgactgaagcgggagattcaagtttaacaggacacaaggccctttatcagcactcaca

SEQ ID NO: 205 - RNA, 118 nt, synthetic construct
  source 1..118: mol_type = other RNA
ctggaggcttgctttgggctgtatgctgcttaaacttgagttcttcctgcttttggcctctgactgagcaggagagttaagtttaagcaggacacaaggccctttatcagcactcaca

SEQ ID NO: 206 - RNA, 118 nt, synthetic construct
  source 1..118: mol_type = other RNA
ctggaggcttgctttgggctgtatgctgttagcaggagtttctatcaagcttttggcctctgactgagcttggagaattcctgctaacaggacacaaggccctttatcagcactcaca

SEQ ID NO: 207 - RNA, 118 nt, synthetic construct
  source 1..118: mol_type = other RNA
ctggaggcttgctttgggctgtatgctgttgttgatgatttctccagtgcttttggcctctgactgagcattgaggatcatcaacaacaggacacaaggccctttatcagcactcaca

SEQ ID NO: 208 - RNA, 118 nt, synthetic construct
  source 1..118: mol_type = other RNA
ctggaggcttgctttgggctgtatgctgatttttgagctgagcaagatccttttggcctctgactgaggatttgctggctcaaaaatcaggacacaaggccctttatcagcactcaca

SEQ ID NO: 209 - RNA, 118 nt, synthetic construct
  source 1..118: mol_type = other RNA
ctggaggcttgctttgggctgtatgctgaaatgtgtctgtatgttcctgcttttggcctctgactgagcgggatatcagacacatttcaggacacaaggccctttatcagcactcaca

SEQ ID NO: 210 - RNA, 118 nt, synthetic construct
  source 1..118: mol_type = other RNA
ctggaggcttgctttgggctgtatgctgtatcctaggtacatacacgttcttttggcctctgactgagaatgttgttacctaggatacaggacacaaggccctttatcagcactcaca

SEQ ID NO: 211 - RNA, 111 nt, synthetic construct
  source 1..111: mol_type = other RNA
ctggaggcttgctttgcgaatctctggggaaagtcgacatagagccgcttgtgttattcttgaagcggcctggttgactttccccagcgattcgctttatcgtcactcaca

SEQ ID NO: 212 - RNA, 111 nt, synthetic construct
  source 1..111: mol_type = other RNA
ctggaggcttgctttgcgaatctctctgatatatgctctcaagggtgcttgtgttattcttgaagcgctttggagcatatatcagagcgattcgctttatcgtcactcaca

SEQ ID NO: 213 - RNA, 111 nt, synthetic construct
  source 1..111: mol_type = other RNA
ctggaggcttgctttgcgaatctcttaaacttgagttcttcctgcttcctgtgttattcttgaggaagtggagaattcaagtttaagcgattcgctttatcgtcactcaca

SEQ ID NO: 214 - RNA, 111 nt, synthetic construct
  source 1..111: mol_type = other RNA
ctggaggcttgctttgcgaatctcgtcttaaacttgagttcttcctgcttgtgttattcttgaagcgggagattcaagtttaagacgcgattcgctttatcgtcactcaca

SEQ ID NO: 215 - RNA, 111 nt, synthetic construct
  source 1..111: mol_type = other RNA
ctggaggcttgctttgcgaatctctgtcttaaacttgagttcttcctgctgtgttattcttgagcaggagagttaagtttaagacagcgattcgctttatcgtcactcaca

SEQ ID NO: 216 - RNA, 111 nt, synthetic construct
  source 1..111: mol_type = other RNA
ctggaggcttgctttgcgaatctcccattagcaggagtttctatcaagctgtgttattcttgagcttggagaattcctgctaatgggcgattcgctttatcgtcactcaca

SEQ ID NO: 217 - RNA, 111 nt, synthetic construct
  source 1..111: mol_type = other RNA
ctggaggcttgctttgcgaatctcgtgttgttgatgatttctccagtgctgtgttattcttgagcattgaggatcatcaacaacacgcgattcgctttatcgtcactcaca

SEQ ID NO: 218 - RNA, 111 nt, synthetic construct
  source 1..111: mol_type = other RNA
ctggaggcttgctttgcgaatctcgagatttttgagctgagcaagatcctgtgttattcttgaggatttgctggctcaaaaatctcgcgattcgctttatcgtcactcaca

SEQ ID NO: 219 - RNA, 111 nt, synthetic construct
  source 1..111: mol_type = other RNA
ctggaggcttgctttgcgaatctctgtaaatgtgtctgtatgttcctgctgtgttattcttgagcgggatatcagacacatttacagcgattcgctttatcgtcactcaca

SEQ ID NO: 220 - RNA, 111 nt, synthetic construct
  source 1..111: mol_type = other RNA
ctggaggcttgctttgcgaatctcacatatcctaggtacatacacgttctgtgttattcttgagaatgttgttacctaggatatgtgcgattcgctttatcgtcactcaca

SEQ ID NO: 221 - RNA, 118 nt, synthetic construct
  source 1..118: mol_type = other RNA
ctggaggcttgctttgggctgtatgctggtttaaaccgagaatattctccttttggcctctgactgaggagagatttcggtttaaaccaggacacaaggccctttatcagcactcaca

SEQ ID NO: 222 - RNA, 118 nt, synthetic construct
  source 1..118: mol_type = other RNA
ctggaggcttgctttgggctgtatgctgcaaatagactccatgattcgtattttggcctctgactgatacggatatgagtctatttgcaggacacaaggccctttatcagcactcaca

SEQ ID NO: 223 - RNA, 118 nt, synthetic construct
  source 1..118: mol_type = other RNA
ctggaggcttgctttgggctgtatgctggtaattcccaagaatgtgcccgttttggcctctgactgacgggtagttttgggaattaccaggacacaaggccctttatcagcactcaca

SEQ ID NO: 224 - RNA, 118 nt, synthetic construct
  source 1..118: mol_type = other RNA
ctggaggcttgctttgggctgtatgctgaaataacttgggaactgacgtcttttggcctctgactgagacgttgttcgaagttatttcaggacacaaggccctttatcagcactcaca

SEQ ID NO: 225 - RNA, 118 nt, synthetic construct
  source 1..118: mol_type = other RNA
ctggaggcttgctttgggctgtatgctgtttaaaccatagaacactaactttttggcctctgactgaagttgggtttgtggtttaaacaggacacaaggccctttatcagcactcaca

SEQ ID NO: 226 - RNA, 118 nt, synthetic construct
  source 1..118: mol_type = other RNA
ctggaggcttgctttgggctgtatgctgactaaatagcttaggatatgctttttggcctctgactgaagcatgtctagctatttagtcaggacacaaggccctttatcagcactcaca

SEQ ID NO: 227 - RNA, 118 nt, synthetic construct
  source 1..118: mol_type = other RNA
ctggaggcttgctttgggctgtatgctgattttgatactgaactcaagccttttggcctctgactgaggtttggttggtatcaaaatcaggacacaaggccctttatcagcactcaca

SEQ ID NO: 228 - RNA, 118 nt, synthetic construct
  source 1..118: mol_type = other RNA
ctggaggcttgctttgggctgtatgctgtaatagagaagctgaactttgcttttggcctctgactgagcagagtcgcttctctattacaggacacaaggccctttatcagcactcaca

SEQ ID NO: 229 - RNA, 118 nt, synthetic construct
  source 1..118: mol_type = other RNA
ctggaggcttgctttgggctgtatgctgaattataacctgggaatggagcttttggcctctgactgagctccgttcaggttataattcaggacacaaggccctttatcagcactcaca

SEQ ID NO: 230 - RNA, 118 nt, synthetic construct
  source 1..118: mol_type = other RNA
ctggaggcttgctttgggctgtatgctgttcataaccctggaacaagaccttttggcctctgactgaggtcttttcggggttgtgaacaggacacaaggccctttatcagcactcaca

SEQ ID NO: 231 - RNA, 111 nt, synthetic construct
  source 1..111: mol_type = other RNA
ctggaggcttgctttgcgaatctctctgtttaaaccgagaatattctcctgtgttattcttgaggagagatttcggtttaaacagagcgattcgctttatcgtcactcaca

SEQ ID NO: 232 - RNA, 111 nt, synthetic construct
  source 1..111: mol_type = other RNA
ctggaggcttgctttgcgaatctctcccaaatagactccatgattcgtatgtgttattcttgatacggatatgagtctatttgggagcgattcgctttatcgtcactcaca

SEQ ID NO: 233 - RNA, 111 nt, synthetic construct
  source 1..111: mol_type = other RNA
ctggaggcttgctttgcgaatctctttgtaattcccaagaatgtgcccgtgtgttattcttgacgggtagttttgggaattacaaagcgattcgctttatcgtcactcaca

SEQ ID NO: 234 - RNA, 111 nt, synthetic construct
  source 1..111: mol_type = other RNA
ctggaggcttgctttgcgaatctcgtgaaataacttgggaactgacgtctgtgttattcttgagacgttgttcgaagttatttcacgcgattcgctttatcgtcactcaca

SEQ ID NO: 235 - RNA, 111 nt, synthetic construct
  source 1..111: mol_type = other RNA
ctggaggcttgctttgcgaatctctaatttaaaccatagaacactaacttgtgttattcttgaagttgggtttgtggtttaaattagcgattcgctttatcgtcactcaca

SEQ ID NO: 236 - RNA, 111 nt, synthetic construct
  source 1..111: mol_type = other RNA
ctggaggcttgctttgcgaatctctgaactaaatagcttaggatatgcttgtgttattcttgaagcatgtctagctatttagttcagcgattcgctttatcgtcactcaca

SEQ ID NO: 237 - RNA, 111 nt, synthetic construct
  source 1..111: mol_type = other RNA
ctggaggcttgctttgcgaatctcttgattttgatactgaactcaagcctgtgttattcttgaggtttggttggtatcaaaatcaagcgattcgctttatcgtcactcaca

SEQ ID NO: 238 - RNA, 111 nt, synthetic construct
  source 1..111: mol_type = other RNA
ctggaggcttgctttgcgaatctcacataatagagaagctgaactttgctgtgttattcttgagcagagtcgcttctctattatgtgcgattcgctttatcgtcactcaca

SEQ ID NO: 239 - RNA, 111 nt, synthetic construct
  source 1..111: mol_type = other RNA
ctggaggcttgctttgcgaatctcttaaattataacctgggaatggagctgtgttattcttgagctccgttcaggttataatttaagcgattcgctttatcgtcactcaca

SEQ ID NO: 240 - RNA, 111 nt, synthetic construct
  source 1..111: mol_type = other RNA
ctggaggcttgctttgcgaatctcatattcataaccctggaacaagacctgtgttattcttgaggtcttttcggggttgtgaatatgcgattcgctttatcgtcactcaca

SEQ ID NO: 241 - RNA, 120 nt, synthetic construct
  source 1..120: mol_type = other RNA
ctggaggcttgctttgggctgtatgctggtttcgttcataccttagttgcttttggcctctgactgagcaactaaggtatgaacgaaaccaggacacaaggccctttatcagcactcaca

SEQ ID NO: 242 - RNA, 113 nt, synthetic construct
  source 1..113: mol_type = other RNA
ctggaggcttgctttgcgaatctcaaagtttcgttcataccttagttgctgtgttattcttgagcaactaaggtatgaacgaaactttgcgattcgctttatcgtcactcaca

SEQ ID NO: 243 - RNA, 120 nt, synthetic construct
  source 1..120: mol_type = other RNA
ctggaggcttgctttgggctgtatgctgctaagcatacgcgatattgagcttttggcctctgactgagctcaatatcgcgtatgcttagcaggacacaaggccctttatcagcactcaca

SEQ ID NO: 244 - RNA, 113 nt, synthetic construct
  source 1..113: mol_type = other RNA
ctggaggcttgctttgcgaatctcaacctaagcatacgcgatattgagctgtgttattcttgagctcaatatcgcgtatgcttaggttgcgattcgctttatcgtcactcaca

SEQ ID NO: 245 - RNA, 28 nt, synthetic construct
  source 1..28: mol_type = other RNA
ctggaggcttgctttgggctgtatgctg

SEQ ID NO: 246 - RNA, 30 nt, synthetic construct
  source 1..30: mol_type = other RNA
gagaaggtatattgctgttgacagtgagcg

SEQ ID NO: 247 - RNA, 24 nt, synthetic construct
  source 1..24: mol_type = other RNA
ctggaggcttgctttgcgaatctc

SEQ ID NO: 248 - RNA, 17 nt, synthetic construct
  source 1..17: mol_type = other RNA
ttttggcctctgactga

SEQ ID NO: 249 - RNA, 19 nt, synthetic construct
  source 1..19: mol_type = other RNA
tagtgaagccacagatgta

SEQ ID NO: 250 - RNA, 14 nt, synthetic construct
  source 1..14: mol_type = other RNA
tgtgttattcttga

SEQ ID NO: 251 - RNA, 31 nt, synthetic construct
  source 1..31: mol_type = other RNA
caggacacaaggccctttatcagcactcaca

SEQ ID NO: 252 - RNA, 32 nt, synthetic construct
  source 1..32: mol_type = other RNA
tgcctactgcctcggacttcaaggggctagaa

SEQ ID NO: 253 - RNA, 25 nt, synthetic construct
  source 1..25: mol_type = other RNA
gcgattcgctttatcgtcactcaca

SEQ ID NO: 254 - RNA, 397 nt, synthetic construct
  source 1..397: mol_type = other RNA
gagaaggtatattgctgttgacagtgagcgatagatgttccgttcattagatctagtgaagccacagatgtagatctaatgaacggaacatctagtgcctactgcctcggacttcaaggggctaatgtctagaaaacgaaggtatattgctgttgacagtgagcgatgattgtagattaattcccggctagtgaagccacagatgtagccgggaattaatctacaatcagtgcctactgcctcggacttcaaggggctattgcggccgcaacgaaggtatattgctgttgacagtgagcgataaactcctgaaaccatgctcctagtgaagccacagatgtaggagcatggtttcaggagtttagtgcctactgcctcggacttcaaggggctagaa

SEQ ID NO: 255 - RNA, 393 nt, synthetic construct
  source 1..393: mol_type = other RNA
gagaaggtatattgctgttgacagtgagcgatagatgttccgttcattagatctagtgaagccacagatgtagatctaatgaacggaacatctagtgcctactgcctcggacttcaaggggctaatgtctagaaaacctggaggcttgctttgggctgtatgctgtgattgtagattaattcccggcttttggcctctgactgagctggggttatctacaatcacaggacacaaggccctttatcagcactcacattgcggccgcaacgaaggtatattgctgttgacagtgagcgataaactcctgaaaccatgctcctagtgaagccacagatgtaggagcatggtttcaggagtttagtgcctactgcctcggacttcaaggggctagaa

SEQ ID NO: 256 - RNA, 384 nt, synthetic construct
  source 1..384: mol_type = other RNA
ctggaggcttgctttgggctgtatgctgtagatgttccgttcattagatcttttggcctctgactgagatctatgacggaacatctacaggacacaaggccctttatcagcactcacaatgtctagaaaacgaaggtatattgctgttgacagtgagcgatgattgtagattaattcccggctagtgaagccacagatgtagccgggaattaatctacaatcagtgcctactgcctcggacttcaaggggctattgcggccgcaacctggaggcttgctttgggctgtatgctgtaaactcctgaaaccatgctccttttggcctctgactgaggggtaggttcaggagtttacaggacacaaggccctttatcagcactcaca

SEQ ID NO: 257 - RNA, 380 nt, synthetic construct
  source 1..380: mol_type = other RNA
ctggaggcttgctttgggctgtatgctgtagatgttccgttcattagatcttttggcctctgactgagatctatgacggaacatctacaggacacaaggccctttatcagcactcacaatgtctagaaaacctggaggcttgctttgggctgtatgctgtgattgtagattaattcccggcttttggcctctgactgagctggggttatctacaatcacaggacacaaggccctttatcagcactcacattgcggccgcaacctggaggcttgctttgggctgtatgctgtaaactcctgaaaccatgctccttttggcctctgactgaggggtaggttcaggagtttacaggacacaaggccctttatcagcactcaca

SEQ ID NO: 258 - RNA, 398 nt, synthetic construct
  source 1..398: mol_type = other RNA
ctggaggcttgctttgggctgtatgctgtagatgttccgttcattagatcttttggcctctgactgagatctatgacggaacatctacaggacacaaggccctttatcagcactcacaatgtctagaaaacctggaggcttgctttgggctgtatgctgtgattgtagattaattcccggcttttggcctctgactgagctggggttatctacaatcacaggacacaaggccctttatcagcactcacattgcggccgcaactctaccgggccaccgcacaccatgttgccagtctctaggtccctgagaccctttaacctgtgaggacatccagggtcacaggtgaggttcttgggagcctggcgtctggcccaaccacacacctggggaattgctg

SEQ ID NO: 259 - RNA, 401 nt, synthetic construct
  source 1..401: mol_type = other RNA
ctggaggcttgctttgggctgtatgctgtagatgttccgttcattagatcttttggcctctgactgagatctatgacggaacatctacaggacacaaggccctttatcagcactcacaatgtctagaaaacctggaggcttgctttgggctgtatgctgtgattgtagattaattcccggcttttggcctctgactgagctggggttatctacaatcacaggacacaaggccctttatcagcactcacattgcggccgcaactgaggaggcagggccggcatgcctctgctgctggccagagctcttttcacattgtgctactgtctgcacctgtcactagcagtgcaatgttaaaagggcattggccgtgtagtgctacccagcgctggctgcctcctca

SEQ ID NO: 260 - RNA, 377 nt, synthetic construct
  source 1..377: mol_type = other RNA
ctggaggcttgctttgggctgtatgctgtagatgttccgttcattagatcttttggcctctgactgagatctatgacggaacatctacaggacacaaggccctttatcagcactcacaatgtctagaaaacctggaggcttgctttgggctgtatgctgtgattgtagattaattcccggcttttggcctctgactgagctggggttatctacaatcacaggacacaaggccctttatcagcactcacattgcggccgcaacctgaaggcttgctgtaggctgtatgctgttaatgctaatcgtgataggggtttttgcctccaactgactcctacatattagcattaacagtgtatgatgcctgttactagcattc

SEQ ID NO: 261 - RNA, 389 nt, synthetic construct
  source 1..389: mol_type = other RNA
catcctgctgcctggcctccctgggctctgcctcccgtgcctactgagctgaaacacagttggtttgtgtacactggctcagttcagcaggaacaggggtcaagcccccttggagcctgcagcatgtctagaaaacctggaggcttgctttgggctgtatgctggtttcgttcataccttagttgcttttggcctctgactgagcaactaaggtatgaacgaaaccaggacacaaggccctttatcagcactcacattgcggccgcaacctggaggcttgctttgggctgtatgctgctaagcatacgcgatattgagcttttggcctctgactgagctcaatatcgcgtatgcttagcaggacacaaggccctttatcagcactcaca

SEQ ID NO: 262 - RNA, 403 nt, synthetic construct
  source 1..403: mol_type = other RNA
ttggcgctgaaggccactcgctcccaccttgtcctcacggtccagttttcccaggaatcccttagatgctaagatggggattcctggaaatactgttcttgaggtcatggtttcacagctggatttgcctccttcccatgtctagaaaacctggaggcttgctttgggctgtatgctggtttcgttcataccttagttgcttttggcctctgactgagcaactaaggtatgaacgaaaccaggacacaaggccctttatcagcactcacattgcggccgcaacctggaggcttgctttgggctgtatgctgctaagcatacgcgatattgagcttttggcctctgactgagctcaatatcgcgtatgcttagcaggacacaaggccctttatcagcactcaca

SEQ ID NO: 263 - RNA, 389 nt, synthetic construct
  source 1..389: mol_type = other RNA
gtccaggctgaaagaactttggccacctggcactgagaactgaattccataggctgtgagctctagcaatgccctgtggactcagttctggtgcccggcagtgctacaacatcaatgccaaggatgtctagaaaacctggaggcttgctttgggctgtatgctggtttcgttcataccttagttgcttttggcctctgactgagcaactaaggtatgaacgaaaccaggacacaaggccctttatcagcactcacattgcggccgcaacctggaggcttgctttgggctgtatgctgctaagcatacgcgatattgagcttttggcctctgactgagctcaatatcgcgtatgcttagcaggacacaaggccctttatcagcactcaca

SEQ ID NO: 264 - RNA, 396 nt, synthetic construct
  source 1..396: mol_type = other RNA
tgtcctcccgtcttggacctcagtcctgggggctccaaagtgctgttcgtgcaggtagtgtgattacccaacctactgctgagctagcacttcccgagcccccgggacacgttctctctgccaattgtctatgtctagaaaacctggaggcttgctttgggctgtatgctggtttcgttcataccttagttgcttttggcctctgactgagcaactaaggtatgaacgaaaccaggacacaaggccctttatcagcactcacattgcggccgcaacctggaggcttgctttgggctgtatgctgctaagcatacgcgatattgagcttttggcctctgactgagctcaatatcgcgtatgcttagcaggacacaaggccctttatcagcactcaca

SEQ ID NO: 265 - RNA, 416 nt, synthetic construct
  source 1..416: mol_type = other RNA
ttccccacagaagctcttggcctggcctcctgcagtgccacgctccgtgtatttgacaagctgagttggacactccatgtggtagagtgtcagtttgtcaaataccccaagtgcggcacatgcttaccagctctaggccagggcagatggatgtctagaaaacctggaggcttgctttgggctgtatgctggtttcgttcataccttagttgcttttggcctctgactgagcaactaaggtatgaacgaaaccaggacacaaggccctttatcagcactcacattgcggccgcaacctggaggcttgctttgggctgtatgctgctaagcatacgcgatattgagcttttggcctctgactgagctcaatatcgcgtatgcttagcaggacacaaggccctttatcagcactcaca

SEQ ID NO: 266 - RNA, 402 nt, synthetic construct
  source 1..402: mol_type = other RNA
catcctgctgcctggcctccctgggctctgcctcccgtgcctactgagctgaaacacagttggtttgtgtacactggctcagttcagcaggaacaggggtcaagcccccttggagcctgcagcatgtctagaaaactgtcctcccgtcttggacctcagtcctgggggctccaaagtgctgttcgtgcaggtagtgtgattacccaacctactgctgagctagcacttcccgagcccccgggacacgttctctctgccaattgtctttgcggccgcaacgtccaggctgaaagaactttggccacctggcactgagaactgaattccataggctgtgagctctagcaatgccctgtggactcagttctggtgcccggcagtgctacaacatcaatgccaagg

SEQ ID NO: 267 - RNA, 84 nt, synthetic construct
  source 1..84: mol_type = other RNA
gcatccgggttgaggtagtaggttgtatggtttagagttacaccctgggagttaactgtacaaccttctagctttccttggagc

SEQ ID NO: 268 - RNA, 85 nt, synthetic construct
  source 1..85: mol_type = other RNA
aggcctctctctccgtgttcacagcggaccttgatttaaatgtccatacaattaaggcacgcggtgaatgccaagaatggggctg

SEQ ID NO: 269 - RNA, 86 nt, synthetic construct
  source 1..86: mol_type = other RNA
tgccagtctctaggtccctgagaccctttaacctgtgaggacatccagggtcacaggtgaggttcttgggagcctggcgtctggcc

SEQ ID NO: 270 - RNA, 88 nt, synthetic construct
  source 1..88: mol_type = other RNA
tgcgctcctctcagtccctgagaccctaacttgtgatgtttaccgtttaaatccacgggttaggctcttgggagctgcgagtcgtgct

SEQ ID NO: 271 - RNA, 89 nt, synthetic construct
  source 1..89: mol_type = other RNA
tgctgctggccagagctcttttcacattgtgctactgtctgcacctgtcactagcagtgcaatgttaaaagggcattggccgtgtagtg

SEQ ID NO: 272 - RNA, 82 nt, synthetic construct
  source 1..82: mol_type = other RNA
ggcctgcccgacactctttccctgttgcactactataggccgctgggaagcagtgcaatgatgaaagggcatcggtcaggtc

SEQ ID NO: 273 - RNA, 87 nt, synthetic construct
  source 1..87: mol_type = other RNA
gacagtgcagtcacccataaagtagaaagcactactaacagcactggagggtgtagtgtttcctactttatggatgagtgtactgtg

SEQ ID NO: 274 - RNA, 88 nt, synthetic construct
  source 1..88: mol_type = other RNA
caccttgtcctcacggtccagttttcccaggaatcccttagatgctaagatggggattcctggaaatactgttcttgaggtcatggtt

SEQ ID NO: 275 - RNA, 99 nt, synthetic construct
  source 1..99: mol_type = other RNA
ccgatgtgtatcctcagctttgagaactgaattccatgggttgtgtcagtgtcagacctctgaaattcagttcttcagctgggatatctctgtcatcgt

SEQ ID NO: 276 - RNA, 73 nt, synthetic construct
  source 1..73: mol_type = other RNA
cctggcactgagaactgaattccataggctgtgagctctagcaatgccctgtggactcagttctggtgcccgg

SEQ ID NO: 277 - RNA, 65 nt, synthetic construct
  source 1..65: mol_type = other RNA
ctgttaatgctaatcgtgataggggtttttgcctccaactgactcctacatattagcattaacag

SEQ ID NO: 278 - RNA, 110 nt, synthetic construct
  source 1..110: mol_type = other RNA
tgagttttgaggttgcttcagtgaacattcaacgctgtcggtgagtttggaattaaaatcaaaaccatcgaccgttgattgtaccctatggctaaccatcatctactcca

SEQ ID NO: 279 - RNA, 88 nt, synthetic construct
  source 1..88: mol_type = other RNA
cgaggatgggagctgagggctgggtctttgcgggcgagatgagggtgtcggatcaactggcctacaaagtcccagttctcggcccccg

SEQ ID NO: 280 - RNA, 82 nt, synthetic construct
  source 1..82: mol_type = other RNA
gcagtcctctgttagttttgcatagttgcactacaagaagaatgtagttgtgcaaatctatgcaaaactgatggtggcctgc

SEQ ID NO: 281 - RNA, 72 nt, synthetic construct
  source 1..72: mol_type = other RNA
tgtcgggtagcttatcagactgatgttgactgttgaatctcatggcaacaccagtcgatgggctgtctgaca

SEQ ID NO: 282 - RNA, 110 nt, synthetic construct
  source 1..110: mol_type = other RNA
acccggcagtgcctccaggcgcagggcagcccctgcccaccgcacactgcgctgccccagacccactgtgcgtgtgacagcggctgatctgtgcctgggcagcgcgaccc

SEQ ID NO: 283 - RNA, 110 nt, synthetic construct
  source 1..110: mol_type = other RNA
cctggcctcctgcagtgccacgctccgtgtatttgacaagctgagttggacactccatgtggtagagtgtcagtttgtcaaataccccaagtgcggcacatgcttaccag

SEQ ID NO: 284 - RNA, 73 nt, synthetic construct
  source 1..73: mol_type = other RNA
ggccggctggggttcctggggatgggatttgcttcctgtcacaaatcacattgccagggatttccaaccgacc

SEQ ID NO: 285 - RNA, 73 nt, synthetic construct
  source 1..73: mol_type = other RNA
ctctgcctcccgtgcctactgagctgaaacacagttggtttgtgtacactggctcagttcagcaggaacaggg

SEQ ID NO: 286 - RNA, 77 nt, synthetic construct
  source 1..77: mol_type = other RNA
gtggcctcgttcaagtaatccaggataggctgtgcaggtcccaatgggcctattcttggttacttgcacggggacgc

SEQ ID NO: 287 - RNA, 77 nt, synthetic construct
  source 1..77: mol_type = other RNA
ccgggacccagttcaagtaattcaggataggttgtgtgctgtccagcctgttctccattacttggctcggggaccgg

SEQ ID NO: 288 - RNA, 78 nt, synthetic construct
  source 1..78: mol_type = other RNA
ctgaggagcagggcttagctgcttgtgagcagggtccacaccaagtcgtgttcacagtggctaagttccgccccccag

SEQ ID NO: 289 - RNA, 97 nt, synthetic construct
  source 1..97: mol_type = other RNA
acctctctaacaaggtgcagagcttagctgattggtgaacagtgattggtttccgctttgttcacagtggctaagttctgcacctgaagagaaggtg

SEQ ID NO: 290 - RNA, 71 nt, synthetic construct
  source 1..71: mol_type = other RNA
gcgactgtaaacatcctcgactggaagctgtgaagccacagatgggctttcagtcggatgtttgcagctgc

SEQ ID NO: 291 - RNA, 88 nt, synthetic construct
  source 1..88: mol_type = other RNA
accaagtttcagttcatgtaaacatcctacactcagctgtaatacatggattggctgggaggtggatgtttacttcagctgacttgga

SEQ ID NO: 292 - RNA, 66 nt, synthetic construct
  source 1..66: mol_type = other RNA
agggctcctgactccaggtcctgtgtgttacctagaaatagcactggacttggagtcagaaggcct

SEQ ID NO: 293 - RNA, 87 nt, synthetic construct
  source 1..87: mol_type = other RNA
caatagacacccatcgtgtcttttgctctgcagtcagtaaatatttttttgtgaatgtgtagcaaaagacagaatggtggtccattg

SEQ ID NO: 294 - RNA, 89 nt, synthetic construct
  source 1..89: mol_type = other RNA
cggggttggttgttatctttggttatctagctgtatgagtggtgtggagtcttcataaagctagataaccgaaagtaaaaataacccca

SEQ ID NO: 295 - RNA, 80 nt, synthetic construct
  source 1..80: mol_type = other RNA
ctgggggctccaaagtgctgttcgtgcaggtagtgtgattacccaacctactgctgagctagcacttcccgagcccccgg

SEQ ID NO: 296 - RNA, 4201 nt, Homo sapiens
  source 1..4201: mol_type = genomic RNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 297 - RNA, 3963 nt, Homo sapiens
  source 1..3963: mol_type = genomic RNA
ggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacaccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctgaaaa

SEQ ID NO: 298 - RNA, 7218 nt, Homo sapiens
  source 1..7218: mol_type = genomic RNA
gcacttccttctcggctagattatctgaaactgttgtcggttcttgagatgatactaccaccgaatgtctgtgtttcattgtctagtccaacctgtattgtggatatctacaacgttccggcaatagttttgcaggtgcatcacatttttgtttttgttttgggaggaaaagggagggcacggcagccaggcttcatattcctacaagtgcatgcttcaagattactgtacttacagtgtttccaacatcttctcataaaaggggaaagcttcatagcctcaaccatgaaggaaaccagtcgcatagggcatggagctggagaactataaacagcccgtggtgctgagagaggacaactgccgaaggcgccggaggatgaagccgcgcagtgctgcggccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccactggtggatgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcagcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctaatactttaggctagaatcaaaaacaagttagtgttctatggtttaaattagcatagcaatcatcgaacttggatttcaaatgcaatagacattgtgaaagctggcatttcagaagtatagctcttttcctacctgaactcttccctggagaaaagatgttggcattgctgattgtttggttaagcaatgtccagtgctaggattatttgcaggtttggttttttctcatttgtctgtggcattggagaatattctcggtttaaacagactaatgacttccttattgtccctgatattttgactatcttactattgagtgcttctggaaattctttggaataattgatgacatctattttcatctgggtttagtctcaattttggttatctttgtgttcctcaagctctttaaagaaaaagatgtaatcgttgtaacctttgtctcattccttaaatgatgcttccaaacatctccttagtgtctgcaggtgttagtggtgtgctaaaagcaaggaaagcgagttagtcttttcagtgtcttttgcaattcaattcttttgtcatgtataactgagacacacaaacacagcaggagaaatctaaaccgttgtgccttgaccttcctctgctggtcttgttccagggttatgaatatgaaaaaatagagatgagactttttgtgtcaactctgtccacaagagtgagttatctagtatgattagtatagctttctccagcatggcagcaggaagtaactacagggcctcttttatgcctgacatttcttcccttcctttttccctgcctccctttttcatcaattgcgatgctcccacaactctttacagacttgtgaaatcttcaagaacacctttactctataactcaaaaattagttgaaaaataattacttctcaaggattattagaatcttaggtacttatttgtaaagatgtttagtgacttttttttcaagtatcttattaaaggaggcattctagaaaatatgaattagtttccaaatgccttaattttaaactttggcctgaacagttttttctttttcttaatggaagaagatatttaatatcttaaaaatattccaagttaggaagaacactacttgccttatccatttcccatttaaaggacttttaaactttgacacatccttcagatttcctgaaaataattgaaatatcttactttaaaaatattttcatctctgaaatatctcgttatttattggaggtattgtttaaccttagagagaccattaaattatttataaaatattttgtaattacctgtagctaatacattacatagaaaaaaactatgttaacagtgtctctgtttaagtataatcagatataaatatatacttaattttttaattttaaaaatagatacctgtttgactttgaggtagtccagaccttttctttttttttttttttttaatgtgtgcaaaagcccaaaggttcctaagcctggctgcaaagaagaatcaacagggacactttttaaaaacactcttatcagcctgggcaacacagtgagactccatctcttaaaaaaaaaattagctgggtatagtggtatgtgcctgtagtcccaggtactcaggaggctgaggcaggaggattgcctgagcccaggaggtggaaactgcagagagtcatgatcatgtccttacactccagcctggataacagagcgagaccctgtctcaaaaaaataaaataaaaaataaaaacacccttgcctgcgctccattcccaggttataatttaattactgtgggatgagacccagacatcaatatttttctagattcttcaggtgattctaattcacagccagagttggaagccctgcgtggcctttgaaggtctagatgattcttcttccttgccctttgagcttttccccatctcacaggtatctagaaaaaaactcctcttctttggcaacctgtccttttaaaatcacactctacccacctgtacagaagaccatgccctataatgaaatgtttattcctatctataaatggaggataaacattttgtggcacttctggaccaactattccctactattcttttgaagaaaggcaggaagagtactttctaattcagaagaggatgttttcactattctgataaacaatagccaagttcagaccttgtacagattctttttatttgaattgctgaaataatttattgatgatgaaaaaaagattagagaggaatacatttatatttagcttattggcacatgtgcatacatatttcctcttcaaatgaaccagttctttcatttcattatgctaatatatatatataacatatatatgctaatatatatataacatatatatgctaatatatatatatcacacatatatatcacagttttatatatatatatgtatgtgtgtgtgtatatatatatatatatatcacagttgggcttgattcttccgtattccaaagagcataatttcagttctatagacttatagataaataaaaatcatctttgtgggcttccttcctttactgttcgcagtgaattacatgacgaacaacttctatacctttgaaaatgttctagaactagaatcatcctgctactgggaactacccacagctctatcttcaatgccaggtgaaaacacagatcacaagtcagatgaatcaggccaaagcaacttttatgtatatctaggactggcgacataatttgcagagcccagtgaacagtgaaaatgcagtcccatcattcaaaaattattaagaaattcgagatggcaacagcagttgtcacaccaagcacagtgcccttctgagcatgaggctctgtgtgactgcatgggtccatgagaccagcactgtgtgggtctgaatgatggctttggtgttcagtttagcacacgcggtctaccacgtctgcatgagtggtaaatgtagtgcctgggtcaaaatgttccttctgttcaaatcgaaatacctcatctctatggctctatggctgtacattaggacctagaacagtggcccattgctcttagactggaaccatgtccactaaaataaacctaagcagatgttgtagacctagccccacaggactgcatttagctgcttcagtgacactttgatgaaagtatggagaagtggagacattatagataaaatatatcaattcccagagaaaactcttgacttaaaaacttaactgtagtaaatatatctttttcaggtgatgaattatttttttaaaaaaggttacatataggaattctgcagtataatttggaggctattagtgctatattaatggaaattaattattttttaagtaagtccaaaaaataatctagaaagtaagtttccagagcaaatctgacctagcatttggtatgctaggctctgcttttcatgattttgaaataaatcataattagacttaacaatatggagaaaataaacttgtatttttaagtgttctgttggcttattttctgtttcatccaactcaataattctgataaataaatttggttctagtttggtgcttgaaaaaaaa

SEQ ID NO: 299 - RNA, 2586 nt, Homo sapiens
  source 1..2586: mol_type = genomic RNA
acagtagcccggcggcccagggcaatccgaccacatttcactctcaccgctgtaggaatccagatgcaggccaagtacagcagcacgagggacatgctggatgatgatggggacaccaccatgagcctgcattctcaaggctctgccacaactcggcatccagagccccggcgcacagtttttcagtactaccagctctccaatactggtcaagacaccatttctcaaatggaagaaagattaggaaatacgtcccaagagttgcaatctcttcaagtccagaatataaagcttgcaggaagtctgcagcatgtggctgaaaaactctgtcgtgagctgtataacaaagctggagcacacaggtgcagcccttgtacagaacaatggaaatggcatggagacaattgctaccagttctataaagacagcaaaagttgggaggactgtaaatatttctgccttagtgaaaactctaccatgctgaagataaacaaacaagaagacctggaatttgccgcgtctcagagctactctgagtttttctactcttattggacagggcttttgcgccctgacagtggcaaggcctggctgtggatggatggaacccctttcacttctgaactgttccatattataatagatgtcaccagcccaagaagcagagactgtgtggccatccttaatgggatgatcttctcaaaggactgcaaagaattgaagcgttgtgtctgtgagagaagggcaggaatggtgaagccagagagcctccatgtcccccctgaaacattaggcgaaggtgactgattcgccctctgcaactacaaatagcagagtgagccaggcggtgccaaagcaagggctagttgagacattgggaaatggaacataatcaggaaagactatctctctgactagtacaaaatgggttctcgtgtttcctgttcaggatcaccagcatttctgagcttgggtttatgcacgtatttaacagtcacaagaagtcttatttacatgccaccaaccaacctcagaaacccataatgtcatctgccttcttggcttagagataacttttagctctctttcttctcaatgtctaatatcacctccctgttttcatgtcttccttacacttggtggaataagaaactttttgaagtagaggaaatacattgaggtaacatccttttctctgacagtcaagtagtccatcagaaattggcagtcacttcccagattgtaccagcaaatacacaaggaattctttttgtttgtttcagttcatactagtcccttcccaatccatcagtaaagaccccatctgccttgtccatgccgtttcccaacagggatgtcacttgatatgagaatctcaaatctcaatgccttataagcattccttcctgtgtccattaagactctgataattgtctcccctccataggaatttctcccaggaaagaaatatatccccatctccgtttcatatcagaactaccgtccccgatattcccttcagagagattaaagaccagaaaaaagtgagcctcttcatctgcacctgtaatagtttcagttcctattttcttccattgacccatatttatacctttcaggtactgaagatttaataataataaatgtaaatactgtgaagtgtgtgtgattttacaatggacttatggttggtgggaaaattcagcatggaaatgcttttcaaaatatgatagcggtcattattttgattgtgccttactgaaagtttttggggaatttacaagagtactgattacatgattatctggagaaaataagatgtctttgaaatacgtgttggcttcaagaaaacagttttaacgttttcctaaaatgaaatcttttgaggtgagcttatggcatcaacacatggttgatgaggaagctgagttgcattagtgcacatgatttccagtcaggtcatgggaaatgaacagagacagtgacatctttgtagctgctcctttgtgaggcacttctttcttgagacgactccatgcacaaatataacagggatcattgggaatgacaccatcacagccaccaagtttattggtttactgataaatcaattttgagggttttatgctagaaaagataacattataaatctttgtttttcaacagtggaaactgaggtgcttcagttttgtttttcataaacatctatctcatggtaaaatgacgtatagtagcaatcatatgatagaaagaacacattggtgctcattttcataaacttcattttgttagattgtcacttagcaaagactattgcggattcttccttgagaactttcctttcaattttctgttgatttccttaaaattttcttcagggtcgagcaagttctgttgatatattgtgtcatgcaaagaaaactgtatttgtggttagaaaatgaagattttagacctcttattctgatttacttattaggcaagtcactttacttcatgaaacctatttcactgcatatgaagtagaaataattataatttcctcataatgttactgttagctgaaataaagtaaatgtgaaatca

SEQ ID NO: 300 - RNA, 2863 nt, Homo sapiens
  source 1..2863: mol_type = genomic RNA
gcatttctcctctgtgctcactgccacacacagctcaacctggacagcacagccagaggcgagatgcttctctgctgatctgagtctgcctgcagcatggacctgggtcttccctgaagcatctccagggctggagggacgactgccatgcaccgagggctcatccatccgcagagcagggcagtgggaggagacgccatgacccccatcgtcacagtcctgatctgtctcgggctgagtctgggccccaggacccgcgtgcagacagggaccatccccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagatgaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcatggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactagcccggagggtacgcagactccacactcagtagaaggagactcaggactgctgaaggcacgggagctgcccccagtggacaccaatgaaccccagtcagcctggacccctaacaaagaccatgaggagatgctgggaactttgggactcacttgattctgcagtcgaaataactaatatccctacattttttaattaaagcaacagacttctcaataatcaatgagttaaccgagaaaactaaaatcagaagtaagaatgtgctttaaactgaatcacaatataaatattacacatcacacaatgaaattgaaaaagtacaaaccacaaatgaaaaaagtagaaacgaaaaaaaaaaactaggaaatgaatgacgttggctttcgtataaggaatttagaaaaagaataaccaattattccaaatgaaggtgtaagaaagggaataagaagaagaagagttgctcatgaggaaaaaccaaaacttgaaaattcaacaaagccaatgaagctcattcttgaaaatattaattacagtcataaatcctaactacattgagcaagagaaagaaagagcaggcacgcatttccatatgggagtgagccagcagacagcccagcagatcctacacacattttcacaaactaaccccagaacaggctgcaaacctataccaatatactagaaaatgcagattaaatggatgaaatattcaaaactggagtttacataatgaacgtaagagtaatcagagaatctgactcattttaaatgtgtgtgtatgtgtgtgtatatatatgtgtgtgtgtgtgtgtgtgtgtgtgtgtgaaaaacattgactgtaataaaaatgttcccatcgtatcaactcca

SEQ ID NO: 301 - RNA, 2095 nt, Homo sapiens
  source 1..2095: mol_type = genomic RNA
actgaggactcatccatctgcacagctggggcccctgggaggagacgccatgatccccaccttcacggctctgctctgcctcgggctgagtctgggccccaggacccacatgcaggcagggcccctccccaaacccaccctctgggctgagccaggctctgtgatcagctgggggaactctgtgaccatctggtgtcaggggaccctggaggctcgggagtaccgtctggataaagaggaaagcccagcaccctgggacagacagaacccactggagcccaagaacaaggccagattctccatcccatccatgacagaggactatgcagggagataccgctgttactatcgcagccctgtaggctggtcacagcccagtgaccccctggagctggtgatgacaggagcctacagtaaacccaccctttcagccctgccgagtcctcttgtgacctcaggaaagagcgtgaccctgctgtgtcagtcacggagcccaatggacacttttcttctgatcaaggagcgggcagcccatcccctactgcatctgagatcagagcacggagctcagcagcaccaggctgaattccccatgagtcctgtgacctcagtgcacggggggacctacaggtgcttcagctcacacggcttctcccactacctgctgtcacaccccagtgaccccctggagctcatagtctcaggatccttggagggtcccaggccctcacccacaaggtccgtctcaacagctgcaggccctgaggaccagcccctcatgcctacagggtcagtcccccacagtggtctgagaaggcactgggaggtactgatcggggtcttggtggtctccatcctgcttctctccctcctcctcttcctcctcctccaacactggcgtcagggaaaacacaggacattggcccagagacaggctgatttccaacgtcctccaggggctgccgagccagagcccaaggacgggggcctacagaggaggtccagcccagctgctgacgtccagggagaaaacttctgtgctgccgtgaagaacacacagcctgaggacggggtggaaatggacactcggcagagcccacacgatgaagacccccaggcagtgacgtatgccaaggtgaaacactccagacctaggagagaaatggcctctcctccctccccactgtctggggaattcctggacacaaaggacagacaggcagaagaggacagacagatggacactgaggctgctgcatctgaagccccccaggatgtgacctacgcccggctgcacagctttaccctcagacagaaggcaactgagcctcctccatcccaggaaggggcctctccagctgagcccagtgtctatgccactctggccatccactaatccaggggggacccagaccccacaagccatggagactcaggaccccagaaggcatggaagctgcctccagtagacatcactgaaccccagccagcccagacccctgacacagaccactagaagattccgggaacgttgggagtcacctgattctgcaaagataaataatatccctgcattatcaaaataaagtagcagacctctcaattcacaatgagttaactgataaaacaaaacagaagtcagacaatgttttaaattgaatgatcatgtaaatattacacatcaaaccaatgacatgggaaaatgggagcttctaatgaggacaaacaaaaaatagagaaaaattaataaagtcaaaatgtttattcttgaaaacattaatgatacatgaatcttggccacaatgagaaaaataaaaatgaaaaaagagcaggcatccatttccatacaggaacaaaataggaggcagcactacagaccctacacacagctttacagaggtgaaagaaaactgtcagcaattctatgctgacataacagaaaatgtagatgagatagatgaaatacgaaaaattacagtttacttaatgaacataaggataaatagaaaaactgaatcatcatacataaacatatataaaatgcattgatcctgtaatcaaaaatgttcccacaaagtaaatgccacttcagcaa

SEQ ID NO: 302 - RNA, 2237 nt, Homo sapiens
  source 1..2237: mol_type = genomic RNA
atttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgtt

SEQ ID NO: 303 - RNA, 3060 nt, Homo sapiens
  source 1..3060: mol_type = genomic RNA
actcacagcacgaccagagaacaggcctgtctcaggcaggccctgcgcctcctatgcggagatgctactgccactgctgctgtcctcgctgctgggcgggtcccaggctatggatgggagattctggatacgagtgcaggagtcagtgatggtgccggagggcctgtgcatctctgtgccctgctctttctcctacccccgacaggactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcaacggcattctccaacggagcgtttctgggaatcggcatcacggctcttcttttcctctgcctggccctgatcatcatgaagattctaccgaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagtttccttctctccctctctctctctctttctctctctctctctctttctctctcttttaaaaaaacatctggccagggcacagtggctcacgcctgtaatcccagcactttgggaggttgaggtgggcagatcgcctgaggtcgggagttcgagaccagcctggccaacttggtgaaaccccgtctctactaaaaatacaaaaattagctgggcatggtggcaggcgcctgtaatcctacctacttgggaagctgaggcaggagaatcacttgaacctgggagacggaggttgcagtgagccaagatcacaccattgcacgccagcctgggcaacaaagcgagactccatctcaaaaaaaaaatcctccaaatgggttgggtgtctgtaatcccagcactttgggaggctaaggtgggtggattgcttgagcccaggagttcgagaccagcctgggcaacatggtgaaaccccatctctacaaaaaatacaaaacatagctgggcttggtggtgtgtgcctgtagtcccagctgtcagacatttaaaccagagcaactccatctggaataggagctgaataaaatgaggctgagacctactgggctgcattctcagacagtggaggcattctaagtcacaggatgagacaggaggtccgtacaagatacaggtcataaagactttgctgataaaacagattgcagtaaagaagccaaccaaatcccaccaaaaccaagttggccacgagagtgacctctggtcgtcctcactgctacactcctgacagcaccatgacagtttacaaatgccatggcaacatcaggaagttacccgatatgtcccaaaagggggaggaatgaataatccaccccttgtttagcaaataagcaagaaataaccataaaagtgggcaaccagcagctctaggcgctgctcttgtctatggagtagccattcttttgttcctttactttcttaataaacttgctttcaccttac

SEQ ID NO: 304 - RNA, 789 nt, Homo sapiens
  source 1..789: mol_type = genomic RNA
actctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccagggctgagagacacgtgaaggaagatgatgggaggaaaagcccaggagaagtcccaccagggaccagcccagcctgcatacttgccacttggccaccaggactccttgttctgctctggcaagagactactctgcctgaacactgcttctcctggaccctggaagcagggactggttgagggagtggggaggtggtaagaacacctgacaacttctgaatattggacattttaaacacttacaaataaatccaagactgtcatatttagctgga

SEQ ID NO: 305 - RNA, 4899 nt, Homo sapiens
  source 1..4899: mol_type = genomic RNA
gtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtc

SEQ ID NO: 306 - RNA, 3982 nt, Homo sapiens
  source 1..3982: mol_type = genomic RNA
ggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggtgaggggttgcctccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacaccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctgaaaa

SEQ ID NO: 307 - RNA, 1747 nt, Homo sapiens
  source 1..1747: mol_type = genomic RNA
agcctgacttcagcgctcccactctcggccgacacccctcatggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtgacaaagtgactgcccggtcagatcagctggatctcagcggggagccacgtctcttgcactgtggtctctgcatggaccccagggctgtggggacttgggggacagtaatcaagtaatccccttttccagaatgcattaacccactcccctgacctcacgctggggcaggtccccaagtgtgcaagctcagtattcatgatggtgggggatggagtgtcttccgaggttcttgggggaaaaaaaattgtagcatatttaagggaggcaatgaaccctctcccccacctcttccctgcccaaatctgtctcctagaatcttatgtgctgtgaataataggccttcactgcccctccagtttttatagacctgaggttccagtgtctcctggtaactggaacctctcctgagggggaatcctggtgctcaaattaccctccaaaagcaagtagccaaagccgttgccaaaccccacccataaatcaatgggccctttatttatgacgactttatttattctaatatgattttatagtatttatatatattgggtcgtctgcttcccttgtatttttcttcctttttttgtaatattgaaaacgacgatataattattataagtagactataatatatttagtaatatatattattaccttaaaagtctatttttgtgttttgggcatttttaaataaacaatctgagtgtaa

SEQ ID NO: 308 - RNA, 7059 nt, Homo sapiens
  source 1..7059: mol_type = genomic RNA
gcaacacttcctctgcatctggatatgaagggagccccagaaaagcggaagaatttagacgcacactgggtaggtttgaatttgttttgttttcaaaaattaaacaaatgatccttcagcatcatcgcctccgctgctttatcaggtcgcatagggcatggagctggagaactataaacagcccgtggtgctgagagaggacaactgccgaaggcgccggaggatgaagccgcgcagtgctgcggccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccactggtggatgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcagcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctaatactttaggctagaatcaaaaacaagttagtgttctatggtttaaattagcatagcaatcatcgaacttggatttcaaatgcaatagacattgtgaaagctggcatttcagaagtatagctcttttcctacctgaactcttccctggagaaaagatgttggcattgctgattgtttggttaagcaatgtccagtgctaggattatttgcaggtttggttttttctcatttgtctgtggcattggagaatattctcggtttaaacagactaatgacttccttattgtccctgatattttgactatcttactattgagtgcttctggaaattctttggaataattgatgacatctattttcatctgggtttagtctcaattttggttatctttgtgttcctcaagctctttaaagaaaaagatgtaatcgttgtaacctttgtctcattccttaaatgatgcttccaaacatctccttagtgtctgcaggtgttagtggtgtgctaaaagcaaggaaagcgagttagtcttttcagtgtcttttgcaattcaattcttttgtcatgtataactgagacacacaaacacagcaggagaaatctaaaccgttgtgccttgaccttcctctgctggtcttgttccagggttatgaatatgaaaaaatagagatgagactttttgtgtcaactctgtccacaagagtgagttatctagtatgattagtatagctttctccagcatggcagcaggaagtaactacagggcctcttttatgcctgacatttcttcccttcctttttccctgcctccctttttcatcaattgcgatgctcccacaactctttacagacttgtgaaatcttcaagaacacctttactctataactcaaaaattagttgaaaaataattacttctcaaggattattagaatcttaggtacttatttgtaaagatgtttagtgacttttttttcaagtatcttattaaaggaggcattctagaaaatatgaattagtttccaaatgccttaattttaaactttggcctgaacagttttttctttttcttaatggaagaagatatttaatatcttaaaaatattccaagttaggaagaacactacttgccttatccatttcccatttaaaggacttttaaactttgacacatccttcagatttcctgaaaataattgaaatatcttactttaaaaatattttcatctctgaaatatctcgttatttattggaggtattgtttaaccttagagagaccattaaattatttataaaatattttgtaattacctgtagctaatacattacatagaaaaaaactatgttaacagtgtctctgtttaagtataatcagatataaatatatacttaattttttaattttaaaaatagatacctgtttgactttgaggtagtccagaccttttctttttttttttttttttaatgtgtgcaaaagcccaaaggttcctaagcctggctgcaaagaagaatcaacagggacactttttaaaaacactcttatcagcctgggcaacacagtgagactccatctcttaaaaaaaaaattagctgggtatagtggtatgtgcctgtagtcccaggtactcaggaggctgaggcaggaggattgcctgagcccaggaggtggaaactgcagagagtcatgatcatgtccttacactccagcctggataacagagcgagaccctgtctcaaaaaaataaaataaaaaataaaaacacccttgcctgcgctccattcccaggttataatttaattactgtgggatgagacccagacatcaatatttttctagattcttcaggtgattctaattcacagccagagttggaagccctgcgtggcctttgaaggtctagatgattcttcttccttgccctttgagcttttccccatctcacaggtatctagaaaaaaactcctcttctttggcaacctgtccttttaaaatcacactctacccacctgtacagaagaccatgccctataatgaaatgtttattcctatctataaatggaggataaacattttgtggcacttctggaccaactattccctactattcttttgaagaaaggcaggaagagtactttctaattcagaagaggatgttttcactattctgataaacaatagccaagttcagaccttgtacagattctttttatttgaattgctgaaataatttattgatgatgaaaaaaagattagagaggaatacatttatatttagcttattggcacatgtgcatacatatttcctcttcaaatgaaccagttctttcatttcattatgctaatatatatatataacatatatatgctaatatatatataacatatatatgctaatatatatatatcacacatatatatcacagttttatatatatatatgtatgtgtgtgtgtatatatatatatatatatcacagttgggcttgattcttccgtattccaaagagcataatttcagttctatagacttatagataaataaaaatcatctttgtgggcttccttcctttactgttcgcagtgaattacatgacgaacaacttctatacctttgaaaatgttctagaactagaatcatcctgctactgggaactacccacagctctatcttcaatgccaggtgaaaacacagatcacaagtcagatgaatcaggccaaagcaacttttatgtatatctaggactggcgacataatttgcagagcccagtgaacagtgaaaatgcagtcccatcattcaaaaattattaagaaattcgagatggcaacagcagttgtcacaccaagcacagtgcccttctgagcatgaggctctgtgtgactgcatgggtccatgagaccagcactgtgtgggtctgaatgatggctttggtgttcagtttagcacacgcggtctaccacgtctgcatgagtggtaaatgtagtgcctgggtcaaaatgttccttctgttcaaatcgaaatacctcatctctatggctctatggctgtacattaggacctagaacagtggcccattgctcttagactggaaccatgtccactaaaataaacctaagcagatgttgtagacctagccccacaggactgcatttagctgcttcagtgacactttgatgaaagtatggagaagtggagacattatagataaaatatatcaattcccagagaaaactcttgacttaaaaacttaactgtagtaaatatatctttttcaggtgatgaattatttttttaaaaaaggttacatataggaattctgcagtataatttggaggctattagtgctatattaatggaaattaattattttttaagtaagtccaaaaaataatctagaaagtaagtttccagagcaaatctgacctagcatttggtatgctaggctctgcttttcatgattttgaaataaatcataattagacttaacaatatggagaaaataaacttgtatttttaagtgttctgttggcttattttctgtttcatccaactcaataattctgataaataaatttggttctagtttggtgcttgtc

SEQ ID NO: 309 - RNA, 3088 nt, Homo sapiens
  source 1..3088: mol_type = genomic RNA
attctgagcctagccacagaggtaaactccggacatttttttctttcatgtcgccaaatacatccttgtcattaaaagacctttgaaactgagtaaaatggctttgaaacctcagtagaatatgctcaaactgtctggagaaggattacgggacagctaccacagtagaagagatcaaattgcccttaaaaatctccagtctgatgtcacagaggcaaaatctgacttcaccaaggagaccctggcatcacaaaacacaaaaatgatttcatccatagtcatctcccagatgattgatgagaataagtcaagagaaaacagggcctccttgcccctaccgtgtgccattgctcagtctcgtgcacatcacgccaagcaatctctggctaaccgcagcggagtcaacattcacagagcatttgcgttacttccgggcagattaggaatcccggcaccgtcagatgagcgaggacctgaggcagagctgccgcccaaagaggagagaccctgtgggggcccccgcagagggtttgcatccatcaccatcacggccagacgcgtgggccccccagcccgcgccctggtgtgggggactgctggggactctctgtgccccaagtgcagggctgaggacacactgttccaggcgcccccggctctggccaatggcgcccatccaggtcggcatcagagatcttttgcctgcacagagttctccagaaacagctccgtggtgcggctgaaggttcccgaggcccacactgggttgtgtgagagacgcaagtactgggtcacccatgcagacgacaaagagaccagtttttctccagacaccccactgtcaggaaagagcccgctggtgttcagttcctgtgtccacctcagggtgtctcagcagtgtccagattcaatctattacgtagacaagtctctctccgtccccattgagccacctcaaattgccagccccaaaatgcacagatccgtcctgtcgctcaacctcaattgtagttcccacagattaacagcagatggagtagatggcctagtgaacagagagccaataagcgaagccctgaagcaggagctcctggagggagaccaggacctcgtaggccagcgctggaacccaggtttacaagaaagtcacttgaaggaaaccccatcgttgaggcgggtgcatttggggaccggcgcttgtccttggagtggttcttttccattggaaaacacagaattggcaaatgtgggagctaaccaagtcactgtaagaaaaggggaaaaggaccatacaactcattgtcacgccagcgatcatgccaaccaactgtccattcacattcctggctggagttacagggcagtacacacaaaagtattttctggaagcagcaagaggcaacaaggagaagtatgtatgactgtgtctgctcctccagtggaacagaagcccactagacatttccttcccattggggatagctctccaagcgatgactgtctgtctagagacctttctgagcccacagagagacgacatcaaagcttcctgaaacccagaatcctttttcctgggtttctttgccccttacaagatgtatgtgcatctctgcaggaagacaatggtgttcagatagaaagcaagttccccaaaggagattacacatgctgtgacttggttgtaaaaataaaggaatgtaagaagagtgaggaccccaccacacctgagccctccccagcagcaccctcgccagcaccccgcgatggagcagggagccctggcctgtccgaagactgttctgagtctcagcaaacgcctgcaagatctttgaccttgcaagaagcactggaagttcgtaagcctcagttcatttctcgctcacaagaacggctgaagaagcttgaacacatggtccagcagaggaaagcccagcggaaggaggacctgaggcagaagcagagcctccttcccatccgcaccagcaagaagcagttcacgattccccatcctctgagtgataacttgttcaaacccaaagaacggtgcatttcagagaaggagatgcatatgcgatctaagagaatctatgataacttgccggaagttaaaaagaaaaaagaagaacaaaggaaaagggtgatcttacaaagtaacagactccgtgccgaagtcttcaaaaagcaattactggaccagctccttcaaaggaatgcggtctaaccacaggctgccctgctgaccacactacctggacctgggaggacttcgaatgctggataagccattaaacttagcattatcttcatcatgggaaaacacttattttactttcgattttttttttccataaaggaaaacatcacttcctgaattactgacccaagccaaacagaaacaataaccctcgagaaaataggtcaacaatctgttgtggggaaaccacctccatgtaacccactggatacagtccccttaatgtttttgcttctaaattgtaccttttgcttctgatttcttctcccctgctgtttcctgcccatcagagaggcctgatacaagcaagtttgtttacatccctggggaatcttttacatcaaacttttgggatccaaatccatctccttttaaatttcaatctcagcacctgccagttatgcaaatgccaaaatgtgggtcatcatatagtatatttgaaacctttctgaacatgtacaccacccaatgctagaggctgacttggaaaccggtgggtgcaatgcccgaggctgtggaacaatcagcccatctctttgtgacctggagcagtcagagggccctcagtcaccccgccccattcctcaccagagaaaagaatccatttctatggaagctctgacgtaacttcagtgttttctacaatactcctcctgccccgccccattaaaacagttcttttgttaagaaatagcctaatggtccaactttgctgtctgttcttccaaatgtttataatacacattatttataaatatgtctgtttgggaagctaagaacaagctagtttttacaacacaaatggaaataaatgcaattattataaaaattcaa

SEQ ID NO: 310 - RNA, 4510 nt, Homo sapiens
  source 1..4510: mol_type = genomic RNA
aattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactatttaaaaattgacagatcttttctgaagataaactttgattgtttctata

SEQ ID NO: 311 - RNA, 10034 nt, Homo sapiens
  source 1..10034: mol_type = genomic RNA
gtggacgcggcggccccgcggcgggggcagcgggcggcgggggcggaggcggccgctagcgccctgcccggcgccgcctccttcggcgttcgccccacggaccggcaggcggcggaccgcggcccaggctgaagctcagggccctgtctgctctgtggactcaacagtttgtggcaagacaagctcagaactgagaagctgtcaccacagtagcttggagctcggcggcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggtaggtggctaatctcaggactgggaagattacggcgaattatgctcaatggtctgattttaactcacccgatgttaatcaatgcacattgctttagatcacattcgtgatttaccatttaattaactggtaacctcaaaattcgtggcctgtcttcccattcaccccgcttttgactattgtgctcctttataattctgaaaactaatcagcactttttaacaatgtttataatcctataagtctagatgtatccaaaggtgaagtatgtaaaaagcagcaaaatatttatttcaaagacttcacttctgtttcctgaatctaaagaaagacaacatgctgctttttaatcataggatggagaattttaaagaactgtttgggccaggcacagtcgctcatacttgtaatcccagcactttgggaggccgaggcgggtggatcacaaggtcagcagatcgagaccatcctggccaacatggtgaaaccctgtctctactaaaaatacaaaaattagccgggtgtggtggcacatgcctgtaatcccagctactcgggaagctgaggcaggagaattgcttgaaccagggagttggaggttgcagtgagctaagactgcaccactgcactccagcctggtgacagaacgagactctgtcttaaaaacaaacaaacaaaaaaaaaatctgttagataagctatcaaaatgcagctgttgttttgtttttggctcactgttttcgtggttgtaactaatatgtggaaaggcccatttccaggtttgcgtagaagagcccagaaaacagagtctcaagacccccgctctggactgtcataagctagcacccgtggtaagcgggacgagacaagctcccgaagcccgccagcttcctgctccactcagctccgtccagtcaacctgaacccacccagtccagctgtctgtgggaatggtggtgttcttagggacagactgacaccttacttgtcagtgttcctccgggccccatttggcagctcccgtatcttttgttatgttgcttttaaagatatgatgttttattgttttaactcttggtgacagtagatgctctctggagcgcagacgaggcacatgtgtcttcatagcctgggctgggtgggagccagtcaccctgcggatcgagagagggggtagagtcttcttcaaatggcagttttacttcaaatggcagatttcacaagagttggttattttttacaatggtttaggttgttaagtctcctttgtatgtaaggtagttttttcaacatctaaaatttttgttttagccttcaaaaccaacttaccaacctcagtccagctgggaaggcagcgttgattatggtagtttgtcaagaatatatggacctggaaacactttctctctctgtccacctggtagataaattgtcctgttgagaatttttagatctggactggaactgccaggaccaccgcctccagggagtcgctgggcacctggaggtatcgtcgatgcctctcccccatctttagaaaatttggctcttctgaggtcattattattttaagaatgattaggattgataagggtcccatgaccagcattatgaaaatgcgagagtgggaaggacacagtgtgagacttccactagaaaaaagtgaaagttagggttaggacatccttttttaaaaattacaaatttagtccgttttggtttttgtaatcaggctaggcacagtggctcacacatggaatcccagcactttgggaggccgaggtgggaggatcacttgagcccaggagttcgagaccagcctaggcaacatagcaagaccctgtctgtacacaaaatttaaaaattagttcatcggggtggcacacatcagtagtcccagctactctgcaggctgaggtgggaggattgcttgaacccaggaggtcgaggctgcagtgagctgtgatctcaccactgcattccagcctgggtgacagagttagattccaccctctcccaccccggcaaaaaaaaaaaaaaaagatgcaatcaaaggggctgttggccagcaatggcagcagcagcggcgggcagtctgcccaagtgtcttaggaaccaaaagcaaataaaagtgtttccatatatgccaccagccaagtggccatcctaattcagaaagaagctagcctttgagtgtctgtcatggtgcatccgtttcagtattatttcctaaaatgagaagcccctgtgtcaacaagatccaggggctggagcccaatgccaagcctgtgttgtccccagcgaccctgcagctgctcgctctgatgtaccctgtgccattcaaggagatgtggtccaggaaagtgagcctcatggttttcagagaagtcattgttctgtttacattttcataaaacctgtttaaaatagctccccgtctcaggctttcagcagtaacagtgagctgactggcaagttcgatgttagctcccgggacactcagcagcgatggtgagcattttggtttccttaaggcccagcaagacttccagggacatctctggtgaagccagaatggagacacccgtgacctcaggctgaaagtcactcgacattggtctcttgtgttgatagggaaggaaatcaggcattcctatttctttaaataacaaaaccactaattgccactcaatgctggaatattttgggtcacctaatcatagatttctcagggcatcaatactcaaatataggctgattatgccccagttcaaatgggaactattaacagagtgcatttcttgcttgctgggtttcaacagacatcagccaaaagaacaaaagagatgtcaggacagattccaggagtgtcggagcacatgtgtggcacccgctccctctggcagcgaatgtaggaagtcgccaaatttacccactcttcaacaagtcattgtttaaacacggtttttcattttctcaacttttaatagcaaaaagtgccaaagtcctcagagacctaacagccttggtctaccgtgctgaccagggtgaaggcacggcgagggactcctcccagacgtgcctcttgtgtgccagctggctgtggctcgggagcagacgcaggcctctccattgtccaggggagcctggcggcgcatccctcctctcccacctcctggcacttccagctgggtgtcccacatgttggattccgtccccaccacacttccagagaccggagaactgtgcagggcctaaggccgtttggatgaattgtcaaaacaagatgcttccagttacagcggcaggagcgggactgggagcacgggctgacggctgctggtgcctttcttcccacctcgcttgcctgtttccgcttgacccttcctccagctccgatgagaagagtataaagcatcttcctaacgggtgtgtttgctatacgaacataatggacgtgaagtggggcagaaacccagaactcagcattcaaggatgcccaggagagctgtccctgttttaaagagctgtgttttgttttgtttcgcatttagagagcagacaaggcacccttctgctgcgctgatacgtttcttacactgggccattttagacccccagggaaacagccttcctggagcgttgtctggaggttccagggacagggcagcctcccagagccgagcaagagctcaaggtacaaatgagagatttgctataccgtgagaagtcaacaacttagccaccacttccccgcaatggaccatgtaacaaatacctcagcaggccctgcaaaaggccatgctagagctgaggcgcacagcctgtggcctctgtagttagggcaggtgggatggagactccttgagtgcacacacctgagcctgcccacacacaggggagcagcatctcgtatgacgtctggaaggaacttcggttgtgtaaagggagccttgaagatacgtgcaaaaggtgctaccccaatttggtgaaactgacattgggcacgtcttgggcttaggagaagcggccgatggtcccggcctgcagtgacaaacccccctccccgcaccgcccccagcaccccctctcctcttcacctcttcctgctggccacgaggaagccacttcctcagagagaccctaccagatgcggatggaaacagatgcaccaaagcaagccctgatgaaaccgcgacttcctaaggtctgtctcctctgaacttgcacctgggcctctctgtgtttggttccaagcacttcccacctcaaactcccattttcaaaccactgtatctctgcgcacatctgctacttaccagccgcatacatgatggagggttttttggtcctgatccagtggccacacctgtctttgaaatgtctcactgaactccagttttaaaatagattcattgcttcaacacagcaagcccaatgcacccagctaagactggcttgaccgacagcctggcctttggtggggggcttcctggggcctggggaaagctggccaccttcaacagctggtacctcttcaacagtgtggcctttcaaaatgcagatgccaccaggagaacatgcccacagctcaccacctatggatgccatggctctgggcagctttcaaagcaggttcctgtggtctcctcagctgtttgagggggtaacagcaaatcagcctccattttaaaatgaaaacaccagcctccagatgtagggcctgctgggtgttgctagccgctggtccccaggcacggtgcactttctccacctcctgcagcctccctgttgtttctagactcttgcacctggtgagtgcaaggataggtgacccaggggcctgcagccttgtcctcagctcccatctcctggactgccagcctcaccctctgcagttagcatggttggcctgatgcagggatcccgagggattactttttagaccttctttcacattcagaaaagtagtatagattcaggagaggcaagaaaattatgctgtccatagaagtcacccatgaagactgatgccaccacctgaaggctcatgattgttaaaaatgtccacgggaacctctcgtccacaggaggtttgtctcaacacttcccatttttacggcattggcattgccaagcatggggaagtatctgctcttctcatgttaaaagtggcccagcttttcttaactcagtccaagctgacttgtttagctgcactggaatttcttaccaaccaaatatttgcatcgagcaaagggggctgtgtgcacctccctaatggcagcgatgatggctgctgtcattcaagcccatcttcagacgtcacagtctggaagtgaaatgtccacaaacatctgtggcagaaaaggctatacggaccacccagttgtgctgcagctttacagagcaaggaagggttgtggcaaataaatgattaacctgcctcgactgtgctgagggcaacaaaggccatctcaccaaaggattattcgatgccattaaatcatcccgtgaccttcctgcttccgagtccatggcctttgcccagggcatgtactcccctgagaggccttctgcctagaaagatctatgactgggttccaaagttgaggcctaggtttttgctgggatttagatattttcaggcaccattttgacagcattcaggaaaacggttattgaccccatagactagggtaagaataaaggcaataaatttggtctgactcagaatataggagatccatatatttctctggaaaccacagtgtacactaaaatgtgaaattgaaggttttgttaaaaagaaaaagataatgagcttcatgctttgtttaattacataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtgaggggagaggagctatggtatgtggttcagctgaaacacatacaactgcatccttttggagtcctttgccaacaaaaacagaccaacagaccagatggtgtccatgttcaatatcatgtcttgatggacgcagctgatgacctcaaatacttgagtggtctcatggctgttagatggattatttgaaaaaaaaaaaaaaaaaagagagaaaaaataattgatttttacatcagagatagcaaactaagacctggggaggggggtcagcttttattttattttattttttttaagtttgctagttgggtcaaatgtgaggaggagggagtctacctgccacctcttctcttgcccctcttctgcccacacatccagcatccaaaatccattcatttaatgaattgataaagtgccgtgcaaactggtgcacaaacaggcccccagtccacgcagcctggctcctaggaaaagtggtgaccgggcgtgggggggcatgccgcagccctgggacacagtcgggcaccttccccggacccccaggccttggctgtgcctcaagtcagagagggtcagccttcaggccccggagacgagtgactggccgatcatttcacaataaaatcactcacttttggcaacttcactttttttaaggcacagtcagttccttttctcatgtacctcacaaaagatgaagaccatgtagtactctttttggtaaagttacagtgttcatgttaaatatcacttttttctacattgtgtggtaaaaagaactacgttaatagctatatcttaaatactgtgatttgactttttgaaaaatatcctaatacaaatattttactaacttacaatcactcatttaataagaaacatttggattcttttgaaatcagtgttaattgactcatattcttaaaagcctggctcttgaccctattggaaacacaaaggaagctgaaatcaaacatctaaaatacactgcgtacacgtgtgcgtgcacacacacacacacacacacacacacacagctcttcatttctcctgagccatgcagaatttactttcaatgtggaaatctgttccctttaccacactgtatatgcacagagcacaagagaggctatctctagtcacttccaccagcgaggccttagactccgtattagaggccaccgatttcatacaacagtgtttcgctaaagacccttcactattcttgtttagtaaatagctgtctgctcttcagggaactgttacctatgggttattaccaaagaacgctggcaattggaaatgtcctgatggaaattctttgcacgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaattt

SEQ ID NO: 312 - RNA, 1323 nt, Homo sapiens
  source 1..1323: mol_type = genomic RNA
gccttaacctctgctggtgaccagaagcctgcatttctgcattctgcttaattccctttccttagatttgaaagaagccaacactaaaccacaaatatacaacaaggccattttctcaaacgagagtcagcctttaacgaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaa

SEQ ID NO: 313 - RNA, 3038 nt, Homo sapiens
  source 1..3038: mol_type = genomic RNA
ggcagtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaaggtcagtcccggggatttgtagctcgggctggggagccctgtgtgtgctggccccacttcgggacacacgggatgatgctcaccccaccttcttcacccctagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaa

SEQ ID NO: 314 - RNA, 4464 nt, Homo sapiens
  source 1..4464: mol_type = genomic RNA
gcagtcagtccggtgtttgcaaatattgtgtgggctccgcgcgctgcgggctgcgggagggtccggccgggcgtctctgcgagcctggagtttgcatgaaactttcacctgcgctccggggagactttcggctccggctcccaccgcgcgcctcgccgccctcgcgaccgcgggctccgtccaacccggcccgacatggacgtgctccccatgtgcagcatcttccaggagctccagatcgtgcacgagaccggctacttctcggcgctgccgtctctggaggagtactggcaacagacctgcctagagctggaacgttacctccagagcgagccctgctatgtttcagcctcagaaatcaaatttgacagccaggaagatctgtggaccaaaatcattctggctcgggagaaaaaggaggaatccgaactgaagatatcttccagtcctccagaggacactctcatcagcccgagcttttgttacaacttagagaccaacagcctgaactcagatgtcagcagcgaatcctctgacagctccgaggaactttctcccacggccaagtttacctccgaccccattggcgaagttttggtcagctcgggaaaattgagctcctctgtcacctccacgcctccatcttctccggaactgagcagggaaccttctcaactgtggggttgcgtgcccggggagctgccctcgccagggaaggtgcgcagcgggacttcggggaagccaggagaaaagccttacagatgctcatgggaagggtgtgagtggcgttttgcaagaagtgatgagttaaccaggcacttccgaaagcacaccggggccaagccttttaaatgctcccactgtgacaggtgtttttccaggtctgaccacctggccctgcacatgaagaggcacctctgagggagcagagaggtggatcctgtaggctaaaaggcttccaggctgagagccggccgtggaaggagggatgcgtgttccagccaaagcatgccgttctgcaccctacccagttgcctccagggcctctccttggaaggtcttttgagggctaaaaaggtcctgtaagaagcggcatagcacccgtggtgcatggtatgtgggtgaccctggactcgccactggtacccgcccttccgagcggcgcctaagcctttgccgtgagcatgcacactgagaatgctaatggttgggttgattgtatgttgaggatctattactgaccgtatgatgaggccaactttttttccttgtggttagcaagactgcaagagatggaaaaaaagtagtttgaatgttttgtgtgtaaggagtataccatgagatgagatgaccaccaatcatttccttggggggagggggtgtctgcaccttagaaaaaaaaagaaaaatcaaaaaaacaaaaaaacaaaaacaaaaaaagaaggaaaatcttggagggtgggcgtgggaactcaggaccccagagtggcgagtggtgtggggagggagagcctctctcccccttttctgtgtgagaggaactcttagtgtctggtgcagctattaaatgtgcaatgtgtcaagtagcttgttttacacgctacaacatagctcatttgtaacccattgtataagctgtgtatttacaaatataacacaacaatttaacttttccttagaatacaaaaagtcatgcatggtctggggaactatatgcttttccatttttaagtcaggactgcaatactgattccagttaatgagcagctaagatccaatctgtctaatacagtgaccccctagccatccgggcctggcaatatacaattttttttcccctccaagtttgtaacactccccttccagaaaggcattgtgcaacacaggattatttttaaatgattctgaatttgaattaactttttggagaattctgtgatgcccttagaagaaattggacacgtattgagtgtcacaaagctggggctgggaattgctggtctaatgtttcattagacttaagaacctaaaatttttctcagttgggtggataaaaccactaacgcttagaaactgttttctcatgcagctatgtttctcttatttatgccttgaggactaatttctggttttctagctgttaatgcactgttgaccttcataatggtgccttacgcaagcgatcccttctgtgggggtctcatacaggggtgtgggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctggaatggggcaccttcggccgttcagaagtggcctgaacagaatgctggaacccaggctggactcggacacactaaggttttgattttgaatttcagccttattagaagatctaacctaagagtaagctaaccacagggattcttttgtagaacactttttatgcagatgaagctattttttccagcaagtagattcttccagtttttccaaggagtaatttccccgaattggcataccacggcgtggacagctgatatttcacccagctgctggcttgtgggtgtggctctttgctttatatatatatacacacatgtgagtctggctgggctggtattttgtttgatcttcctggaaatgagcagtgactaacgctcacataactggttttttttttatctgggctgatgaatacatttacctaagaaactcatttcgttttacttaagaggggaagtgcagttttcttttggcagttcagaatccaagcacttgatttgctgggtttggaaaactccttttttggccttctatgtgcttagccataacaattccattaagcaagaaggtaagcaaaagacaaaaaaaaaaaaaggaaaaaaaaaaacttgcactggcttgtctcacttacgaaacatgtcggagctgtttgcctgggtggggctgggtaccgtacctgtcaatgcctgtgattttcataattagcacgtacataaagaagtacattctgttcaggtgataactgagcctcaatcaagcagaaactttttgcttgaaattaaaaaaaaatttctattagtgaaatttcttttttttttttttttggaagcaccctgttatctaaagaatctttgtaagatttttgtaaaattttgttttacaagattttatttgaaattgttttttgcaagattgttatatttctgtatgaatgtattttttattggaataacataaaagaattcttatcagcatcttgagtctggttgttttttttgggggaagggggttgttggaacagattcctcctgcattcatcaccctggcttcctcctgggggcaaatcttcattgagcaaccctgagaacaactcaccgacctcagccccttcctctttccacagcctgtcctgggagctggagaggatgtcagcgagcctgacattgccctccctgaatgcatcaaatactcttctccaaggactgacaaaaacaaccccccggctgtgggcacagtagggaaccatgccggtttgatcttccattgctcaagccagggaatgcattgcagaggcacctgcacaacgacaaacttgagtgtttccctgccacttctgtgcctccacagcctgctccagttccgcagctagagctgggcctaccttccccagccagccctgccacacacctggctgaggcatgtctgggaggggtggaggaggcagagggactccaggccagagggctgttctcacaacctcagcccactctcagtcaaggagggcagaaaataaaagatgacatcactgcca

SEQ ID NO: 315 - RNA, 2601 nt, Homo sapiens
  source 1..2601: mol_type = genomic RNA
attcgcgacccggaggtgcgcgggcgcgggcgagcagggtctccgggtgggcggcggcgacgccccgcgcaggctggaggccgccgaggctcgccatgccgggagaactctaactcccccatggagtcggccgacttctacgaggcggagccgcggcccccgatgagcagccacctgcagagccccccgcacgcgcccagcagcgccgccttcggctttccccggggcgcgggccccgcgcagcctcccgccccacctgccgccccggagccgctgggcggcatctgcgagcacgagacgtccatcgacatcagcgcctacatcgacccggccgccttcaacgacgagttcctggccgacctgttccagcacagccggcagcaggagaaggccaaggcggccgtgggccccacgggcggcggcggcggcggcgactttgactacccgggcgcgcccgcgggccccggcggcgccgtcatgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcgtgaggcgcgcggctgtgggaccgccctgggccagcctccggcggggacccagggagtggtttggggtcgccggatctcgaggcttgcccgagccgtgcgagccaggactaggagattccggtgcctcctgaaagcctggcctgctccgcgtgtcccctcccttcctctgcgccggacttggtgcgtctaagatgagggggccaggcggtggcttctccctgcgaggaggggagaattcttggggctgagctgggagcccggcaactctagtatttaggataaccttgtgccttggaaatgcaaactcaccgctccaatgcctactgagtagggggagcaaatcgtgccttgtcattttatttggaggtttcctgcctccttcccgaggctacagcagacccccatgagagaaggaggggagcaggcccgtggcaggaggagggctcagggagctgagatcccgacaagcccgccagccccagccgctcctccacgcctgtccttagaaaggggtggaaacatagggacttggggcttggaacctaaggttgttcccctagttctacatgaaggtggagggtctctagttccacgcctctcccacctccctccgcacacaccccaccccagcctgctataggctgggcttccccttggggcggaactcactgcgatgggggtcaccaggtgaccagtgggagcccccaccccgagtcacaccagaaagctaggtcgtgggtcagctctgaggatgtatacccctggtgggagagggagacctagagatctggctgtggggcgggcatggggggtgaagggccactgggaccctcagccttgtttgtactgtatgccttcagcattgcctaggaacacgaagcacgatcagtccatcccagagggaccggagttatgacaagctttccaaatattttgctttatcagccgatatcaacacttgtatctggcctctgtgccccagcagtgccttgtgcaatgtgaatgtgcgcgtctctgctaaaccaccattttatttggtttttgttttgttttggttttgctcggatacttgccaaaatgagactctccgtcggcagctgggggaagggtctgagactccctttccttttggttttgggattacttttgatcctgggggaccaatgaggtgaggggggttctcctttgccctcagctttccccagcccctccggcctgggctgcccacaaggcttgtcccccagaggccctggctcctggtcgggaagggaggtggcctcccgccaacgcatcactggggctgggagcagggaaggacggcttggttctcttcttttggggagaacgtagagtctcactctagatgttttatgtattatatctataatataaacatatcaaagtcaa

SEQ ID NO: 316 - RNA, 2113 nt, Homo sapiens
  source 1..2113: mol_type = genomic RNA
tcccaatcccggggcggccgggcgggggtgggcagggggcgtgaggccgcccctgcgtcccgggggccccccgaaaacgcgctccgggtgcccggtccctccgctgcgccctgccgccgtcctcccgggggtctcgggcggccgcggccgtgtccttcgcgtcccggcggcgcggcgggaggggccggcgtgacgcagcggttgctacgggccgcccttataaataaccgggctcaggagaaactttagcgagtcagagccgcgcacgggactgggaaggggacccacccgagggtccagccaccagccccctcactaatagcggccaccccggcagcggcggcagcagcagcagcgacgcagcggcgacagctcagagcagggaggccgcgccacctgcgggccggccggagcgggcagccccaggccccctccccgggcacccgcgttcatgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggccgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctagcgcggcccccgcgcgcgtccccctgccggccggggctgagactccggggagcgcccgcgcccgcgccctcgcccccgcccccggcggcgccggcaaaactttggcactggggcacttggcagcgcggggagcccgtcggtaattttaatattttattatatatatatatctatatttttgtccaaaccaaccgcacatgcagatggggctcccgcccgtggtgttatttaaagaagaaacgtctatgtgtacagatgaatgataaactctctgcttctccctctgcccctctccaggcgccggcgggcgggccggtttcgaagttgatgcaatcggtttaaacatggctgaacgcgtgtgtacacgggactgacgcaacccacgtgtaactgtcagccgggccctgagtaatcgcttaaagatgttcctacgggcttgttgctgttgatgttttgttttgttttgttttttggtctttttttgtattataaaaaataatctatttctatgagaaaagaggcgtctgtatattttgggaatcttttccgtttcaagcattaagaacacttttaataaacttttttttgagaatggttacaaagccttttgggggcagtaaaaaaa

SEQ ID NO: 317 - RNA, 4076 nt, Homo sapiens
  source 1..4076: mol_type = genomic RNA
ctcttcttcttaaacaaaccacaaacggatgtgagggaaggaaggtgtttcttttactcctgagcccagacacctcactctgttccgtctaagcttgttttgctgaacacttttttttaaaaaaggaaaaagaaaaggagttgcttgatgtgagagtgaaatggacgtaagattttatccacctccagcccagcccgccgctgcgcccgacgctccctgtctgggaccttctccctgcctggacccctactattgcaacaagtttgacggtgagaacatgtatatgagcatgacagagccgagccaggactatgtgccagccagccagtcctaccctggtccaagcctggaaagtgaagacttcaacattccaccaattactcctccttccctcccagaccactcgctggtgcacctgaatgaagttgagtctggttaccattctctgtgtcaccccatgaaccataatggcctgctaccatttcatccacaaaacatggacctccctgaaatcacagtctccaatatgctgggccaggatggaacactgctttctaattccatttctgtgatgccagatatacgaaacccagaaggaactcagtacagttcccatcctcagatggcagccatgagaccaaggggccagcctgcagacatcaggcagcagccaggaatgatgccacatggccagctgactaccattaaccagtcacagctaagtgctcaacttggtttgaatatgggaggaagcaatgttccccacaactcaccatctccacctggaagcaagtctgcaactccttcaccatccagttcagtgcatgaagatgaaggcgatgatacctctaagatcaatggtggagagaagcggcctgcctctgatatggggaaaaaaccaaaaactcccaaaaagaagaagaagaaggatcccaatgagccccagaagcctgtgtctgcctatgcgttattctttcgtgatactcaggccgccatcaagggccaaaatccaaacgctacctttggcgaagtctctaaaattgtggcttcaatgtgggacggtttaggagaagagcaaaaacaggtctataaaaagaaaaccgaggctgcgaagaaggagtacctgaagcaactcgcagcatacagagccagccttgtatccaagagctacagtgaacctgttgacgtgaagacatctcaacctcctcagctgatcaattcgaagccgtcggtgttccatgggcccagccaggcccactcggccctgtacctaagttcccactatcaccaacaaccgggaatgaatcctcacctaactgccatgcatcctagtctccccaggaacatagcccccaagccgaataaccaaatgccagtgactgtctctatagcaaacatggctgtgtcccctcctcctcccctccagatcagcccgcctcttcaccagcatctcaacatgcagcagcaccagccgctcaccatgcagcagccccttgggaaccagctccccatgcaggtccagtctgccttacactcacccaccatgcagcaaggatttactcttcaacccgactatcagactattatcaatcctacatctacagctgcacaagttgtcacccaggcaatggagtatgtgcgttcggggtgcagaaatcctcccccacaaccggtggactggaataacgactactgcagtagtgggggcatgcagagggacaaagcactgtaccttacttgagaatctgaacacctcttctttccactgaggaattcagggaagtgttttcaccatggattgctttgtacagtcaaggcagttctccattttattagaaaatacaagttgctaagcacttaggaccatttgagcttgtgggtcacccactctggaagaaatagtcatgcttctttattatttttttaatcctttatggacattgtttttcttcttccctgaaggaaatttggaccattcagattttatgttggttttttgctgtgaagtgctgcgctctagtaactgccttagcaactgtagatgtctcggataaaagtcctggattttccattggttttcataatgggtgtttatatgaaactactaaagactttttaaatggcttgatgtagcagtcatagcaagtttgtaaatagcatctatgttacactctcctagagtataaaatgtgaatgtttttgtagctaaattgtaattgaaactggctcattccagtttattgatttcacaataggggttaaattggcaaacattcatatttttacttcatttttaaaacaactgactgatagttctatattttcaaaatatttgaaaataaaaagtattcccaagtgattttaatttaaaaacaaattggctttgtctcattgatcagacaaaaagaaactagtattaagggaagcgcaaacacatttattttgtactgcagaaaaattgcttttttgtatcactttttgtgtaatggttagtaaatgtcatttaagtccttttatgtataaaactgccaaatgcttacctggtattttattagatgcagaaacagattggaaacagctaaattacaacttttacatatggctctgtcttattgtttcttcatactgtgtctgtatttaatctttttttatggaacctgttgcgcctatttatgaaataataaatataggtgtttgtaagtaaatttgttagtatttgaaagaggtttctttgatgttttaacttttgctggcaaaaaaaaattcacgcttggtgtgaatactttattatttagtttttacagtaacatgaataaagccaaacctgcttttcatttagcagcaaattaaagtaaccagtccttatttctgcatttctttggttgatgcaaacaaaaaactattatatttaagaactttatttcttcatacgacataacagaattgccctccaagtcacacaagctccaagactaaacaaacagacaggtcctctgtcttaaaaaggttacttcttggttctcagctggttctagtcaattctgaaccaccaccccccgccccccgcaaaaaagtaaaagtcaaaccaaacttcctcaagctgcatgcttttcacaaaatccagaaagcatttaagaattgaactaggggctggaagaagtgaaagggaagcatctaaaaatgaaaggtgagtaaccagatagcaaaagaaaagggaaagccatccaaatttgaaagctgttgatagaaattgagattcttgctgtcttttgtgcctctacaagctactactcattccagaattcctgggtcttccaagaggattcttaaggtaccagagatttgctagggaaccaaaagtgcttgagaatctgcctgagggcttgcatagctttcacattaaaaaaagaaaaagctagcagatttactcctttttaggggatcatatcaagaaagttagtctggttggaaaccaagagaatggctgatgtctctttcttggaatatgtgaaataaatttagcagtttaactaaatacaaatatatgcattgtgtaatccactcagaattaaacagacaaaaggtatgcttgctttggaatgattttaggcattgtacaaccttgaatcacttgagcatgtaataactaataaataatgcagatccatgtgattattaaaatgactgtagctgagagctctaattttcctgtcttgaaactgtataagaactcatgtgattaagttcacagtttattgtttgtctgtttagtattttagaaatataccagcactactaattaactaatgtcttttatttattatattatgataaagtaaaaatttcacttgcattaagtctaaactgagaaggtaattactgggaggagaatgagcagctttgactttgacaggcggtttgtgcaggaaagcacagtgccgtgttgtttacagcttttctagagcagctgtgcgaccagggtagagagtgttgaaattcaataccaaatacagtaaaaacaaatgtaaataaaagaaaacacatcatcaataaaactgttattatgcgtgaccgta

SEQ ID NO: 318 - RNA, 2719 nt, Homo sapiens
  source 1..2719: mol_type = genomic RNA
agagcgtcgccgagtcggagccggagcctgagccgcgcgctgtgtctccgctgcgtccgccgaggcccccgagtgtcagggacaaaagcctccgcctgctcccgcagacggggctcatctgccgccgccgccgcgctgaggagagttcgccgccgtcgccgcccgtgaggatctgagagccatgtcggccagcagcctcttggagcagtacaaaatggatctgtacatcaaaaggatggattaaacgatgatgattttgaaccttacttgagtccacaggcaaggcccaataatgcatatactgccatgtcagattcctacttacccagttactacagtccctccattggcttctcctattctttgggtgaagctgcttggtctacggggggtgacacagccatgccctacttaacttcttatggacagctgagcaacggagagccccacttcctaccagatgcaatgtttgggcaaccaggagccctaggtagcactccatttcttggtcagcatggttttaatttctttcccagtgggattgacttctcagcatggggaaataacagttctcagggacagtctactcagagctctggatatagtagcaattatgcttatgcacctagctccttaggtggagccatgattgatggacagtcagcttttgccaatgagaccctcaataaggctcctggcatgaatactatagaccaagggatggcagcactgaagttgggtagcacagaagttgcaagcaatgttccaaaagttgtaggttctgctgttggtagcgggtccattactagtaacatcgtggcttccaatagtttgcctccagccaccattgctcctccaaaaccagcatcttgggctgatattgctagcaagcctgcaaaacagcaacctaaactgaagaccaagaatggcattgcagggtcaagtcttccgccacccccgataaagcataacatggatattggaacttgggataacaagggtcccgttgcaaaagccccctcacaggctttggttcagaatataggtcagccaacccaggggtctcctcagcctgtaggtcagcaggctaacaatagcccaccagtggctcaggcatcagtagggcaacagacacagccattgcctccacctccaccacagcctgcccagctttcagtccagcaacaggcagctcagccaacccgctgggtagcacctcggaaccgtggcagtgggttcggtcataatggggtggatggtaatggagtaggacagtctcaggctggttctggatctactccttcagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaataaaaggcagttctacacagactgcagcaacggttgcatctgcatatcctaagaggaaaaaatgaccttcaagagaattaggacttttttcttaatttcactgacttcagagacgattgcagacttgcagtttaagtattggaatttcacaaaagacataggacttaactggaaaatgaaaaaaaaaagaaaaagaaaaaactaaacaaaaaatccctctaggtagtttaggtgaaaaatgtcccttttattttggctttggttgtgatttcagagcataatgctatgtttttttgtctttttactatgtttttcggatttttaagtccgtaagtgcatacagttttctctaatttttaaaccctttcctcctcccattttgacatttgcacttggagaacacttgagttgtgaaggttttgggcatccaccccagaaagtgggaatttgattttatccttccgaactggaagaacatttttatgaagaatttttgtctaggagaatataacagtgttacccaaggttgtgtctttaagggtggttcattttctctgaccttttgttactcaaagtaaagtactaggagtcctaagaaatgttctgttcttgtacattatactgattaagtcaggattaatttgatttcaaagctgagaacagtggtaaaaactcgtttacagaaatgcattttggaagagaaaaatactgtaaaacgtgtcgtgaatgtttcttcagtttcttgttcagccaatgaggaaagggcattgcctttctttttaccattaatcacttctcaataaacgtgagatcctgttgagcatca

SEQ ID NO: 319 - RNA, 2506 nt, Homo sapiens
  source 1..2506: mol_type = genomic RNA
agagtttctcaagaagccgagagagggacagaggctgagcctggggggagggctgcaggggactgagaggggagtgtaggcagtgggggtgagggggacagggaggaggtggagaagacggcaaggggcagcagggtgactggggcagagggagcccccaccccacgcccagggcaggccaggagtcataaggagaggggacaacaggaaggggcgacaaacggggaggggaaagggaggcggaggggcagcgaagtgaagcagggagggtcgcagtgtgaacaagagtgcgaggaagggtcaaggtggagggaggagaggggagagttagggagaaacagctgcaggagaggcgcggggaggaagccttaggggtgggggaagggagaggggagttgagaattagggaggaggtggtagagtccgggtagtgagcggagggacaggaagggtagggcaagaaagggagaggggacaggagggaagggtgggccaaagcggtgagaaaggagggccagccagttgggtgggggagagggccgaggcccgggggcaggagtgcagggctctgaggcggggagaggagaggagagaagagccgcggggggcccagcccggagccaggatgcccgcgccgcgcgcccgggagcagccccgcgtgcccggggagcgccagccgctgctgcctcgcggtgcgcggggccctcgacggtggcggcgggcggcgggcgcggccgtgctgctggtggagatgctggagcgcgccgccttcttcggcgtcaccgccaacctcgtgctgtacctcaacagcaccaacttcaactggaccggcgagcaggcgacgcgcgccgcgctggtattcctgggcgcctcctacctgctggcgcccgtgggcggctggctggccgacgtgtacctgggccgctaccgcgcggtcgcgctcagcctgctgctctacctggccgcctcgggcctgctgcccgccaccgccttccccgacggccgcagctccttctgcggagagatgcccgcgtcgccgctgggacctgcctgcccctcggccggctgcccgcgctcctcgcccagcccctactgcgcgcccgtcctctacgcgggcctgctgctactcggcctggccgccagctccgtccggagcaacctcacctccttcggtgccgaccaggtgatggatctcggccgcgacgccacccgccgcttcttcaactggttttactggagcatcaacctgggtgctgtgctgtcgctgctggtggtggcgtttattcagcagaacatcagcttcctgctgggctacagcatccctgtgggctgtgtgggcctggcatttttcatcttcctctttgccacccccgtcttcatcaccaagcccccgatgggcagccaagtgtcctctatgcttaagctcgctctccaaaactgctgcccccagctgtggcaacgacactcggccagagaccgtcaatgtgcccgcgtgctggccgacgagaggtctccccagccaggggcttccccgcaagaggacatcgccaacttccaggtgctggtgaagatcttgcccgtcatggtgaccctggtgccctactggatggtctacttccagatgcagtccacctatgtcctgcagggtcttcacctccacatcccaaacattttcccagccaacccggccaacatctctgtggccctgagagcccagggcagcagctacacgatcccggaagcctggctcctcctggccaatgttgtggtggtgctgattctggtccctctgaaggaccgcttgatcgaccctttactgctgcggtgcaagctgcttccctctgctctgcagaagatggcgctggggatgttctttggttttacctccgtcattgtggcaggagtcctggagatggagcgcttacactacatccaccacaacgagaccgtgtcccagcagattggggaggtcctgtacaacgcggcaccactgtccatctggtggcagatccctcagtacctgctcattgggatcagtgagatctttgccagcatcccaggcctggagtttgcctactcagaggccccgcgctccatgcagggcgccatcatgggcatcttcttctgcctgtcgggggtgggctcactgttgggctccagcctagtggcactgctgtccttgcccgggggctggctgcactgccccaaggactttgggaacatcaacaattgccggatggacctctacttcttcctgctggctggcattcaggccgtcacggctctcctatttgtctggatcgctggacgctatgagagggcgtcccagggcccagcctcccacagccgtttcagcagggacaggggctgaacaggccctattccagcccccttgcttcactctaccggacagacggcagcagtcccagctctggtttccttctcggtttattctgttagaatgaaatggttcccataaataaggggcatgagcccttcctcacga

SEQ ID NO: 320 - RNA, 3046 nt, Homo sapiens
  source 1..3046: mol_type = genomic RNA
ggagacgcggcggcgctggacgcggaggcgctgggcgcacggcgcggagccggccggagctcgaggccggcggcggcgggagagcgacccgggcggcctcgtagcggggccccggatccccgagtggcggccggagcctcgaaaagagattctcagcgctgattttgagatgatgggcttgggaaacgggcgtcgcagcatgaagtcgccgcccctcgtgctggccgccctggtggcctgcatcatcgtcttgggcttcaactactggattgcgagctcccggagcgtggacctccagacacggatcatggagctggaaggcagggtccgcagggcggctgcagagagaggcgccgtggagctgaagaagaacgagttccagggagagctggagaagcagcgggagcagcttgacaaaatccagtccagccacaacttccagctggagagcgtcaacaagctgtaccaggacgaaaaggcggttttggtgaataacatcaccacaggtgagaggctcatccgagtgctgcaagaccagttaaagaccctgcagaggaattacggcaggctgcagcaggatgtcctccagtttcagaagaaccagaccaacctggagaggaagttctcctacgacctgagccagtgcatcaatcagatgaaggaggtgaaggaacagtgtgaggagcgaatagaagaggtcaccaaaaaggggaatgaagctgtagcttccagagacctgagtgaaaacaacgaccagagacagcagctccaagccctcagtgagcctcagcccaggctgcaggcagcaggcctgccacacacagaggtgccacaagggaagggaaacgtgcttggtaacagcaagtcccagacaccagcccccagttccgaagtggttttggattcaaagagacaagttgagaaagaggaaaccaatgagatccaggtggtgaatgaggagcctcagagggacaggctgccgcaggagccaggccgggagcaggtggtggaagacagacctgtaggtggaagaggcttcgggggagccggagaactgggccagaccccacaggtgcaggctgccctgtcagtgagccaggaaaatccagagatggagggccctgagcgagaccagcttgtcatccccgacggacaggaggaggagcaggaagctgccggggaagggagaaaccagcagaaactgagaggagaagatgactacaacatggatgaaaatgaagcagaatctgagacagacaagcaagcagccctggcagggaatgacagaaacatagatgtttttaatgttgaagatcagaaaagagacaccataaatttacttgatcagcgtgaaaagcggaatcatacactctgaattgaactggaatcacatatttcacaacagggccgaagagatgactataaaatgttcatgagggactgaatactgaaaactgtgaaatgtactaaataaaatgtacatctgaagatgattattgtgaaattttagtatgcactttgtgtaggaaaaaatggaatggtcttttaaacagcttttggggggtactttggaagtgtctaataaggtgtcacaatttttggtagtaggtatttcgtgagaagttcaacaccaaaactggaacatagttctccttcaagtgttggcgacagcggggcttcctgattctggaatataactttgtgtaaattaacagccacctatagaagagtccatctgctgtgaaggagagacagagaactctgggttccgtcgtcctgtccacgtgctgtaccaagtgctggtgccagcctgttacctgttctcactgaaaagtctggctaatgctcttgtgtagtcacttctgattctgacaatcaatcaatcaatggcctagagcactgactgttaacacaaacgtcactagcaaagtagcaacagctttaagtctaaatacaaagctgttctgtgtgagaattttttaaaaggctacttgtataataacccttgtcatttttaatgtacaaaacgctattaagtggcttagaatttgaacatttgtggtctttatttactttgcttcgtgtgtgggcaaagcaacatcttccctaaatatatattaccaagaaaagcaagaagcagattaggtttttgacaaaacaaacaggccaaaagggggctgacctggagcagagcatggtgagaggcaaggcatgagagggcaagtttgttgtggacagatctgtgcctactttattactggagtaaaagaaaacaaagttcattgatgtcgaaggatatatacagtgttagaaattaggactgtttagaaaaacaggaatacaatggttgtttttatcatagtgtacacatttagcttgtggtaaatgactcacaaaactgattttaaaatcaagttaatgtgaattttgaaaattactacttaatcctaattcacaataacaatggcattaaggtttgacttgagttggttcttagtattatttatggtaaataggctcttaccacttgcaaataactggccacatcattaatgactgacttcccagtaaggctctctaaggggtaagtaggaggatccacaggatttgagatgctaaggccccagagatcgtttgatccaaccctcttattttcagaggggaaaatggggcctagaagttacagagcatctagctggtgcgctggcacccctggcctcacacagactcccgagtagctgggactacaggcacacagtcactgaagcaggccctgtttgcaattcacgttgccacctccaacttaaacattcttcatatgtgatgtccttagtcactaaggttaaactttcccacccagaaaaggcaacttagataaaatcttagagtactttcatactcttctaagtcctcttccagcctcactttgagtcctccttggggttgataggaattttctcttgctttctcaataaagtctctattcatctcatgtttaatttgtacgcatagaattgctgagaaataaaatgttctgttcaactta

SEQ ID NO: 321 - RNA, 3892 nt, Homo sapiens
  source 1..3892: mol_type = genomic RNA
acttcccctcaacgcgctgctcagggatgaccttcggcactgtgcttcttctgagtgtcctggcttcttatcatggattcaacctggatgtggaggagcctacgatcttccaggaggatgcaggcggctttgggcagagcgtggtgcagttcggtggatctcgactcgtggtgggagcacccctggaggtggtggcggccaaccagacgggacggctgtatgactgcgcagctgccaccggcatgtgccagcccatcccgctgcacatccgccctgaggccgtgaacatgtccttgggcctgaccctggcagcctccaccaacggctcccggctcctggcctgtggcccgaccctgcacagagtctgtggggagaactcatactcaaagggttcctgcctcctgctgggctcgcgctgggagatcatccagacagtccccgacgccacgccagagtgtccacatcaagagatggacatcgtcttcctgattgacggctctggaagcattgaccaaaatgactttaaccagatgaagggctttgtccaagctgtcatgggccagtttgagggcactgacaccctgtttgcactgatgcagtactcaaacctcctgaagatccacttcaccttcacccaattccggaccagcccgagccagcagagcctggtggatcccatcgtccaactgaaaggcctgacgttcacggccacgggcatcctgacagtggtgacacagctatttcatcataagaatggggcccgaaaaagtgccaagaagatcctcattgtcatcacagatgggcagaagtacaaagaccccctggaatacagtgatgtcatcccccaggcagagaaggctggcatcatccgctacgctatcggggtgggacacgctttccagggacccactgccaggcaggagctgaataccatcagctcagcgcctccgcaggaccacgtgttcaaggtggacaactttgcagcccttggcagcatccagaagcagctgcaggagaagatctatgcagttgagggaacccagtccagggcaagcagctccttccagcacgagatgtcccaagaaggcttcagcacagccctcacaatggatggcctcttcctgggggctgtggggagctttagctggtctggaggtgccttcctgtatcccccaaatatgagccccaccttcatcaacatgtctcaggagaatgtggacatgagggactcttacctgggttactccaccgagctagccctgtggaagggggtacagaacctggtcctgggggccccccgctaccagcataccgggaaggctgtcatcttcacccaggtgtccaggcaatggaggaagaaggccgaagtcacagggacgcagatcggctcctacttcggggcctccctctgctctgtggatgtggacagcgatggcagcaccgacctgatcctcattggggccccccattactatgagcagacccgagggggccaggtgtccgtgtgtcccttgcctagggggcagagggtgcagtggcagtgtgacgctgttctccgtggtgagcagggccacccctggggccgctttggggcagccctgacagtgttgggggatgtgaatgaggacaagctgatagacgtggccattggggccccgggagagcaggagaaccggggtgctgtctacctgtttcacggagcctcagaatccggcatcagcccctcccacagccagcggattgccagctcccagctctcccccaggctgcagtattttgggcaggcgctgagtgggggtcaggacctcacccaggatggactgatggacctggccgtgggggcccggggccaggtgctcctgctcaggagtctgccggtgctgaaagtgggggtggccatgagattcagccctgtggaggtggccaaggctgtgtaccggtgctgggaagagaagcccagtgccctggaagctggggacgccaccgtctgtctcaccatccagaaaagctcactggaccagctaggtgacatccaaagctctgtcaggtttgatctggcactggacccaggtcgtctgacttctcgtgccattttcaatgaaaccaagaaccccactttgactcgaagaaaaaccctgggactggggattcactgtgaaaccctgaagctgcttttgccagattgtgtggaggatgtggtgagccccatcattctgcacctcaacttctcactggtgagagagcccatcccctccccccagaacctgcgtcctgtgctggccgtgggctcacaagacctcttcactgcttctctccccttcgagaagaactgtgggcaagatggcctctgtgaaggggacctgggtgtcaccctcagcttctcaggcctgcagaccctgaccgtggggagctccctggagctcaacgtgattgtgactgtgtggaacgcaggtgaggattcctacggaaccgtggtcagcctctactatccagcagggctgtcgcaccgacgggtgtcaggagcccagaagcagccccatcagagtgccctgcgcctggcatgtgagacagtgcccactgaggatgagggcctaagaagcagccgctgcagtgtcaaccaccccatcttccatgagggctctaacggcaccttcatagtcacattcgatgtctcctacaaggccaccctgggagacaggatgcttatgagggccagtgcaagcagtgagaacaataaggcttcaagcagcaaggccaccttccagctggagctcccggtgaagtatgcagtctacaccatgatcagcaggcaggaagaatccaccaagtacttcaactttgcaacctccgatgagaagaaaatgaaagaggctgagcatcgataccgtgtgaataacctcagccagcgagatctggccatcagcattaacttctgggttcctgtcctgctgaacggggtggctgtgtgggatgtggtcatggaggccccatctcagagtctcccctgtgtttcagagagaaaacctccccagcattctgacttcctgacccagatttcaagaagtcccatgctggactgctccattgctgactgcctgcagttccgctgtgacgtcccctccttcagcgtccaggaggagctggatttcaccctgaagggcaatctcagtttcggctgggtccgcgagacattgcagaagaaggtgttggtcgtgagtgtggctgaaattacgttcgacacatccgtgtactcccagcttccaggacaggaggcatttatgagagctcagatggagatggtgctagaagaagacgaggtctacaatgccattcccatcatcatgggcagctctgtgggggctctgctactgctggcgctcatcacagccacactgtacaagcttggcttcttcaaacgccactacaaggaaatgctggaggacaagcctgaagacactgccacattcagtggggacgatttcagctgtgtggccccaaatgtgcctttgtcctaataatccactttcctgtttatctctaccactgtgggctggacttgcttgcaaccataaatcaacttacatggaaacaacttctgcatagatctgcactggcctaagcaacctaccaggtgctaagcaccttctcggagagatagagattgtaatgtttttacatatctgtccatctttttcagcaatgacccactttttacagaagcaggcatggtgccagcataaattttcatatgcttaagaattgtcacatgaaatgaggatgtttatagcacactttccttgcgtggaagagctataacccagggacctgagtgcctctctgggaatagtcgggggaacctatttgtgggcattgaaaaagttttttcactttc

SEQ ID NO: 322 - RNA, 3687 nt, Homo sapiens
  source 1..3687: mol_type = genomic RNA
agtcgagggctagcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgtttta

SEQ ID NO: 323 - RNA, 4897 nt, Homo sapiens
  source 1..4897: mol_type = genomic RNA
agagagagctgtggttgccggaagttgagcggcggcaagaaataatggcggcagctacgggggatcctggactctctaaactgcagtttgccccttttagtagtgccttggatgttgggttttggcatgagttgacccagaagaagctgaacgagtatcggctggatgaagctcccaaggacattaagggttattactacaatggtgactctgctgggctgccagctcgcttaacattggagttcagtgcttttgacatgagtgctcccaccccagcccgttgctgcccagctattggaacactgtataacaccaacacactcgagtctttcaagactgcagataagaagctccttttggaacaagcagcaaatgagatatgggaatccataaaatcaggcactgctcttgaaaaccctgtactcctcaacaagttcctcctcttgacatttgcagatctaaagaagtaccacttctactattggttttgctatcctgccctctgtcttccagagagtttacctctcattcaggggccagtgggtttggatcaaaggttttcactaaaacagattgaagcactagagtgtgcatatgataatctttgtcaaacagaaggagtcacagctcttccttacttcttaatcaagtatgatgagaacatggtgctggtttccttgcttaaacactacagtgatttcttccaaggtcaaaggacgaagataacaattggtgtatatgatccctgtaacttagcccagtaccctggatggcctttgaggaattttttggtcctagcagcccacagatggagtagcagtttccagtctgttgaagttgtttgcttccgtgaccgtaccatgcagggggcgagagacgttgcccacagcatcatcttcgaagtgaagcttccagaaatggcatttagcccagattgtcctaaagcagttggatgggaaaagaaccagaaaggaggcatgggaccaaggatggtgaacctcagtgaatgtatggaccctaaaaggttagctgagtcatcagtggatctaaatctcaaactgatgtgttggagattggttcctactttagacttggacaaggttgtgtctgtcaaatgtctgctgcttggagccggcaccttgggttgcaatgtagctaggacgttgatgggttggggcgtgagacacatcacatttgtggacaatgccaagatctcctactccaatcctgtgaggcagcctctctatgagtttgaagattgcctagggggtggtaagcccaaggctctggcagcagcggaccggctccagaaaatattccccggtgtgaatgccagaggattcaacatgagcatacctatgcctgggcatccagtgaacttctccagtgtcactctggagcaagcccgcagagatgtggagcaactggagcagctcatcgaaagccatgatgtcgtcttcctattgatggacaccagggagagccggtggcttcctgccgtcattgctgcaagcaagagaaagctggtcatcaatgctgctttgggatttgacacatttgttgtcatgagacatggtctgaagaaaccaaagcagcaaggagctggggacttgtgtccaaaccaccctgtggcatctgctgacctcctgggctcatcgctttttgccaacatccctggttacaagcttggctgctacttctgcaatgatgtggtggccccaggagattcaaccagagaccggaccttggaccagcagtgcactgtgagtcgtccaggactggccgtgattgcaggagccctggccgtggaattgatggtatctgttttgcagcatccagaagggggctatgccattgccagcagcagtgacgatcggatgaatgagcctccaacctctcttgggcttgtgcctcaccaggttcttgatcaatatgaacgagaaggatttaacttcctagccaaggtgtttaattcttcacattccttcttagaagacttgactggtcttacattgctgcatcaagaaacccaagctgctgagatctgggacatgagcgatgatgagaccatctgagatggccccgctgtggggctgacttctccccggccgcctgctgaggagctctccatcgccagagcaggactgctgaccccaggcctggtgattctgggcccctcctccataccccgaggtctgggattcccccctctgctgcccaggagtggccagtgttcggcgttgctcgggattcaagataccaccagttcagagctaaataataaccttggccttggccttgctattgacctgggacttggtcctccatgcagtttttatttcttgtcacagtgactgatagccatcccccaggatcctttccccttggccctgagggggtgacccaacacagaccaaatggggaaatgagcaaccagctcctgcccagagccactgcgggaggtggcaccctcatccccggaatgtgctgcccaccgcaccgcaggctcctcctgtgggggccctgggcatgggtgagggtgggaccccgtgagcgcactgcaccctggccctggtggagcgggaggaggaggagagccgagctgggtacgagactaaagggcccacatgacccagtgacgccagatttccaccaaggactgagtgagctgctcagacatggctttctgcctcccagcctgtcctccactgtgggcatagcatctgtgcctgcctgcctgcttgagggagaggagtttctgctgctgccttgagctggggggaagagcccaggggcagatcctggcagctgcctggatggggctcctccctgcccttatgagcaggccaggcccagaaaggccgagcctgggctgccttcctgccccagccgagggaggggtcagacggctctaccatgggtaactcaggcaagagctggttttcctctttattctgggtgtgtgcagctgtgaggccccaacccaggagaggccatggcctaggtacctgtgaccaccctgcccccgtgtagagggcatcgtctttcctgctattttattctttcagcttttgtcttaggcccagaatcaaagtgaaaattgagtcgagctgacccttacaacagtaggatttagtagggtagatttcaaatgaggcttcgcttctcccaaagtagccagtccaagttccagtggctgtcgttcagctcatgggagcttcatggggacacagccggcacaggtgcagggcccgagtccgcccacccagcctggcgctgaaactgcacacgtacactatgtggtttaagagcactttattattgttcttaaggctacttttaagtacaaaaaaagatggcctgccaaacctttttttttcttcttccaggaaaaacaggccacagagaatggtatattacagatttacacacatgaagagaaggtcagagcgcactgcaggcagcgcggctctgggaagaacttcacggagccccttcttagagcagggagggggctttctcagtgaaatgtttggttttctgctgcctcctctgccccaggcccccctccagggtactgcctatcccagataggtcagtgcaccagggacccggccgccagcaccgccgacccctcccagagtgacgcccttgttcactgacaaagagacctgtcccaggagtgtcctccaccgagccggtcagctgtgggtggttttcctgttacgacgctcagtagcctgtagcaataacaaactcgtggctatgaatgcagatgcagtgttctcatagaataactgttcctgcacttttacagacaaatctacgacaaaaaaaaagatcaactttttttttccgaacaacaaaaaaaatgaatgattacaataggaaagggaaaaattaaatagctacatatcattaacaaattaatgttcttcaaaaaatacctacaaatttctctgtacattctttacgcacagcgtaacgatggtctcaaaatcacccatatagaaaagtgttctcaacgatttttcctacagaaaatataggggcctgaatgccaaagcttggaagcccagtacagtgggagtgaaatgtgtgcggggcaaggagaagggcttttctttcctccacttttcaaaggcctgcagccactctgtgactacaagagccagtcctccgaccttttcacccagtgccaatttccaaaattcaacagctaaaaactgtaaaaccgggggtcatacggtgtgcagagtccacaaagccttgcaggtgaggtgaccacgcccacgtcacctggtcaggtgccatcgtcgtgagcctctggtgggccaggtgggacacagcacaccccagggggaggggatagaaacgctcattgaccaaaaaggagcagctgtgacctccacagctgtgtctgtcatgcttgcttcatctaatttctagttagtagctattaatatagcaaataataaatgcagtaataacagtataaagtcagaggaatgtatactgccttggccccagcgtacgaggaagcgtataaaacaccatatcacagattgtctgtcagtaatctgctgttcagccaagagagttcaaagggagcagtttctgcatgtagggaagttggaagacacaaaccccacctcccctgggagcttgtaacaaagcagacagggatgcaaaaataaatgatgtcagcctgcagccaaactccagcatcccacaccgcagctgacccactgctcatcgcgagggcctgccaggagctggcctcccgcactacttgtgagtaaagtgaatatcaaataccaatcttagagtacaactgtaccagcagtaagtatatctaggactgtaactgacaaaaataaactaattctgaaaagaa

SEQ ID NO: 324 - RNA, 3389 nt, Homo sapiens
  source 1..3389: mol_type = genomic RNA
acagctgcaccgccgagctgcgagcggctgcgagcgagagagcgtaagagcaagagagctagagagcgagcaacgggcactcgccccacgcctcccctcagccccaccgcgcgctccgcttgcctctccaccccgcccgactctacccggcccggtccctgcgcgggcacagcccagagctctggggcggtgcaggcagcctcgggactctccggcgcgccgccgcgtccccagacaaaggcttggccggcggccccggcccgctgcgccctcgctccccgcctccccagctcttctccgctcttcccccccgcgcttggctcggcgcgctccggccggccgcaaagtttcccgggcggcagcggcggctgcgcctcgcttcagcgatggccgcggagctgagcatggggccagagctgcccaccagcccgctggccatggagtatgtcaacgacttcgacctgctcaagttcgacgtgaagaaggagccactggggcgcgcggagcgtccgggcaggccctgcacacgcctgcagccagccggctcggtgtcctccacaccgctcagcactccgtgtagctccgtgccctcgtcgcccagcttcagcccgaccgaacagaagacacacctcgaggatctgtactggatggcgagcaactaccagcagatgaaccccgaggcgctcaacctgacgcccgaggacgcggtggaagcgctcatcggctcgcacccagtgccacagccgctgcaaagcttcgacagctttcgcggcgctcaccaccaccaccatcaccaccaccctcacccgcaccacgcgtacccgggcgccggcgtggcccacgacgagctgggcccgcacgctcacccgcaccatcaccatcatcaccaagcgtcgccgccgccgtccagcgccgctagcccggcgcaacagctgcccactagccaccccgggcccgggccgcacgcgacggcctcggcgacggcggcgggcggcaacggcagcgtggaggaccgcttctccgacgaccagctcgtgtccatgtccgtgcgcgagctgaaccgccacctgcggggcttcaccaaggacgaggtgatccgcctgaagcagaagcggcggaccctgaagaaccggggctacgcccagtcttgcaggtataaacgcgtccagcagaagcaccacctggagaatgagaagacgcagctcattcagcaggtggagcagcttaagcaggaggtgtcccggctggcccgcgagagagacgcctacaaggtcaagtgcgagaaactcgccaactccggcttcagggaggcgggctccaccagcgacagcccctcctctcccgagttctttctgtgagtcgtggccggtcctggcccccgcccttgccccggcccggactccctgtcccacgtccctagtcccagactaccccggaccctgtccctgccgcggccccagccttgacctgtttgacttgagcgagagggaggaagggcgcgcgggccgcgggcgacgggcgggtgcgcgggcgggcaggggaccttggctaaggcgagagtagcgcacgccagcgccgcctcctagactcgagcagagccggagagagagacgagagggtgggaggtcccggagtaacttctctccaggctgaagggcggcgaggcatagtcccgagaagtcaccaaggccatctggagactcctggctttctgaactttgcgcgttaagccgggacagctgctttgctgcccggagagtagtccgcgccaggaagagagcaacgaggaaaggagagggactctggcgtcccggcaggcgagaggcgaggctgagcgaaagaaggaaggacagacggacctgtctgtcagagttcggagaacactggctctcagccctgagacacaggcctcagttaggacgctcggcgcccaaatctcatcagttttattgcctgctcgattatatagaaaaatacaaaaaatctgcattaaaaatattaatcctgcatgctggacatgtatggtaataatttctattttgtaccattttcttgtttaactttagcatgttgttgatcatggatcatactccccttgtttctttgggtgagaagggatcgcagtttggaaactccggcggctgcgtgcggggtttcagtcccagctgtaggcttgtaaatacccgccccgccaaaccgcatagagaacgtggcagcaagctgagggtctttgtttgggtttattattacggtatttttgtttgtaagttaaaaagaaaaaaaaaaagaaaaagttccgggcattttgcatcagaaaacaactttgtcttggggcacacttggaagttgcatgttttctttccttcccttatccccattcggtcctctttttcctctctcgctttagttttcaaccttgttggtgctgagagagagaaccgagaggtcccagtacaagggcagggcagggcagggaagctgccaagctccgcaccccagaggagtgttctggactacagccttgtcttatggtcaaattgatacccttaataagaaaggaaaggaaaggaaaacagatcctcccctctgctttttattgtaaccagaatcaccctgaggtcccttctgaaccctctgggcctgcgctaattgtaggagccacagcgctcctagggtgagaggcttagccatccctgaccctggcagtgcactggtaagcagacactgcactgaaccaactgctatgctcagaatgtaccagaaacccaaacattggcaagtaattttgcaactttcaagtgcgttctttagaccaatgcattgcgtttctttccctgcttttgagatagtaggaagagttcttggtggtgtccccccccttcaattcttcagttgtatagtagttatagggaagatatgggtgtttttctttattattacttttttttttctgcaggtcagtaaaaggatttaagttgcactgacaaaaataccaaaataaaagtgtatttttaagttcccatttgaaattgctggcgctgctggccggatgcatttttgagtttgtattagttgataaattaacagtaataacaagattgtatgaaccgcatggtgcttgcagttttaaatattgtggatatttgtcctgcatcagaaacgagctttggtttttacagattcaactgtgttgaaatcaaacctgccgcaacagaaattgtttttatttcatgtaaaataagggatcaatttcaaaccctgcttatgatatgaaaatattaaaacctagtctattgtagttttattcagactggtttctgttttttggttattaaaatggtttcctattttgcttattaaaaccatggaaatggtgtttatttagagga

SEQ ID NO: 325 - RNA, 6900 nt, Homo sapiens
  source 1..6900: mol_type = genomic RNA
cttgctggatcagaggctttaaaatcttttttcatcttctagctgtagctcgggctgcttgtcggcttggcctccccctcccccctttgctctctgcctcgtctttccccaggacttcgctattttgcttttttaaaaaaaggcaagaaagaactaaactcccccctccctctcctccagtcgggctgcacctctgccttgcactttgcacagaggtagagagcgcgcgagggagagagaggaaagaaaaaaaataataaagagagccaagcagaagaggaggcgagaagcatgaagtgttaactcccccgtgccaaggcccgcgccgcccggacagacgcccgccgcgcctccagccccgagcggacgccgcgcgcgccctgcctgcagcccgggccggcgaggcgagcccttccttatgcaaagcgcgcagcggagcggcgagcgggggacgccgcgcaccgggccgggctcctccagcttcgccgccgcagccaccaccgccgccaccgcagctcgcggaggatcttcccgagcctgaagccgccggctcggcgcgcaaggaggcgagcgagcaaggaggggccggggcgagcgagggagcacattggcgtgagcaggggggagggagggcgggcgcggggggcgcgggcagggcgggggggtgtgtgtgtgagcgcgctcggaggtttcgggccagccaccgccgcgcaagctagaagcgccccagcccggcaagctggctcacccgctggccacccagcacagcccgctggcccctctcctgcagcccatctggcggagcggcggcggcggcggcggcggcggcaggagaatggcatcagaactggcaatgagcaactccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccgtgcagctcggtgcccccttcccccagcttctcggcgcccagcccgggctcgggcagcgagcagaaggcgcacctggaagactactactggatgaccggctacccgcagcagctgaaccccgaggcgctgggcttcagccccgaggacgcggtcgaggcgctcatcagcaacagccaccagctccagggcggcttcgatggctacgcgcgcggggcgcagcagctggccgcggcggccggggccggtgccggcgcctccttgggcggcagcggcgaggagatgggccccgccgccgccgtggtgtccgccgtgatcgccgcggccgccgcgcagagcggcgcgggcccgcactaccaccaccaccaccaccacgccgccggccaccaccaccacccgacggccggcgcgcccggcgccgcgggcagcgcggccgcctcggccggtggcgctgggggcgcgggcggcggtggcccggccagcgctgggggcggcggcggcggcggcggcggcggaggcggcgggggcgcggcgggggcggggggcgccctgcacccgcaccacgccgccggcggcctgcacttcgacgaccgcttctccgacgagcagctggtgaccatgtctgtgcgcgagctgaaccggcagctgcgcggggtcagcaaggaggaggtgatccggctgaagcagaagaggcggaccctgaaaaaccgcggctatgcccagtcctgccgcttcaagagggtgcagcagagacacgtcctggagtcggagaagaaccagctgctgcagcaagtcgaccacctcaagcaggagatctccaggctggtgcgcgagagggacgcgtacaaggagaaatacgagaagttggtgagcagcggcttccgagaaaacggctcgagcagcgacaacccgtcctctcccgagtttttcatgtgagtctgacacgcgattccagctagccaccctgataagtgctccgcgggggtccggctcgggtgtgggcttgctagttctagagccatgctcgccaccacctcaccacccccacccccaccgagtttggcccccttggccccctacacacacacaaacccgcacgcacacaccacacacacacacacacacacacacacaccccacaccctgctcgagtttgtggtggtggtggctgttttaaactggggagggaatgggtgtctggctcatggattgccaatctgaaattctccataacttgctagcttgttttttttttttttttacacccccccgccccacccccggacttgcacaatgttcaatgatctcagcagagttcttcatgtgaaacgttgatcacctttgaagcctgcatcattcacatattttttcttcttcttccccttcagttcatgaactggtgttcattttctgtgtgtgtgtgtgttttattttgtttggatttttttttttaattttacttttagagcttgctgtgttgcccaccttttttccaacctccaccctcactccttctcaacccatctcttccgagatgaaagaaaaaaaaaagcaaagtttttttttcttctcctgagttcttcatgtgagattgagcttgcaaaggaaaaaaaaatgtgaaatgttatagacttgcagcgtgccgagttccatcgggttttttttttagcattgttatgctaaaatagagaaaaaaatcctcatgaaccttccacaatcaagcctgcatcaaccttctgggtgtgacttgtgagttttggccttgtgatgccaaatctgagagtttagtctgccattaaaaaaactcattctcatctcatgcattattatgcttgctactttgtcttagcaacaatgaactataactgtttcaaagactttatggaaaagagacattatattaataaaaaaaaaaagcctgcatgctggacatgtatggtataattattttttcctttttttttccttttggcttggaaatggacgttcgaagacttatagcatggcattcatacttttgttttattgcctcatgacttttttgagtttagaacaaaacagtgcaaccgtagagccttcttcccatgaaattttgcatctgctccaaaactgctttgagttactcagaacttcaacctcccaatgcactgaaggcattccttgtcaaagataccagaatgggttacacatttaacctggcaaacattgaagaactcttaatgttttctttttaataagaatgacgccccactttggggactaaaattgtgctattgccgagaagcagtctaaaatttattttttaaaaagagaaactgccccattatttttggtttgttttatttttattttatattttttggcttttggtcattgtcaaatgtggaatgctctgggtttctagtatataatttaattctagtttttataatctgttagcccagttaaaatgtatgctacagataaaggaatgttatagataaatttgaaagagttaggtctgtttagctgtagattttttaaacgattgatgcactaaattgtttactattgtgatgttaaggggggtagagtttgcaaggggactgtttaaaaaaagtagcttatacagcatgtgcttgcaacttaaatataagttgggtatgtgtagtctttgctataccactgactgtattgaaaaccaaagtattaagaggggaaacgcccctgtttatatctgtaggggtattttacattcaaaaatgtatgtttttttttcttttcaaaattaaagtatttgggactgaattgcactaagatataacctgcaagcatataatacaaaaaaaaattgcaaaactgtttagaacgctaataaaatttatgcagttataaaaatggcattactgcacagttttaagatgatgcagatttttttacagttgtattgtggtgcagaactggattttctgtaacttaaaaaaaaatccacagttttaaaggcaataatcagtaaatgttattttcagggactgacatcctgtctttaaaaagaaatgaaaagtaaatcttaccacaataaatataaaaaaatcttgtcagttacttttcttttacatattttgctgtgcaaaattgttttatatcttgagttactaactaaccacgcgtgttgttcctatgtgcttttctttcattttcaattctggttatatcaagaaaagaataatctacaataataaacggcatttttttttgattctgtactcagtttcttagtgtacagtttaactgggcccaacaacctcgttaaaagtgtaaaatgcatccttttctccagtggaaggattcctggaggaatagggagacagtaattcagggtgaaattataggctgttttttgaagtgaggaggctggccccatatactgattagcaatatttaatatagatgtaaattatgacctcatttttttctccccaaagttttcagttttcaaatgagttgagccataattgcccttggtaggaaaaacaaaacaaaacagtggaactaggcttcctgagcatggccctacacttctgatcaggagcaaagccatccatagacagaggagccggacaaatatggcgcatcagaggtggcttgcgcacatatgcattgaacggtaaagagaaacagcgcttgccttttcactaaagttgactatttttccttcttctcttacacaccgagattttcttgttagcaaggcctgacaagatttaacataaacatgacaaatcatagttgtttgttttgttttgcttttctctttaacactgaagatcatttgtcttaaataggaaaaagaaaatccactccttacttccatatttccaagtacatatctggtttaaactatgttatcaaatcatatttcaccgtgaatattcagtggagaacttctctacctggatgagctagtaatgatttcagatcatgctatccccagaaataaaagcaaaaaataatacctgtgtggaatataggctgtgctttgatttactggtatttaccccaaaataggctgtgtatgggggctgacttaaagatcccttggaaagactcaaaactaccttcactagtaggactcctaagcgctgacctatttttaaatgacacaaattcatgaaactaatgttacaaattcatgcagtttgcactcttagtcatcttcccctagcacaccaatagaatgttagacaaagccagcactgttttgaaaatacagccaaacacgatgacttttgttttgttttctgccgttcttaaaagaaaaaaagataatattgcaactctgactgaaagacttatttttaagaaaacaggttgtgtttggtgctgctaagttctggccagtttatcatctggccttcctgcctattttttacaaaacacgaagacagtgtgtaacctcgacattttgaccttcctttatgtgctagtttagacaggctcctgaatccacacttaattttgcttaacaaaagtcttaatagtaaacctcccctcatgagcttgaagtcaagtgttcttgacttcagatatttctttcctttttttttttttttcctcatcacaactaagagatacacaaactctgaagaagcagaaatggagagaatgcttttaacaaaaaagcatctgatgaaagattttaggcaaacattctcaaaataagagtgatattctggatgtagttattgcagttatctcatgacaaatgaggcctggattggaaggaaaatatagttgtgtagaattaagcattttgataggaatctacaaggtagttgaatataataagcaggtttgggcccccaaactttagaaaatcaaatgcaaaggtgctggcaaaaatgaggtttgagtggctggctgtaagagaaggttaactcctagtaaaaggcatttttagaaataacaattactgaaaactttgaagtatagtgggagtagcaaacaaatacatgttttttttttcttacaaagaactcctaaatcctgagtaagtgccattcattacaataagtctctaaatttaaaaaaaaaaaaatcatatgaggaaatctagctttcccctttacgctgcgtttgatctttgtctaaatagtgttaaaattcctttcattccaattacagaactgagcccactcgcaagttggagccatcagtgggatacgccacattttggaagccccagcatcgtgtacttaccagtgtgttcacaaaatgaaatttgtgtgagagctgtacattaaaaaaaatcatcattattattattatttgcagtcatggagaaccacctacccctgacttctgtttagtctcctttttaaataaaaattactgtgttagagaagaaggctattaaatgtagtagttaactatgcctcttgtctgggggtttcatagagaccggtaggaaagcgcactcctgcttttcgatttatggtgtgtgcaagtaaacaggtgcattgctttcaacctgccatactagttttaaaaattcactgaaattacaaagatacatatatatgcatatatataatggaaagtttcccggaatgcaacaattagcattttaaaatcatatataggcatgcacattctaaatagtactttttcatgcttcattgtttctctggcagataattttactaagaagaaaaatagatattcgactccccttccctaaacaaatccacgggcagaggctccagcggagccgagccccctggttttctcgtaggccctagacggtgttgcatttatcagtgatgtcaaacgtgctcatttgtcagacatagctgtaaatgaaaacaatgtgtggcaaaatacaaagttagttaaataca

SEQ ID NO: 326 - RNA, 2108 nt, Homo sapiens
  source 1..2108: mol_type = genomic RNA
ctttcaattcctctggcaacaaaccacacactgggatctgacactgtagagtgctttctcttctctttttttgggggggggagggggtgtggttgcatatttaaactctcacgcatttatgtactgaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 327 - RNA, 3292 nt, Homo sapiens
  source 1..3292: mol_type = genomic RNA
agttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 328 - RNA, 2097 nt, Homo sapiens
  source 1..2097: mol_type = genomic RNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 329 - RNA, 3301 nt, Homo sapiens
  source 1..3301: mol_type = genomic RNA
gcatttctcttctgtgctcactgccacacgcagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggcccccggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacacacagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctgcaggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggcagagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaagagaaaaaagaaatttagaagaataacaagttattccaaatgaaggcgtaagaaagggaataataacaataataagaggagttgttcatgaggaaaaaccaaagcttgaaaattcaacaaagccagtgaagctcattcttgaaaacatgaatcacactcatgaattctaactacaatgaaaaagagaaagaaagagcaggcatgcatttccatatgggagtgagccagcagacagccctacagatcgtacacacgttttccaaaactaacaatggaacaggcggcaaacctatgccaatatactagaaattgcagattaaatagatgaaatattctaaactggagtttacataatgaacataagagtaatcagagaatctgactcattttagatgtgtgtgtgtgtgtatatatatgtgtgtgtgtgtgaaaaacattgactataataaaaataatctcgagttca

SEQ ID NO: 330 - RNA, 966 nt, Homo sapiens
  source 1..966: mol_type = genomic RNA
gcagttcctgagagaagaaccctgaggaacagacgttccctcgcggccctggcacctccaaccccagatatgctgctgctgctgctgctgcccctgctctgggggagggagagggtggaaggacagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttcgtatggagaaaggaaatataaaatggaattataaatatgaccagctctctgtgaacgtgacagggtaacctgactgagtcctgggcagatgataacccccgacaccatggcctggctgcccactcctcaggggaggaaagagagatccagtatgcacccctcagctttcataagggggagcctcaggacctatcaggacaagaagccaccaacaatgagtactcagagatcaagatccccaagtaagaaaatgcagaggctcgggcttgtttgagggttcacgacccctccagcaaaggagtctgaggctgattccagtagaattagcagccctcaatgctgtgcaacaagacatcagaacttattcctcttgtctaactgaaaatgcatgcctgatgaccaaactctccctttccccatccaatcggtccacactccccgccctggcctctggtacccaccattctcctctgtacttctctaaggatgactactttagattccgaatatagtgagattgtaacgtg

SEQ ID NO: 331 - protein, 4 aa, unidentified
DEAD